(19) United States
(12) Patent Application Publication
(10) Pub. No.: US 2026/0250298 A1
(43) Pub. Date: Aug. 27, 2026

(54) High-Performance Organic Electroluminescent Device and Compound Thereof

(71) Applicants: CHENGDU BOE OPTOELECTRONICS TECHNOLOGY CO., LTD., Chengdu (CN); BOE TECHNOLOGY GROUP CO., LTD., Beijing (CN)

(72) Inventors: Xianwen MENG, BEIJING (CN); Rongrong GAO, BEIJING (CN); Juncong LI, BEIJING (CN); Wenzhu JING, BEIJING (CN); Lei CHEN, BEIJING (CN); Dongxu ZHANG, BEIJING (CN)

(73) Assignees: CHENGDU BOE OPTOELECTRONICS TECHNOLOGY CO., LTD., Chengdu (CN); BOE TECHNOLOGY GROUP CO., LTD., Beijing (CN)

(21) Appl. No.: 18/848,755

(22) PCT Filed: May 27, 2024
(86) PCT No.: PCT/CN2024/095468

§ 371 Date: Sep. 19, 2024

(30) Foreign Application Priority Data
Jul. 21, 2023 (WO) PCT/CN2023/108619

(51) Int. Cl. C07D 495/20; C07D 403/10; C07D 487/10; C07D 519/00; H10K 50/16

(52) U.S. Cl. CPC C07D 495/20; C07D 403/10; C07D 487/10; C07D 519/00; H10K 50/16

(57) ABSTRACT

The present disclosure relates to an azaspirocyclic group-containing organic compound represented by Formula O, electron transport material and organic electroluminescent device. This type of compound is used as an electron transport material in organic electroluminescent devices and exhibits good electroluminescent properties.

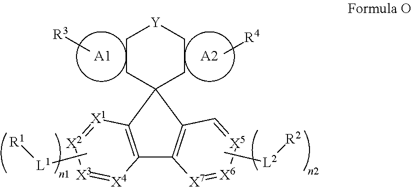

### CROSS-REFERENCE TO RELATED APPLICATIONS

The present application is a U.S. National Stage Application under 35 U.S.C. § 371 of International Patent Application No. PCT/CN2023/108619, filed on Jul. 21, 2023, the disclosure of which is incorporated by reference herein in its entirety.

### TECHNICAL FIELD

The present disclosure relates to the technical field of luminescent materials. Specifically, the present disclosure relates to an organic compound containing an azaspirocyclic group, an electron transport material, and an organic electroluminescent device.

### BACKGROUND ART

Organic light-emitting diode (OLED) refers to an organic electroluminescent material that has a phenomenon of emitting light under the action of electrical excitation. Compared with traditional LCD displays, as the latest display technology, OLED has the advantages of self-illumination, wide viewing angle, low power consumption, all-black display, full color, etc., and has broad commercial application potentials. In recent years, organic electroluminescent materials have also made impressive progress. After the commercialization of OLED flexible devices, relevant scientific research institutions and enterprises are carrying out work in this area. At present, research on organic electroluminescent materials has achieved great results, but there are still some problems that need to be solved in the commercialization process of OLED.

Under the driving action of the electric field, holes are injected from the anode and gradually transferred to the EML through the HOMO energy levels of functional layers such as HIL and HTL. At the same time, electrons are injected from the cathode and gradually transfer to the EML through the LUMO energy levels of functional layers such as EIL and ETL, and holes and electrons recombine in the EML to generate excitons. When an exciton returns from its excited state to its ground state, energy is emitted in the form of light. Therefore, how to balance the injection and recombination of holes and electrons is an important research content to make OLEDs have excellent luminous efficiency.

Organic electroluminescent devices are carrier injection devices, and the recombination efficiency of carriers affects the efficiency of the device. In order to achieve effective recombination of carriers in the light emitting layer and prevent excitons from diffusing to the surrounding functional layers, the functional layers adjacent to the light emitting layer are required to have a higher T1 energy level. Therefore, it is particularly important to develop electron transport materials with appropriate T1 energy levels to effectively improve device efficiency.

In OLED materials, since most organic electroluminescent materials transport holes faster than electrons, it is easy to cause an imbalance between numbers of electrons and holes in the light-emitting layer, so the efficiency of the device is relatively low. Therefore, it is very important to develop electron transport materials with high electron mobility.

In addition, because OLED devices generate Joule heat when operating under voltage, which makes organic materials prone to crystallization, affecting the life and efficiency of the devices. Therefore, it is also necessary to develop stable and efficient organic electroluminescent materials.

The electron transport material behaves as an electron-deficient system in its molecular structure and has strong electron-withdrawing groups, which makes the electron transport material have a certain polarity. Compared with the hole transport material, the electron transport material is easier to crystallize, which makes the electron transport material crystallize during the film formation process, resulting in poor film-forming ability of the material, and may even lead to hole blocking in the material, which is not conducive to the mass production output of OLED devices. Therefore, in the processes of developing and designing electron transport materials, the crystallization properties of the materials are also important performance indicators.

In short, electron transport materials with excellent transmission performance should have the following characteristics:
1. Appropriate HOMO and LUMO energy levels. The lower LUMO energy level is conducive to the injection of electrons from the cathode and reduces the turn-on voltage of the device. A lower HOMO energy level can more effectively confine hole carriers in the light-emitting layer, thereby improving the recombination efficiency of holes and electrons.
2. Higher triplet energy level. Since triplet excitons have a long lifetime and a large diffusion distance, the higher triplet energy level of the electron transport material can prevent the diffusion of triplet excitons from the light-emitting layer to the electron transport layer and improve the efficiency of the device.
3. Higher electron mobility. Mobility will affect the driving voltage of the device. Low electron mobility will increase the driving voltage of the device, thereby reducing the power efficiency of the device. In addition, if the driving voltage of the device is too high, it will also have a significant impact on the stability of the device.
4. It can form a good amorphous film to avoid performance degradation caused by crystallization.

The efficiency and lifespan of currently disclosed electron transport materials need to be improved, so developing stable and efficient electron transport materials to improve device efficiency and extend device life has important practical application value.

### Contents of the Invention

The inventors of the present disclosure have designed an organic small molecule material containing an azaspirocyclic group, and used it as an electron transport material in organic electroluminescent devices, which shows good electroluminescent performance.

Specifically, in the first aspect of the present disclosure, a compound represented by Formula O, or a stereoisomer or tautomer of the compound is provided,

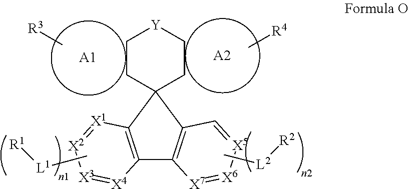

wherein:
X¹, X², X³, and X⁴ are each independently selected from CH and N, and at least one of X¹, X², X³ and X⁴ is N, and X³ and X⁴ are not N at the same time;
X⁵, X⁶, and X⁷ are each independently selected from CH and N, and X⁶ and X⁷ are not N at the same time;
Ring A1 and Ring A2 are each independently selected from C₆-C₁₂ aromatic rings;
Y is selected from —O—, —S—, —CR^{a}R^{b}—;
R^{a} and R^{b} are each independently selected from hydrogen and C₁-C₆ alkyl;
preferably, R^{a} and R^{b} are each independently selected from hydrogen and methyl;
preferably, Y is selected from —O—, —S—, —CH₂—,

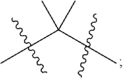 [SMILES: *C(*)(C)C]

more preferably, Y is selected from —O—, —S—;
n1 and n2 are each independently selected from 0 and 1, and n1 and n2 are not 0 at the same time;
L¹ and L² are each independently selected from the group consisting of a direct bond and C₆-C₁₈ arylene, and the C₆-C₁₈ arylene is optionally substituted by one R^{L};
R^{L} is selected from benzoxazolyl, benzothiazolyl, benzimidazolyl, benzisoxazolyl, benzisothiazolyl, and indazolyl;
R¹ and R² are each independently selected from the group consisting of diazaphenanthrolinyl, 6-membered nitrogen-containing heteroaryl, benzoxazolyl, benzothiazolyl, benzimidazolyl, benzisoxazolyl, benzisothiazolyl, indazolyl, and the diazaphenanthrolinyl, 6-membered nitrogen-containing heteroaryl, benzoxazolyl, benzothiazolyl, benzimidazolyl, benzisoxazolyl, benzisothiazolyl, indazolyl are each independently optionally substituted by one or two R^{R};
R^{R} is selected from C₆-C₁₈ aryl and fluorenyl, and the C₆-C₁₈ aryl and fluorenyl are each independently optionally substituted by 1 to 3 (preferably 1 to 2, more preferably 2) C₁-C₆ alkyl;
R³ and R⁴ are each independently selected from hydrogen and C₁-C₆ alkyl;
preferably, R³ and R⁴ are hydrogen.

In some embodiments, Ring A1 and Ring A2 are each independently selected from benzene ring and naphthalene ring.

In some embodiments, Ring A1 is selected from the group consisting of

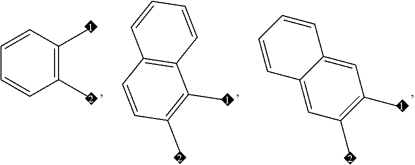

wherein Carbon atom No. 1 is fused with the carbon atom adjacent to Y on the middle 6-membered ring, and Carbon atom No. 2 is fused with the carbon atom far away from Y on the middle 6-membered ring.

In some embodiments, Ring A1 is

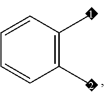

wherein Carbon atom No. 1 is fused with the carbon atom adjacent to Y on the middle 6-membered ring, and Carbon atom No. 2 is fused with the carbon atom far away from Y on the middle 6-membered ring.

In some embodiments, Ring A2 is selected from the group consisting of

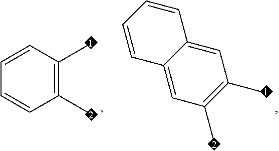

wherein Carbon atom No. 1 is fused with the carbon atom adjacent to Y on the middle 6-membered ring, and Carbon atom No. 2 is fused with the carbon atom far away from Y on the middle 6-membered ring.

In some embodiments, Ring A2 is

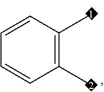

wherein Carbon atom No. 1 is fused with the carbon atom adjacent to Y on the middle 6-membered ring, and Carbon atom No. 2 is fused with the carbon atom far away from Y on the middle 6-membered ring.

In some embodiments,

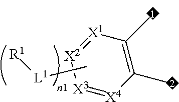

is selected from the group consisting of:

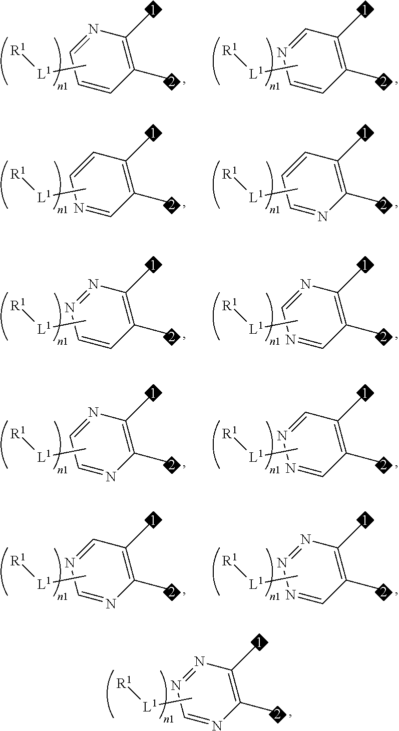

wherein, Carbon atom No. 1 is bonded to the central spiro carbon atom, and Carbon atom No. 2 is bonded to a carbon atom of the aromatic ring on the right.

In some embodiments,

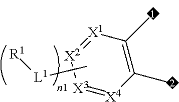

is selected from the group consisting of:

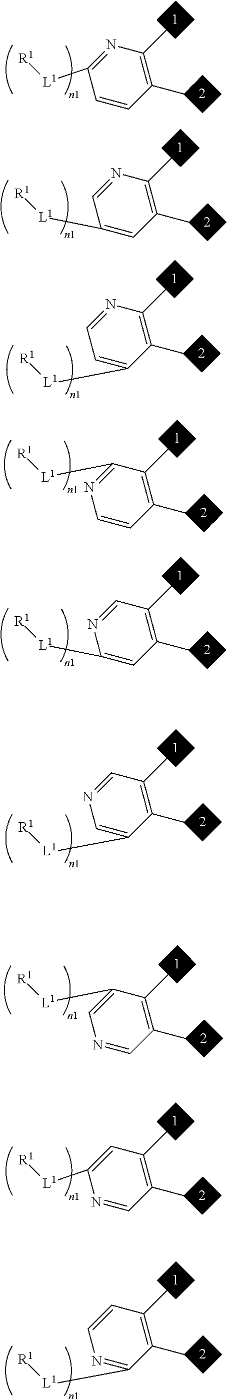

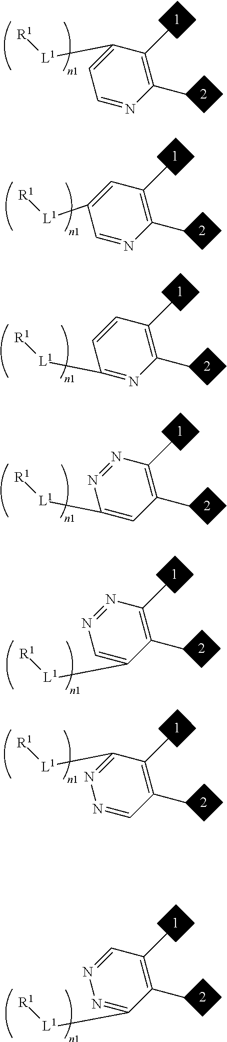

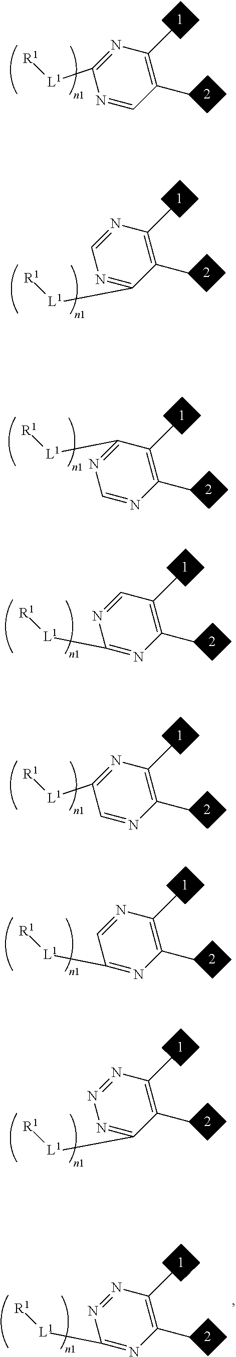

wherein, Carbon atom No. 1 is bonded to the central spiro carbon atom, and Carbon atom No. 2 is bonded to a carbon atom of the aromatic ring on the right.

In some embodiments,

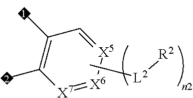

is selected from the group consisting of:

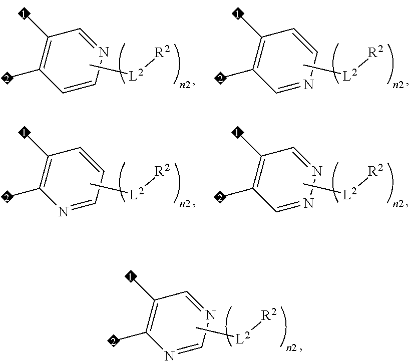

wherein, Carbon atom No. 1 is bonded to the central spiro carbon atom, and Carbon atom No. 2 is bonded to a carbon atom of the aromatic ring on the left.

In some embodiments,

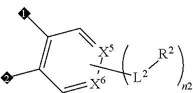

is selected from the group consisting of:

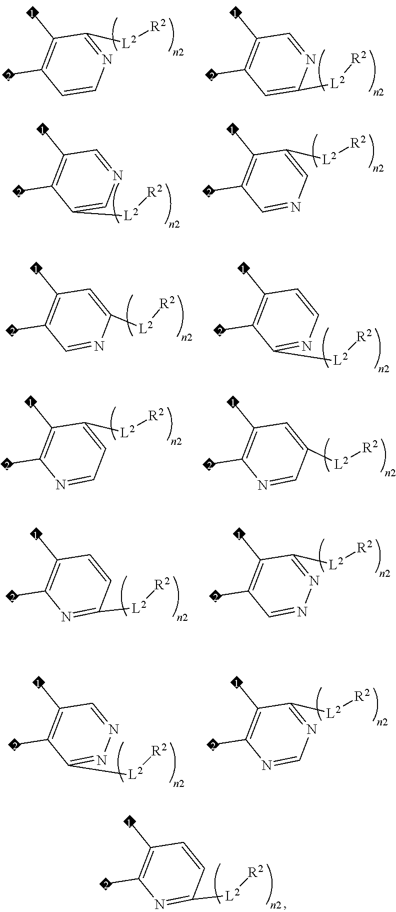

wherein, Carbon atom No. 1 is bonded to the central spiro carbon atom, and Carbon atom No. 2 is bonded to a carbon atom of the aromatic ring on the left.

In some embodiments, L¹ and L² are each independently selected from the group consisting of a direct bond, phenylene, naphthylene, and biphenylene, and the phenylene, naphthylene, and biphenylene are each independently optionally substituted by one R^{L}.

In some embodiments, L¹, L² are each independently selected from the group consisting of a direct bond, phenylene, naphthylene, biphenylene, and the phenylene is optionally substituted by one R^{L}.

In some embodiments, L¹ and L² are each independently selected from the group consisting of a direct bond,

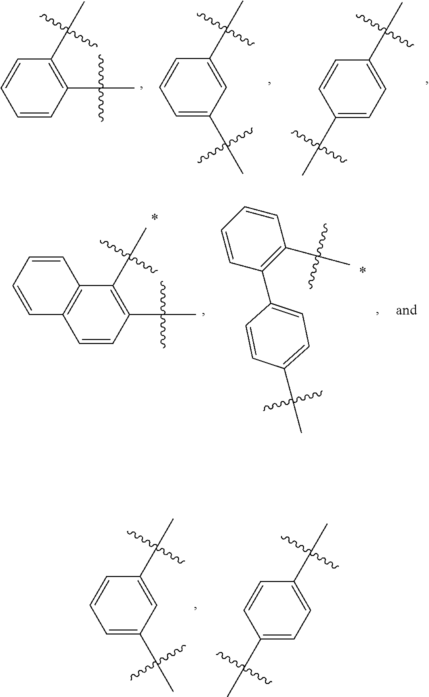

are each independently optionally substituted by one R^{L}, wherein the bond with marker * is bonded to the parent ring, and the bond without marker * is bonded to R¹ or R².

In some embodiments, R^{L} is selected from benzoxazolyl, benzothiazolyl.

In some embodiments, R^{L} is benzoxazolyl.

In some embodiments, R^{E} is selected from

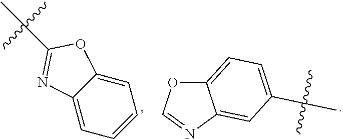

In some embodiments, L¹ and L² are each independently selected from the group consisting of:
a direct bond,

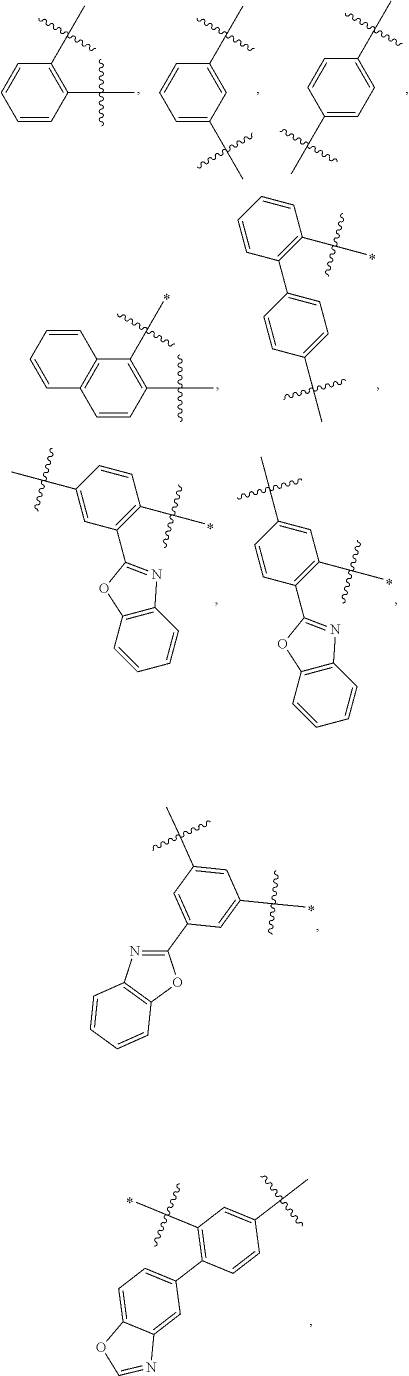

wherein, the bond with marker * is bonded to the parent ring, and the bond without marker * is bonded to R¹ or R².

In some embodiments, R¹ and R² are each independently selected from the group consisting of diazaphenanthrolinyl, pyridinyl, pyrimidinyl, triazinyl, benzoxazolyl, benzothiazolyl, and the diazaphenanthrolinyl, pyridinyl, pyrimidinyl, triazinyl, benzoxazolyl and benzothiazolyl are each independently optionally substituted by one or two R^{R}.

In some embodiments, R¹ and R² are each independently selected from the group consisting of diazaphenanthrolinyl, pyridinyl, pyrimidinyl, triazinyl, benzoxazolyl, benzothiazolyl, and the pyridinyl, pyrimidinyl, triazinyl and benzoxazolyl are each independently optionally substituted by one or two R^{R}.

In some embodiments, R¹ and R² are each independently selected from the group consisting of o-diazaphenanthrolinyl, pyridinyl, pyrimidinyl, 1,3,5-triazinyl, benzoxazolyl, and the pyridinyl, pyrimidinyl, 1,3,5-triazinyl, benzoxazolyl are each independently optionally substituted by one or two R^{R}.

In some embodiments, R¹ and R² are each independently selected from the group consisting of

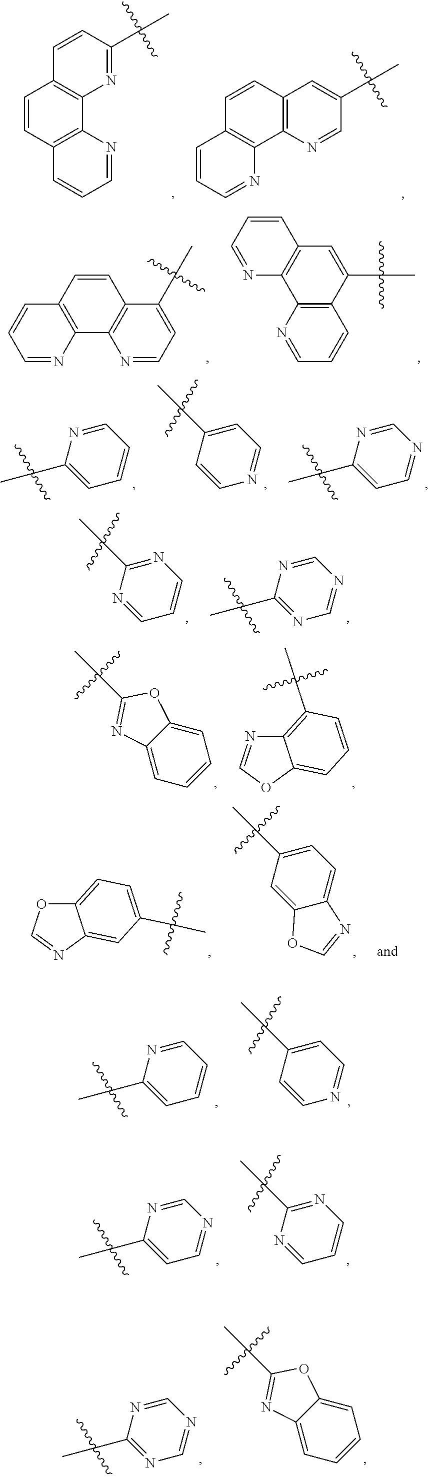

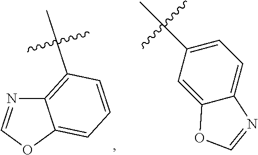 [SMILES: *c1cccc2ocnc12 ; *c1ccc2ncoc2c1]

are each independently optionally substituted by one or two R^{R}.

In some embodiments, R^{R} is selected from the group consisting of phenyl, biphenyl, naphthyl, and fluorenyl, and the phenyl, biphenyl, naphthyl, and fluorenyl each are independently optionally substituted by two C₁-C₆ alkyl groups.

In some embodiments, R^{R} is selected from the group consisting of phenyl, biphenyl, naphthyl, fluorenyl, and the fluorenyl is optionally substituted by two C₁-C₆ alkyl groups.

In some embodiments, R^{R} is selected from the group consisting of phenyl, biphenyl, naphthyl, fluorenyl, and the fluorenyl is optionally substituted by two methyl groups.

In some embodiments, R^{R} is selected from the group consisting of

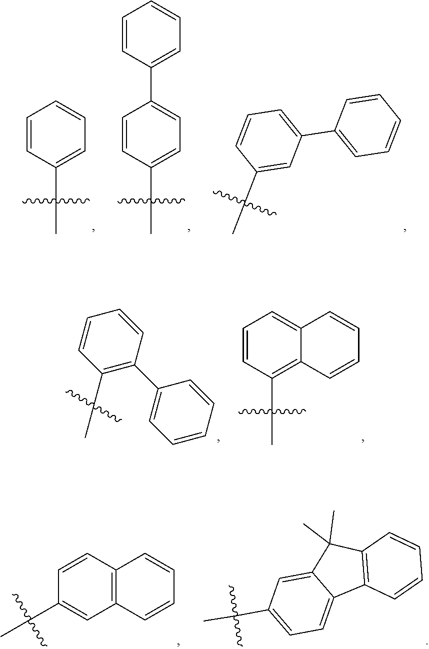

In some embodiments, R¹ and R² are each independently selected from the group consisting of:

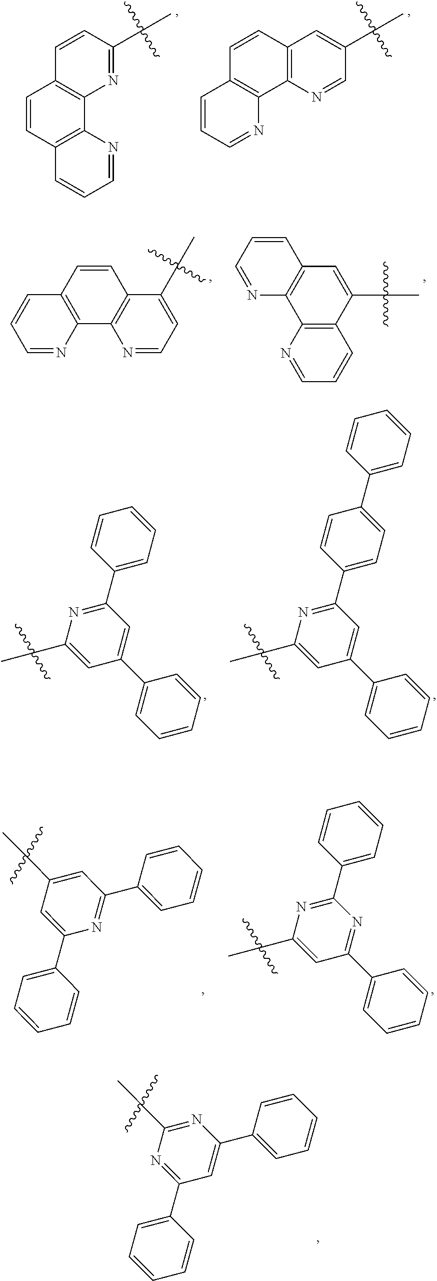

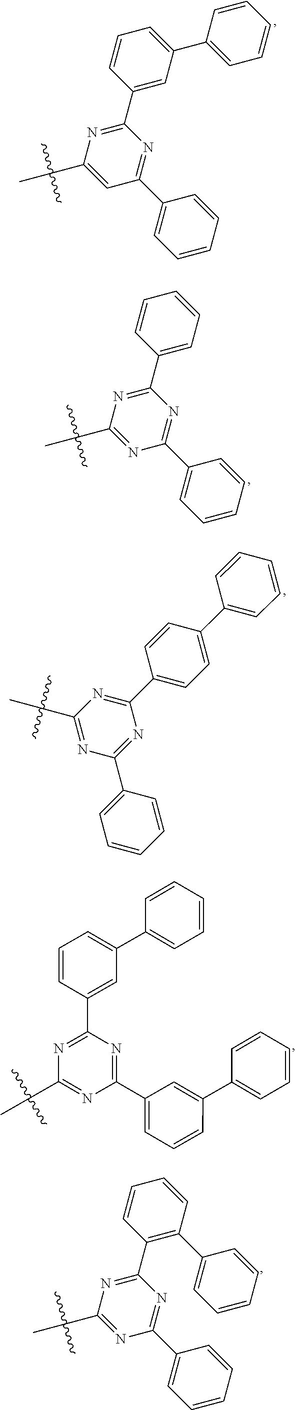

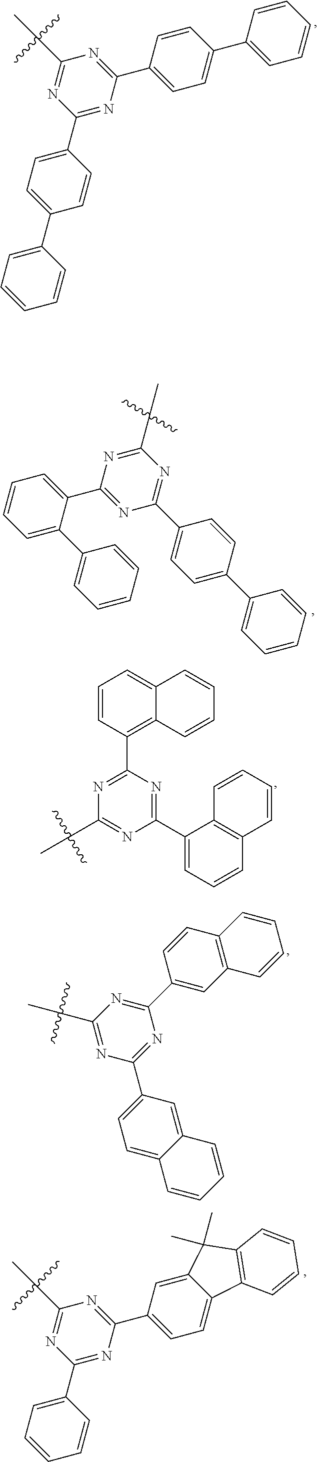

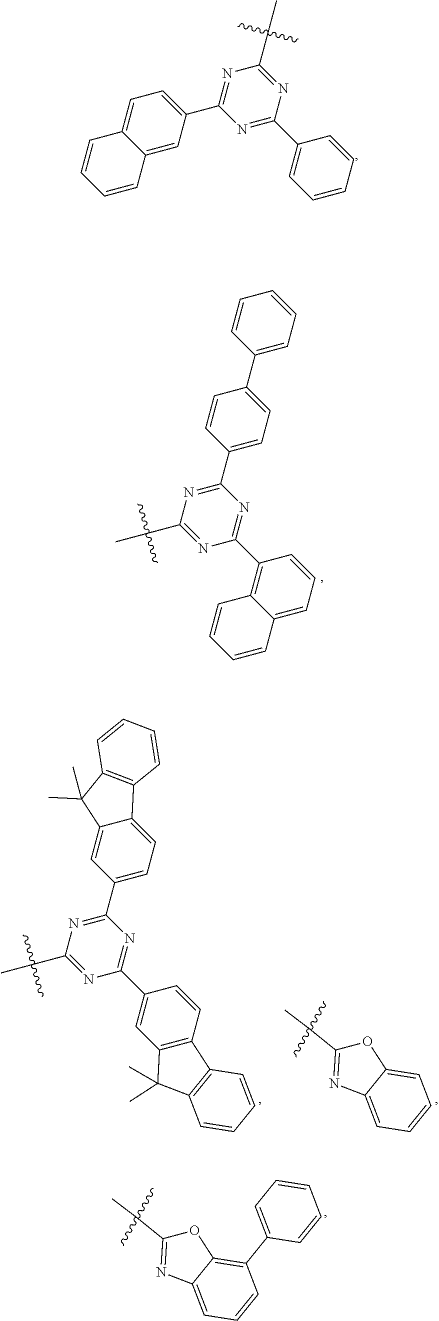 [SMILES: *c1nc(-c2ccccc2)nc(-c2ccc3ccccc3c2)n1 ; *c1nc(-c2ccc(-c3ccccc3)cc2)nc(-c2cccc3ccccc23)n1 ; *c1nc(-c2ccc3c(c2)C(C)(C)c2ccccc2-3)nc(-c2ccc3c(c2)C(C)(C)c2ccccc2-3)n1 ; *c1nc2ccccc2o1 ; *c1nc2cccc(-c3ccccc3)c2o1]

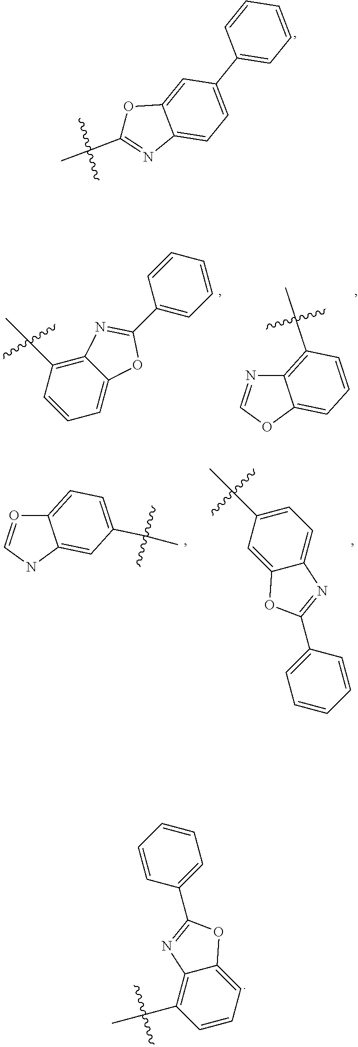

In some embodiments, R¹-L¹, R²-L²- are each independently selected from the group consisting of:

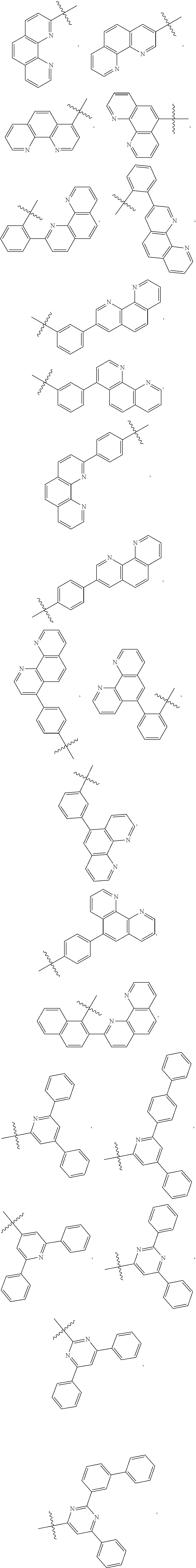

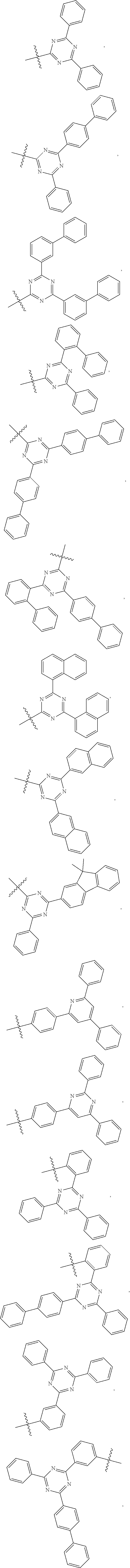

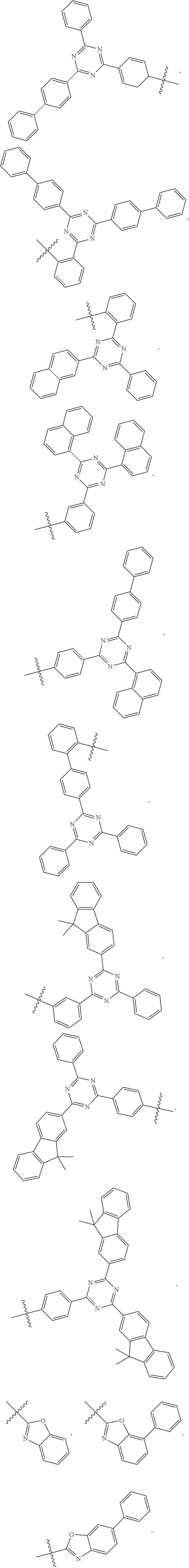

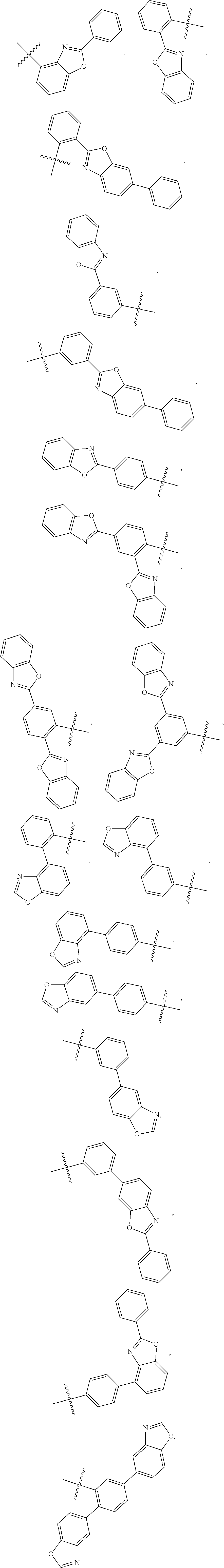

In the second aspect of the present disclosure, there is provided a compound represented by Formula I, or a stereoisomer or tautomer of the compound,

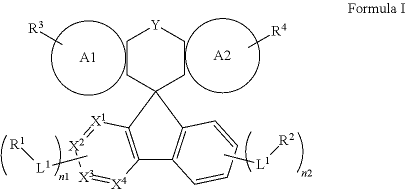

wherein:
X¹, X², X³, and X⁴ are each independently selected from CH and N, and at least one of X¹, X², X³, and X⁴ is N, and X³ and X⁴ are not N at the same time;
preferably, X¹, X², X³ and X⁴ are each independently selected from CH and N, and two of X¹, X², X³ and X⁴ are N, and X³ and X⁴ are not N at the same time;
n1 and n2 are each independently selected from 0 and 1, and n1 and n2 are not 0 at the same time;
preferably,

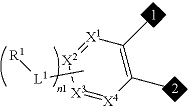

is selected from

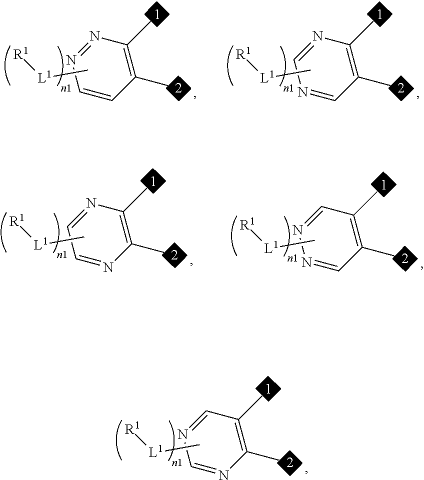

wherein Carbon atom No. 1 is bonded to the central spiro carbon atom, and Carbon atom No. 2 is bonded to a carbon atom of the aromatic ring on the right;
more preferably,

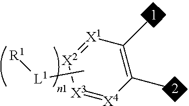

is selected from the group consisting of:

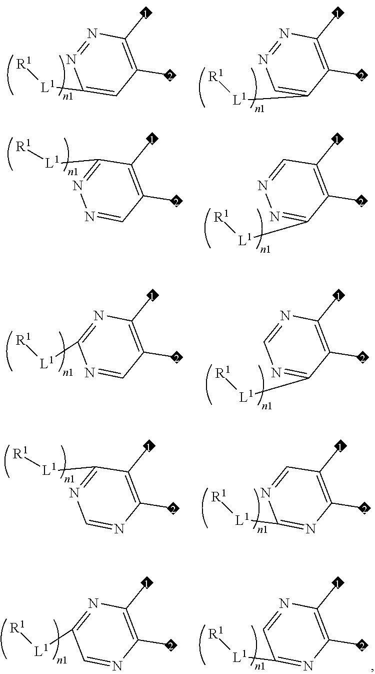

wherein, Carbon atom No. 1 is bonded to the central spiro carbon atom, and Carbon atom No. 2 is bonded to a carbon atom of the aromatic ring on the right;
preferably,

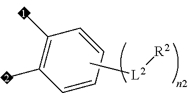

is selected from

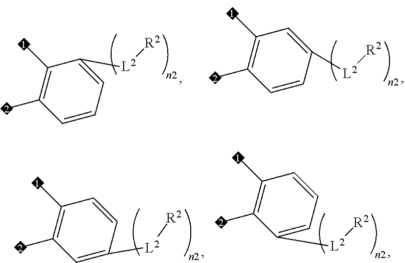

wherein Carbon atom No. 1 is bonded to the central spiro carbon atom, and Carbon atom No. 2 is bonded to a carbon atom of the aromatic ring on the left;
Ring A1 is selected from benzene ring and naphthalene ring;
preferably, Ring A1 is selected from the group consisting of

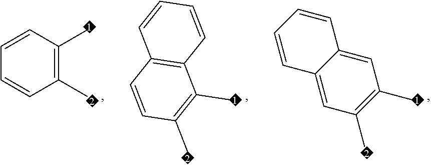

wherein Carbon atom No. 1 is fused with the carbon atom adjacent to Y on the middle 6-membered ring, and Carbon atom No. 2 is fused with the carbon atom far away from Y on the middle 6-membered ring;
more preferably, Ring A1 is

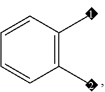

wherein Carbon atom No. 1 is fused with the carbon atom adjacent to Y on the middle 6-membered ring, and Carbon atom No. 2 is fused with the carbon atom far away from Y on the middle 6-membered ring;
Ring A2 is selected from benzene ring and naphthalene ring;
preferably, Ring A2 is selected from the group consisting of

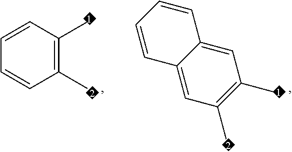

wherein Carbon atom No. 1 is fused with the carbon atom adjacent to Y on the middle 6-membered ring, and Carbon atom No. 2 is fused with the carbon atom far away from Y on the middle 6-membered ring;
more preferably, Ring A2 is

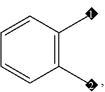

wherein Carbon atom No. 1 is fused with the carbon atom adjacent to Y on the middle 6-membered ring, and Carbon atom No. 2 is fused with the carbon atom far away from Y on the middle 6-membered ring;
Y is selected from —O—, —S—, —CR^{a}R^{b}—;
R^{a} and R^{b} are each independently selected from hydrogen and C₁-C₆ alkyl;
preferably, R^{a} and R^{b} are each independently selected from hydrogen and methyl;

preferably, Y is selected from —O—, —S—, —CH₂—,

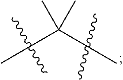 [SMILES: *C(*)(C)C]

more preferably, Y is selected from —O—, —S—;
L¹ and L² are each independently selected from the group consisting of a direct bond, phenylene, naphthylene, biphenylene, and the phenylene, naphthylene, and biphenylene are each independently optionally substituted by one R^{L};
preferably, L¹ and L² are each independently selected from the group consisting of a direct bond, phenylene, naphthylene, and biphenylene, and the phenylene is optionally substituted by one R^{L};
more preferably, L¹ and L² are each independently selected from the group consisting of a direct bond,

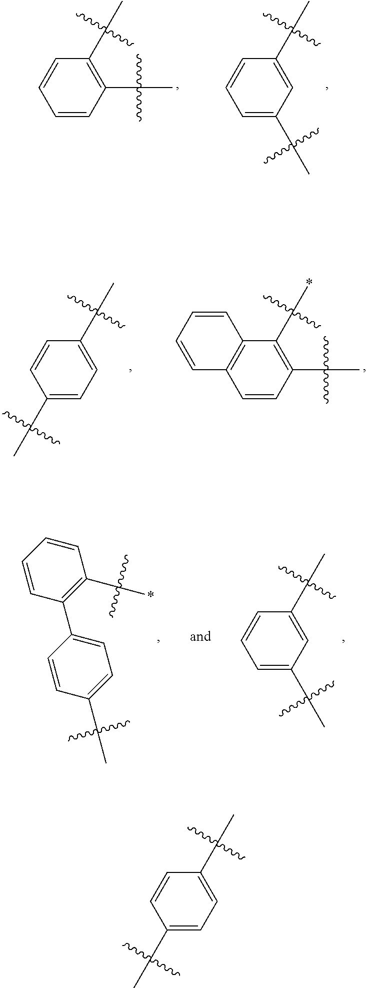

are each independently optionally substituted by one R^{L}, wherein the bond with marker * is bonded to the parent ring, and the bond without marker * is bonded to R¹ or R²;
R^{L} is selected from benzoxazolyl and benzothiazolyl;
preferably, R^{L} is benzoxazolyl;

more preferably, R^{L} is

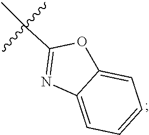 [SMILES: *c1nc2ccccc2o1]

most preferably, L¹ and L² are each independently selected from the group consisting of a direct bond,

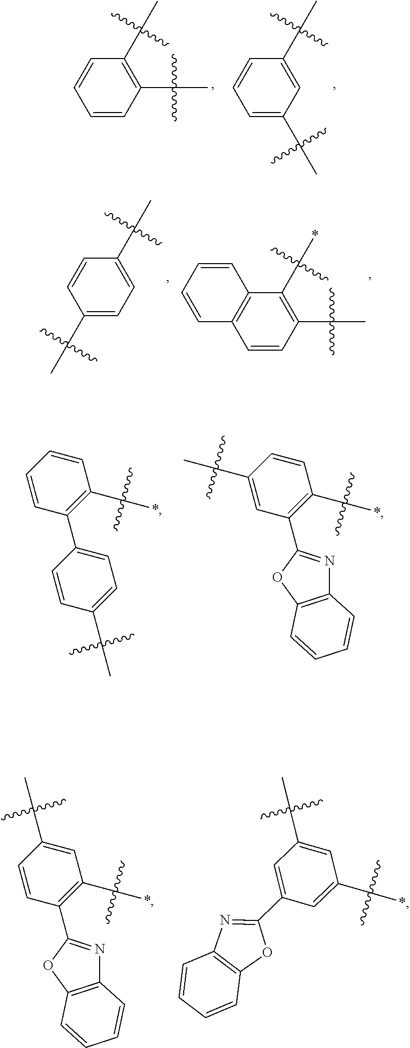

wherein the bond with marker * is bonded to the parent ring, and the bond without marker * is bonded to R¹ or R²;
R¹ and R² are each independently selected from the group consisting of diazaphenanthrolinyl, pyridinyl, pyrimidinyl, triazinyl, benzoxazolyl, benzothiazolyl, and the diazaphenanthrolinyl, pyridinyl, pyrimidinyl, triazinyl, benzoxazolyl and benzothiazolyl are each independently optionally substituted by one or two (preferably two) R^{R};
preferably, R¹ and R² are each independently selected from the group consisting of diazaphenanthrolinyl, pyridinyl, pyrimidinyl, triazinyl, benzoxazolyl, benzothiazolyl, and the pyridinyl, pyrimidinyl, triazinyl are each independently optionally substituted by one or two (preferably two) R^{R};
more preferably, R¹ and R² are each independently selected from the group consisting of o-diazaphenanthrolinyl, pyridinyl, pyrimidinyl, 1,3,5-triazinyl, and benzoxazolyl, and the pyridinyl, pyrimidinyl, and 1,3,5-triazinyl are each independently optionally substituted by one or two (preferably two) R^{R};
further preferably, R¹ and R² are each independently selected from the group consisting of

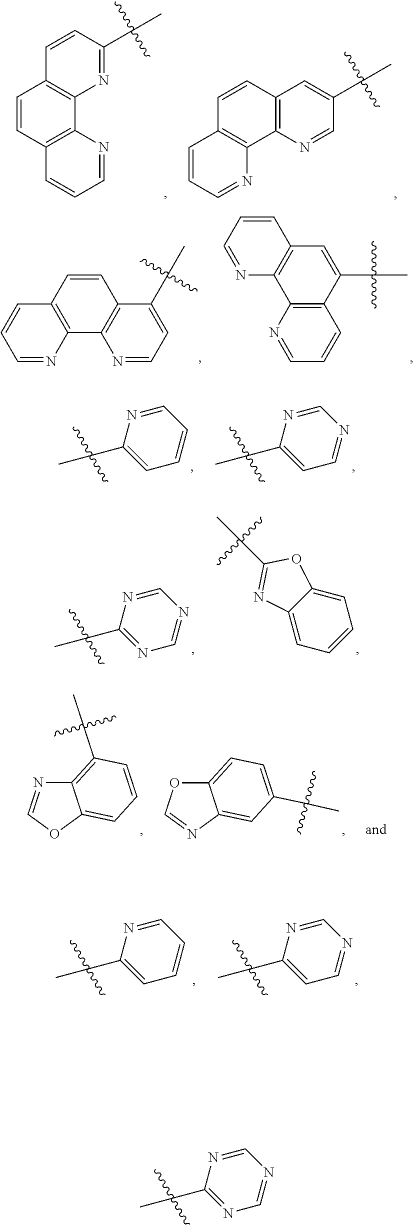

are each independently optionally substituted by one or two (preferably two) R^{R};
R^{R} is selected from the group consisting of phenyl, biphenyl, naphthyl, and fluorenyl, and the phenyl, biphenyl, naphthyl, and fluorenyl are each independently optionally substituted by two C₁-C₆ alkyl groups;
preferably, R^{R} is selected from the group consisting of phenyl, biphenyl, naphthyl, and fluorenyl, and the fluorenyl is optionally substituted by two C₁-C₆ alkyl groups;
more preferably, R^{R} is selected from the group consisting of phenyl, biphenyl, naphthyl, fluorenyl, and the fluorenyl is optionally substituted by two methyl groups;
most preferably, R^{R} is selected from the group consisting of

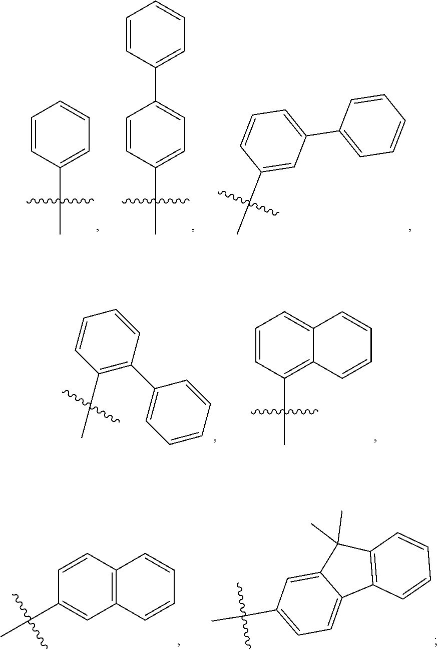

most preferably, R¹ and R² are each independently selected from the group consisting of:

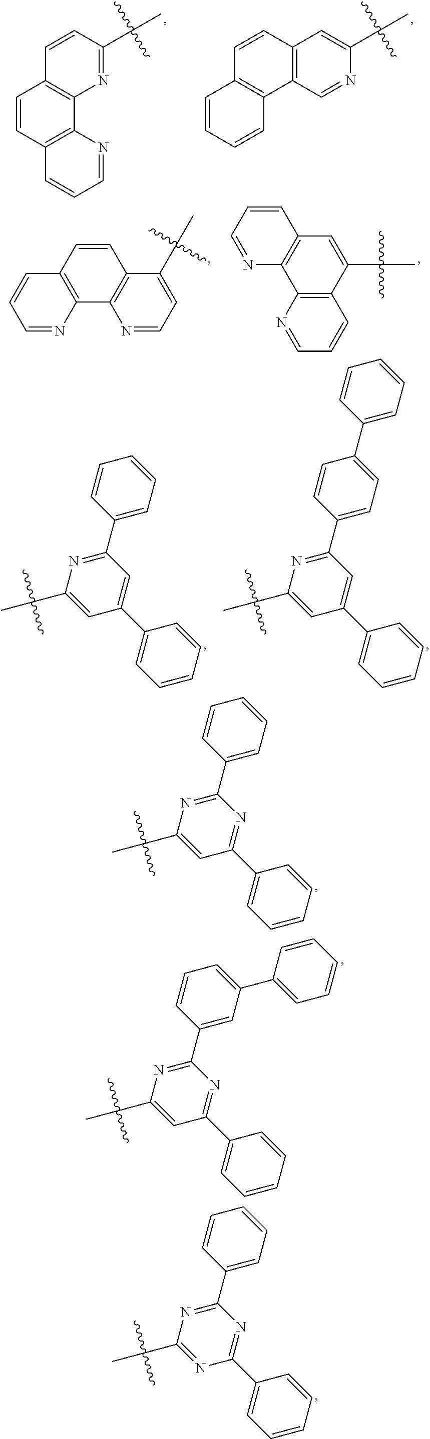

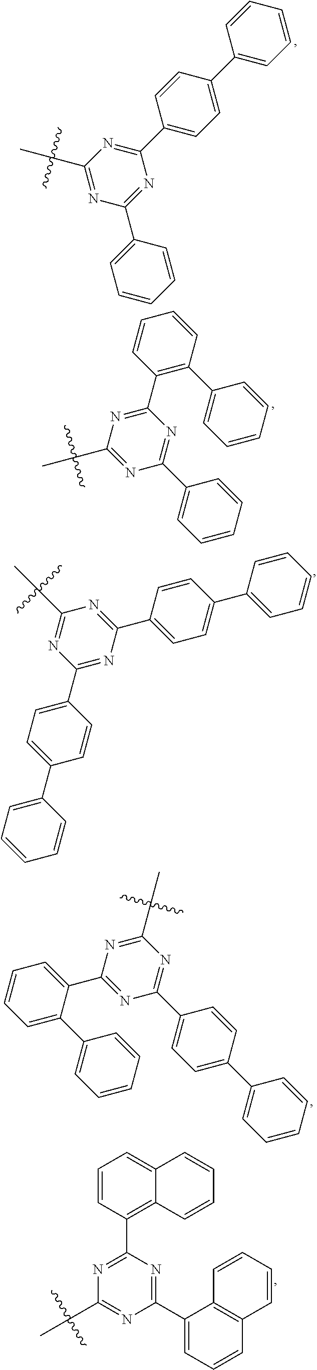

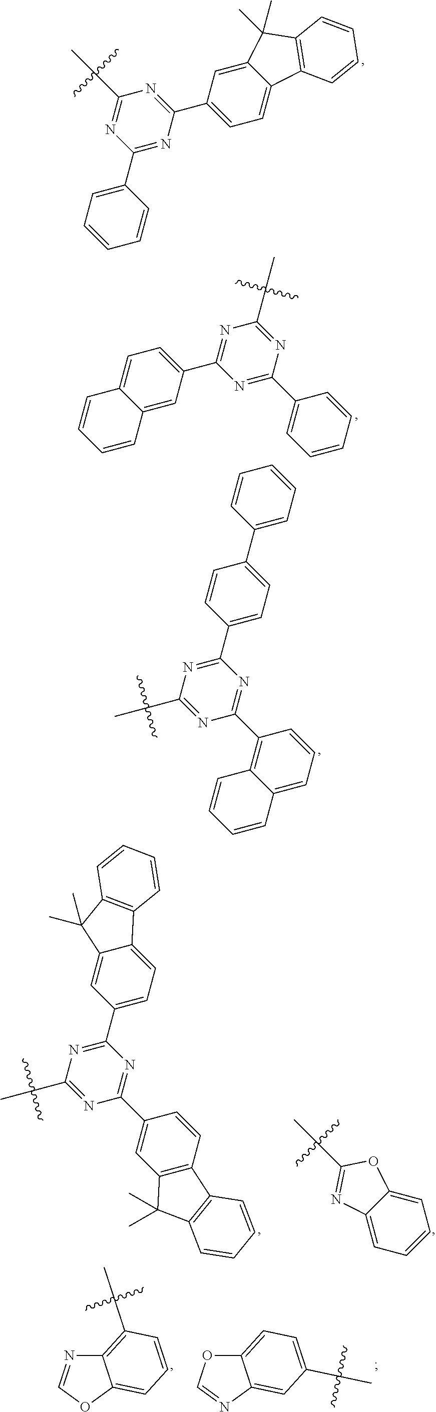

most preferably, R¹-L¹-, R²-L²- are each independently selected from the group consisting of:

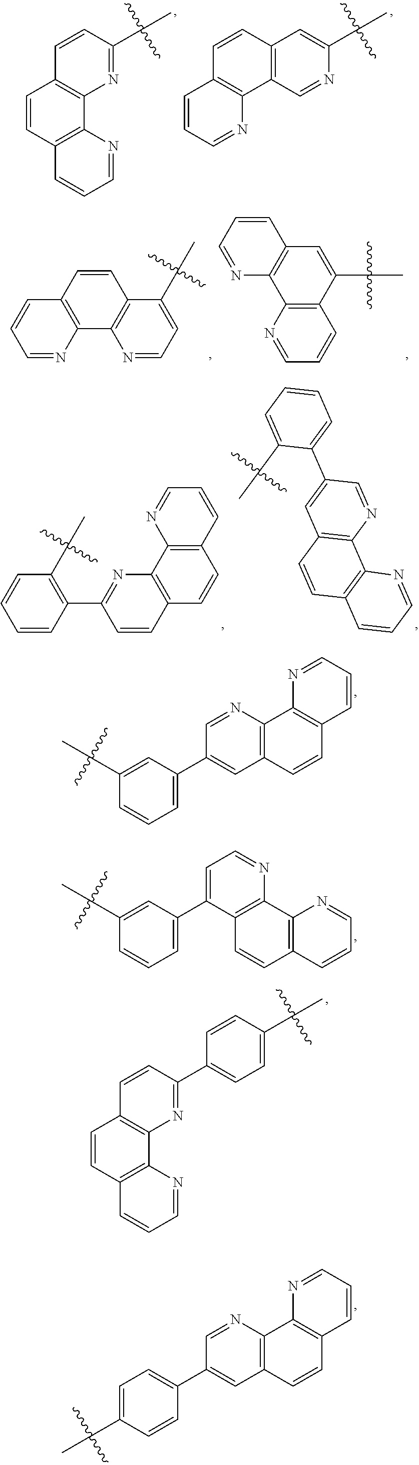

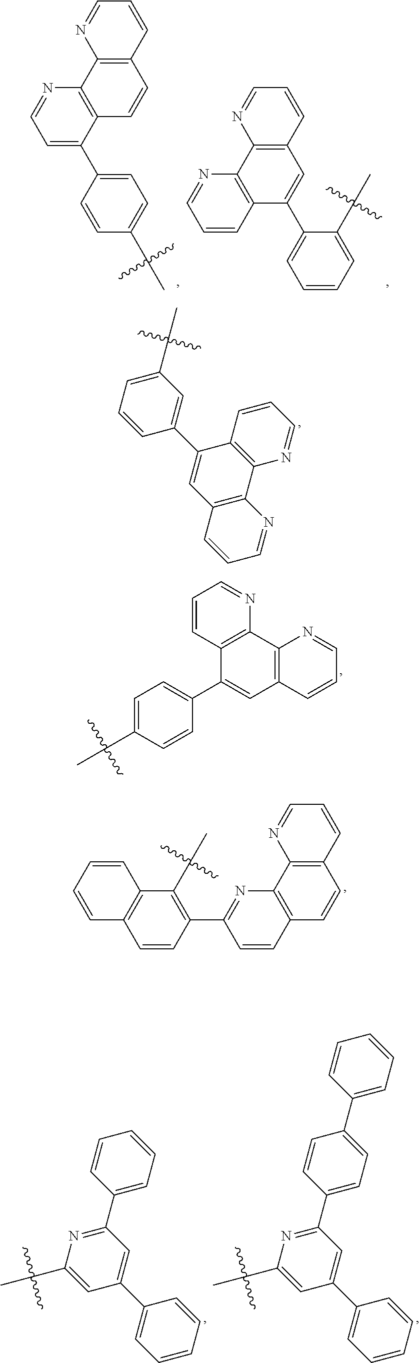

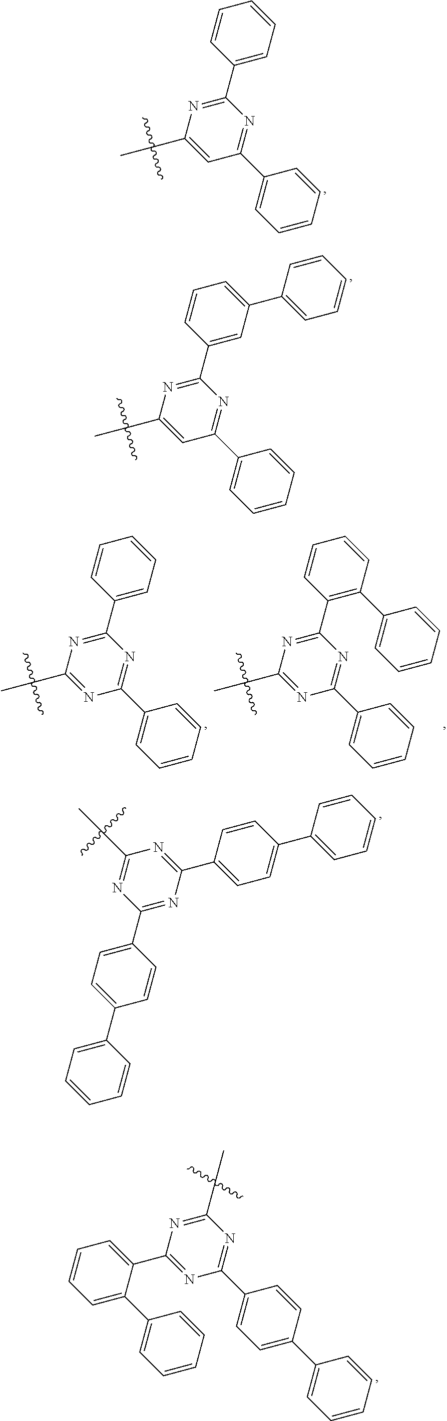

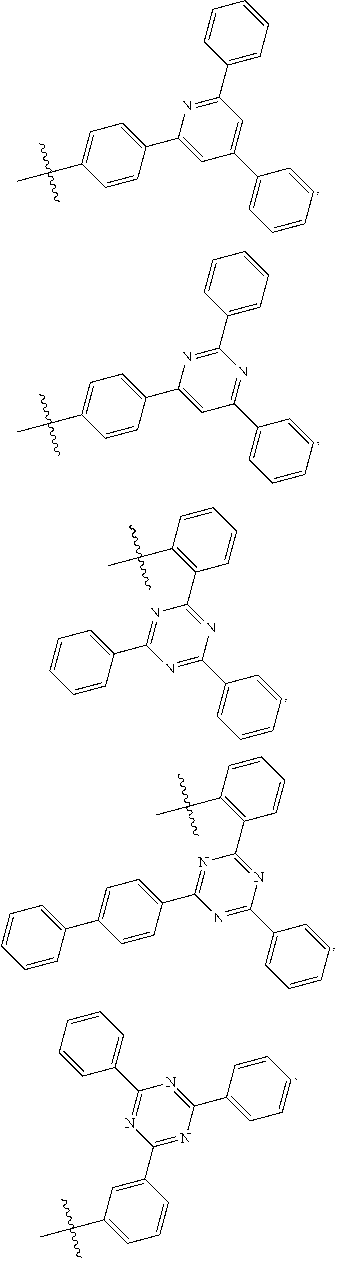

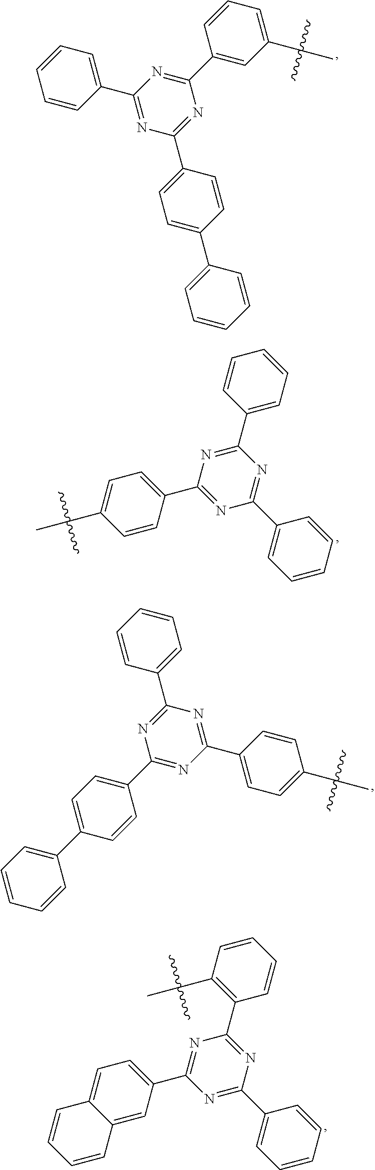

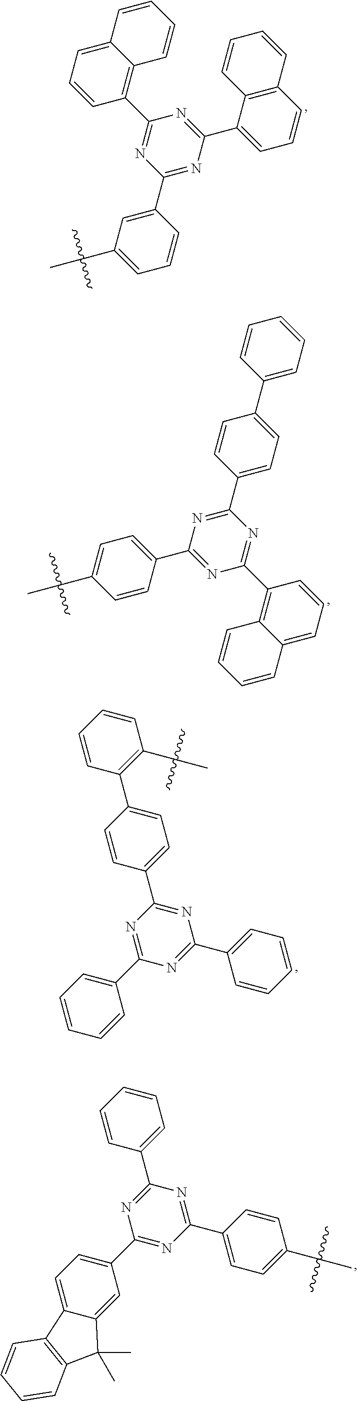 [SMILES: *c1cccc(-c2nc(-c3cccc4ccccc34)nc(-c3cccc4ccccc34)n2)c1 ; *c1ccc(-c2nc(-c3ccc(-c4ccccc4)cc3)nc(-c3cccc4ccccc34)n2)cc1 ; *c1ccccc1-c1ccc(-c2nc(-c3ccccc3)nc(-c3ccccc3)n2)cc1 ; *c1ccc(-c2nc(-c3ccccc3)nc(-c3ccc4c(c3)C(C)(C)c3ccccc3-4)n2)cc1]

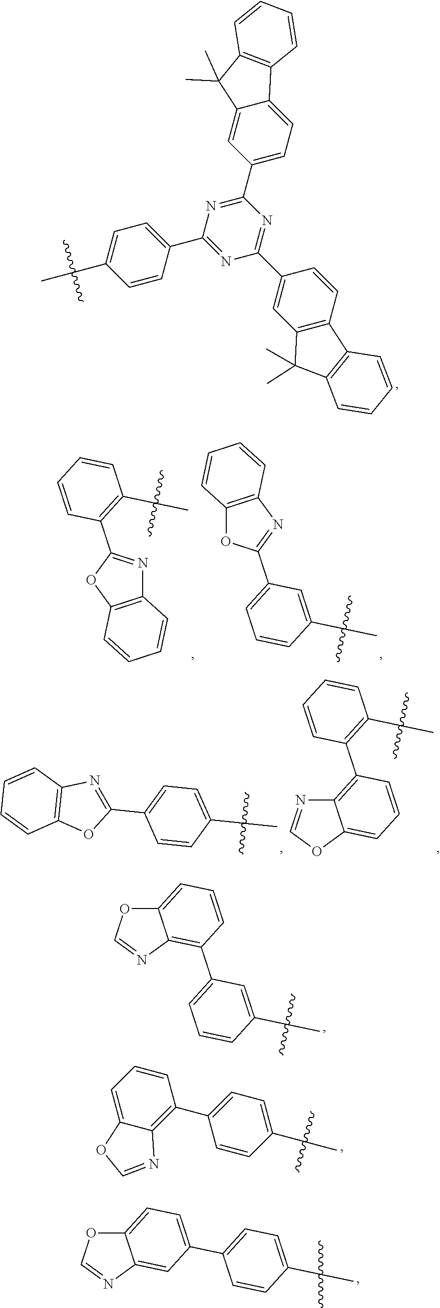

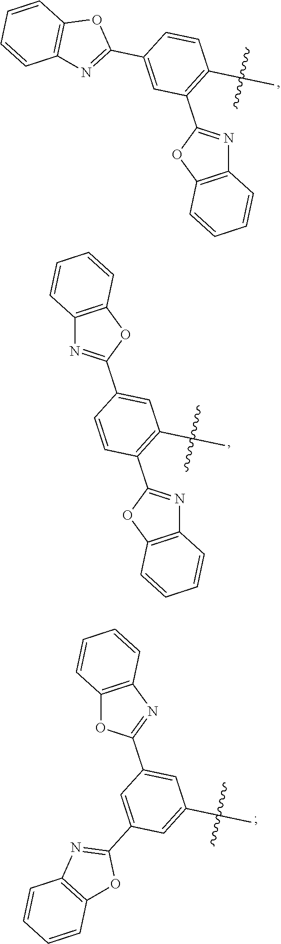 [SMILES: *c1ccc(-c2nc3ccccc3o2)cc1-c1nc2ccccc2o1 ; *c1cc(-c2nc3ccccc3o2)ccc1-c1nc2ccccc2o1 ; *c1cc(-c2nc3ccccc3o2)cc(-c2nc3ccccc3o2)c1]

R³ and R⁴ are each independently selected from hydrogen and C₁-C₆ alkyl;
preferably, R³ and R⁴ are hydrogen.

In the third aspect of the present disclosure, there is provided a compound represented by Formula I-1, or a stereoisomer or tautomer of the compound,

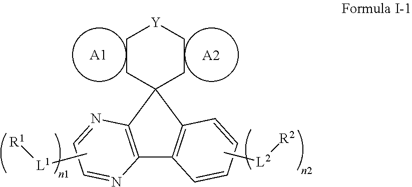

wherein:
Ring A1 is selected from benzene ring and naphthalene ring;
preferably, Ring A1 is selected from the group consisting of

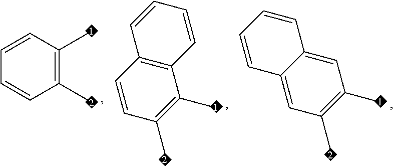

wherein Carbon atom No. 1 is fused with the carbon atom adjacent to Y on the middle 6-membered ring, and Carbon atom No. 2 is fused with the carbon atom far away from Y on the middle 6-membered ring;
more preferably, Ring A1 is

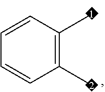

wherein Carbon atom No. 1 is fused with the carbon atom adjacent to Y on the middle 6-membered ring, and Carbon atom No. 2 is fused with the carbon atom far away from Y on the middle 6-membered ring;
Ring A2 is selected from benzene ring and naphthalene ring;
preferably, Ring A2 is selected from the group consisting of

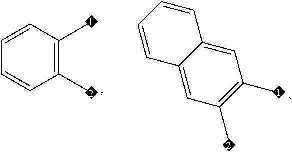

wherein Carbon atom No. 1 is fused with the carbon atom adjacent to Y on the middle 6-membered ring, and Carbon atom No. 2 is fused with the carbon atom far away from Y on the middle 6-membered ring;
more preferably, Ring A2 is

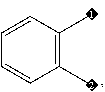

wherein Carbon atom No. 1 is fused with the carbon atom adjacent to Y on the middle 6-membered ring, and Carbon atom No. 2 is fused with the carbon atom far away from Y on the middle 6-membered ring;
Y is selected from —O—, —S—, —CR^{a}R^{b}—;
R^{a} and R^{b} are each independently selected from hydrogen and C₁-C₆ alkyl;

preferably, R^{a} and R^{b} are each independently selected from hydrogen and methyl;

preferably, Y is selected from —O—, —S—, —CH₂—,

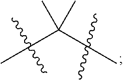 [SMILES: *C(*)(C)C]

more preferably, Y is selected from —O—, —S—;
n1 and n2 are each independently selected from 0 and 1, and n1 and n2 are not 0 at the same time;

preferably,

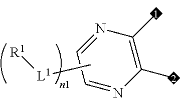

is selected from

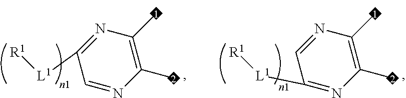

wherein Carbon atom No. 1 is bonded to the central spiro carbon atom, and Carbon atom No. 2 is bonded to a carbon atom of the aromatic ring on the right;

preferably,

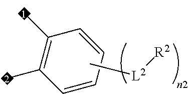

is selected from the group consisting of

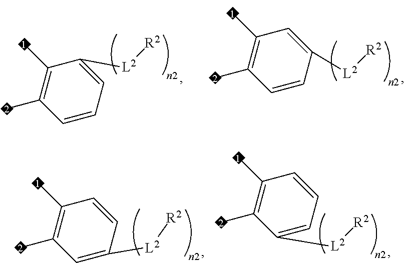

wherein Carbon atom No. 1 is bonded to the central spiro carbon atom, and Carbon atom No. 2 is bonded to a carbon atom of the aromatic ring on the left;
L¹ and L² are each independently selected from the group consisting of a direct bond, phenylene, naphthylene, and biphenylene, and the phenylene, naphthylene, and biphenylene are each independently optionally substituted by one R^{L};
preferably, L¹ and L² are each independently selected from the group consisting of a direct bond, phenylene, naphthylene, and biphenylene, and the phenylene is optionally substituted by one R^{L};
more preferably, L¹ and L² are each independently selected from the group consisting of a direct bond,

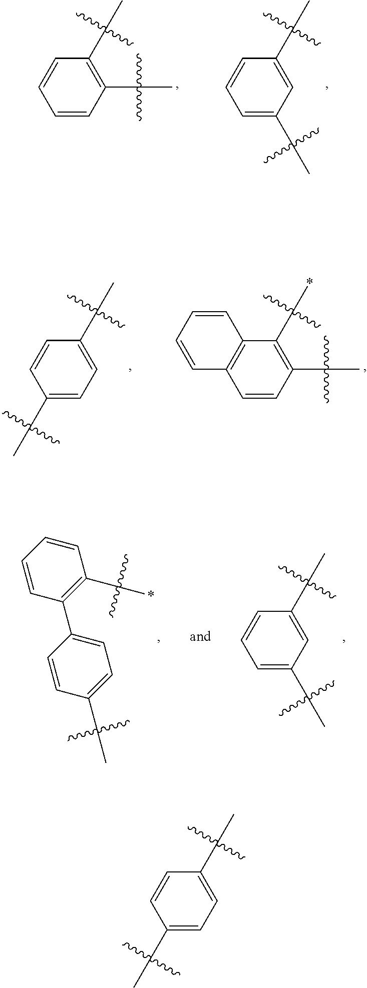

are each independently optionally substituted by one R^{L}, wherein the bond with marker * is bonded to the parent ring, and the bond without marker * is bonded to R¹ or R²,
R^{L} is selected from benzoxazolyl and benzothiazolyl;
preferably, R^{L} is benzoxazolyl;
more preferably, R^{L} is

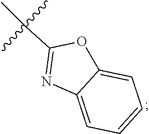 [SMILES: *c1nc2ccccc2o1]

most preferably, L¹ and L² are each independently selected from the group consisting of a direct bond,

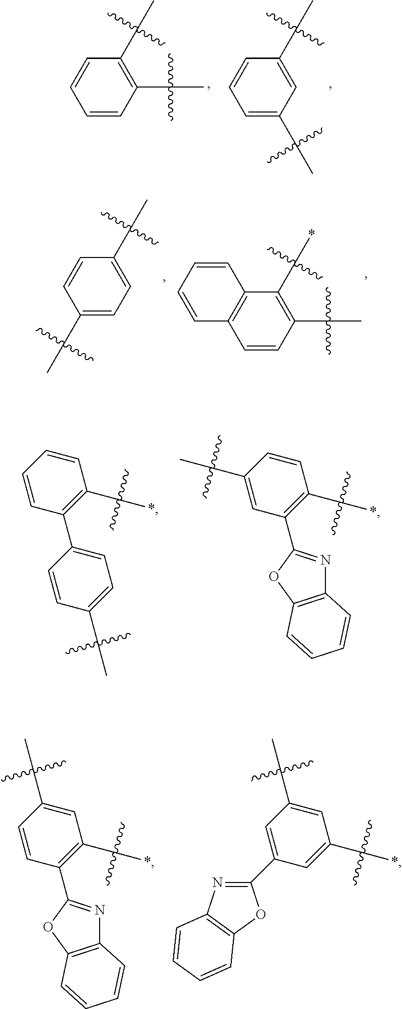

wherein the bond with marker * is bonded to the parent ring, and the bond without marker * is bonded to R¹ or R²;
R¹ and R² are each independently selected from the group consisting of diazaphenanthrolinyl, pyridinyl, pyrimidinyl, triazinyl, benzoxazolyl, benzothiazolyl, and the diazaphenanthrolinyl, pyridinyl, pyrimidinyl, triazinyl, benzoxazolyl and benzothiazolyl are each independently optionally substituted by one or two (preferably two) R^{R};
preferably, R¹ and R² are each independently selected from the group consisting of diazaphenanthrolinyl, pyridinyl, pyrimidinyl, triazinyl, benzoxazolyl, benzothiazolyl, and the pyridinyl, pyrimidinyl, triazinyl are each independently optionally substituted by one or two (preferably two) R^{R};
more preferably, R¹ and R² are each independently selected from the group consisting of o-diazaphenanthrolinyl, pyridinyl, pyrimidinyl, 1,3,5-triazinyl, and benzoxazolyl, and the pyridinyl, pyrimidinyl, and 1,3,5-triazinyl are each independently optionally substituted by one or two (preferably two) R^{R};
further preferably, R¹ and R² are each independently selected from the group consisting of

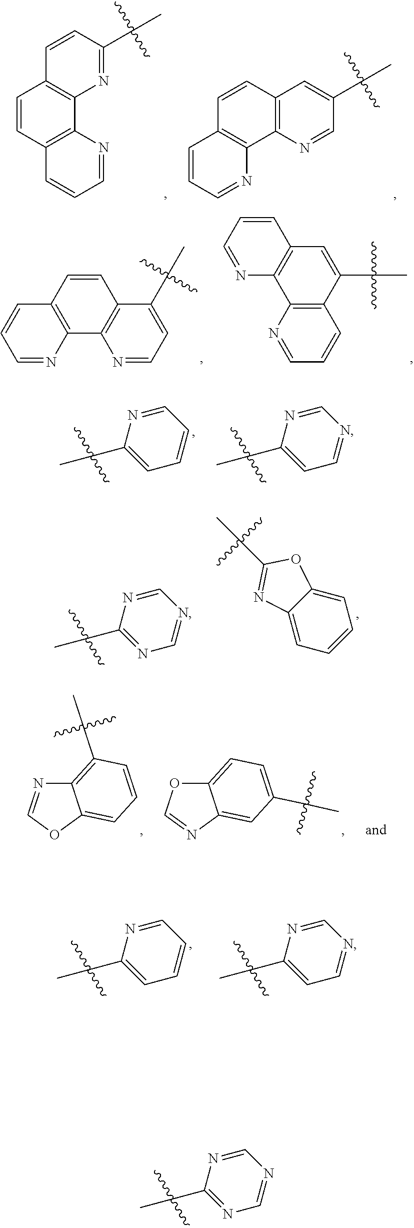

are each independently optionally substituted by one or two (preferably two) R^{R};
R^{R} is selected from the group consisting of phenyl, biphenyl, naphthyl, and fluorenyl, and the phenyl, biphenyl, naphthyl, and fluorenyl are each independently optionally substituted by two C₁-C₆ alkyl groups;
preferably, R^{R} is selected from the group consisting of phenyl, biphenyl, naphthyl, and fluorenyl, and the fluorenyl is optionally substituted by two C₁-C₆ alkyl groups;
more preferably, R^{R} is selected from the group consisting of phenyl, biphenyl, naphthyl, and fluorenyl, and the fluorenyl is optionally substituted by two methyl groups;
most preferably, R^{R} is selected from the group consisting of

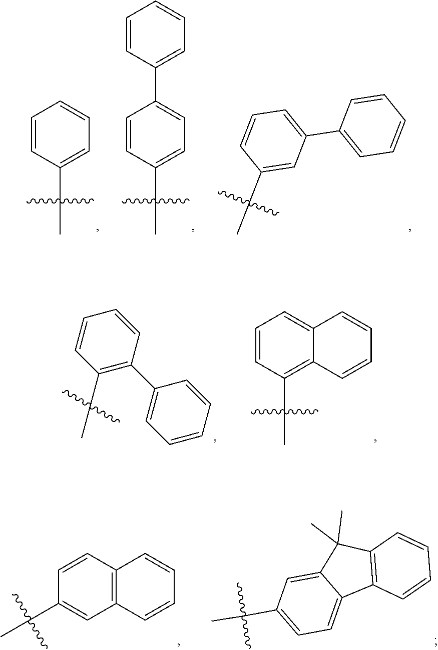

most preferably, R¹ and R² are each independently selected from the group consisting of:

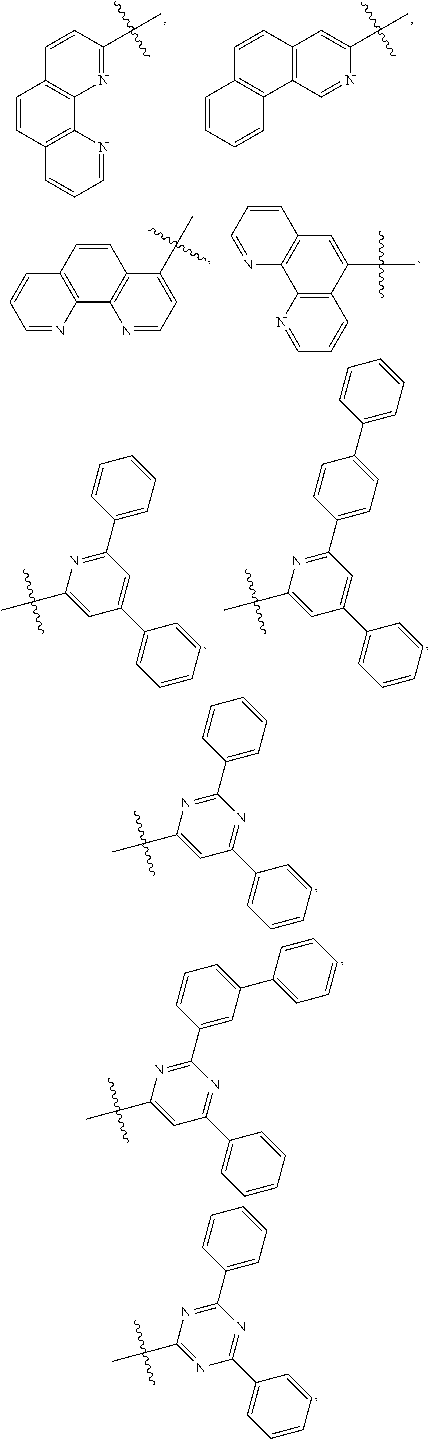

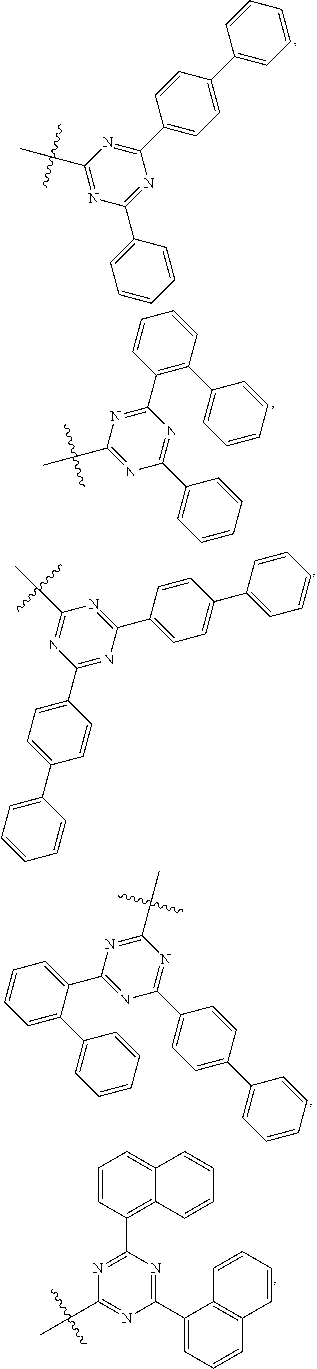

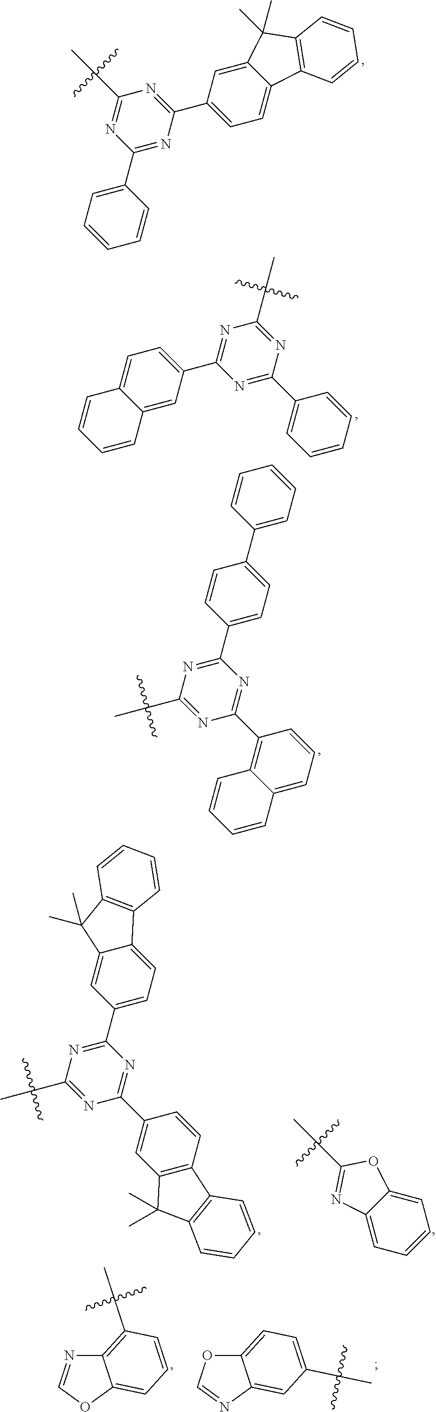

most preferably, R¹-L¹-, R²-L²- are each independently selected from the group consisting of:

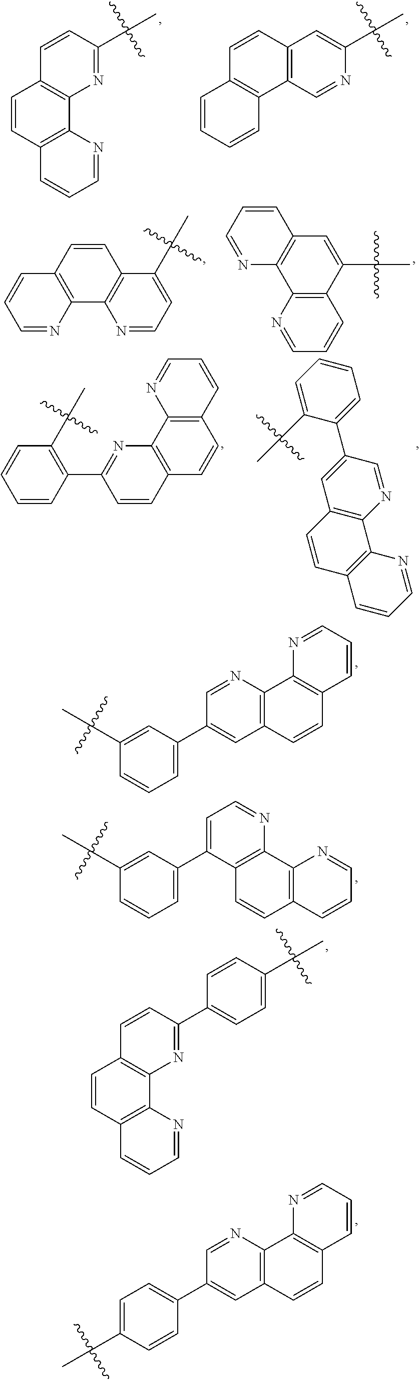

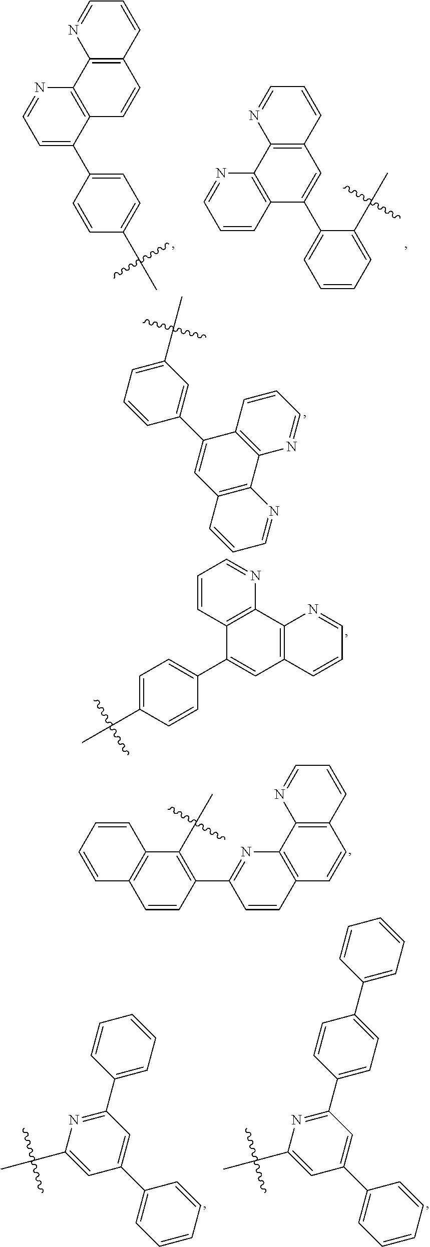

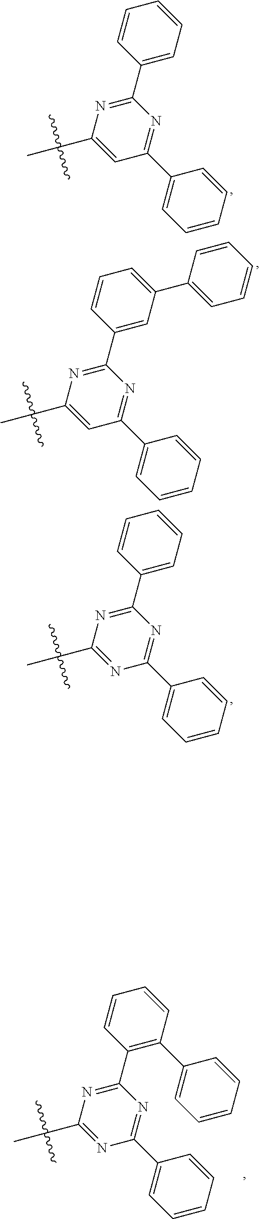

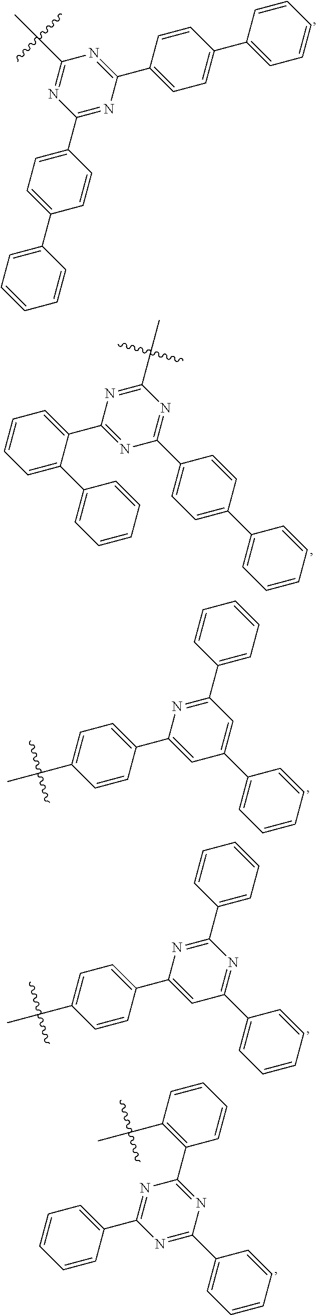

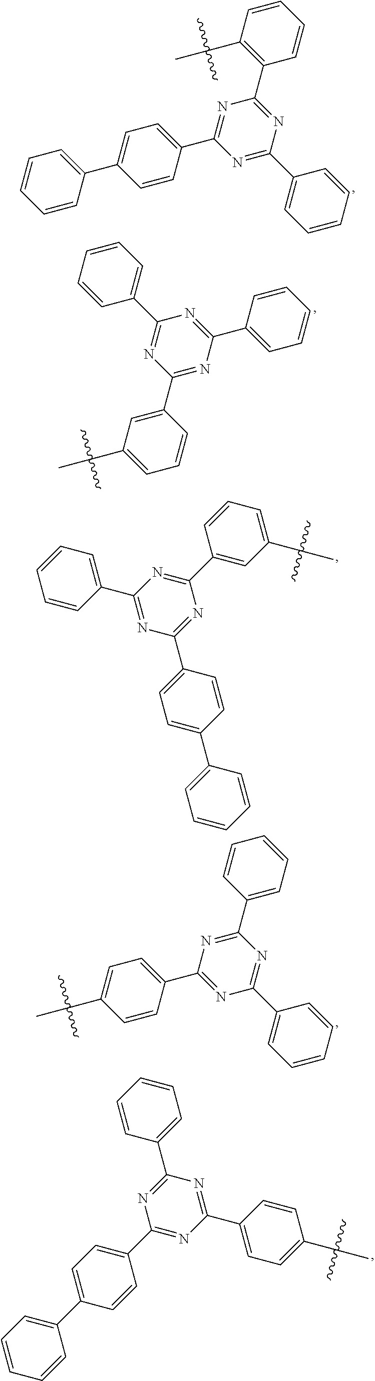

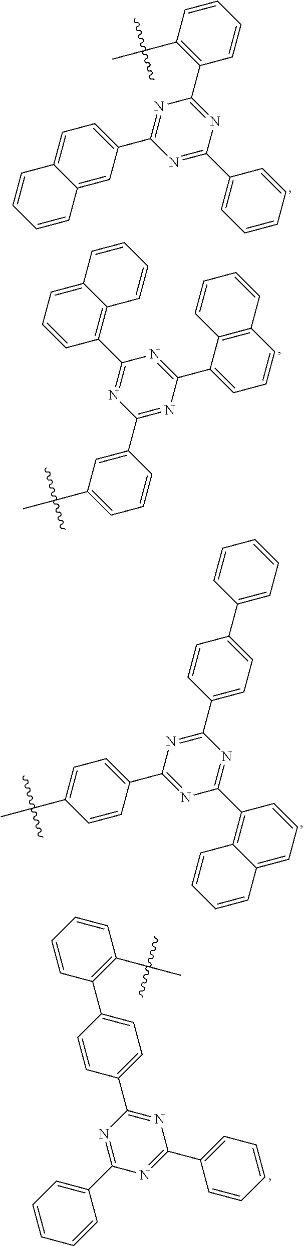

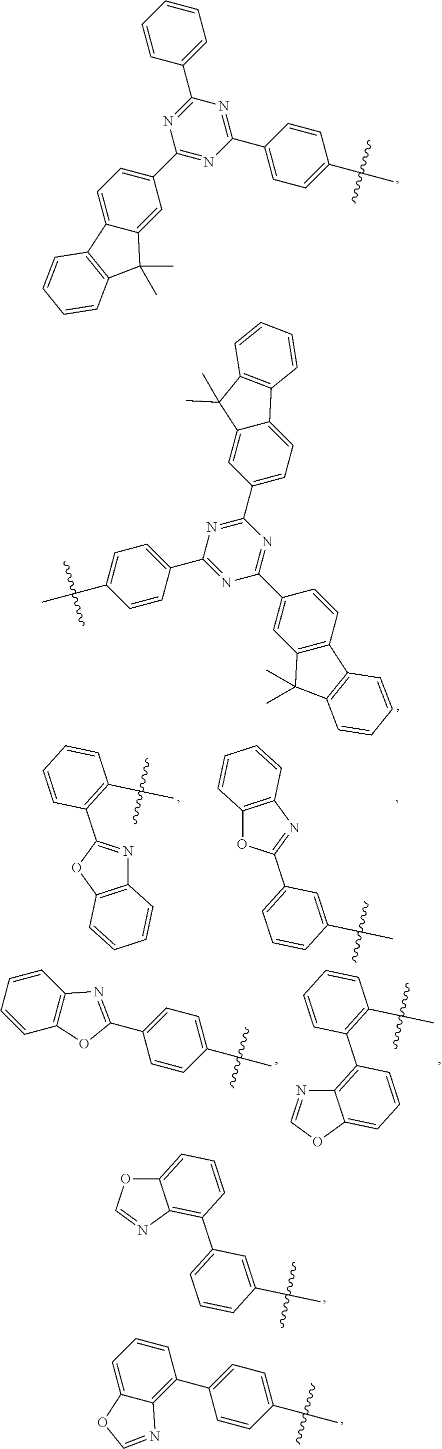

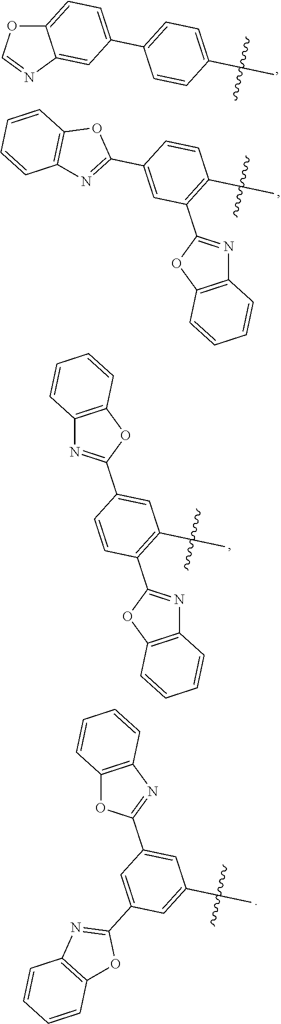

In the fourth aspect of the present disclosure, there is provided a compound represented by Formula I-2, or a stereoisomer or tautomer of the compound,

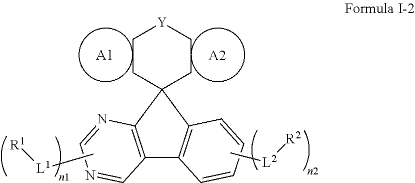

wherein:
Ring A1 is selected from benzene ring and naphthalene ring;
preferably, Ring A1 is selected from the group consisting of

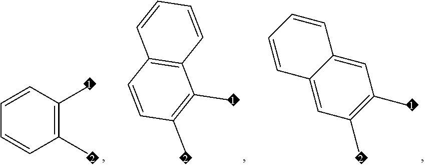

wherein Carbon atom No. 1 is fused with the carbon atom adjacent to Y on the middle 6-membered ring, and Carbon atom No. 2 is fused with the carbon atom far away from Y on the middle 6-membered ring;
more preferably, Ring A1 is

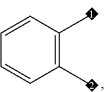

wherein Carbon atom No. 1 is fused with the carbon atom adjacent to Y on the middle 6-membered ring, and Carbon atom No. 2 is fused with the carbon atom far away from Y on the middle 6-membered ring;
Ring A2 is selected from benzene ring and naphthalene ring;
preferably, Ring A2 is selected from the group consisting of

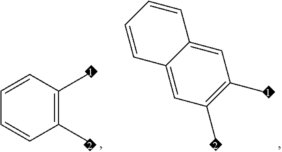

wherein Carbon atom No. 1 is fused with the carbon atom adjacent to Y on the middle 6-membered ring, and Carbon atom No. 2 is fused with the carbon atom far away from Y on the middle 6-membered ring;
more preferably, Ring A2 is

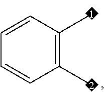

wherein Carbon atom No. 1 is fused with the carbon atom adjacent to Y on the middle 6-membered ring, and Carbon atom No. 2 is fused with the carbon atom far away from Y on the middle 6-membered ring;
Y is selected from —O—, —S—, —CR^{a}R^{b}—;
R^{a} and R^{b} are each independently selected from hydrogen and C₁-C₆ alkyl;
preferably, R^{a} and R^{b} are each independently selected from hydrogen and methyl;
preferably, Y is selected from —O—, —S—, —CH₂,

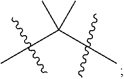 [SMILES: *C(*)(C)C]

more preferably, Y is selected from —O—, —S—;
n1 and n2 are each independently selected from 0 and 1, and n1 and n2 are not 0 at the same time;
preferably, n1 is 0 and n2 is 1;
preferably,

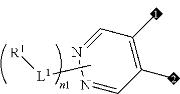

is selected from the group consisting of

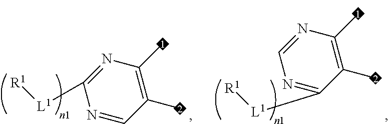

wherein Carbon atom No. 1 is bonded to the central spiro carbon atom, and Carbon atom No. 2 is bonded to a carbon atom of the aromatic ring on the right;
preferably,

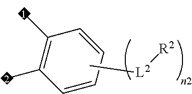

is selected from the group consisting of

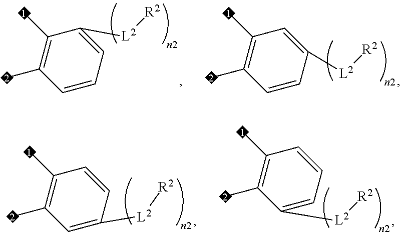

wherein Carbon atom No. 1 is bonded to the central spiro carbon atom, and Carbon atom No. 2 is bonded to a carbon atom of the aromatic ring on the left;
L¹ and L² are each independently selected from the group consisting of a direct bond and phenylene;
preferably, L¹ and L² are direct bonds;
R¹ and R² are each independently selected from diazaphenanthrolinyl, and the diazaphenanthrolinyl is optionally substituted by one or two (preferably two) R^{R}, and R^{R} is phenyl;
preferably, R¹ and R² are each independently selected from diazaphenanthrolinyl;
more preferably, R¹ and R² are each independently selected from the group consisting of:

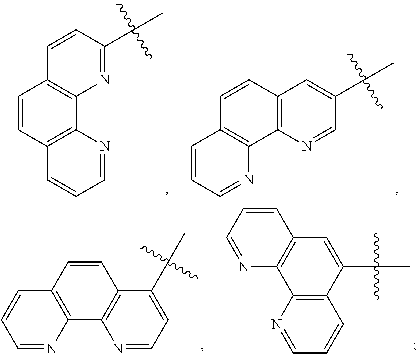

most preferably, R¹-L¹-, R²-L²- are each independently selected from the group consisting of:

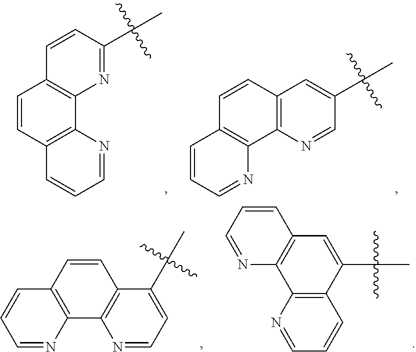

In the fifth aspect of the present disclosure, there is provided a compound represented by Formula I-3, or a stereoisomer or tautomer of the compound,

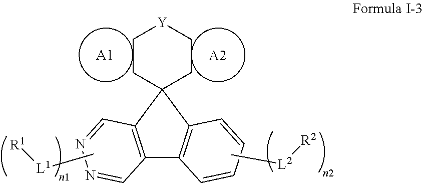

wherein:
Ring A1 is selected from benzene ring and naphthalene ring;
preferably, Ring A1 is selected from the group consisting of

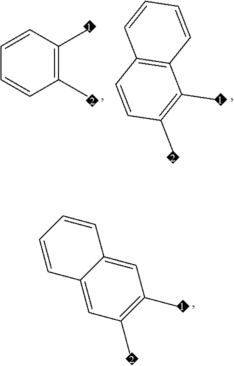

wherein Carbon atom No. 1 is fused with the carbon atom adjacent to Y on the middle 6-membered ring, and Carbon atom No. 2 is fused with the carbon atom far away from Y on the middle 6-membered ring;
more preferably, Ring A1 is

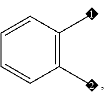

wherein Carbon atom No. 1 is fused with the carbon atom adjacent to Y on the middle 6-membered ring, and Carbon atom No. 2 is fused with the carbon atom far away from Y on the middle 6-membered ring;
Ring A2 is selected from benzene ring and naphthalene ring;
preferably, Ring A2 is selected from the group consisting of

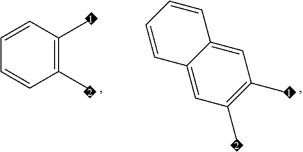

wherein Carbon atom No. 1 is fused with the carbon atom adjacent to Y on the middle 6-membered ring, and Carbon atom No. 2 is fused with the carbon atom far away from Y on the middle 6-membered ring;
more preferably, Ring A2 is

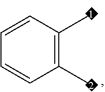

wherein Carbon atom No. 1 is fused with the carbon atom adjacent to Y on the middle 6-membered ring, and Carbon atom No. 2 is fused with the carbon atom far away from Y on the middle 6-membered ring;
Y is selected from —O—, —S—, —CR^{a}R^{b}—;
R^{a} and R^{b} are each independently selected from hydrogen and C₁-C₆ alkyl;
preferably, R^{a} and R^{E} are each independently selected from hydrogen and methyl;
preferably, Y is selected from —O—, —S—, —CH₂—,

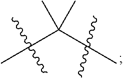 [SMILES: *C(*)(C)C]

more preferably, Y is selected from —O—, —S—;

n1 and n2 are each independently selected from 0 and 1, and n1 and n2 are not 0 at the same time;
preferably, n1 is 0 and n2 is 1;
preferably,

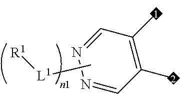

is selected from

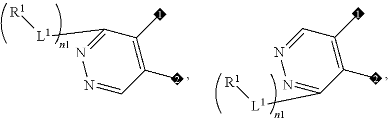

wherein Carbon atom No. 1 is bonded to the central spiro carbon atom, and Carbon atom No. 2 is bonded to a carbon atom of the aromatic ring on the right;
preferably,

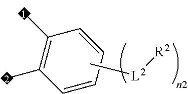

is selected from the group consisting of

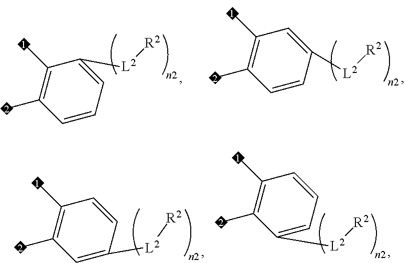

wherein Carbon atom No. 1 is bonded to the central spiro carbon atom, and Carbon atom No. 2 is bonded to a carbon atom of the aromatic ring on the left;
L¹ and L² are each independently selected from the group consisting of a direct bond and phenylene;
preferably, L¹ and L² are direct bonds;
R¹ and R² are each independently selected from the group consisting of diazaphenanthrolinyl, and the diazaphenanthrolinyl is optionally substituted by one or two (preferably two) R^{R}, and R^{R} is phenyl;
preferably, R¹ and R² are each independently selected from diazaphenanthrolinyl;
more preferably, R¹ and R² are each independently selected from the group consisting of:

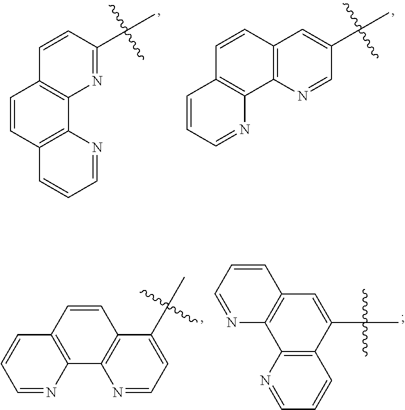 [SMILES: *c1ccc2ccc3cccnc3c2n1 ; *c1cnc2c(ccc3cccnc32)c1 ; *c1ccnc2c1ccc1cccnc12 ; *c1cc2cccnc2c2ncccc12]

most preferably, R¹-L¹-, R²-L²- are each independently selected from the group consisting of:

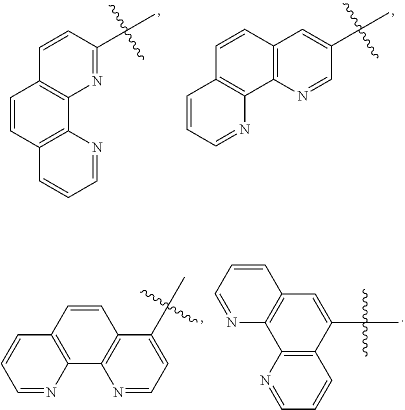 [SMILES: *c1ccc2ccc3cccnc3c2n1 ; *c1cnc2c(ccc3cccnc32)c1 ; *c1ccnc2c1ccc1cccnc12 ; *c1cc2cccnc2c2ncccc12]

In the sixth aspect of the present disclosure, there is provided a compound represented by Formula II, or a stereoisomer or tautomer of the compound,

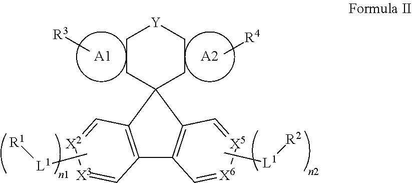

X² and X³ are each independently selected from CH and N, and at least one of X² and X³ is N;
X⁵ and X⁶ are each independently selected from CH and N, and at least one of X⁵ and X⁶ is N;
n1 and n2 are each independently selected from 0 and 1, and n1 and n2 are not 0 at the same time;
preferably,

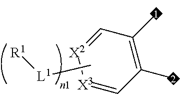

is selected from the group consisting of

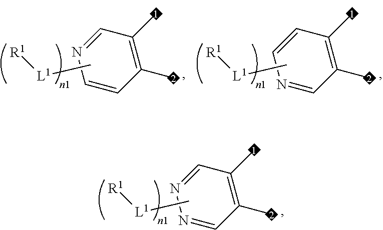

wherein Carbon atom No. 1 is bonded to the central spiro carbon atom, and Carbon atom No. 2 is bonded to a carbon atom of the aromatic ring on the right;
more preferably,

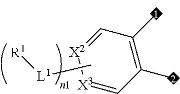

is selected from the group consisting of:

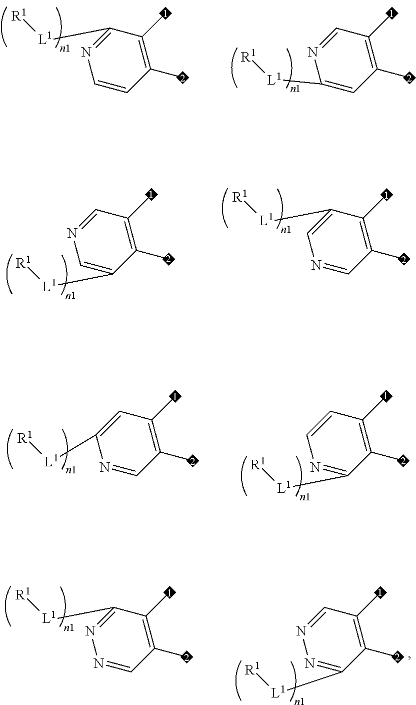

wherein, Carbon atom No. 1 is bonded to the central spiro carbon atom, and Carbon atom No. 2 is bonded to a carbon atom of the aromatic ring on the right;
preferably,

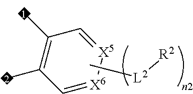

is selected from

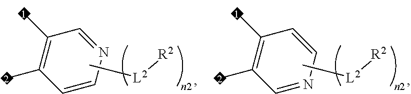

wherein Carbon atom No. 1 is bonded to the central spiro carbon atom, and Carbon atom No. 2 is bonded to a carbon atom of the aromatic ring on the left;
more preferably,

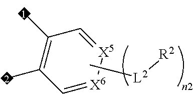

is selected from the group consisting of:

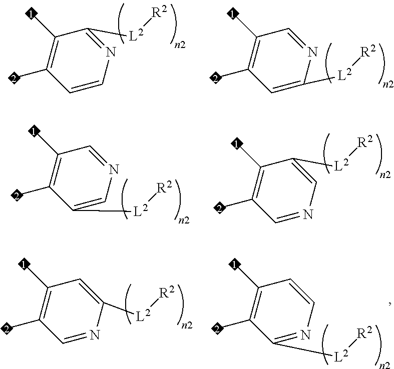

wherein, Carbon atom No. 1 is bonded to the central spiro carbon atom, and Carbon atom No. 2 is bonded to a carbon atom of the aromatic ring on the left;
Ring A1 and Ring A2 are each independently selected from benzene ring and naphthalene ring;
preferably, Ring A1 and Ring A2 are benzene rings;
Y is selected from —O—, —S—, —CR^{a}R^{b}—;
R^{a} and R^{b} are each independently selected from hydrogen and C₁-C₆ alkyl;
preferably, R^{a} and R^{b} are each independently selected from C₁-C₆ alkyl;
more preferably, R^{a} and R^{b} are methyl groups;
preferably, Y is selected from —O—, —S—,

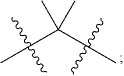 [SMILES: *C(*)(C)C]

more preferably, Y is selected from —O—, —S—;
L¹ and L² are each independently selected from the group consisting of a direct bond, phenylene, and naphthylene, and the phenylene and naphthylene are each independently optionally substituted by one R^{L};
preferably, L¹ and L² are each independently selected from the group consisting of a direct bond and phenylene, and the phenylene is optionally substituted by one R^{L};
more preferably, L¹ and L² are each independently selected from the group consisting of a direct bond,

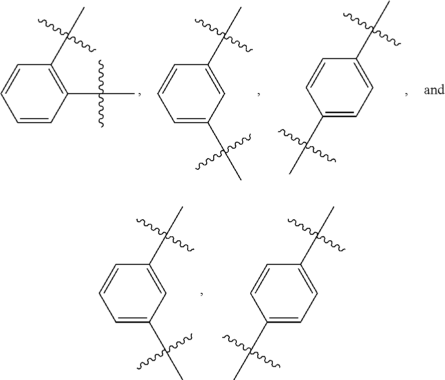 [SMILES: *c1ccccc1* ; *c1cccc(*)c1 ; *c1ccc(*)cc1 ; *c1cccc(*)c1 ; *c1ccc(*)cc1]

are each independently optionally substituted by one R^{L};
R^{L} is selected from benzoxazolyl and benzothiazolyl;
preferably, R^{L} is benzoxazolyl;
more preferably, R^{L} is selected from

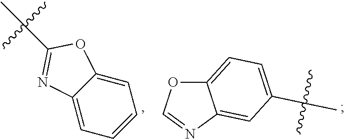

most preferably, L¹ and L² are each independently selected from the group consisting of:
a direct bond,

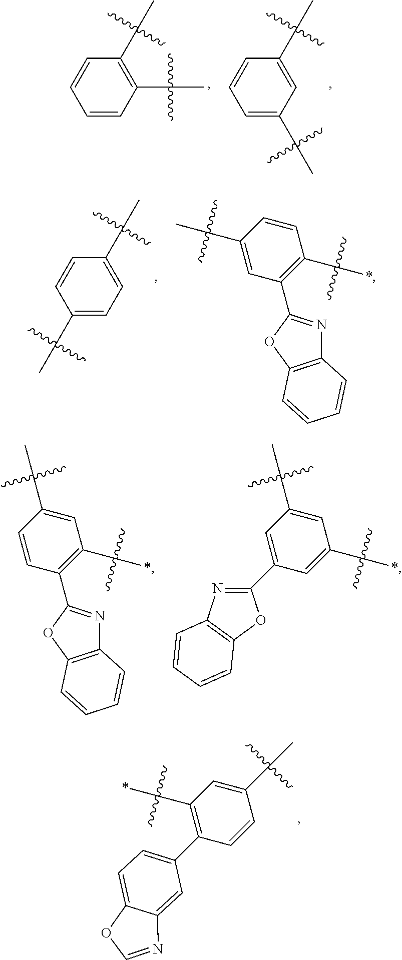

wherein, the bond with marker * is bonded to the parent ring, and the bond without marker * is bonded to R¹ or R²;
R¹ and R² are each independently selected from the group consisting of diazaphenanthrolinyl, pyridinyl, pyrimidinyl, triazinyl, benzoxazolyl, benzothiazolyl, and the diazaphenanthrolinyl, pyridinyl, pyrimidinyl, triazinyl, benzoxazolyl and benzothiazolyl are each independently optionally substituted by one or two (preferably two) R^{R};
preferably, R¹ and R² are each independently selected from the group consisting of diazaphenanthrolinyl, pyridinyl, pyrimidinyl, triazinyl, benzoxazolyl, benzothiazolyl, and the pyridinyl, pyrimidinyl, triazinyl, benzoxazolyl are each independently optionally substituted by one or two (preferably two) R^{R};
more preferably, R¹ and R² are each independently selected from o-diazaphenanthrolinyl, pyridinyl, pyrimidinyl, 1,3,5-triazinyl, benzoxazolyl, and the pyridinyl, pyrimidinyl, 1,3,5-triazinyl, benzoxazolyl are each independently optionally substituted by one or two (preferably two) R^{R};
further preferably, R¹ and R² are each independently selected from the group consisting of

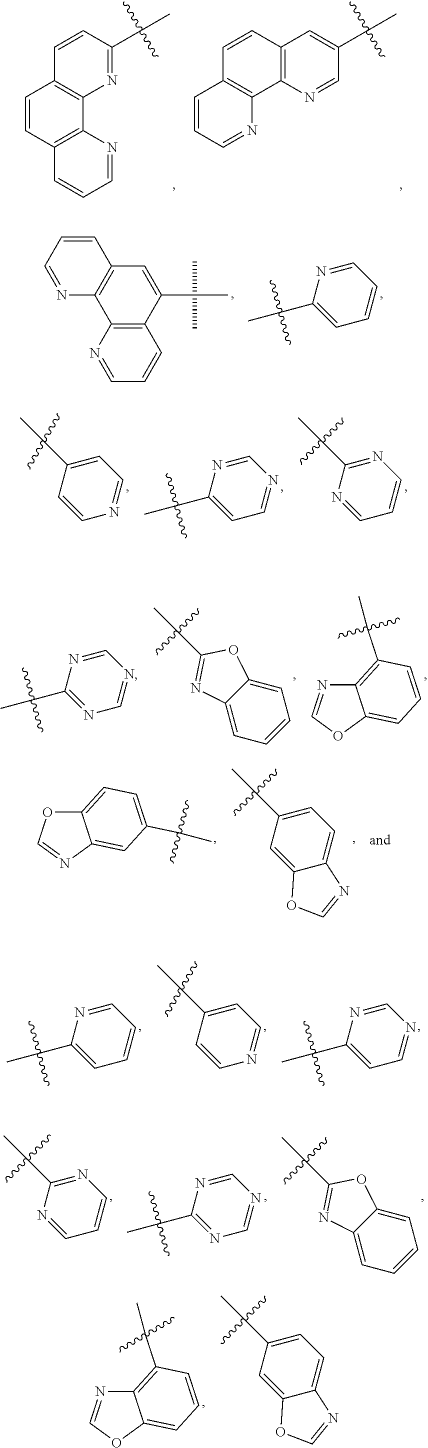

are each independently optionally substituted by one or two (preferably two) R^{R};
R^{R} is selected from the group consisting of phenyl, biphenyl, naphthyl, and fluorenyl, and the phenyl, biphenyl, naphthyl, and fluorenyl are each independently optionally substituted by two C₁-C₆ alkyl groups;
preferably, R^{R} is selected from the group consisting of phenyl, biphenyl, naphthyl, and fluorenyl, and the fluorenyl is optionally substituted by two C₁-C₆ alkyl groups;
more preferably, R^{R} is selected from the group consisting of phenyl, biphenyl, naphthyl, and fluorenyl, and the fluorenyl is optionally substituted by two methyl groups;
most preferably, R^{R} is selected from the group consisting of

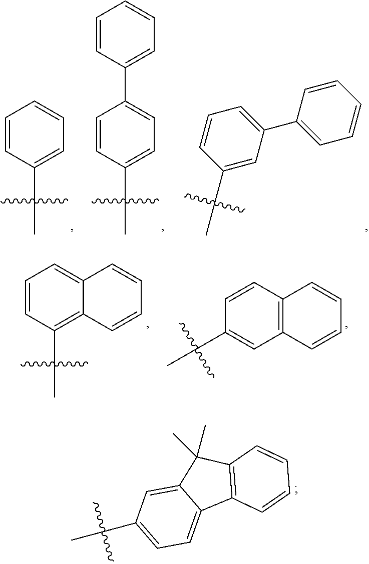

most preferably, R¹ and R² are each independently selected from the group consisting of:

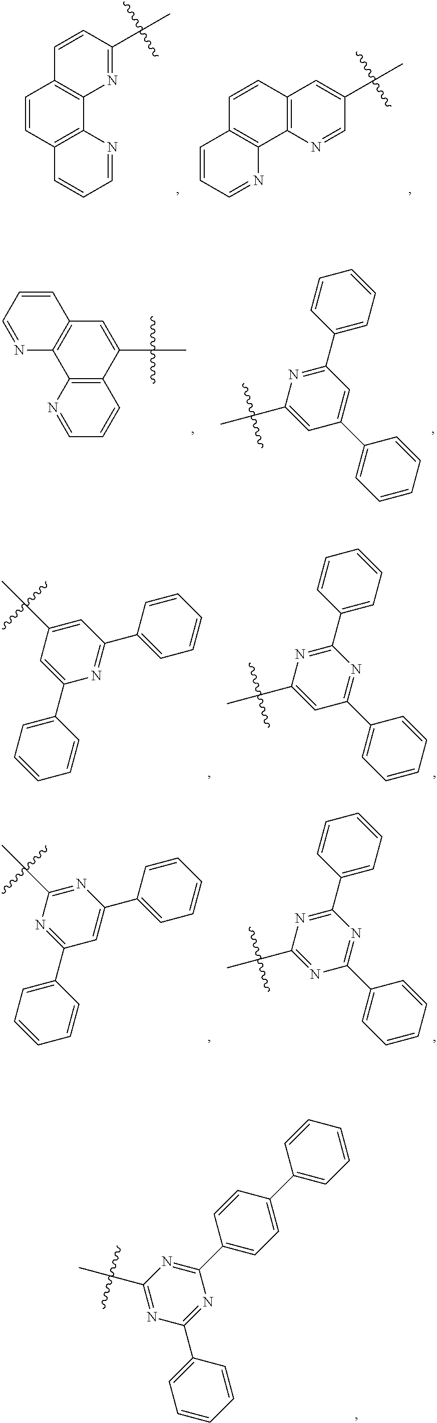

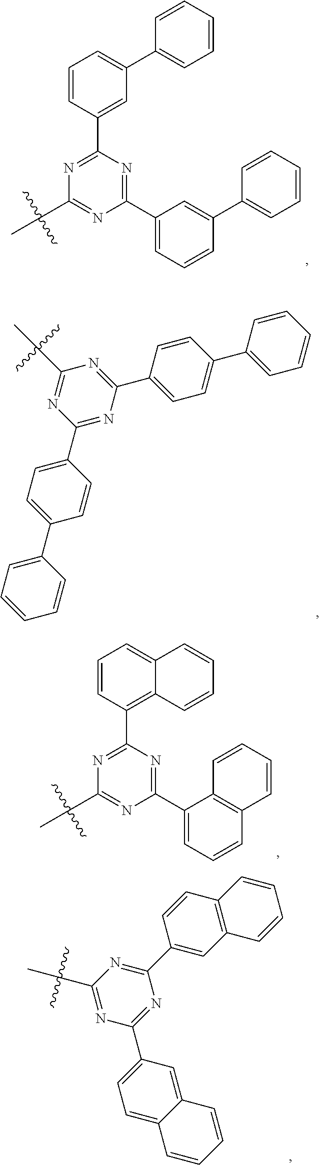

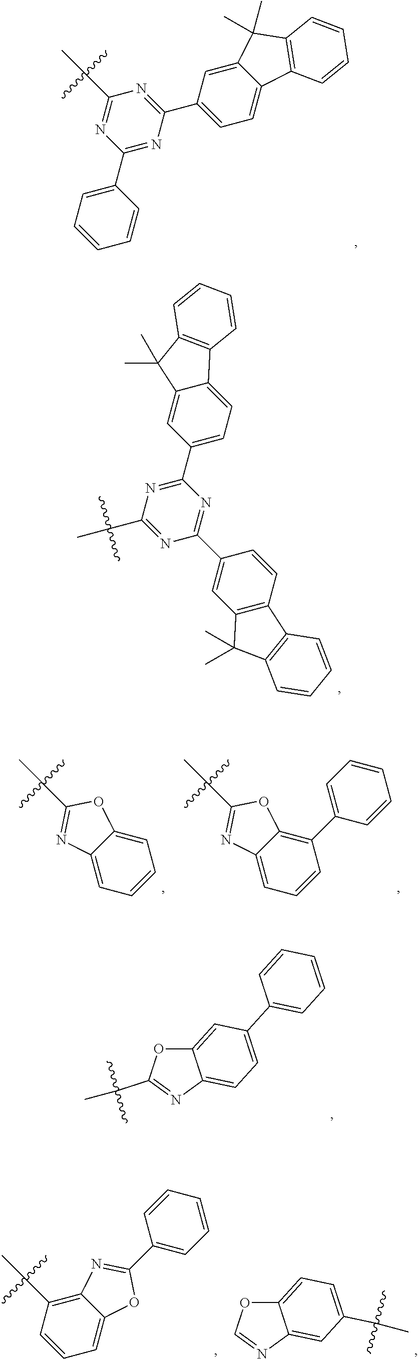

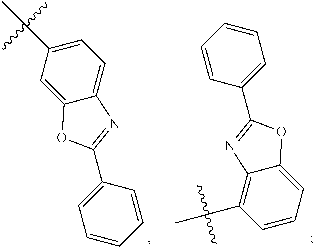 [SMILES: *c1ccc2nc(-c3ccccc3)oc2c1 ; *c1cccc2oc(-c3ccccc3)nc12]

most preferably, R¹-L¹-, R²-L²- are each independently selected from the group consisting of:

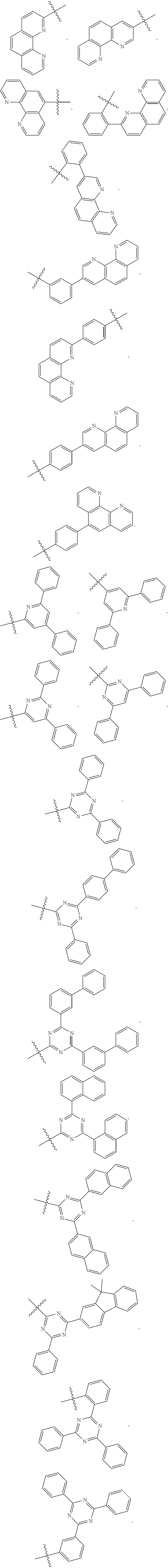

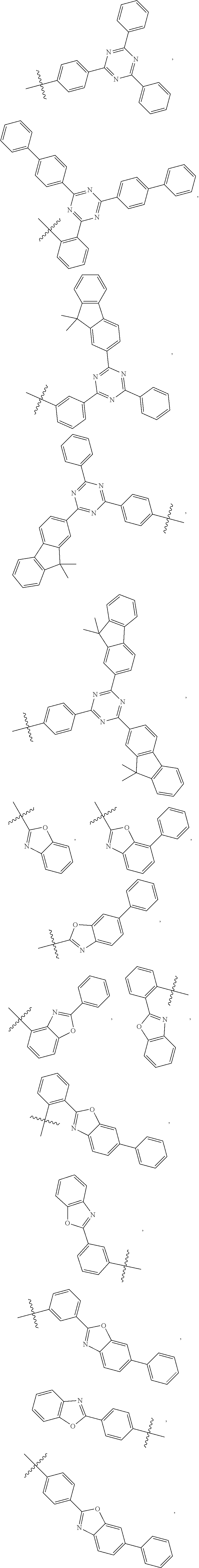

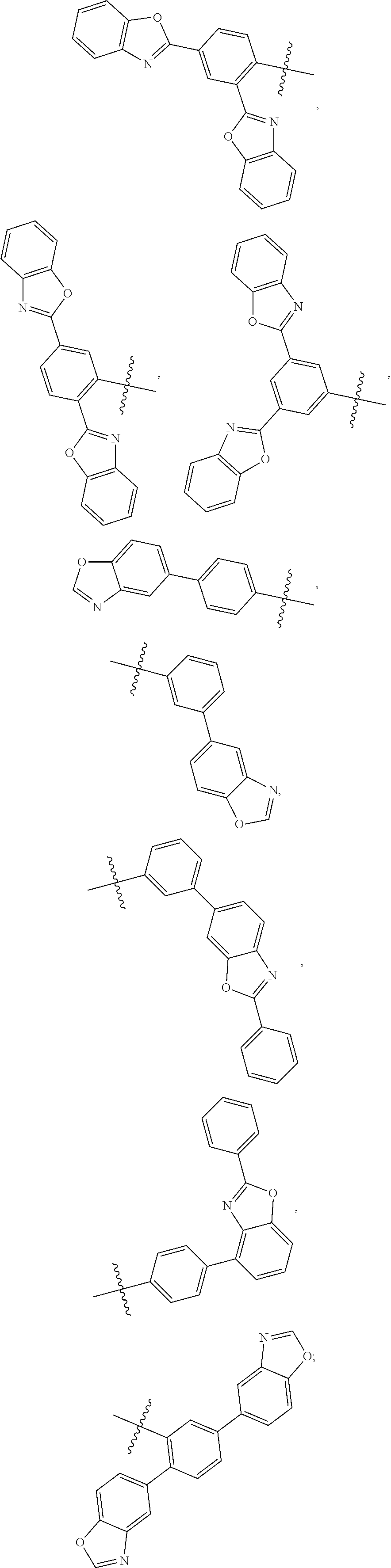

R³ and R⁴ are each independently selected from hydrogen and C₁-C₆ alkyl;
preferably, R³ and R⁴ are hydrogen.

In the seventh aspect of the present disclosure, there is provided a compound represented by Formula II-1, or a stereoisomer or tautomer of the compound,

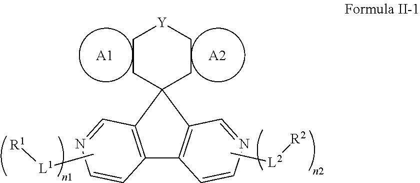

wherein:
Ring A1 and Ring A2 are each independently selected from benzene ring and naphthalene ring;
preferably, Ring A1 and Ring A2 are benzene rings;
Y is selected from —O—, —S—, —CR^{a}R^{b}—;
R^{a} and R^{b} are each independently selected from hydrogen and C₁-C₆ alkyl;
preferably, R^{a} and R^{b} are each independently selected from C₁-C₆ alkyl;
more preferably, R^{a} and R^{b} are methyl groups;
preferably, Y is selected from —O—, —S—,

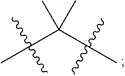 [SMILES: *C(*)(C)C]

more preferably, Y is selected from —O—, —S—;
n1 and n2 are each independently selected from 0 and 1, and n1 and n2 are not 0 at the same time;
preferably,

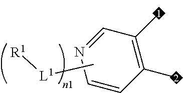

is selected from the group consisting of

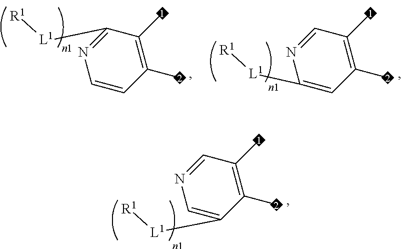

wherein Carbon atom No. 1 is bonded to the central spiro carbon atom, and Carbon atom No. 2 is bonded to a carbon atom of the aromatic ring on the right;
preferably,

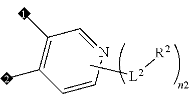

is selected from the group consisting of

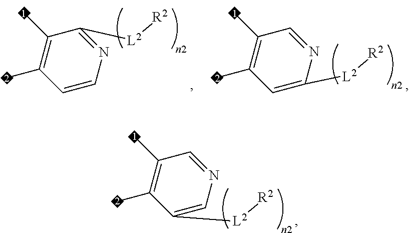

wherein, Carbon atom No. 1 is bonded to the central spiro carbon atom, and Carbon atom No. 2 is bonded to a carbon atom of the aromatic ring on the left;
L¹ and L² are each independently selected from the group consisting of a direct bond and phenylene, and the phenylene is optionally substituted by one R^{L}, and R^{L} is benzoxazolyl;
preferably, L¹ and L² are each independently selected from the group consisting of a direct bond,

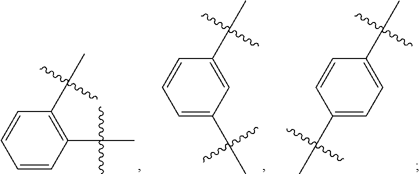 [SMILES: *c1ccccc1* ; *c1cccc(*)c1 ; *c1ccc(*)cc1]

R¹ and R² are each independently selected from the group consisting of diazaphenanthrolinyl, pyridinyl, triazinyl, benzoxazolyl, and benzothiazolyl, and the diazaphenanthrolinyl, pyridinyl, triazinyl, benzoxazolyl and benzothiazolyl are each independently optionally substituted by one or two (preferably two) R^{R};
preferably, R¹ and R² are each independently selected from the group consisting of diazaphenanthrolinyl, pyridinyl, triazinyl, and benzoxazolyl, and the pyridinyl, triazinyl, and benzoxazolyl are each independently optionally substituted by one or two (preferably two) R^{R};
more preferably, R¹ and R² are each independently selected from the group consisting of o-diazaphenanthrolinyl, pyridinyl, 1,3,5-triazinyl, and benzoxazolyl, and the pyridinyl, 1,3,5-triazinyl, and benzoxazolyl are each independently optionally substituted by one or two (preferably two) R^{R};
further preferably, R¹ and R² are each independently selected from the group consisting of

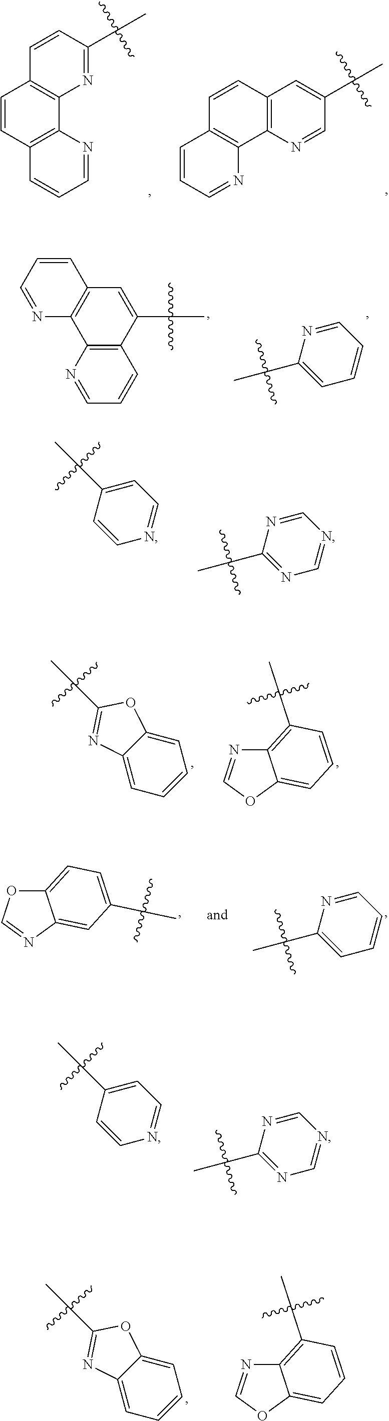

are each independently optionally substituted by one or two (preferably two) R^{R};
R^{R} is selected from the group consisting of phenyl, biphenyl, and fluorenyl, and the phenyl, biphenyl, and fluorenyl are each independently optionally substituted by two C₁-C₆ alkyl groups;
preferably, R^{R} is selected from the group consisting of phenyl, biphenyl, and fluorenyl, and the fluorenyl is optionally substituted by two C₁-C₆ alkyl groups;
more preferably, R^{R} is selected from the group consisting of phenyl, biphenyl, and fluorenyl, and the fluorenyl is optionally substituted by two methyl groups;
most preferably, R^{R} is selected from the group consisting of

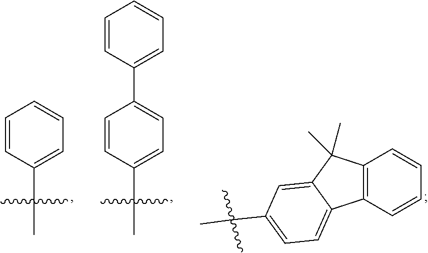

most preferably, R¹ and R² are each independently selected from the group consisting of:

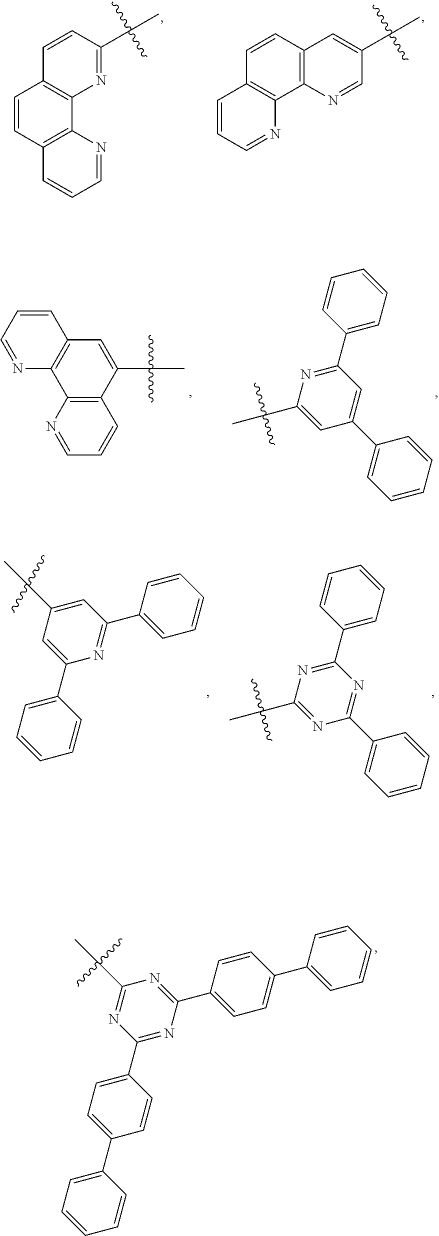

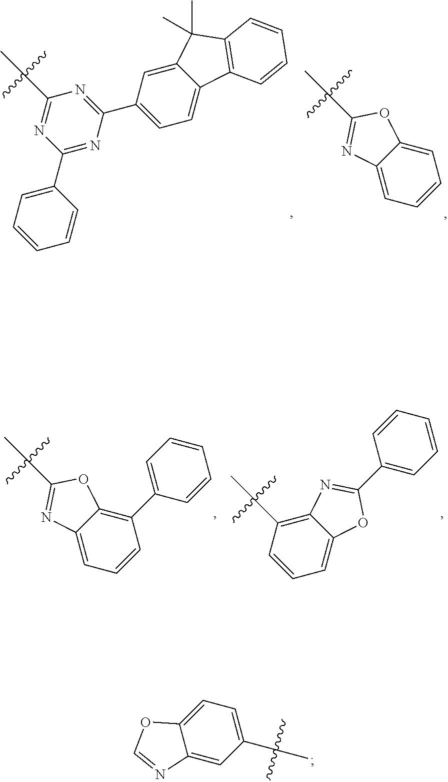 [SMILES: *c1nc(-c2ccccc2)nc(-c2ccc3c(c2)C(C)(C)c2ccccc2-3)n1 ; *c1nc2ccccc2o1 ; *c1nc2cccc(-c3ccccc3)c2o1 ; *c1cccc2oc(-c3ccccc3)nc12 ; *c1ccc2ocnc2c1]

most preferably, R¹-L¹-, R²-L²- are each independently selected from the group consisting of:

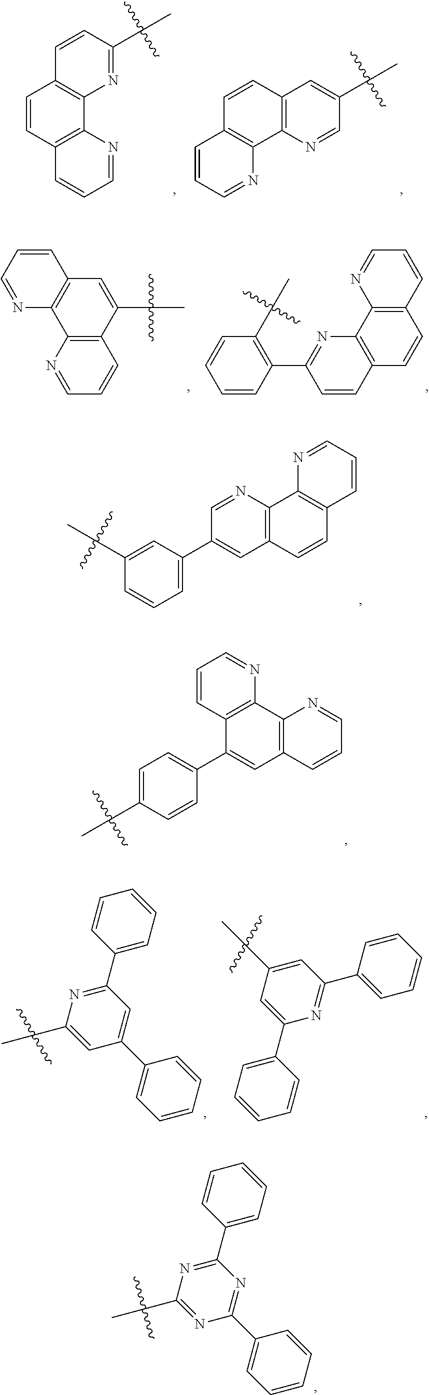

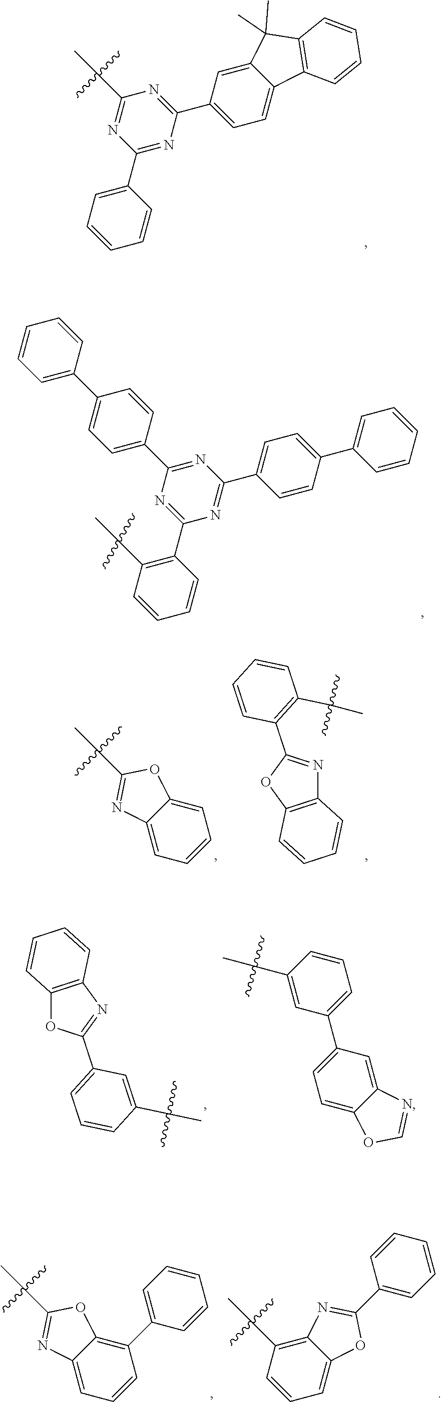

In the eighth aspect of the present disclosure, there is provided a compound represented by Formula II-2, or a stereoisomer or tautomer of the compound,

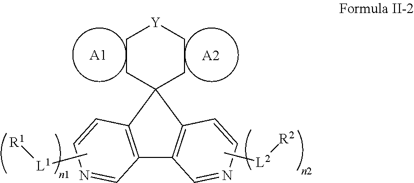

wherein:
Ring A1 and Ring A2 are each independently selected from benzene ring and naphthalene ring;
preferably, Ring A1 and Ring A2 are benzene rings;
Y is selected from —O—, —S—, —CR^{a}R^{b}—;
R^{a} and R^{b} are each independently selected from hydrogen and C₁-C₆ alkyl;
preferably, R^{a} and R^{b} are each independently selected from C₁-C₆ alkyl;
more preferably, R^{a} and R^{b} are methyl groups;
preferably, Y is selected from —O—, —S—,

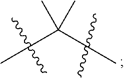 [SMILES: *C(*)(C)C]

more preferably, Y is selected from —O—, —S—;
n1 and n2 are each independently selected from 0 and 1, and n1 and n2 are not 0 at the same time;
preferably,

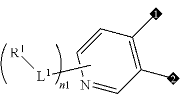

is selected from the group consisting of

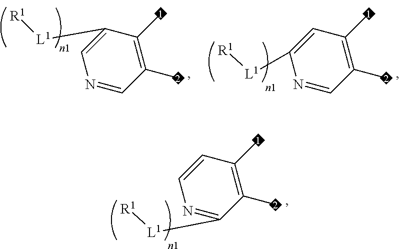

wherein Carbon atom No. 1 is bonded to the central spiro carbon atom, and Carbon atom No. 2 is bonded to a carbon atom of the aromatic ring on the right;
preferably,

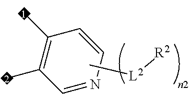

is selected from the group consisting of

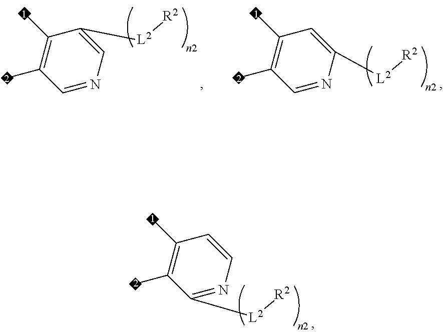

wherein, Carbon atom No. 1 is bonded to the central spiro carbon atom, and Carbon atom No. 2 is bonded to a carbon atom of the aromatic ring on the left;
L¹ and L² are each independently selected from the group consisting of a direct bond and phenylene, and the phenylene is optionally substituted by one R^{L}, and R^{L} is benzoxazolyl;
preferably, L¹ and L² are each independently selected from the group consisting of a direct bond,

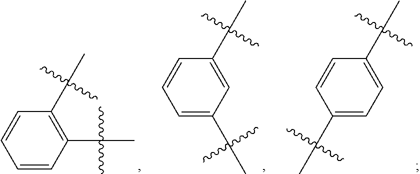 [SMILES: *c1ccccc1* ; *c1cccc(*)c1 ; *c1ccc(*)cc1]

R¹ and R² are each independently selected from the group consisting of diazaphenanthrolinyl, pyridinyl, pyrimidinyl, triazinyl, benzoxazolyl, benzothiazolyl, and the diazaphenanthrolinyl, pyridinyl, pyrimidinyl, triazinyl, benzoxazolyl and benzothiazolyl are each independently optionally substituted by one or two (preferably two) R^{R};
preferably, R¹ and R² are each independently selected from the group consisting of diazaphenanthrolinyl, pyridinyl, pyrimidinyl, triazinyl, and benzoxazolyl, and the pyridinyl, pyrimidinyl, triazinyl, benzoxazolyl are each independently optionally substituted by one or two (preferably two) R^{R};
more preferably, R¹ and R² are each independently selected from o-diazaphenanthrolinyl, pyridinyl, pyrimidinyl, 1,3,5-triazinyl, benzoxazolyl, and the pyridinyl, pyrimidinyl, 1,3,5-triazinyl, benzoxazolyl are each independently optionally substituted by one or two (preferably two) R^{R};
further preferably, R¹ and R² are each independently selected from the group consisting of

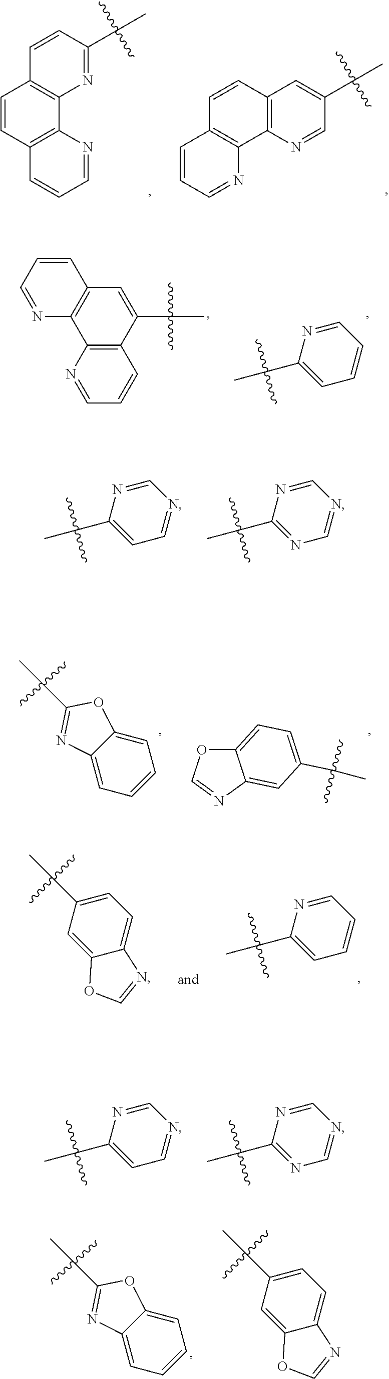

are each independently optionally substituted by one or two R^{R};
R^{R} is selected from the group consisting of phenyl, biphenyl, and fluorenyl, and the phenyl, biphenyl, and fluorenyl are each independently optionally substituted by two C₁-C₆ alkyl groups;
preferably, R^{R} is selected from the group consisting of phenyl, biphenyl, and fluorenyl, and the fluorenyl is optionally substituted by two C₁-C₆ alkyl groups;
more preferably, R^{R} is selected from the group consisting of phenyl, biphenyl, and fluorenyl, and the fluorenyl is optionally substituted by two methyl groups;
most preferably, R^{R} is selected from the group consisting of

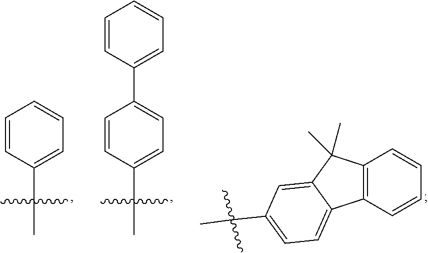

most preferably, R¹ and R² are each independently selected from the group consisting of:

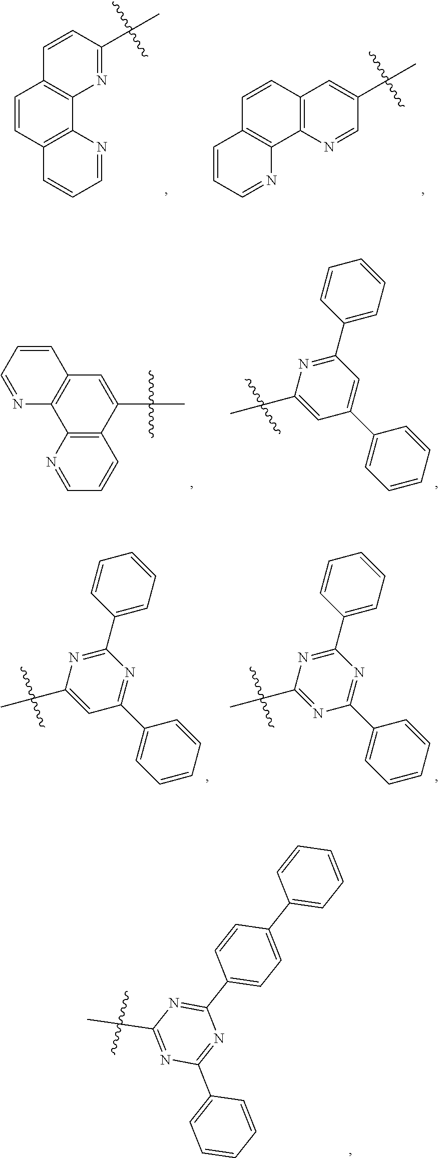

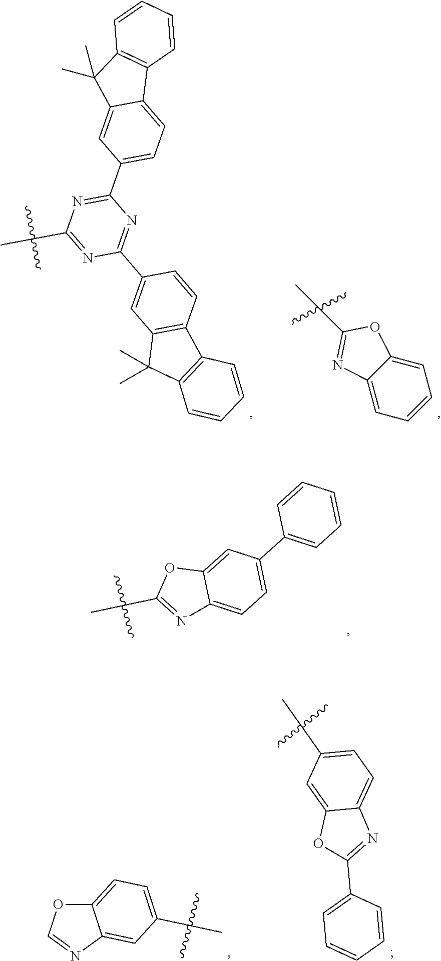 [SMILES: *c1nc(-c2ccc3c(c2)C(C)(C)c2ccccc2-3)nc(-c2ccc3c(c2)C(C)(C)c2ccccc2-3)n1 ; *c1nc2ccccc2o1 ; *c1nc2ccc(-c3ccccc3)cc2o1 ; *c1ccc2ocnc2c1 ; *c1ccc2nc(-c3ccccc3)oc2c1]

most preferably, R¹-L¹, R²-L²- are each independently selected from the group consisting of:

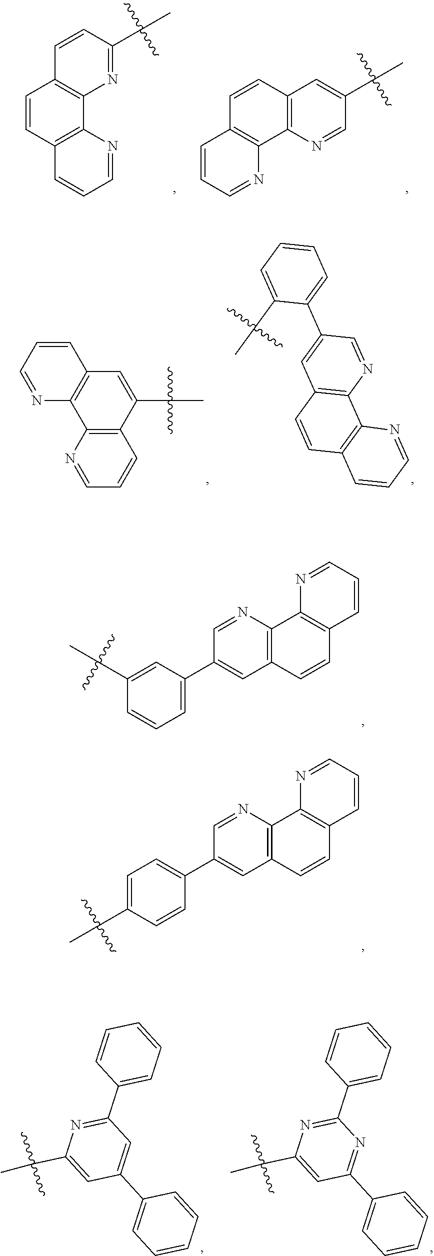

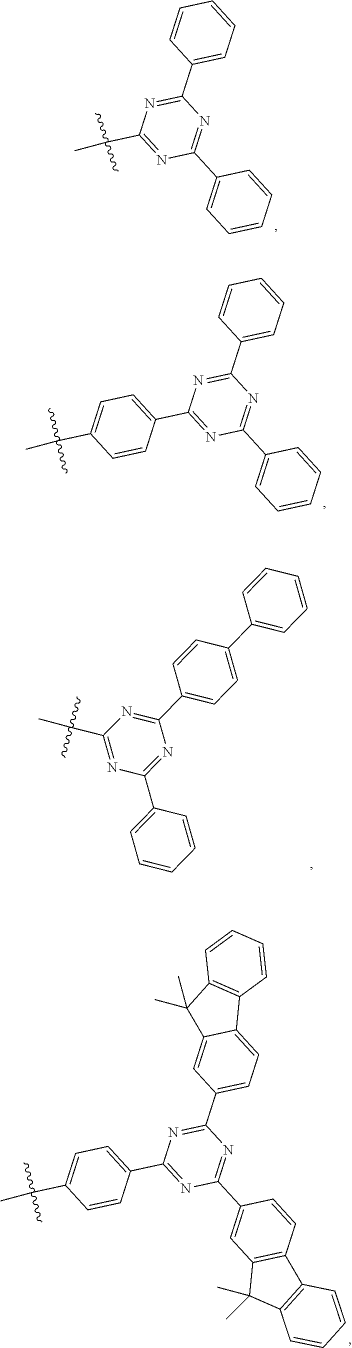 [SMILES: *c1nc(-c2ccccc2)nc(-c2ccccc2)n1 ; *c1ccc(-c2nc(-c3ccccc3)nc(-c3ccccc3)n2)cc1 ; *c1nc(-c2ccccc2)nc(-c2ccc(-c3ccccc3)cc2)n1 ; *c1ccc(-c2nc(-c3ccc4c(c3)C(C)(C)c3ccccc3-4)nc(-c3ccc4c(c3)C(C)(C)c3ccccc3-4)n2)cc1]

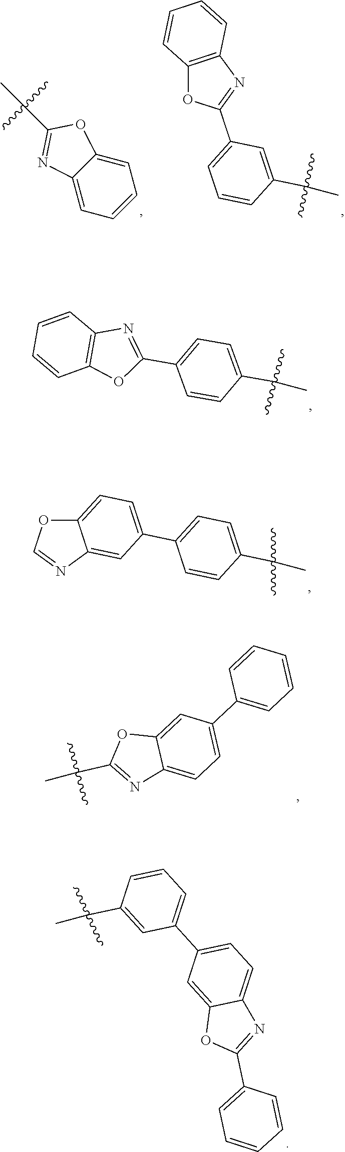 [SMILES: *c1nc2ccccc2o1 ; *c1cccc(-c2nc3ccccc3o2)c1 ; *c1ccc(-c2nc3ccccc3o2)cc1 ; *c1ccc(-c2ccc3ocnc3c2)cc1 ; *c1nc2ccc(-c3ccccc3)cc2o1 ; *c1cccc(-c2ccc3nc(-c4ccccc4)oc3c2)c1]

In the ninth aspect of the present disclosure, there is provided a compound represented by Formula II-3, or a stereoisomer or tautomer of the compound,

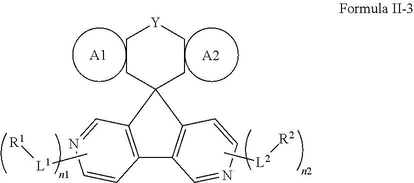

wherein:
Ring A1 and Ring A2 are each independently selected from benzene ring and naphthalene ring;
preferably, Ring A1 and Ring A2 are benzene rings;
Y is selected from —O—, —S—, —CR^{a}R^{b}—;
R^{a} and R^{b} are each independently selected from hydrogen and C₁-C₆ alkyl;
preferably, R^{a} and R^{b} are each independently selected from C₁-C₆ alkyl;
more preferably, R^{a} and R^{b} are methyl groups;
preferably, Y is selected from —O—, —S—,

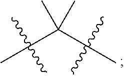 [SMILES: *C(*)(C)C]

more preferably, Y is selected from —O—, —S—;
n1 and n2 are each independently selected from 0 and 1, and n1 and n2 are not 0 at the same time;
preferably,

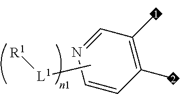

is selected from the group consisting of

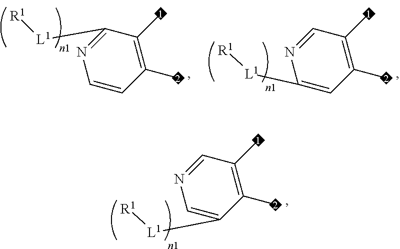

wherein, Carbon atom No. 1 is bonded to the central spiro carbon atom, and Carbon atom No. 2 is bonded to a carbon atom of the aromatic ring on the right;
preferably,

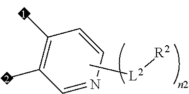

is selected from the group consisting of

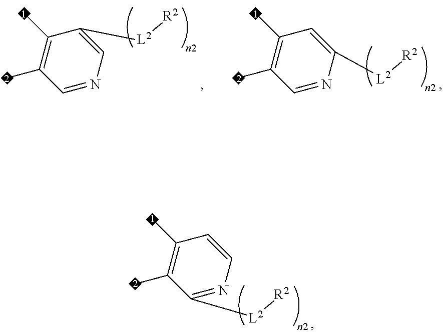

wherein, Carbon atom No. 1 is bonded to the central spiro carbon atom, and Carbon atom No. 2 is bonded to a carbon atom of the aromatic ring on the left;
L¹ and L² are each independently selected from the group consisting of a direct bond and phenylene, and the phenylene is optionally substituted by one R^{L}, and R^{L} is benzoxazolyl;
preferably, L¹ and L² are each independently selected from the group consisting of a direct bond

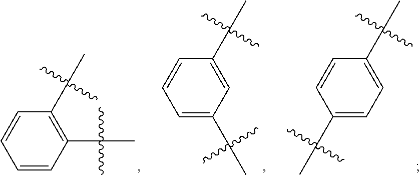 [SMILES: *c1ccccc1* ; *c1cccc(*)c1 ; *c1ccc(*)cc1]

R¹ and R² are each independently selected from the group consisting of diazaphenanthrolinyl, pyrimidinyl, triazinyl, benzoxazolyl, benzothiazolyl, and the diazaphenanthrolinyl, pyrimidinyl, triazinyl, benzoxazolyl and benzothiazolyl are each independently optionally substituted by one or two (preferably two) R^{R};
preferably, R¹ and R² are each independently selected from the group consisting of diazaphenanthrolinyl, pyrimidinyl, triazinyl, and benzoxazolyl, and the pyrimidinyl, triazinyl, and benzoxazolyl are each independently optionally substituted by one or two (preferably two) R^{R};
more preferably, R¹ and R² are each independently selected from o-diazaphenanthrolinyl, pyrimidinyl, 1,3,5-triazinyl, and benzoxazolyl, and the pyrimidinyl, 1,3,5-triazinyl, and benzoxazolyl are each independently optionally substituted by one or two (preferably two) R^{R};
further preferably, R¹ and R² are each independently selected from the group consisting of

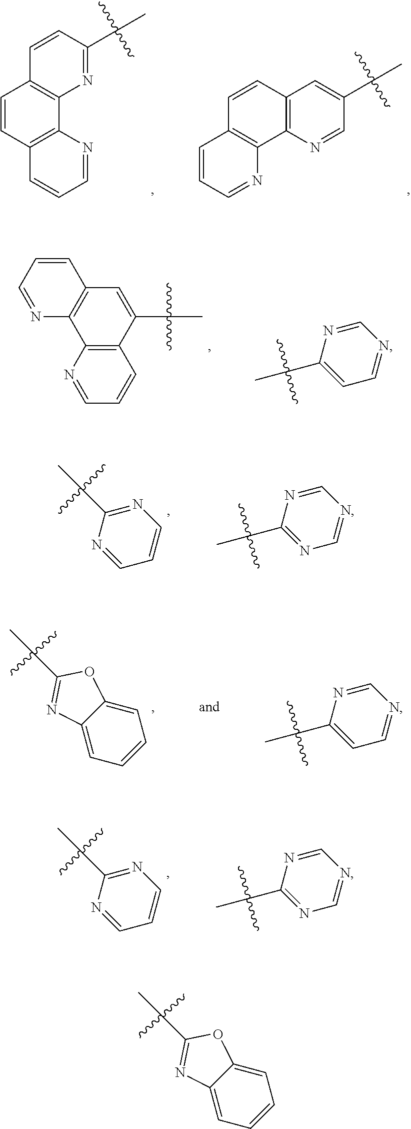

are each independently optionally substituted by one or two R^{R};
R^{R} is selected from phenyl and fluorenyl, and the phenyl and fluorenyl are each independently optionally substituted by two C₁-C₆ alkyl groups;
preferably, R^{R} is selected from phenyl and fluorenyl, and the fluorenyl is optionally substituted by two C₁-C₆ alkyl groups;
more preferably, R^{R} is selected from phenyl and fluorenyl, and the fluorenyl is optionally substituted by two methyl groups;
most preferably, R^{R} is selected from

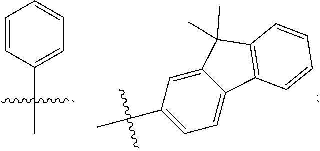 [SMILES: *c1ccccc1 ; *c1ccc2c(c1)C(C)(C)c1ccccc1-2]

most preferably, R¹ and R² are each independently selected from the group consisting of:

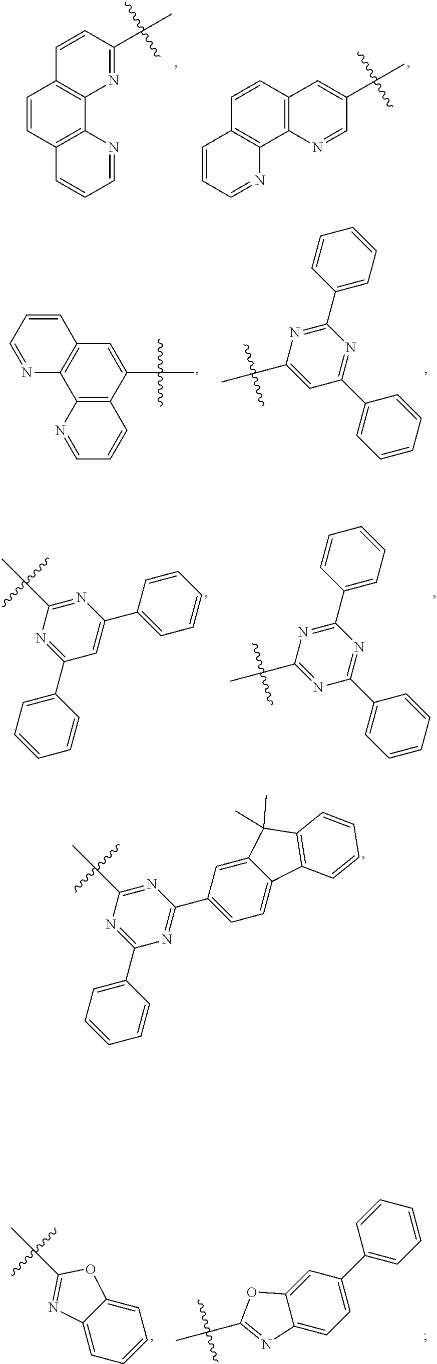

most preferably, R¹-L¹-, R²-L²- are each independently selected from the group consisting of:

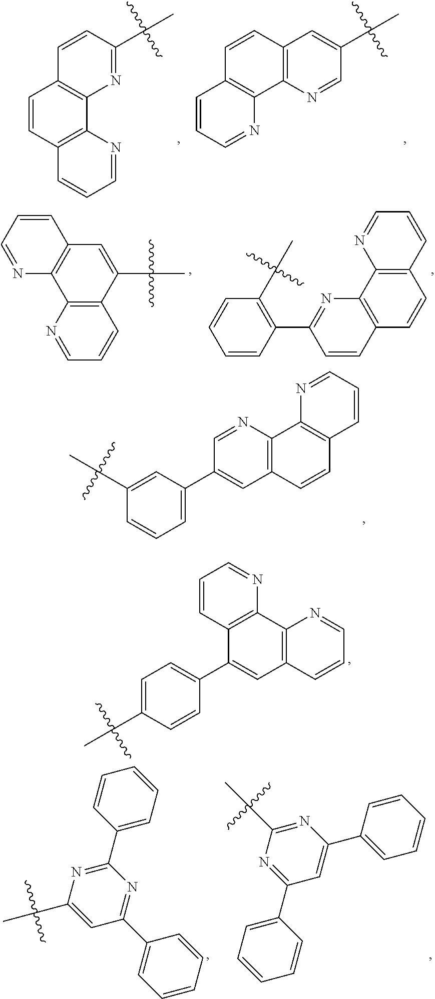

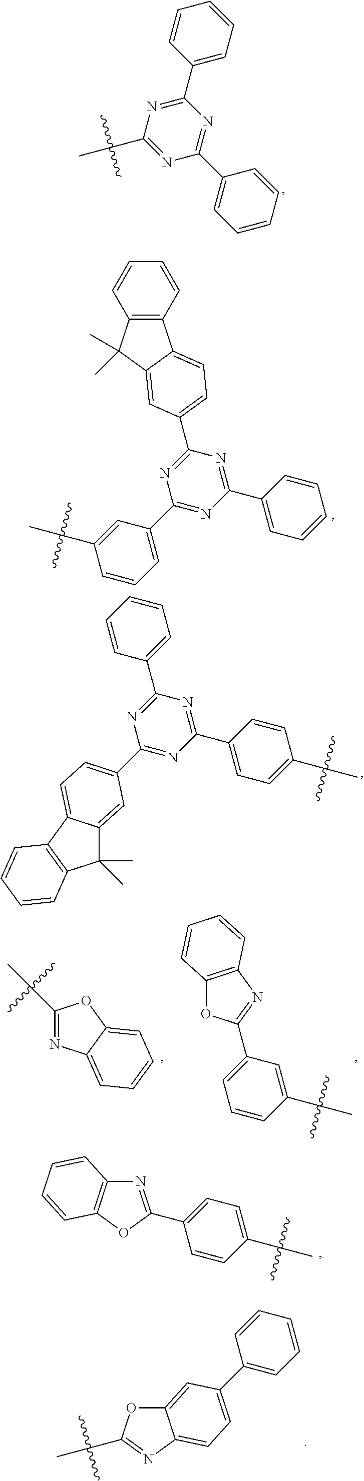

In the tenth aspect of the present disclosure, there is provided a compound represented by Formula II-4, or a stereoisomer or tautomer of the compound,

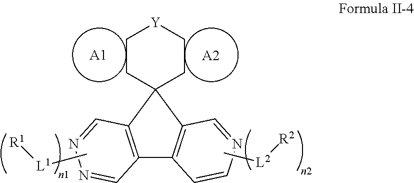

wherein:
Ring A1 and Ring A2 are each independently selected from benzene ring and naphthalene ring;
preferably, Ring A1 and Ring A2 are benzene rings;
Y is selected from —O—, —S—, —CR^{a}R^{b}—;
R^{a} and R^{b} are each independently selected from hydrogen and C₁-C₆ alkyl;
preferably, R^{a} and R^{b} are each independently selected from C₁-C₆ alkyl;
more preferably, R^{a} and R^{b} are methyl groups;
preferably, Y is selected from —O—, —S—,

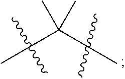 [SMILES: *C(*)(C)C]

more preferably, Y is selected from —O—, —S—;
n1 and n2 are each independently selected from 0 and 1, and n1 and n2 are not 0 at the same time;
preferably,

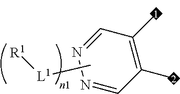

is selected from

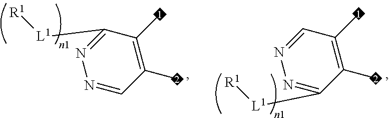

wherein Carbon atom No. 1 is bonded to the central spiro carbon atom, and Carbon atom No. 2 is bonded to a carbon atom of the aromatic ring on the right;
preferably,

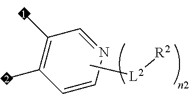

is selected from the group consisting of

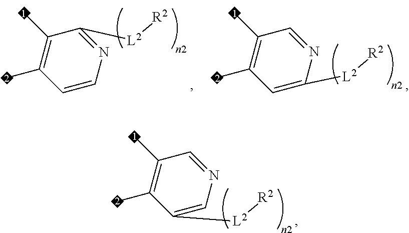

wherein, Carbon atom No. 1 is bonded to the central spiro carbon atom, and Carbon atom No. 2 is bonded to a carbon atom of the aromatic ring on the left;
L¹ and L² are each independently selected from the group consisting of a direct bond, phenylene, and naphthylene, and the phenylene and naphthylene are each independently optionally substituted by one R^{L};
preferably, L¹ and L² are each independently selected from the group consisting of a direct bond and phenylene, and the phenylene is optionally substituted by one R^{L};
more preferably, L¹ and L² are each independently selected from the group consisting of a direct bond,

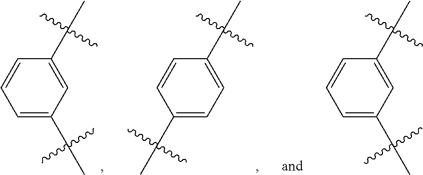 [SMILES: *c1cccc(*)c1 ; *c1ccc(*)cc1 ; *c1cccc(*)c1]

is optionally substituted by one R^{L};
R^{L} is selected from benzoxazolyl and benzothiazolyl;
preferably, R^{L} is benzoxazolyl;
more preferably, R^{L} is

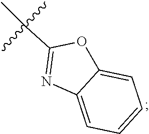 [SMILES: *c1nc2ccccc2o1]

most preferably, L¹ and L² are each independently selected from the group consisting of a direct bond,

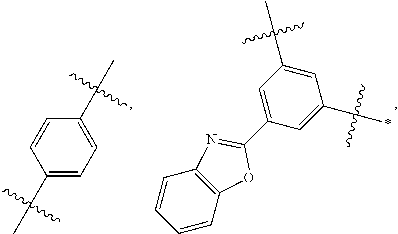

where the bond with marker * is bonded to the parent ring, and the bond without marker * is bonded to R¹ or R²;
R¹ and R² are each independently selected from the group consisting of diazaphenanthrolinyl, pyridinyl, triazinyl, benzoxazolyl, and benzothiazolyl, and the diazaphenanthrolinyl, pyridinyl, triazinyl, benzoxazolyl and benzothiazolyl are each independently optionally substituted by one or two (preferably two) R^{R};
preferably, R¹ and R² are each independently selected from the group consisting of diazaphenanthrolinyl, pyridinyl, triazinyl, and benzoxazolyl, and the pyridinyl, triazinyl, and benzoxazolyl are each independently optionally substituted by one or two (preferably two) R^{R};
more preferably, R¹ and R² are each independently selected from o-diazaphenanthrolinyl, pyridinyl, 1,3,5-triazinyl, and benzoxazolyl, and the pyridinyl, 1,3,5-triazinyl, and benzoxazolyl are each independently optionally substituted by one or two (preferably two) R^{R};
further preferably, R¹ and R² are each independently selected from the group consisting of

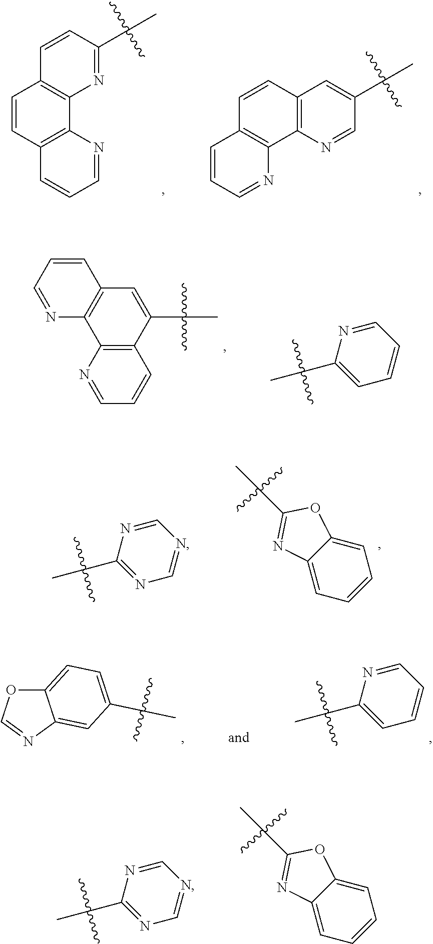

are each independently optionally substituted by one or two R^{R};
R^{R} is selected from phenyl and biphenyl, and the phenyl and biphenyl are independently optionally substituted by two C₁-C₆ alkyl groups;
preferably, R^{R} is selected from phenyl and biphenyl;
more preferably, R^{R} is selected from

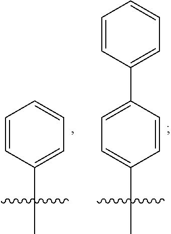

most preferably, R¹ and R² are each independently selected from the group consisting of:

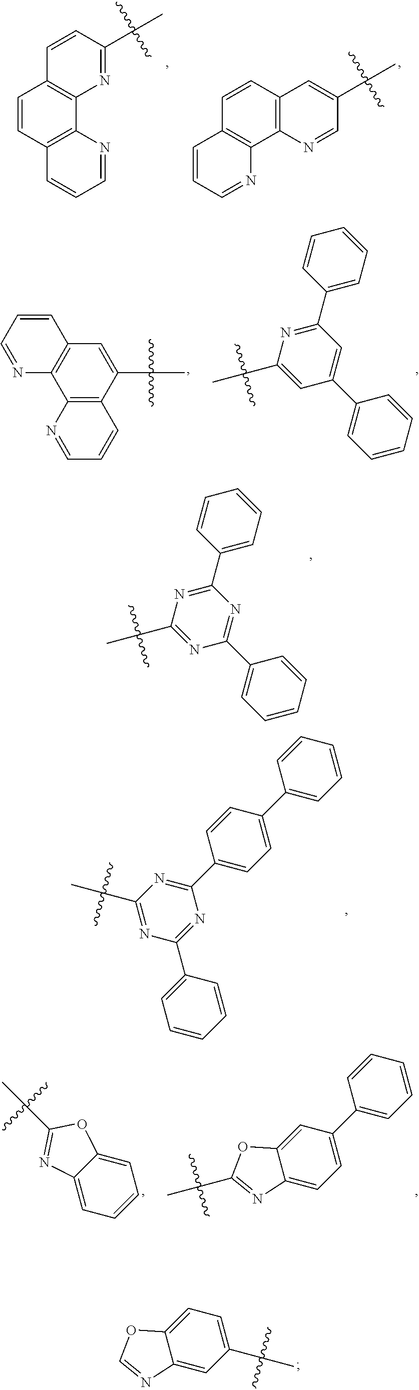

most preferably, R¹-L¹-, R²-L²- are each independently selected from the group consisting of:

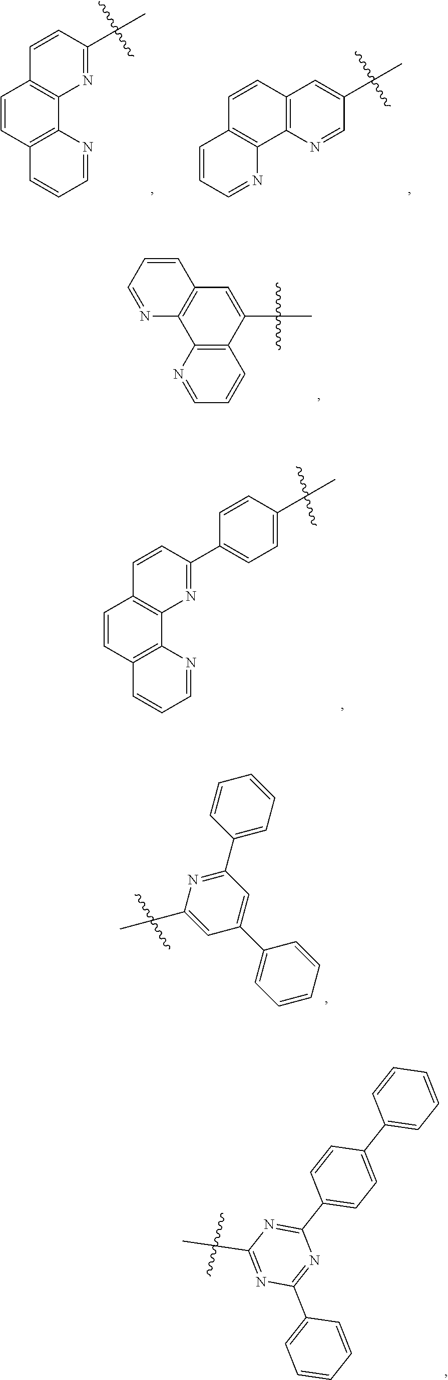 [SMILES: *c1ccc2ccc3cccnc3c2n1 ; *c1cnc2c(ccc3cccnc32)c1 ; *c1cc2cccnc2c2ncccc12 ; *c1ccc(-c2ccc3ccc4cccnc4c3n2)cc1 ; *c1cc(-c2ccccc2)cc(-c2ccccc2)n1 ; *c1nc(-c2ccccc2)nc(-c2ccc(-c3ccccc3)cc2)n1]

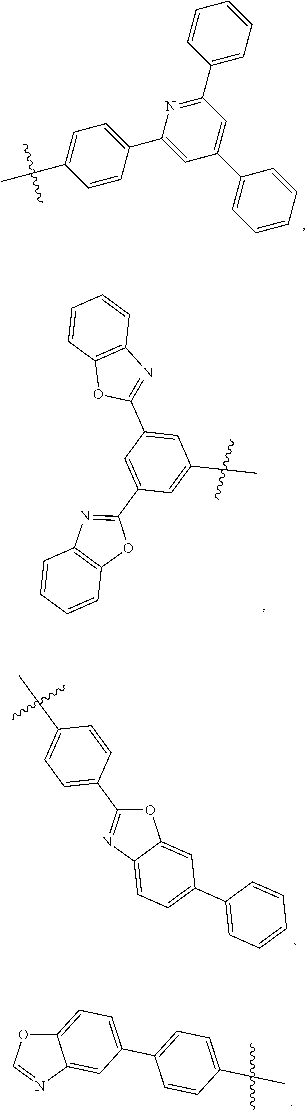 [SMILES: *c1ccc(-c2cc(-c3ccccc3)cc(-c3ccccc3)n2)cc1 ; *c1cc(-c2nc3ccccc3o2)cc(-c2nc3ccccc3o2)c1 ; *c1ccc(-c2nc3ccc(-c4ccccc4)cc3o2)cc1 ; *c1ccc(-c2ccc3ocnc3c2)cc1]

In the eleventh aspect of the present disclosure, there is provided a compound represented by Formula II-5, or a stereoisomer or tautomer of the compound,

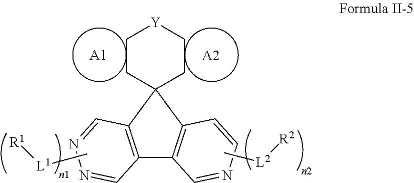

wherein:
Ring A1 and Ring A2 are each independently selected from benzene ring and naphthalene ring;
preferably, Ring A1 and Ring A2 are benzene rings;
Y is selected from —O—, —S—, —CR^{a}R^{b}—;
R^{a} and R^{b} are each independently selected from hydrogen and C₁-C₆ alkyl;
preferably, R^{a} and R^{b} are each independently selected from C₁-C₆ alkyl;
more preferably, R^{a} and R^{b} are methyl groups;
preferably, Y is selected from —O—, —S—,

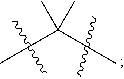 [SMILES: *C(*)(C)C]

more preferably, Y is selected from —O—, —S—;
n1 and n2 are each independently selected from 0 and 1, and n1 and n2 are not 0 at the same time;
preferably,

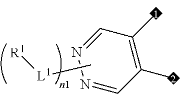

is selected from

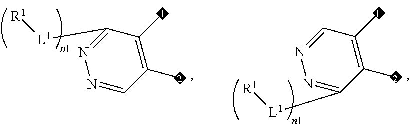

wherein Carbon atom No. 1 is bonded to the central spiro carbon atom, and Carbon atom No. 2 is bonded to a carbon atom of the aromatic ring on the right;
preferably,

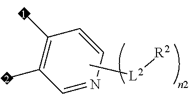

is selected from the group consisting of

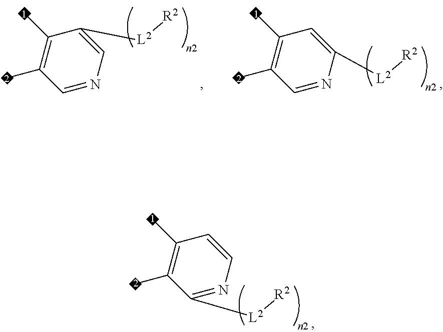

wherein, Carbon atom No. 1 is bonded to the central spiro carbon atom, and Carbon atom No. 2 is bonded to a carbon atom of the aromatic ring on the left;
L¹ and L² are each independently selected from the group consisting of a direct bond, phenylene, and naphthylene, and the phenylene and naphthylene are each independently optionally substituted by one R^{L};
preferably, L¹ and L² are each independently selected from the group consisting of a direct bond and phenylene, and the phenylene is optionally substituted by one R^{L};
more preferably, L¹ and L² are each independently selected from the group consisting of a direct bond,

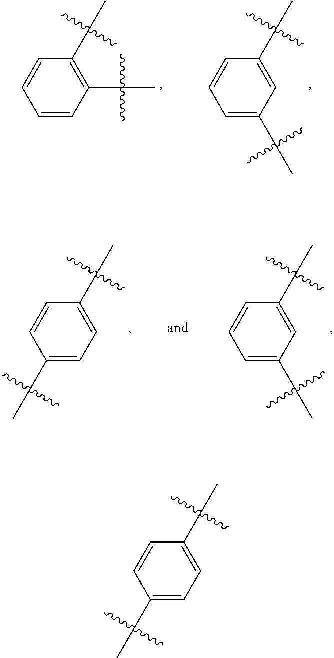 [SMILES: *c1ccccc1* ; *c1cccc(*)c1 ; *c1ccc(*)cc1 ; *c1cccc(*)c1 ; *c1ccc(*)cc1]

are each independently optionally substituted by one R^{L};
R^{L} is selected from benzoxazolyl and benzothiazolyl;
preferably, R^{L} is benzoxazolyl;
more preferably, R^{L} is selected from

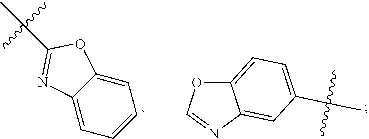 [SMILES: *c1nc2ccccc2o1 ; *c1ccc2ocnc2c1]

most preferably, L¹ and L² are each independently selected from the group consisting of:
a direct bond,

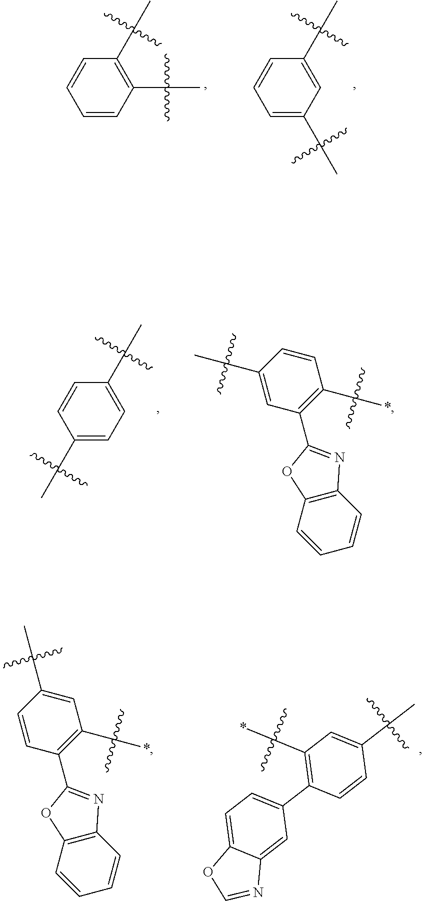

wherein, the bond with marker * is bonded to the parent ring, and the bond without marker * is bonded to R¹ or R²;
R¹ and R² are each independently selected from the group consisting of diazaphenanthrolinyl, pyrimidinyl, triazinyl, benzoxazolyl, benzothiazolyl, and the diazaphenanthrolinyl, pyrimidinyl, triazinyl, benzoxazolyl and benzothiazolyl are each independently optionally substituted by one or two (preferably two) R^{R};
preferably, R¹ and R² are each independently selected from the group consisting of diazaphenanthrolinyl, pyrimidinyl, triazinyl, and benzoxazolyl, and the pyrimidinyl, triazinyl, and benzoxazolyl are each independently optionally substituted by one or two (preferably two) R^{R};
more preferably, R¹ and R² are each independently selected from o-diazaphenanthrolinyl, pyrimidinyl, 1,3,5-triazinyl, and benzoxazolyl, and the pyrimidinyl, 1,3,5-triazinyl, and benzoxazolyl are each independently optionally substituted by one or two (preferably two) R^{R};
further preferably, R¹ and R² are each independently selected from the group consisting of

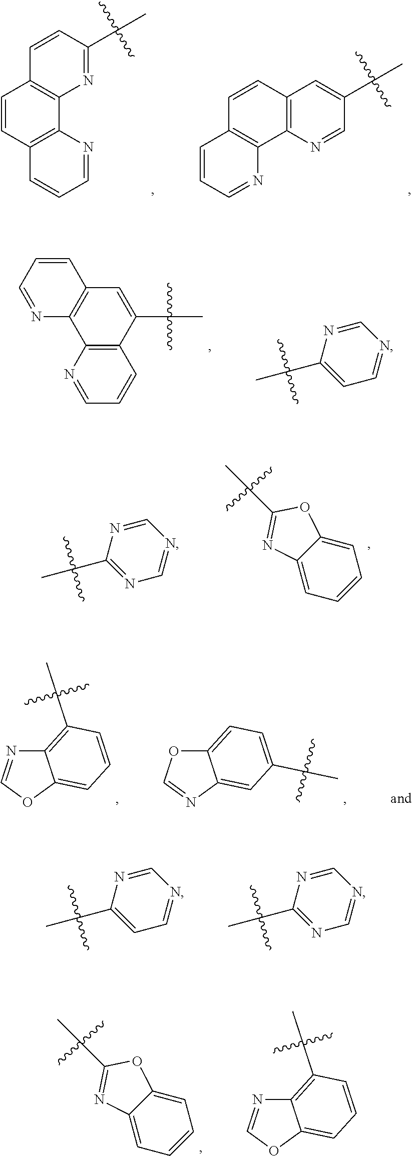

are each independently optionally substituted by one or two R^{R};
R^{R} is selected from the group consisting of phenyl, biphenyl, and naphthyl, and the phenyl, biphenyl, and naphthyl are each independently optionally substituted by two C₁-C₆ alkyl groups;
preferably, R^{R} is selected from the group consisting of phenyl, biphenyl, and naphthyl;
more preferably, R^{R} is selected from the group consisting of

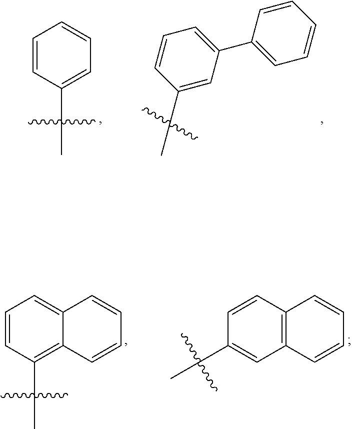 [SMILES: *c1ccccc1 ; *c1cccc(-c2ccccc2)c1 ; *c1cccc2ccccc12 ; *c1ccc2ccccc2c1]

most preferably, R¹ and R² are each independently selected from the group consisting of:

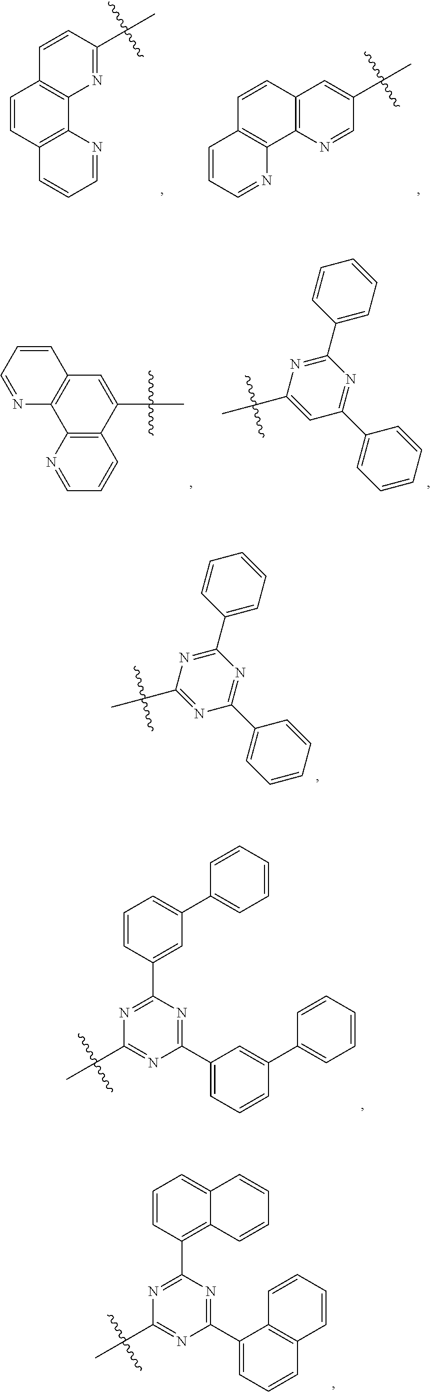

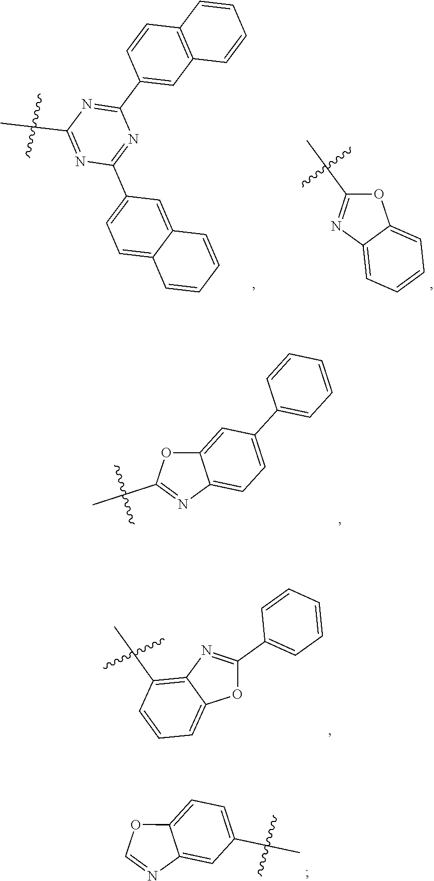 [SMILES: *c1nc(-c2ccc3ccccc3c2)nc(-c2ccc3ccccc3c2)n1 ; *c1nc2ccccc2o1 ; *c1nc2ccc(-c3ccccc3)cc2o1 ; *c1cccc2oc(-c3ccccc3)nc12 ; *c1ccc2ocnc2c1]

most preferably, R¹-L¹-, R²-L²- are each independently selected from the group consisting of:

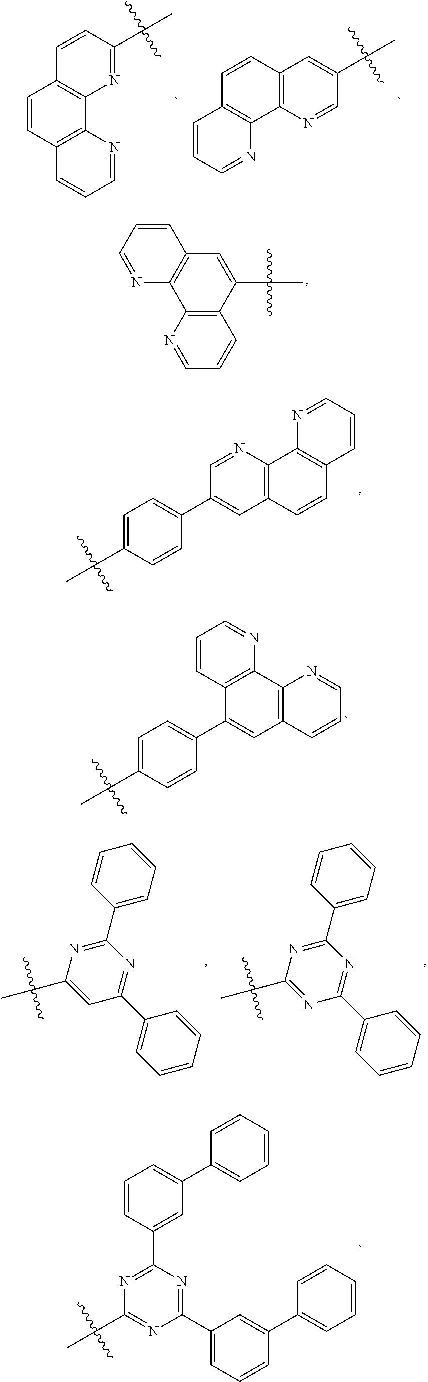

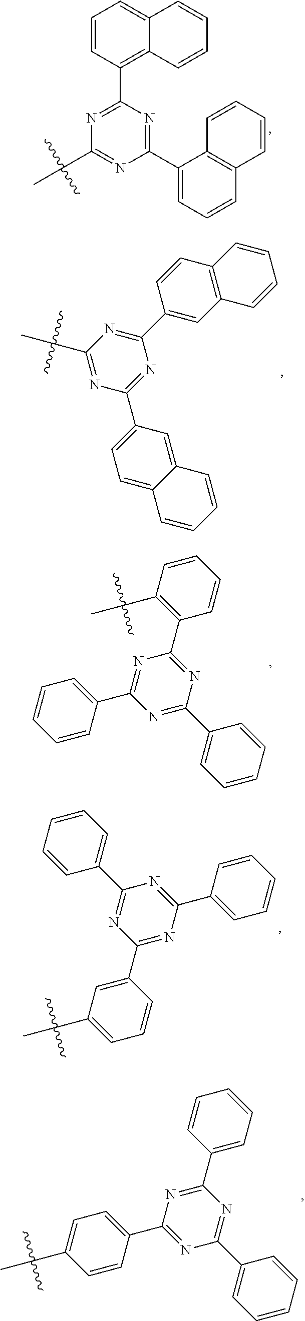

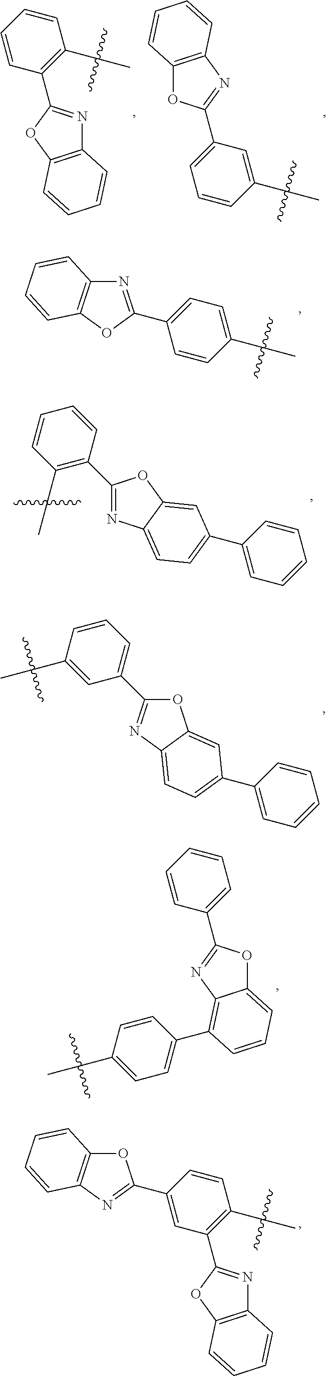

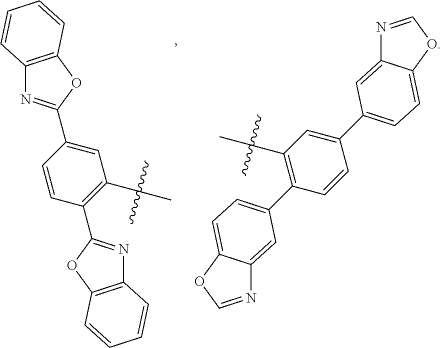 [SMILES: *c1cc(-c2nc3ccccc3o2)ccc1-c1nc2ccccc2o1 ; *c1cc(-c2ccc3ocnc3c2)ccc1-c1ccc2ocnc2c1]

In the twelfth aspect of the present disclosure, there is provided a compound represented by Formula Oc-1, Formula Oc-2 or Formula Oc-3, or a stereoisomer or tautomer of the compound,

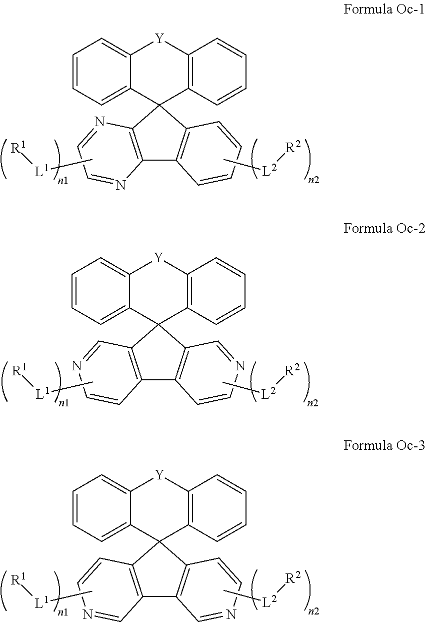

wherein:
Y is selected from O and S;
n1 and n2 are each independently selected from 0 and 1, and n1 and n2 are not 0 at the same time;
L¹ and L² are each independently selected from the group consisting of a direct bond, phenylene, naphthylene, and biphenylene, and the phenylene, naphthylene, and biphenylene are each independently optionally substituted by one R^{L};
preferably, L¹ and L² are each independently selected from the group consisting of a direct bond, phenylene, naphthylene, and biphenylene, and the phenylene is optionally substituted by one R^{L};
more preferably, L¹ and L² are each independently selected from the group consisting of a direct bond,

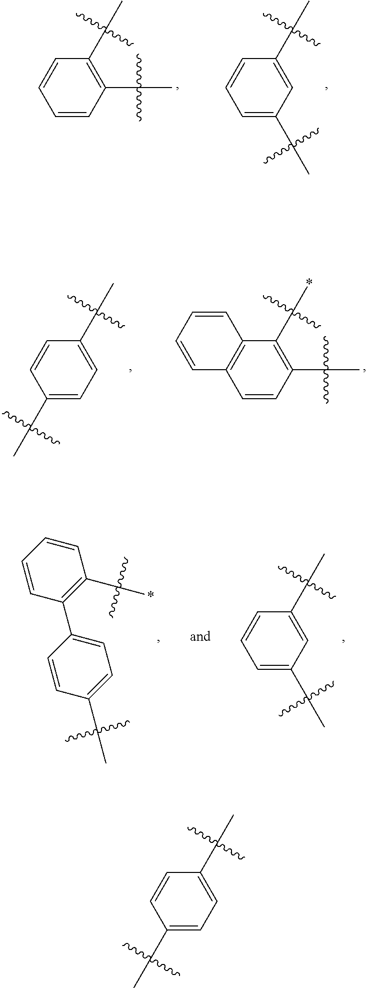

are each independently optionally substituted by one R^{L}, wherein the bond with marker * is bonded to the parent ring, and the bond without marker * is bonded to R¹ or R².
R^{L} is selected from benzoxazolyl and benzothiazolyl;
preferably, R^{L} is benzoxazolyl;
more preferably, R^{L} is selected from

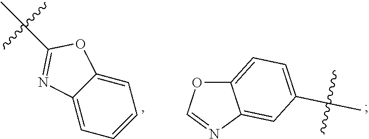 [SMILES: *c1nc2ccccc2o1 ; *c1ccc2ocnc2c1]

most preferably, L¹ and L² are each independently selected from the group consisting of:
a direct bond,

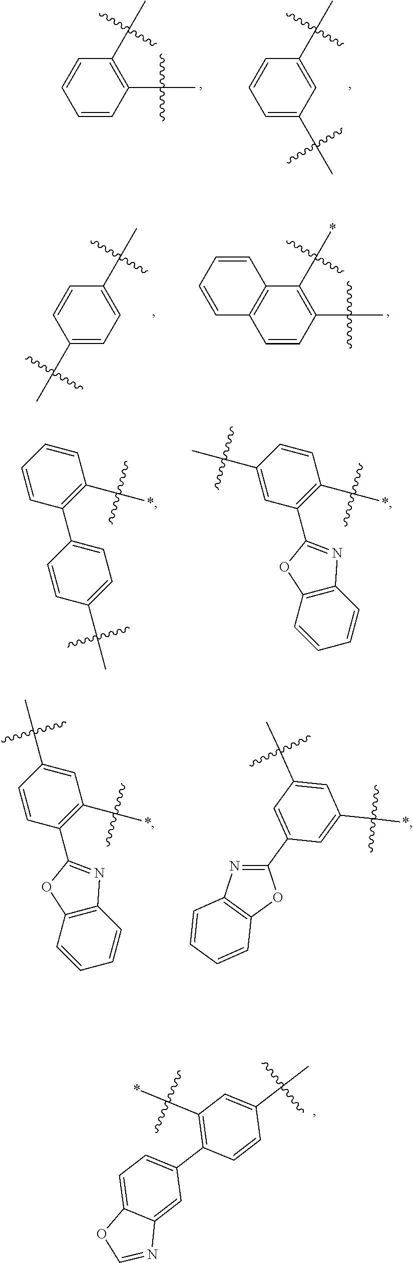

wherein, the bond with marker * is bonded to the parent ring, and the bond without marker * is bonded to R¹ or R²;
R¹ and R² are each independently selected from the group consisting of diazaphenanthrolinyl, pyridinyl, pyrimidinyl, triazinyl, benzoxazolyl, benzothiazolyl, and the diazaphenanthrolinyl, pyridinyl, pyrimidinyl, triazinyl, benzoxazolyl and benzothiazolyl are each independently optionally substituted by one or two R^{R};
preferably, R¹ and R² are each independently selected from the group consisting of diazaphenanthrolinyl, pyridinyl, pyrimidinyl, triazinyl, benzoxazolyl, benzothiazolyl, and the pyridinyl, pyrimidinyl, triazinyl, benzoxazolyl are each independently optionally substituted by one or two R^{R};
more preferably, R¹ and R² are each independently selected from o-diazaphenanthrolinyl, pyridinyl, pyrimidinyl, 1,3,5-triazinyl, benzoxazolyl, and the pyridinyl, pyrimidinyl, 1,3,5-triazinyl, benzoxazolyl are each independently optionally substituted by one or two R^{R};
further preferably, R¹ and R² are each independently selected from the group consisting of

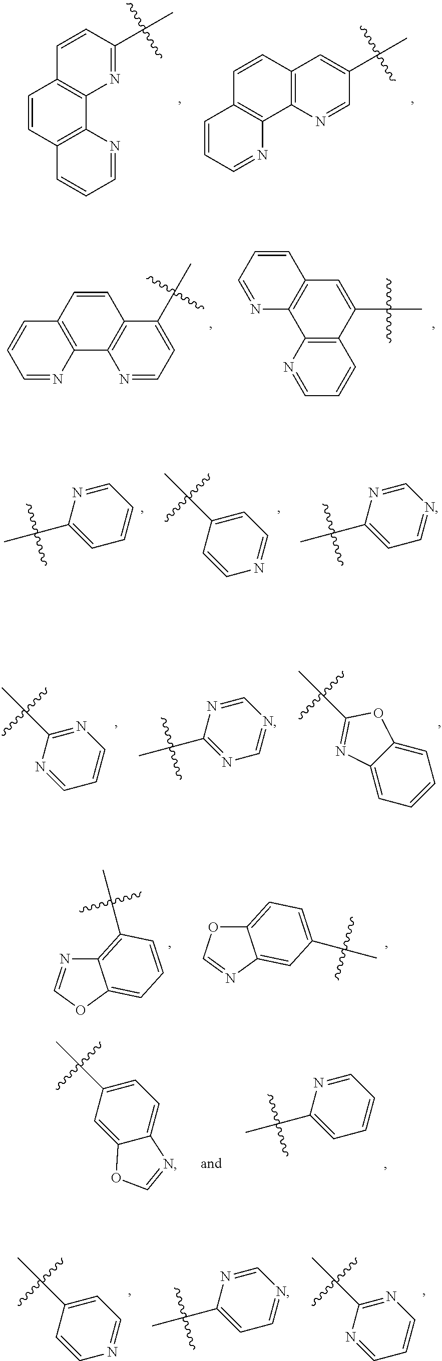

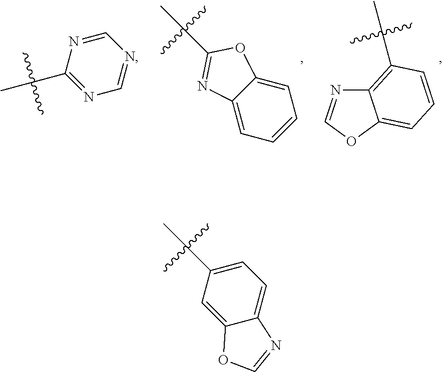

are each independently optionally substituted by one or two R^{R};
R^{R} is selected from the group consisting of phenyl, biphenyl, naphthyl, and fluorenyl, and the phenyl, biphenyl, naphthyl, and fluorenyl are each independently optionally substituted by two C₁-C₆ alkyl groups;
preferably, R^{R} is selected from the group consisting of phenyl, biphenyl, naphthyl, and fluorenyl, and the fluorenyl is optionally substituted by two C₁-C₆ alkyl groups;
more preferably, R^{R} is selected from the group consisting of phenyl, biphenyl, naphthyl, and fluorenyl, and the fluorenyl is optionally substituted by two methyl groups;
most preferably, R^{R} is selected from the group consisting of

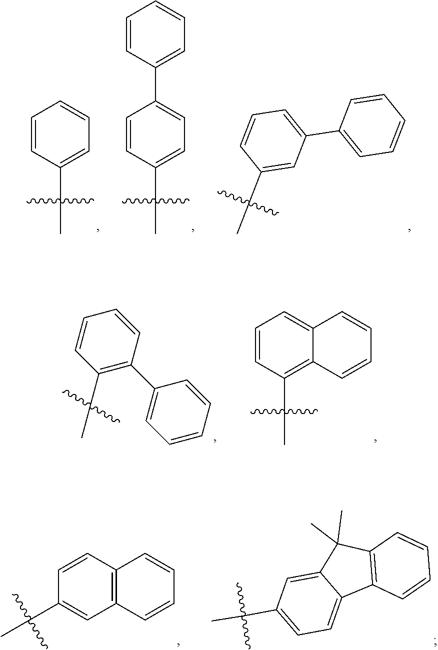

most preferably, R¹ and R² are each independently selected from the group consisting of:

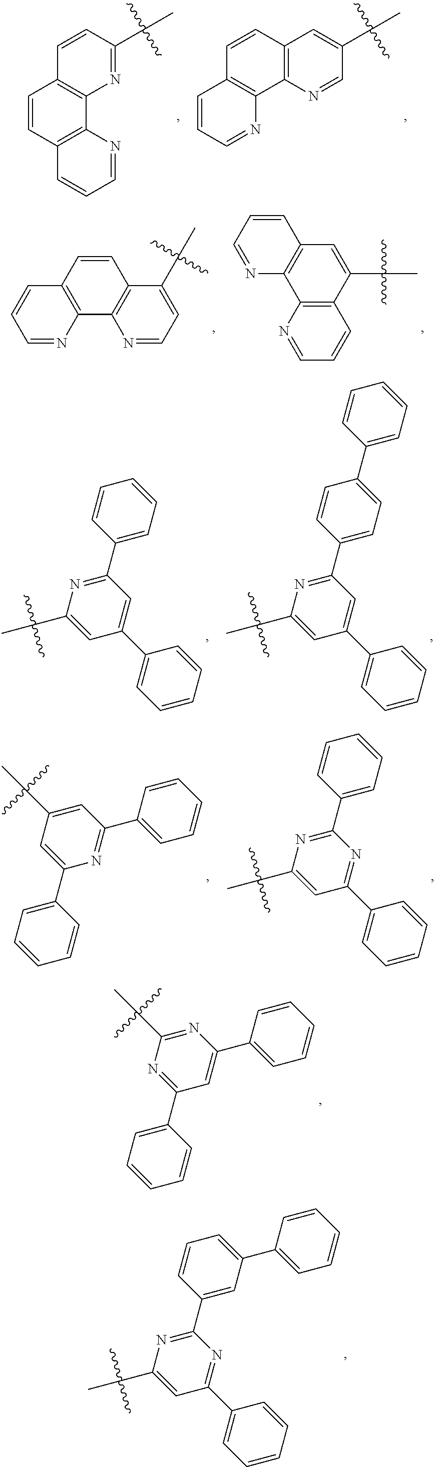

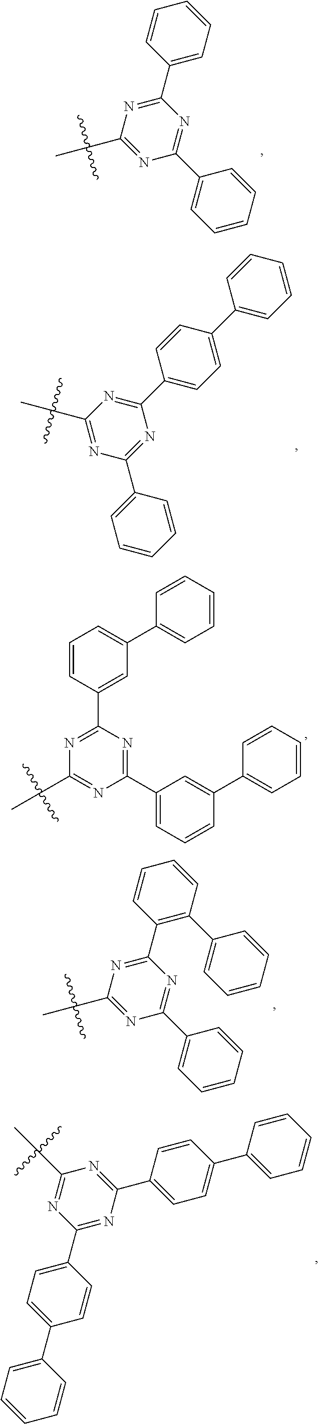

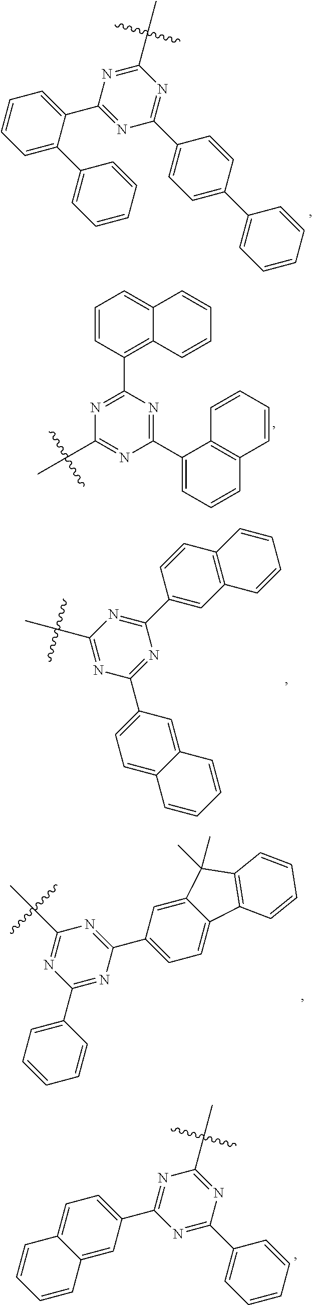

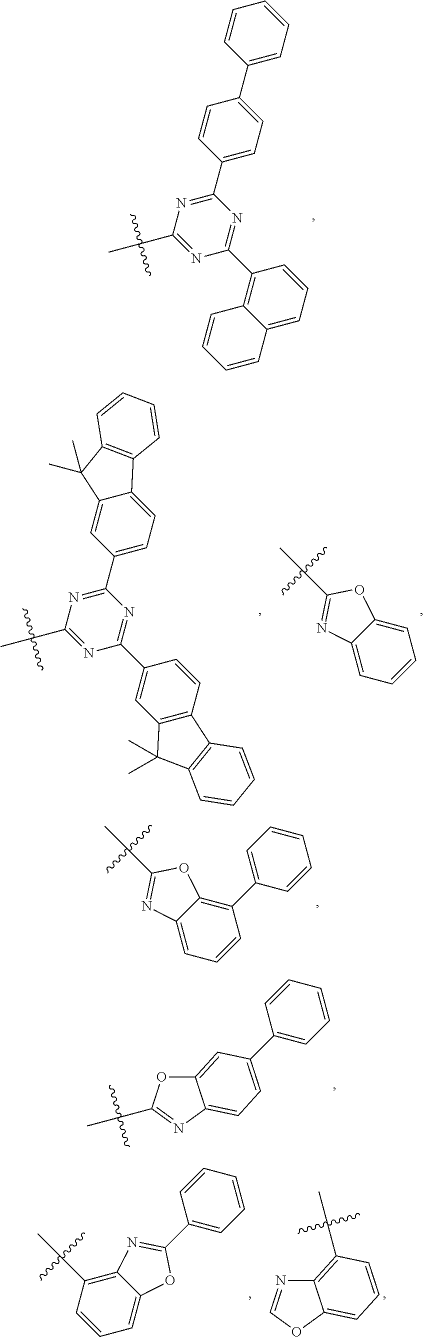

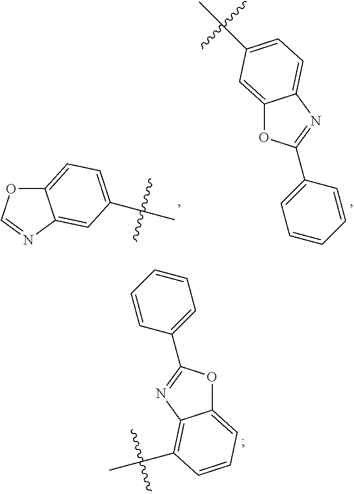 [SMILES: *c1ccc2ocnc2c1 ; *c1ccc2nc(-c3ccccc3)oc2c1 ; *c1cccc2oc(-c3ccccc3)nc12]

most preferably, R¹-L¹-, R²-L²- are each independently selected from the group consisting of:

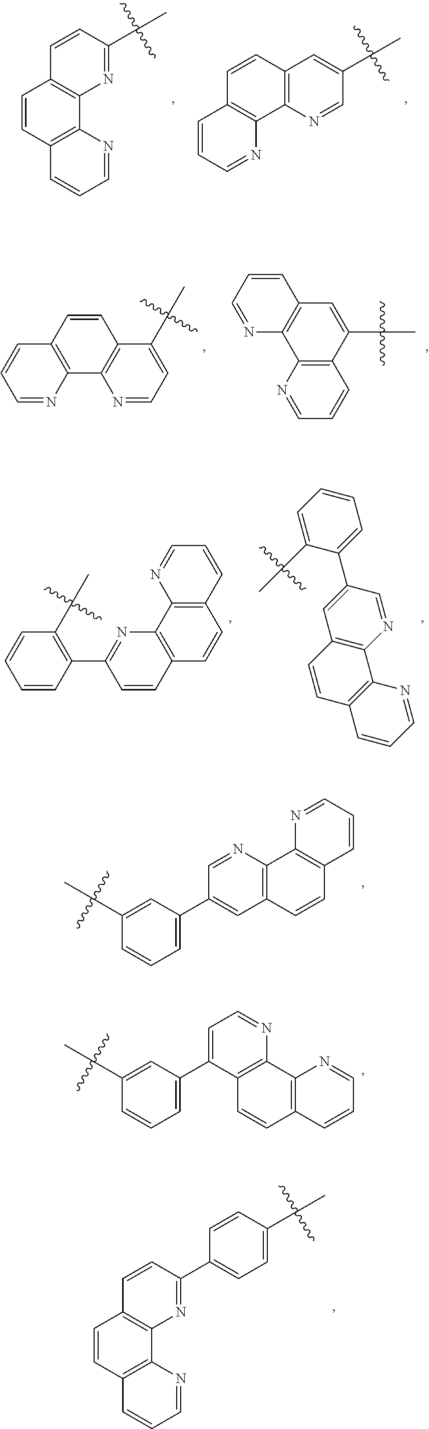

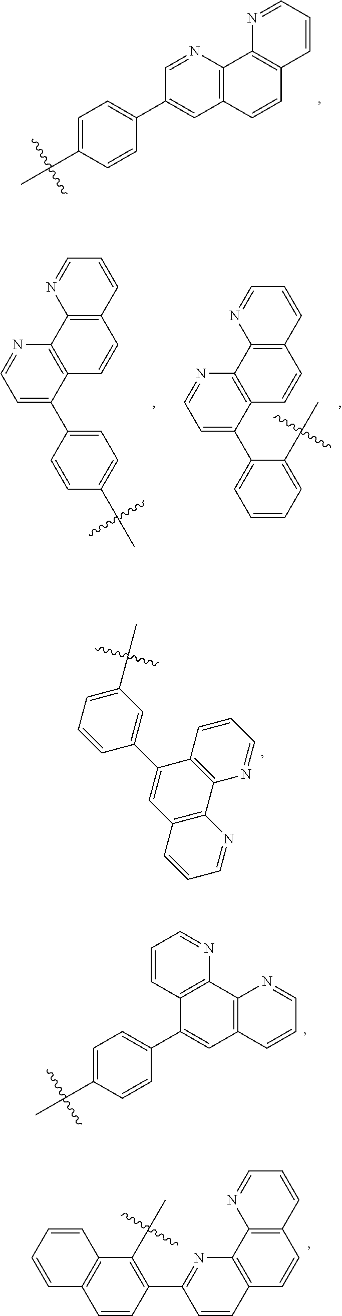 [SMILES: *c1ccc(-c2cnc3c(ccc4cccnc43)c2)cc1 ; *c1ccc(-c2ccnc3c2ccc2cccnc23)cc1 ; *c1ccccc1-c1ccnc2c1ccc1cccnc12 ; *c1cccc(-c2cc3cccnc3c3ncccc23)c1 ; *c1ccc(-c2cc3cccnc3c3ncccc23)cc1 ; *c1c(-c2ccc3ccc4cccnc4c3n2)ccc2ccccc12]

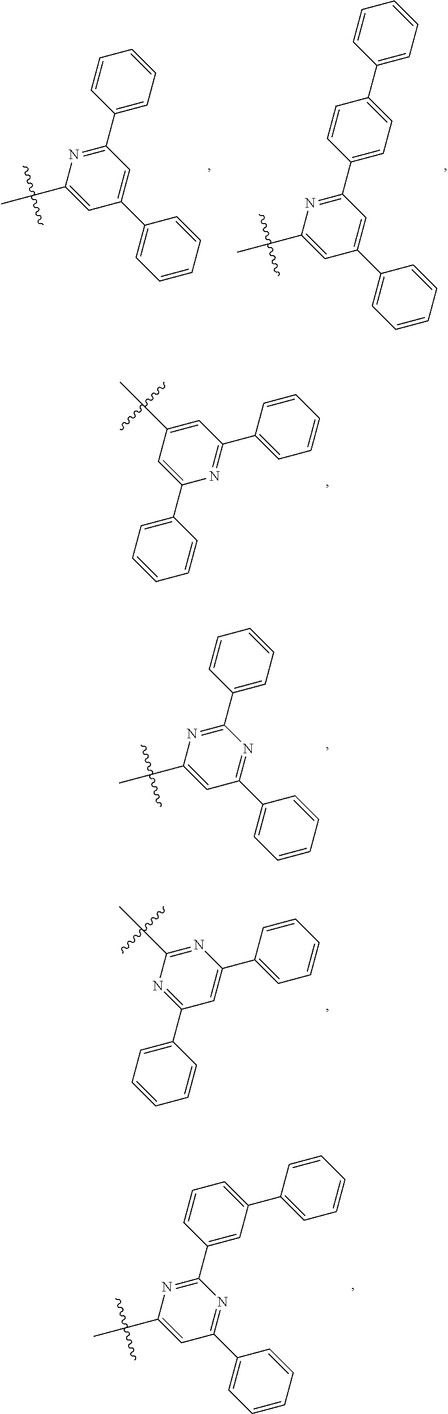 [SMILES: *c1cc(-c2ccccc2)cc(-c2ccccc2)n1 ; *c1cc(-c2ccccc2)cc(-c2ccc(-c3ccccc3)cc2)n1 ; *c1cc(-c2ccccc2)nc(-c2ccccc2)c1 ; *c1cc(-c2ccccc2)nc(-c2ccccc2)n1 ; *c1nc(-c2ccccc2)cc(-c2ccccc2)n1 ; *c1cc(-c2ccccc2)nc(-c2cccc(-c3ccccc3)c2)n1]

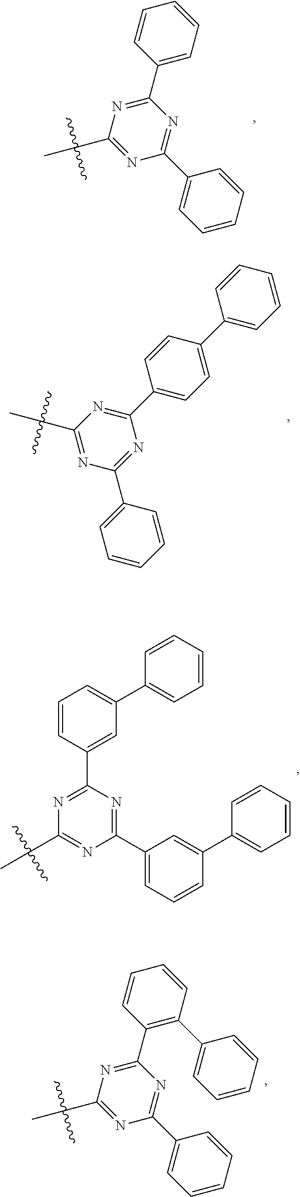

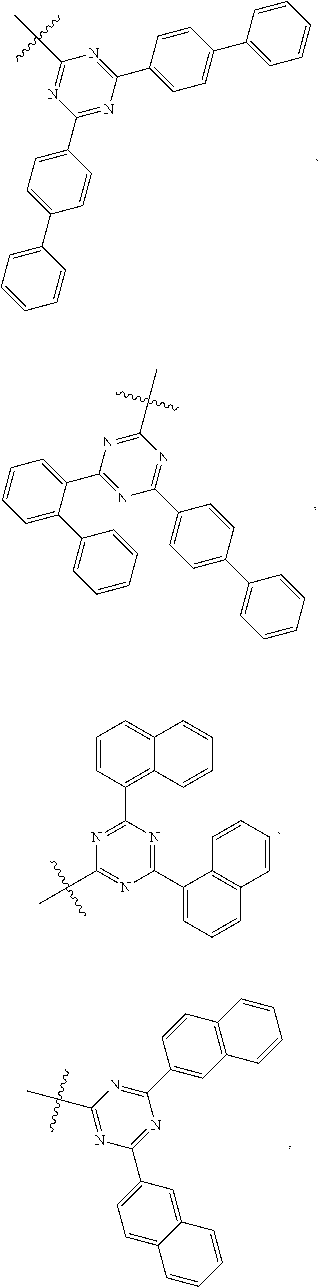 [SMILES: *c1nc(-c2ccc(-c3ccccc3)cc2)nc(-c2ccc(-c3ccccc3)cc2)n1 ; *c1nc(-c2ccc(-c3ccccc3)cc2)nc(-c2ccccc2-c2ccccc2)n1 ; *c1nc(-c2cccc3ccccc23)nc(-c2cccc3ccccc23)n1 ; *c1nc(-c2ccc3ccccc3c2)nc(-c2ccc3ccccc3c2)n1]

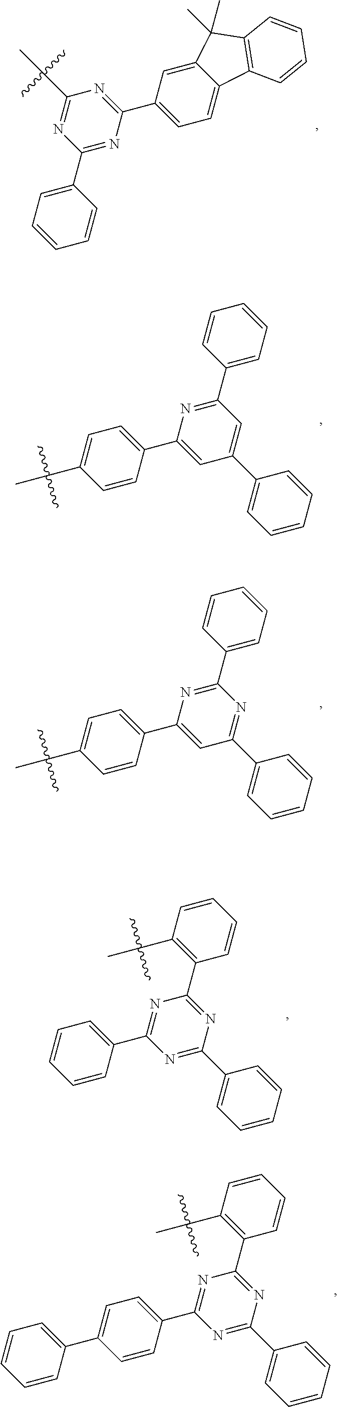 [SMILES: *c1nc(-c2ccccc2)nc(-c2ccc3c(c2)C(C)(C)c2ccccc2-3)n1 ; *c1ccc(-c2cc(-c3ccccc3)cc(-c3ccccc3)n2)cc1 ; *c1ccc(-c2cc(-c3ccccc3)nc(-c3ccccc3)n2)cc1 ; *c1ccccc1-c1nc(-c2ccccc2)nc(-c2ccccc2)n1 ; *c1ccccc1-c1nc(-c2ccccc2)nc(-c2ccc(-c3ccccc3)cc2)n1]

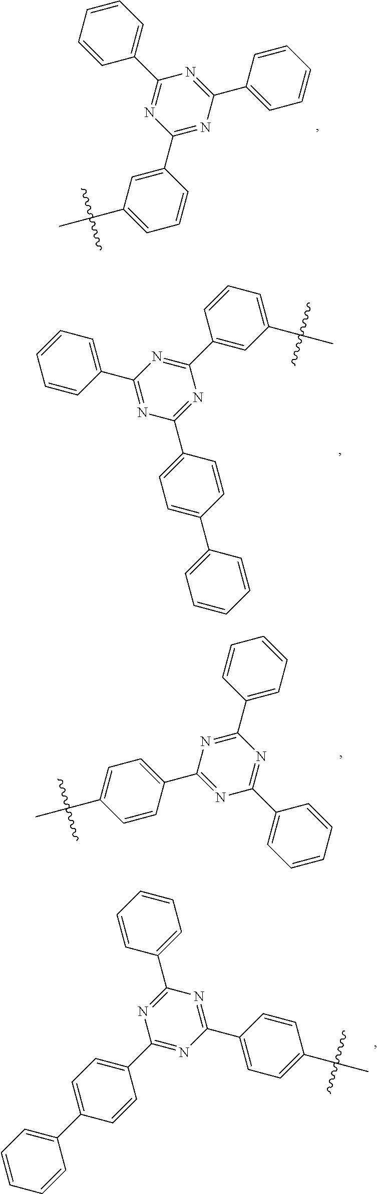

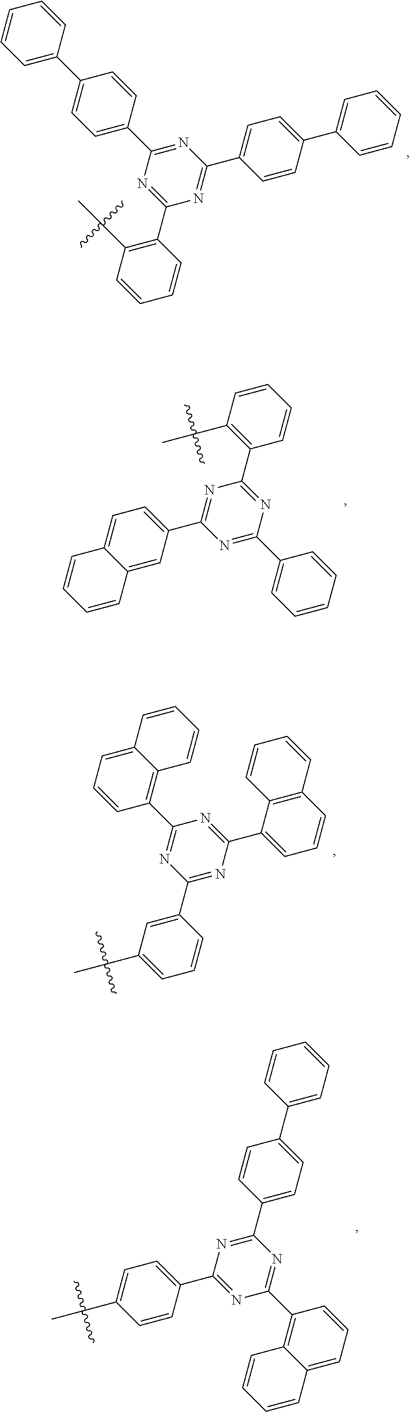 [SMILES: *c1ccccc1-c1nc(-c2ccc(-c3ccccc3)cc2)nc(-c2ccc(-c3ccccc3)cc2)n1 ; *c1ccccc1-c1nc(-c2ccccc2)nc(-c2ccc3ccccc3c2)n1 ; *c1cccc(-c2nc(-c3cccc4ccccc34)nc(-c3cccc4ccccc34)n2)c1 ; *c1ccc(-c2nc(-c3ccc(-c4ccccc4)cc3)nc(-c3cccc4ccccc34)n2)cc1]

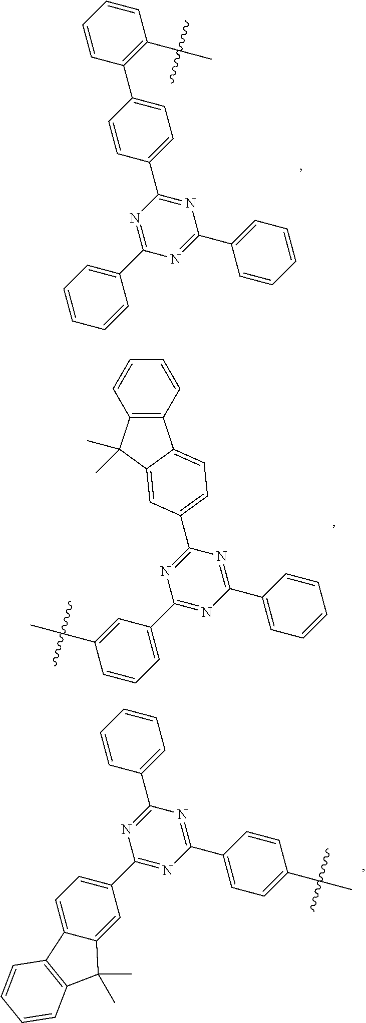

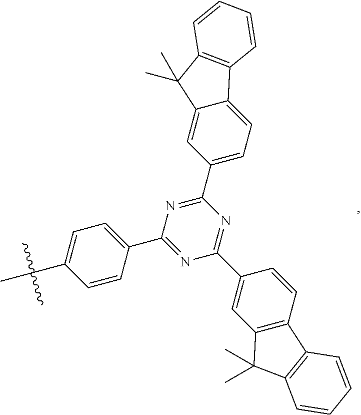 [SMILES: *c1ccc(-c2nc(-c3ccc4c(c3)C(C)(C)c3ccccc3-4)nc(-c3ccc4c(c3)C(C)(C)c3ccccc3-4)n2)cc1]

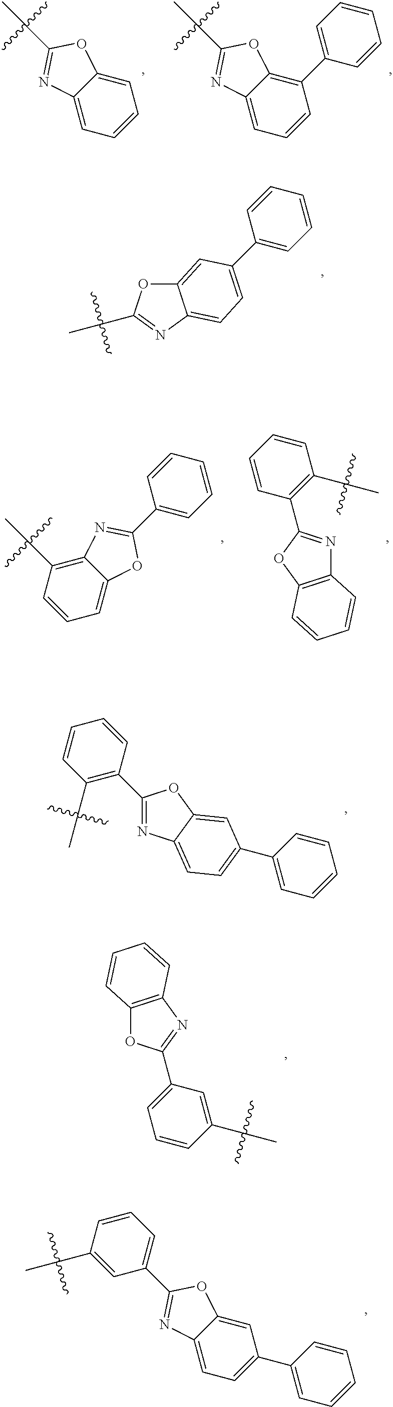

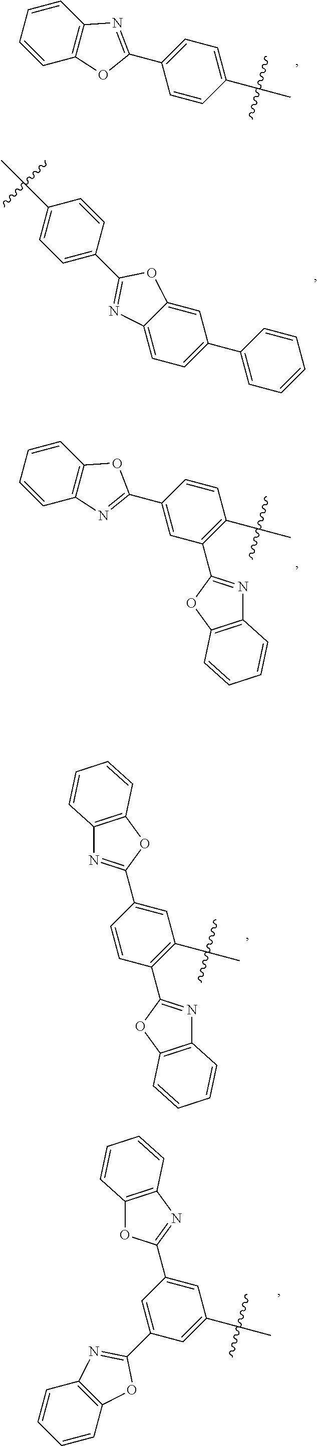 [SMILES: *c1ccc(-c2nc3ccccc3o2)cc1 ; *c1ccc(-c2nc3ccc(-c4ccccc4)cc3o2)cc1 ; *c1ccc(-c2nc3ccccc3o2)cc1-c1nc2ccccc2o1 ; *c1cc(-c2nc3ccccc3o2)ccc1-c1nc2ccccc2o1 ; *c1cc(-c2nc3ccccc3o2)cc(-c2nc3ccccc3o2)c1]

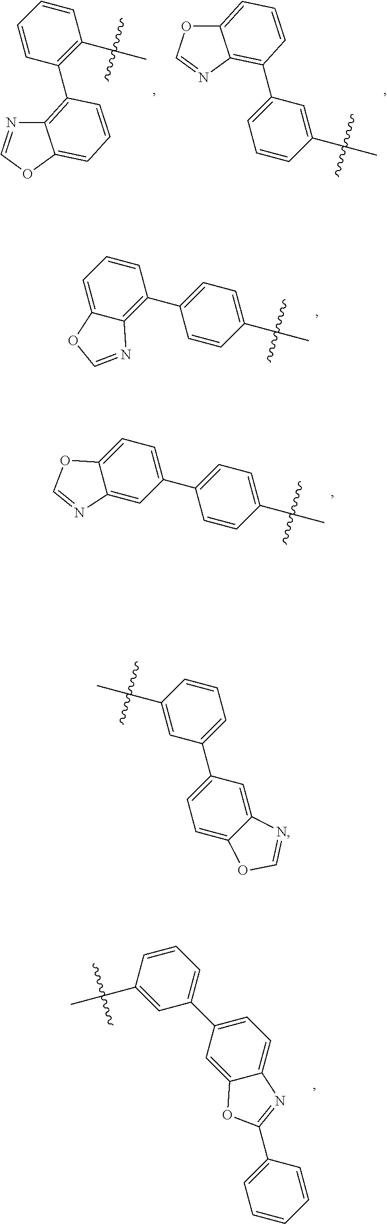 [SMILES: *c1ccccc1-c1cccc2ocnc12 ; *c1cccc(-c2cccc3ocnc23)c1 ; *c1ccc(-c2cccc3ocnc23)cc1 ; *c1ccc(-c2ccc3ocnc3c2)cc1 ; *c1cccc(-c2ccc3ocnc3c2)c1 ; *c1cccc(-c2ccc3nc(-c4ccccc4)oc3c2)c1]

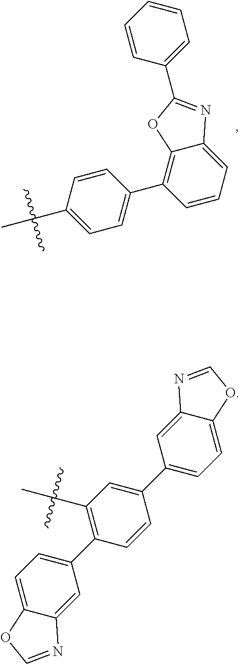 [SMILES: *c1ccc(-c2cccc3nc(-c4ccccc4)oc23)cc1 ; *c1cc(-c2ccc3ocnc3c2)ccc1-c1ccc2ocnc2c1]

Unless otherwise specified, the above-mentioned groups and substituents have ordinary meanings in the field of organic chemistry.

In various parts of the present disclosure, substituents of the compounds disclosed herein are disclosed by group types or ranges. In particular, the present invention includes each and every individual subcombination of the individual members of these group types and ranges. For example, the term “C₁-C₆ alkyl” refers specifically to the independently disclosed methyl, ethyl, C₃ alkyl, C₄ alkyl, C₅ alkyl and C₆ alkyl.

In the present disclosure, unless otherwise expressly stated, the description “ . . . each independently selected from” used throughout the present disclosure may refer to that the specific options expressed by the same or different symbols in different groups do not affect each other, and also that the specific options expressed by the same or different symbols in the same group do not affect each other.

In the present disclosure, the term “C₁-C₆ alkyl” refers to an alkyl having 1 to 6 carbon atoms, preferably “C₁-C₄ alkyl”, more preferably “C₁-C₃ alkyl”, most preferably “C₁-C₂ alkyl”. Examples of “C₁-C₆ alkyl” include, but are not limited to, methyl, ethyl, propyl (e.g., n-propyl, isopropyl), butyl (e.g., n-butyl, isobutyl, tert-butyl), pentyl (e.g., n-pentyl, isopentyl, neopentyl), etc. Examples of “C₁-C₄ alkyl” include, but are not limited to, methyl, ethyl, propyl (e.g., n-propyl, isopropyl), butyl (e.g., n-butyl, isobutyl, tert-butyl), etc. Examples of “C₁-C₃ alkyl” include methyl, ethyl, propyl (e.g., n-propyl, isopropyl), etc. Examples of “C₁-C₂ alkyl” include methyl, ethyl.

In the present disclosure, the terms “aryl” and “aromatic ring” are used interchangeably and refer to that all carbon atoms in each carbocyclic ring have p orbitals that form a conjugated system, for example, in the cases of phenyl and naphthyl; and two or more rings can be connected through σ bonds, for example, in the cases of biphenyl, terphenyl, tetraphenyl, etc. For example, “C₆-C₁₂ aryl/aromatic ring” can be phenyl/benzene ring, naphthyl/naphthane ring, biphenyl/biphenyl ring, and “C₆-C₁₈ aryl/aromatic ring” can be phenyl/benzene ring, naphthyl/naphthane ring, biphenyl/biphenyl ring, terphenyl/terphenyl ring, anthracenyl/anthracene ring, phenanthrenyl/phenanthrene ring, etc.

In the present disclosure, the term “arylene” refers to a divalent group obtained by losing one hydrogen atom from any of the above-mentioned aryl groups or losing two hydrogen atoms from the above-mentioned aromatic ring. For example, “C₆-C₁₂ arylene” can be phenylene, naphthylene, biphenylene, “C₆-C₁₈ arylene” can be phenylene, naphthylene, biphenylene, terphenylene, anthracenylene, phenanthrylene, etc.

In the present disclosure, heteroatom refers to N, O, S, or P.

In the present disclosure, the terms “heteroaryl” and “heteroaromatic ring” are used interchangeably and refer to an aromatic 5- or 6-membered monocyclic group, 8-, 9- or 10-membered bicyclic group and 11- to 14-membered tricyclic group or multicyclic group, in which at least one ring has at least one heteroatom (N, O, S, or P), and the heteroatom-containing ring optionally also has 1, 2 or 3 heteroatoms selected from N, O, S or P. For bicyclic, tricyclic or multicyclic heteroaryl or heteroaromatic ring, it is required that the whole bicyclic, tricyclic or multicyclic structure forms an aromatic system. The heteroaryl or heteroaromatic ring can be connected at any available nitrogen or carbon atom of any ring.

Exemplary monocyclic heteroaryl or monocyclic heteroaromatic ring includes, but is not limited to: pyrrolyl/pyrrole ring, pyrazolyl/pyrazole ring, imidazolyl/imidazole ring, oxazolyl/oxazole ring, isoxazolyl/isoxazole ring, thiazolyl/thiazole ring, thiadiazolyl/thiadiazole ring, isothiazolyl/isothiazole ring, furyl/furane ring, thienyl/thiophene ring, oxadiazolyl/oxadiazole ring, pyridinyl/pyridine ring, pyrazinyl/pyrazine ring, pyrimidinyl/pyrimidine ring, pyridazinyl/pyridazine ring, triazinyl/triazine ring, triazolyl/triazole ring, pyridazinyl/pyridazine ring, 2-pyridone, etc.

Exemplary bicyclic heteroaryl includes, but is not limited to: indolyl, 5-azaindolyl, pyrro[2,3-d]pyrimidinyl, 5,6-diazaindolyl, 6-azaindolyl, 7-azaindolyl, pyrazolo[3,4-b]pyridinyl, pyrro[2,3-c]pyridazinyl, thieno[2,3-d]imidazolyl, thieno[2,3-d]imidazolyl, pyrazolo[3,4-c]pyridinyl, benzothiazolyl, benzimidazolyl, benzoxazolyl, benzisoxazolyl, benzoisothiazolyl, indazolyl, benzothienyl, quinolyl, isoquinolyl, benzofuranyl, indolizinyl, quinoxalinyl, pyrrolopyrimidinyl, furopyridinyl, isoindolyl, et al.

In the present disclosure, the term “heteroarylene” refers to a divalent group obtained by losing one hydrogen atom from any of the above-mentioned heteroaryl groups or losing two hydrogen atoms from any of the above-mentioned heteroaromatic rings.

In the present disclosure, for example, “the pyridinyl, pyrimidinyl, triazinyl, benzoxazolyl are each independently optionally substituted by one or two R^{R}”, wherein “each independently” refers to that the substituents of pyridinyl, pyrimidinyl, triazinyl and benzoxazolyl do not affect each other and can be the same or different; “optionally” refers to that the pyridinyl, pyrimidinyl, triazinyl, benzoxazolyl may or may not be substituted with a substituent; and, when the pyridinyl, pyrimidinyl, triazinyl, and benzoxazolyl are each independently substituted by more than one (i.e., two) R^{R}, the two substituents selected from R^{R} may be the same or different. Please refer to the foregoing content for understanding of other similar definitions in the present disclosure.

In the present disclosure, in Formula O

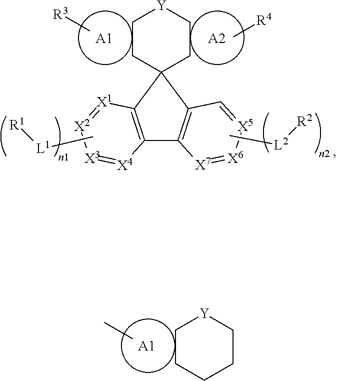

means that Ring A1 is fused with the middle 6-membered ring

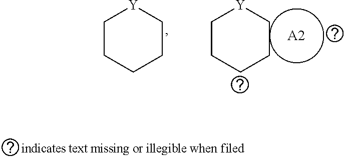

means that Ring A2 is fused with the middle 6-membered ring

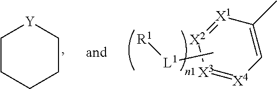

means that the substitution position of the substituent

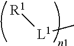

on the ring

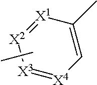

is variable, and those skilled in the art can understand that when

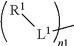

is at a certain position such as X¹ position for substitution, X¹ cannot be N, but CH, and the position of the hydrogen atom in the “CH” will be replaced by

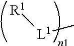

accordingly. Please refer to the foregoing content for understanding of other similar definitions in the present disclosure.

In one aspect of the present disclosure, an electron transport material is provided, which comprises the aforementioned compound, or a stereoisomer or tautomer of the compound.

In another aspect of the present disclosure, an organic electroluminescent device is provided, which comprises an electron transport layer, and the material of the electron transport layer comprises the aforementioned compound, or a stereoisomer or tautomer of the compound.

In some embodiments, the organic electroluminescent device comprises a first electrode, a hole injection layer, a hole transport layer, an electron blocking layer, a light emitting layer, a hole blocking layer, an electron transport layer, an electron injection layer and a second electrode that are stacked in sequence, in which the material of the electron transport layer comprises the aforementioned compound, or a stereoisomer or tautomer of the compound.

In the present disclosure, there is provided an exemplary organic electroluminescent device, as shown in FIG. 1, including: a base substrate 1, a first electrode 2, a hole injection layer 3, a hole transport layer 4, an electron blocking layer 5, a light emitting layer 6, a hole blocking layer 7, an electron transport layer 8, an electron injection layer 9, a second electrode 10, which are stacked in sequence.

In the present disclosure, as an example, the first electrode may be transparent oxide ITO, IZO, or a composite electrode formed such as ITO/Ag/ITO, Ag/IZO, CNT/ITO, CNT/IZO, etc.; the second electrode may be a silver-magnesium composite electrode or Al electrode. The electroluminescent device further comprises a substrate 1 disposed on the side of the first electrode 2 far away from the second electrode 10. The substrate 1 can be a transparent rigid or flexible material, such as glass, polyimide, etc., which can be used to realize rigid substrate displays and flexible displays.

The electroluminescent device further comprises a hole injection layer, a hole transport layer, an electron blocking layer and a light emitting layer stacked in sequence on the side of the anode close to the hole blocking layer; and further comprises an electron injection layer arranged on the side of the electron transport layer far away from the hole blocking layer.

The hole injection layer can be an inorganic oxide, such as oxide of molybdenum, titanium, vanadium, rhenium, ruthenium, chromium, zirconium, hafnium, tantalum, silver, tungsten, manganese and other metal, or it can also be a dopant with strong electron-attracting system, such as F4TCNQ, HAT-CN, etc., or P-type doping can be performed on the hole transport material, and the thickness of the hole injection layer can be 5 nm to 30 nm.

The material of the hole transport layer has good hole transport properties and can be aromatic amine or carbazole materials, such as NPB, TPD, BAFLP, DFLDPBi, etc., and the thickness of the hole transport layer can be 300 nm to 100 nm.

The electron blocking layer, that is, the luminescent auxiliary layer, has hole transport properties and can be a red luminescent auxiliary layer, a green luminescent auxiliary layer, or a blue luminescent auxiliary layer. The material of luminescent auxiliary layer can be an aromatic amine or carbazole material, such as CBP, PCzPA, etc., and the thickness of the electron blocking layer can be 5 nm to 50 nm.

The light emitting layer can be phosphorescent host and red phosphorescent dopant, phosphorescent host and green phosphorescent dopant, or fluorescent host and fluorescent dopant. The host material of the light emitting layer can comprise one material or a mixture of two or more materials, wherein the host material of the blue light emitting layer can be selected from anthracene derivatives ADN, MADN, etc., and the guest material can be selected from pyrene derivatives, fluorene derivatives, perylene derivatives, styrylamine derivatives, metal complexes, etc., such as TBPe, BDAVBi, DPAVBi, FIrpic, etc.; the host material of the green light emitting layer can be selected from coumarin dyes, quinacridine copper derivatives, polycyclic aromatic hydrocarbons, diamine anthracene derivatives, carbazole derivatives, such as DMQA, BA-NPB, Alq3, etc., the guest material can be selected from metal complexes, etc., such as Ir(ppy)₃, Ir(ppy)₂(acac), etc.; the host material of the red light emitting layer can be selected from the DCM series materials, such as DCM, DCJTB, DCJTI, etc., and the guest material can be selected from metal complexes, such as Ir(piq)₂(acac), POEP, Ir(btp)₂(acac), etc., and the thickness of the light emitting layer can be 20 nm to 100 nm.

The thickness of the hole blocking layer can be 5 nm to 100 nm, and the thickness of the electron transport layer can be 20 nm to 100 nm. The hole blocking layer and the electron transport layer independently comprise aromatic heterocyclic compounds, for example, imidazole derivatives such as benzimidazole derivatives, imidazopyridine derivatives and benzimidazophenanthridine derivatives, as well as pyrimidine derivatives, triazine derivatives and their analogues, etc., as well as compounds containing a nitrogen-containing 6-membered ring structure, such as quinoline derivatives, isoquinoline derivatives, and phenanthroline derivatives, and may also comprise compounds with phosphine oxide substituents on heterocyclic ring, such as: OXD-7, TAZ, p-EtTAZ, BPhen, BCP, and the electron transport material of the present disclosure, etc.

The thickness of the electron injection layer may be 1 nm to 10 nm, and the electron injection layer material comprises alkali metals or metals, such as LiF, Yb, Mg, Ca or compounds thereof.

In the Example as shown in FIG. 1, the structure of the luminescent device is: substrate/first electrode (ITO)/hole injection layer (5 nm)/hole transport layer (30 nm)/electron blocking layer (10 nm)/light emitting layer (20 nm)/hole blocking layer (10 nm)/electron transport layer (40 nm)/electron injection layer (1 nm)/second electrode (100 nm).

In another aspect of the present disclosure, there is provided an electronic device, which comprises the aforementioned organic electroluminescent device.

### Beneficial Effects

1. The present disclosure provides an electron transport material comprising an azaspirocyclic group and some electron-withdrawing groups, and applies the material to organic electroluminescent devices to improve the performance of the luminescent devices. In the structure of this electron transport material, the benzoxazole group has high electron affinity, giving it excellent electron conduction properties; at the same time, in the process of designing the molecular structure, a variety of electron-withdrawing groups are selected, which have lower LUMO energy levels and can effectively transport electrons into the light emitting layer, increasing the recombination probability of electrons and holes in the light emitting layer, and allowing the device to exhibit high luminous efficiency; the spirocyclic group has an orthogonal spatial configuration, which can reduce the van der Waals force between molecules and help prevent the crystallization of electron transport material; at the same time, the structure is more rigid, which makes the compound have a higher glass transition temperature, improving the film-forming properties and thermal stability of the compound.

2. The present disclosure provides an electron transport material comprising electron-withdrawing groups and an azaspirocyclic group, and applies the material to an organic electroluminescent device. In the structure of the electron transport material, the various electron-withdrawing groups selected have high electron affinity, which gives it excellent electron conduction properties, helps transport effectively electrons into the light emitting layer, allows to increase the recombination probability of electrons and holes in the light emitting layer, and enables the device to exhibit high luminous efficiency; the azaspirocyclic group has an orthogonal spatial configuration, which can reduce the van der Waals force between molecules and help prevent the electron transport material from crystallization; at the same time, the relatively rigid structure enables the compound to have a higher glass transition temperature, improves the film-forming properties and thermal stability of the compound, and further improves the luminescence life and operational stability of organic electroluminescent devices.

3. The present disclosure provides an electron transport material comprising an azaspirocyclic group and some electron-withdrawing groups, and applies the material to organic electroluminescent devices to improve the performance of the luminescent devices. In the structure of this electron transport material, azaphenanthrene groups, benzoxazole groups and azine groups all have high electron affinity, which gives it excellent electronic conduction properties; at the same time, in the process of designing molecular structure, some electron-withdrawing groups are selected, which have lower LUMO energy levels and can effectively transport electrons into the light emitting layer, thereby increasing the recombination probability of electrons and holes in the light emitting layer, and the devices show high luminous efficiency; the spirocyclic group has an orthogonal spatial configuration, which can reduce the van der Waals force between molecules and help prevent the crystallization of electron transport material; at the same time, the structure is relatively rigid and the compound has a higher glass transition temperature, which improves the film-forming properties and thermal stability of the compound. These material properties have significantly improved the use process of the material and the performance of the devices.

4. The present disclosure provides a material comprising an azaspirocyclic group containing a sp3 hybridized carbon atom, and the sp3 hybridized carbon atom has an orthogonal configuration and large steric hindrance, which can reduce the van der Waals force between molecules and can inhibit the crystallization phenomenon of material during film-forming process, and this material is used as an electron transport material and applied to organic electroluminescent devices in order to achieve excellent device performance. In the structure of this electron transport material, the various electron-withdrawing groups selected have high electron affinity, which gives it excellent electron conduction properties, allows electrons to transport effectively into the light emitting layer, increases the probability of recombination of electrons and holes in the light emitting layer, and enables the device to exhibit high luminous efficiency; at the same time, the structure is relatively rigid, which allows the compound to have a higher glass transition temperature, improves the film-forming properties and thermal stability of the compound, and further improves the luminescence life and operational stability of organic electroluminescent devices.

### BRIEF DESCRIPTION OF THE DRAWINGS

FIG. 1 shows an exemplary device structure of an experimental example of the present disclosure, in which: 1: base substrate, 2: first electrode, 3: hole injection layer, 4: hole transport layer, 5: electron blocking layer, 6: light emitting layer, 7: hole blocking layer, 8: electron transport layer, 9: electron injection layer, 10: second electrode.

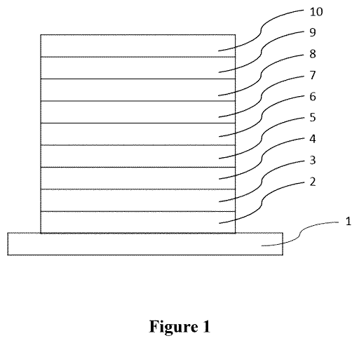

### SPECIFIC MODELS FOR CARRYING OUT THE INVENTION

The present disclosure will be further explained below with reference to specific examples, which should not be construed as limiting the present disclosure in any way.

### Example: Preparation and Physical and Chemical Properties of Compounds

### 1. Preparation of Compounds

In the present disclosure, the compounds shown below were prepared:

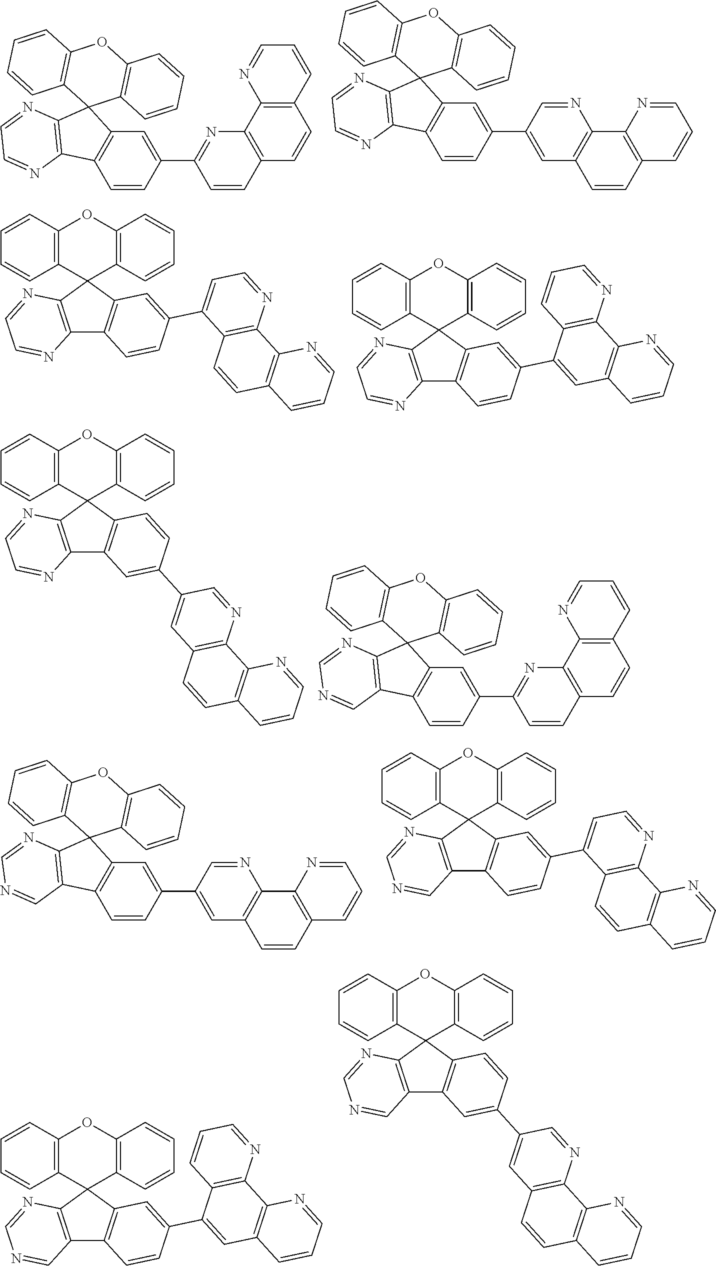

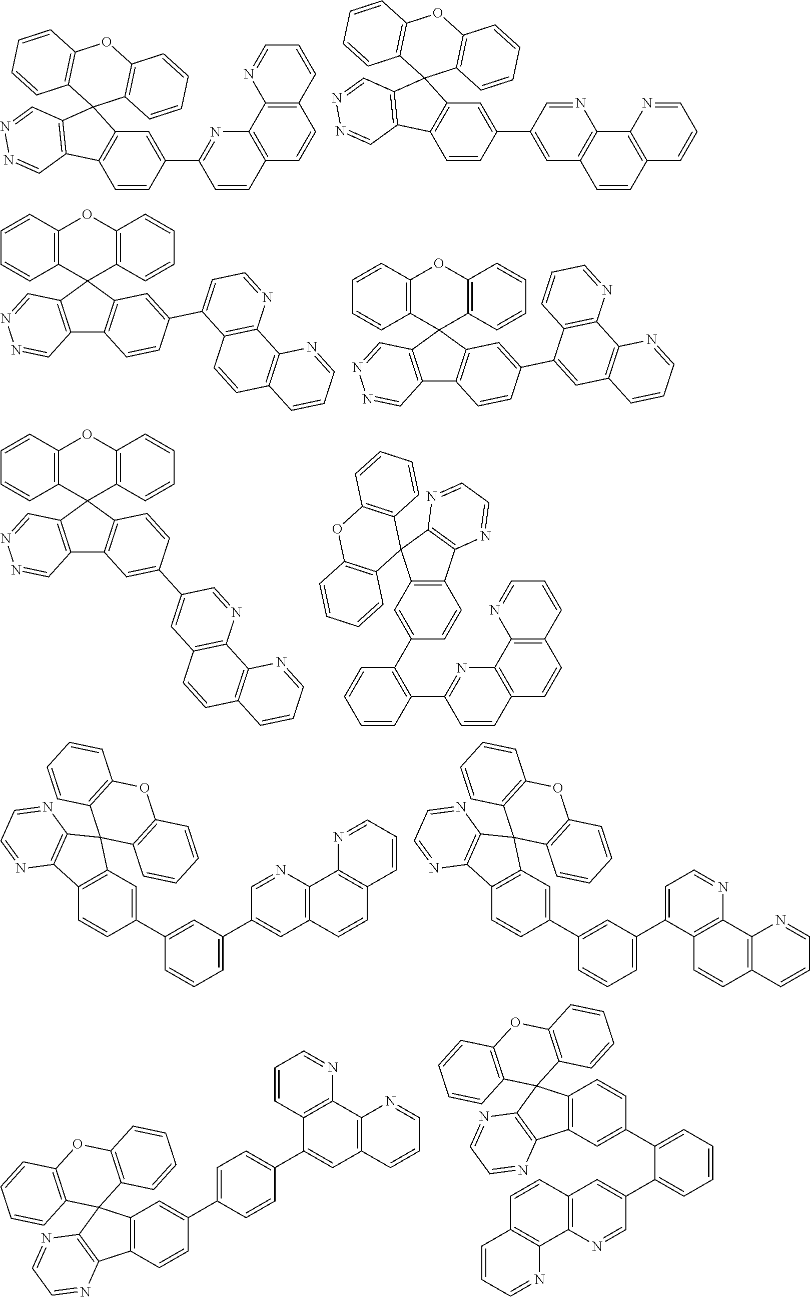

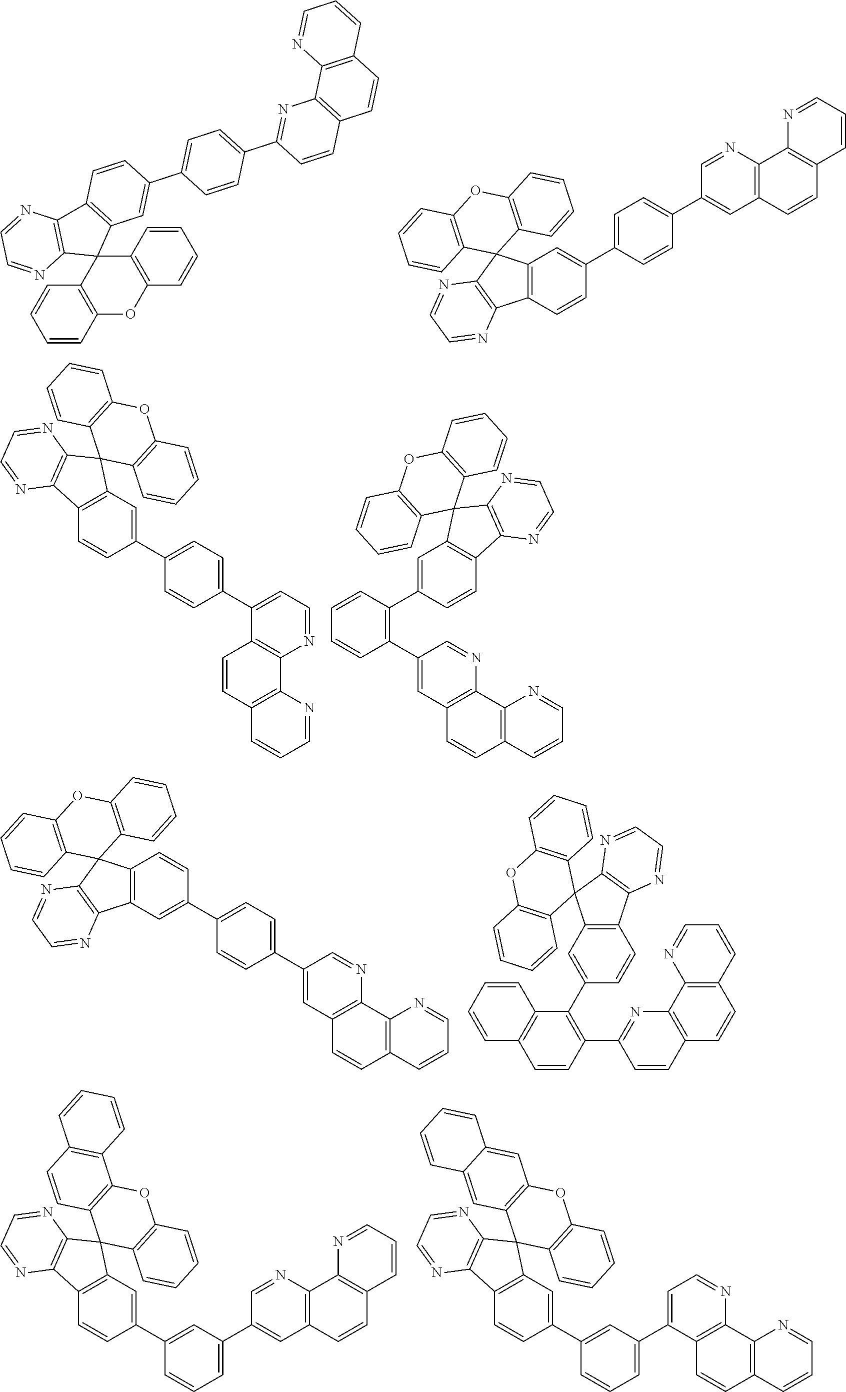

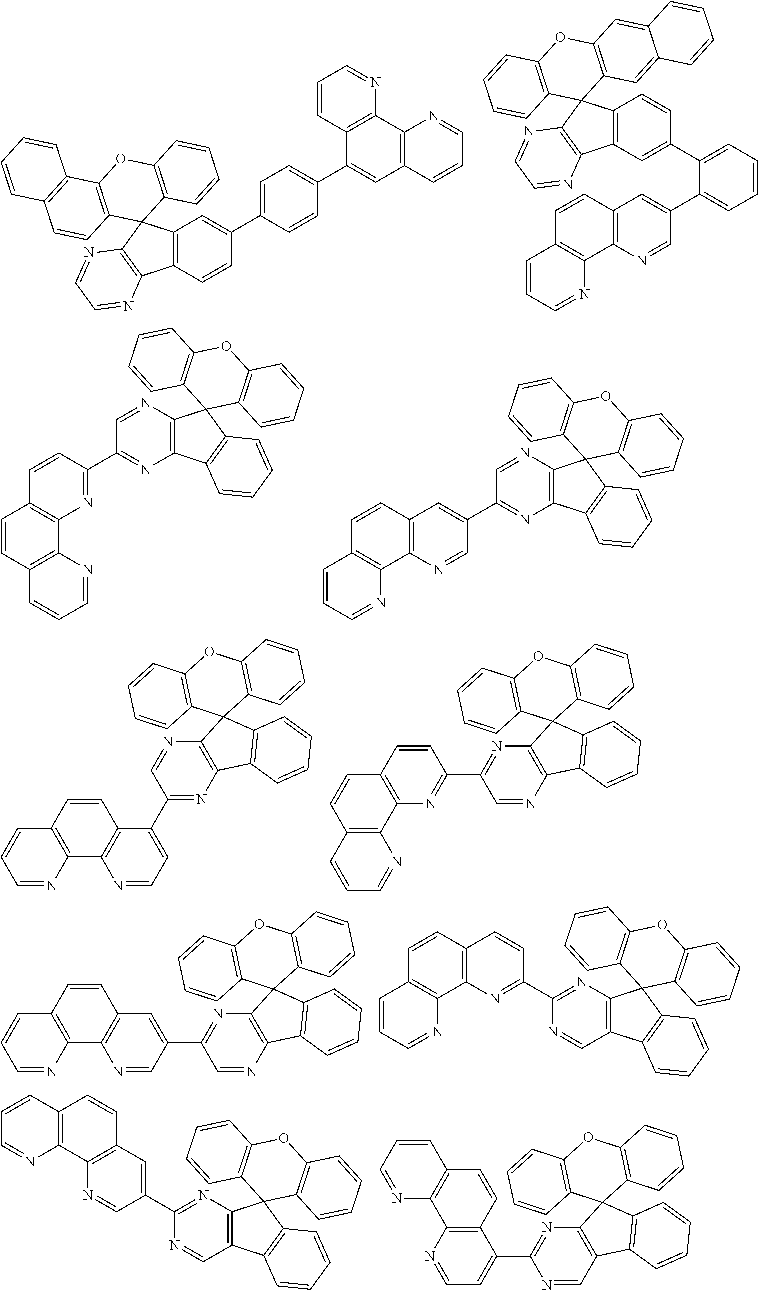

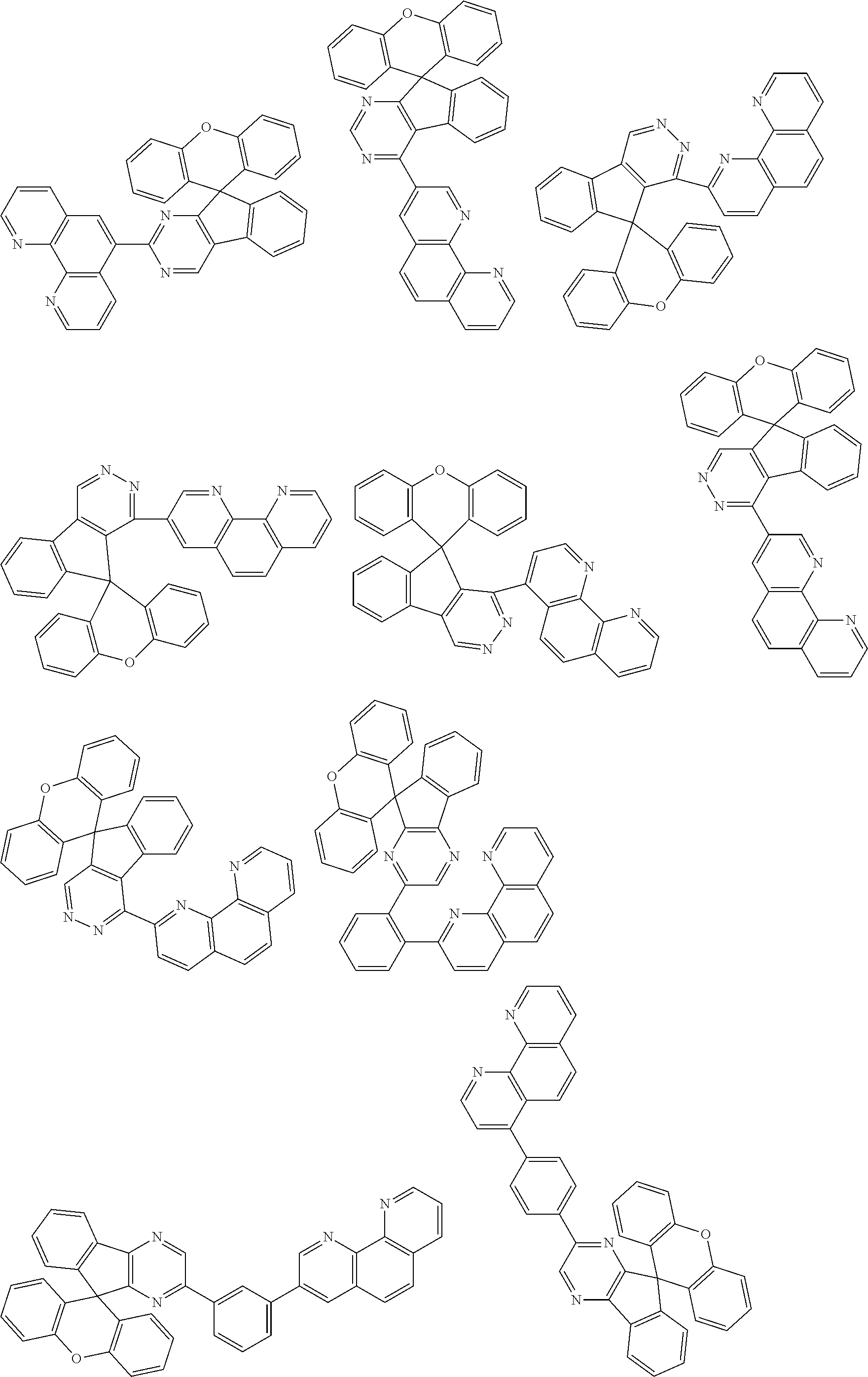

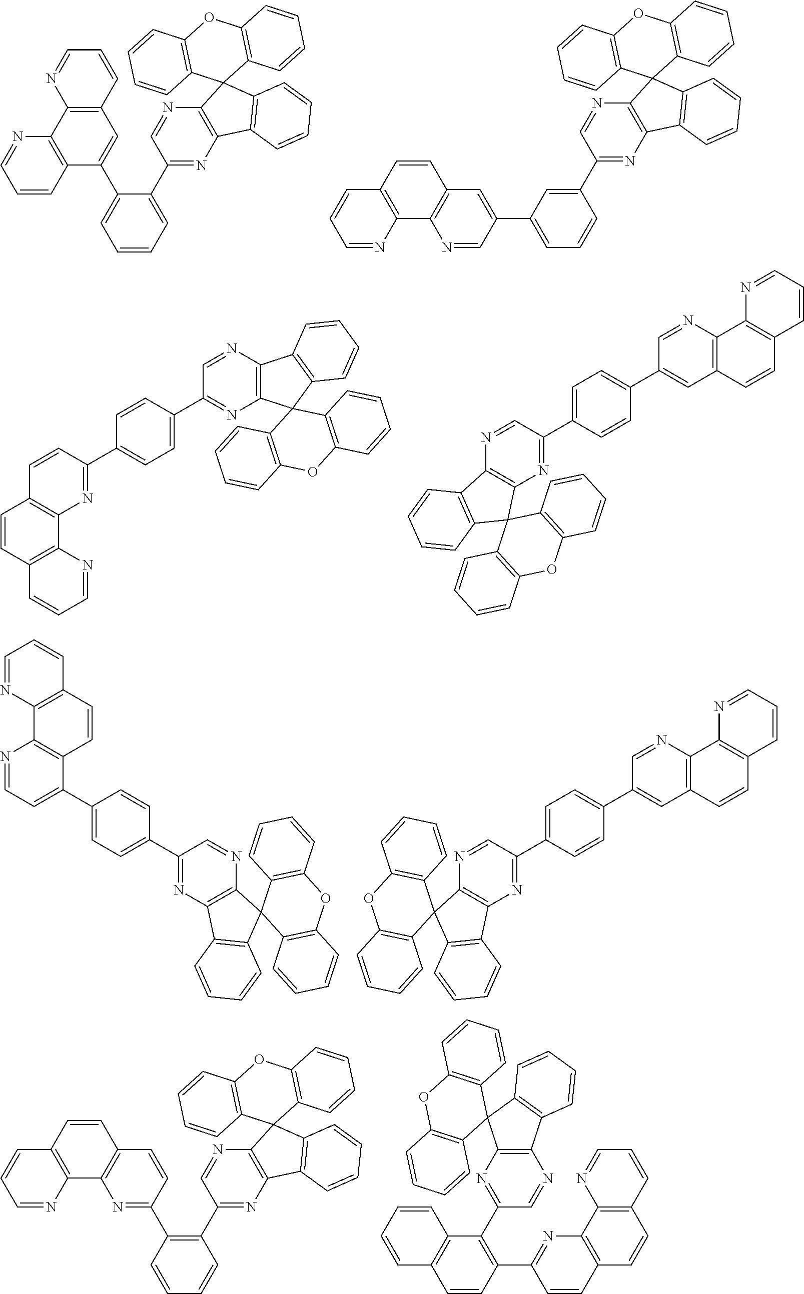

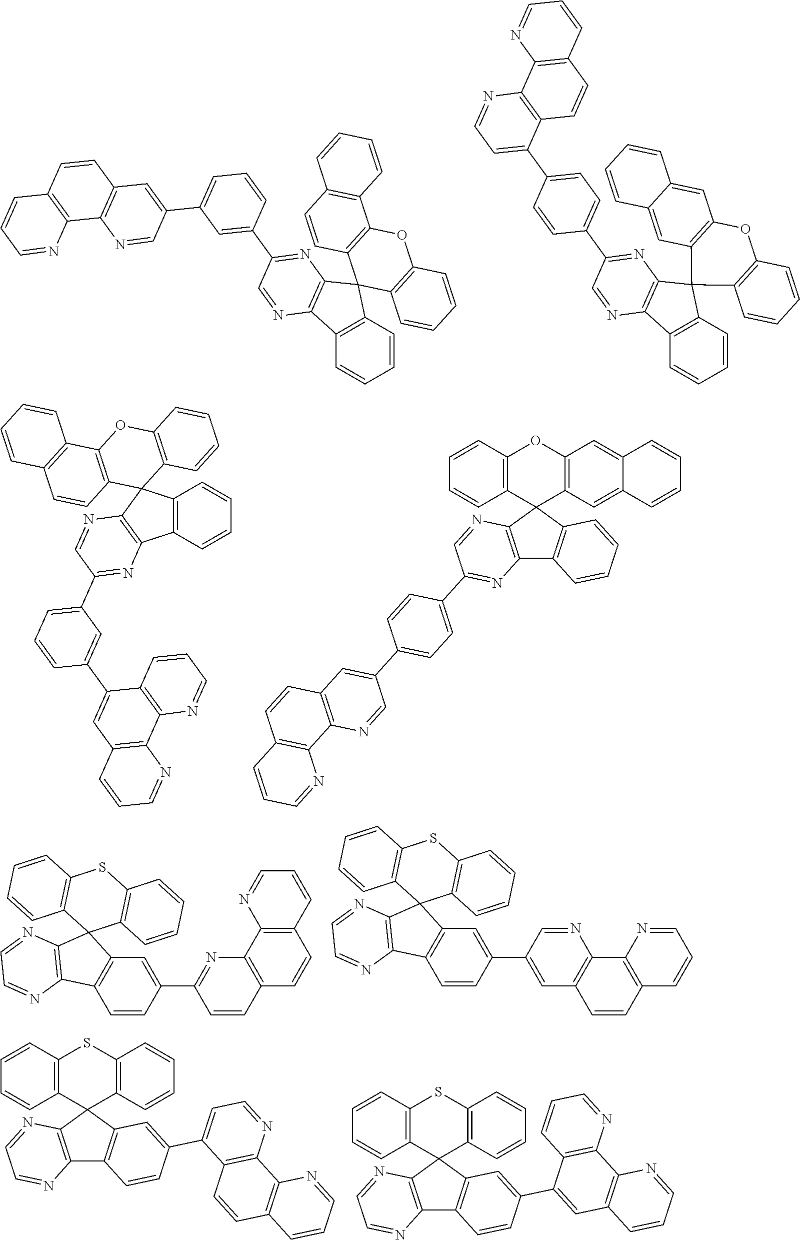

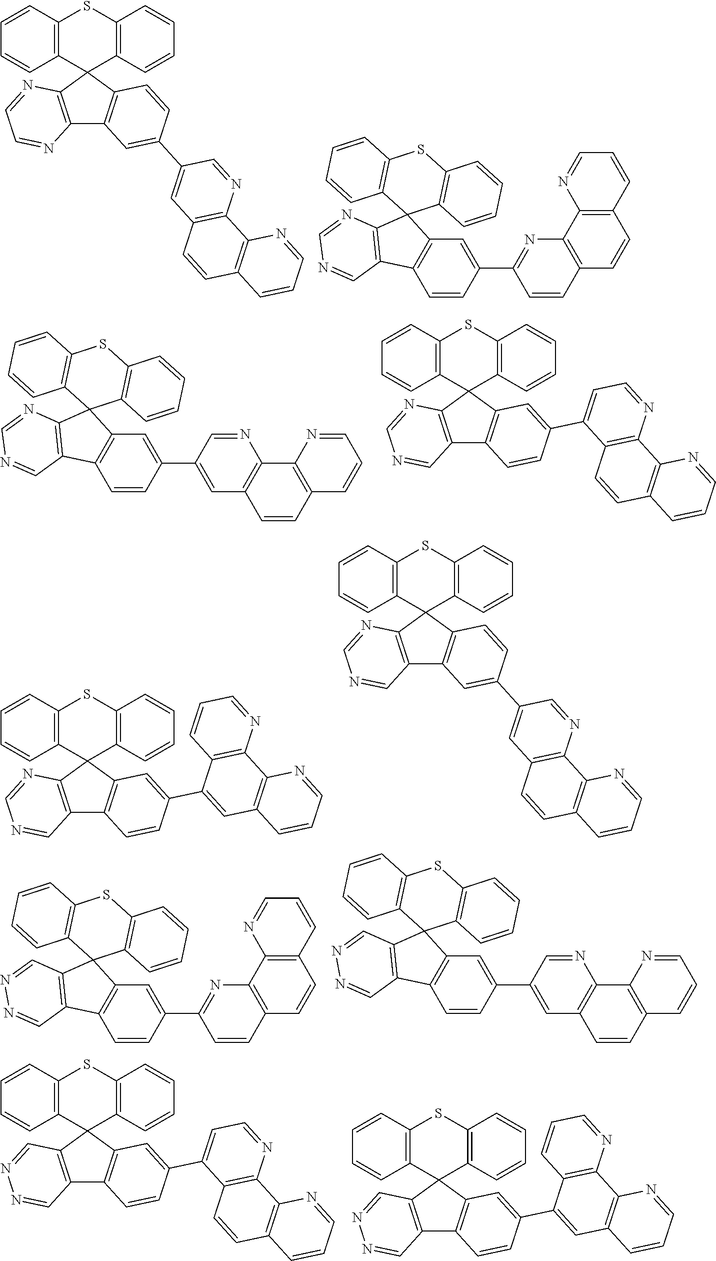

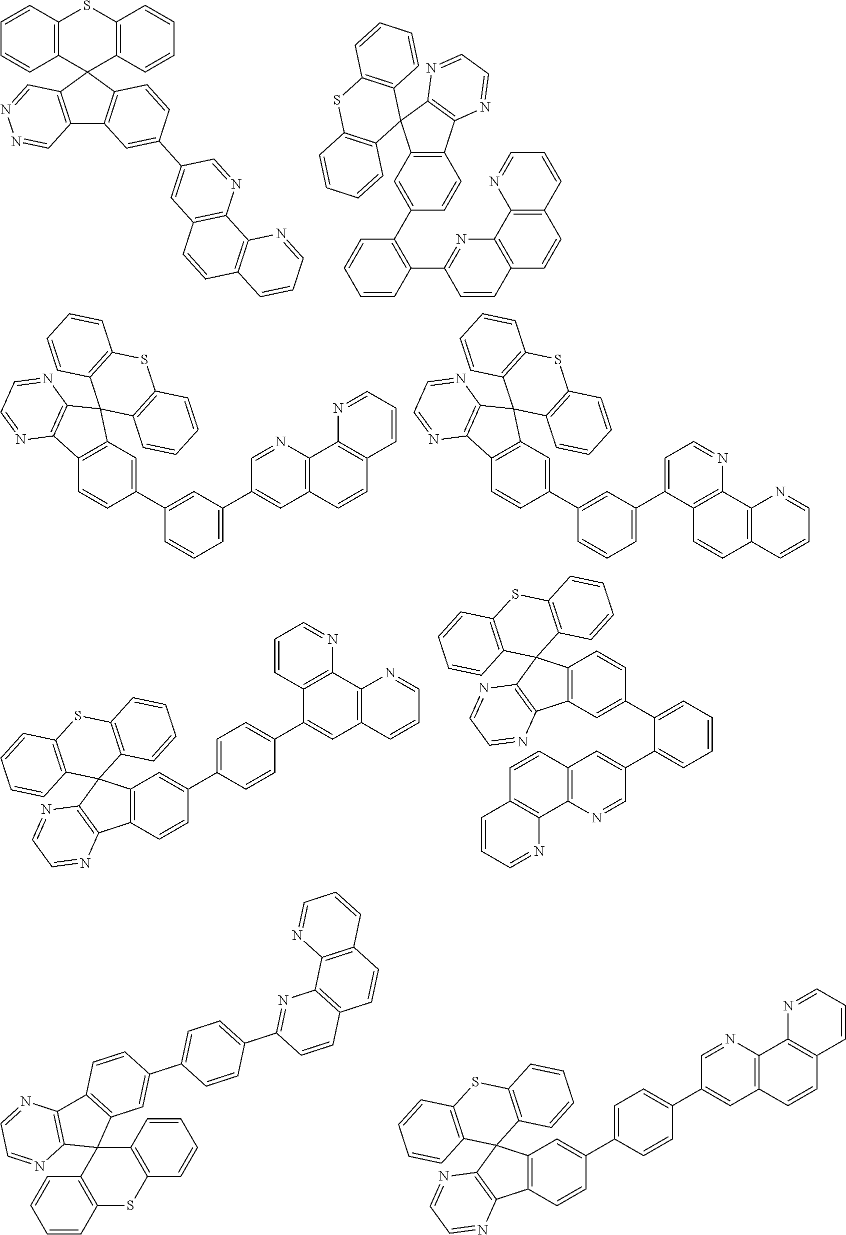

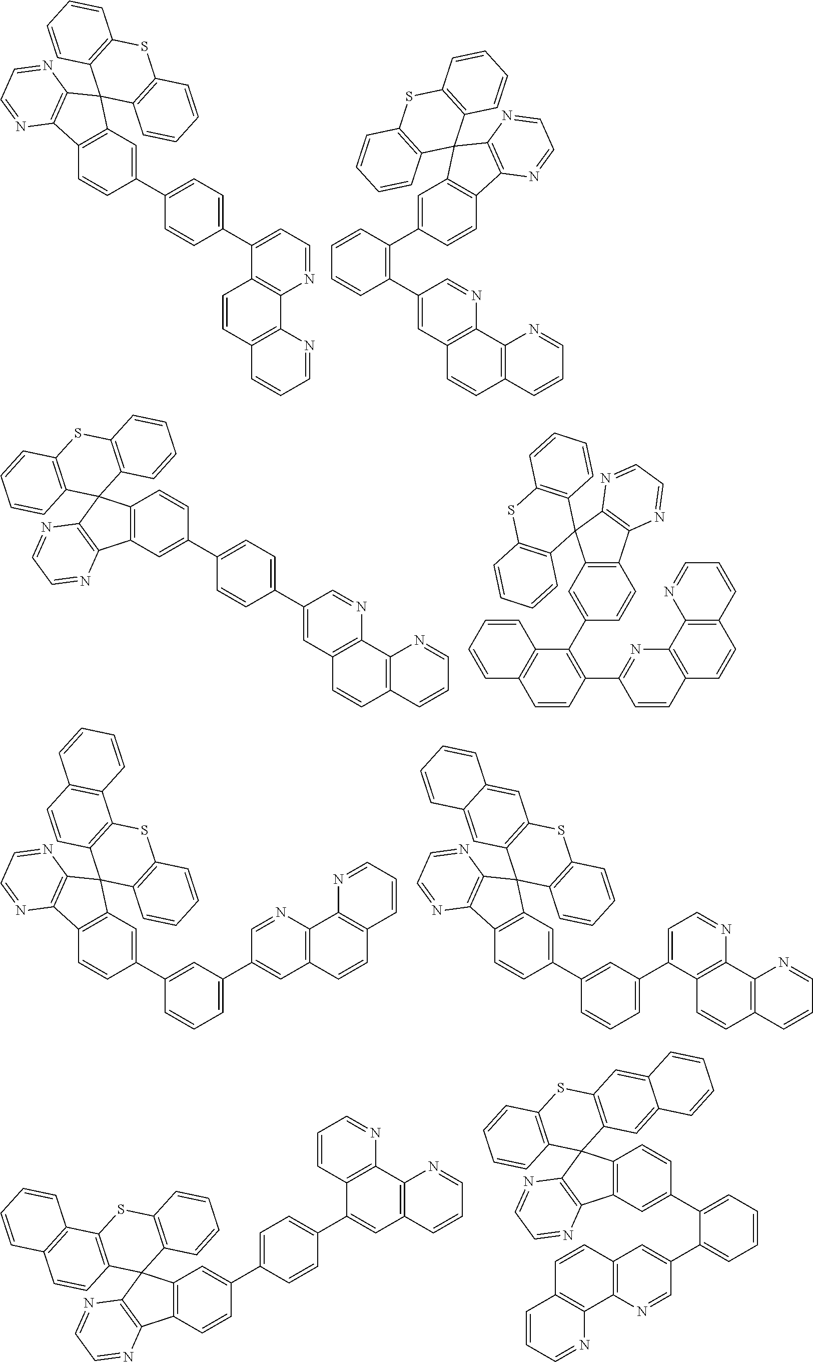

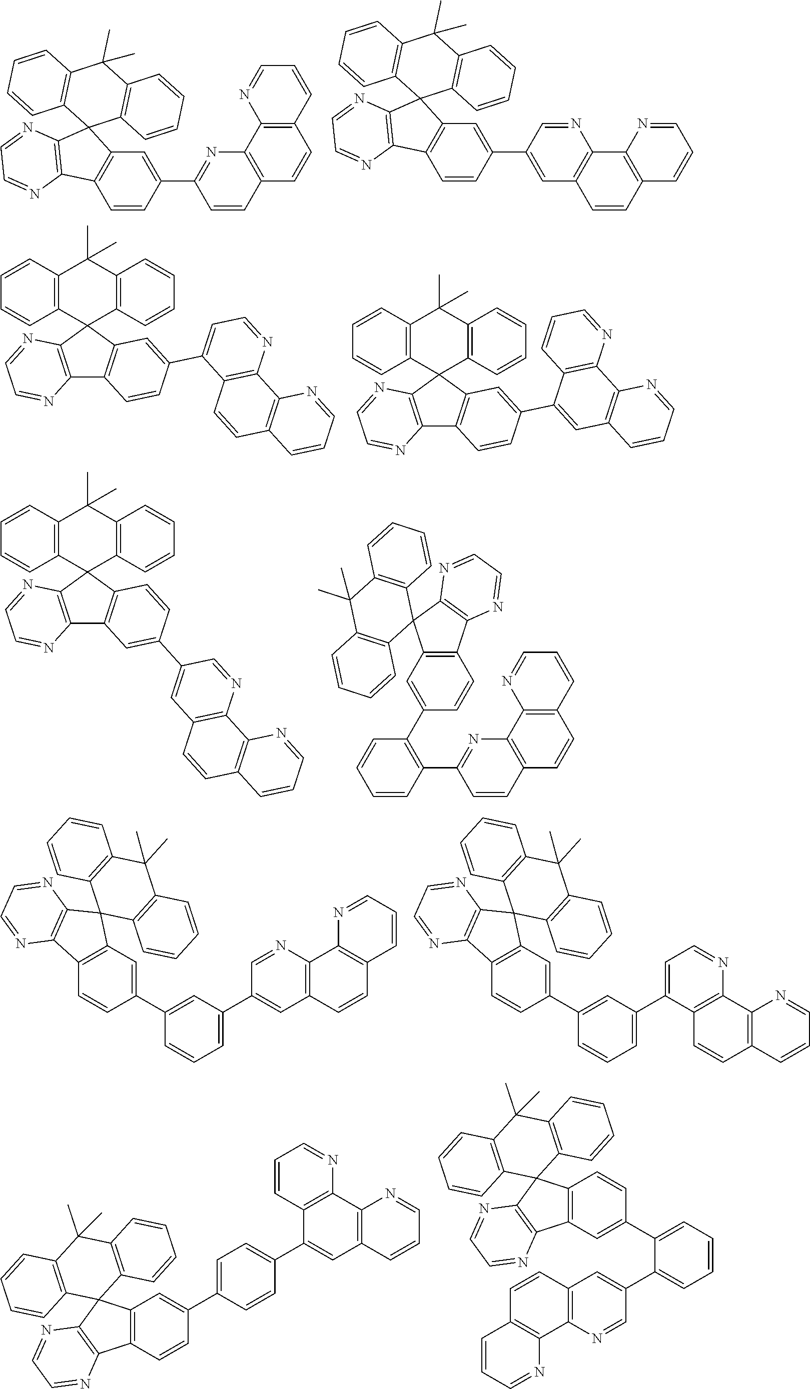

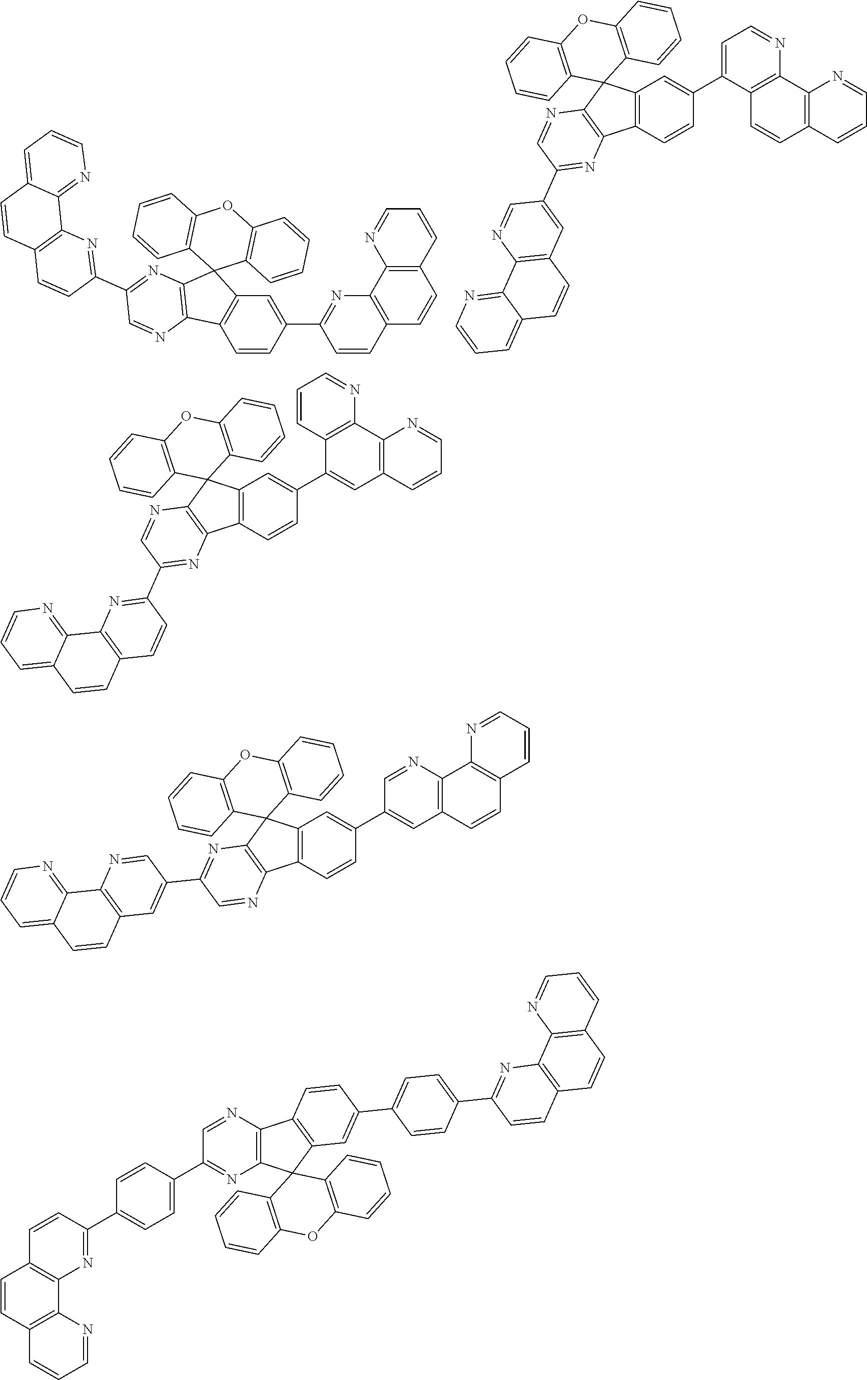

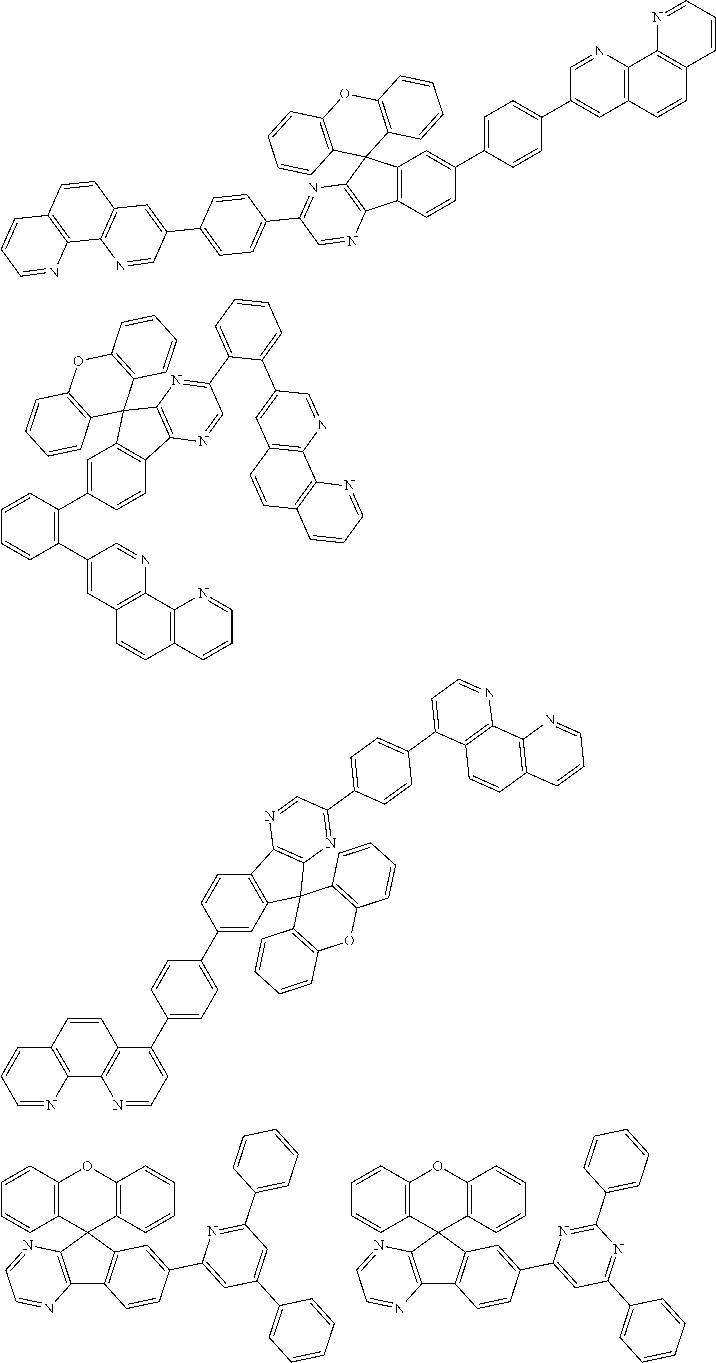

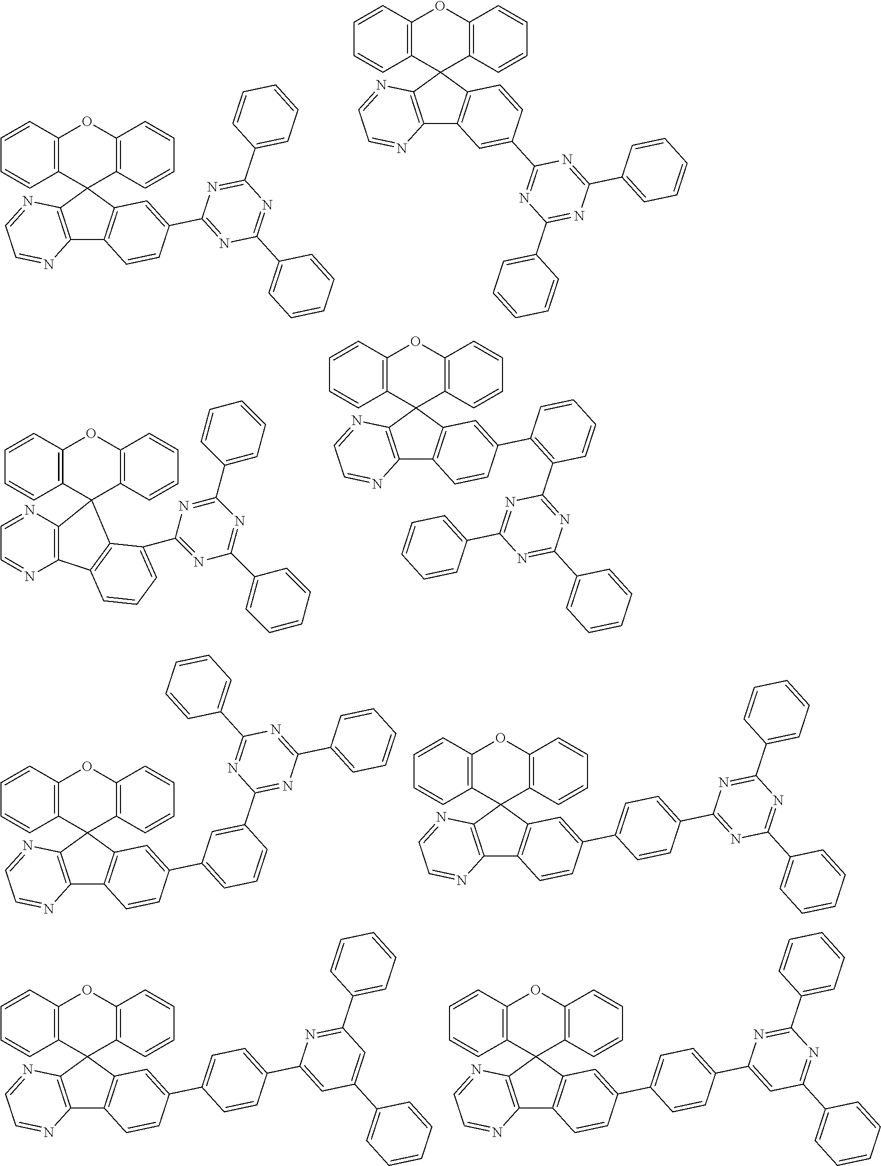

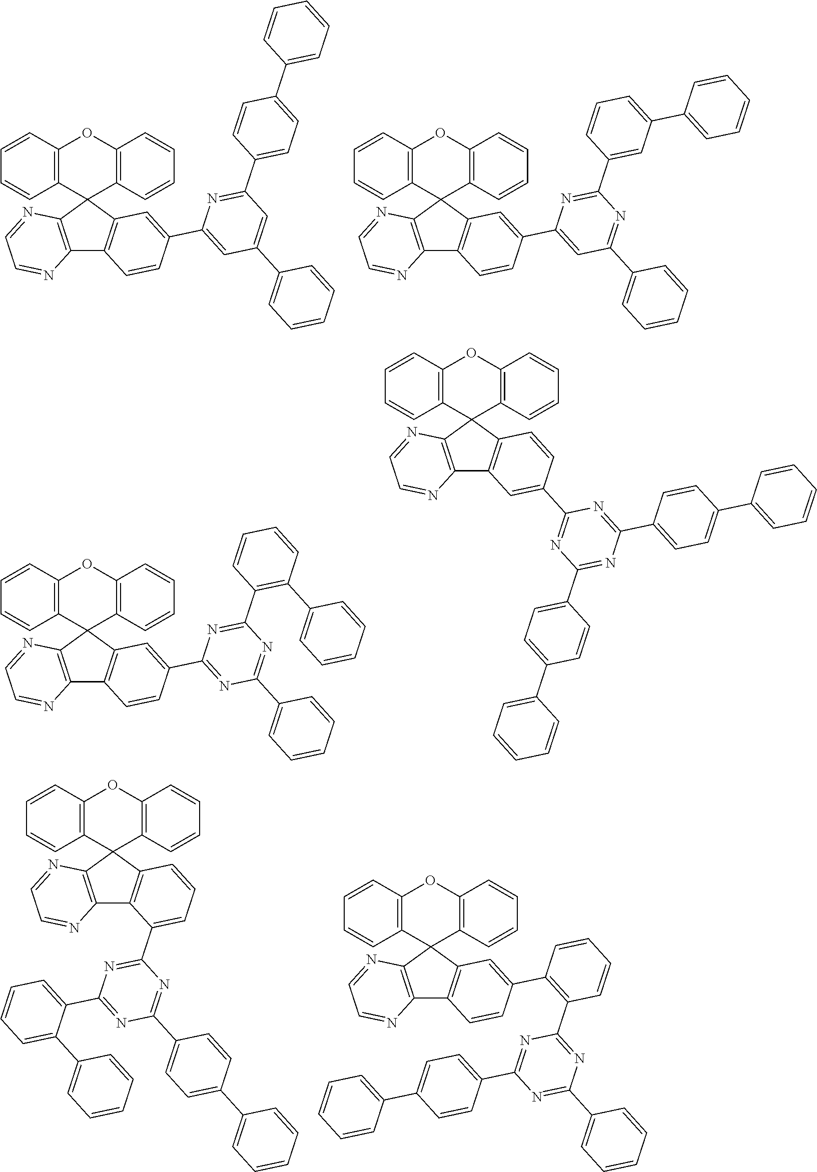

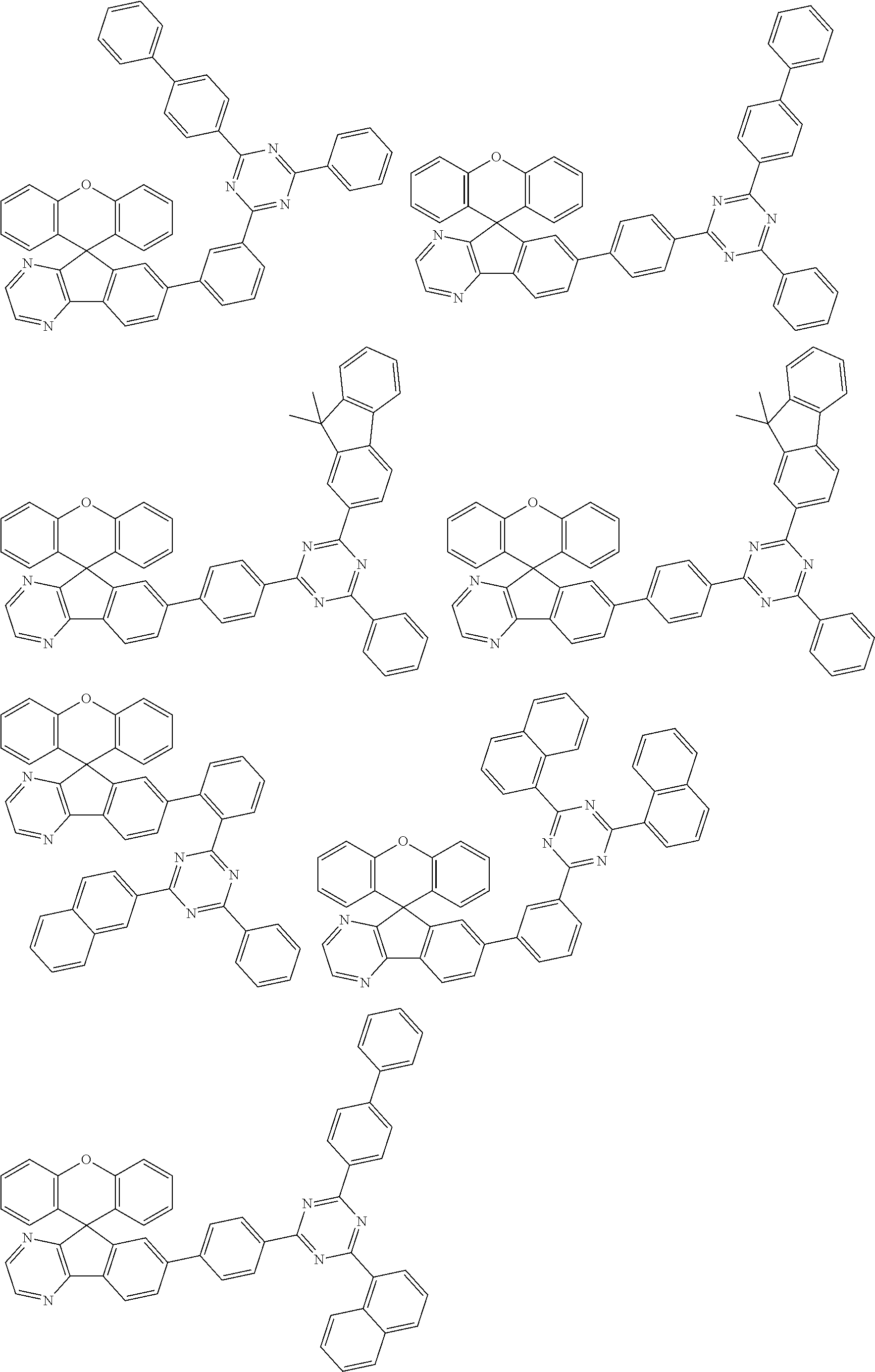

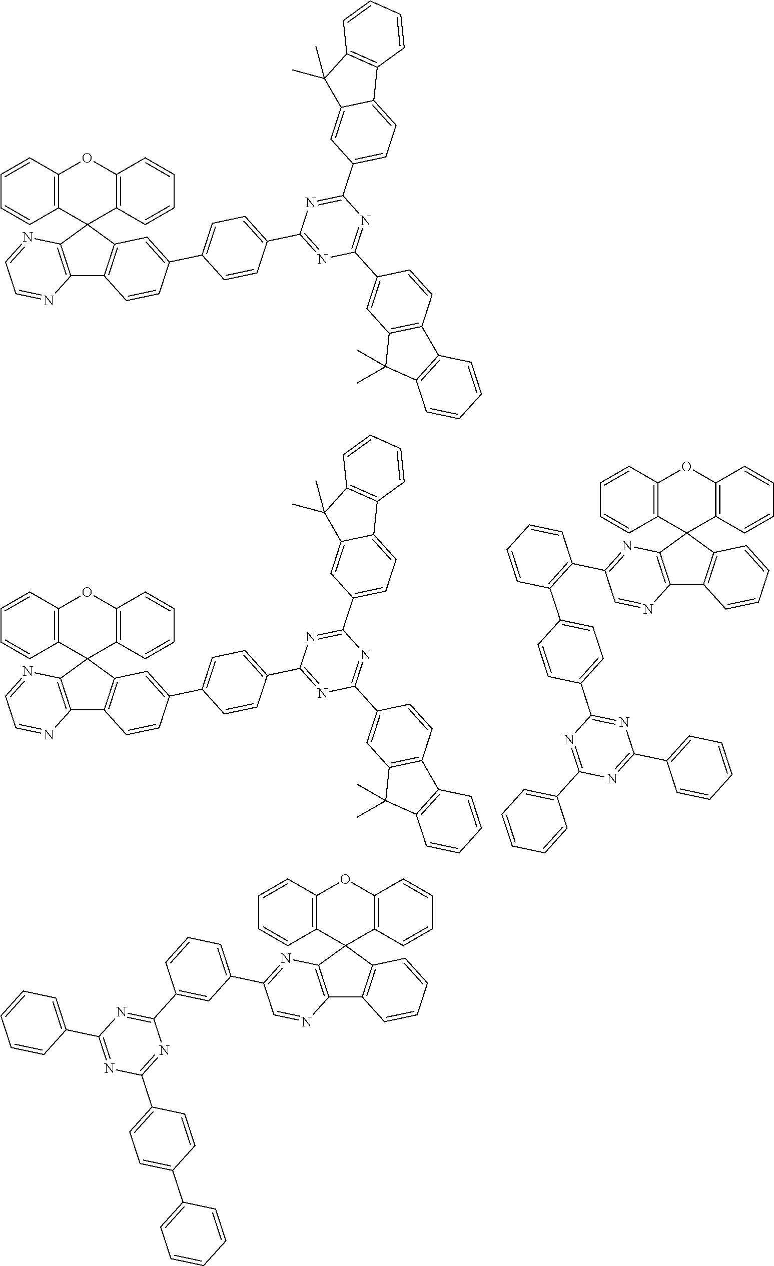

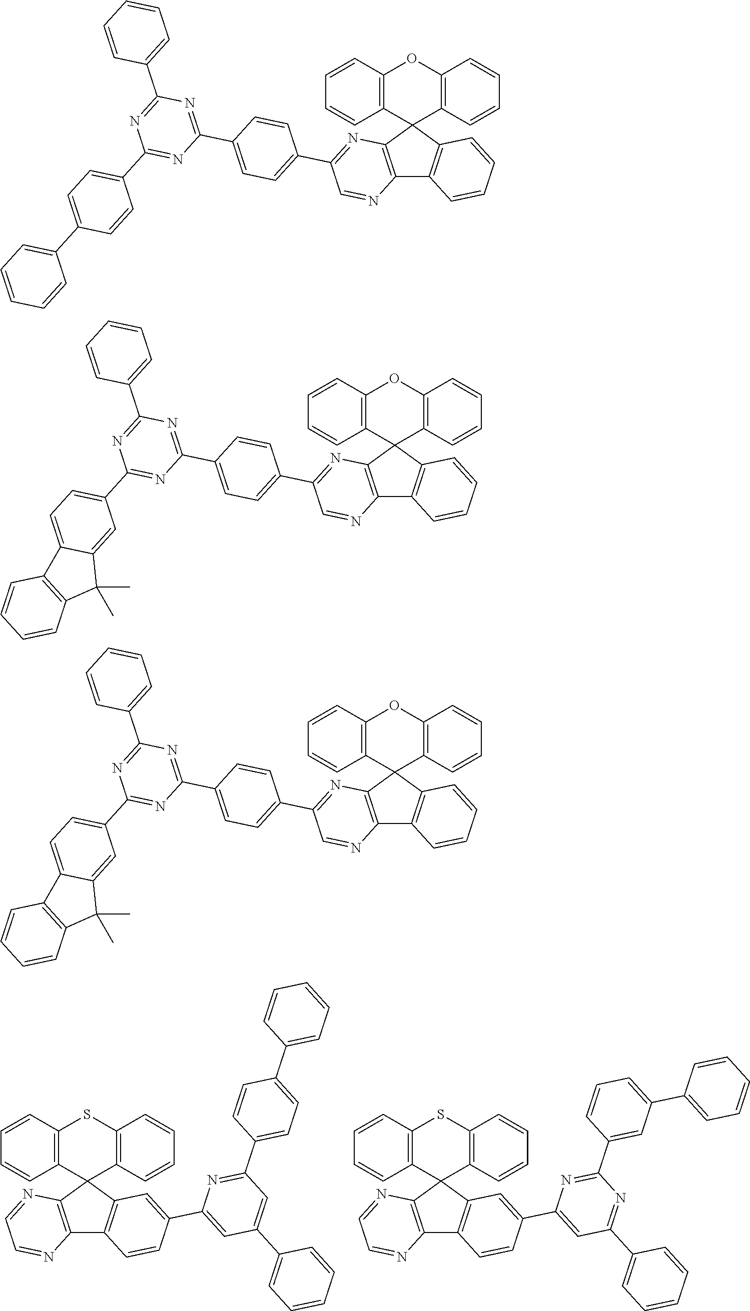

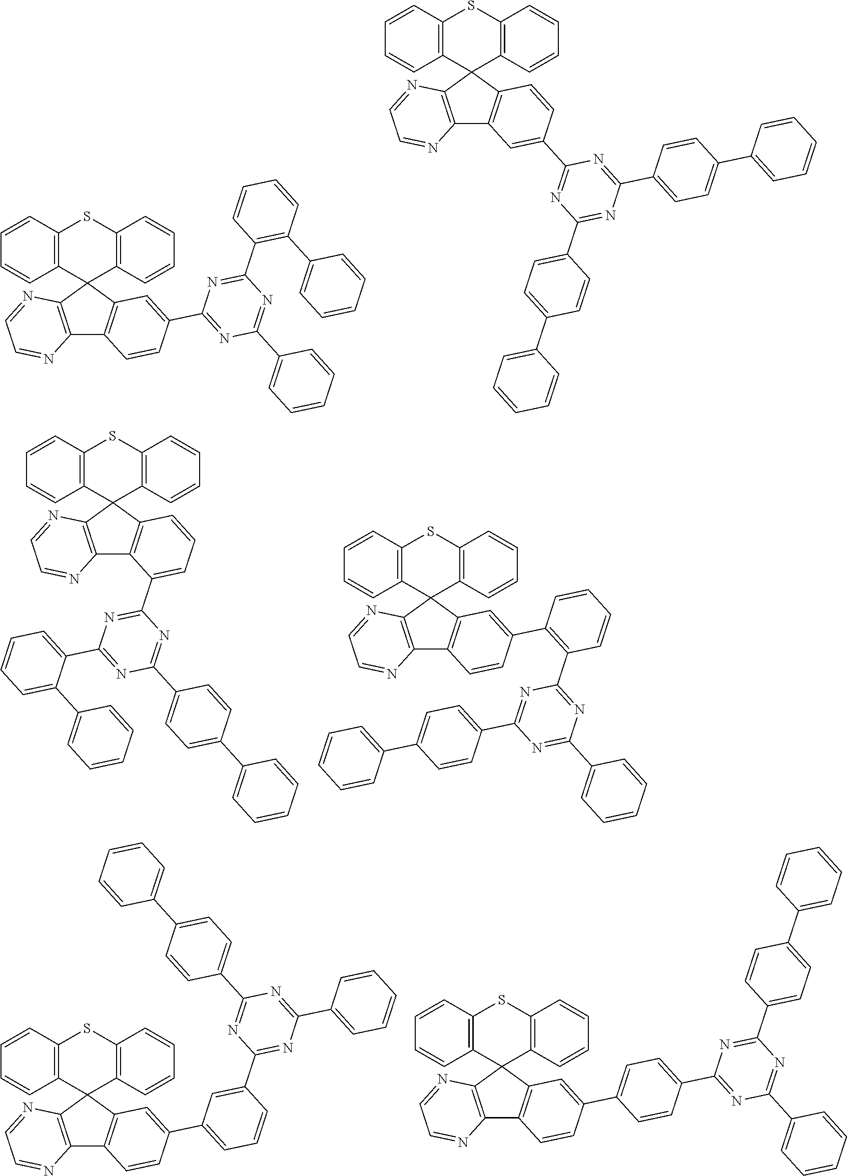

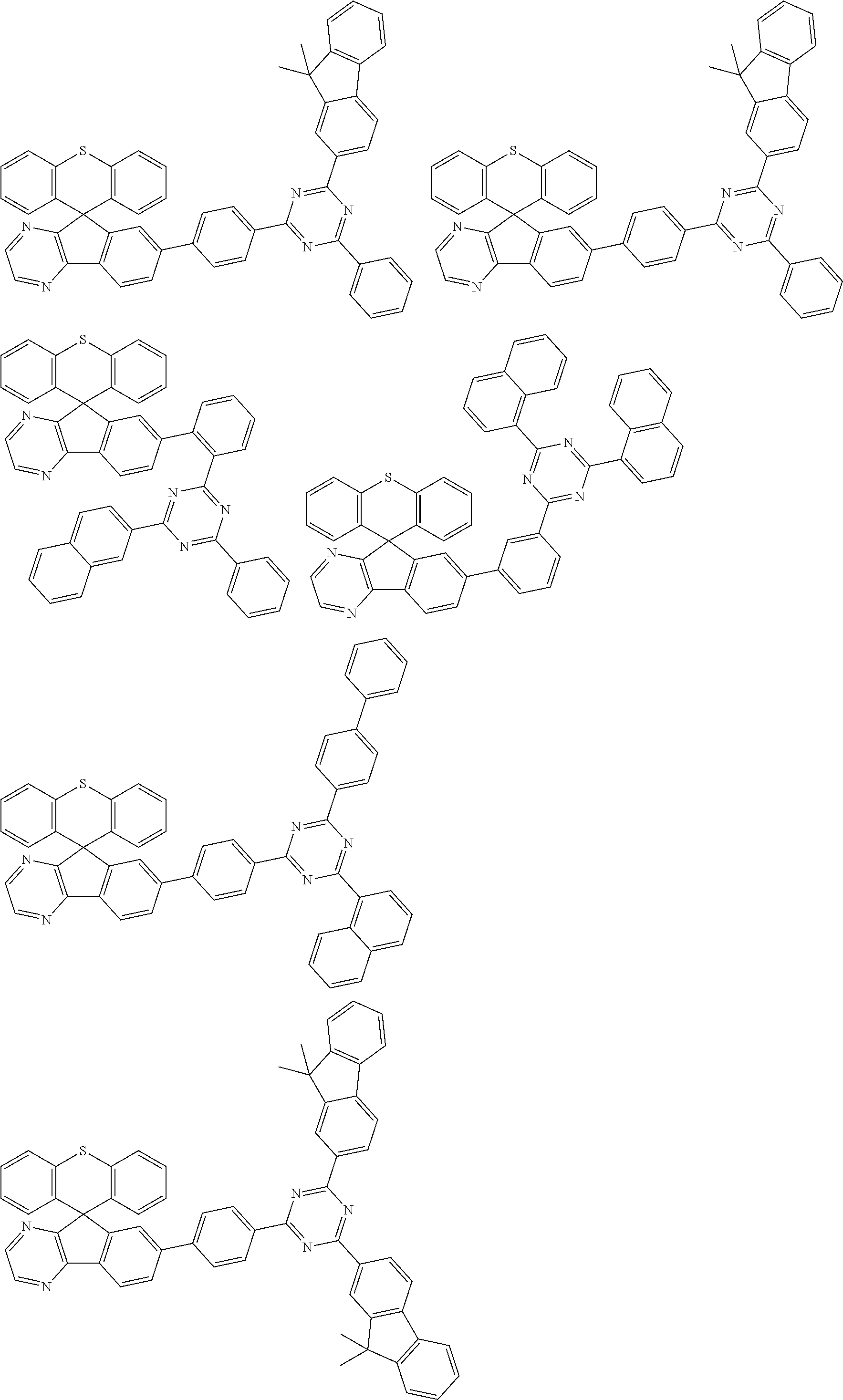

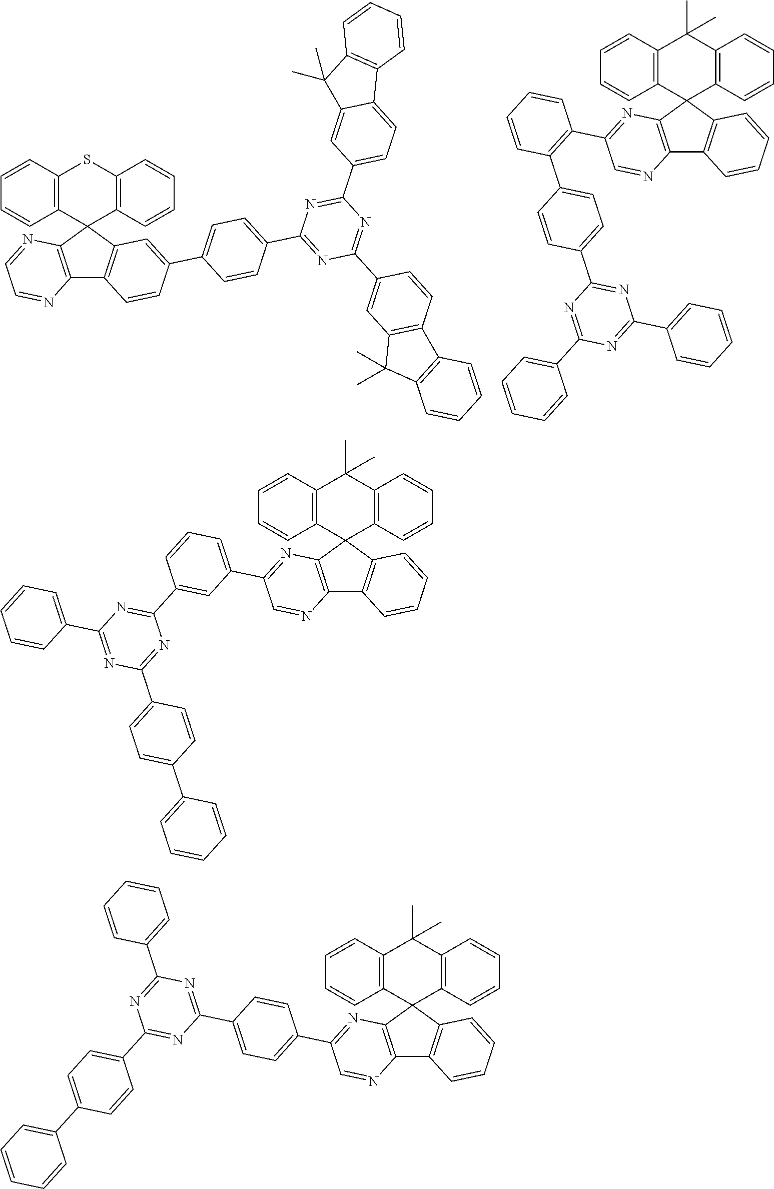

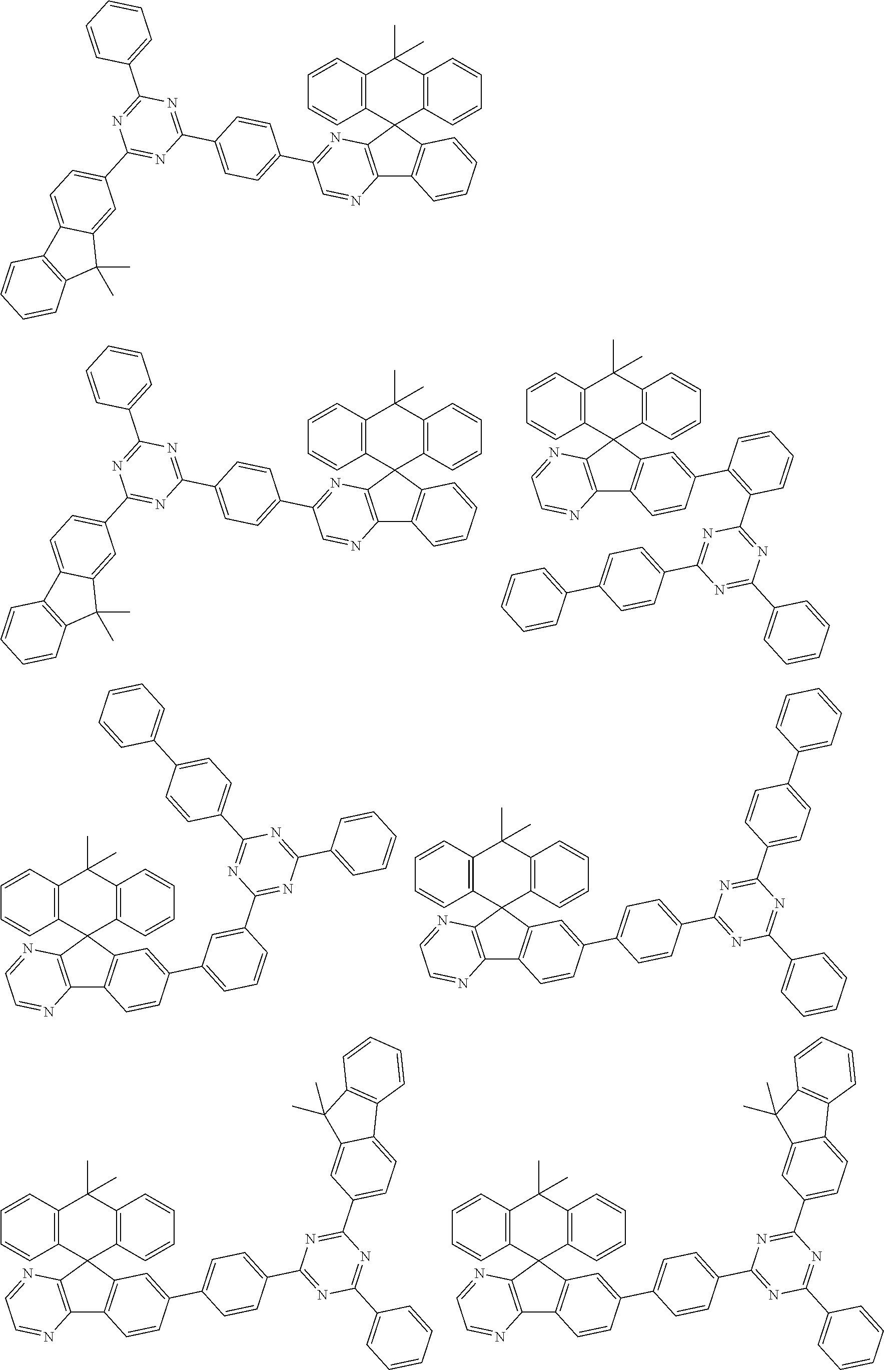

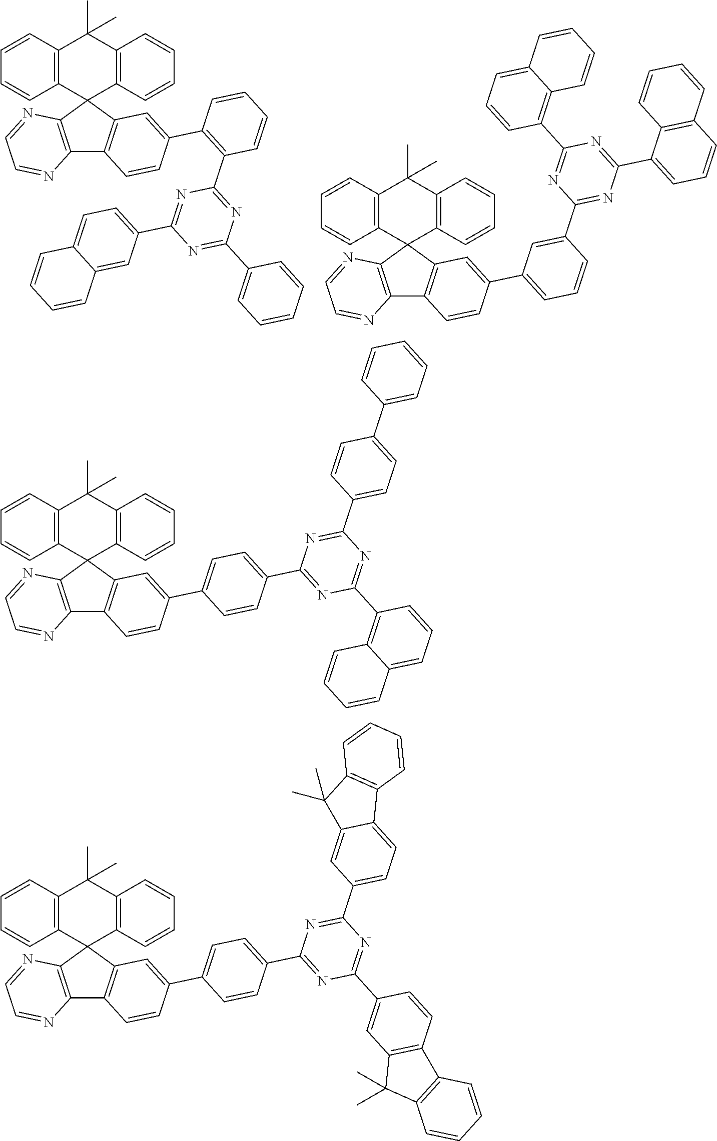

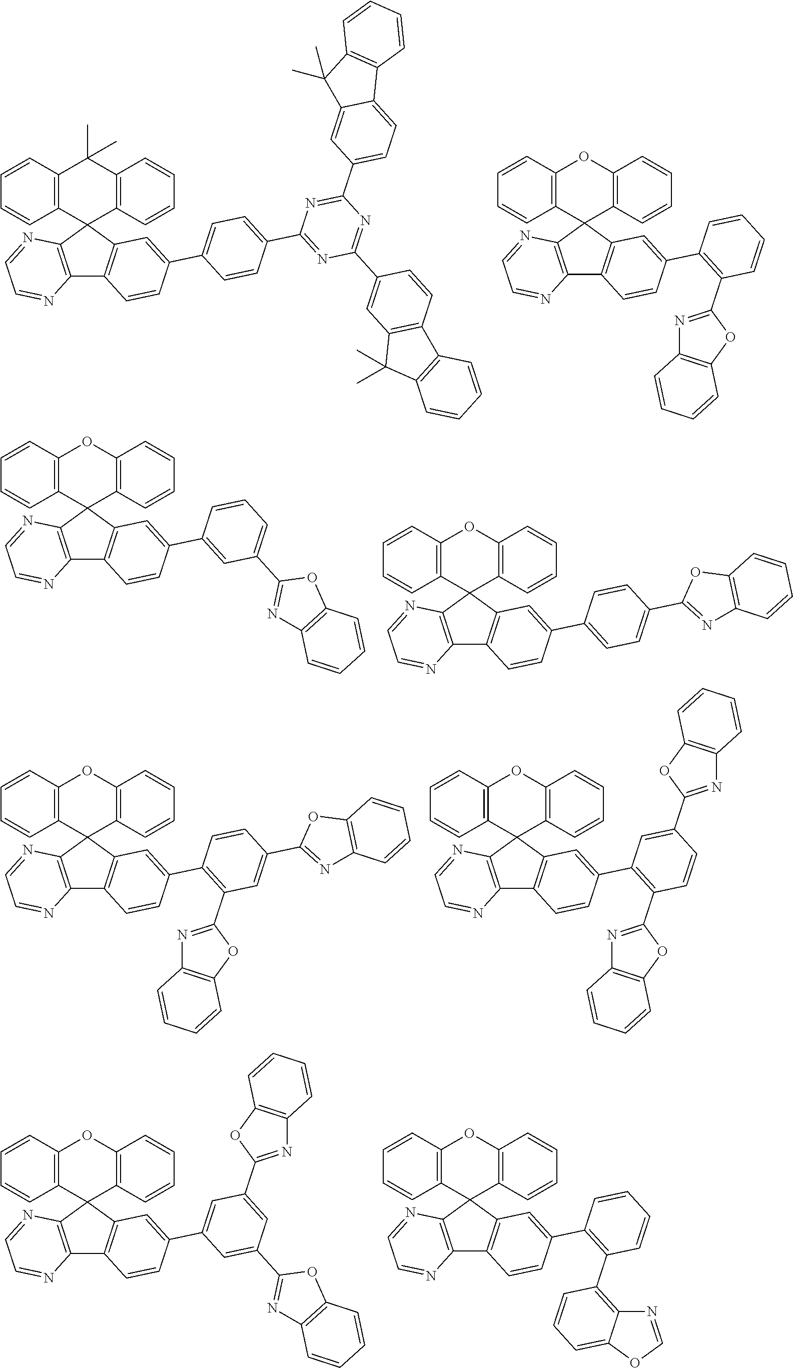

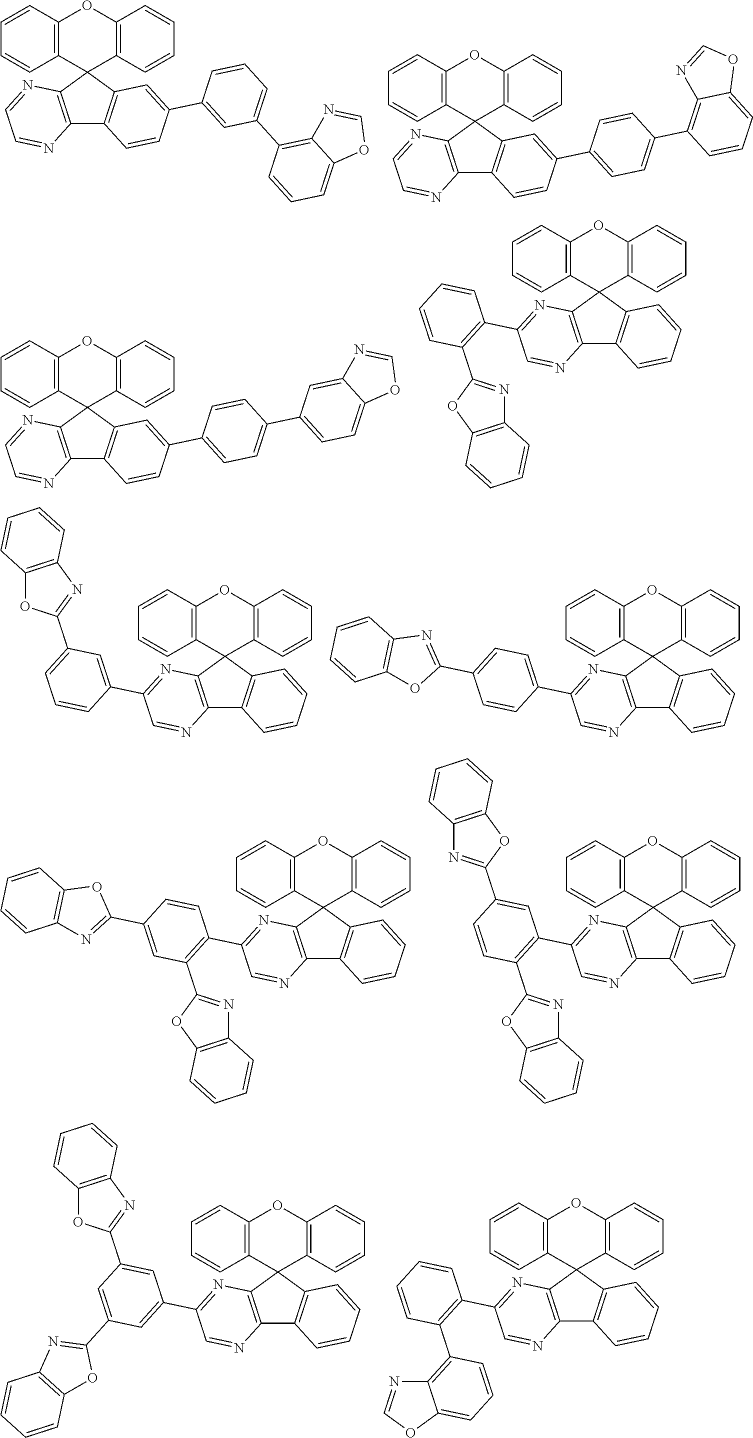

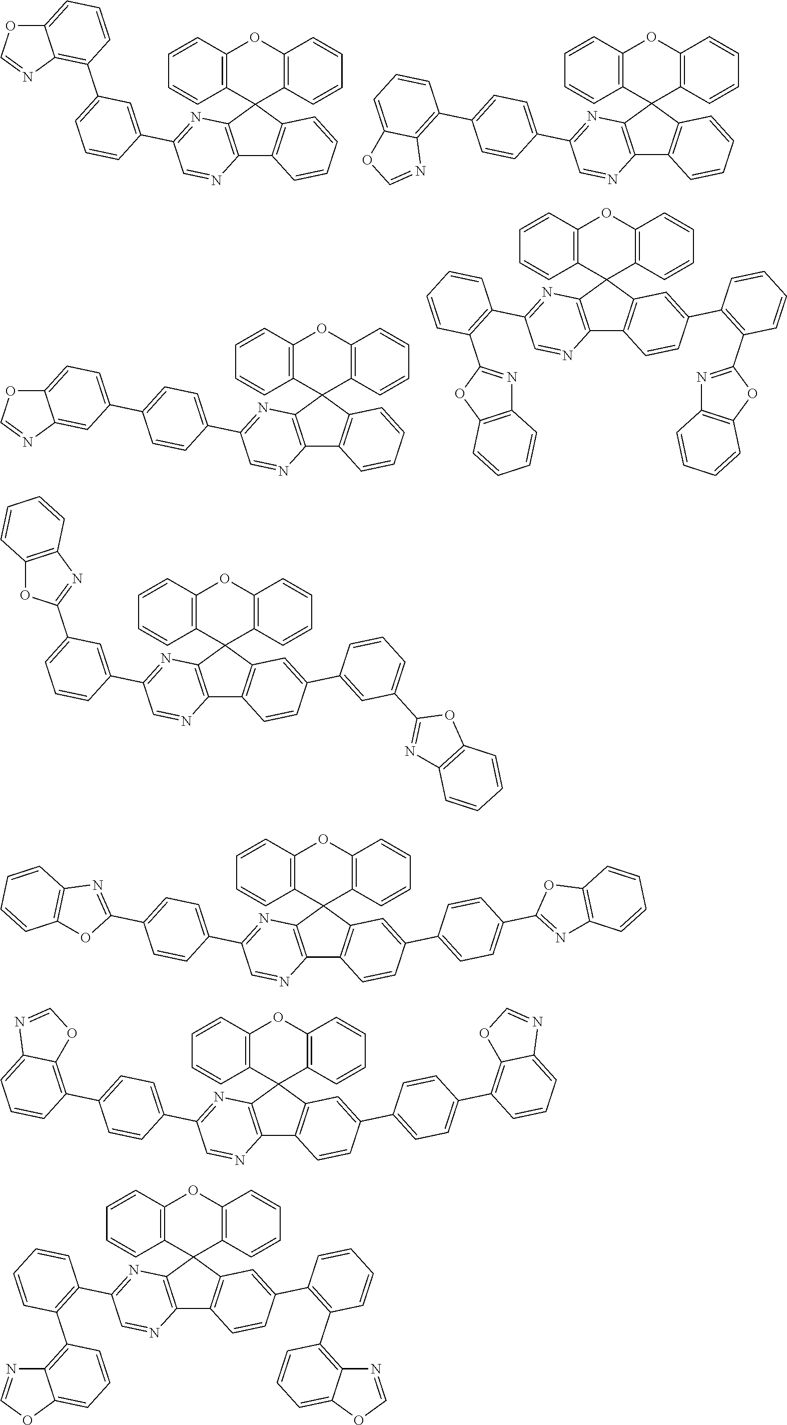

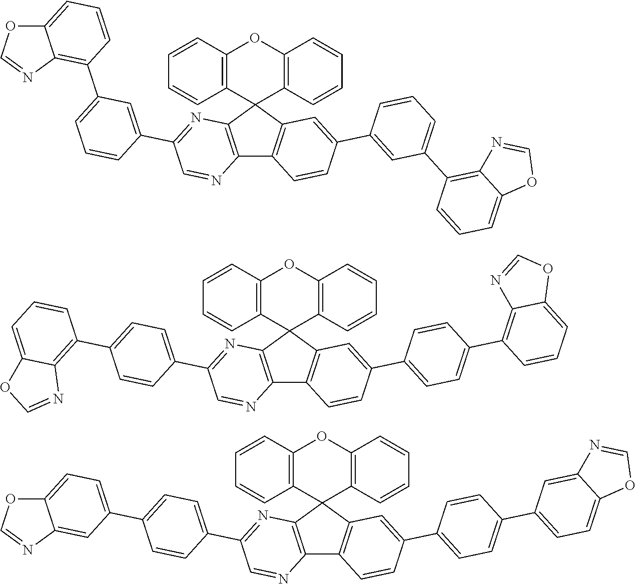

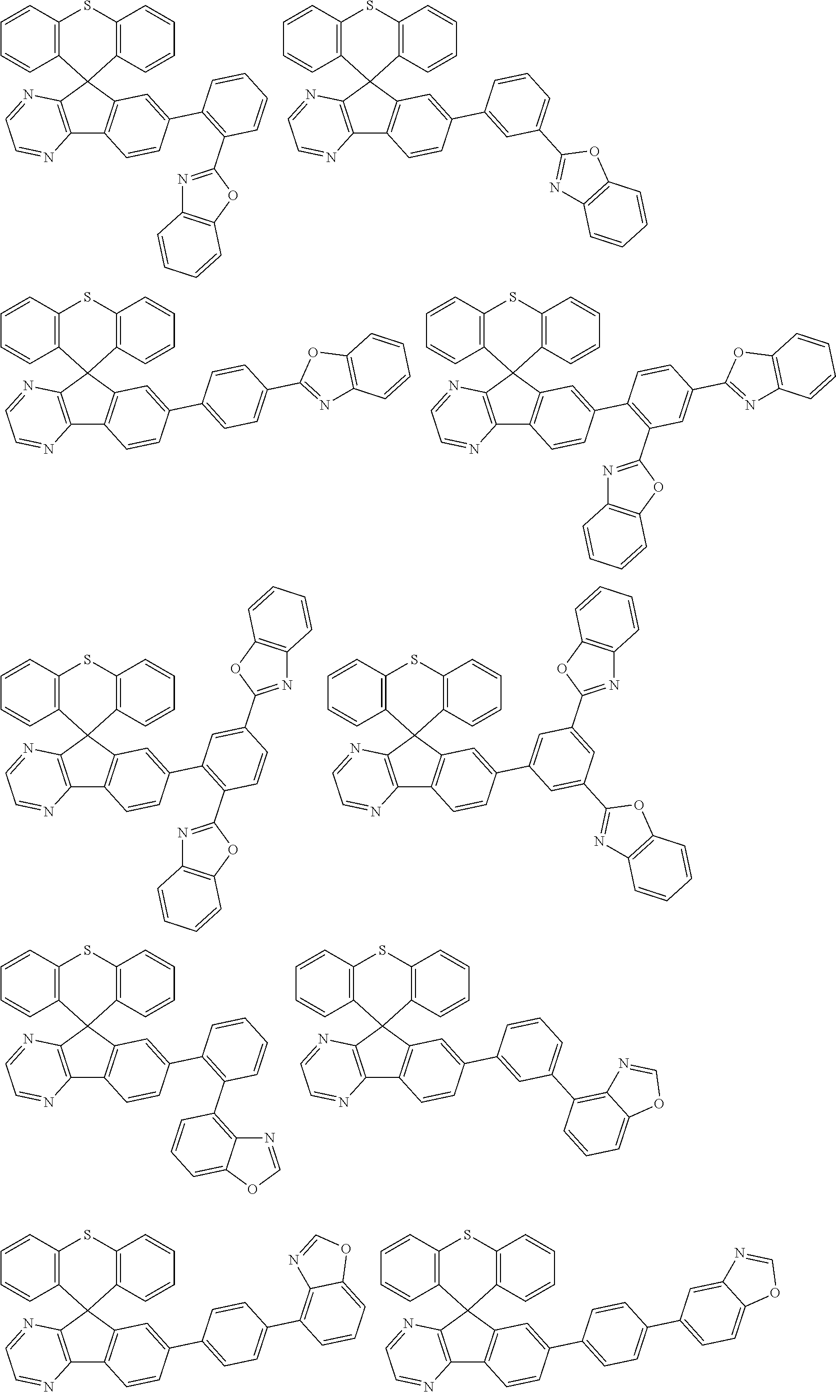

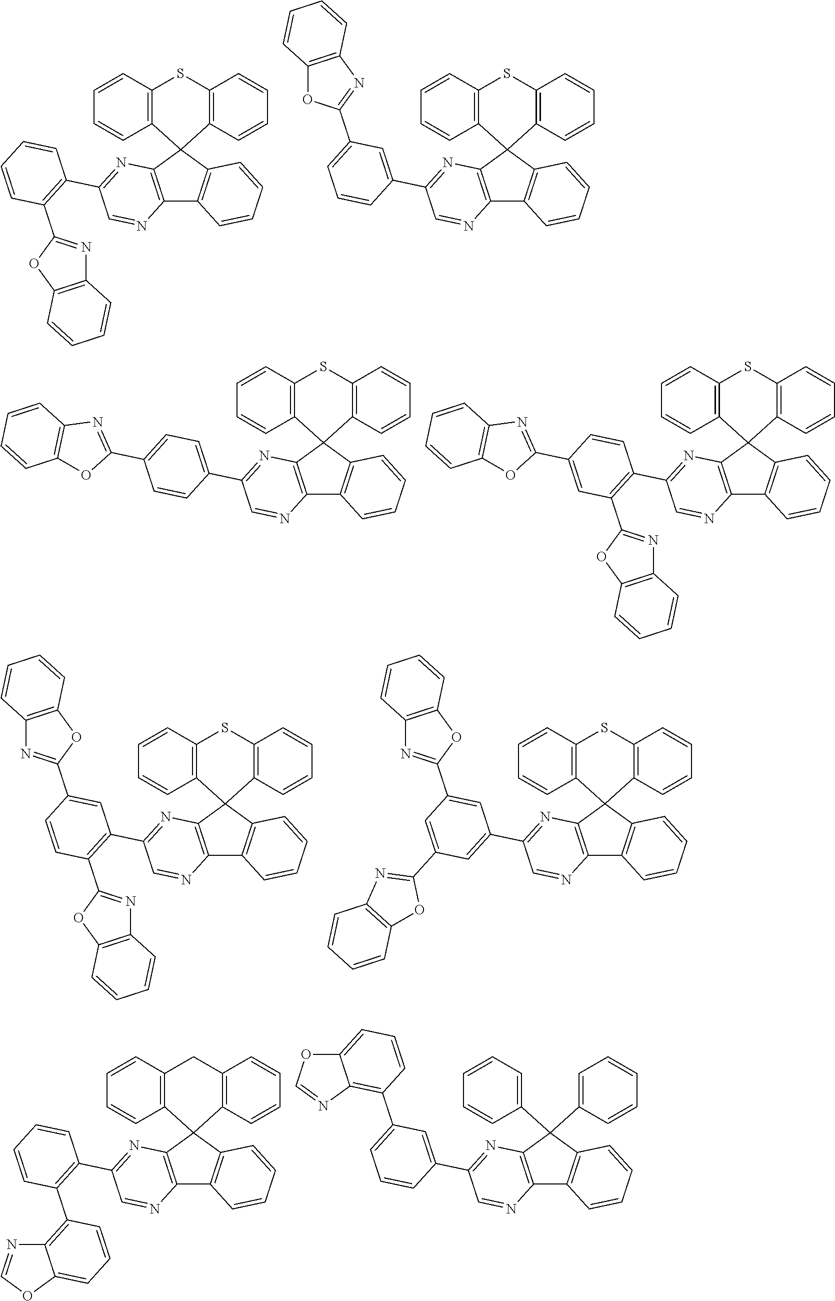

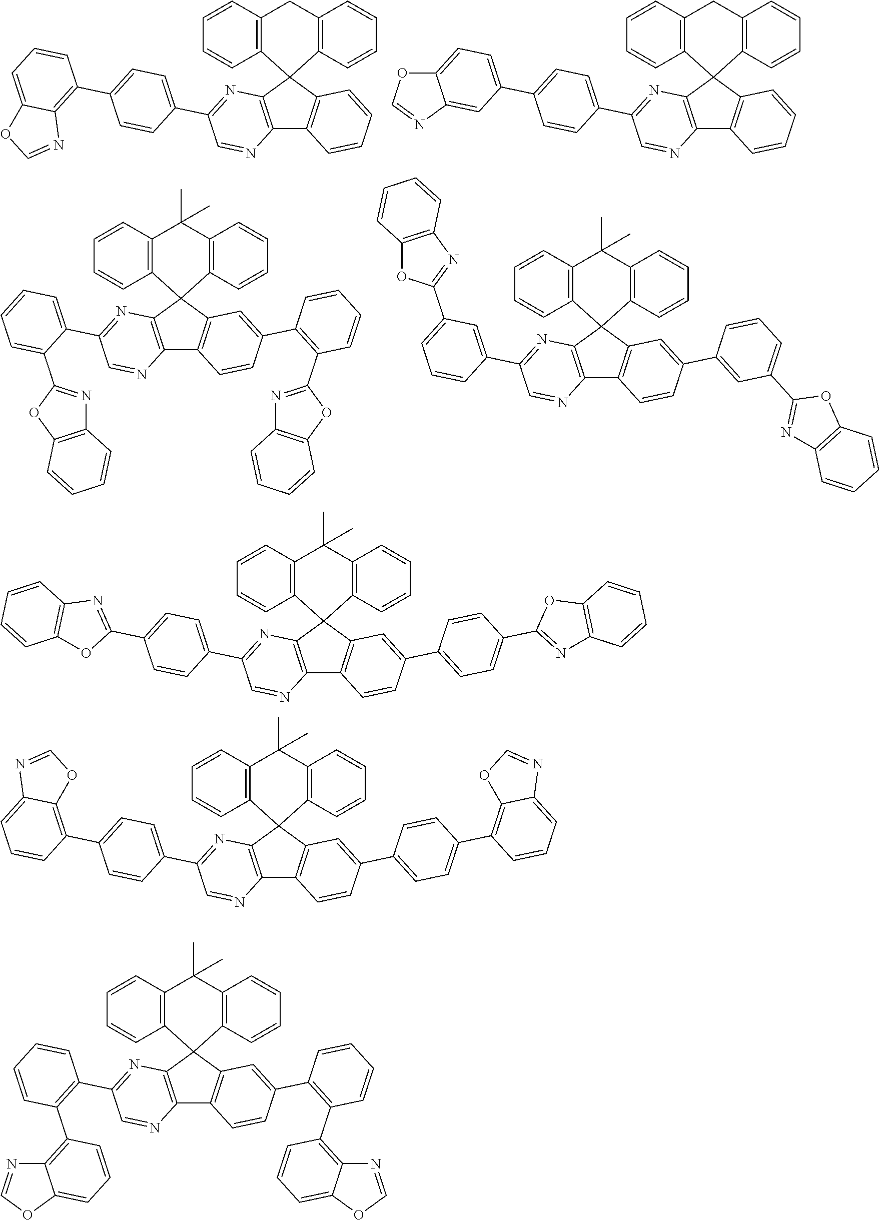

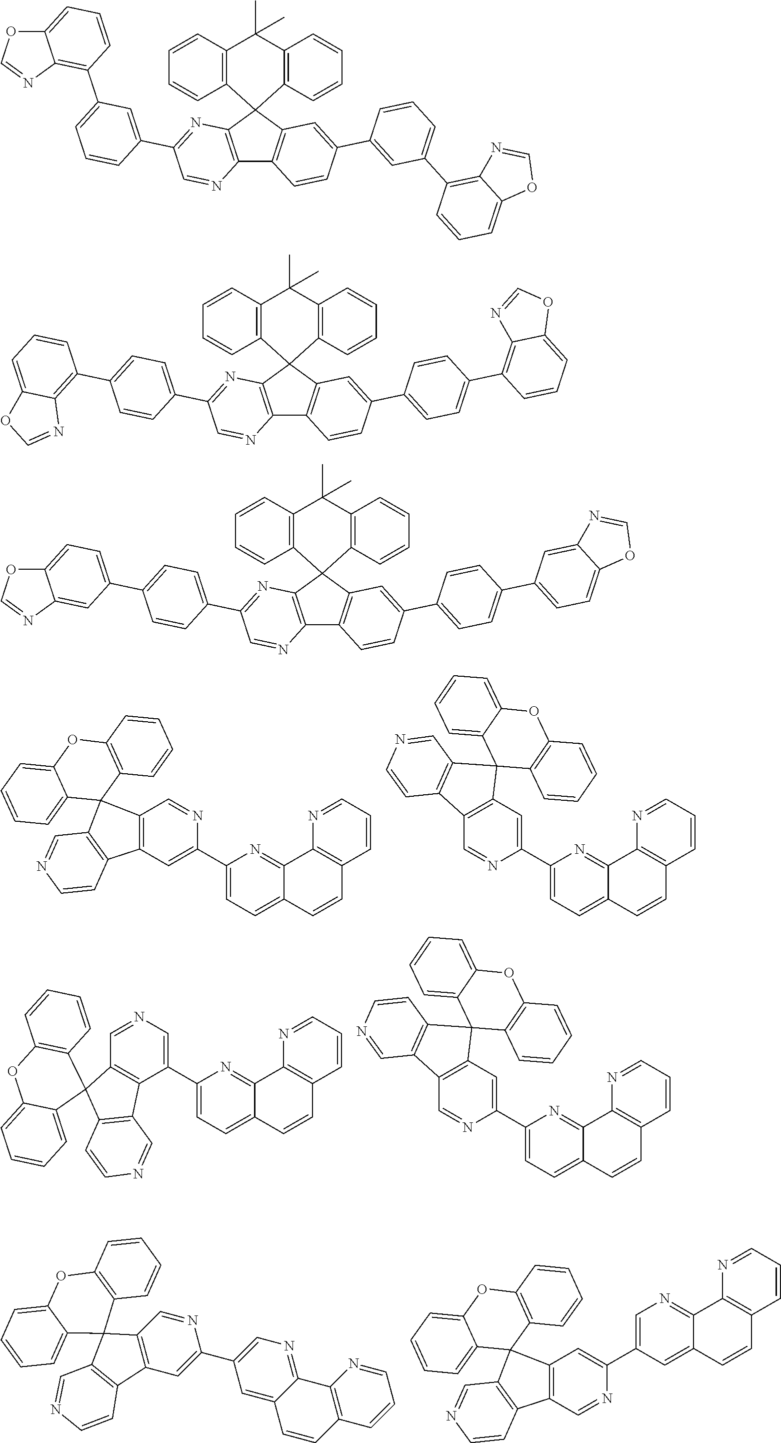

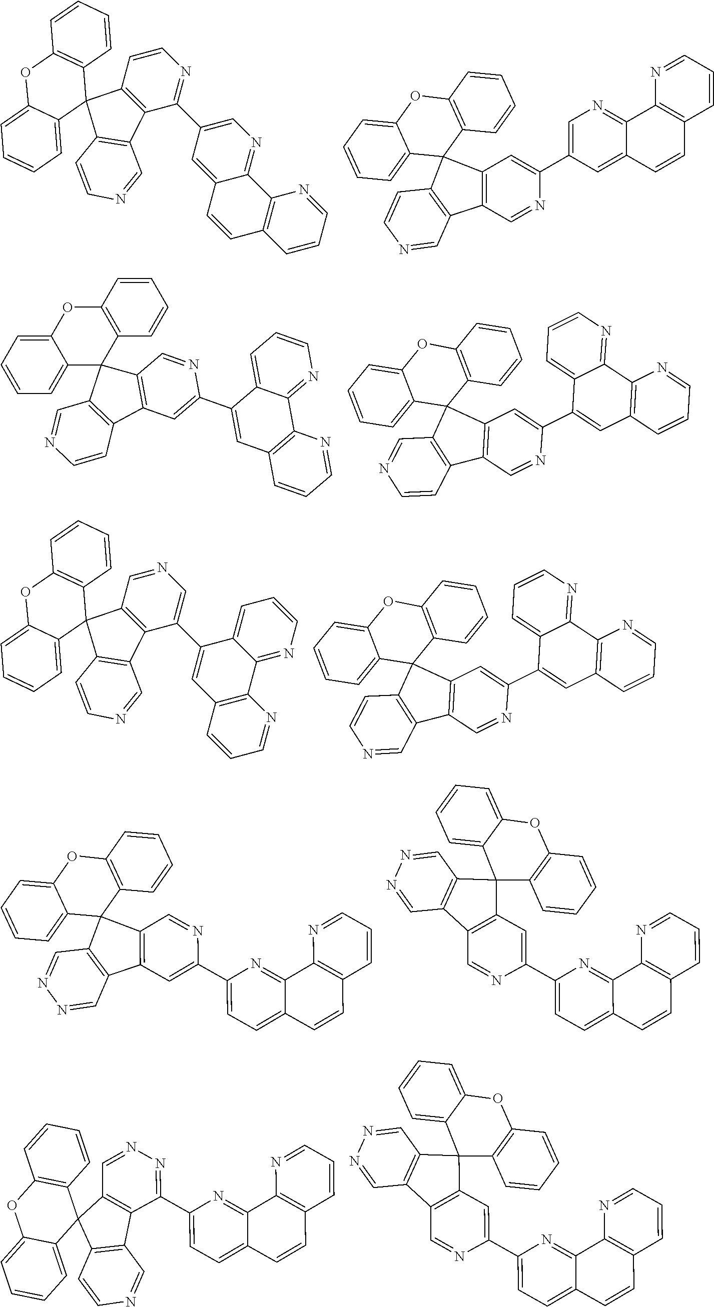

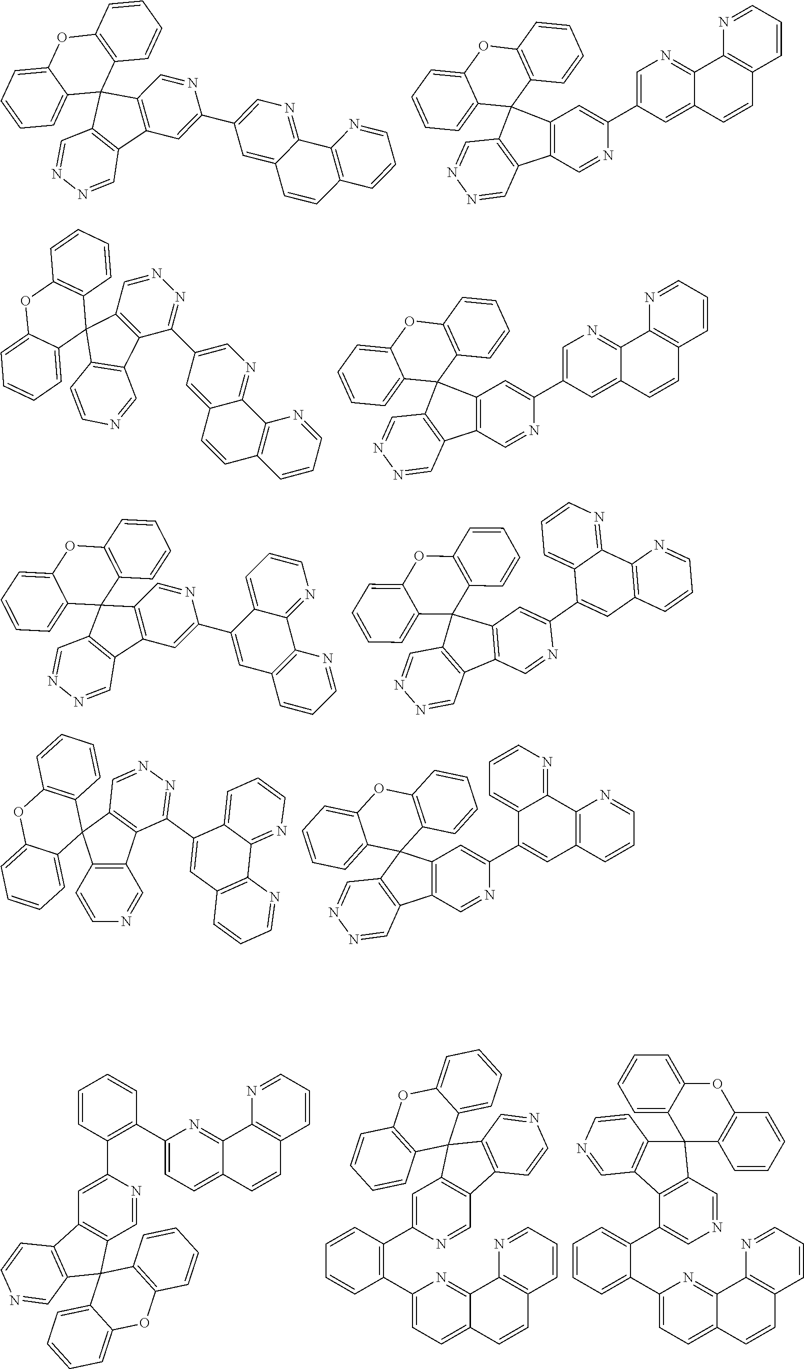

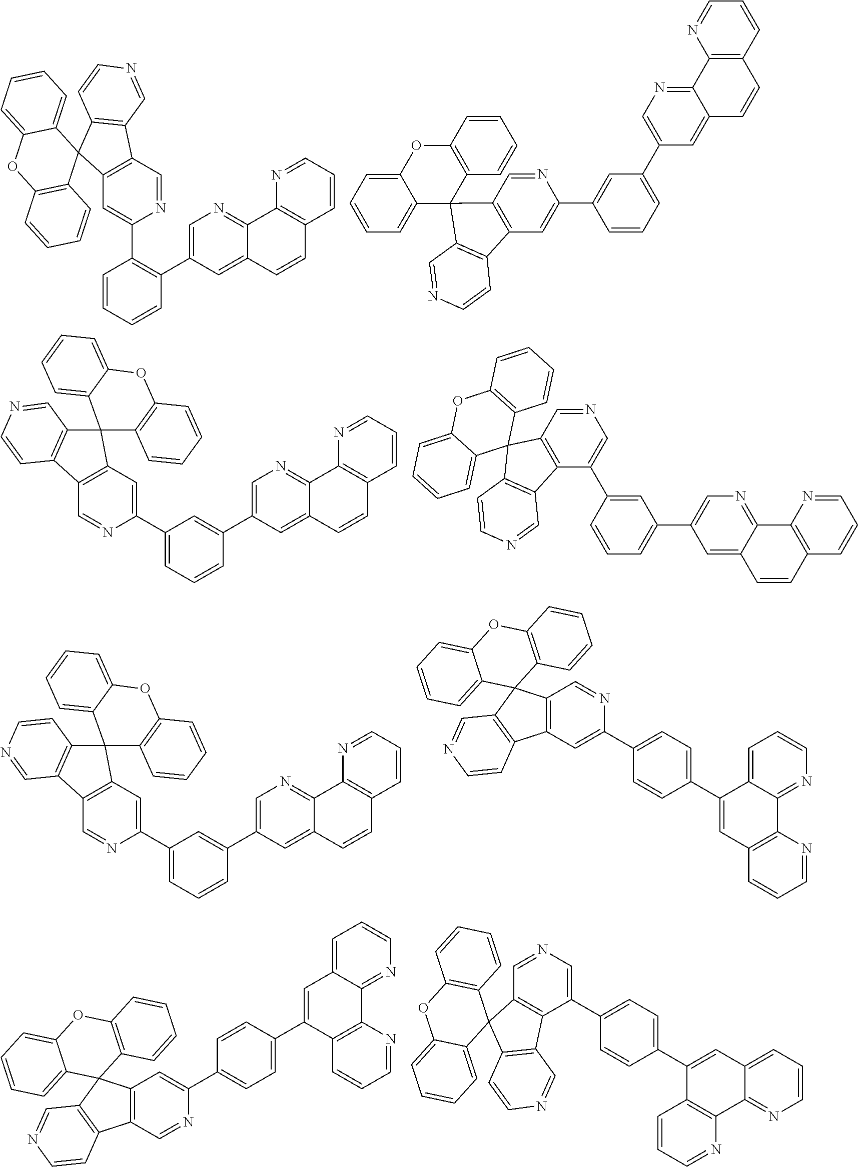

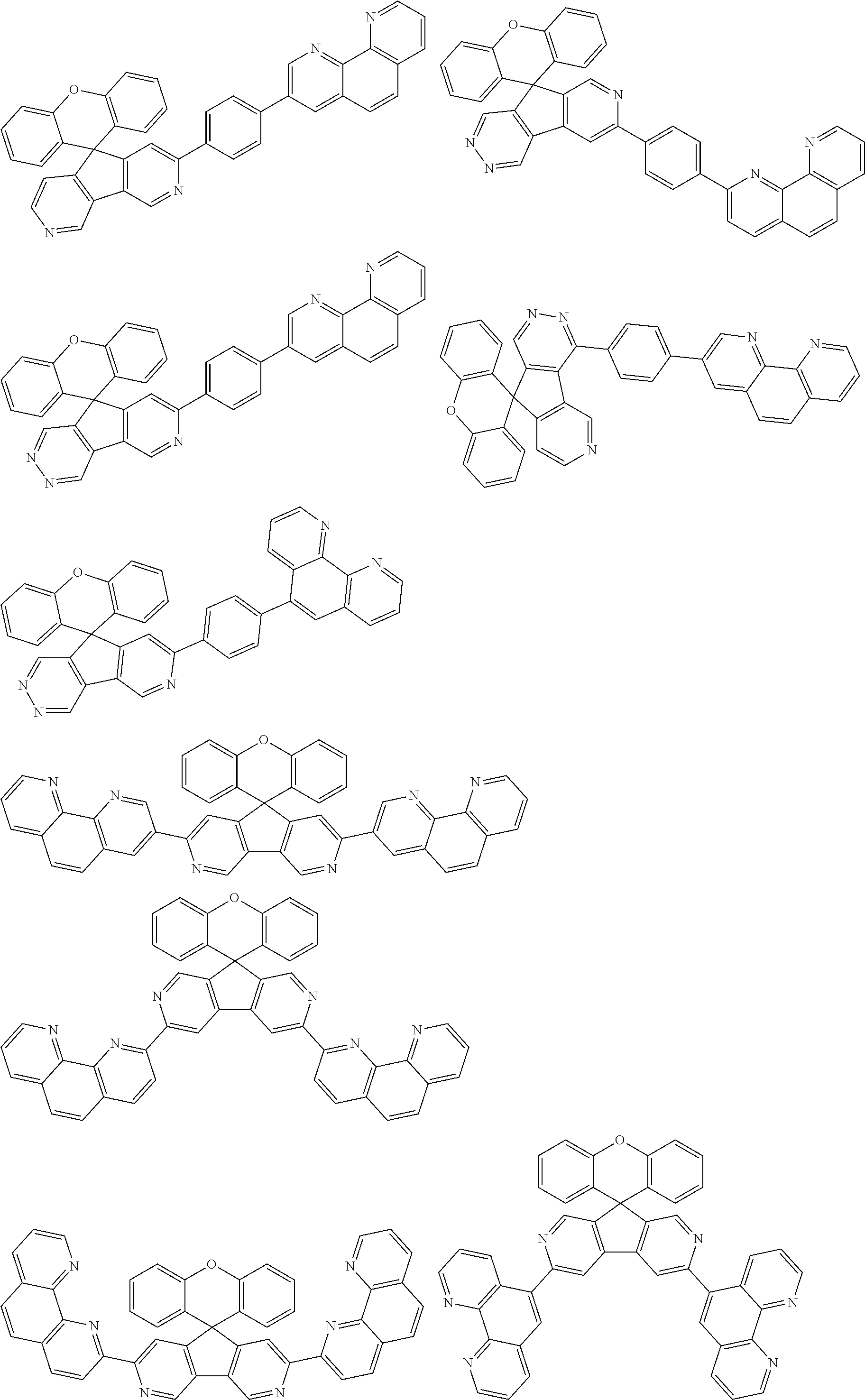

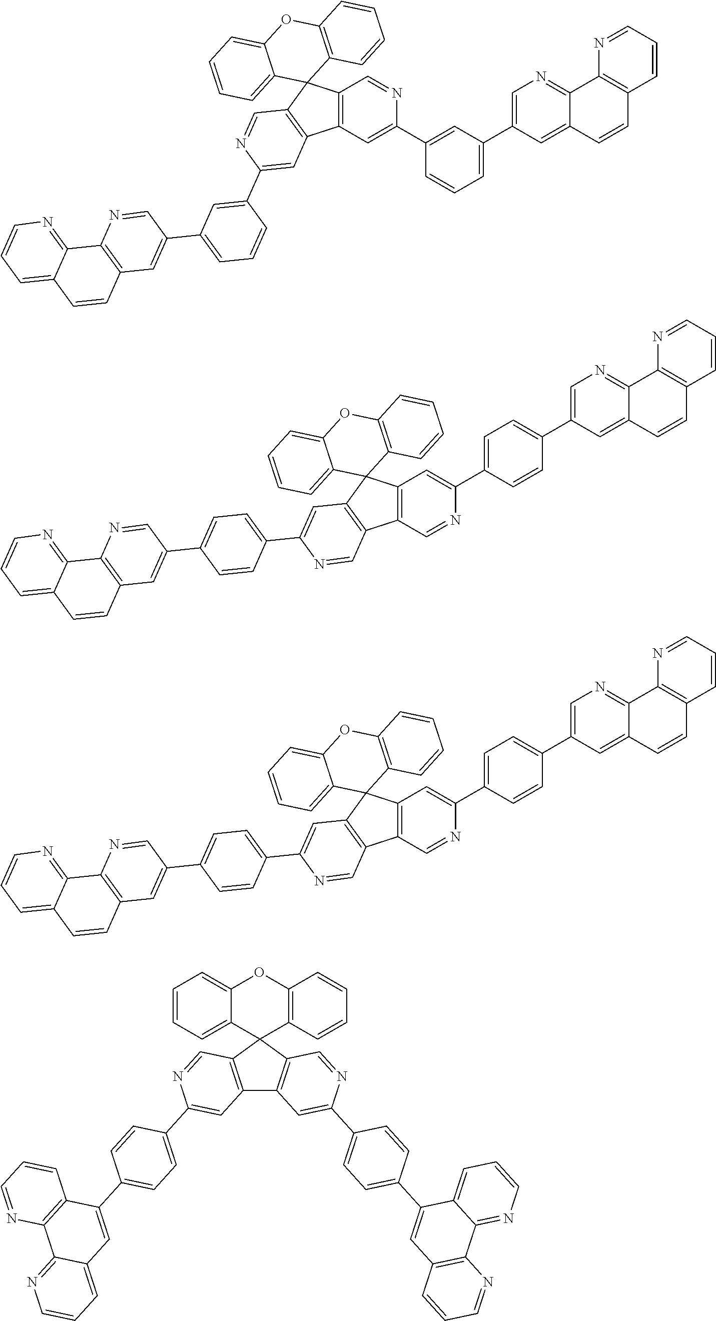 [SMILES: c1cc(-c2cnc3c(ccc4cccnc43)c2)cc(-c2cc3c(cn2)C2(c4ccccc4Oc4ccccc42)c2cnc(-c4cccc(-c5cnc6c(ccc7cccnc76)c5)c4)cc2-3)c1 ; c1ccc2c(c1)Oc1ccccc1C21c2cc(-c3ccc(-c4cnc5c(ccc6cccnc65)c4)cc3)ncc2-c2cnc(-c3ccc(-c4cnc5c(ccc6cccnc65)c4)cc3)cc21 ; c1ccc2c(c1)Oc1ccccc1C21c2cc(-c3ccc(-c4cnc5c(ccc6cccnc65)c4)cc3)ncc2-c2cnc(-c3ccc(-c4cnc5c(ccc6cccnc65)c4)cc3)cc21 ; c1ccc2c(c1)Oc1ccccc1C21c2cnc(-c3ccc(-c4cc5cccnc5c5ncccc45)cc3)cc2-c2cc(-c3ccc(-c4cc5cccnc5c5ncccc45)cc3)ncc21]

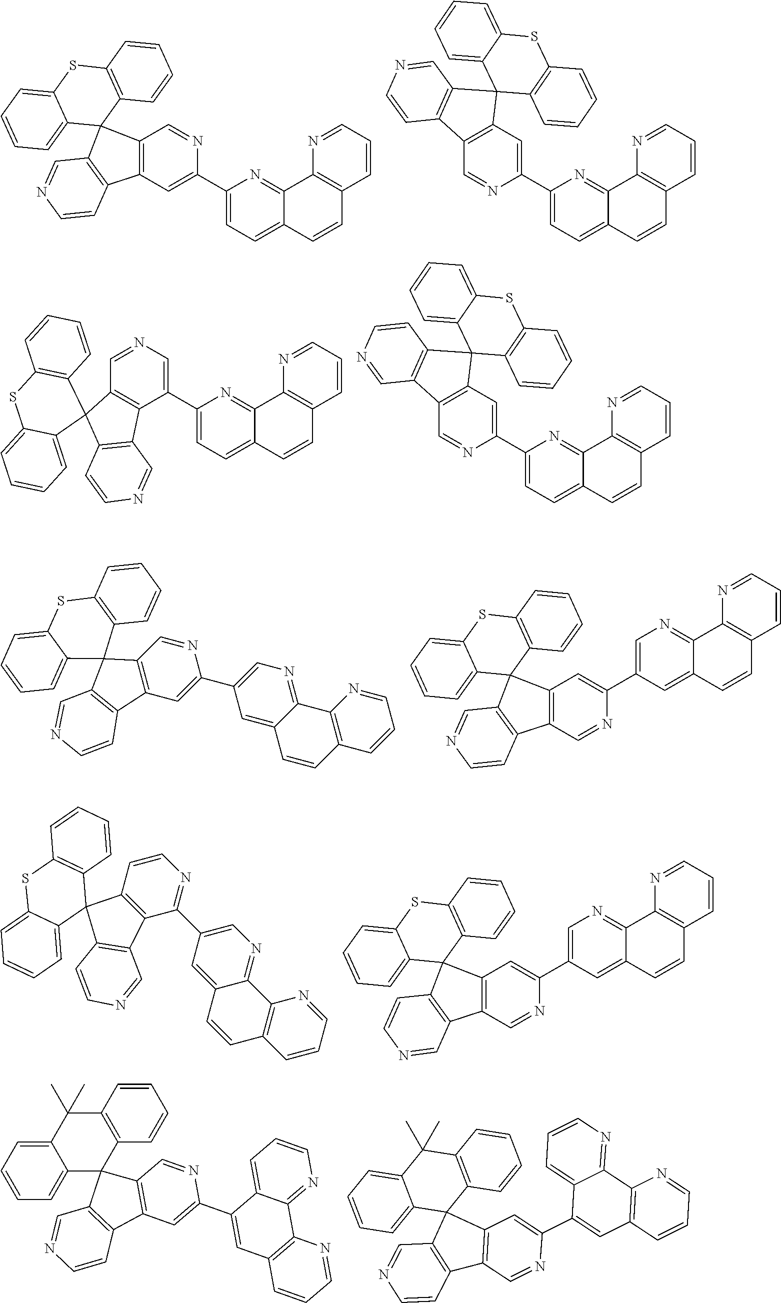

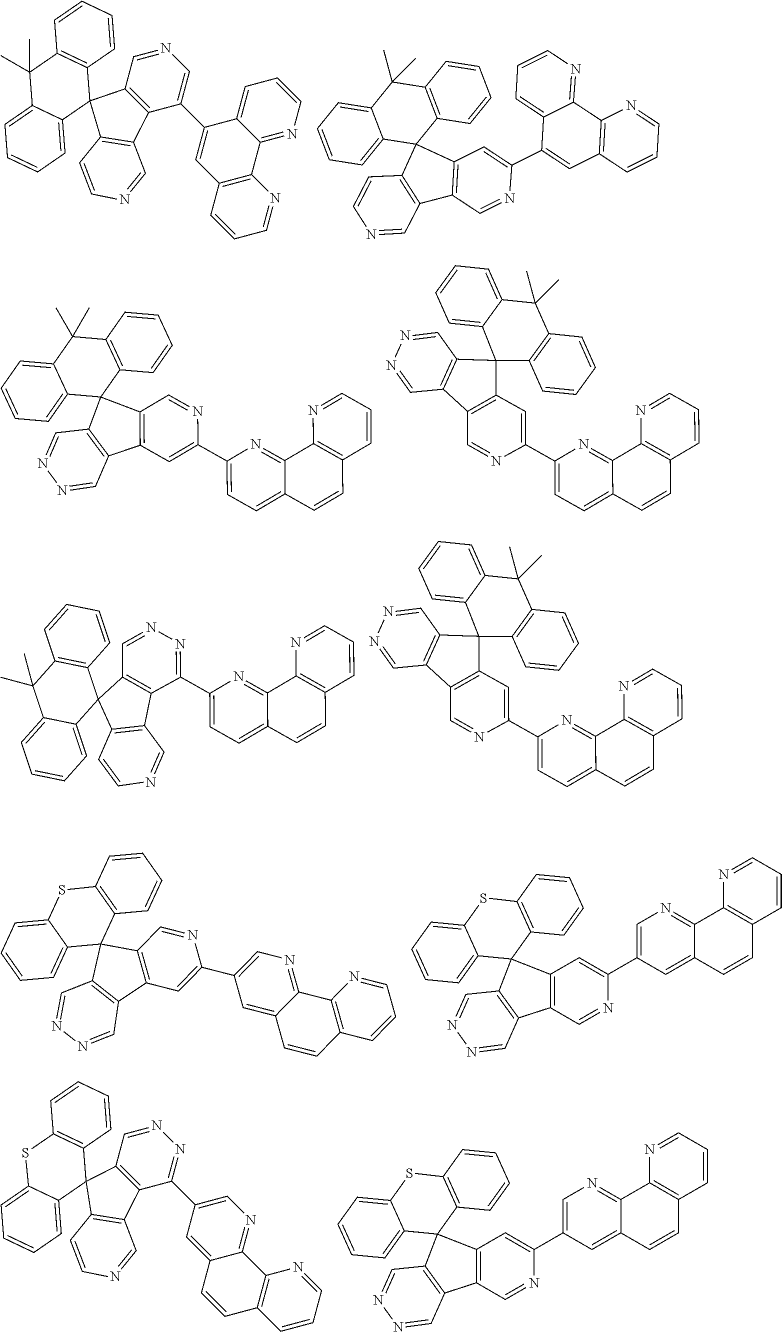

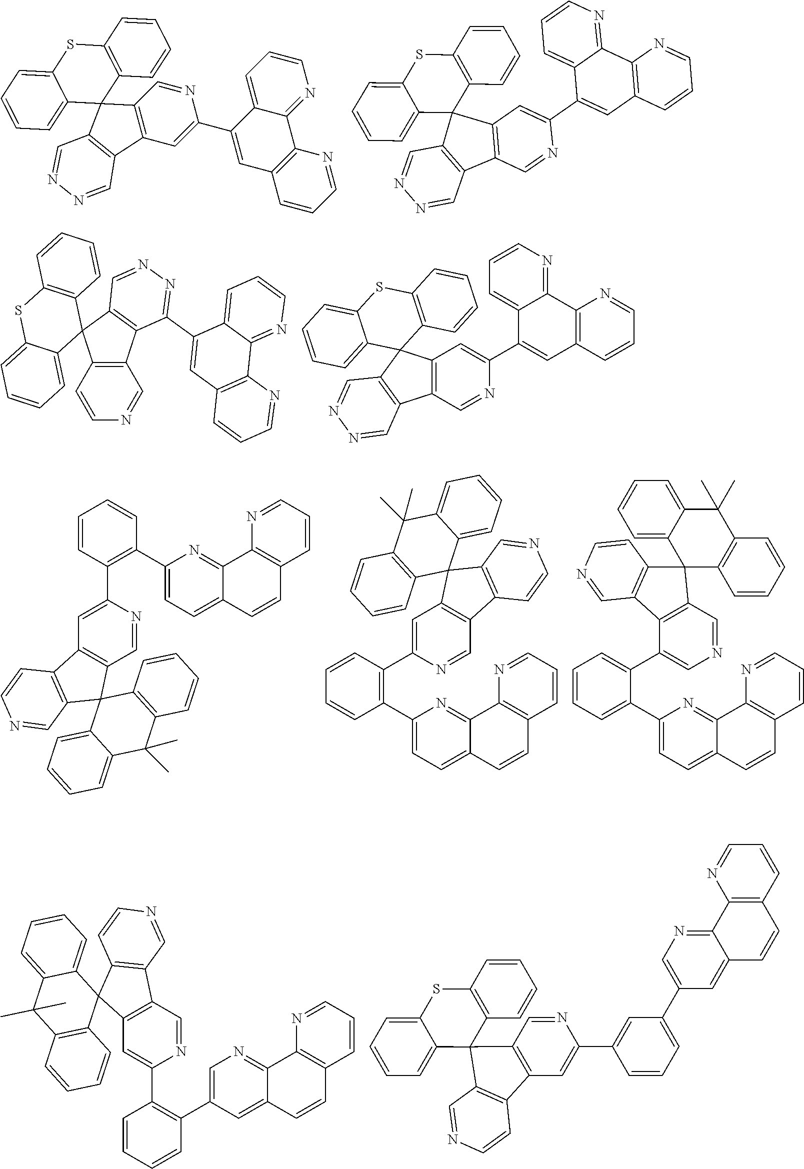

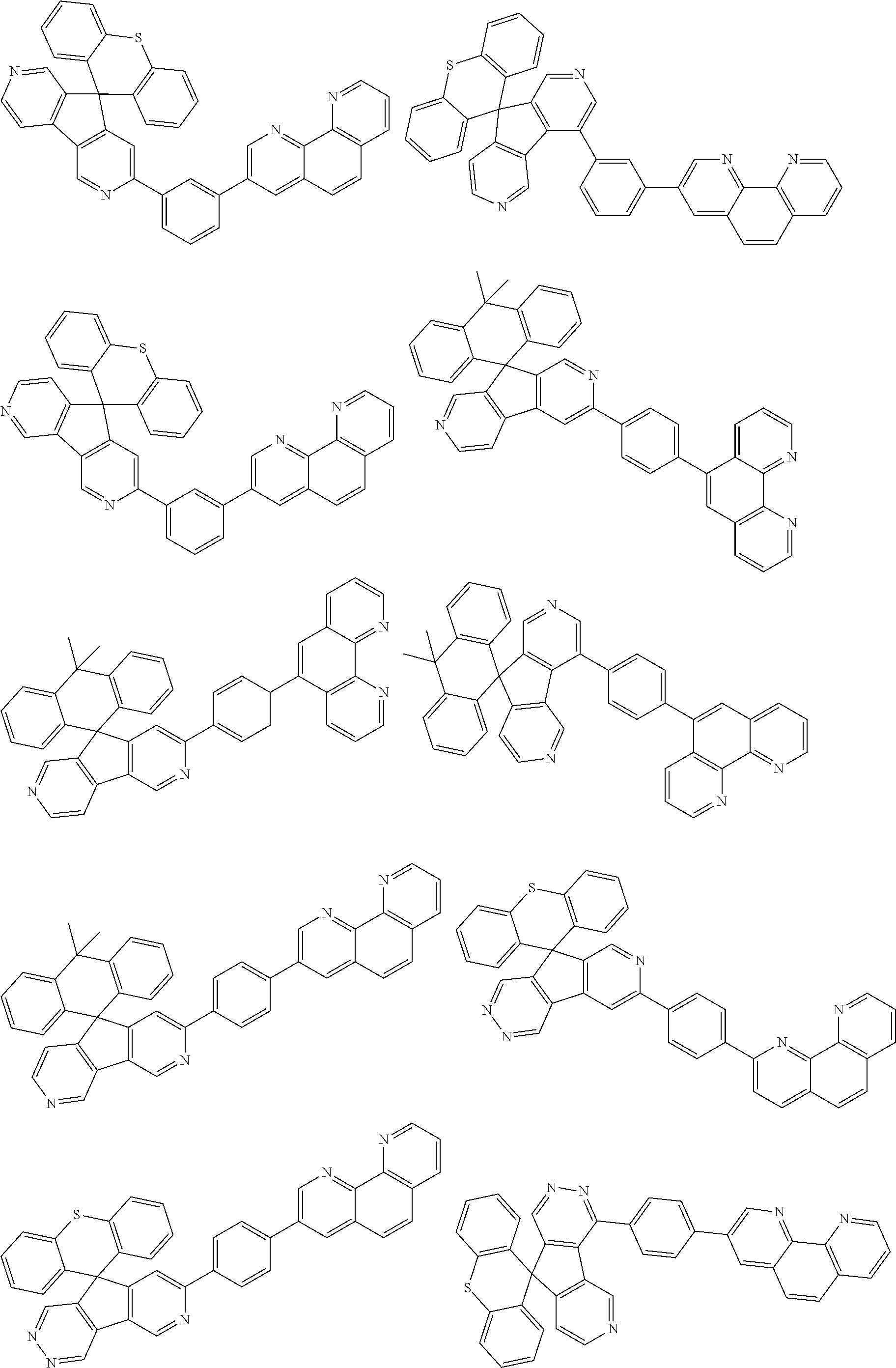

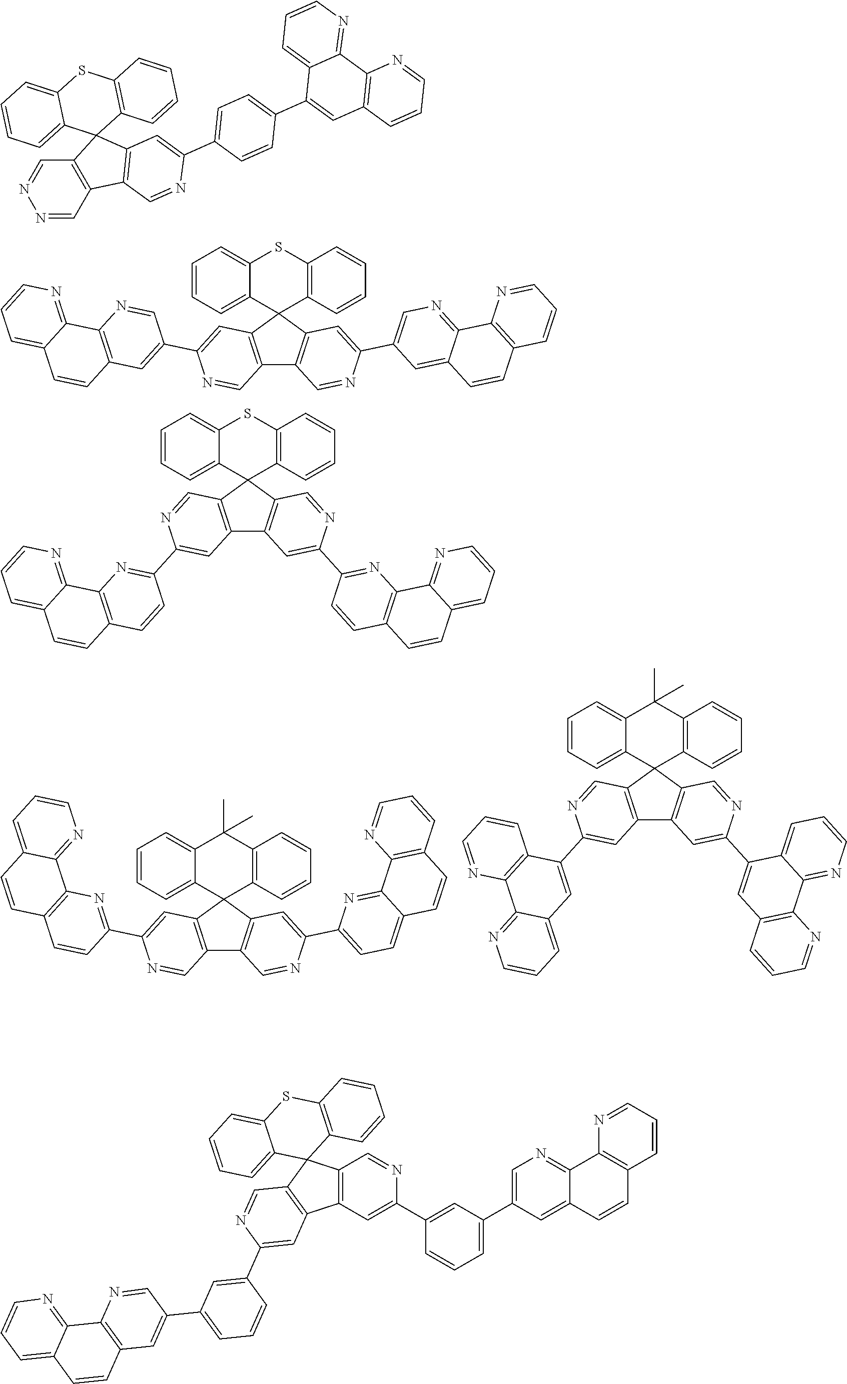

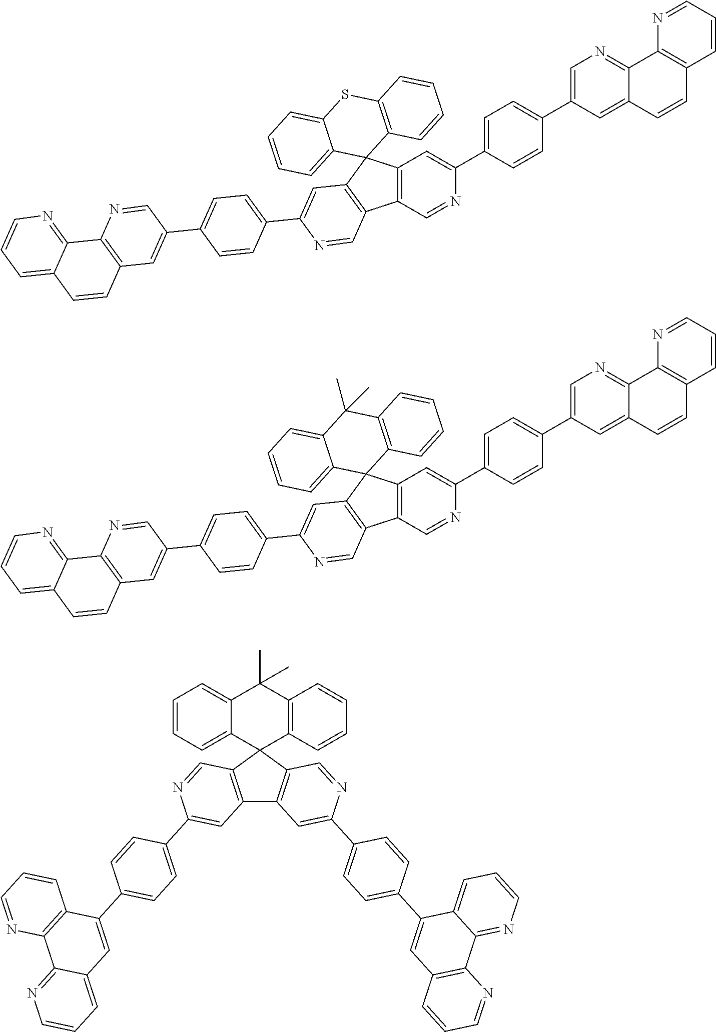 [SMILES: c1ccc2c(c1)Sc1ccccc1C21c2cc(-c3ccc(-c4cnc5c(ccc6cccnc65)c4)cc3)ncc2-c2cnc(-c3ccc(-c4cnc5c(ccc6cccnc65)c4)cc3)cc21 ; CC1(C)c2ccccc2C2(c3cc(-c4ccc(-c5cnc6c(ccc7cccnc76)c5)cc4)ncc3-c3cnc(-c4ccc(-c5cnc6c(ccc7cccnc76)c5)cc4)cc32)c2ccccc21 ; CC1(C)c2ccccc2C2(c3cnc(-c4ccc(-c5cc6cccnc6c6ncccc56)cc4)cc3-c3cc(-c4ccc(-c5cc6cccnc6c6ncccc56)cc4)ncc32)c2ccccc21]

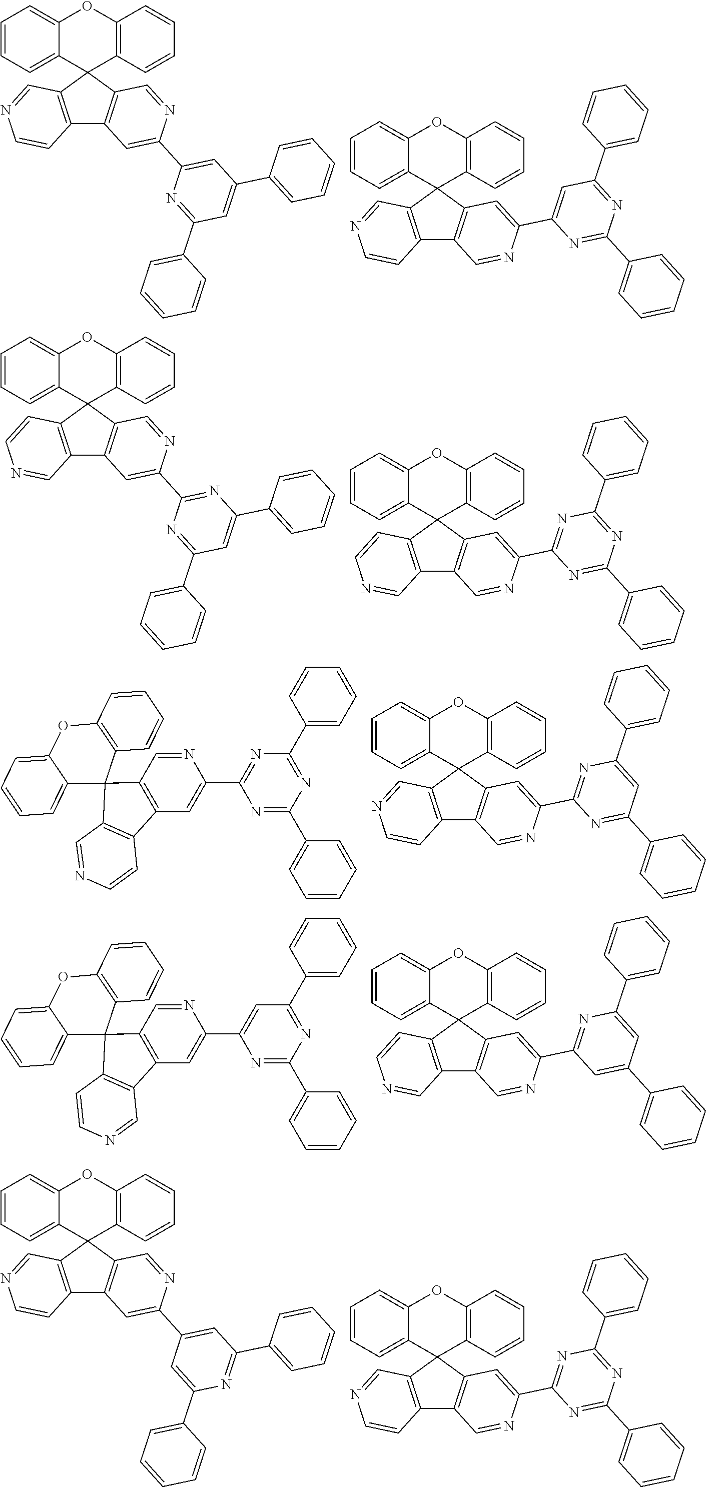

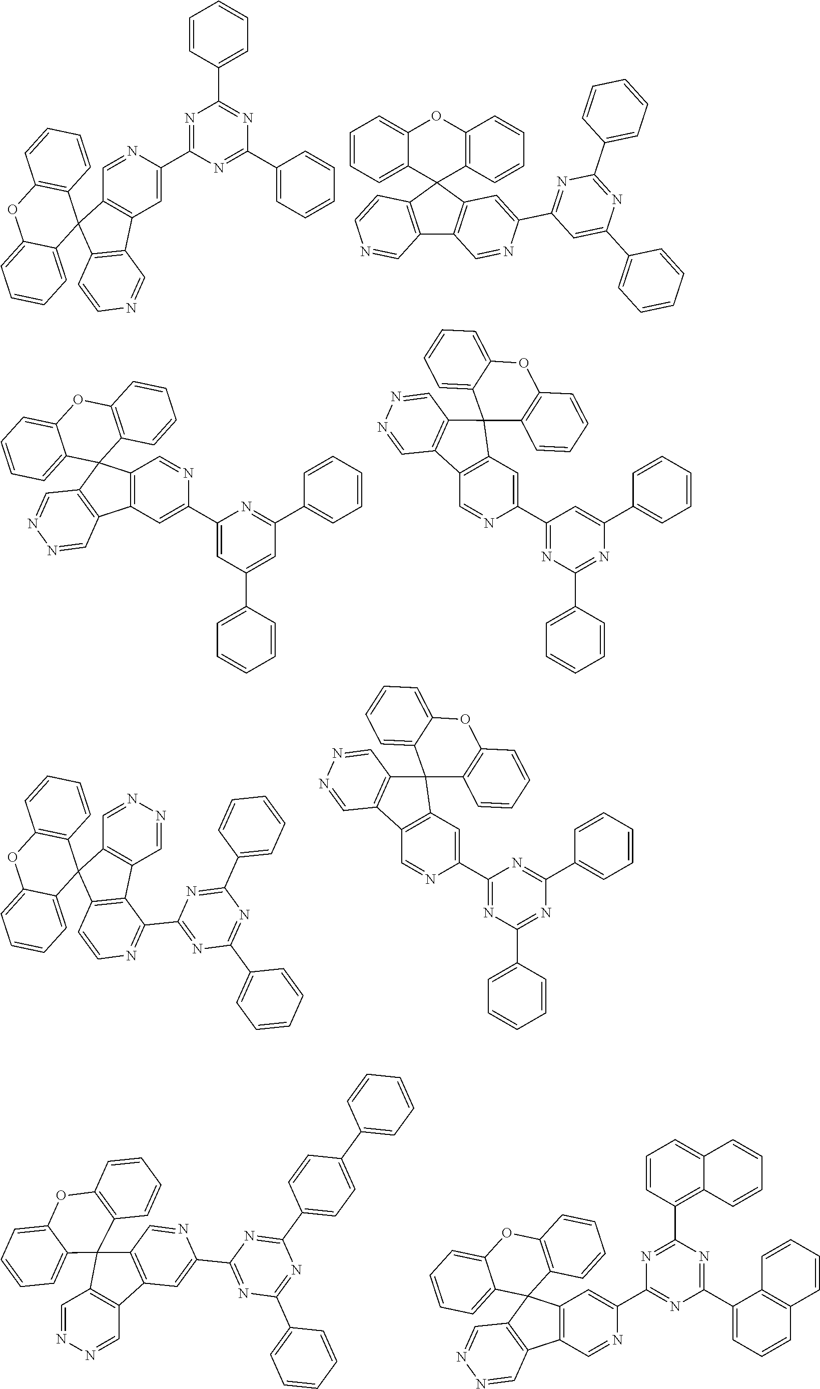

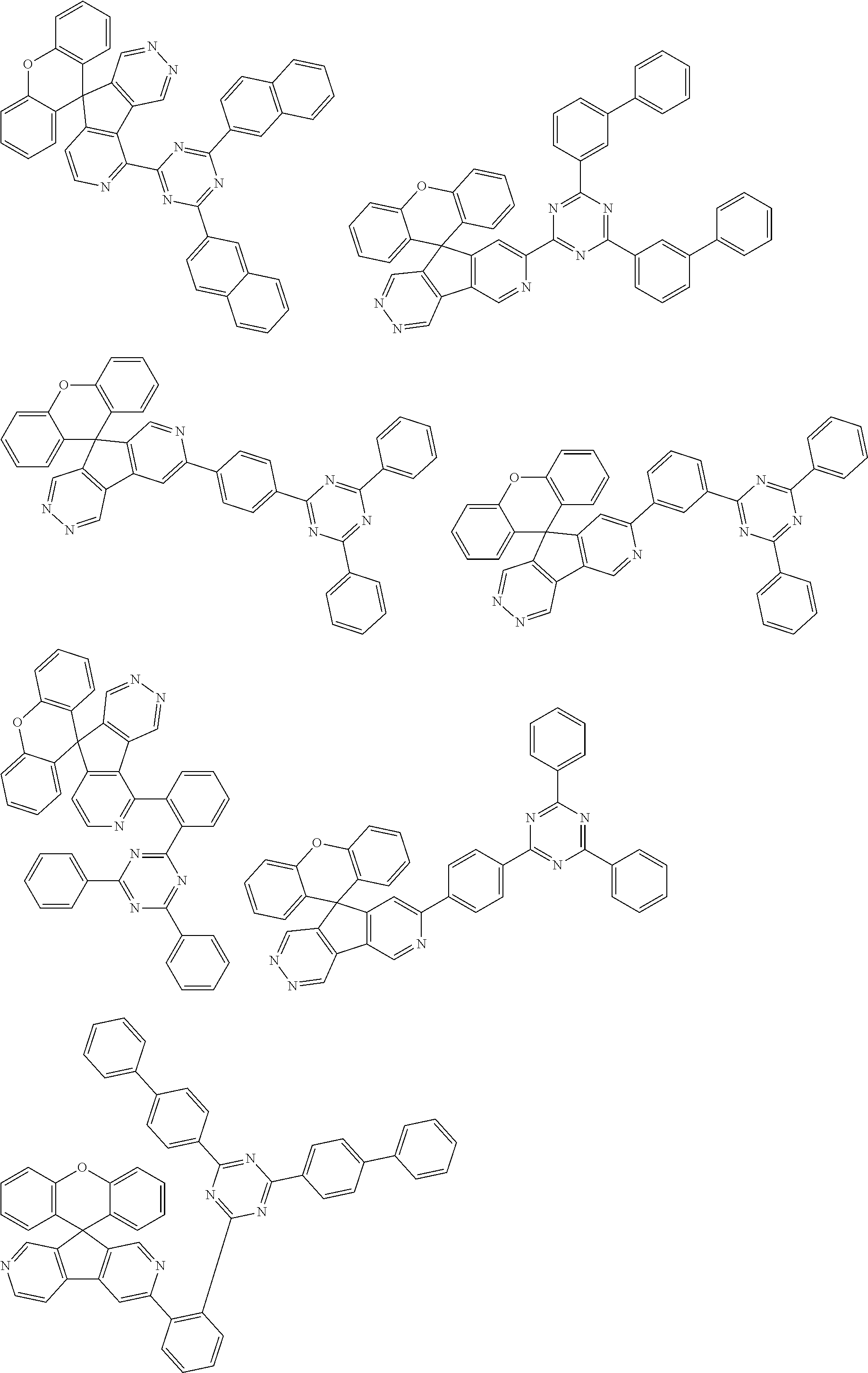

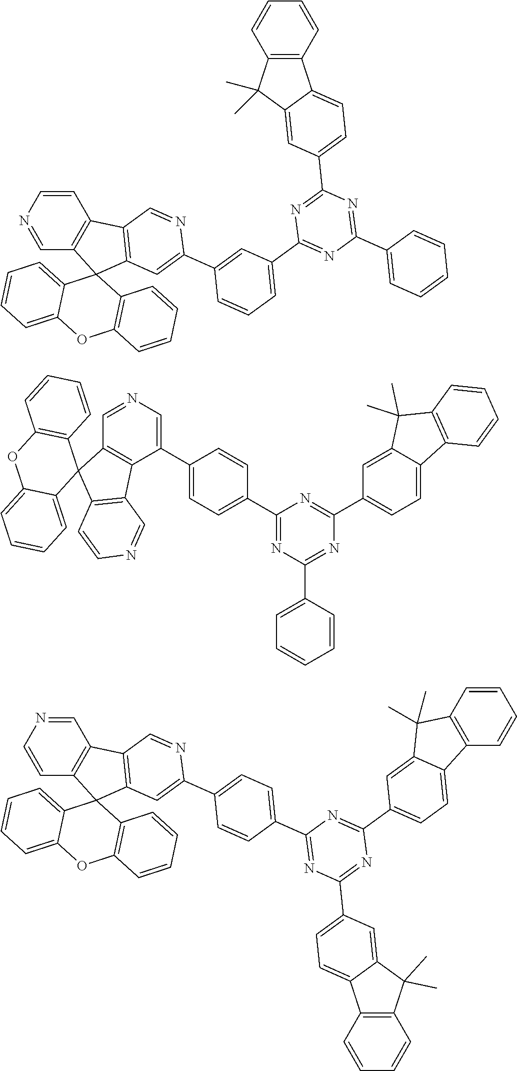

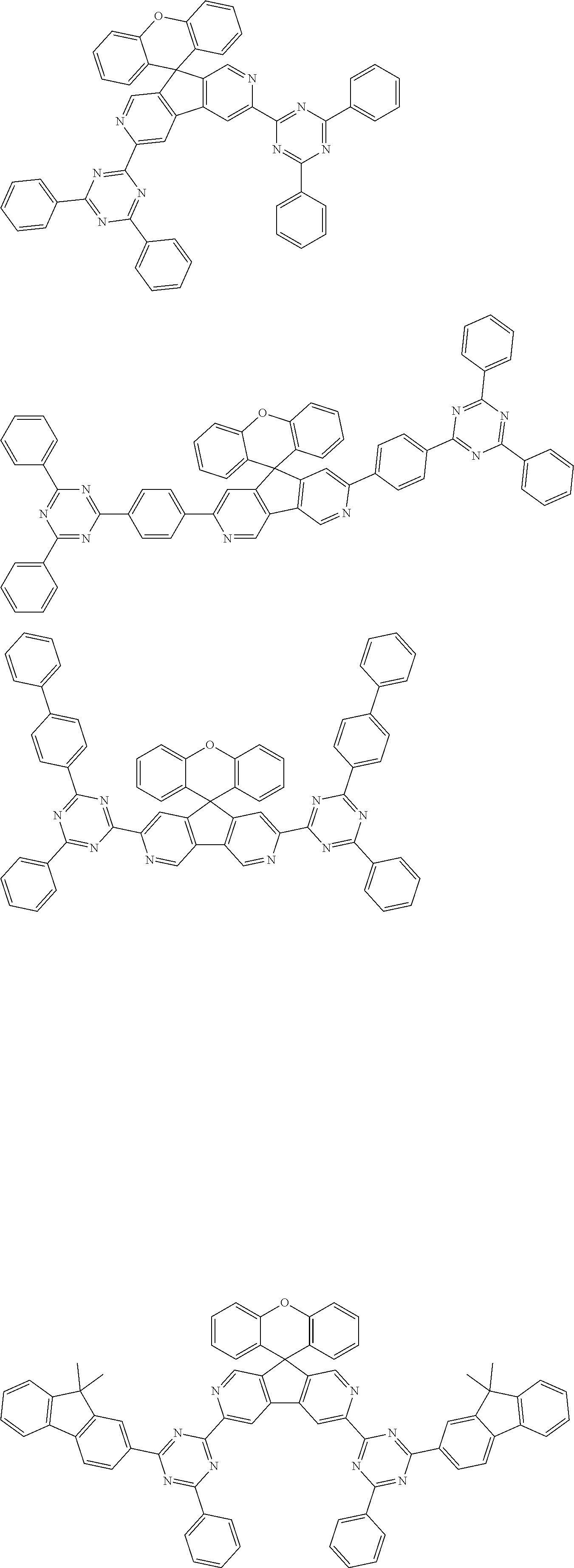

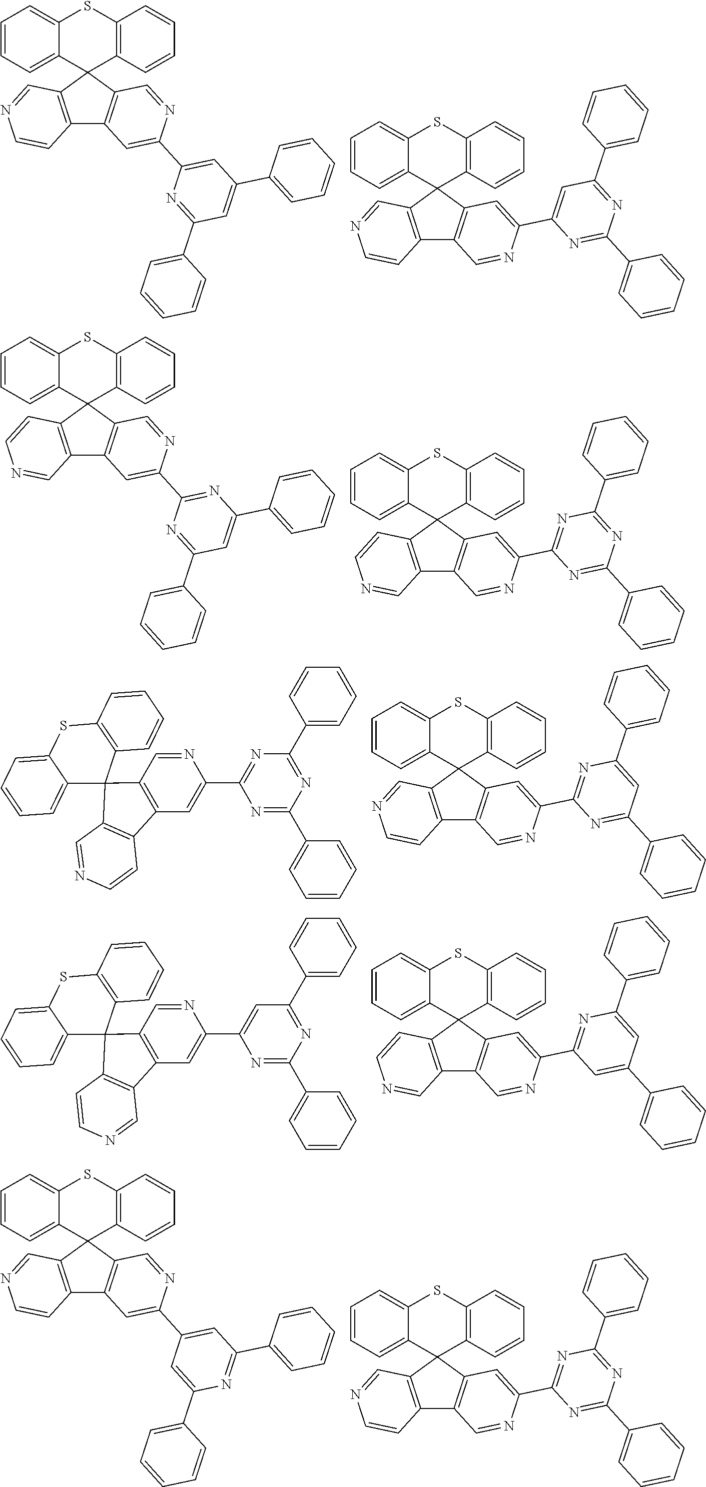

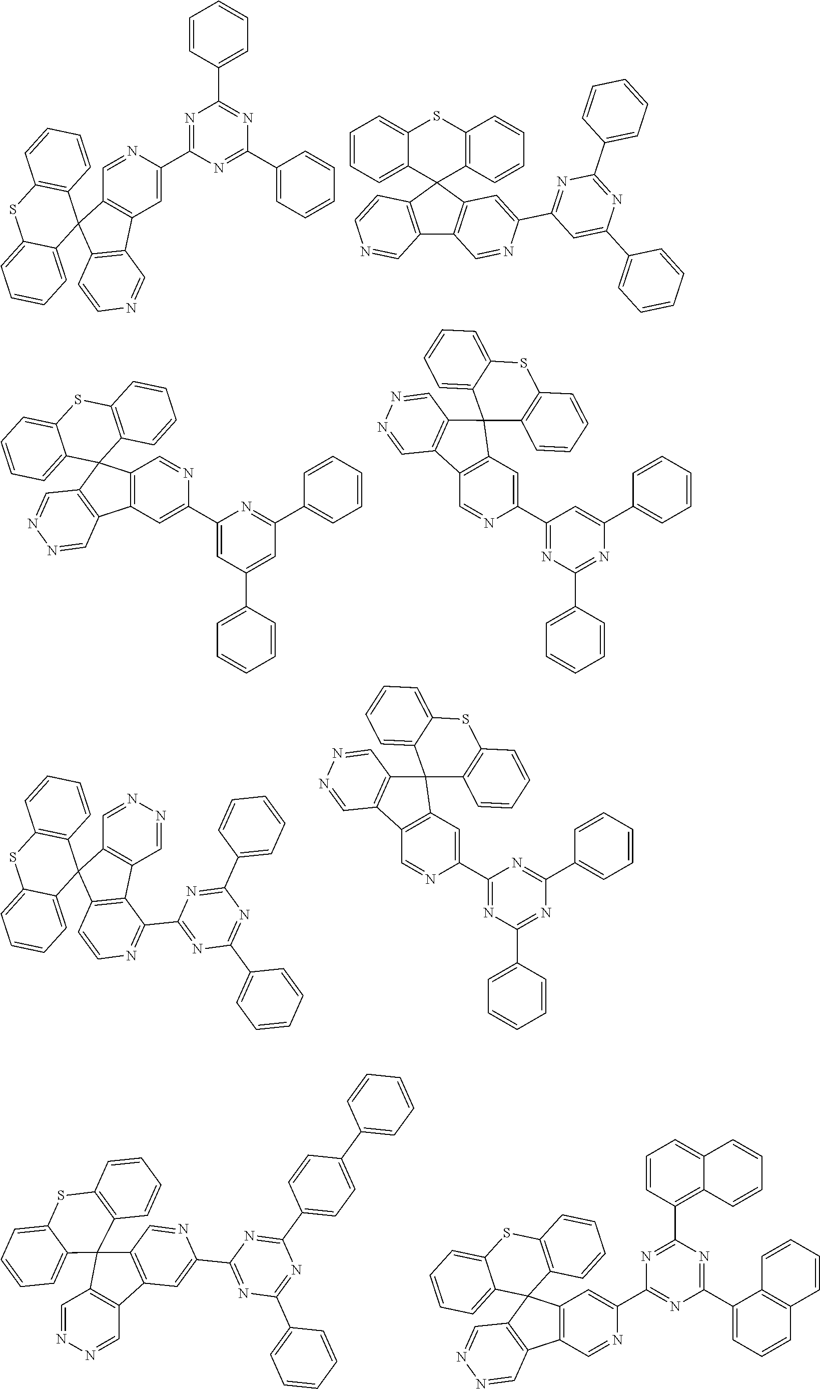

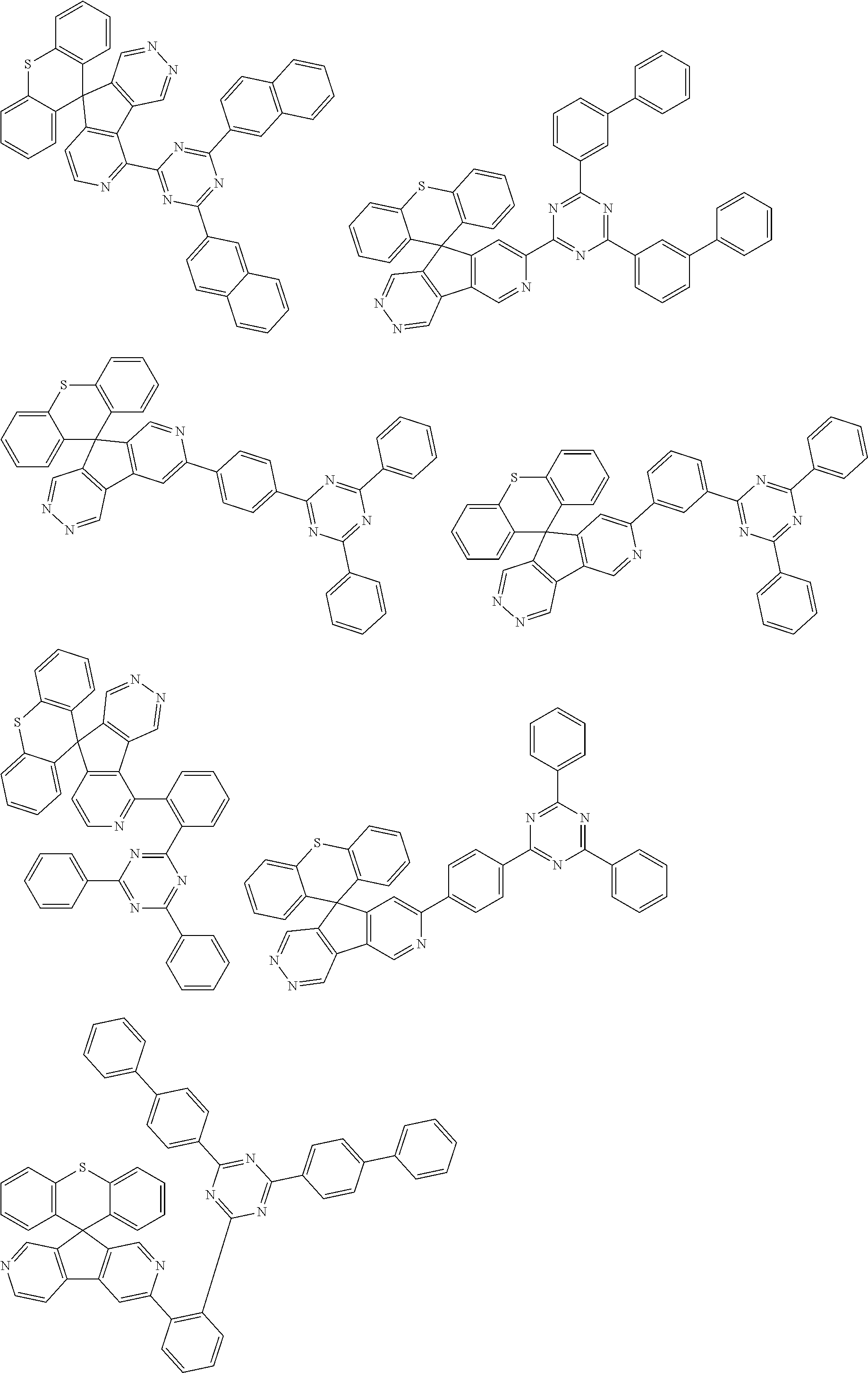

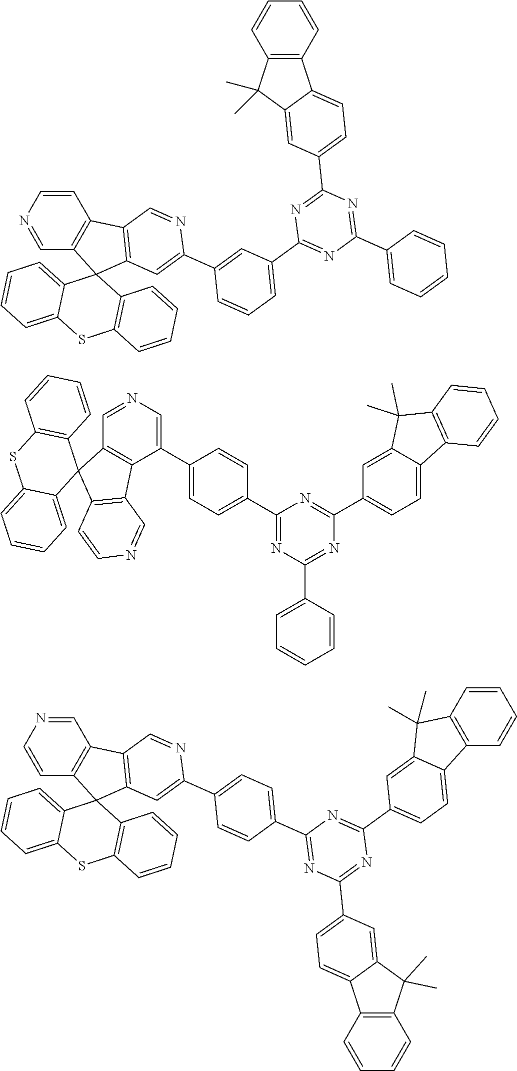

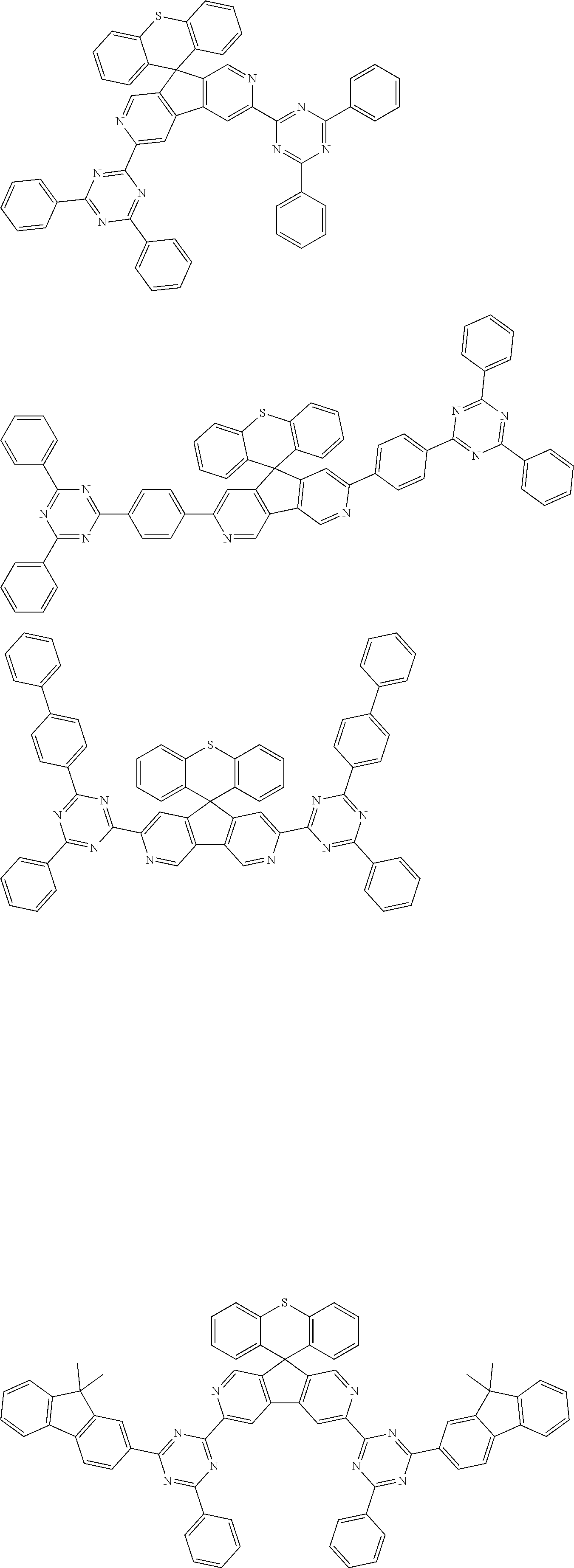

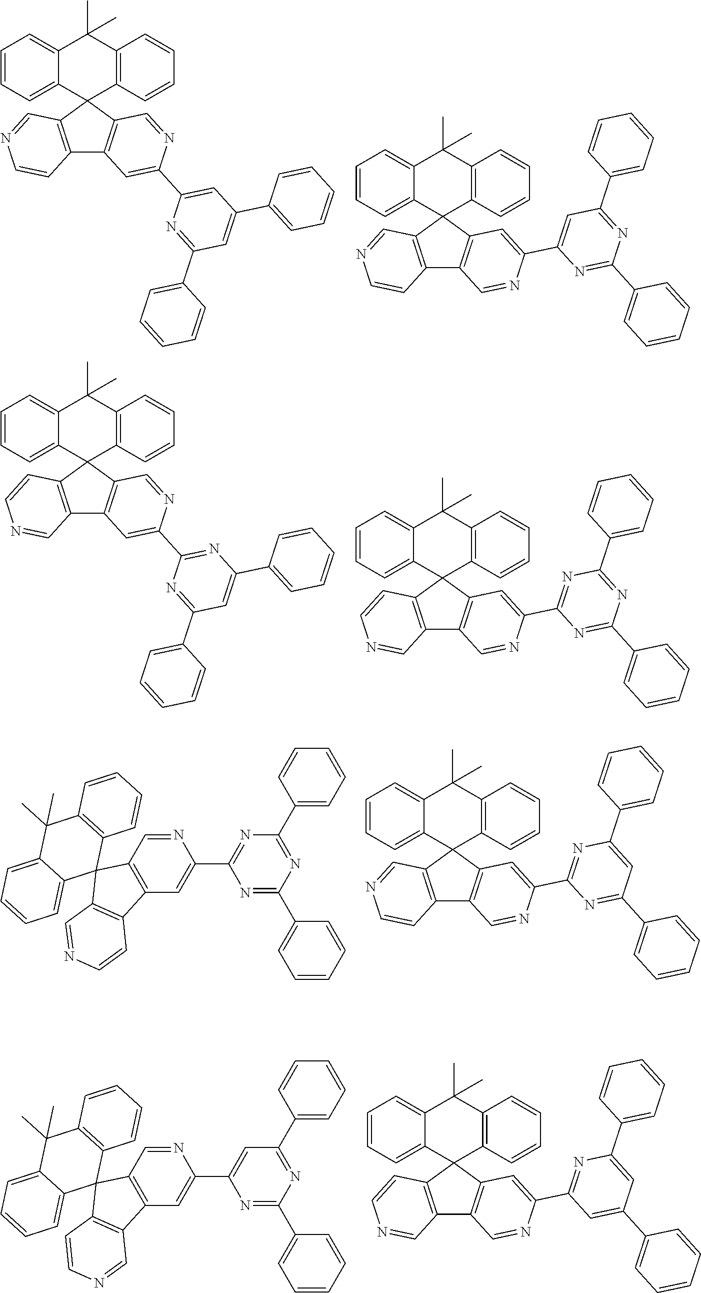

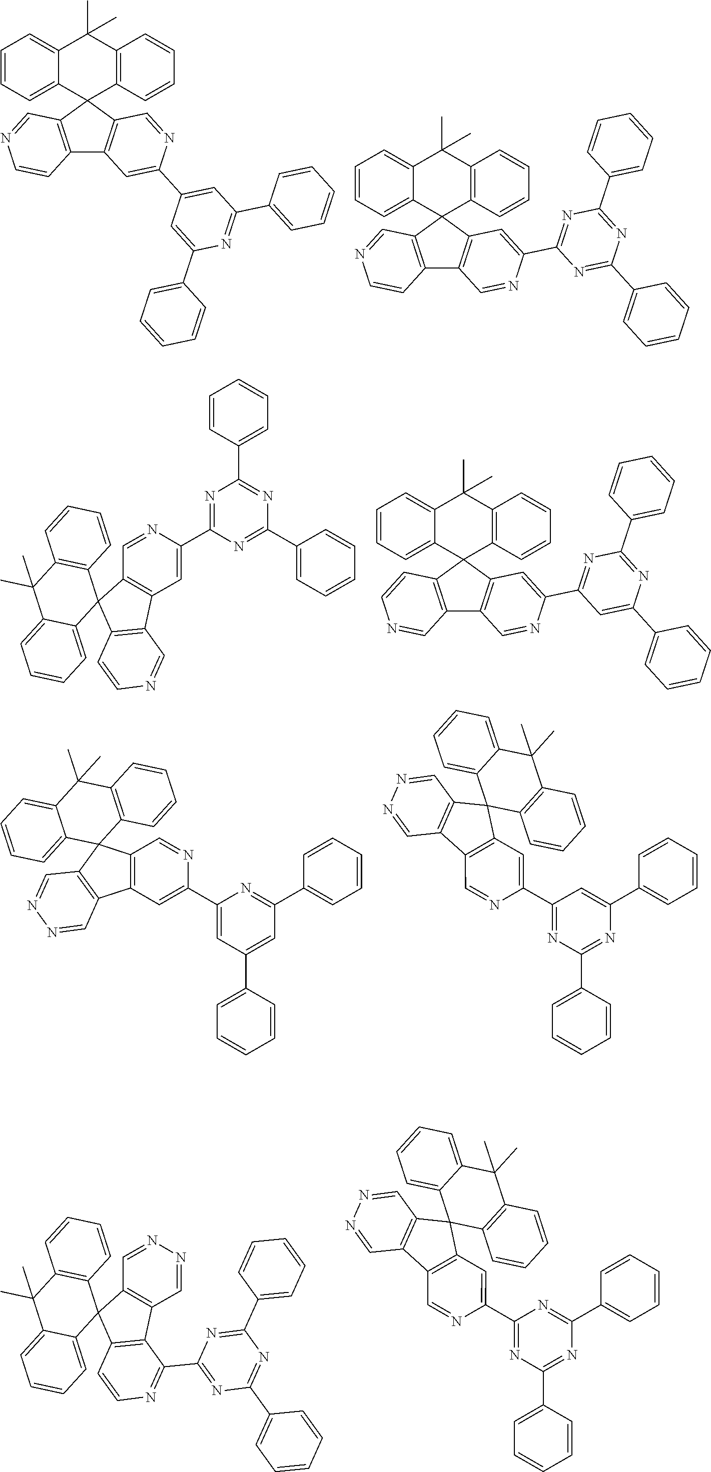

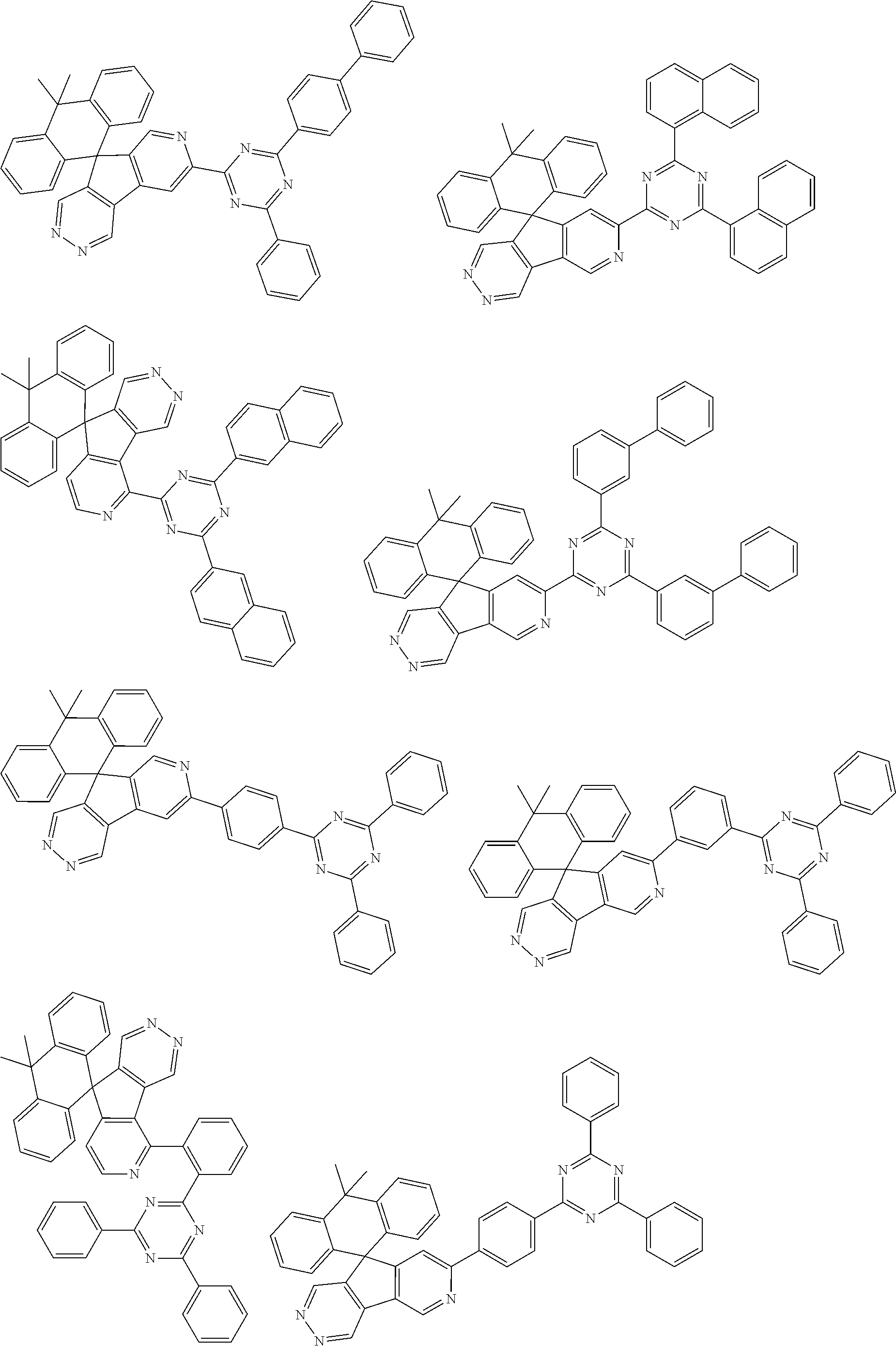

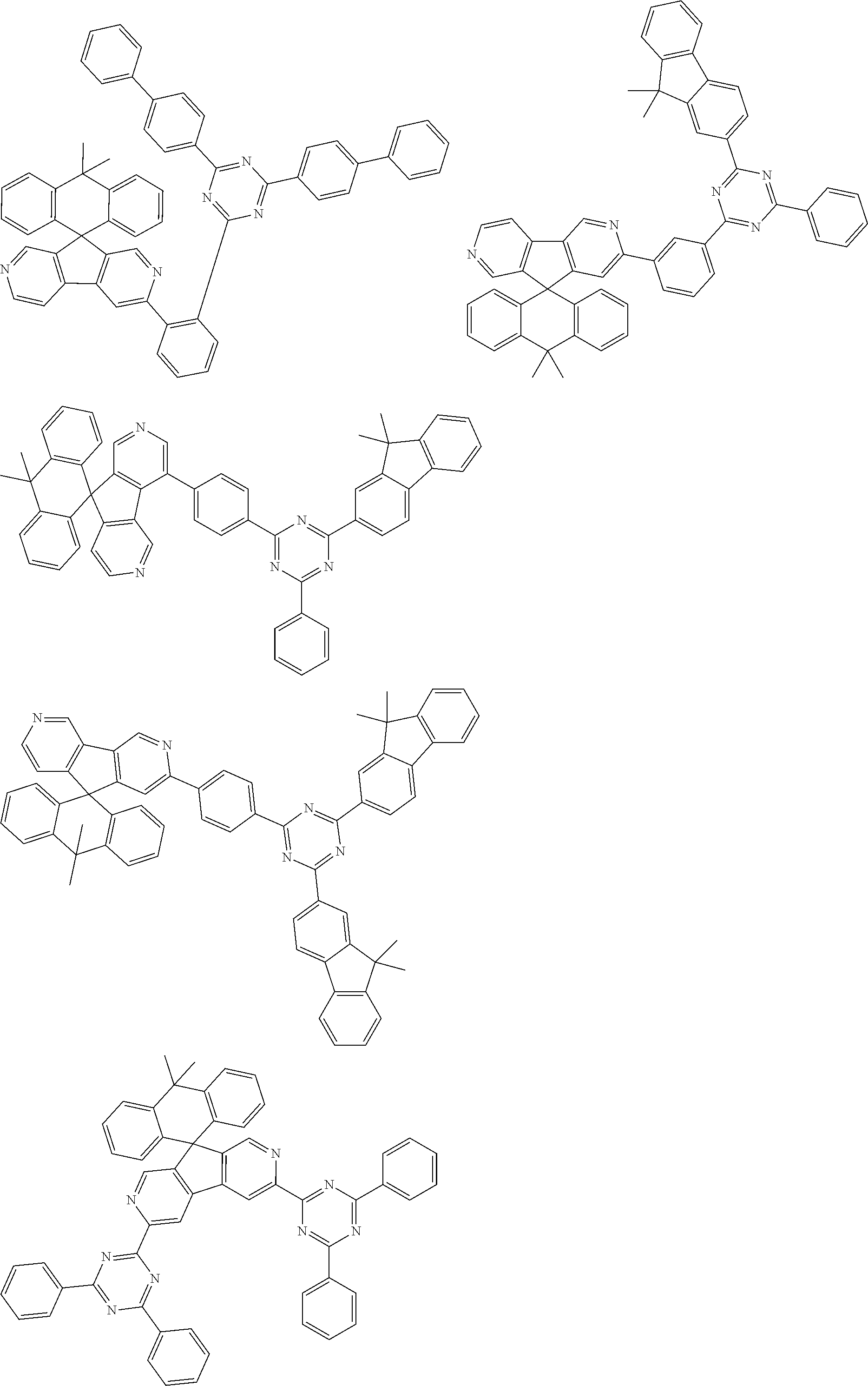

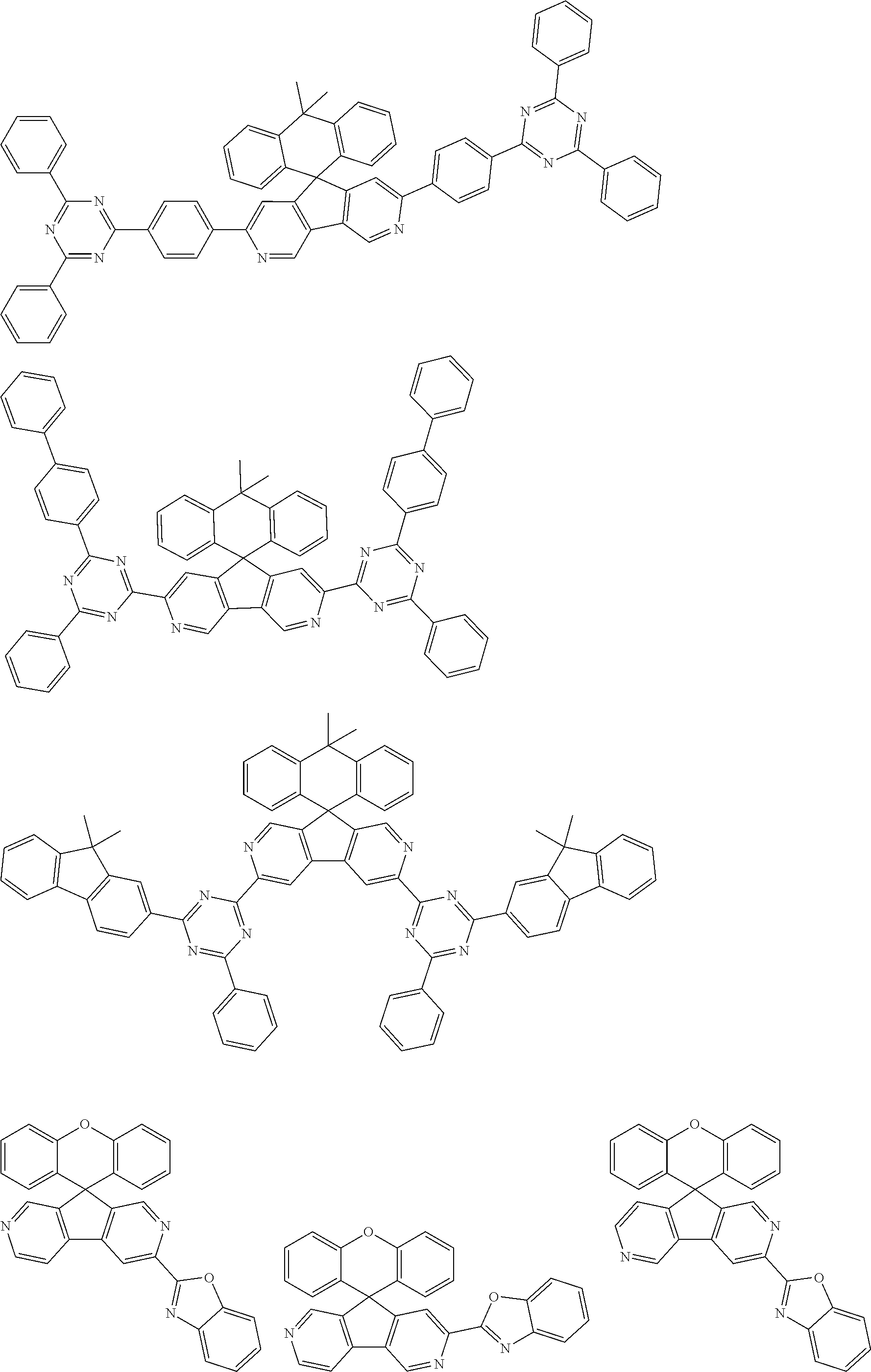

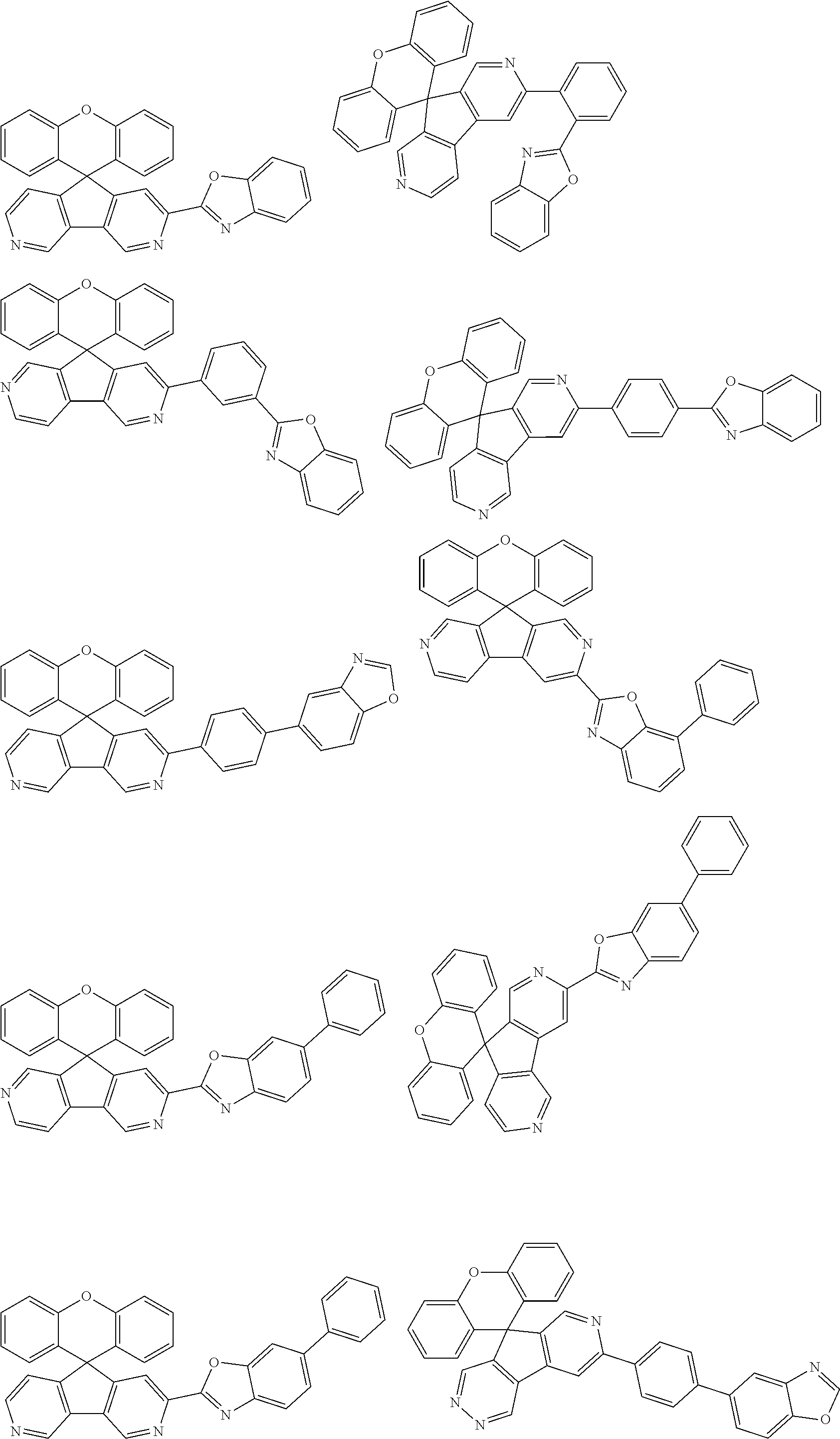

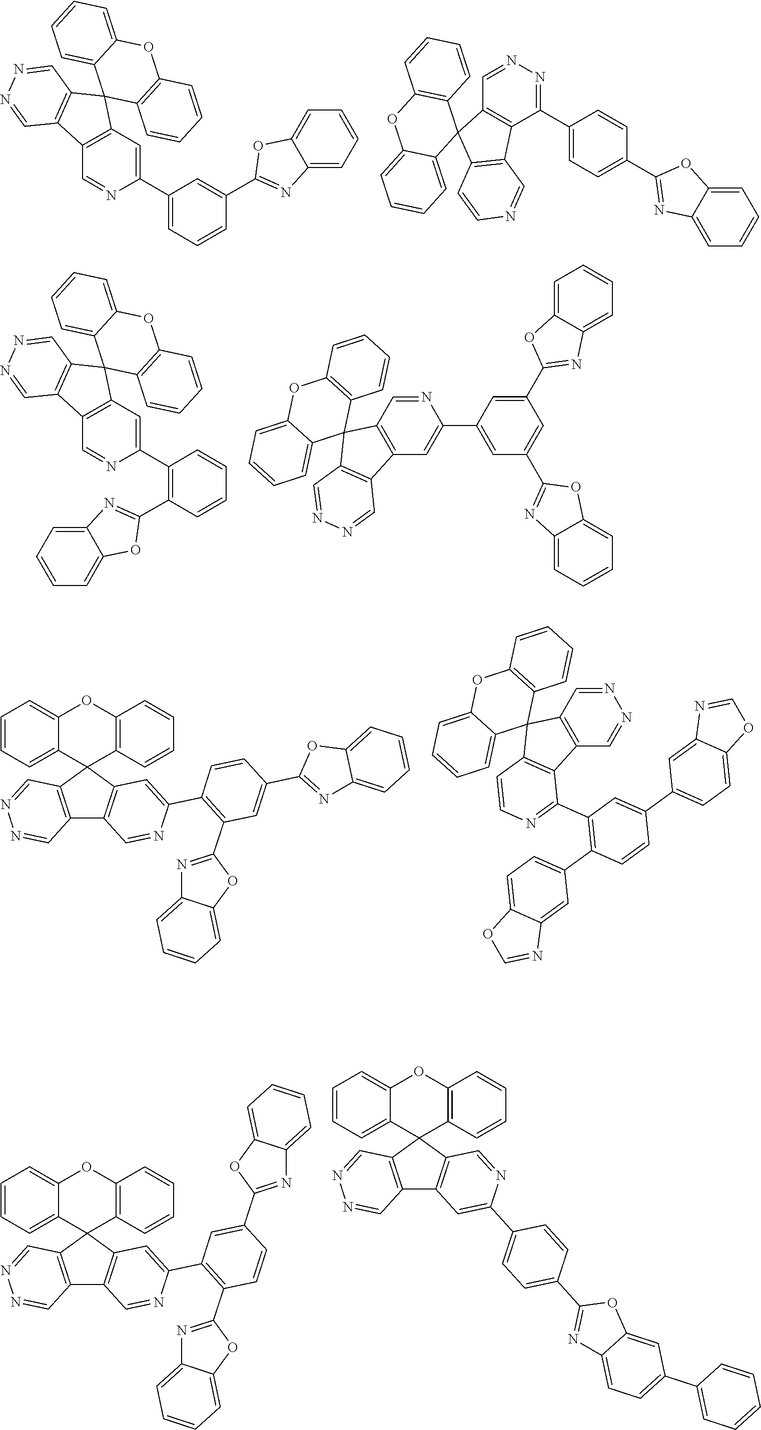

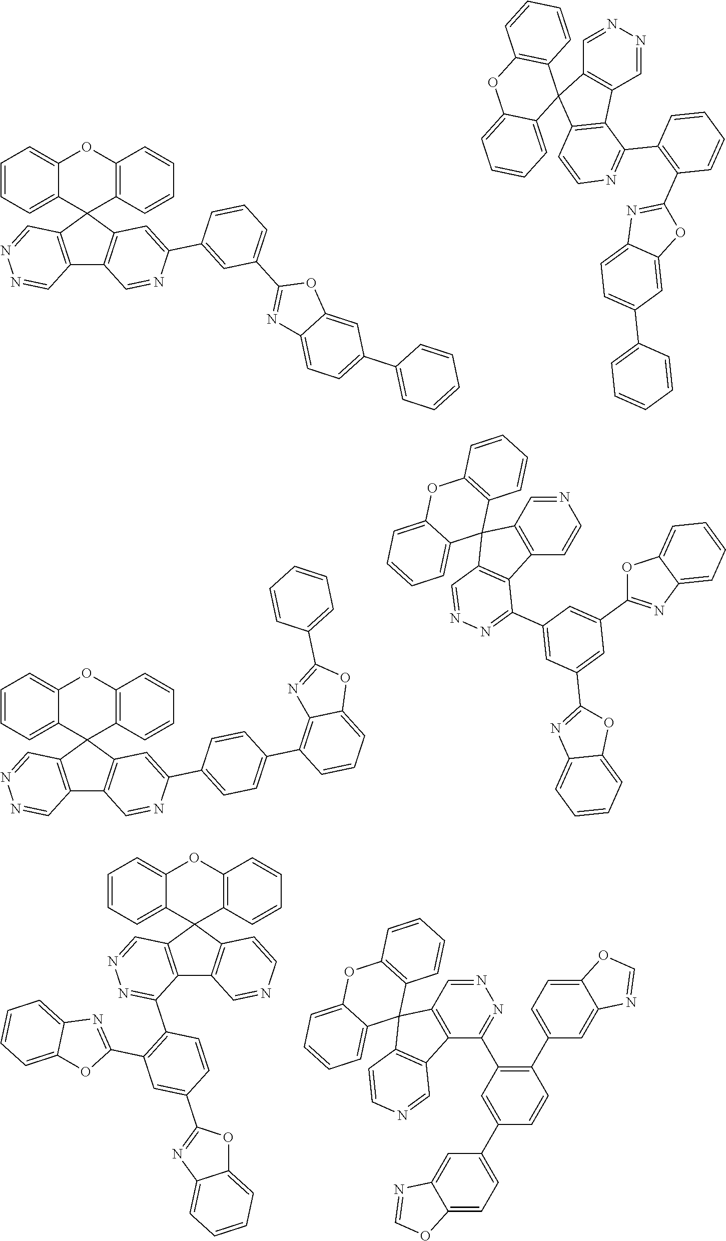

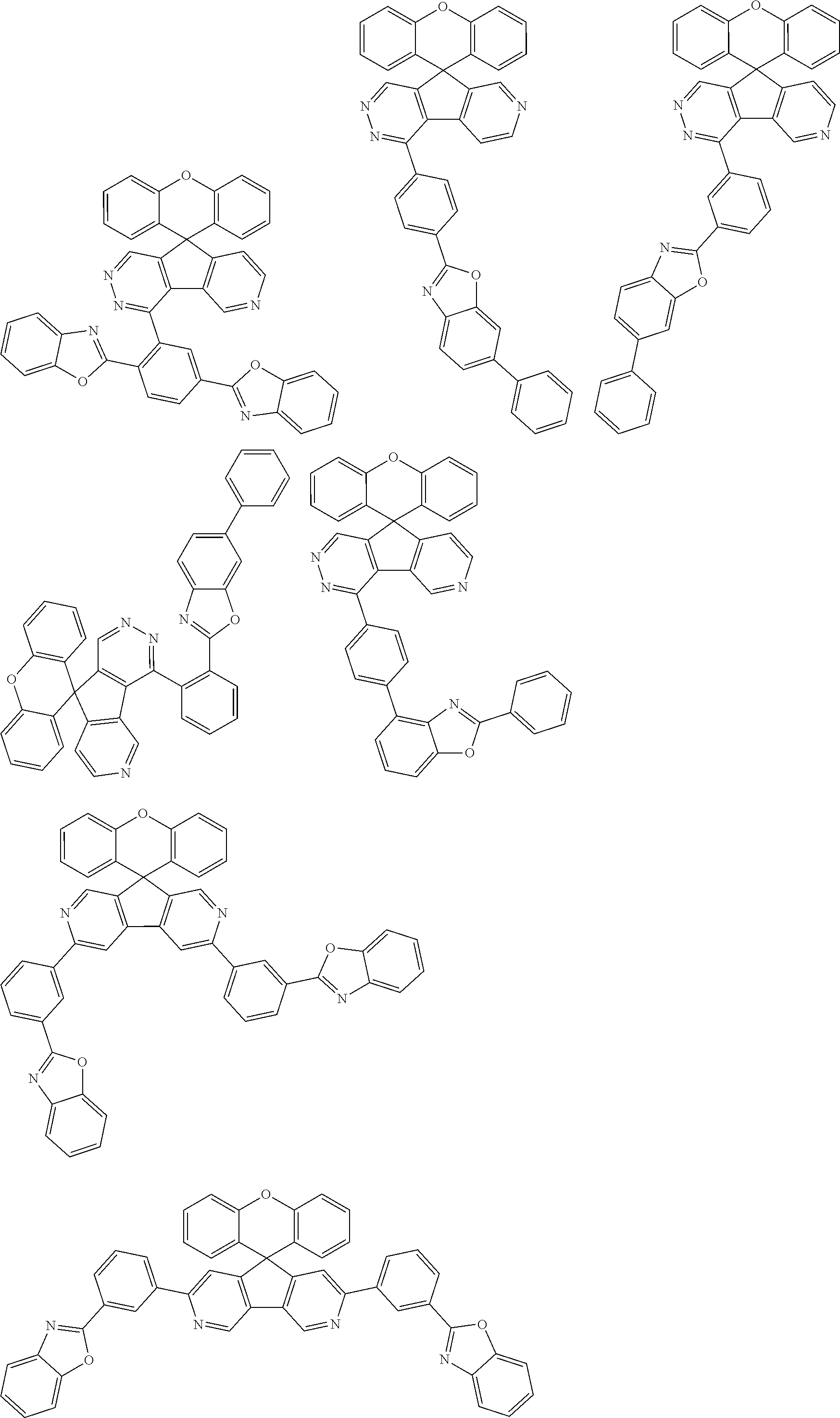

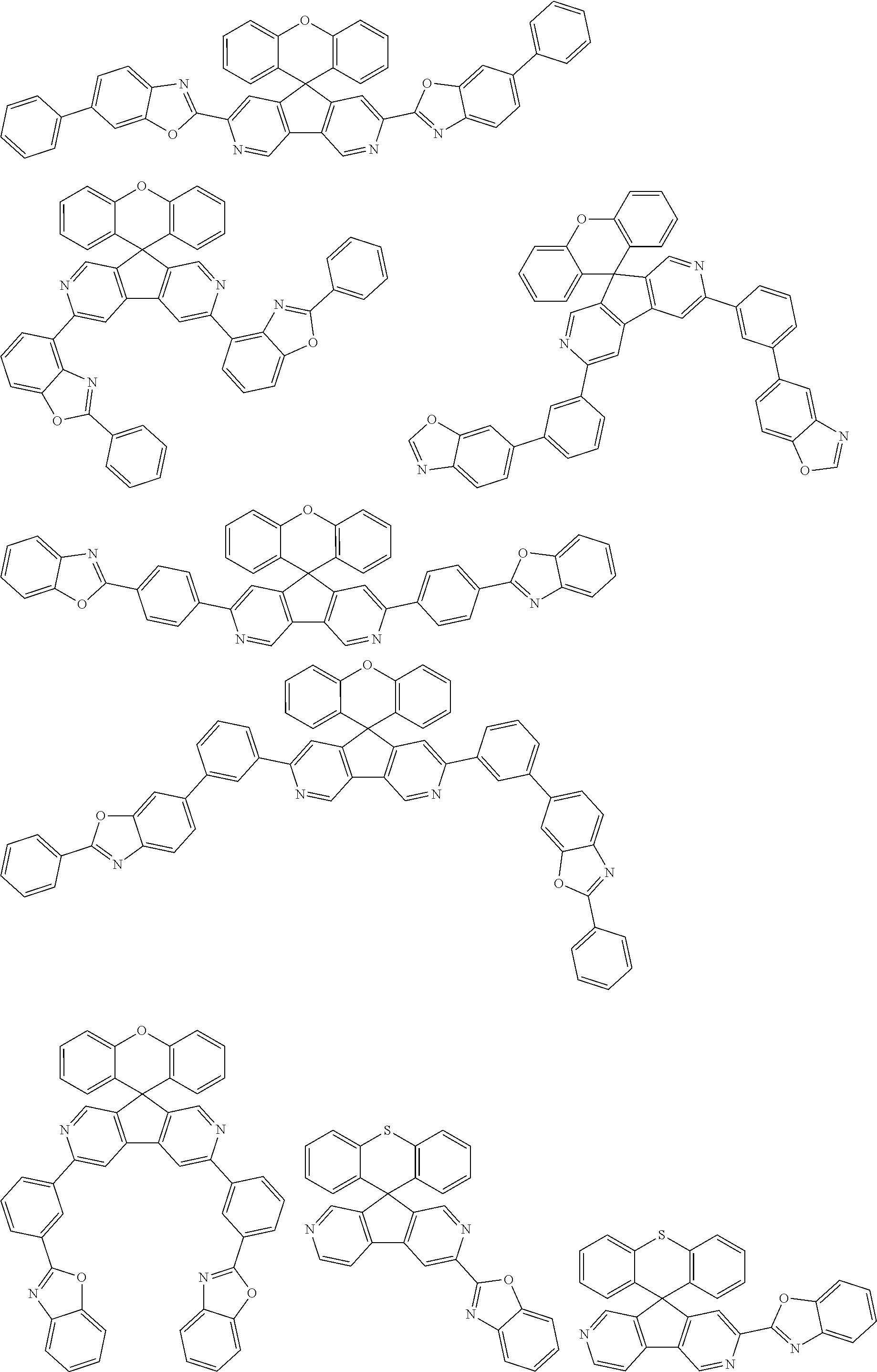

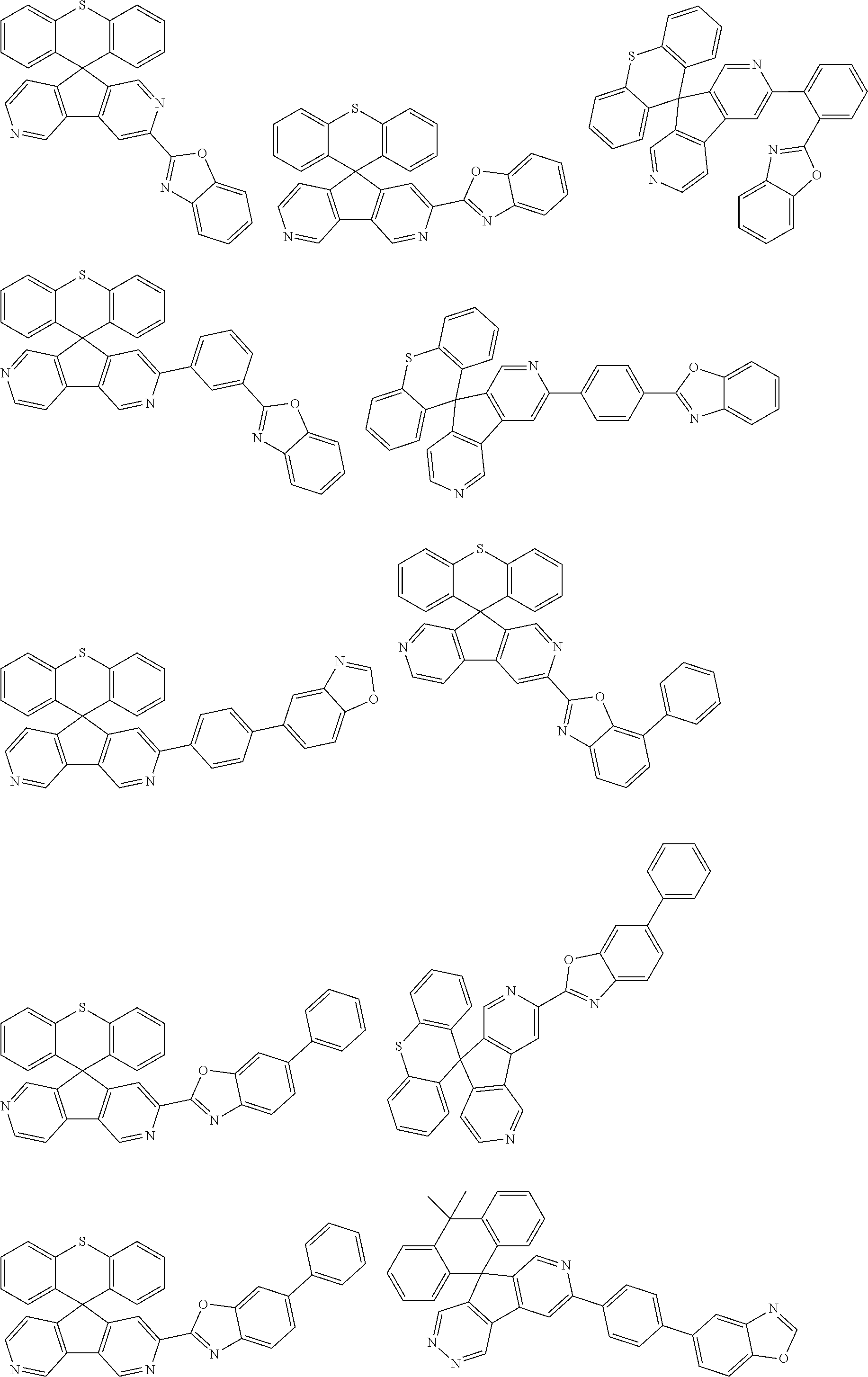

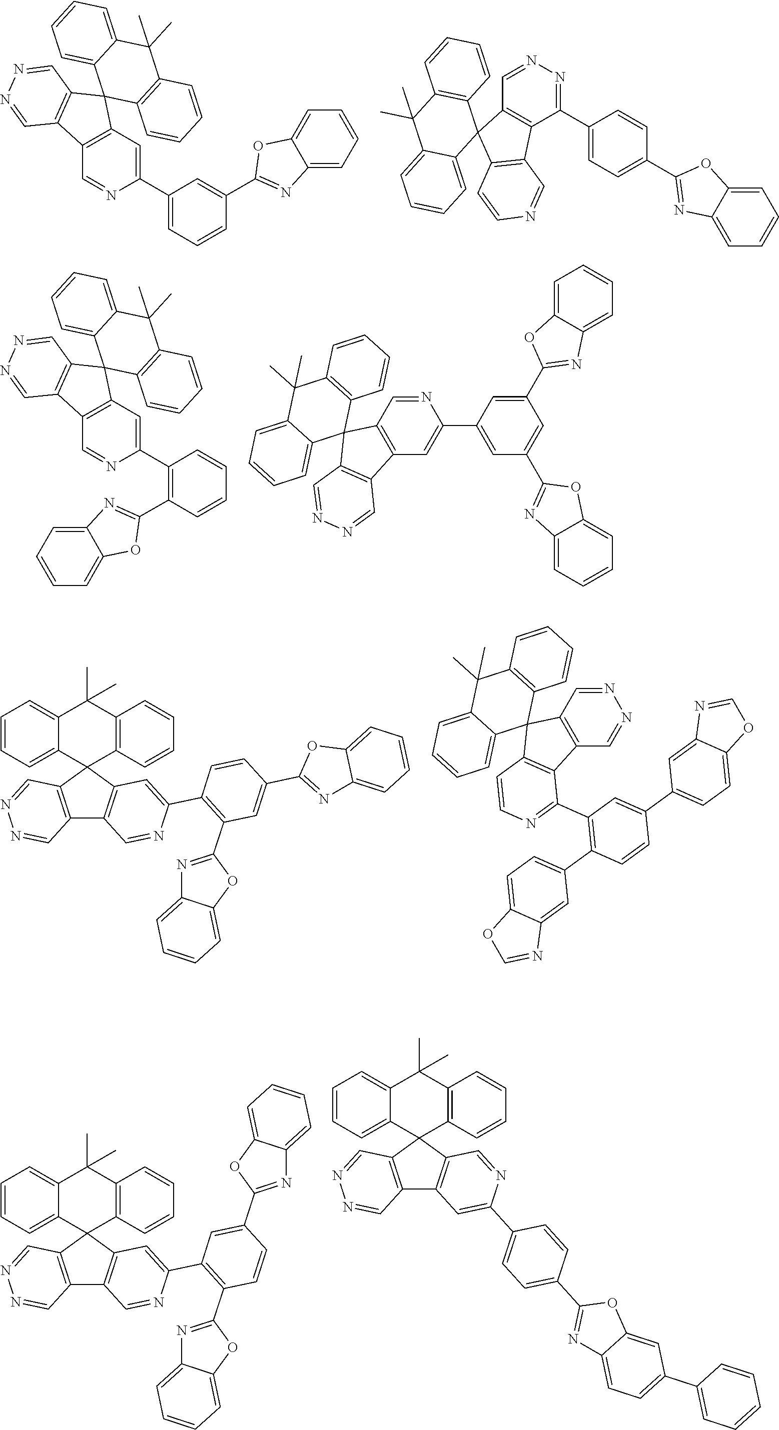

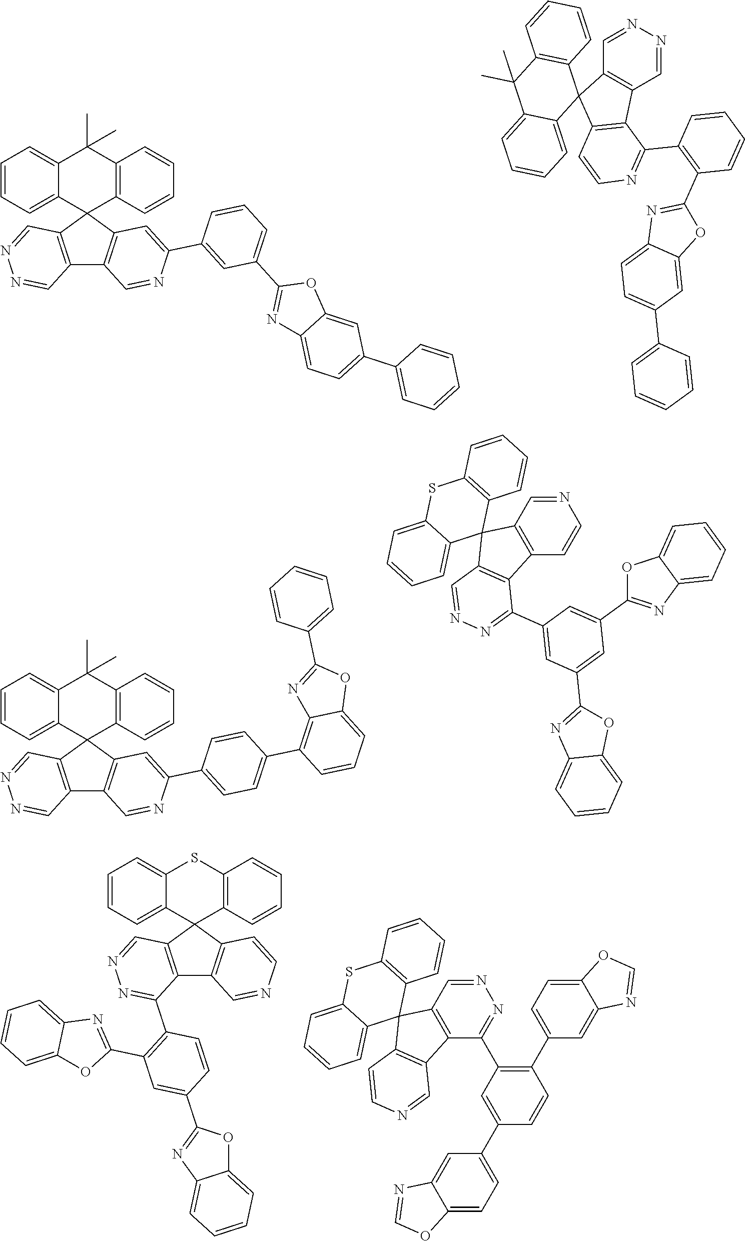

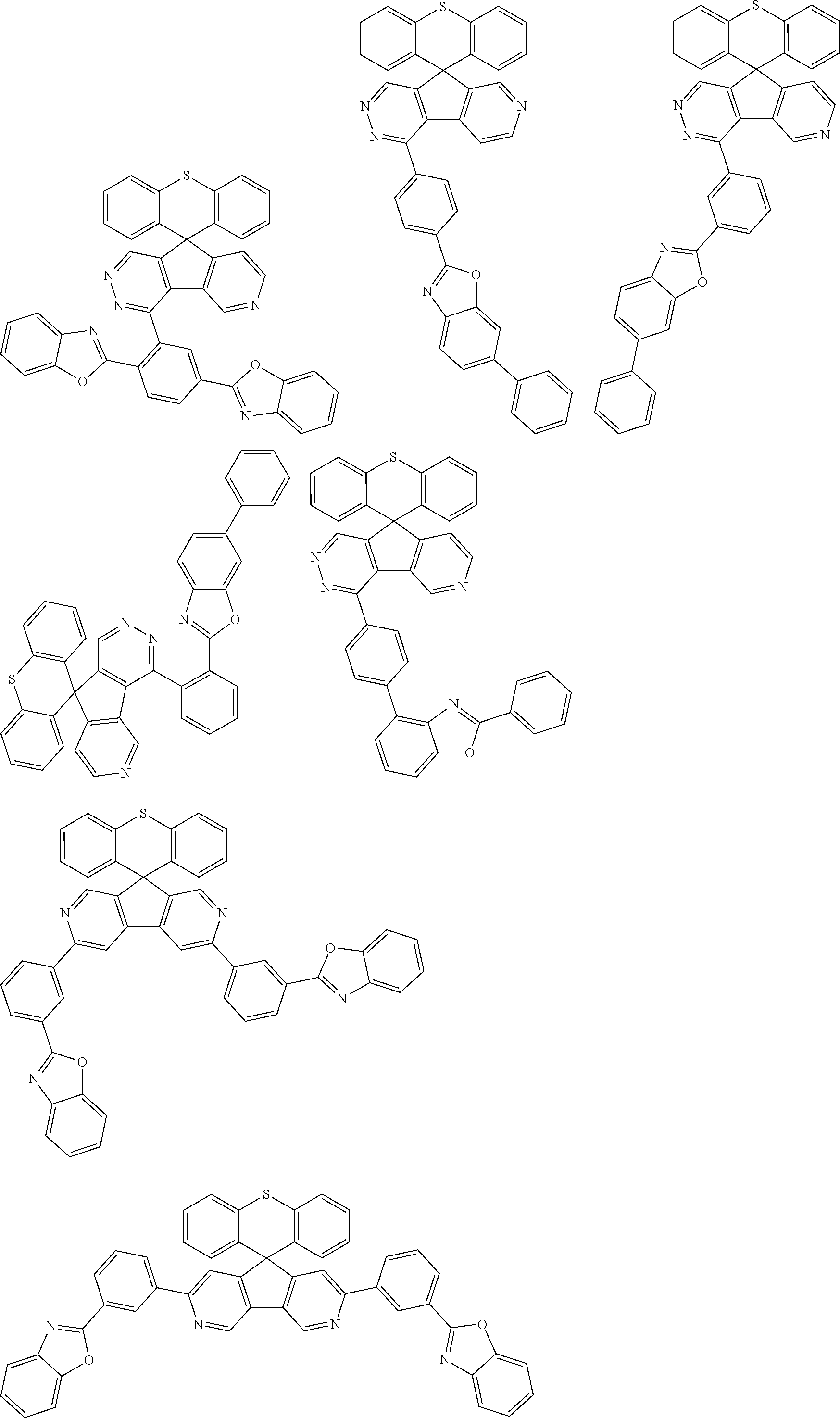

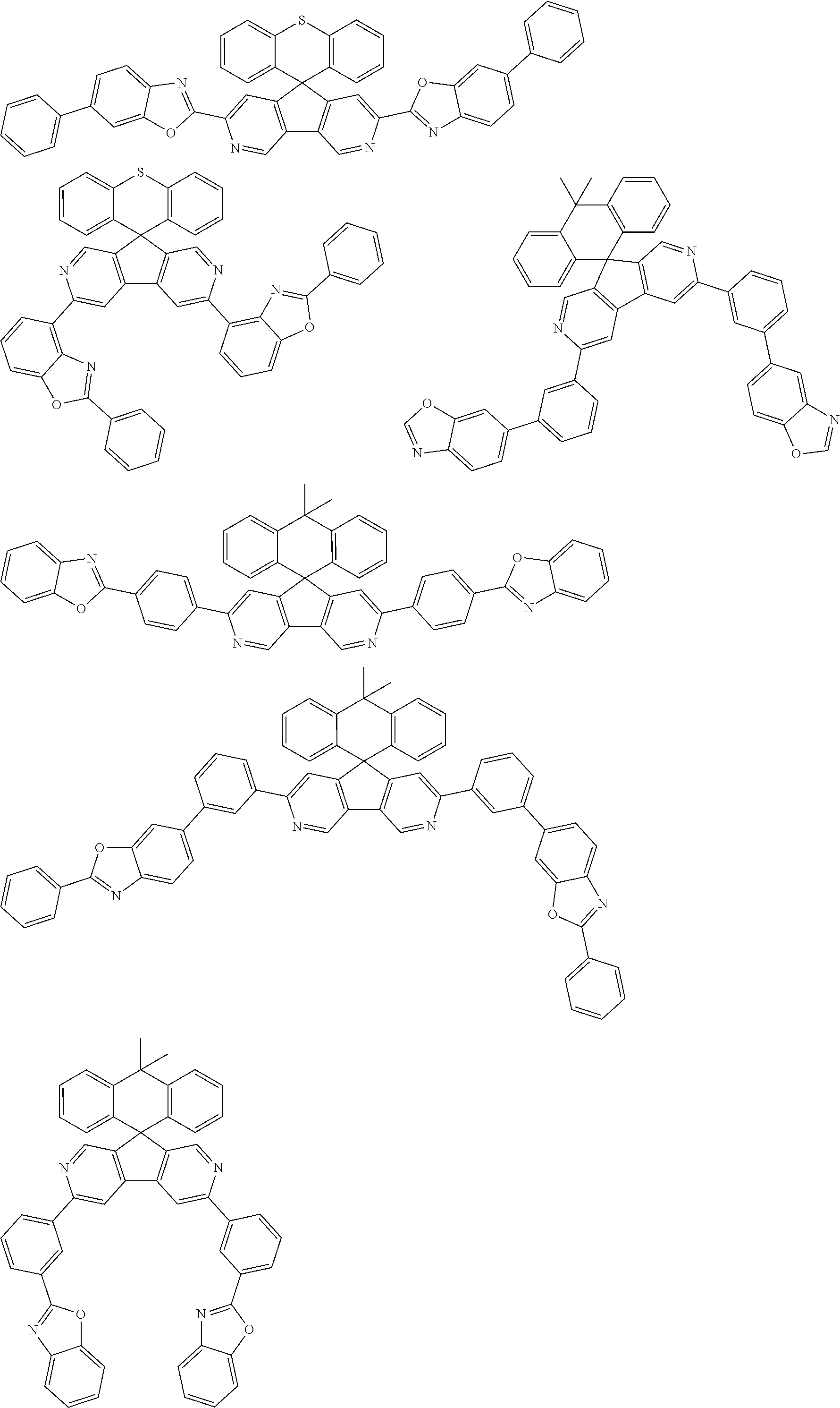

Illustratively, the following contents of the present disclosure provide methods for the synthesis of some compounds. Those skilled in the art can refer to the following methods to prepare other compounds, wherein, the raw materials not specified were all commercially available.

### Synthesis of Intermediates:

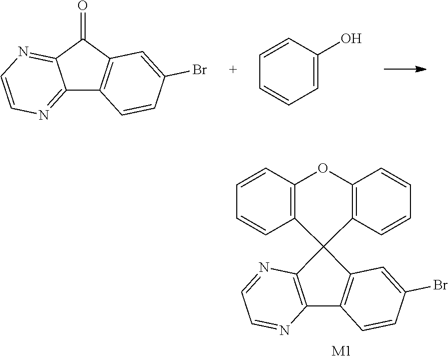

Synthesis of Intermediate M1: In a round-bottomed flask, 7-bromo-9H-indolo[1,2-b]pyrazin-9-one (5 g, 19.3 mmol) and phenol (2.18 g, 23.2 mmol) were added into a mixed solution of trifluoromethanesulfonic acid (6 g) and toluene (60 ml), heated to reflux and stirred for 12 h. The reaction was confirmed by thin layer chromatography (TLC) and terminated after adding water. The organic layer was extracted with dichloromethane and filtered under reduced pressure. The mixture was subjected to column purification and recrystallization to obtain intermediate compound M1 (5.20 g, yield: 65%). ¹H NMR (500 MHz, Chloroform) δ 8.58 (s, 1H), 8.54 (s, 1H), 8.21 (s, 1H), 7.57 (d, J=5.0 Hz, 2H), 7.31 (s, 2H), 7.18 (m, 4H), 7.00 (s, 2H).

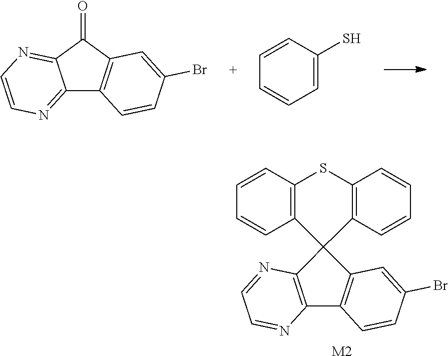

Synthesis of Intermediate M2: The synthesis of Intermediate M2 was similar to the synthesis of Intermediate M1, except that the benzene ring was replaced by thiophenol to obtain Intermediate M2. ¹H NMR (500 MHz, Chloroform) δ 8.58 (s, 1H), 8.54 (s, 1H), 8.21 (s, 1H), 7.69 (s, 2H), 7.57 (d, J=5.0 Hz, 2H), 7.33 (s, 1H), 7.03 (d, J=10.0 Hz, 4H).

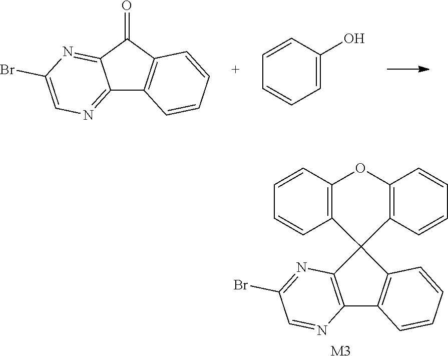

Synthesis of Intermediate M3: The synthesis of Intermediate M3 was similar to the synthesis of Intermediate M1, except that 7-bromo-9H-indolo[1,2-b]pyrazin-9-one was replaced by 2-bromo-9H-indolo[1,2-b]pyrazin-9-one to obtain Intermediate M3. ¹H NMR (500 MHz, Chloroform) δ 8.63 (s, 1H), 8.18 (s, 1H), 7.47 (d, J=5.0 Hz, 2H), 7.31 (s, 3H), 7.18 (m, 4H), 7.00 (s, 2H).

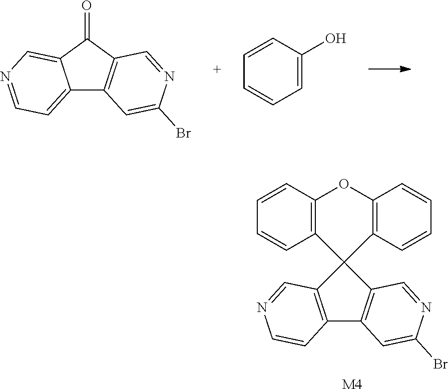 [SMILES: O=C1c2cnccc2-c2cc(Br)ncc21 ; Oc1ccccc1 ; Brc1cc2c(cn1)C1(c3ccccc3Oc3ccccc31)c1cnccc1-2 ; "M4"]

Synthesis of Intermediate M4: The synthesis of Intermediate M4 is similar to the synthesis of Intermediate M1, except that 7-bromo-9H-indolo[1,2-b]pyrazin-9-one was replaced by 3-bromo-9H-cyclopenta[1,2-c:4,3-c′]dipyridin-9-one to obtain Intermediate M4. ¹H NMR (500 MHz, Chloroform) δ 8.95 (s, 1H), 8.63 (s, 1H), 8.54 (s, 1H), 8.15 (s, 1H), 7.56 (s, 1H), 7.31 (s, 2H), 7.18 (m, 4H), 7.00 (s, 2H).

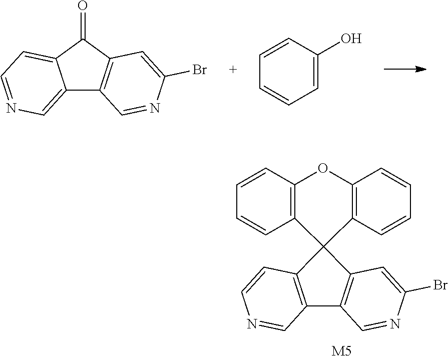

Synthesis of Intermediate M5: The synthesis of Intermediate M5 was similar to the synthesis of Intermediate M1, except that 7-bromo-9H-indolo[1,2-b]pyrazin-9-one was replaced by 3-bromo-5H-cyclopenta[2,1-c:3,4-c′]dipyridin-5-one to obtain Intermediate M5^{. 1}H NMR (500 MHz, Chloroform) δ 9.24 (s, 1H), 8.68 (s, 1H), 8.20 (s, 1H), 7.94 (s, 1H), 7.31 (s, 2H), 7.18 (m, 4H), 7.06 (s, 1H), 7.00 (s, 2H).

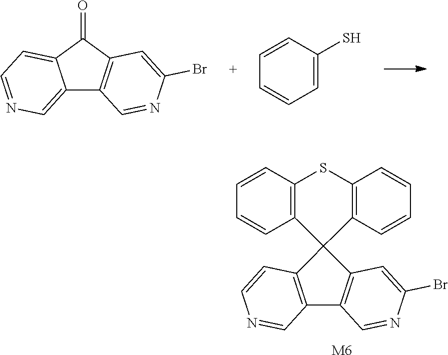

Synthesis of Intermediate M6: The synthesis of Intermediate M6 was similar to the synthesis of Intermediate M1, except that 7-bromo-9H-indolo[1,2-b]pyrazin-9-one was replaced by 3-bromo-5H-cyclopenta[2,1-c:3,4-c′]dipyridin-5-one, and benzene ring was replaced by thiophenol, to obtain Intermediate M6. ¹H NMR (500 MHz, Chloroform) δ 9.24 (s, 1H), 8.67 (s, 1H), 8.20 (s, 1H), 8.02 (s, 1H), 7.69 (s, 2H), 7.33 (s, 1H), 7.08-6.96 (m, 5H).

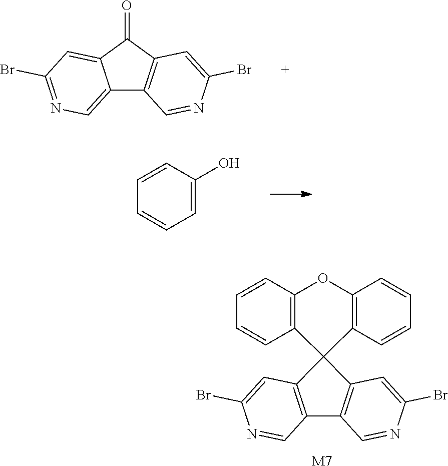

Synthesis of Intermediate M7: The synthesis of Intermediate M7 was similar to the synthesis of Intermediate M1, except that 7-bromo-9H-indolo[1,2-b]pyrazin-9-one was replaced by 3,7-dibromo-5H-cyclopenta[2,1-c:3,4-c′]dipyridin-5-one to obtain Intermediate M7. ¹H NMR (500 MHz, Chloroform) δ 8.67 (s, 1H), 7.94 (s, 1H), 7.31 (s, 1H), 7.18 (d, J=15.0 Hz, 2H), 7.00 (s, 1H)

### Synthesis of Compounds:

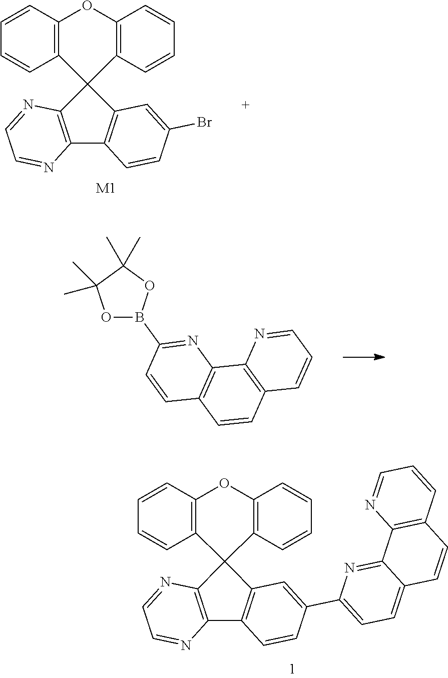

Synthesis of Compound 1: M1 (5 g, 12.14 mmol), 2-(4,4,5,5-tetramethyl-1,3,2-dioxaborolan-2-yl)-1,10-phenanthroline (3.80 g, 12.5 mmol), Pd(PPh₃)₄ (0.7 g, 0.6 mmol), potassium carbonate (2.76 g, 20 mmol) and THF/H₂O (60 mL/12 mL) were added to a 200 mL pressure bottle, heated under argon protection to 100° C. to perform pressurized reaction for 12 hours. After the reaction was completed, the reaction mixture was cooled to room temperature, and a large amount of solid precipitated. The reaction solution was filtered, and the filtered solid was washed with ethanol (100 mL) and dried to obtain a white powdery solid (4.66 g, 9.10 mmol) with a yield of 75%, ¹H NMR (500 MHz, Chloroform) δ 8.80 (s, 1H), 8.63 (s, 1H), 8.58 (s, 1H), 8.53 (m, 2H), 8.49-8.36 (m, 3H), 7.88 (s, 1H), 7.57 (d, J=10.0 Hz, 2H), 7.33 (m, 3H), 7.18 (m, 4H), 7.00 (s, 2H).

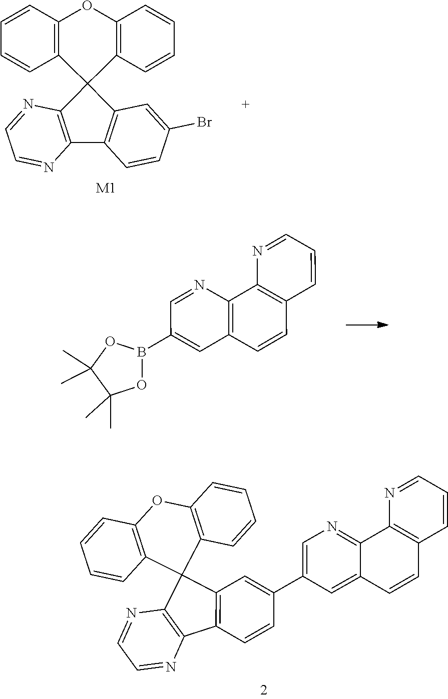

Synthesis of Compound 2: The synthesis of Compound 2 was similar to the synthesis of Compound 1, except that 2-(4,4,5,5-tetramethyl-1,3,2-dioxaborolan-2-yl)-1,10-phenanthroline was replaced by 3-(4,4,5,5-tetramethyl-1,3,2-dioxaborolan-2-yl)-1,10-phenanthroline to obtain Compound 2. ¹H NMR (500 MHz, Chloroform) δ 8.80 (s, 1H), 8.62-8.49 (m, 3H), 8.45 (d, J=3.2 Hz, 2H), 8.26 (s, 1H), 8.03 (s, 1H), 7.80 (s, 1H), 7.58 (m, 2H), 7.31 (s, 2H), 7.19 (t, J=7.5 Hz, 5H), 7.00 (s, 2H).

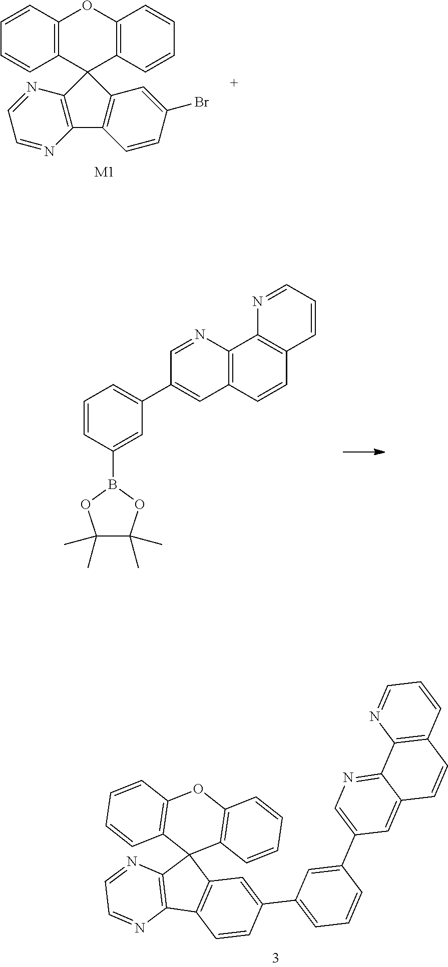

Synthesis of Compound 3: The synthesis of Compound 3 was similar to the synthesis of Compound 1, except that 2-(4,4,5,5-tetramethyl-1,3,2-dioxaborolan-2-yl)-1,10-phenanthroline was replaced by 3-(3-(4,4,5,5-tetramethyl-1,3,2-dioxaborolan-2-yl)phenyl)-1,10-phenanthroline to obtain Compound 3. ¹H NMR (500 MHz, Chloroform) δ 8.80 (s, 1H), 8.63-8.27 (m, 7H), 8.03 (s, 1H), 7.88 (s, 1H), 7.70 (s, 1H), 7.64-7.50 (m, 3H), 7.33 (m, 3H), 7.25-7.12 (m, 5H), 7.00 (s, 2H).

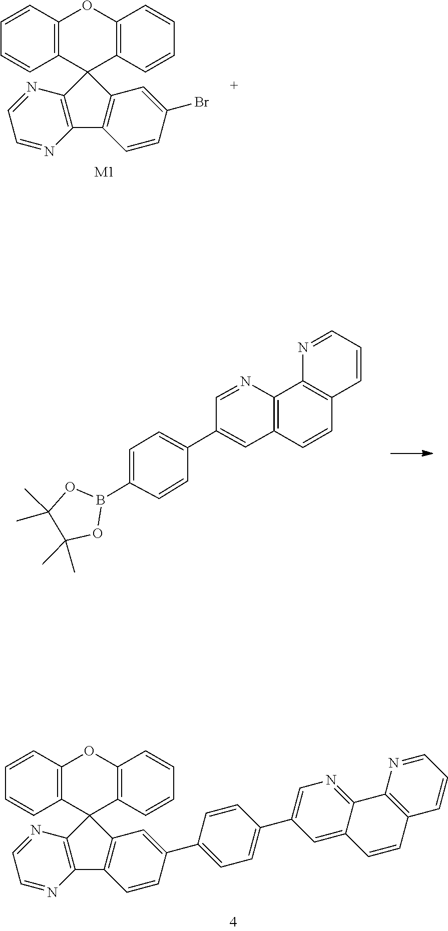

Synthesis of Compound 4: The synthesis of Compound 4 was similar to the synthesis of Compound 1, except that 2-(4,4,5,5-tetramethyl-1,3,2-dioxaborolan-2-yl)-1,10-phenanthroline was replaced by 3-(4-(4,4,5,5-tetramethyl-1,3,2-dioxaborolan-2-yl)phenyl)-1,10-phenanthroline to obtain Compound 4. ¹H NMR (500 MHz, Chloroform) δ 8.80 (s, 1H), 8.63-8.48 (m, 3H), 8.45 (d, J=1.0 Hz, 2H), 8.26 (s, 1H), 8.03 (s, 1H), 7.80 (s, 1H), 7.57 (m, 2H), 7.38-7.12 (m, 11H), 7.00 (s, 2H).

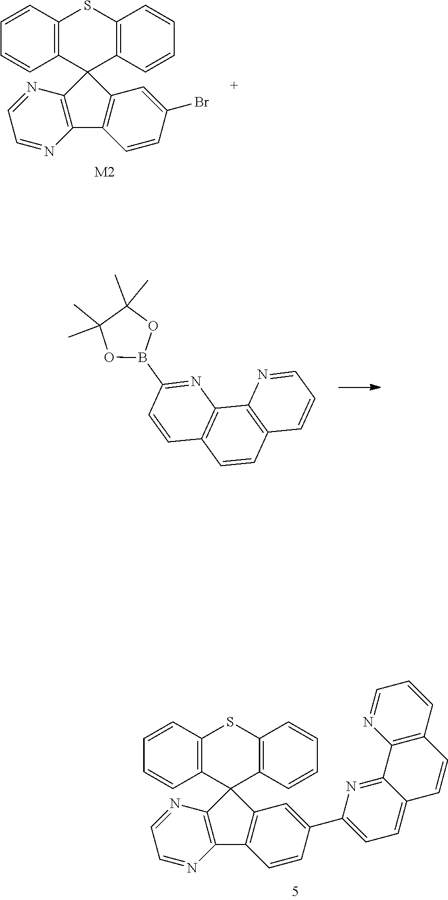

Synthesis of Compound 5: The synthesis of Compound 5 was similar to the synthesis of Compound 1, except that M1 was replaced by M2 to obtain Compound 5. ¹H NMR (500 MHz, Chloroform) δ 8.80 (s, 1H), 8.63 (s, 1H), 8.58 (s, 1H), 8.53 (d, J=15.0 Hz, 2H), 8.48-8.35 (m, 3H), 7.88 (s, 1H), 7.69 (s, 2H), 7.57 (d, J=10.0 Hz, 2H), 7.34 (d, J=5.0 Hz, 3H), 7.03 (d, J=10.0 Hz, 4H).

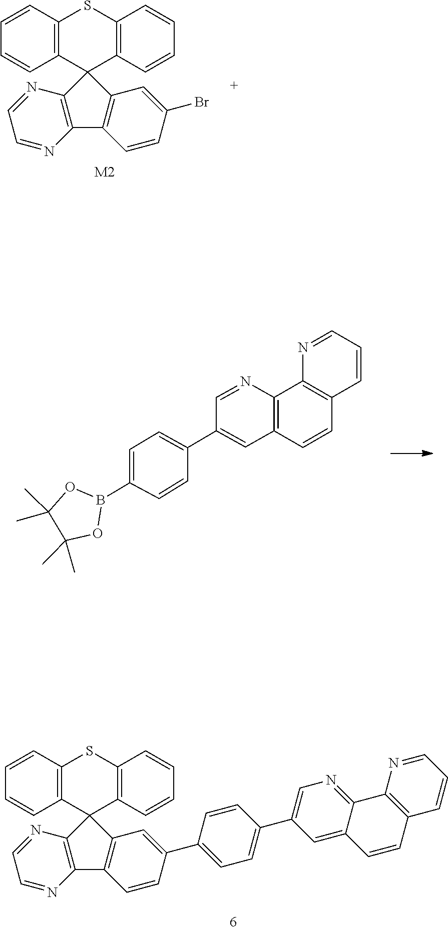

Synthesis of Compound 6: The synthesis of Compound 6 was similar to the synthesis of Compound 4, except that M1 was replaced by M2 to obtain Compound 6. ¹H NMR (500 MHz, Chloroform) δ 8.80 (s, 1H), 8.63-8.48 (m, 3H), 8.45 (d, J=2.5 Hz, 2H), 8.26 (s, 1H), 8.03 (s, 1H), 7.80 (s, 1H), 7.69 (s, 2H), 7.58 (d, J=15.0 Hz, 2H), 7.33 (s, 2H), 7.25 (s, 4H), 7.19 (s, 1H), 7.03 (d, J=10.0 Hz, 4H).

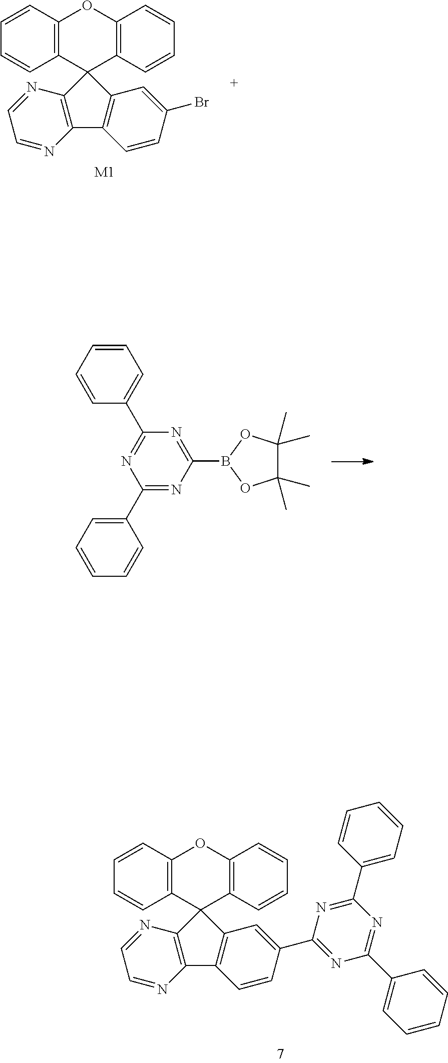

Synthesis of Compound 7: The synthesis of Compound 7 was similar to the synthesis of Compound 1, except that 2-(4,4,5,5-tetramethyl-1,3,2-dioxaborolan-2-yl)-1,10-phenanthroline was replaced by 2,4-diphenyl-6-(4,4,5,5-tetramethyl-1,3,2-dioxaborolan-2-yl)-1,3,5-triazine to obtain Compound 7. ¹H NMR (500 MHz, Chloroform) δ 8.58 (s, 1H), 8.53 (d, J=15.0 Hz, 2H), 8.36 (s, 4H), 8.03 (s, 1H), 7.90 (s, 1H), 7.50 (s, 6H), 7.31 (s, 2H), 7.18 (d. J=15.0 Hz, 4H), 7.00 (s, 2H).

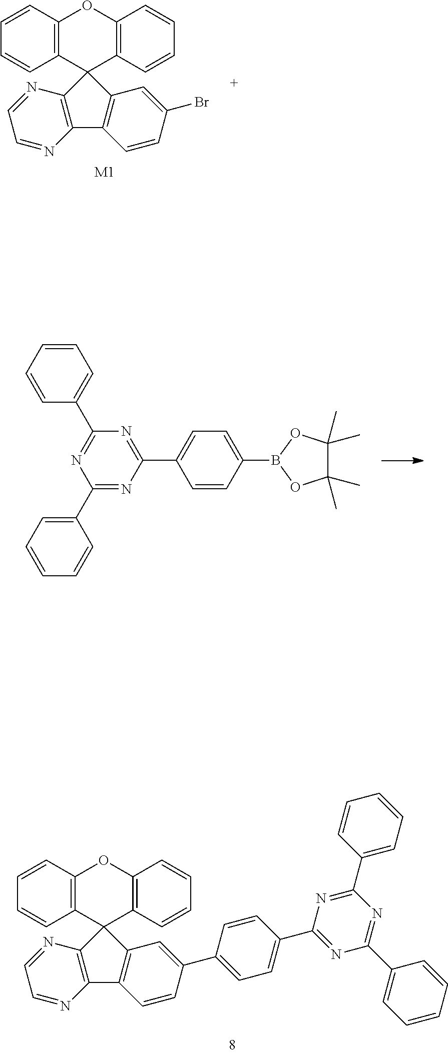

Synthesis of Compound 8: The synthesis of Compound 8 was similar to the synthesis of Compound 1, except that 2-(4,4,5,5-tetramethyl-1,3,2-dioxaborolan-2-yl)-1,10-phenanthroline was replaced by 2,4-diphenyl-6-(4-(4,4,5,5-tetramethyl-1,3,2-dioxaborolan-2-yl)phenyl)-1,3,5-triazine to obtain Compound 8. ¹H NMR (500 MHz, Chloroform) δ 8.65-8.47 (m, 3H), 8.36 (s, 4H), 8.03 (s, 1H), 7.96 (s, 2H), 7.50 (s, 6H), 7.38-7.11 (m, 9H), 7.00 (s, 2H).

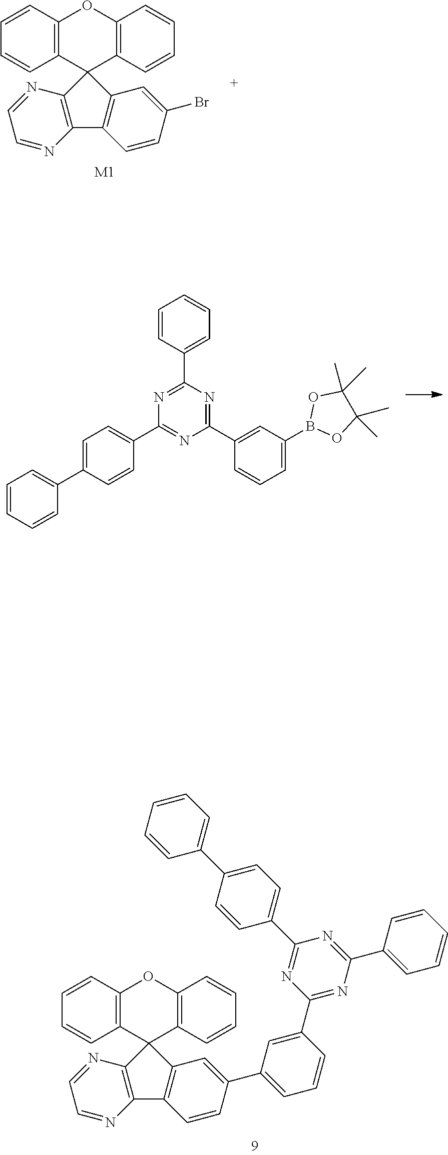

Synthesis of Compound 9: The synthesis of Compound 9 was similar to the synthesis of Compound 1, except that 2-(4,4,5,5-tetramethyl-1,3,2-dioxaborolan-2-yl)-1,10-phenanthroline was replaced by 2-([1,1′-diphenyl]-4-yl)-4-phenyl-6-(3-(4,4,5,5-tetramethyl-1,3,2-dioxaborolan-2-yl)phenyl)-1,3,5-triazine to obtain Compound 9. ¹H NMR (500 MHz, Chloroform) δ 8.62-8.47 (m. 3H), 8.37 (d, J=10.0 Hz, 3H), 8.29 (s, 1H), 8.03 (s, 1H), 7.96 (s, 2H), 7.72 (m, 3H), 7.61 (S, 1H), 7.55-7.36 (m, 6H), 7.35-7.12 (m, 9H), 7.00 (s, 2H).

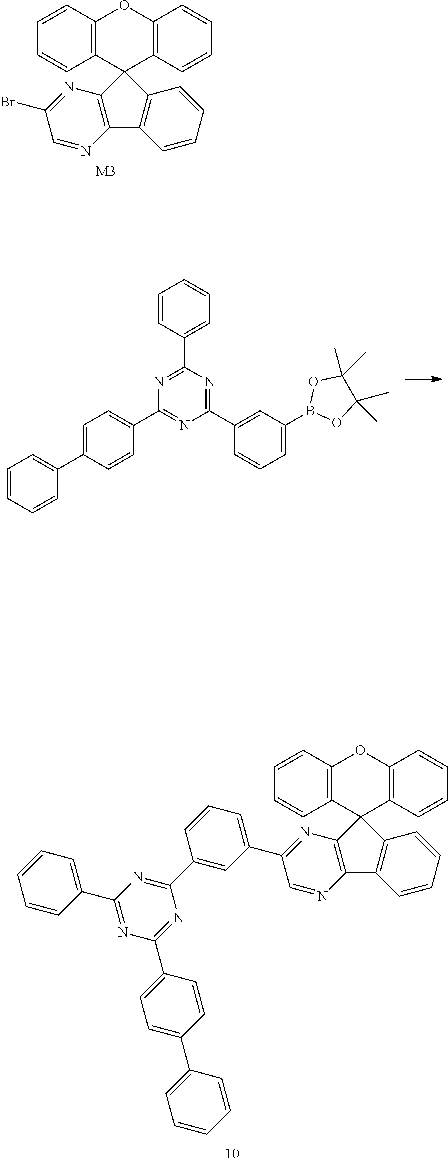

Synthesis of Compound 10: The synthesis of Compound 10 was similar to the synthesis of Compound 1, except that M1 was replaced by M3, and 2-(4,4,5,5-tetramethyl-1,3,2-dioxaborolan-2-yl)-1,10-phenanthroline was replaced by 2-([1,1′-diphenyl]-4-yl)-4-phenyl-6-(3-(4,4,5,5-tetramethyl-1,3,2-dioxaborolan-2-yl)phenyl)-1,3,5-triazine, to obtain Compound 10. ¹H NMR (500 MHz, Chloroform) δ 8.73 (s, 1H), 8.56-8.44 (m, 3H), 8.36 (s, 2H), 8.18 (s, 1H), 7.96 (s, 2H), 7.74 (d, J=5.0 Hz, 3H), 7.56-7.39 (m, 8H), 7.38-7.13 (m, 9H), 7.00 (s, 2H).

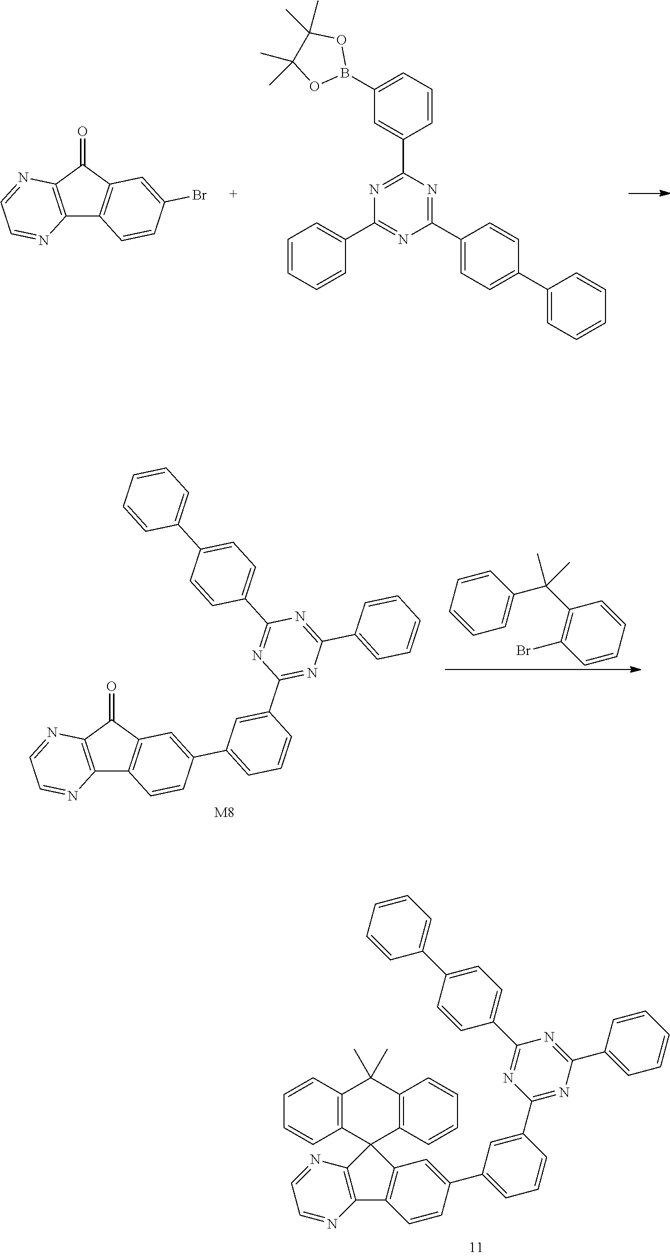

Synthesis of Intermediate M8: 7-Bromo-9H-indolo[1,2-b]pyrazol-9-one (2.08 g, 8 mmol), 2-([1,1′-diphenyl]-4-yl)-4-phenyl-6-(3-(4,4,5,5-tetramethyl-1,3,2-dioxaborolan-2-yl)phenyl)-1,3,5-triazine (4.6 g, 9 mmol), Pd(PPh₃)₄ (0.58 g, 0.5 mmol), potassium carbonate (2.76 g, 20 mmol) and THF/H₂O (60 mL/12 mL) were added to a 200 mL pressure bottle, heated under argon protection to 100° C. to perform pressurized reaction for 12 hours. After the reaction was completed, the reaction mixture was cooled to room temperature and a large amount of solid precipitated. The reaction solution was filtered, and the filtered solid was washed with ethanol (100 mL) and dried to obtain a yellow powdery solid (3.25 g, 5.76 mmol) with a yield of 72%. ¹H NMR (500 MHz, Chloroform) δ 8.83-8.66 (m, 3H), 8.43-8.30 (m, 4H), 8.02 (s, 1H), 7.96 (s, 2H), 7.72 (d, 3H), 7.59 (d, 2H), 7.54-7.36 (m, 6H), 7.25 (s, 1H), 7.25-7.21 (m, 1H).

Synthesis of Compound 11:1-Bromo-2-(2-isopropylbenzen-2-yl)benzene (1.65 g, 6 mmol) was dissolved in 30 mL of anhydrous tetrahydrofuran, added to a double-necked flask under argon atmosphere, cooled to −78° C. and stirred for 10 min, slowly added with n-butyllithium (1.6 mol/L, THF, 4.5 mL) dropwise by a syringe, stirred at −78° C. for 1 h, and then M8 (2.82 g, 5 mmol) was weighed and dissolved in anhydrous tetrahydrofuran and added to the flask, the mixture was heated to room temperature, and the reaction was continued under stirring for 24 hours under the protection of argon. A small amount of deionized water was added for quenching, and then the reaction was monitored with a TLC plate until the reaction was complete. The reaction liquid was rotary-evaporated to dryness, slurried and separated by a silica gel column chromatography to obtain a solid powder product (2.04 g, 2.75 mmol) with a yield of 55%, ¹H NMR (500 MHz, Chloroform) δ 8.64-8.47 (m, 3H), 8.37 (d, J=10.0 Hz, 3H), 8.29 (s, 1H), 8.03 (s, 1H), 7.96 (s, 2H), 7.72 (m, 3H), 7.61 (s, 1H), 7.57-7.35 (m, 10H), 7.31-7.10 (m, 7H), 1.69 (s, 6H).

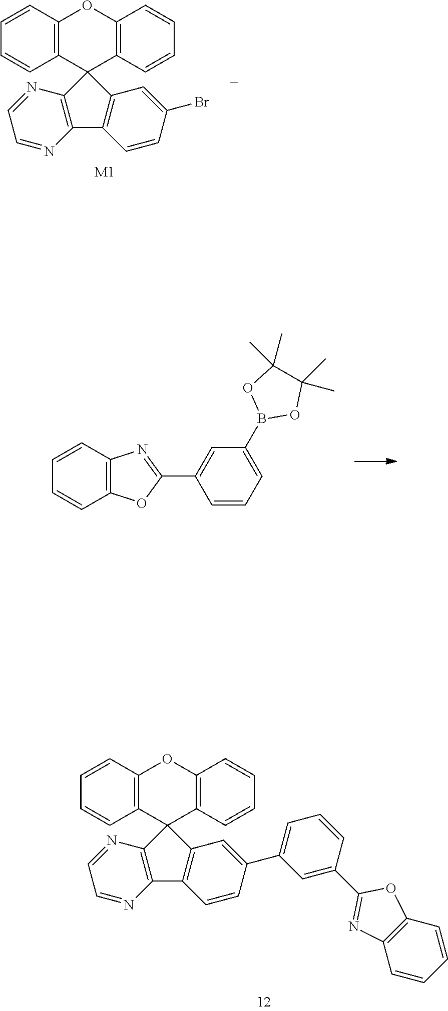

Synthesis of Compound 12: The synthesis of Compound 12 was similar to the synthesis of Compound 1, except that 2-(4,4,5,5-tetramethyl-1,3,2-dioxaborolan-2-yl)-1,10-phenanthroline was replaced by 2-(2-(4,4,5,5-tetramethyl-1,3,2-dioxaborolan-2-yl)phenyl)benzo[d]oxazole to obtain Compound 12. ¹H NMR (500 MHz, Chloroform) δ 8.62-8.46 (m, 3H), 8.16 (d, J=6.8 Hz, 2H), 8.03 (s, 1H), 7.72 (m, 3H), 7.61 (s, 1H), 7.35 (m, 4H), 7.26-7.12 (m, 5H), 7.00 (s, 2H).

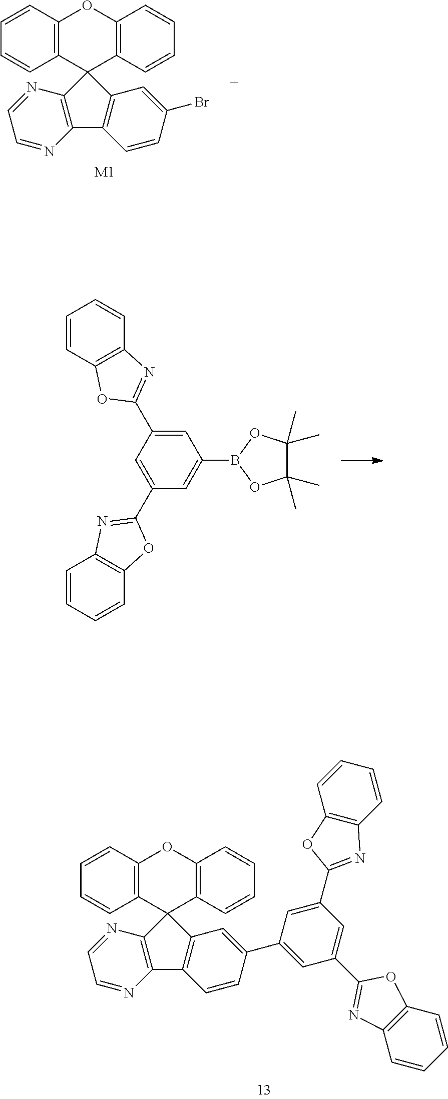

Synthesis of Compound 13: The synthesis of Compound 13 was similar to the synthesis of Compound 1, except that 2-(4,4,5,5-tetramethyl-1,3,2-dioxaborolan-2-yl)-1,10-phenanthroline was replaced by 2,2′-(5-(4,4,5,5-tetramethyl-1,3,2-dioxaborolan-2-yl)-1,3-phenyl)dibenzo[d]oxazole to obtain Compound 13. ¹H NMR (500 MHz, Chloroform) δ 8.62-8.46 (m, 3H), 8.33 (s, 1H), 8.24 (s, 2H), 8.03 (s, 1H), 7.74 (s, 4H), 7.35 (m, 6H), 7.25-7.12 (m, 5H), 7.00 (s, 2H).

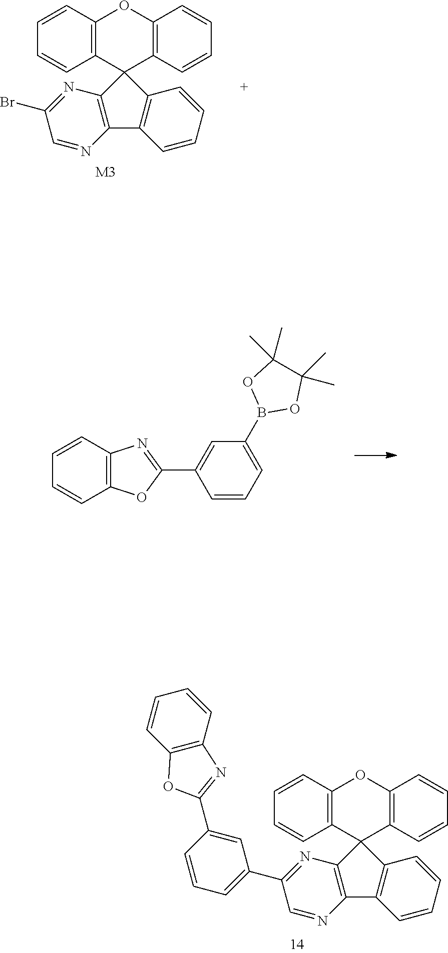

Synthesis of Compound 14: The synthesis of Compound 14 was similar to the synthesis of Compound 1, except that M1 was replaced by M3, and 2-(4,4,5,5-tetramethyl-1,3,2-dioxaborolan-2-yl)-1,10-phenanthroline was replaced by 2-(2-(4,4,5,5-tetramethyl-1,3,2-dioxaborolan-2-yl)phenyl)benzo[d]oxazole to obtain Compound 14. ¹H NMR (500 MHz, Chloroform) δ 8.52 (d, J=20.0 Hz, 2H), 8.45 (s, 1H), 8.25 (s, 1H), 8.18 (s, 1H), 7.74 (s, 3H), 7.47 (d, J=5.0 Hz, 2H), 7.35 (d. J=35.0 Hz, 5H), 7.18 (d, J=15.0 Hz, 4H), 7.00 (s, 2H).

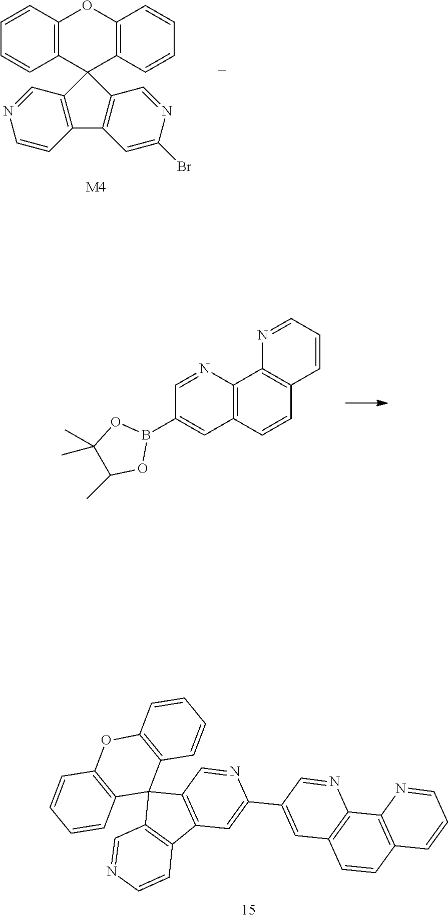 [SMILES: Brc1cc2c(cn1)C1(c3ccccc3Oc3ccccc31)c1cnccc1-2 ; "M4" ; CC1OB(c2cnc3c(ccc4cccnc43)c2)OC1(C)C ; c1ccc2c(c1)Oc1ccccc1C21c2cnccc2-c2cc(-c3cnc4c(ccc5cccnc54)c3)ncc21]

Synthesis of Compound 15: The synthesis of Compound 15 was similar to the synthesis of Compound 1, except that M1 was replaced by M4, and 2-(4,4,5,5-tetramethyl-1,3,2-dioxaborolan-2-yl)-1,10-phenanthroline was replaced by 3-(4,4,5,5-tetramethyl-1,3,2-dioxaborolan-2-yl)-1,10-phenanthroline to obtain Compound 15. ¹H NMR (500 MHz, Chloroform) δ 8.97 (d, J=20.0 Hz, 2H), 8.80 (d, J=4.0 Hz, 2H), 8.71 (s, 1H), 8.63 (s, 1H), 8.53 (s, 1H), 8.45 (s, 1H), 7.82 (s, 1H), 7.57 (d, J=5.0 Hz, 3H), 7.31 (s, 2H), 7.18 (m, 4H), 7.00 (s, 2H).

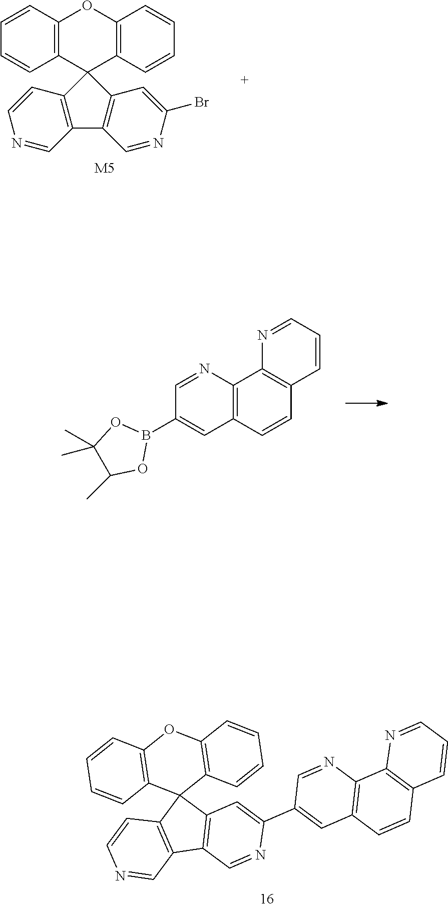

Synthesis of Compound 16: The synthesis of Compound 16 was similar to the synthesis of Compound 1, except that M1 was replaced by M5, and 2-(4,4,5,5-tetramethyl-1,3,2-dioxaborolan-2-yl)-1,10-phenanthroline was replaced by 3-(4,4,5,5-tetramethyl-1,3,2-dioxaborolan-2-yl)-1,10-phenanthroline to obtain Compound 16. ¹H NMR (500 MHz, Chloroform) δ 9.25 (d, J=7.2 Hz, 2H), 8.99 (s, 1H), 8.79 (d, J=5.3 Hz, 2H), 8.45 (s, 1H), 8.18 (d, J=20.0 Hz, 2H), 7.82 (s, 1H), 7.57 (d, J=5.0 Hz, 2H), 7.31 (s, 2H), 7.18 (m, 4H), 7.06 (s, 1H), 7.00 (s, 2H).

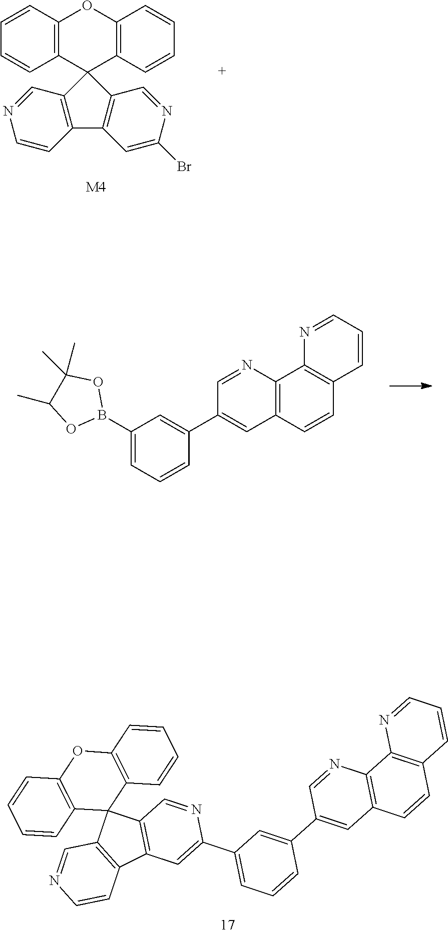 [SMILES: Brc1cc2c(cn1)C1(c3ccccc3Oc3ccccc31)c1cnccc1-2 ; "M4" ; CC1OB(c2cccc(-c3cnc4c(ccc5cccnc54)c3)c2)OC1(C)C ; c1cc(-c2cnc3c(ccc4cccnc43)c2)cc(-c2cc3c(cn2)C2(c4ccccc4Oc4ccccc42)c2cnccc2-3)c1]

Synthesis of Compound 17: The synthesis of Compound 17 was similar to the synthesis of Compound 1, except that M1 was replaced by M4, and 2-(4,4,5,5-tetramethyl-1,3,2-dioxaborolan-2-yl)-1,10-phenanthroline was replaced by 3-(3-(4,4,5,5-tetramethyl-1,3,2-dioxaborolan-2-yl)phenyl)-1,10-phenanthroline to obtain Compound 17. ¹H NMR (500 MHz, Chloroform) δ 8.95 (s, 1H), 8.80 (s, 1H), 8.63 (s, 1H), 8.52-8.38 (m, 4H), 8.34 (d, J=10.0 Hz, 2H), 8.26 (s, 1H), 7.80 (s, 1H), 7.70 (s, 1H), 7.65-7.49 (m, 4H), 7.31 (s, 2H), 7.18 (m, 4H), 7.00 (s, 2H).

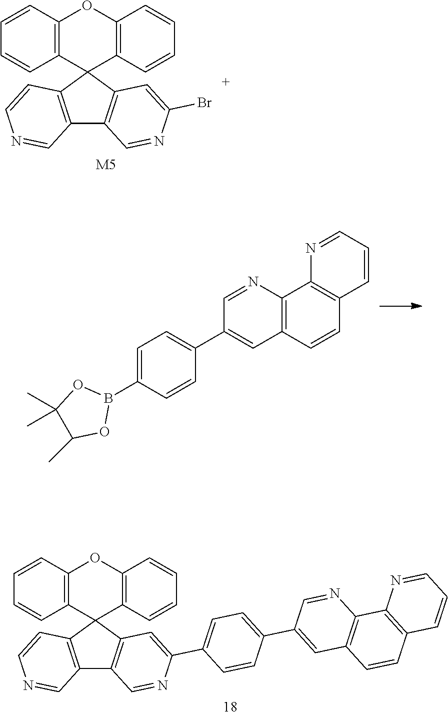

Synthesis of Compound 18: The synthesis of Compound 18 was similar to the synthesis of Compound 1, except that M1 was replaced by M5, and 2-(4,4,5,5-tetramethyl-1,3,2-dioxaborolan-2-yl)-1,10-phenanthroline was replaced by 3-(3-(4,4,5,5-tetramethyl-1,3,2-dioxaborolan-2-yl)phenyl)-1,10-phenanthroline to obtain Compound 18. ¹H NMR (500 MHz, Chloroform) δ 9.24 (s, 1H), 9.18 (s, 1H), 8.80 (s, 1H), 8.69 (s, 2H), 8.46 (d, J=5.8 Hz, 2H), 8.23 (m, 2H), 7.98 (s, 1H), 7.80 (s, 1H), 7.58 (m, 2H), 7.40-7.11 (m, 8H), 7.03 (m, 3H).

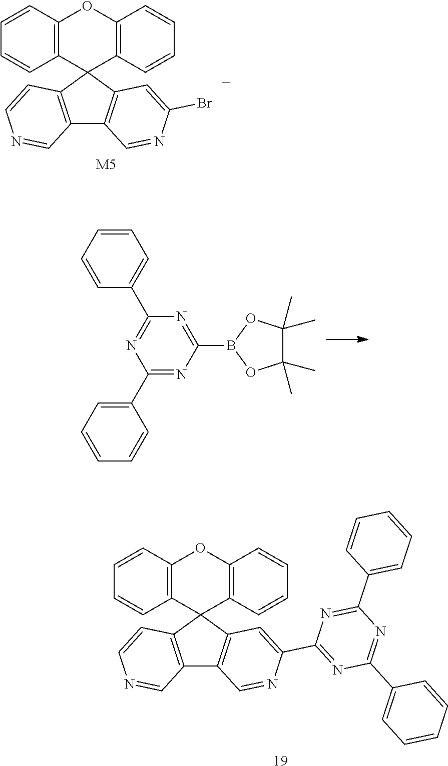

Synthesis of Compound 19: The synthesis of Compound 19 was similar to the synthesis of Compound 1, except that M1 was replaced by M5, and 2-(4,4,5,5-tetramethyl-1,3,2-dioxaborolan-2-yl)-1,10-phenanthroline was replaced by 2,4-diphenyl-6-(4,4,5,5-tetramethyl-1,3,2-dioxaborolan-2-yl)-1,3,5-triazine to obtain Compound 19. ¹H NMR (500 MHz, Chloroform) δ 9.29 (s, 1H), 9.24 (s, 1H), 8.36 (s, 4H), 8.18 (m, 2H), 7.50 (s, 6H), 7.31 (s, 2H), 7.18 (m, 4H), 7.06 (s, 1H), 7.00 (s, 2H).

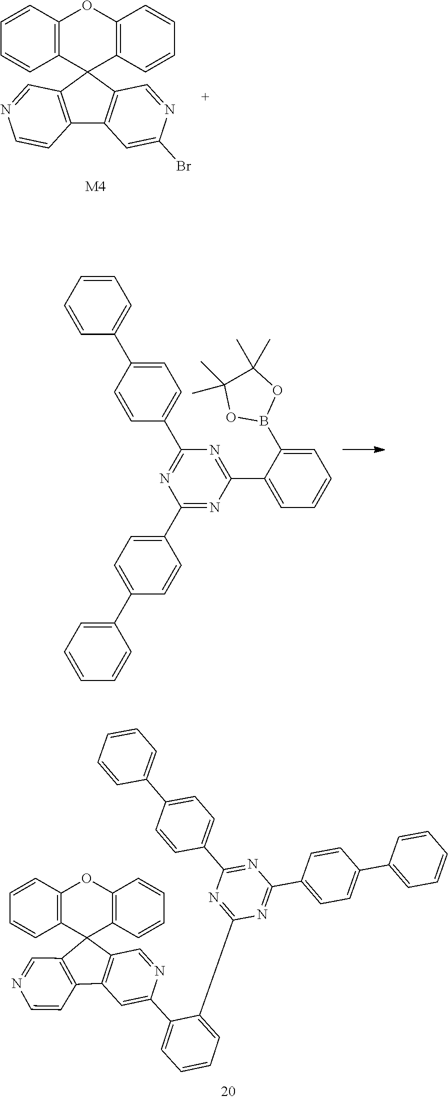 [SMILES: Brc1cc2c(cn1)C1(c3ccccc3Oc3ccccc31)c1cnccc1-2 ; "M4" ; CC1(C)OB(c2ccccc2-c2nc(-c3ccc(-c4ccccc4)cc3)nc(-c3ccc(-c4ccccc4)cc3)n2)OC1(C)C ; c1ccc(-c2ccc(-c3nc(-c4ccc(-c5ccccc5)cc4)nc(-c4ccccc4-c4cc5c(cn4)C4(c6ccccc6Oc6ccccc64)c4cnccc4-5)n3)cc2)cc1]

Synthesis of Compound 20: The synthesis of Compound 20 was similar to the synthesis of Compound 1, except that M1 was replaced by M4, and 2-(4,4,5,5-tetramethyl-1,3,2-dioxaborolan-2-yl)-1,10-phenanthroline was replaced by 2,4-bis([1,1′-diphenyl]-4-yl)-6-(2-(4,4,5,5-tetramethyl-1,3,2-dioxaborolan-2-yl)phenyl)-1,3,5-triazine, to obtain Compound 20. ¹H NMR (500 MHz, Chloroform) δ 8.95 (s, 1H), 8.63 (s, 1H), 8.49 (s, 1H), 8.35 (s. 2H), 7.96 (s, 5H), 7.75 (s, 4H), 7.66-7.12 (m, 19H), 7.00 (s, 2H).

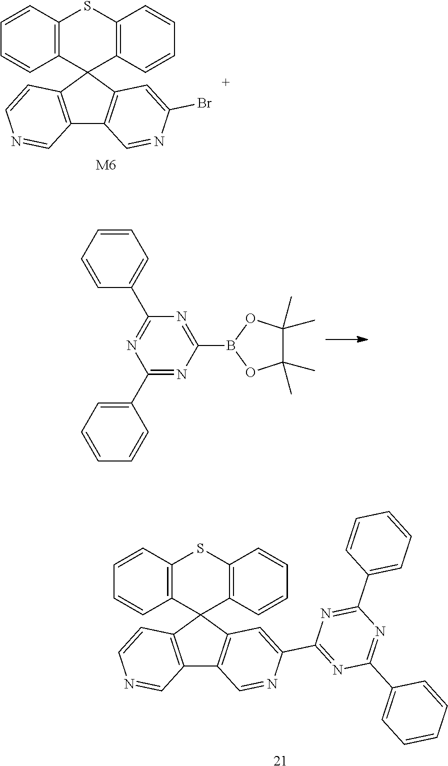

Synthesis of Compound 21: The synthesis of Compound 21 was similar to the synthesis of Compound 1, except that M1 was replaced by M6, and 2-(4,4,5,5-tetramethyl-1,3,2-dioxaborolan-2-yl)-1,10-phenanthroline was replaced by 2,4-diphenyl-6-(4,4,5,5-tetramethyl-1,3,2-dioxaborolan-2-yl)-1,3,5-triazine to obtain Compound 21. ¹H NMR (500 MHz, Chloroform) δ 9.25 (d, J=5.5 Hz, 2H), 8.36 (s, 4H), 8.18 (m, 2H), 7.69 (s, 2H), 7.50 (s, 6H), 7.33 (s, 2H), 7.10-6.95 (m, 5H).

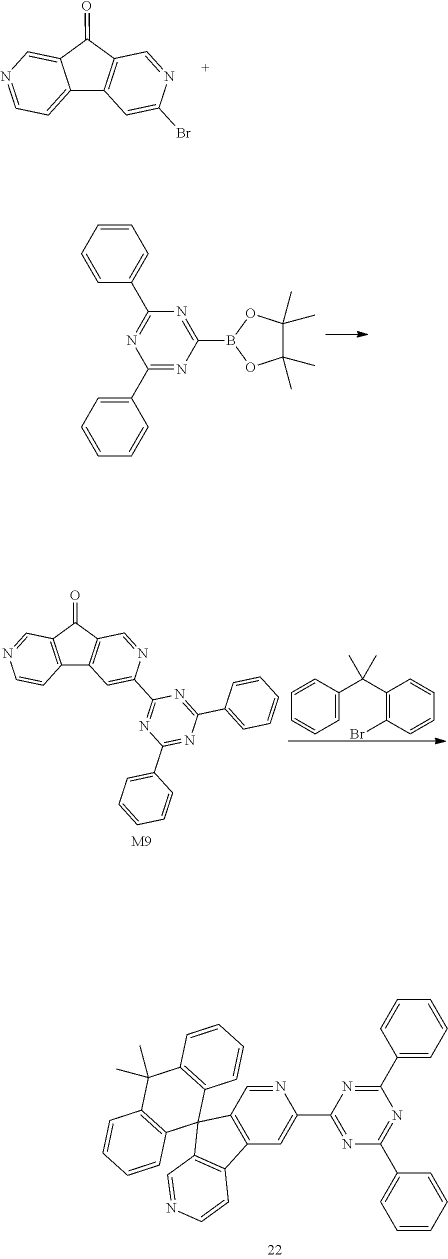

Synthesis of Intermediate M9: The synthesis of Intermediate M9 was similar to the synthesis of Intermediate M8, except that 7-bromo-9H-indolo[1,2-b]pyrazol-9-one was replaced by 3-bromo-9H-cyclopenta[1,2-c:4,3-c′]dipyridin-9-one, and 2-([1,1′-diphenyl]-4-yl)-4-phenyl-6-(3-(4,4,5,5-tetramethyl-1,3,2-dioxaborolan-2-yl)phenyl)-1,3,5-triazine was replaced by 2,4-diphenyl-6-(4,4,5,5-tetramethyl-1,3,2-dioxaborolan-2-yl)-1,3,5-triazine. ¹H NMR (500 MHz, Chloroform) δ 9.17 (s. 1H), 9.08 (s, 1H), 9.06 (s, 1H), 8.74 (s, 1H), 8.36 (s, 4H), 7.77 (s, 1H), 7.50 (s, 6H).

Synthesis of Compound 22: The synthesis of Compound 22 was similar to the synthesis of Compound 11, except that the Intermediate M8 was replaced by M9. ¹H NMR (500 MHz, Chloroform) δ 8.95 (s, 1H), 8.71 (s, 1H), 8.63 (s, 1H), 8.53 (s, 1H), 8.36 (s, 4H), 7.56 (s, 1H), 7.48 (m, 8H), 7.40 (s, 2H), 7.15 (s, 4H), 1.69 (s, 6H).

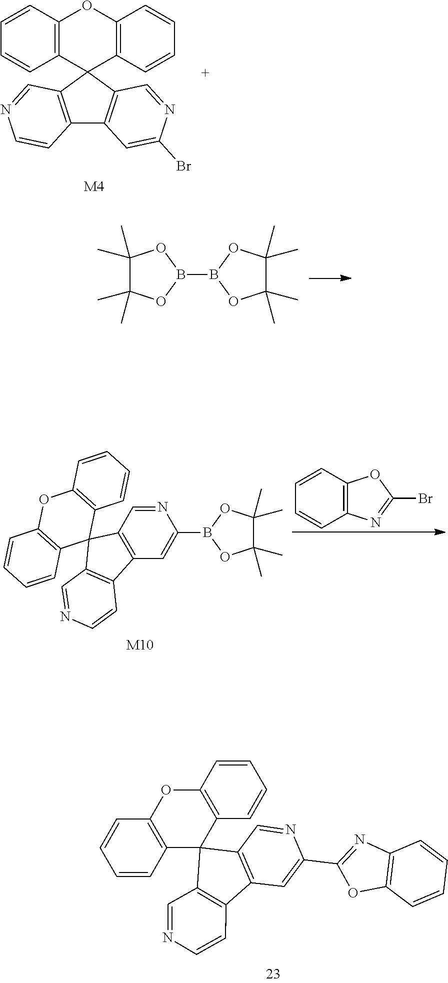

Synthesis of Intermediate M10: M4 (3.0 g, 7.2 mmol), bis(pinacolato)diboron (2.13 g, 8.40 mmol), Pd(dppf)Cl₂ (256 mg, 0.35 mmol), potassium acetate (2.06 g, 21.1 mmol) and 1,4-dioxane (35 mL) were added to a 125 mL round-bottomed flask, and heated to 100° C. for reflux reaction under argon protection for 12 h. After the reaction was completed, the reaction mixture was cooled to room temperature, evaporated under reduced pressure to remove 1,4-dioxane, and then separated and purified by silica gel column chromatography (petroleum ether:DCM=20:1) to obtain a light yellow powdery solid (2.48 g, 5.4 mmol). Yield: 75%, ¹H NMR (500 MHz, Chloroform) δ 8.95 (s, 1H), 8.95 (s, 1H), 8.63 (s, 1H), 8.63 (s, 1H), 8.52 (s, 1H), 8.52 (s, 1H), 7.63 (s, 1H), 7.63 (s, 1H), 7.56 (s, 1H), 7.31 (s, 2H), 7.18 (d, J=15.0 Hz, 4H), 7.00 (s, 2H), 1.14 (s, 12H).

Synthesis of Compound 23: Intermediate M10 (2.48 g, 5.4 mmol), 2-bromobenzo[d]oxazole (1.18 g, 6 mmol), Pd(PPh₃)₄ (0.58 g, 0.5 mmol), potassium carbonate (2.76 g, 20 mmol) and THF/H₂O (60 mL/12 mL) were added to a 200 mL pressure bottle, and heated under argon protection to 100° C. to perform pressurized reaction for 12 h. After the reaction was completed, the reaction mixture was cooled to room temperature and a large amount of solid precipitated. The reaction solution was filtered, and the filtered solid was washed with ethanol (100 mL) and dried to obtain a yellow powdery solid (1.95 g, 4.32 mmol) with a yield of 80%, ¹H NMR (500 MHz, Chloroform) δ 8.95 (s, 1H), 8.71 (s, 1H), 8.63 (s, 1H), 8.53 (s, 1H), 7.74 (s, 2H), 7.56 (s, 1H), 7.38 (s, 2H), 7.31 (s, 2H), 7.18 (d, J=15.0 Hz, 4H), 7.00 (s, 2H).

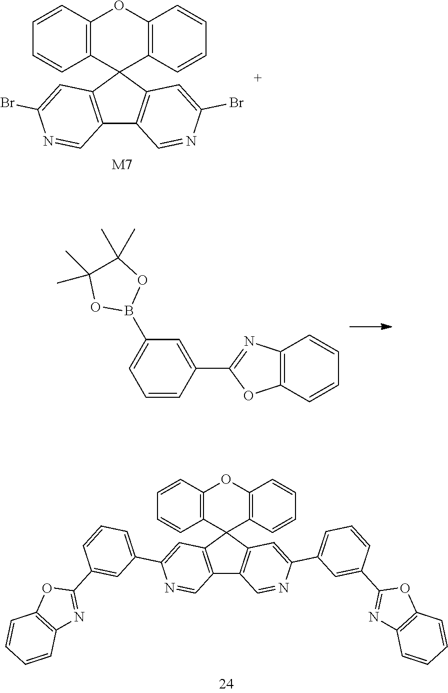 [SMILES: Brc1cc2c(cn1)-c1cnc(Br)cc1C21c2ccccc2Oc2ccccc21 ; "M7" ; CC1(C)OB(c2cccc(-c3nc4ccccc4o3)c2)OC1(C)C ; c1cc(-c2cc3c(cn2)-c2cnc(-c4cccc(-c5nc6ccccc6o5)c4)cc2C32c3ccccc3Oc3ccccc32)cc(-c2nc3ccccc3o2)c1]

Synthesis of Compound 24: The synthesis of Compound 24 was similar to the synthesis of Compound 1, except that M1 was replaced by M7, and 2-(4,4,5,5-tetramethyl-1,3,2-dioxaborolan-2-yl)-1,10-phenanthroline was replaced by 2-(2-(4,4,5,5-tetramethyl-1,3,2-dioxaborolan-2-yl)phenyl)benzo[d]oxazole to obtain Compound 24. ¹H NMR (500 MHz, Chloroform) δ 9.17 (s, 2H), 8.47 (s, 2H), 8.33 (s, 2H), 8.15 (s, 2H), 7.98 (s, 2H), 7.72 (m, 6H), 7.38 (s, 4H), 7.31 (s, 2H), 7.18 (m, 4H), 7.00 (s, 2H).

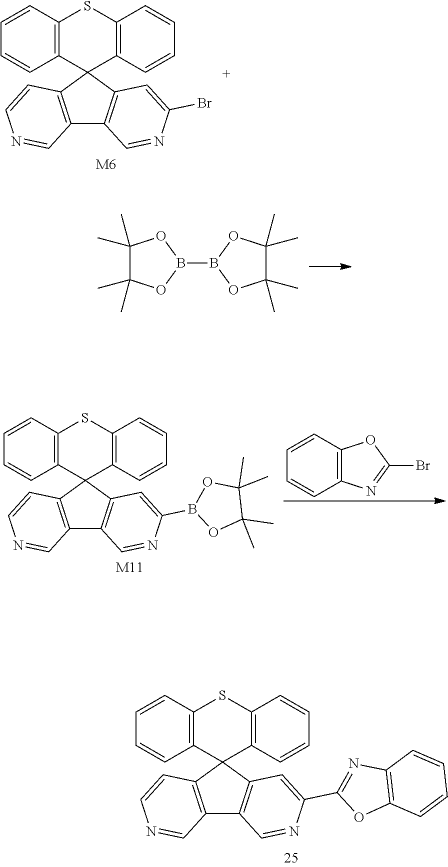

Synthesis of Intermediate M11: The synthesis of Intermediate M11 was similar to the synthesis of Intermediate M10, except that Intermediate M4 was replaced by M6. ¹H NMR (500 MHz, Chloroform) δ 9.24 (s, 1H), 8.89 (s, 1H), 8.20 (s, 1H), 7.69 (s, 2H), 7.42 (s, 1H), 7.33 (s, 2H), 7.12-6.95 (m, 5H), 1.14 (s, 12H).

Synthesis of Compound 25: The synthesis of Compound 25 was similar to the synthesis of Compound 23, except that Intermediate M10 was replaced by M11 ¹H NMR (500 MHz, Chloroform) δ 9.24 (s, 1H), 9.11 (s, 1H), 8.18 (d, J=20.0 Hz, 2H), 7.71 (m, 4H), 7.36 (m, 4H), 7.11-6.92 (m, 5H).

### 2. Physical and Chemical Properties of Compounds:

1. Using quantum chemistry simulation software at the B3LYP/6-31G* level, the Highest Occupied Molecular Orbital (HOMO) and the Lowest Unoccupied Molecular Orbital (LUMO) of the compound of the present disclosure were simulated respectively. The simulation calculations for HOMO, LUMO energy levels and Reorganization Energy (ROE) were conducted using DFT method and B3LYP functional. Some exemplary results were shown in Tables 1-1 to 1-2 below.

TABLE 1-1

| | | Reorganization | HOMO | LUMO |
|---|---|---|---|---|
| Material No. | Molecular structure | Energy (eV) | (eV) | (eV) |
| Compound 1 | 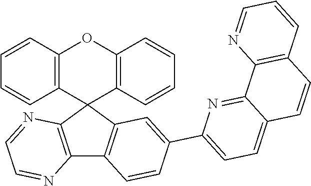 [SMILES: c1ccc2c(c1)Oc1ccccc1C21c2cc(-c3ccc4ccc5cccnc5c4n3)ccc2-c2nccnc21] | 0.33 | 5.90 | 1.77 |
| Compound 2 | 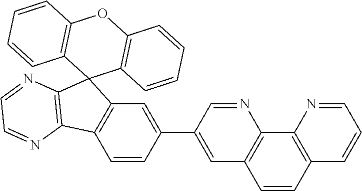 [SMILES: c1ccc2c(c1)Oc1ccccc1C21c2cc(-c3cnc4c(ccc5cccnc54)c3)ccc2-c2nccnc21] | 0.34 | 5.96 | 1.78 |
| Compound 3 | 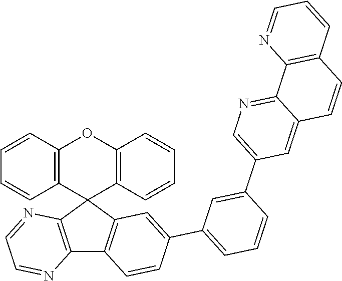 [SMILES: c1cc(-c2ccc3c(c2)C2(c4ccccc4Oc4ccccc42)c2nccnc2-3)cc(-c2cnc3c(ccc4cccnc43)c2)c1] | 0.29 | 5.66 | 1.90 |
| Compound 4 | 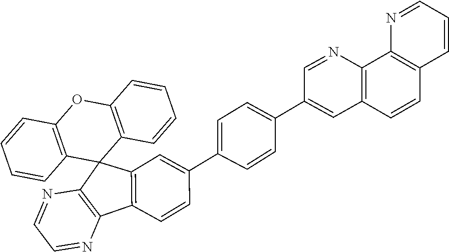 [SMILES: c1ccc2c(c1)Oc1ccccc1C21c2cc(-c3ccc(-c4cnc5c(ccc6cccnc65)c4)cc3)ccc2-c2nccnc21] | 0.27 | 5.65 | 1.91 |
| Compound 5 | 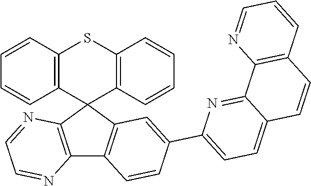 [SMILES: c1ccc2c(c1)Sc1ccccc1C21c2cc(-c3ccc4ccc5cccnc5c4n3)ccc2-c2nccnc21] | 0.34 | 5.90 | 1.79 |
| Compound 6 | 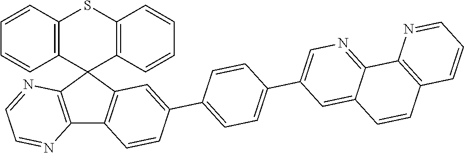 [SMILES: c1ccc2c(c1)Sc1ccccc1C21c2cc(-c3ccc(-c4cnc5c(ccc6cccnc65)c4)cc3)ccc2-c2nccnc21] | 0.29 | 5.60 | 1.89 |
| Compound 7 | 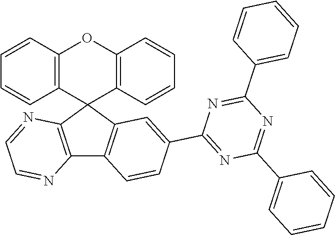 [SMILES: c1ccc(-c2nc(-c3ccccc3)nc(-c3ccc4c(c3)C3(c5ccccc5Oc5ccccc53)c3nccnc3-4)n2)cc1] | 0.32 | 5.7 | 1.87 |
| Compound 8 | 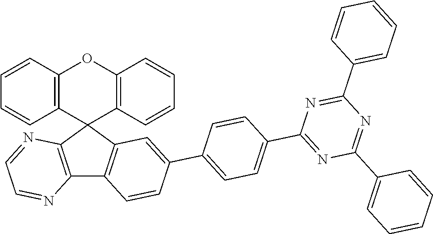 [SMILES: c1ccc(-c2nc(-c3ccccc3)nc(-c3ccc(-c4ccc5c(c4)C4(c6ccccc6Oc6ccccc64)c4nccnc4-5)cc3)n2)cc1] | 0.28 | 5.75 | 2.08 |
| Compound 9 | 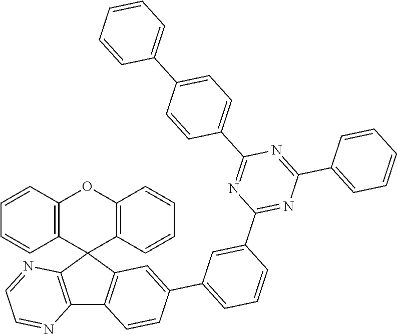 [SMILES: c1ccc(-c2ccc(-c3nc(-c4ccccc4)nc(-c4cccc(-c5ccc6c(c5)C5(c7ccccc7Oc7ccccc75)c5nccnc5-6)c4)n3)cc2)cc1] | 0.32 | 5.8 | 2.09 |
| Compound 10 | 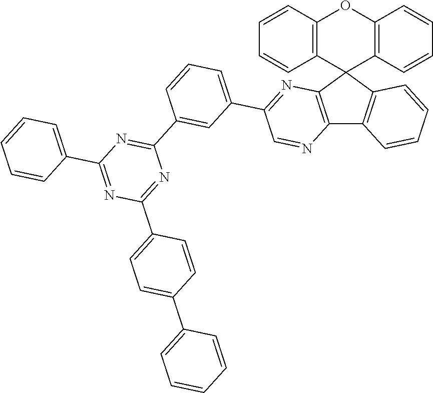 [SMILES: c1ccc(-c2ccc(-c3nc(-c4ccccc4)nc(-c4cccc(-c5cnc6c(n5)C5(c7ccccc7Oc7ccccc75)c5ccccc5-6)c4)n3)cc2)cc1] | 0.39 | 5.48 | 2.1 |
| Compound 11 | 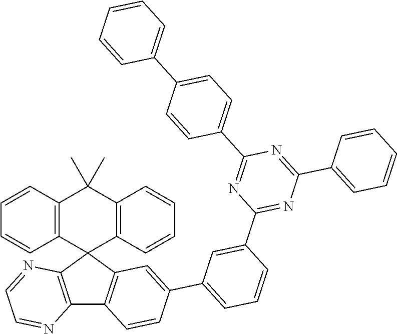 [SMILES: CC1(C)c2ccccc2C2(c3cc(-c4cccc(-c5nc(-c6ccccc6)nc(-c6ccc(-c7ccccc7)cc6)n5)c4)ccc3-c3nccnc32)c2ccccc21] | 0.34 | 5.82 | 2.01 |
| Compound 12 | 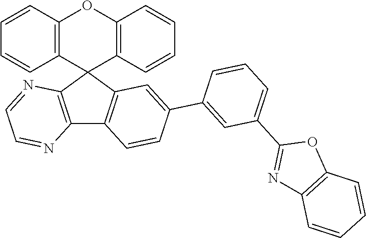 [SMILES: c1cc(-c2ccc3c(c2)C2(c4ccccc4Oc4ccccc42)c2nccnc2-3)cc(-c2nc3ccccc3o2)c1] | 0.26 | 5.44 | 2.06 |
| Compound 13 | 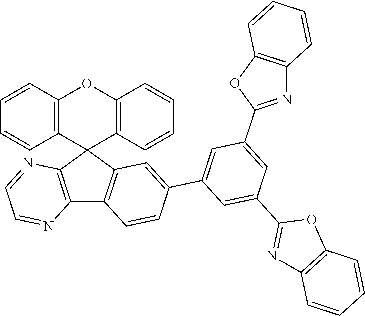 [SMILES: c1ccc2c(c1)Oc1ccccc1C21c2cc(-c3cc(-c4nc5ccccc5o4)cc(-c4nc5ccccc5o4)c3)ccc2-c2nccnc21] | 0.27 | 5.69 | 2.14 |
| Compound 14 | 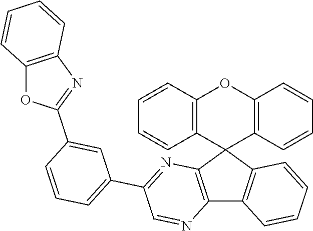 [SMILES: c1cc(-c2cnc3c(n2)C2(c4ccccc4Oc4ccccc42)c2ccccc2-3)cc(-c2nc3ccccc3o2)c1] | 0.36 | 5.40 | 2.05 |

TABLE 1-2

| | | Reorganization | HOMO | LUMO |
|---|---|---|---|---|
| Material No. | Molecular structure | Energy (eV) | (eV) | (eV) |
| Compound 15 | 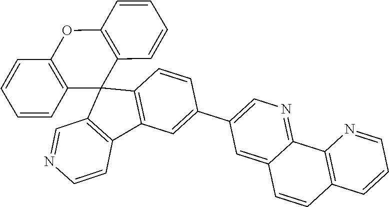 [SMILES: c1ccc2c(c1)Oc1ccccc1C21c2ccc(-c3cnc4c(ccc5cccnc54)c3)cc2-c2ccncc21] | 0.32 | 5.65 | 1.91 |
| Compound 16 | 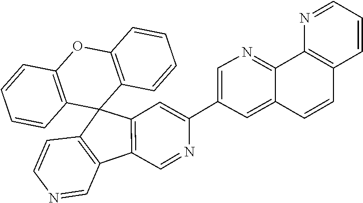 [SMILES: c1ccc2c(c1)Oc1ccccc1C21c2ccncc2-c2cnc(-c3cnc4c(ccc5cccnc54)c3)cc21] | 0.32 | 5.7 | 1.87 |
| Compound 17 | 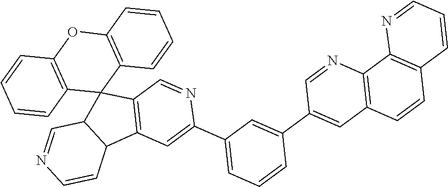 [SMILES: C1=CC2c3cc(-c4cccc(-c5cnc6c(ccc7cccnc76)c5)c4)ncc3C3(c4ccccc4Oc4ccccc43)C2C=N1] | 0.30 | 5.65 | 1.95 |
| Compound 18 | 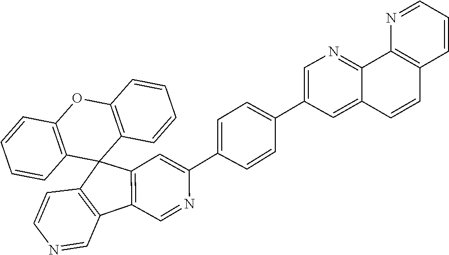 [SMILES: c1ccc2c(c1)Oc1ccccc1C21c2ccncc2-c2cnc(-c3ccc(-c4cnc5c(ccc6cccnc65)c4)cc3)cc21] | 0.29 | 5.8 | 2.09 |
| Compound 19 | 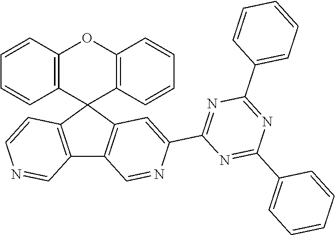 [SMILES: c1ccc(-c2nc(-c3ccccc3)nc(-c3cc4c(cn3)-c3cnccc3C43c4ccccc4Oc4ccccc43)n2)cc1] | 0.26 | 5.58 | 2.1 |
| Compound 20 | 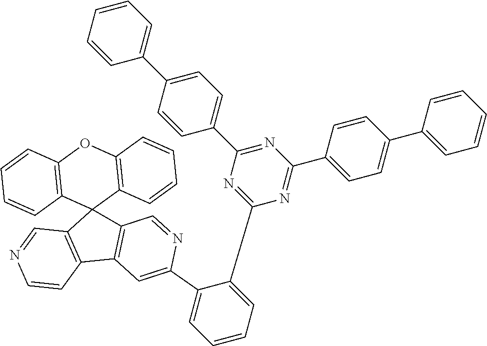 [SMILES: c1ccc(-c2ccc(-c3nc(-c4ccc(-c5ccccc5)cc4)nc(-c4ccccc4-c4cc5c(cn4)C4(c6ccccc6Oc6ccccc64)c4cnccc4-5)n3)cc2)cc1] | 0.24 | 5.60 | 2.08 |
| Compound 21 | 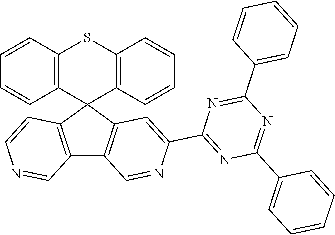 [SMILES: c1ccc(-c2nc(-c3ccccc3)nc(-c3cc4c(cn3)-c3cnccc3C43c4ccccc4Sc4ccccc43)n2)cc1] | 0.27 | 5.56 | 2.11 |
| Compound 22 | 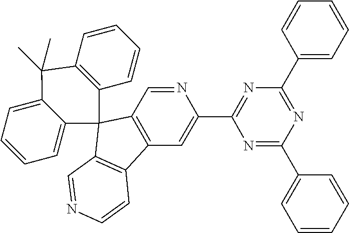 [SMILES: CC1(C)c2ccccc2C2(c3cnccc3-c3cc(-c4nc(-c5ccccc5)nc(-c5ccccc5)n4)ncc32)c2ccccc21] | 0.27 | 5.96 | 1.78 |
| Compound 23 | 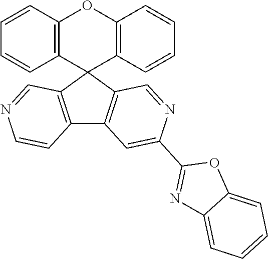 [SMILES: c1ccc2c(c1)Oc1ccccc1C21c2cnccc2-c2cc(-c3nc4ccccc4o3)ncc21] | 0.25 | 5.75 | 1.96 |
| Compound 24 | 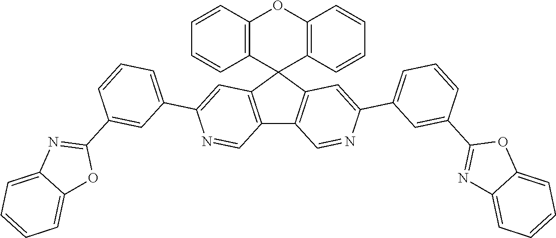 [SMILES: c1cc(-c2cc3c(cn2)-c2cnc(-c4cccc(-c5nc6ccccc6o5)c4)cc2C32c3ccccc3Oc3ccccc32)cc(-c2nc3ccccc3o2)c1] | 0.27 | 5.8 | 1.92 |
| Compound 25 | 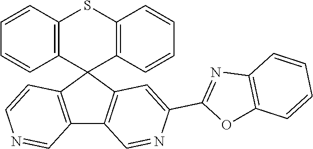 [SMILES: c1ccc2c(c1)Sc1ccccc1C21c2ccncc2-c2cnc(-c3nc4ccccc4o3)cc21] | 0.31 | 5.7 | 1.99 |

2. The glass transition temperature (Tg) determines the thermal stability of the electron transport material during evaporation. The higher the Tg, the better the thermal stability of the electron transport material. The measuring instrument used was a DSC differential scanning calorimeter, with a nitrogen atmosphere, a heating rate of 10° C./min, and a temperature range of 50 to 300° C. The calculation for T1 energy level was conducted using TDDFT method and B3LYP functional. Some exemplary results of the compounds of the present disclosure were shown in Tables 2-1 to 2-2 below.

TABLE 2-1

| | | Tg | T1 |
|---|---|---|---|
| Material No. | Molecular structure | ° C. | eV |
| Compound 1 | 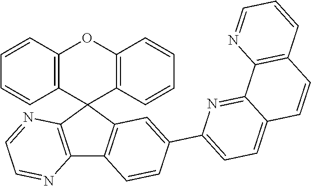 [SMILES: c1ccc2c(c1)Oc1ccccc1C21c2cc(-c3ccc4ccc5cccnc5c4n3)ccc2-c2nccnc21] | 123.952 | 2.54 |
| Compound 2 | 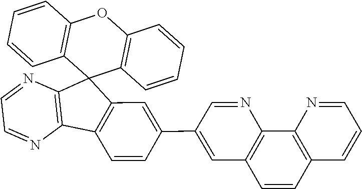 [SMILES: c1ccc2c(c1)Oc1ccccc1C21c2cc(-c3cnc4c(ccc5cccnc54)c3)ccc2-c2nccnc21] | 122.665 | 2.57 |
| Compound 3 | 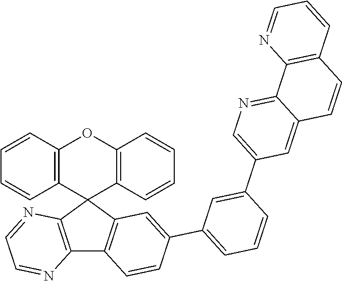 [SMILES: c1cc(-c2ccc3c(c2)C2(c4ccccc4Oc4ccccc42)c2nccnc2-3)cc(-c2cnc3c(ccc4cccnc43)c2)c1] | 164.12 | 2.42 |
| Compound 4 | 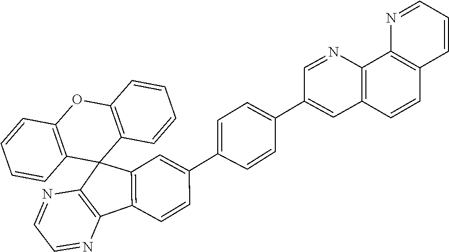 [SMILES: c1ccc2c(c1)Oc1ccccc1C21c2cc(-c3ccc(-c4cnc5c(ccc6cccnc65)c4)cc3)ccc2-c2nccnc21] | 166.275 | 2.54 |
| Compound 5 | 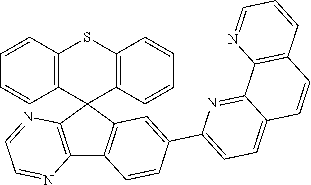 [SMILES: c1ccc2c(c1)Sc1ccccc1C21c2cc(-c3ccc4ccc5cccnc5c4n3)ccc2-c2nccnc21] | 120.281 | 2.58 |
| Compound 6 | 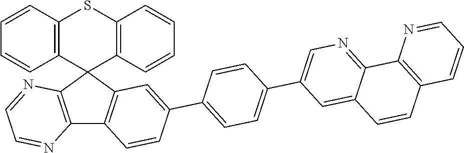 [SMILES: c1ccc2c(c1)Sc1ccccc1C21c2cc(-c3ccc(-c4cnc5c(ccc6cccnc65)c4)cc3)ccc2-c2nccnc21] | 164.256 | 2.54 |
| Compound 7 | 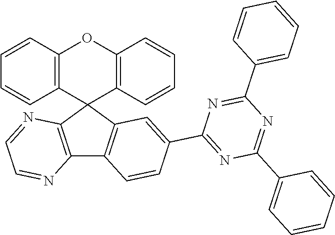 [SMILES: c1ccc(-c2nc(-c3ccccc3)nc(-c3ccc4c(c3)C3(c5ccccc5Oc5ccccc53)c3nccnc3-4)n2)cc1] | 145.641 | 2.38 |
| Compound 8 | 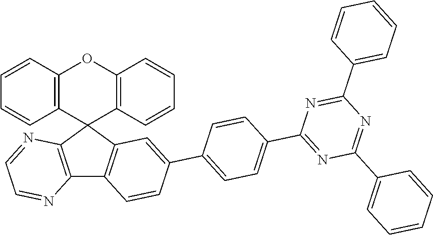 [SMILES: c1ccc(-c2nc(-c3ccccc3)nc(-c3ccc(-c4ccc5c(c4)C4(c6ccccc6Oc6ccccc64)c4nccnc4-5)cc3)n2)cc1] | 156.892 | 2.34 |
| Compound 9 | 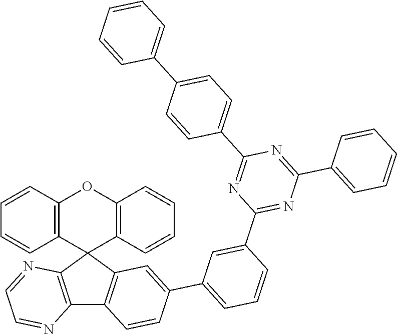 [SMILES: c1ccc(-c2ccc(-c3nc(-c4ccccc4)nc(-c4cccc(-c5ccc6c(c5)C5(c7ccccc7Oc7ccccc75)c5nccnc5-6)c4)n3)cc2)cc1] | 155.489 | 2.33 |
| Compound 10 | 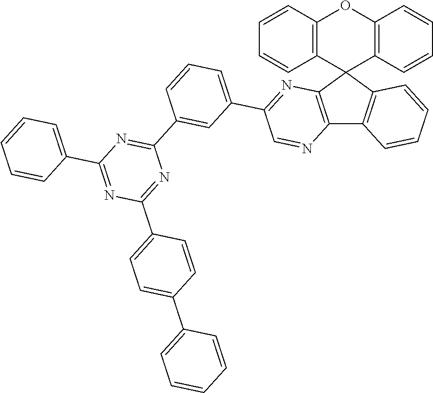 [SMILES: c1ccc(-c2ccc(-c3nc(-c4ccccc4)nc(-c4cccc(-c5cnc6c(n5)C5(c7ccccc7Oc7ccccc75)c5ccccc5-6)c4)n3)cc2)cc1] | 159.780 | 2.33 |
| Compound 11 | 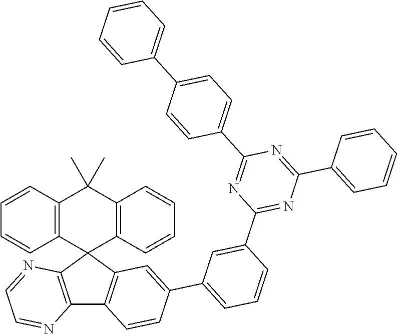 [SMILES: CC1(C)c2ccccc2C2(c3cc(-c4cccc(-c5nc(-c6ccccc6)nc(-c6ccc(-c7ccccc7)cc6)n5)c4)ccc3-c3nccnc32)c2ccccc21] | 150.249 | 2.34 |
| Compound 12 | 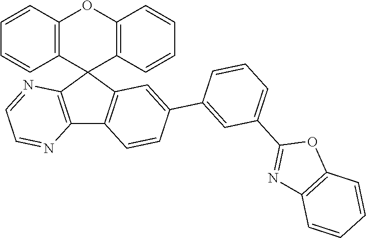 [SMILES: c1cc(-c2ccc3c(c2)C2(c4ccccc4Oc4ccccc42)c2nccnc2-3)cc(-c2nc3ccccc3o2)c1] | 122.312 | 2.56 |
| Compound 13 | 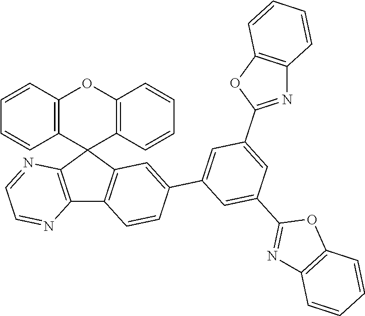 [SMILES: c1ccc2c(c1)Oc1ccccc1C21c2cc(-c3cc(-c4nc5ccccc5o4)cc(-c4nc5ccccc5o4)c3)ccc2-c2nccnc21] | 125.425 | 2.54 |
| Compound 14 | 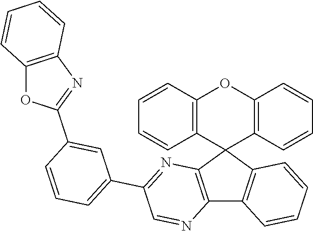 [SMILES: c1cc(-c2cnc3c(n2)C2(c4ccccc4Oc4ccccc42)c2ccccc2-3)cc(-c2nc3ccccc3o2)c1] | 127.842 | 2.56 |

TABLE 2-2

| Material No. | Molecular structure | Tg | T1 |
|---|---|---|---|
| Compound 15 | 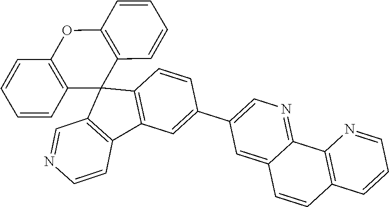 [SMILES: c1ccc2c(c1)Oc1ccccc1C21c2ccc(-c3cnc4c(ccc5cccnc54)c3)cc2-c2ccncc21] | 124.522 | 2.55 |
| Compound 16 | 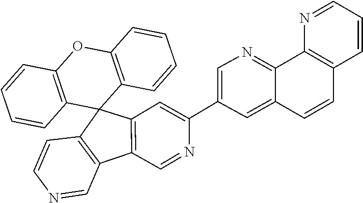 [SMILES: c1ccc2c(c1)Oc1ccccc1C21c2ccncc2-c2cnc(-c3cnc4c(ccc5cccnc54)c3)cc21] | 127.251 | 2.54 |
| Compound 17 | 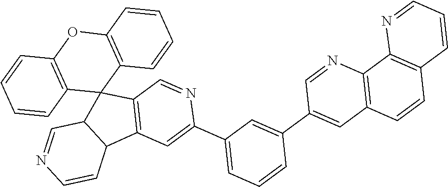 [SMILES: C1=CC2c3cc(-c4cccc(-c5cnc6c(ccc7cccnc76)c5)c4)ncc3C3(c4ccccc4Oc4ccccc43)C2C=N1] | 165.774 | 2.42 |
| Compound 18 | 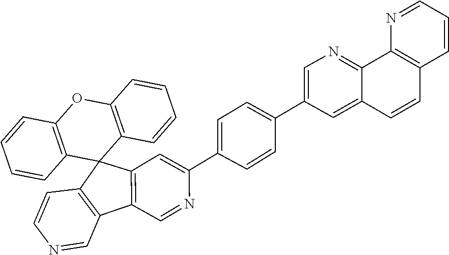 [SMILES: c1ccc2c(c1)Oc1ccccc1C21c2ccncc2-c2cnc(-c3ccc(-c4cnc5c(ccc6cccnc65)c4)cc3)cc21] | 166.881 | 2.58 |
| Compound 19 | 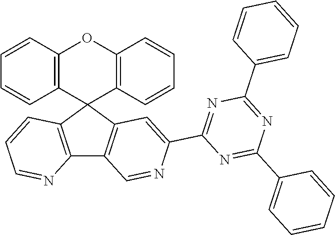 [SMILES: c1ccc(-c2nc(-c3ccccc3)nc(-c3cc4c(cn3)-c3ncccc3C43c4ccccc4Oc4ccccc43)n2)cc1] | 150.159 | 2.36 |
| Compound 20 | 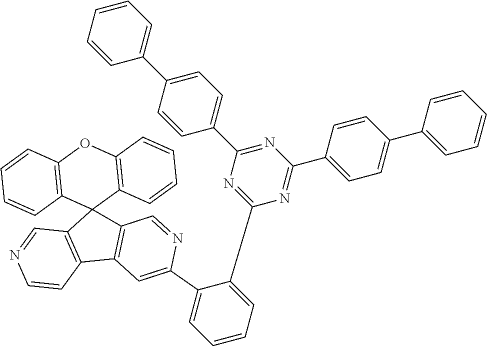 [SMILES: c1ccc(-c2ccc(-c3nc(-c4ccc(-c5ccccc5)cc4)nc(-c4ccccc4-c4cc5c(cn4)C4(c6ccccc6Oc6ccccc64)c4cnccc4-5)n3)cc2)cc1] | 162.347 | 2.30 |
| Compound 21 | 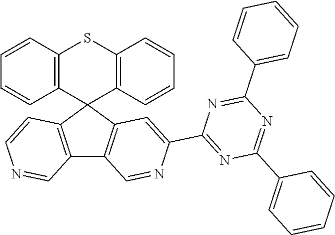 [SMILES: c1ccc(-c2nc(-c3ccccc3)nc(-c3cc4c(cn3)-c3cnccc3C43c4ccccc4Sc4ccccc43)n2)cc1] | 152.426 | 2.34 |
| Compound 22 | 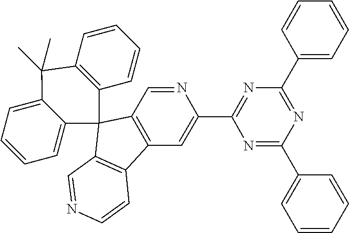 [SMILES: CC1(C)c2ccccc2C2(c3cnccc3-c3cc(-c4nc(-c5ccccc5)nc(-c5ccccc5)n4)ncc32)c2ccccc21] | 144.021 | 2.35 |
| Compound 23 | 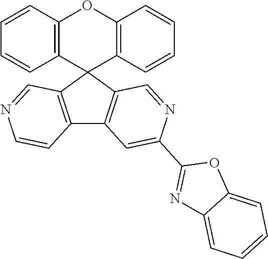 [SMILES: c1ccc2c(c1)Oc1ccccc1C21c2cnccc2-c2cc(-c3nc4ccccc4o3)ncc21] | 124.102 | 2.30 |
| Compound 24 | 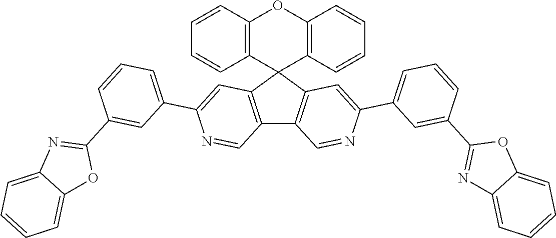 [SMILES: c1cc(-c2cc3c(cn2)-c2cnc(-c4cccc(-c5nc6ccccc6o5)c4)cc2C32c3ccccc3Oc3ccccc32)cc(-c2nc3ccccc3o2)c1] | 128.691 | 2.57 |
| Compound 25 | 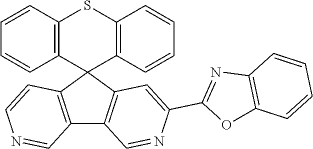 [SMILES: c1ccc2c(c1)Sc1ccccc1C21c2ccncc2-c2cnc(-c3nc4ccccc4o3)cc21] | 125.412 | 2.55 |

### Experimental Example 1

Materials for the functional layer of the luminescent device in this experimental example: HAT-CN was the hole injection layer, NPB was the hole transport layer, TCTA was the electron blocking layer, DPEPO was the hole blocking layer, ADN was the blue light host, and DPAVBi was the blue light dopant (these materials were all commercially available), and the electron transport layer material employed the compound provided by the present disclosure; the specific chemical structural formulas were as follows:

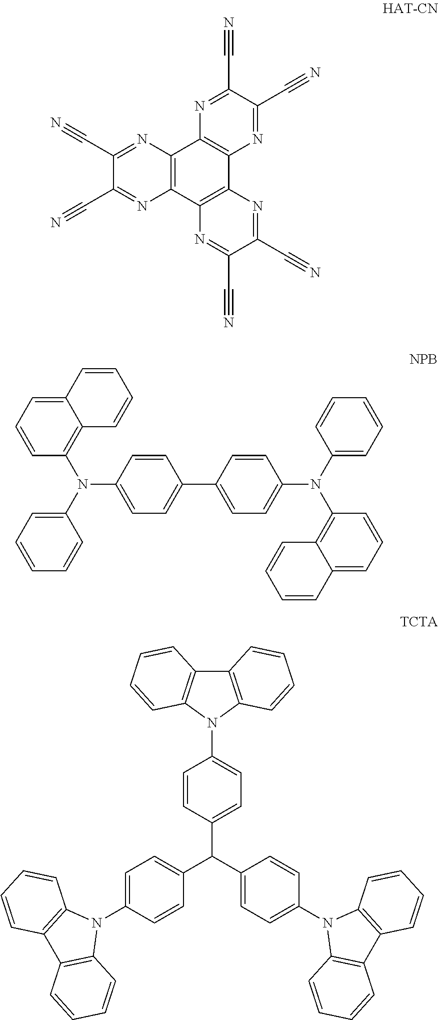 [SMILES: N#Cc1nc2c3nc(C#N)c(C#N)nc3c3nc(C#N)c(C#N)nc3c2nc1C#N ; c1ccc(N(c2ccc(-c3ccc(N(c4ccccc4)c4cccc5ccccc45)cc3)cc2)c2cccc3ccccc23)cc1 ; c1ccc2c(c1)c1ccccc1n2-c1ccc(C(c2ccc(-n3c4ccccc4c4ccccc43)cc2)c2ccc(-n3c4ccccc4c4ccccc43)cc2)cc1]

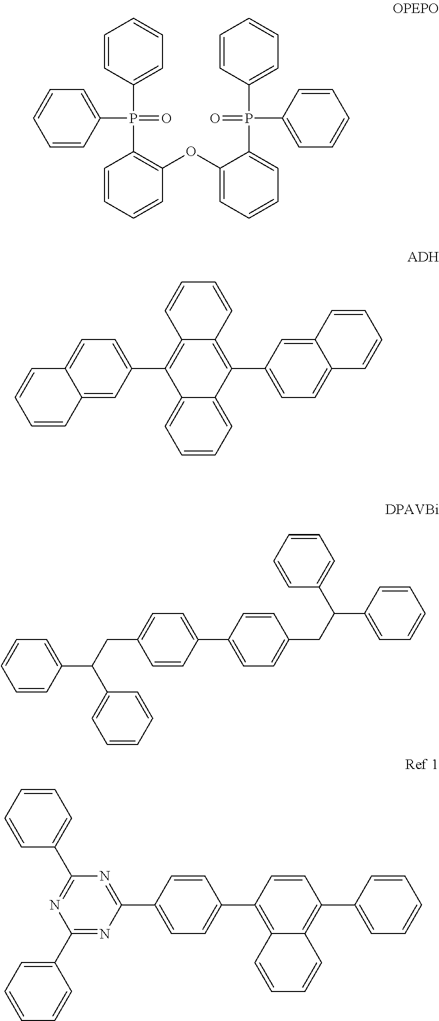

The preparation process of the luminescent device in this experimental example was as follows:

A substrate (glass plate) provided with a first electrode 1 (ITO) was ultrasonically treated in a detergent, rinsed in deionized water, ultrasonically degreased in an acetone-ethanol mixed solvent, and baked in a clean environment until moisture was removed completely.

The glass plate with ITO was placed in a vacuum chamber, where the vacuum was pulled to 1×10⁻⁵ to 1×10⁻⁶, and a hole injection material was vacuum evaporated on the side of the ITO far away from the glass plate to form a hole injection layer.

A hole transport material was evaporated on the side of the hole injection layer far away from ITO to form a hole transport layer.

An electron blocking material was vacuum evaporated on the side of the hole transport layer far away from the hole injection layer to form an electron blocking layer.

A luminescent material was vacuum evaporated on the side of the electron blocking layer far away from the hole transport layer to form a light emitting layer. The luminescent material comprised a host material and a guest material, using a multi-source co-evaporation method with a weight ratio of the host material to the guest material at 95:5.

A hole blocking material was vacuum evaporated on the side of the light emitting layer far away from the electron blocking layer to form a hole blocking layer.

An electron transport material was vacuum evaporated on the side of the hole blocking layer far away from the light emitting layer to form an electron transport layer.

On the side of the electron transport layer far away from the hole blocking layer, an inorganic substance (LiF) with a thickness of 1 nm was vacuum evaporated as an electron injection material to form an electron injection layer.

An Al layer was vacuum evaporated on the side of the electron injection layer far away from the electron transport layer as a cathode.

The device structure of this experimental example was: Substrate/ITO/HAT-CN(20 nm)/NPB(50 nm)/TCTA(6 nm)/ADN+5% DPAVBi(20 nm)/DPEPO(10 nm)/Compound 1 (40 nm)/LiF(1 nm)/Al(100 nm); and Compound 1 served as the electron transport layer.

### Experimental Examples 2 to 25

The differences from Experimental Example 1 were that the electron transport layers used the above-mentioned Compounds 2 to 25 respectively instead of Compound 1 in Experimental Example 1. Other steps were the same as those in Experimental Example 1.

### Comparative Example

The difference from Experimental Example 1 was that the electron transport layer used Compound Ref. 1 instead of Compound 1 in Experimental Example 1, and other steps were the same as those in Experimental Example 1.

Performance tests were conducted on the electroluminescent devices prepared in the above Experimental Examples 1 to 25 and Comparative Example. The test method was as follows: the prepared devices were subjected to luminescence performance tests (turn-on voltage, brightness, external quantum efficiency, lifespan) under the condition of a current density of 15 mA/cm². The results were shown in Tables 3-1 to 3-2 below.

TABLE 3-1

| | | | | External | |
|---|---|---|---|---|---|
| | Electron | Turn-on | | quantum | |
| | transport layer | voltage | Brightness | efficiency | Lifespan |
| Luminescent device | material No. | (V) | (Cd/m²) | (%) | (LT95) |
| Comparative Example | Ref. 1 | 100% | 100% | 100% | 100% |
| Experimental Example 1 | Compound 1 | 96% | 115% | 110% | 120% |
| Experimental Example 2 | Compound 2 | 94% | 120% | 114% | 110% |
| Experimental Example 3 | Compound 3 | 90% | 132% | 118% | 130% |
| Experimental Example 4 | Compound 4 | 98% | 106% | 101% | 113% |
| Experimental Example 5 | Compound 5 | 92% | 126% | 120% | 128% |
| Experimental Example 6 | Compound 6 | 95% | 117% | 126% | 117% |
| Experimental Example 7 | Compound 7 | 99% | 105% | 100% | 120% |
| Experimental Example 8 | Compound 8 | 97% | 110% | 117% | 111% |
| Experimental Example 9 | Compound 9 | 92% | 122% | 108% | 120% |
| Experimental Example 10 | Compound 10 | 94% | 116% | 110% | 128% |
| Experimental Example 11 | Compound 11 | 96% | 113% | 121% | 108% |
| Experimental Example 12 | Compound 12 | 97% | 107% | 116% | 107% |
| Experimental Example 13 | Compound 13 | 99% | 100% | 107% | 121% |
| Experimental Example 14 | Compound 14 | 98% | 103% | 111% | 118% |

TABLE 3-2

| | | | | External | |
|---|---|---|---|---|---|
| | Electron | Turn-on | | quantum | |
| | transport layer | voltage | Brightness | efficiency | Lifespan |
| Luminescent device | material No. | (V) | (Cd/m²) | (%) | (LT95) |
| Comparative Example | Ref. 1 | 100% | 100% | 100% | 100% |
| Experimental Example 15 | Compound 15 | 104% | 97% | 104% | 112% |
| Experimental Example 16 | Compound 16 | 98% | 105% | 111% | 116% |
| Experimental Example 17 | Compound 17 | 92% | 130% | 124% | 130% |
| Experimental Example 18 | Compound 18 | 98% | 104% | 100% | 110% |
| Experimental Example 19 | Compound 19 | 102% | 102% | 102% | 110% |
| Experimental Example 20 | Compound 20 | 96% | 116% | 111% | 123% |
| Experimental Example 21 | Compound 21 | 95% | 102% | 100% | 103% |
| Experimental Example 22 | Compound 22 | 95% | 116% | 105% | 118% |
| Experimental Example 23 | Compound 23 | 91% | 117% | 106% | 111% |
| Experimental Example 24 | Compound 24 | 101% | 100% | 105% | 111% |
| Experimental Example 25 | Compound 25 | 98% | 103% | 112% | 108% |

It can be seen from the exemplary results in the above Tables 3-1 to 3-2 that the compounds of the present disclosure are electron transport materials with good device performance, the OLED devices utilizing them as the electron transport layer all showed high efficiency, low driving voltage and long lifespan electroluminescent performance.

1. A compound represented by Formula O, or a stereoisomer or tautomer thereof, 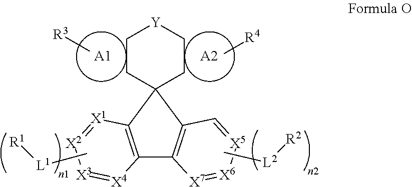
wherein:
X¹, X², X³, and X⁴ are each independently selected from CH and N, and at least one of X¹, X², X³ and X⁴ is N, and X³ and X⁴ are not N at the same time;
X⁵, X⁶, and X⁷ are each independently selected from CH and N, and X⁶ and X⁷ are not N at the same time;
Ring A1 and Ring A2 are each independently selected from C₆-C₁₂ aromatic rings;
Y is selected from —O—, —S—, —CR^{a}R^{b}—;
R^{a} and R^{b} are each independently selected from hydrogen and C₁-C₆ alkyl;
preferably, R^{a} and R^{b} are each independently selected from hydrogen and methyl;
preferably, Y is selected from —O—, —S—, —CH₂—, 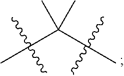 [SMILES: *C(*)(C)C]
more preferably, Y is selected from —O—, —S—;
n1 and n2 are each independently selected from 0 and 1, and n1 and n2 are not 0 at the same time;
L¹ and L² are each independently selected from the group consisting of a direct bond and C₆-C₁₈ arylene, and the C₆-C₁₈ arylene is optionally substituted by one R^{L};
R^{L} is selected from benzoxazolyl, benzothiazolyl, benzimidazolyl, benzisoxazolyl, benzisothiazolyl, and indazolyl;
R¹ and R² are each independently selected from the group consisting of diazaphenanthrolinyl, 6-membered nitrogen-containing heteroaryl, benzoxazolyl, benzothiazolyl, benzimidazolyl, benzisoxazolyl, benzisothiazolyl, indazolyl, and the diazaphenanthrolinyl, 6-membered nitrogen-containing heteroaryl, benzoxazolyl, benzothiazolyl, benzimidazolyl, benzisoxazolyl, benzisothiazolyl, indazolyl are each independently optionally substituted by one or two R^{R};
R^{R} is selected from C₆-C₁₈ aryl and fluorenyl, and the C₆-C₁₈ aryl and fluorenyl are each independently optionally substituted by 1 to 3 (preferably 1 to 2, more preferably 2) C₁-C₆ alkyl;
R³ and R⁴ are each independently selected from hydrogen and C₁-C₆ alkyl;
preferably, R³ and R⁴ are hydrogen.

2. (canceled)

3. (canceled)

4. (canceled)

5. The compound, or a stereoisomer or tautomer thereof according to claim 1, wherein L¹ and L² are each independently selected from the group consisting of a direct bond, phenylene, naphthylene, and biphenylene, and the phenylene, naphthylene, and biphenylene are each independently optionally substituted by one R^{L};
preferably, L¹, L² are each independently selected from the group consisting of a direct bond, phenylene, naphthylene, biphenylene, and the phenylene is optionally substituted by one R^{L};
more preferably, L¹ and L² are each independently selected from the group consisting of a direct bond, 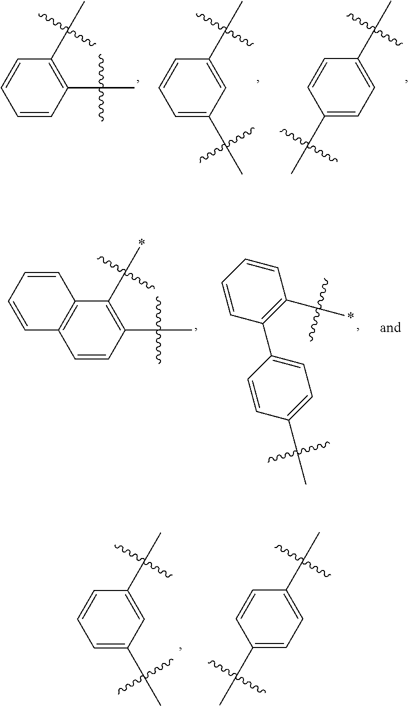
are each independently optionally substituted by one R^{L}, wherein the bond with marker * is bonded to the parent ring, and the bond without marker * is bonded to R¹ or R²;
R^{L} is selected from benzoxazolyl, benzothiazolyl;
preferably, R^{L} is benzoxazolyl;
more preferably, R^{L} is selected from 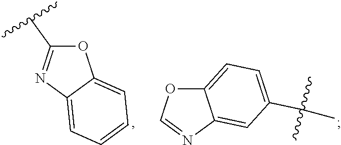
most preferably, L¹ and L² are each independently selected from the group consisting of:
a direct bond, 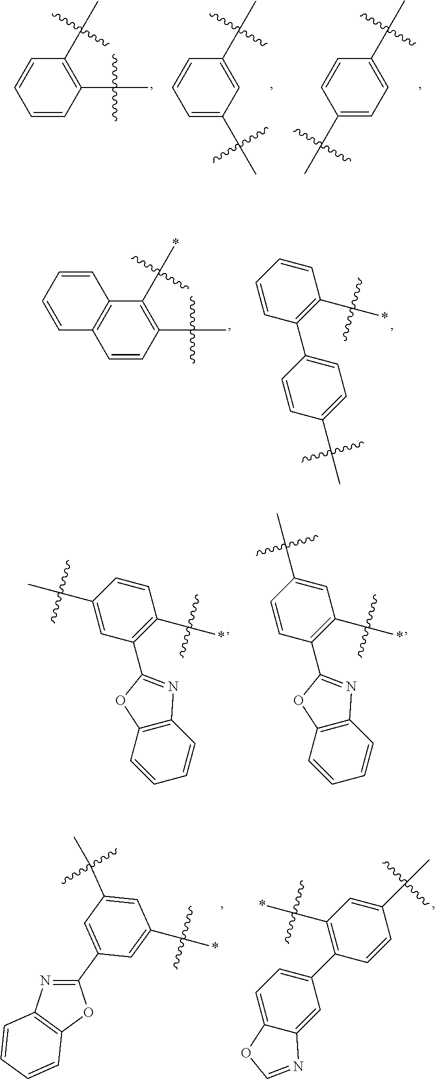
wherein, the bond with marker * is bonded to the parent ring, and the bond without marker * is bonded to R¹ or R².

6. The compound, or a stereoisomer or tautomer thereof according to claim 1, wherein R¹ and R² are each independently selected from the group consisting of diazaphenanthrolinyl, pyridinyl, pyrimidinyl, triazinyl, benzoxazolyl, benzothiazolyl, and the diazaphenanthrolinyl, pyridinyl, pyrimidinyl, triazinyl, benzoxazolyl and benzothiazolyl are each independently optionally substituted by one or two R^{R};
preferably, R¹ and R² are each independently selected from the group consisting of diazaphenanthrolinyl, pyridinyl, pyrimidinyl, triazinyl, benzoxazolyl, benzothiazolyl, and the pyridinyl, pyrimidinyl, triazinyl and benzoxazolyl are each independently optionally substituted by one or two R^{R};
more preferably, R¹ and R² are each independently selected from the group consisting of o-diazaphenanthrolinyl, pyridinyl, pyrimidinyl, 1,3,5-triazinyl, benzoxazolyl, and the pyridinyl, pyrimidinyl, 1,3,5-triazinyl, benzoxazolyl are each independently optionally substituted by one or two R^{R};
further preferably, R¹ and R² are each independently selected from the group consisting of 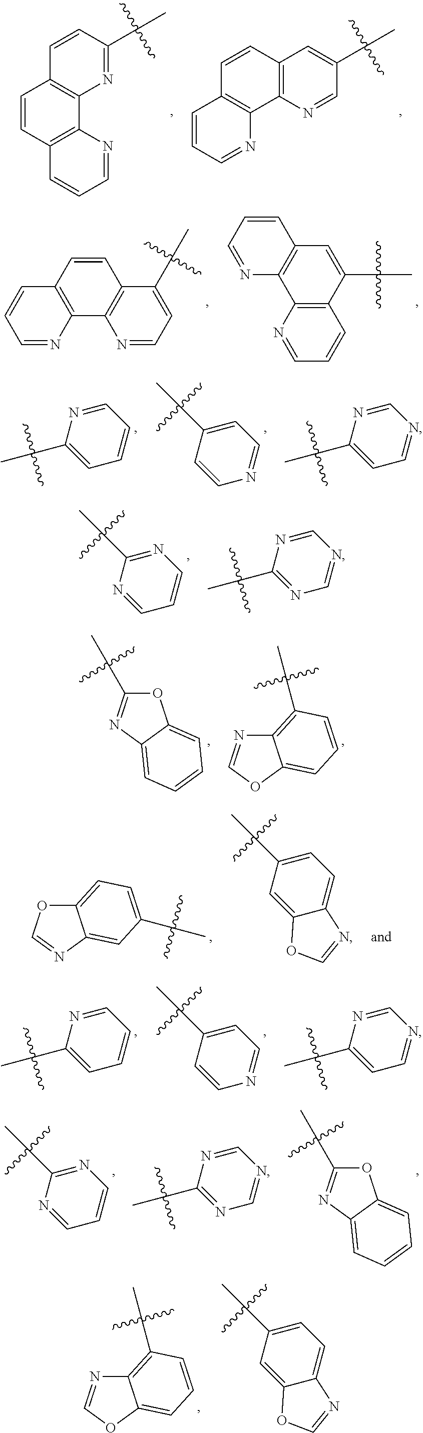
are each independently optionally substituted by one or two R^{R};
R^{R} is selected from the group consisting of phenyl, biphenyl, naphthyl, and fluorenyl, and the phenyl, biphenyl, naphthyl, and fluorenyl each are independently optionally substituted by two C₁-C₆ alkyl groups;
preferably, R^{R} is selected from the group consisting of phenyl, biphenyl, naphthyl, fluorenyl, and the fluorenyl is optionally substituted by two C₁-C₆ alkyl groups;
more preferably, R^{R} is selected from the group consisting of phenyl, biphenyl, naphthyl, fluorenyl, and the fluorenyl is optionally substituted by two methyl groups;
most preferably, R^{R} is selected from the group consisting of 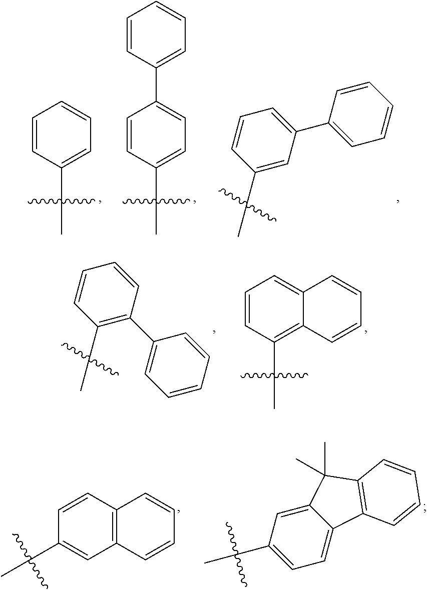
most preferably, R¹ and R² are each independently selected from the group consisting

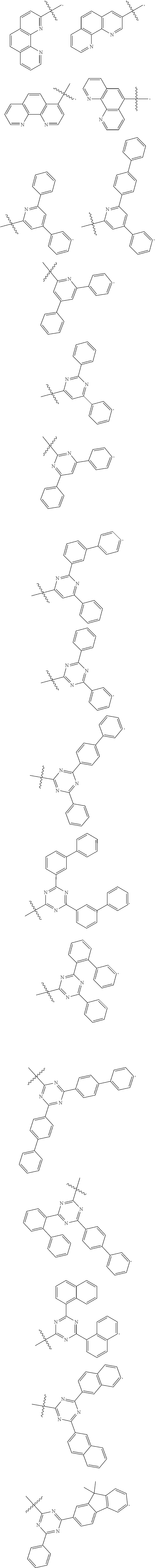

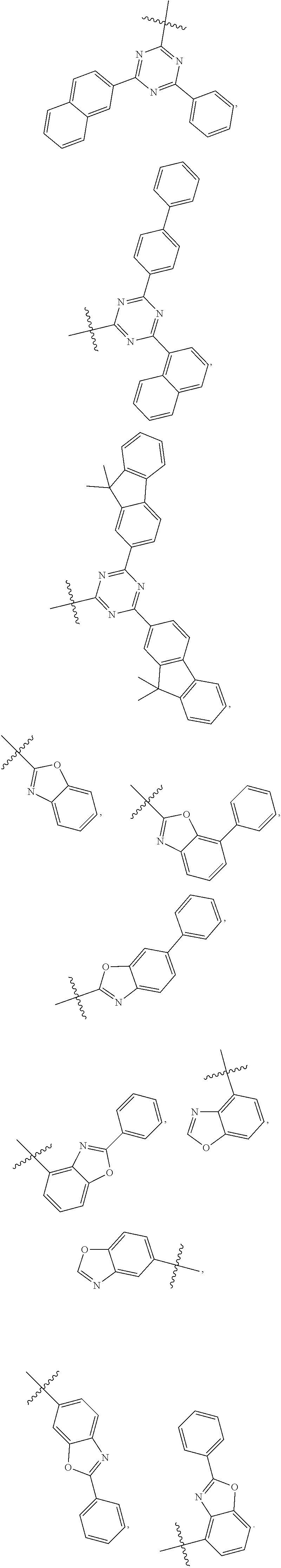

7. The compound, or a stereoisomer or tautomer thereof according to claim 1, wherein R¹-L¹- and R²-L²- are each independently selected from the group consisting of:

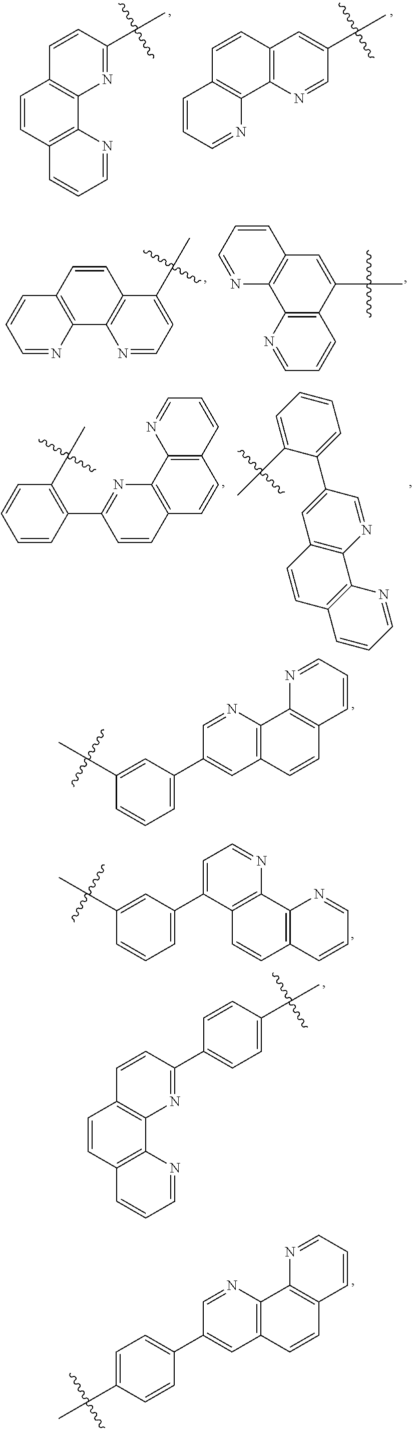

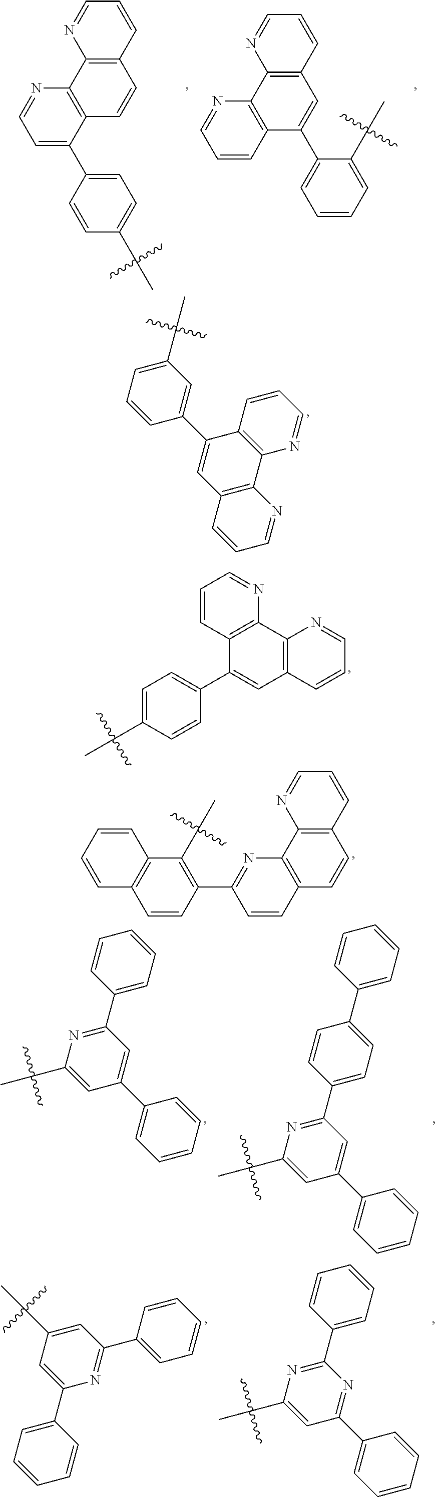

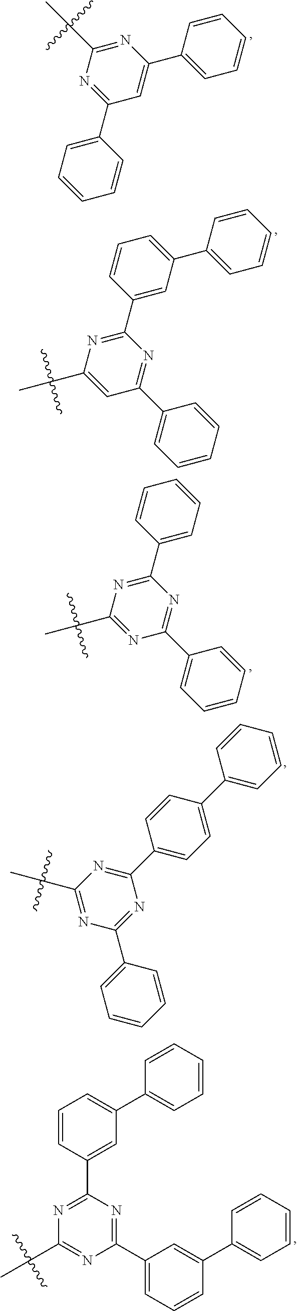

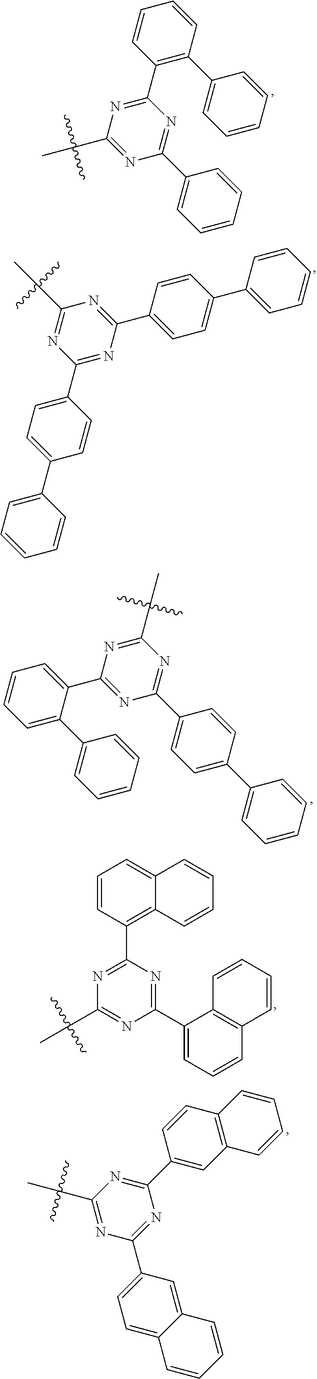

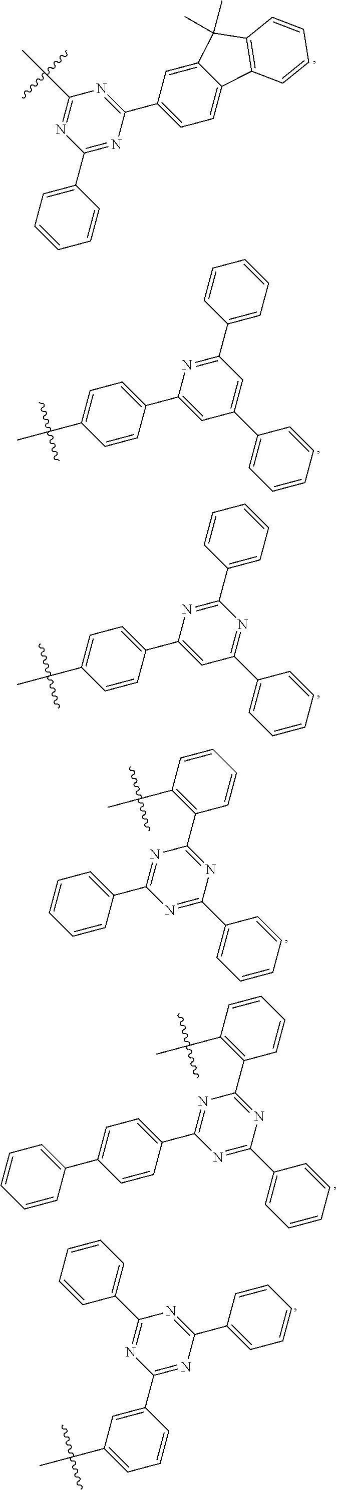

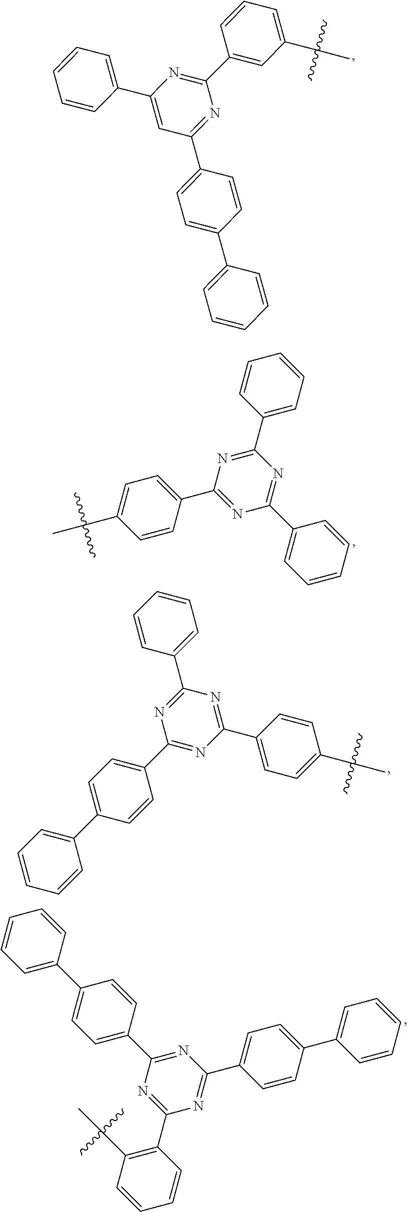

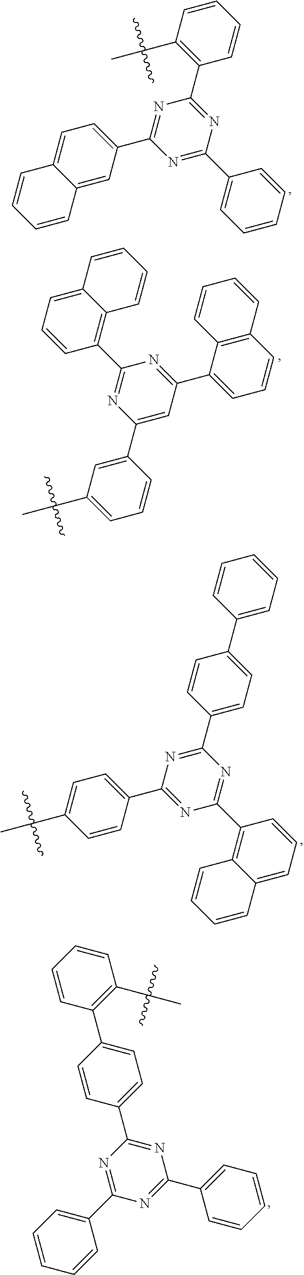

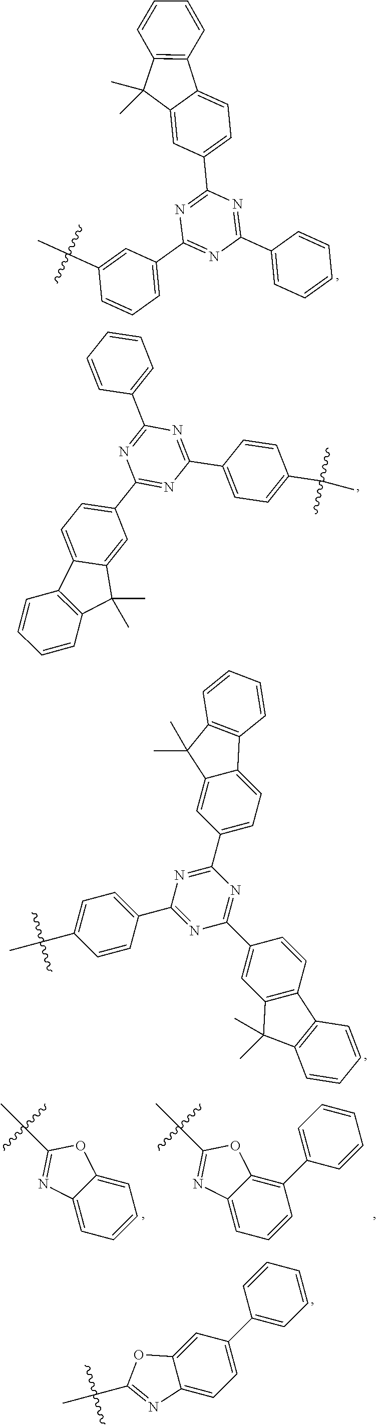

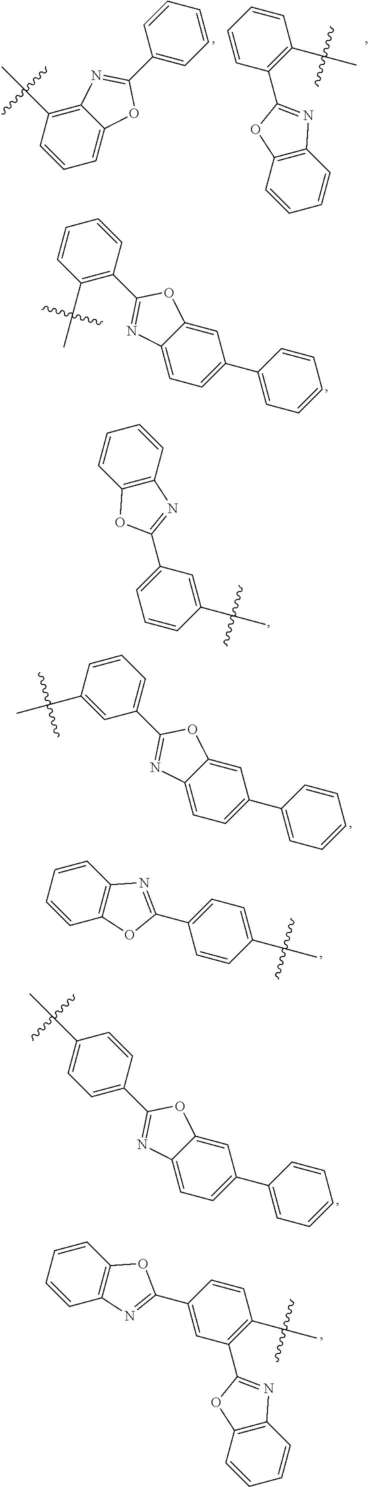

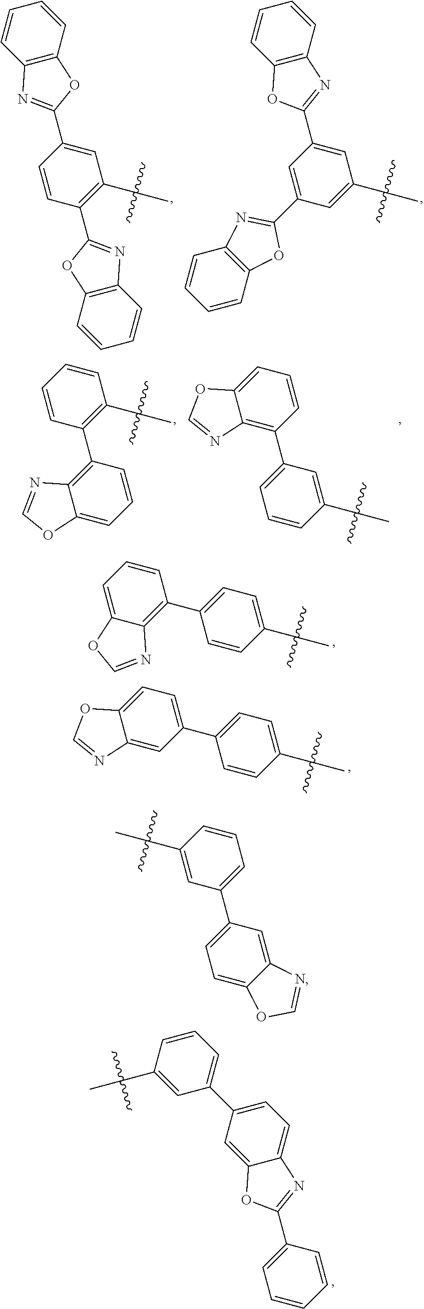

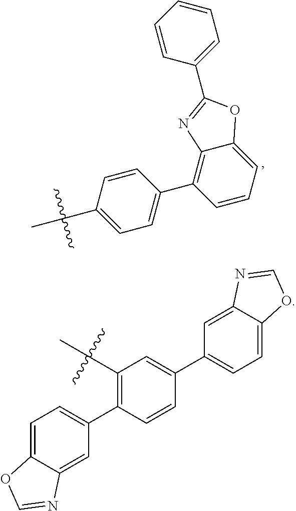 [SMILES: *c1ccc(-c2cccc3oc(-c4ccccc4)nc23)cc1 ; *c1cc(-c2ccc3ocnc3c2)ccc1-c1ccc2ocnc2c1]

8. The compound, or a stereoisomer or tautomer thereof according to claim 1, wherein the compound has the structure represented by Formula I, 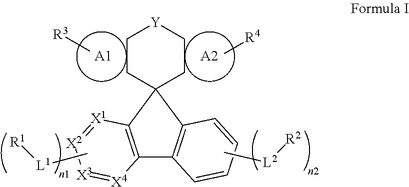
wherein:
X¹, X², X³, and X⁴ are each independently selected from CH and N, and at least one of X¹, X², X³, and X⁴ is N, and X³ and X⁴ are not N at the same time;
preferably, X¹, X², X³ and X⁴ are each independently selected from CH and N, and two of X¹, X², X³ and X⁴ are N, and X³ and X⁴ are not N at the same time;
n1 and n2 are each independently selected from 0 and 1, and n1 and n2 are not 0 at the same time;
preferably, 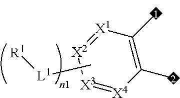
is selected from 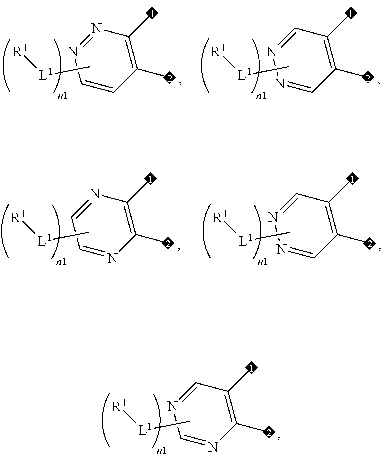
wherein Carbon atom No. 1 is bonded to the central spiro carbon atom, and Carbon atom No. 2 is bonded to a carbon atom of the aromatic ring on the right;
more preferably, 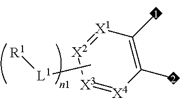
is selected from the group consisting of: 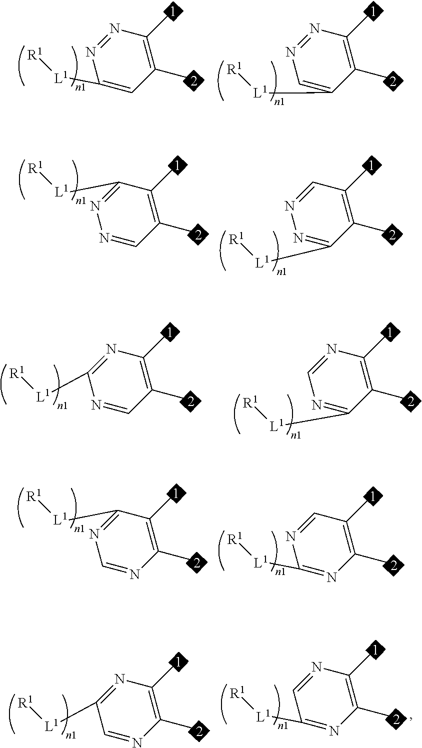
wherein, Carbon atom No. 1 is bonded to the central spiro carbon atom, and Carbon atom No. 2 is bonded to a carbon atom of the aromatic ring on the right;
preferably, 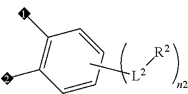
is selected from 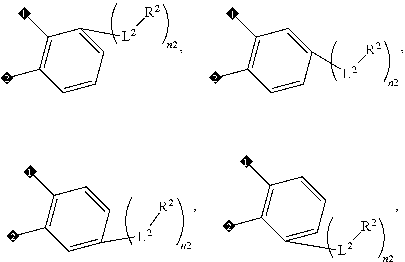
wherein Carbon atom No. 1 is bonded to the central spiro carbon atom, and Carbon atom No. 2 is bonded to a carbon atom of the aromatic ring on the left;
Ring A1 is selected from benzene ring and naphthalene ring;
preferably, Ring A1 is selected from the group consisting of 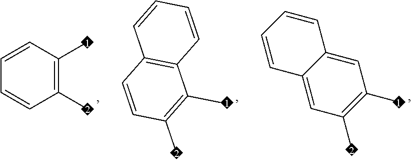
wherein Carbon atom No. 1 is fused with the carbon atom adjacent to Y on the middle 6-membered ring, and Carbon atom No. 2 is fused with the carbon atom far away from Y on the middle 6-membered ring;
more preferably, Ring A1 is 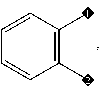
wherein Carbon atom No. 1 is fused with the carbon atom adjacent to Y on the middle 6-membered ring, and Carbon atom No. 2 is fused with the carbon atom far away from Y on the middle 6-membered ring;
Ring A2 is selected from benzene ring and naphthalene ring;
preferably, Ring A2 is selected from the group consisting of 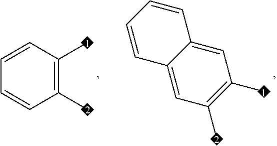
wherein Carbon atom No. 1 is fused with the carbon atom adjacent to Y on the middle 6-membered ring, and Carbon atom No. 2 is fused with the carbon atom far away from Y on the middle 6-membered ring;
more preferably, Ring A2 is 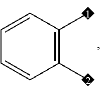
wherein Carbon atom No. 1 is fused with the carbon atom adjacent to Y on the middle 6-membered ring, and Carbon atom No. 2 is fused with the carbon atom far away from Y on the middle 6-membered ring;
Y is selected from —O—, —S—, —CR^{a}R^{b}—;
R^{a} and R^{b} are each independently selected from hydrogen and C₁-C₆ alkyl;
preferably, R^{a} and R^{b} are each independently selected from hydrogen and methyl;
preferably, Y is selected from —O—, —S—, —CH₂—, 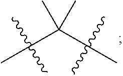 [SMILES: *C(*)(C)C]
more preferably, Y is selected from —O—, —S—;
L¹ and L² are each independently selected from the group consisting of a direct bond, phenylene, naphthylene, biphenylene, and the phenylene, naphthylene, and biphenylene are each independently optionally substituted by one R^{L};
preferably, L¹ and L² are each independently selected from the group consisting of a direct bond, phenylene, naphthylene, and biphenylene, and the phenylene is optionally substituted by one R^{L};
more preferably, L¹ and L² are each independently selected from the group consisting of a direct bond, 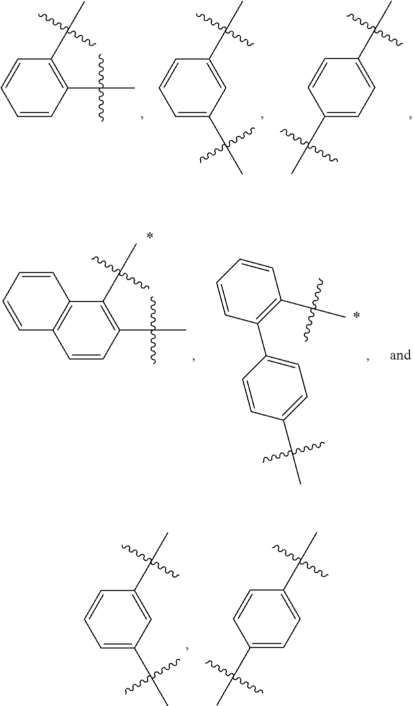
are each independently optionally substituted by one R^{L}, wherein the bond with marker * is bonded to the parent ring, and the bond without marker * is bonded to R¹ or R²;
R^{L} is selected from benzoxazolyl and benzothiazolyl;
preferably, R^{L} is benzoxazolyl;
more preferably, R^{L} is 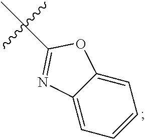 [SMILES: *c1nc2ccccc2o1]
most preferably, L¹ and L² are each independently selected from the group consisting of a direct bond, 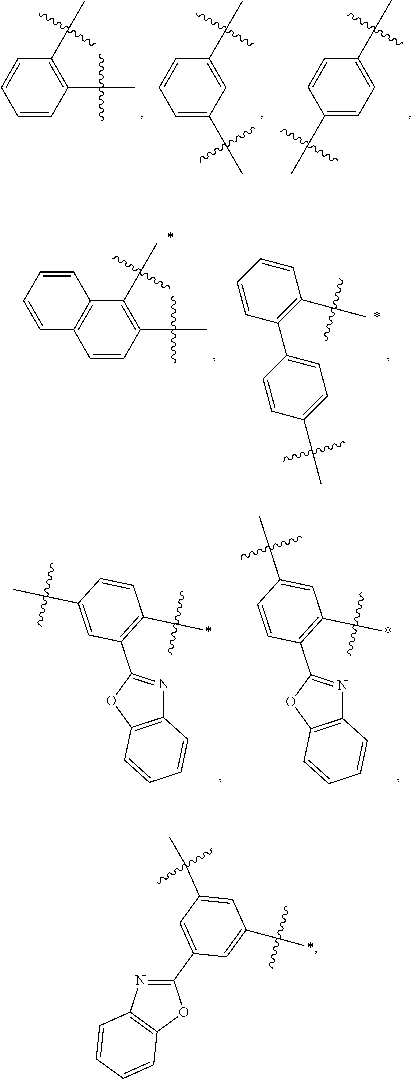
wherein the bond with marker * is bonded to the parent ring, and the bond without marker * is bonded to R¹ or R²;
R¹ and R² are each independently selected from the group consisting of diazaphenanthrolinyl, pyridinyl, pyrimidinyl, triazinyl, benzoxazolyl, benzothiazolyl, and the diazaphenanthrolinyl, pyridinyl, pyrimidinyl, triazinyl, benzoxazolyl and benzothiazolyl are each independently optionally substituted by one or two (preferably two) R^{R};
preferably, R¹ and R² are each independently selected from the group consisting of diazaphenanthrolinyl, pyridinyl, pyrimidinyl, triazinyl, benzoxazolyl, benzothiazolyl, and the pyridinyl, pyrimidinyl, triazinyl are each independently optionally substituted by one or two (preferably two) R^{R};
more preferably, R¹ and R² are each independently selected from the group consisting of o-diazaphenanthrolinyl, pyridinyl, pyrimidinyl, 1,3,5-triazinyl, and benzoxazolyl, and the pyridinyl, pyrimidinyl, and 1,3,5-triazinyl are each independently optionally substituted by one or two (preferably two) R^{R};
further preferably, R¹ and R² are each independently selected from the group consisting of 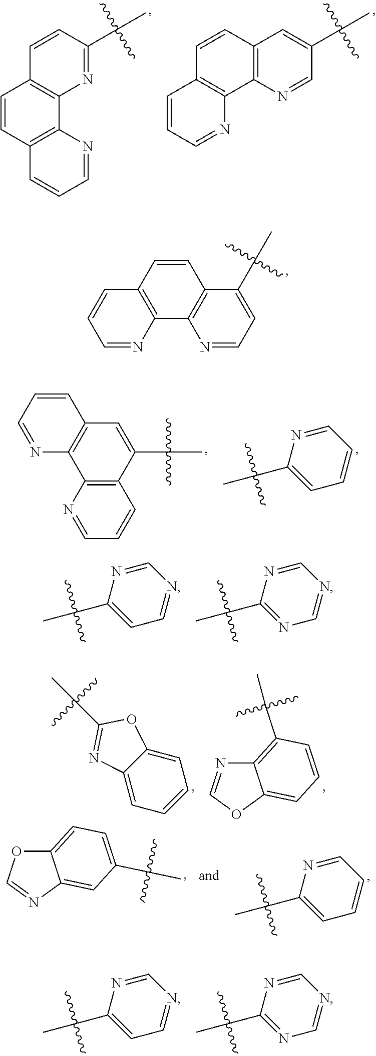
are each independently optionally substituted by one or two (preferably two) R^{R};
R^{R} is selected from the group consisting of phenyl, biphenyl, naphthyl, and fluorenyl, and the phenyl, biphenyl, naphthyl, and fluorenyl are each independently optionally substituted by two C₁-C₆ alkyl groups;
preferably, R^{R} is selected from the group consisting of phenyl, biphenyl, naphthyl, and fluorenyl, and the fluorenyl is optionally substituted by two C₁-C₆ alkyl groups;
more preferably, R^{R} is selected from the group consisting of phenyl, biphenyl, naphthyl, fluorenyl, and the fluorenyl is optionally substituted by two methyl groups;
most preferably, R^{R} is selected from the group consisting of 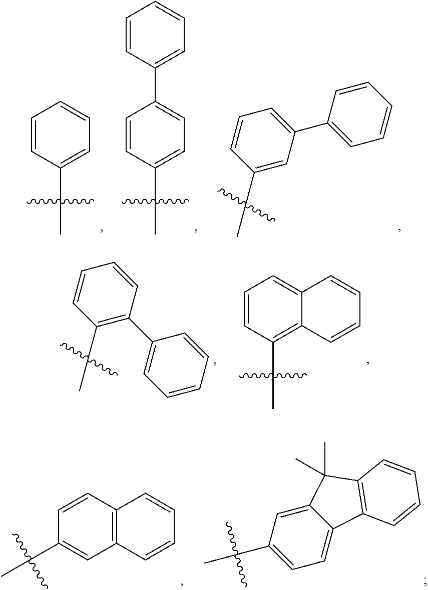
most preferably, R¹ and R² are each independently selected from the group consisting 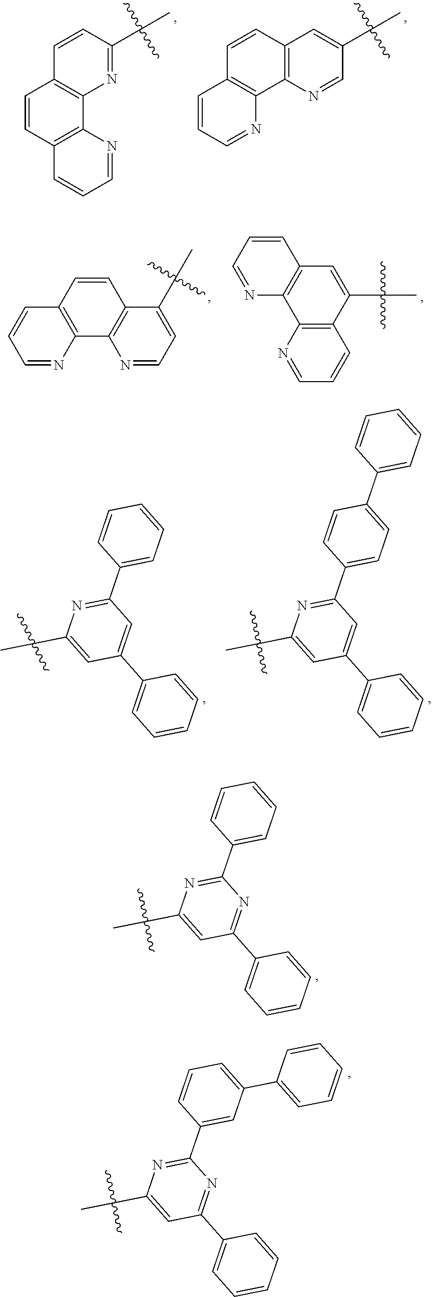 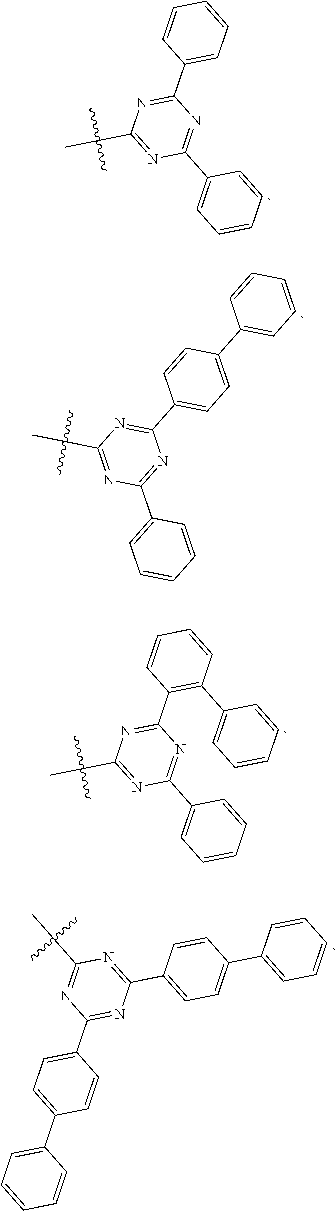 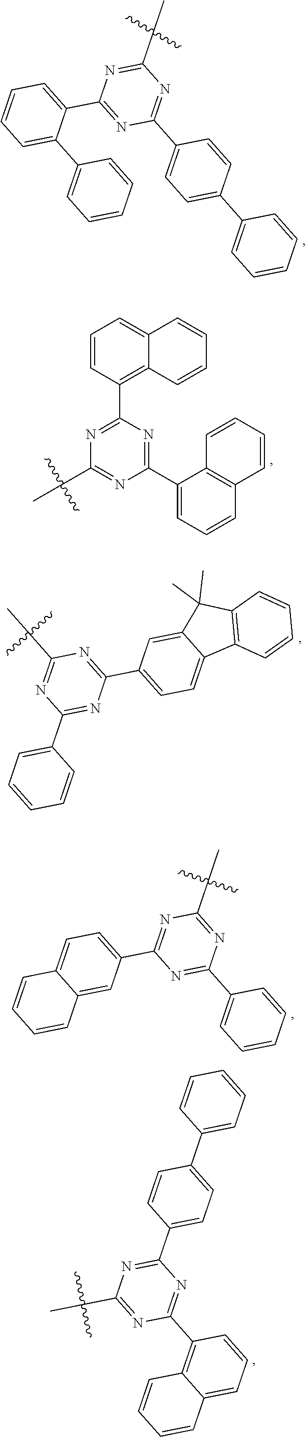 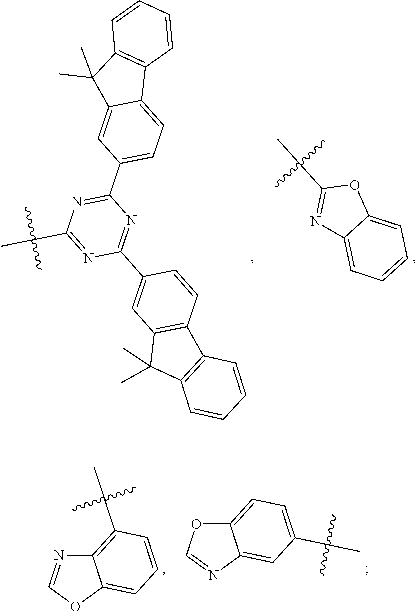
most preferably, R¹-L¹-, R²-L²- are each independently selected from the group consisting of: 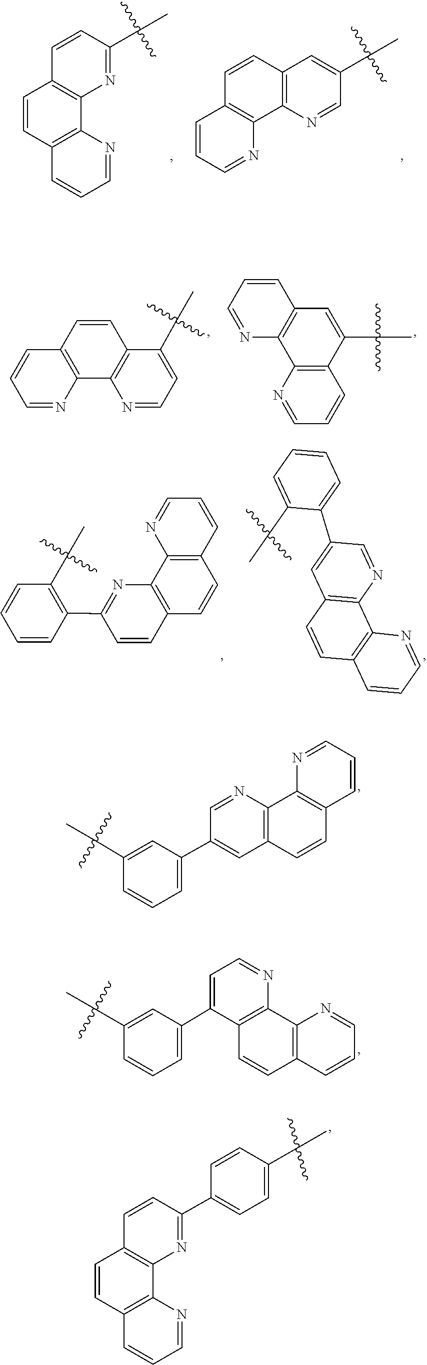 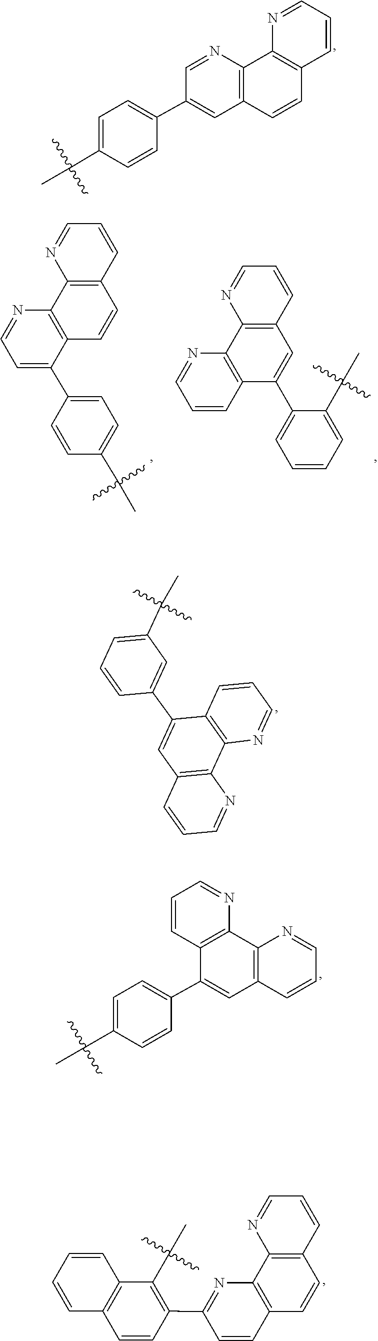 [SMILES: *c1ccc(-c2cnc3c(ccc4cccnc43)c2)cc1 ; *c1ccc(-c2ccnc3c2ccc2cccnc23)cc1 ; *c1ccccc1-c1cc2cccnc2c2ncccc12 ; *c1cccc(-c2cc3cccnc3c3ncccc23)c1 ; *c1ccc(-c2cc3cccnc3c3ncccc23)cc1 ; *c1c(-c2ccc3ccc4cccnc4c3n2)ccc2ccccc12] 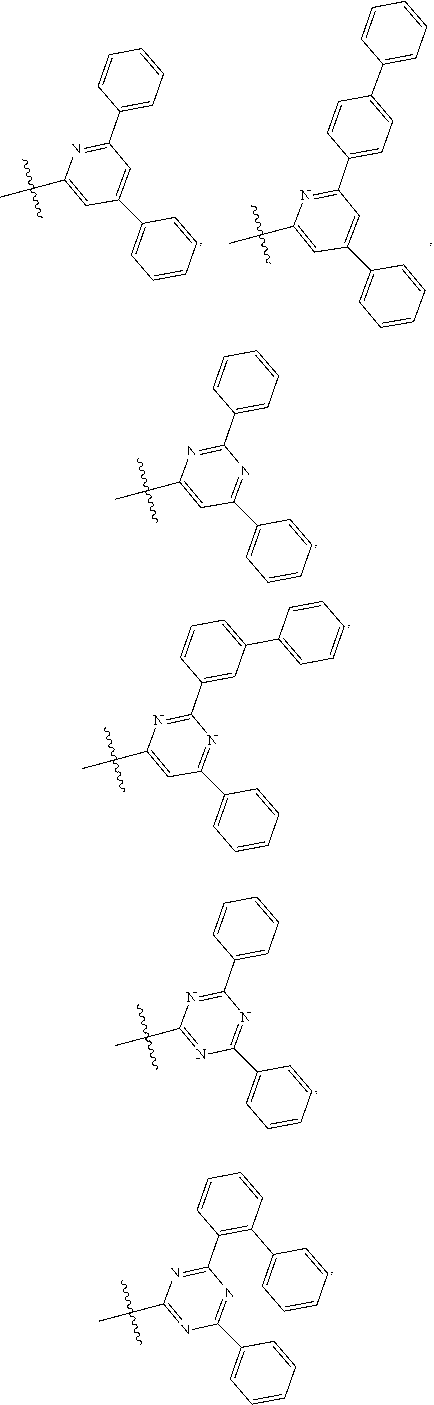 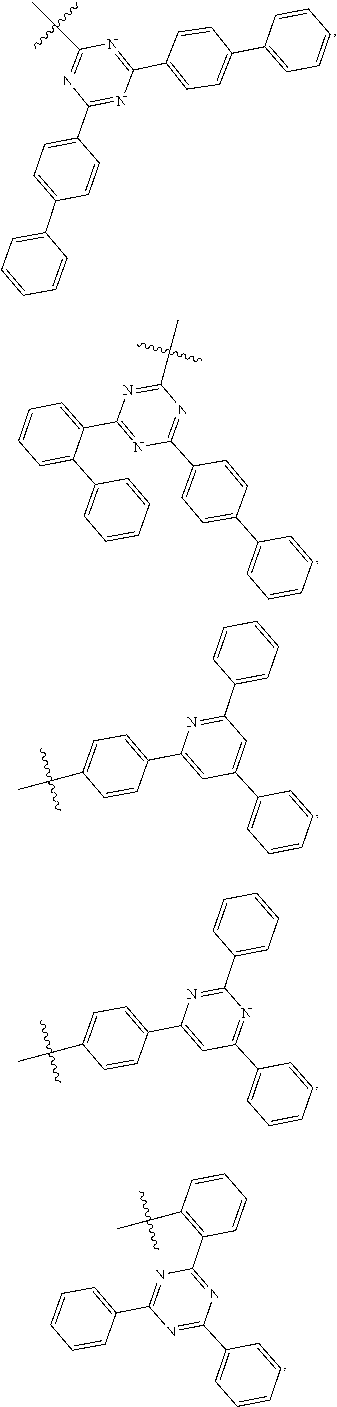 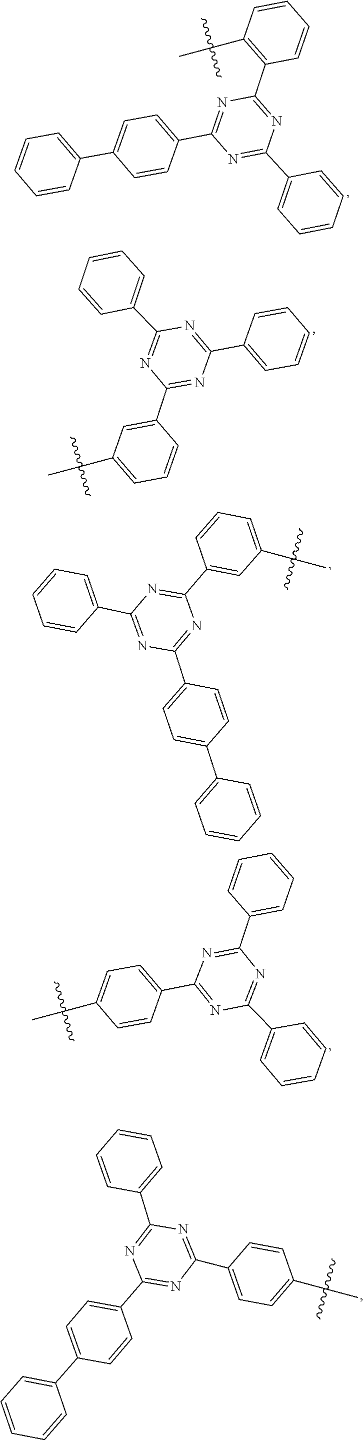 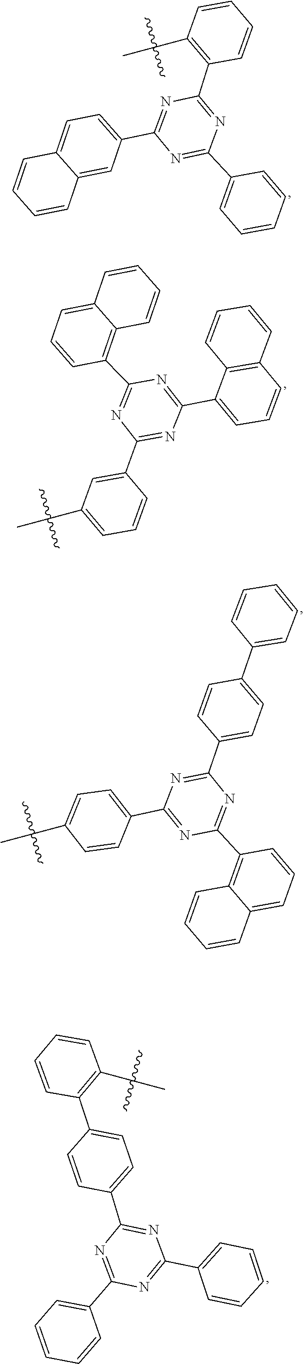 [SMILES: *c1ccccc1-c1nc(-c2ccccc2)nc(-c2ccc3ccccc3c2)n1 ; *c1cccc(-c2nc(-c3cccc4ccccc34)nc(-c3cccc4ccccc34)n2)c1 ; *c1ccc(-c2nc(-c3ccc(-c4ccccc4)cc3)nc(-c3cccc4ccccc34)n2)cc1 ; *c1ccccc1-c1ccc(-c2nc(-c3ccccc3)nc(-c3ccccc3)n2)cc1] 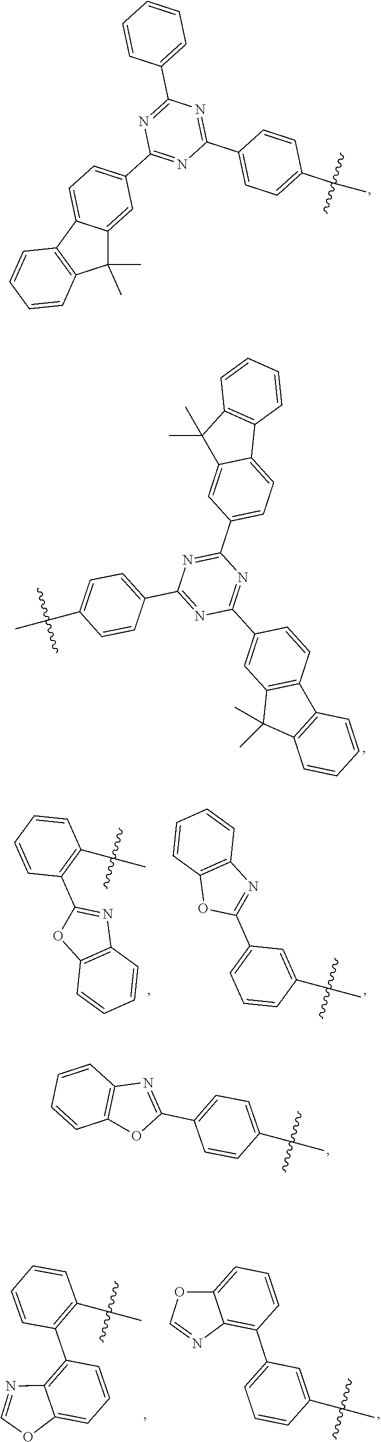 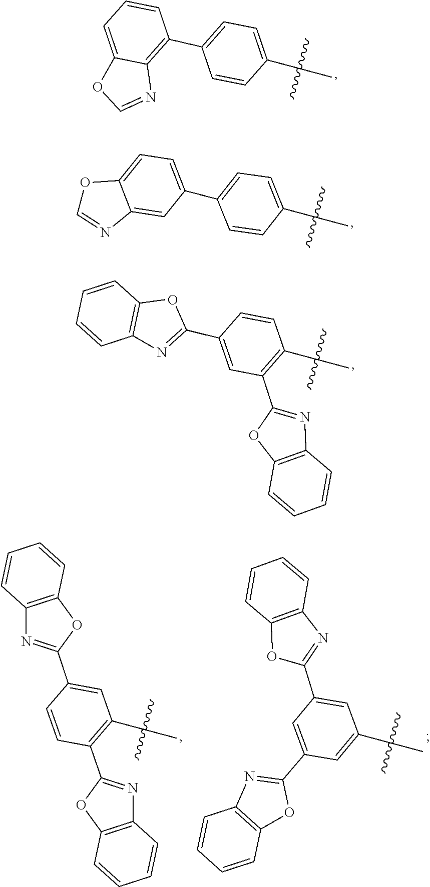
R³ and R⁴ are each independently selected from hydrogen and C₁-C₆ alkyl;
preferably, R³ and R⁴ are hydrogen.

9. The compound, or a stereoisomer or tautomer thereof according to claim 8, wherein the compound has the structure represented by Formula I-1, 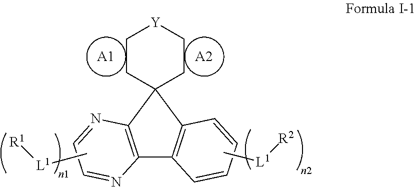
wherein:
Ring A1 is selected from benzene ring and naphthalene ring;
preferably, Ring A1 is selected from the group consisting of 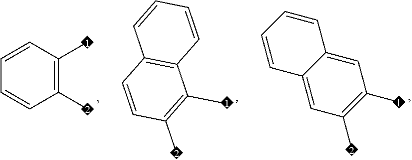
wherein Carbon atom No. 1 is fused with the carbon atom adjacent to Y on the middle 6-membered ring, and Carbon atom No. 2 is fused with the carbon atom far away from Y on the middle 6-membered ring;
more preferably, Ring A1 is 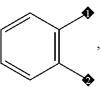
wherein Carbon atom No. 1 is fused with the carbon atom adjacent to Y on the middle 6-membered ring, and Carbon atom No. 2 is fused with the carbon atom far away from Y on the middle 6-membered ring;
Ring A2 is selected from benzene ring and naphthalene ring;
preferably, Ring A2 is selected from the group consisting of 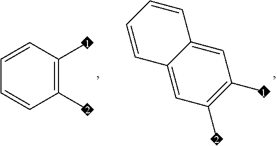
wherein Carbon atom No. 1 is fused with the carbon atom adjacent to Y on the middle 6-membered ring, and Carbon atom No. 2 is fused with the carbon atom far away from Y on the middle 6-membered ring;
more preferably, Ring A2 is 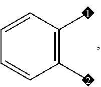
wherein Carbon atom No. 1 is fused with the carbon atom adjacent to Y on the middle 6-membered ring, and Carbon atom No. 2 is fused with the carbon atom far away from Y on the middle 6-membered ring;
Y is selected from —O—, —S—, —CR^{a}R^{b}—;
R^{a} and R^{b} are each independently selected from hydrogen and C₁-C₆ alkyl;
preferably, R^{a} and R^{b} are each independently selected from hydrogen and methyl;
preferably, Y is selected from —O—, —S—, —CH₂—, 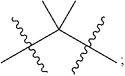 [SMILES: *C(*)(C)C]
more preferably, Y is selected from —O—, —S—;
n1 and n2 are each independently selected from 0 and 1, and n1 and n2 are not 0 at the same time;
preferably, 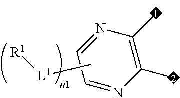
is selected from 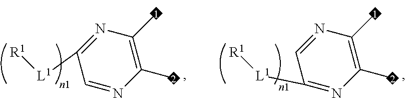
wherein Carbon atom No. 1 is bonded to the central spiro carbon atom, and Carbon atom No. 2 is bonded to a carbon atom of the aromatic ring on the right;
preferably, 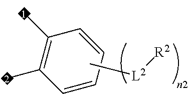
is selected from the group consisting of 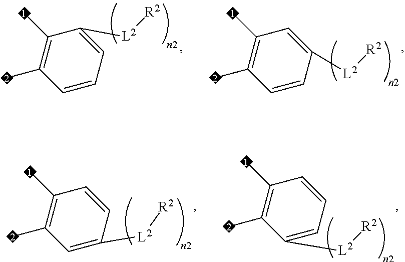
wherein Carbon atom No. 1 is bonded to the central spiro carbon atom, and Carbon atom No. 2 is bonded to a carbon atom of the aromatic ring on the left;
L¹ and L² are each independently selected from the group consisting of a direct bond, phenylene, naphthylene, and biphenylene, and the phenylene, naphthylene, and biphenylene are each independently optionally substituted by one R^{L};
preferably, L¹ and L² are each independently selected from the group consisting of a direct bond, phenylene, naphthylene, and biphenylene, and the phenylene is optionally substituted by one R^{L};
more preferably, L¹ and L² are each independently selected from the group consisting of a direct bond, 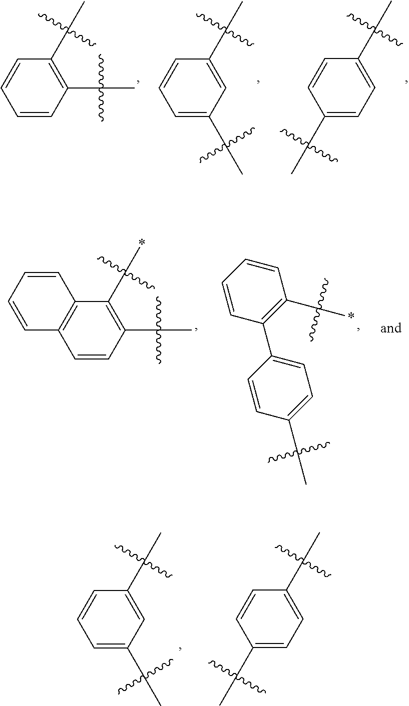
are each independently optionally substituted by one R^{L}, wherein the bond with marker * is bonded to the parent ring, and the bond without marker * is bonded to R¹ or R²;
R^{L} is selected from benzoxazolyl and benzothiazolyl;
preferably, R^{L} is benzoxazolyl;
more preferably, R^{L} is 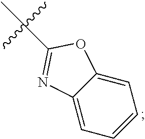 [SMILES: *c1nc2ccccc2o1]
most preferably, L¹ and L² are each independently selected from the group consisting of a direct bond, 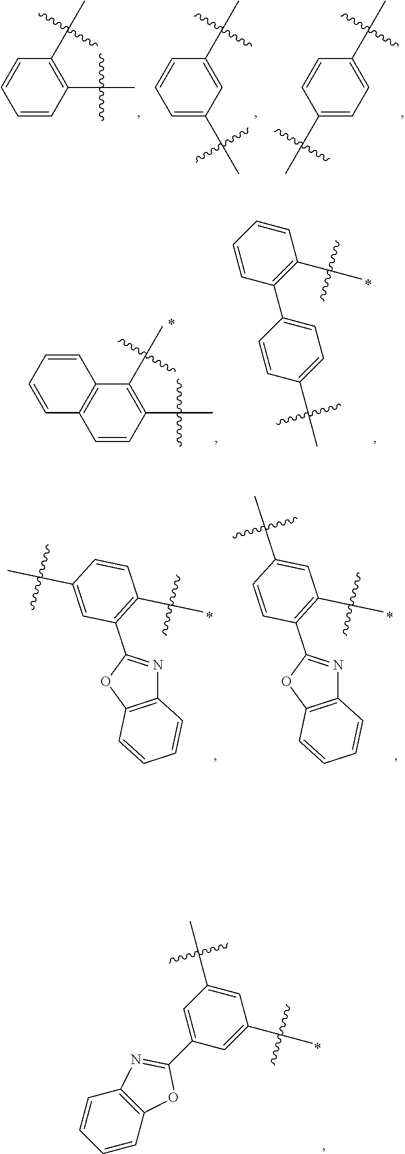
wherein the bond with marker * is bonded to the parent ring, and the bond without marker * is bonded to R¹ or R²;
R¹ and R² are each independently selected from the group consisting of diazaphenanthrolinyl, pyridinyl, pyrimidinyl, triazinyl, benzoxazolyl, benzothiazolyl, and the diazaphenanthrolinyl, pyridinyl, pyrimidinyl, triazinyl, benzoxazolyl and benzothiazolyl are each independently optionally substituted by one or two (preferably two) R^{R};
preferably, R¹ and R² are each independently selected from the group consisting of diazaphenanthrolinyl, pyridinyl, pyrimidinyl, triazinyl, benzoxazolyl, benzothiazolyl, and the pyridinyl, pyrimidinyl, triazinyl are each independently optionally substituted by one or two (preferably two) R^{R};
more preferably, R¹ and R² are each independently selected from the group consisting of o-diazaphenanthrolinyl, pyridinyl, pyrimidinyl, 1,3,5-triazinyl, and benzoxazolyl, and the pyridinyl, pyrimidinyl, and 1,3,5-triazinyl are each independently optionally substituted by one or two (preferably two) R^{R};
further preferably, R¹ and R² are each independently selected from the group consisting of 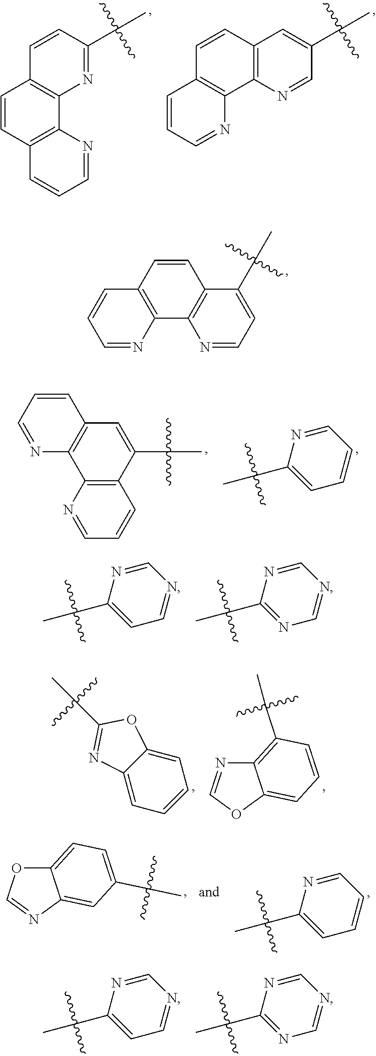
are each independently optionally substituted by one or two (preferably two) R^{R};
R^{R} is selected from the group consisting of phenyl, biphenyl, naphthyl, and fluorenyl, and the phenyl, biphenyl, naphthyl, and fluorenyl are each independently optionally substituted by two C₁-C₆ alkyl groups;
preferably, R^{R} is selected from the group consisting of phenyl, biphenyl, naphthyl, and fluorenyl, and the fluorenyl is optionally substituted by two C₁-C₆ alkyl groups;
more preferably, R^{R} is selected from the group consisting of phenyl, biphenyl, naphthyl, and fluorenyl, and the fluorenyl is optionally substituted by two methyl groups;
most preferably, R^{R} is selected from the group consisting of 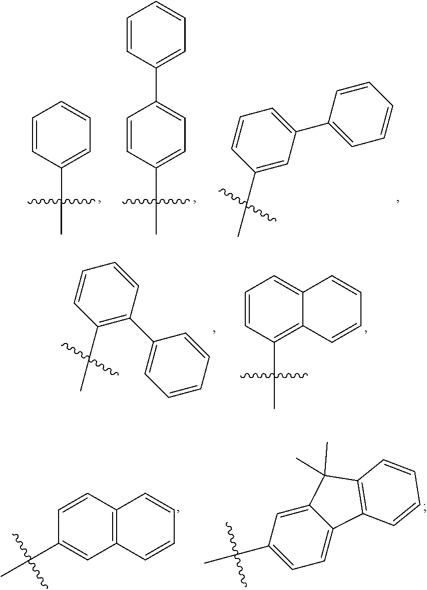
most preferably, R¹ and R² are each independently selected from the group consisting 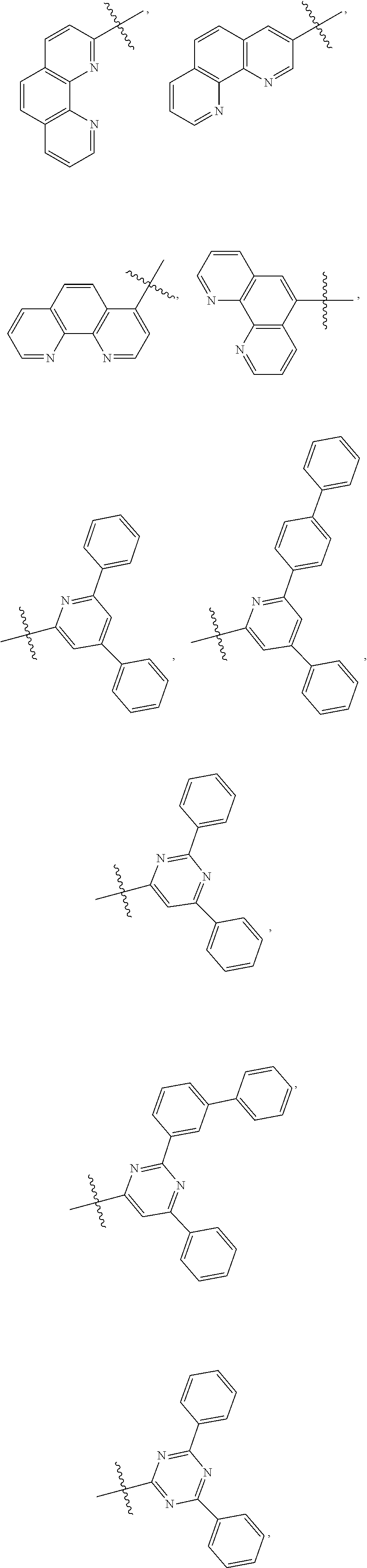 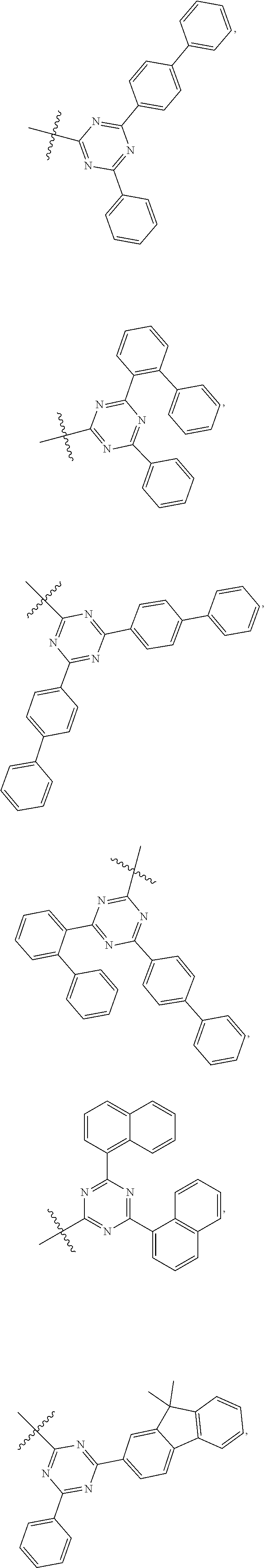 [SMILES: *c1nc(-c2ccccc2)nc(-c2ccc(-c3ccccc3)cc2)n1 ; *c1nc(-c2ccccc2)nc(-c2ccccc2-c2ccccc2)n1 ; *c1nc(-c2ccc(-c3ccccc3)cc2)nc(-c2ccc(-c3ccccc3)cc2)n1 ; *c1nc(-c2ccc(-c3ccccc3)cc2)nc(-c2ccccc2-c2ccccc2)n1 ; *c1nc(-c2cccc3ccccc23)nc(-c2cccc3ccccc23)n1 ; *c1nc(-c2ccccc2)nc(-c2ccc3c(c2)C(C)(C)c2ccccc2-3)n1] 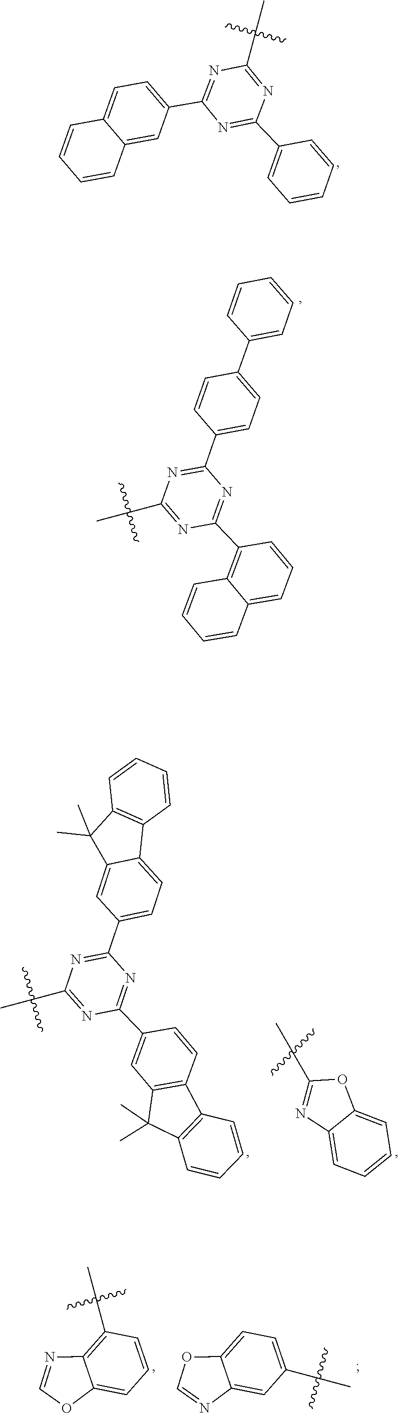
most preferably, R¹-L¹-, R²-L²- are each independently selected from the group

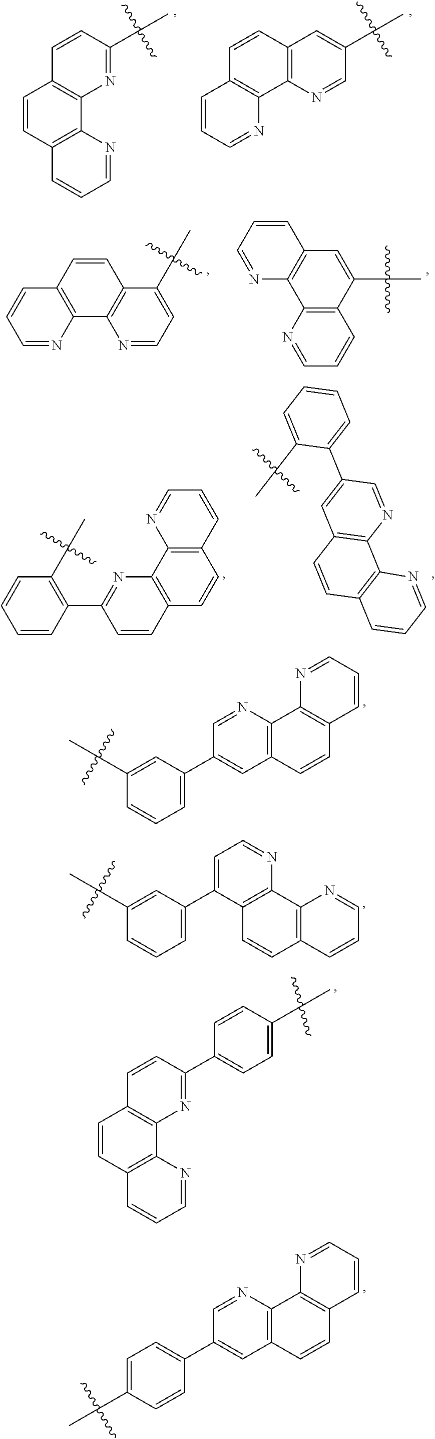

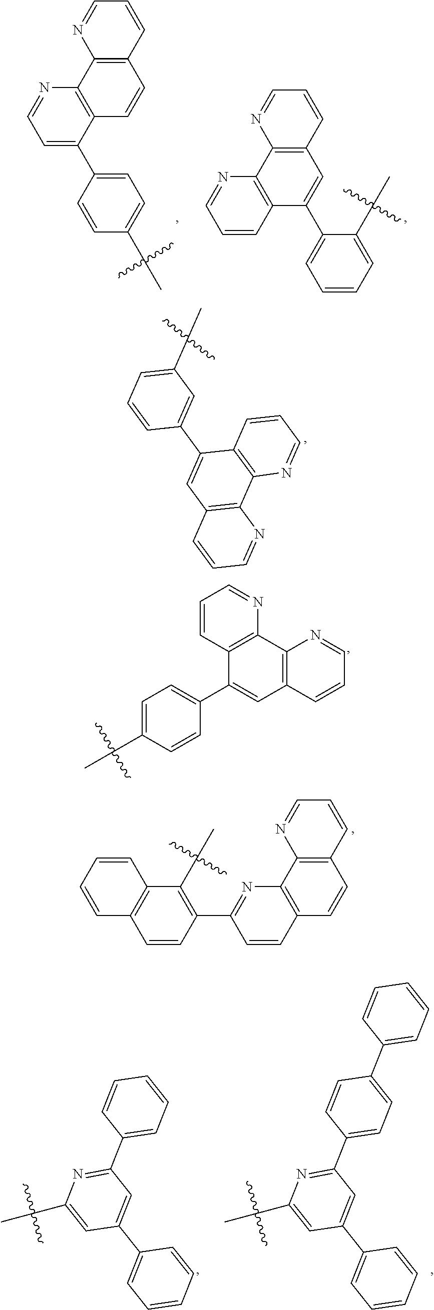

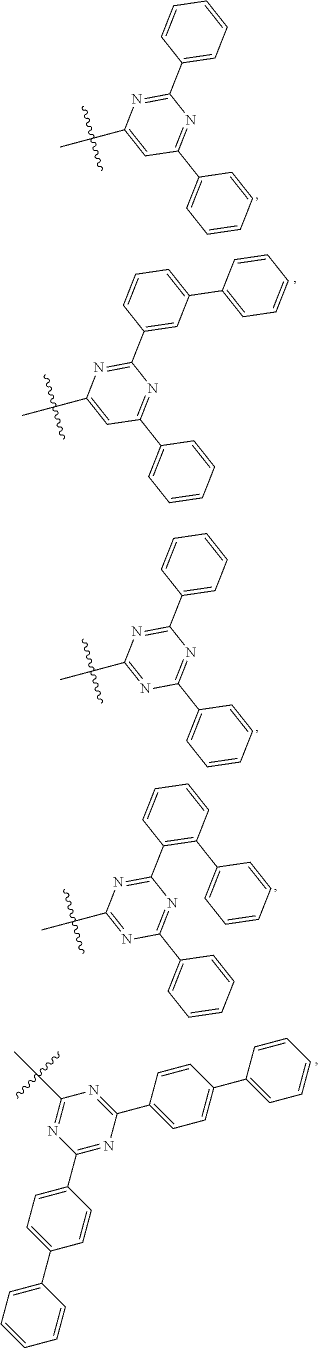

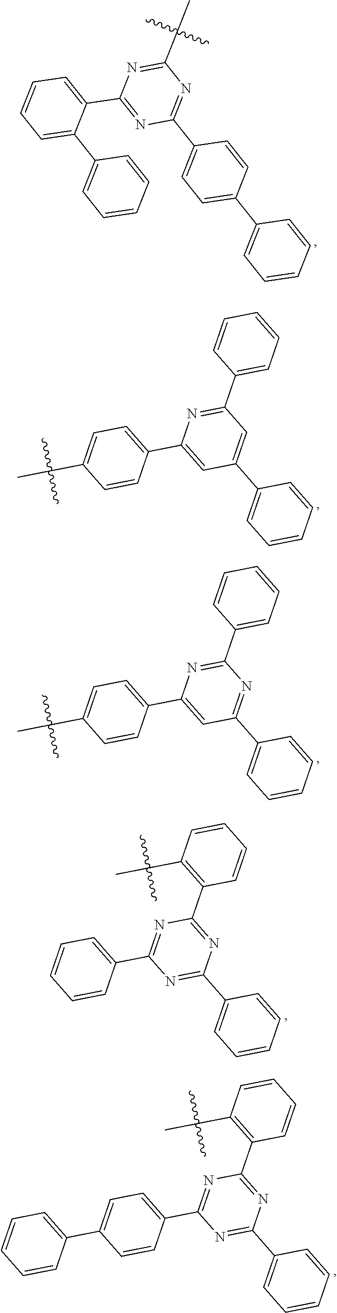

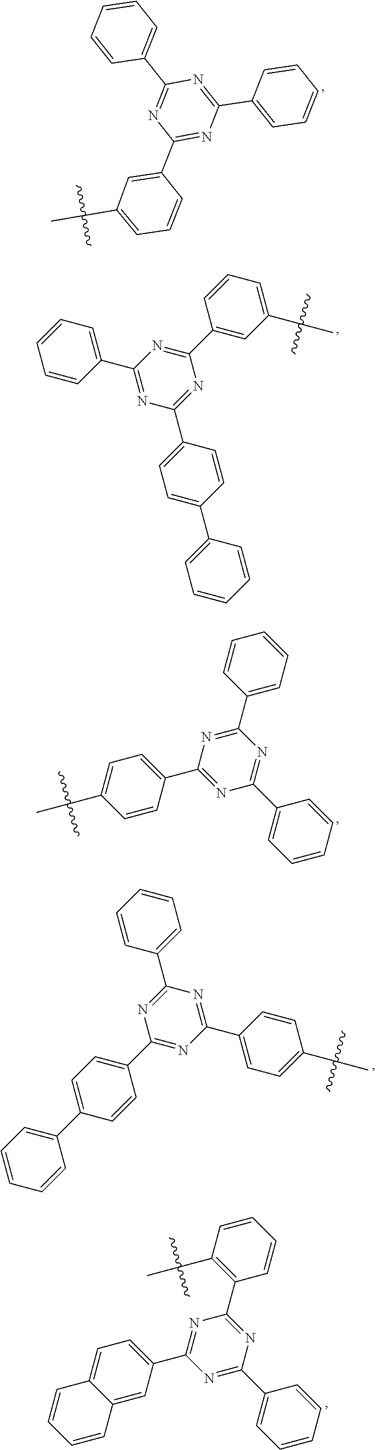

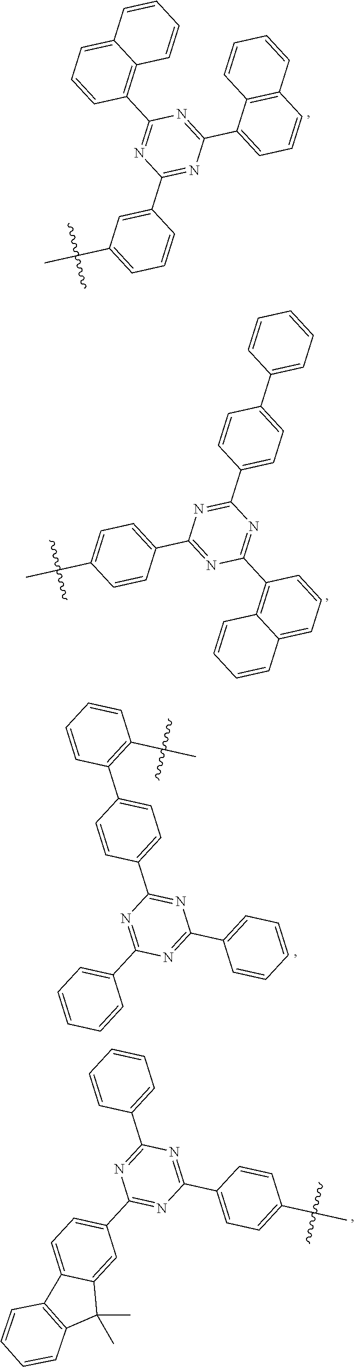

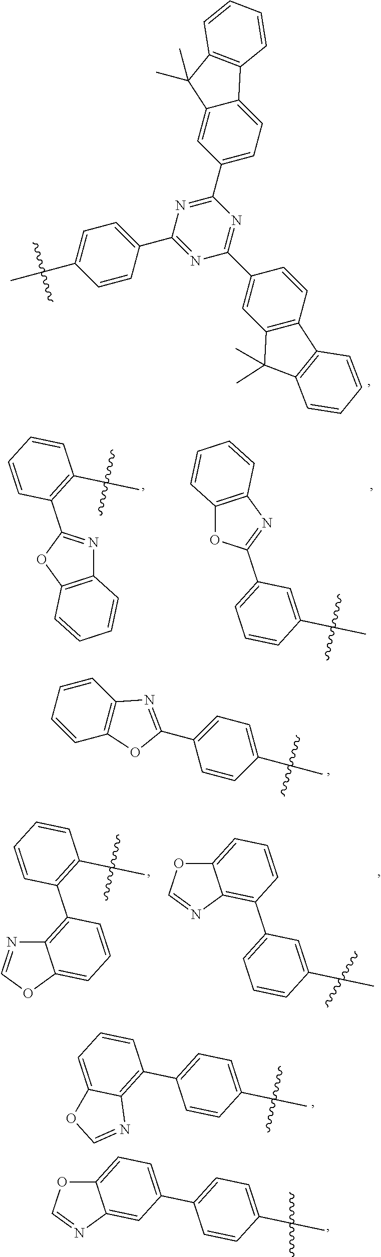

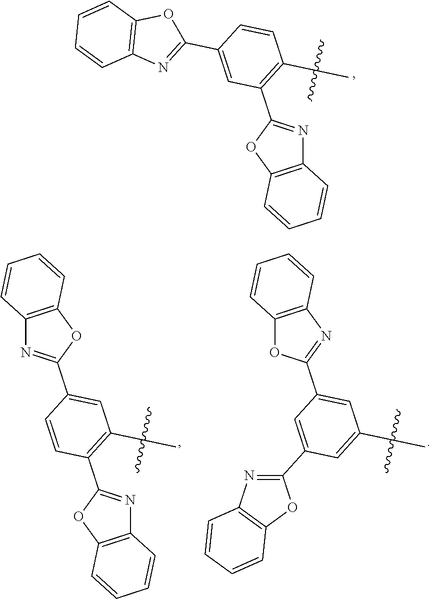

10. The compound, or a stereoisomer or tautomer thereof according to claim 8, wherein the compound has the structure represented by Formula I-2, 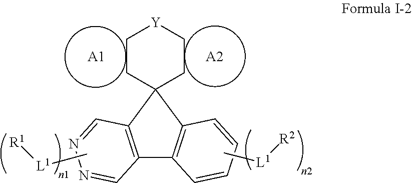
wherein:
Ring A1 is selected from benzene ring and naphthalene ring;
preferably, Ring A1 is selected from the group consisting of 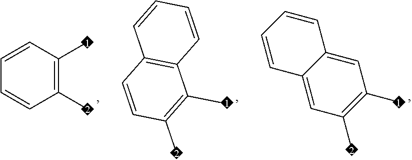
wherein Carbon atom No. 1 is fused with the carbon atom adjacent to Y on the middle 6-membered ring, and Carbon atom No. 2 is fused with the carbon atom far away from Y on the middle 6-membered ring;
more preferably, Ring A1 is 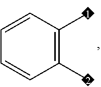
wherein Carbon atom No. 1 is fused with the carbon atom adjacent to Y on the middle 6-membered ring, and Carbon atom No. 2 is fused with the carbon atom far away from Y on the middle 6-membered ring;
Ring A2 is selected from benzene ring and naphthalene ring;
preferably, Ring A2 is selected from the group consisting of 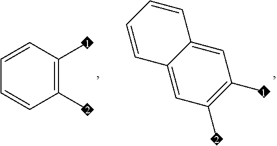
wherein Carbon atom No. 1 is fused with the carbon atom adjacent to Y on the middle 6-membered ring, and Carbon atom No. 2 is fused with the carbon atom far away from Y on the middle 6-membered ring;
more preferably, Ring A2 is 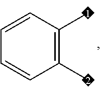
wherein Carbon atom No. 1 is fused with the carbon atom adjacent to Y on the middle 6-membered ring, and Carbon atom No. 2 is fused with the carbon atom far away from Y on the middle 6-membered ring;
Y is selected from —O—, —S—, —CR^{a}R^{b}—;
R^{a} and R^{b} are each independently selected from hydrogen and C₁-C₆ alkyl;
preferably, R^{a} and R^{b} are each independently selected from hydrogen and methyl;
preferably, Y is selected from —O—, —S—, —CH₂—, 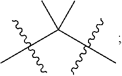 [SMILES: *C(*)(C)C]
more preferably, Y is selected from —O—, —S—;
n1 and n2 are each independently selected from 0 and 1, and n1 and n2 are not 0 at the same time;
preferably, n1 is 0 and n2 is 1;
preferably, 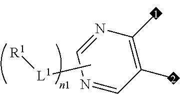
is selected from the group consisting of 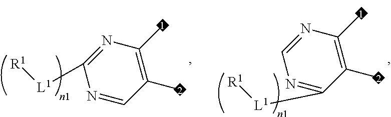
wherein Carbon atom No. 1 is bonded to the central spiro carbon atom, and Carbon atom No. 2 is bonded to a carbon atom of the aromatic ring on the right;
preferably, 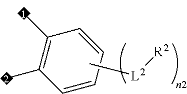
is selected from the group consisting of 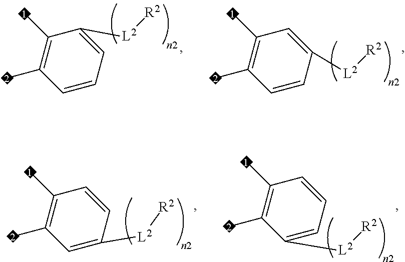
wherein Carbon atom No. 1 is bonded to the central spiro carbon atom, and Carbon atom No. 2 is bonded to a carbon atom of the aromatic ring on the left;
L¹ and L² are each independently selected from the group consisting of a direct bond and phenylene;
preferably, L¹ and L² are direct bonds;
R¹ and R² are each independently selected from diazaphenanthrolinyl, and the diazaphenanthrolinyl is optionally substituted by one or two (preferably two) R^{R}, and R^{R} is phenyl;
preferably, R¹ and R² are each independently selected from diazaphenanthrolinyl;
more preferably, R¹ and R² are each independently selected from the group consisting 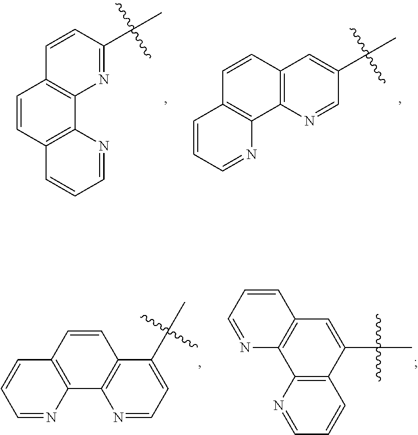 [SMILES: *c1ccc2ccc3cccnc3c2n1 ; *c1cnc2c(ccc3cccnc32)c1 ; *c1ccnc2c1ccc1cccnc12 ; *c1cc2cccnc2c2ncccc12]
most preferably, R¹-L¹, R²-L²- are each independently selected from the group consisting of:

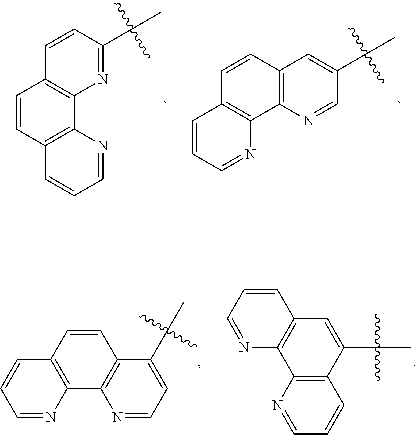 [SMILES: *c1ccc2ccc3cccnc3c2n1 ; *c1cnc2c(ccc3cccnc32)c1 ; *c1ccnc2c1ccc1cccnc12 ; *c1cc2cccnc2c2ncccc12]

11. The compound, or a stereoisomer or tautomer thereof according to claim 8, wherein the compound has the structure represented by Formula I-3, 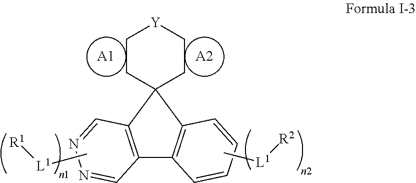
wherein:
Ring A1 is selected from benzene ring and naphthalene ring;
preferably, Ring A1 is selected from the group consisting of 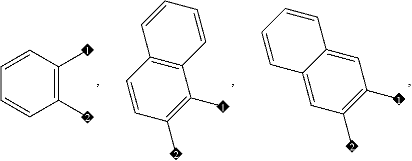
wherein Carbon atom No. 1 is fused with the carbon atom adjacent to Y on the middle 6-membered ring, and Carbon atom No. 2 is fused with the carbon atom far away from Y on the middle 6-membered ring;
more preferably, Ring A1 is 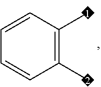
wherein Carbon atom No. 1 is fused with the carbon atom adjacent to Y on the middle 6-membered ring, and Carbon atom No. 2 is fused with the carbon atom far away from Y on the middle 6-membered ring;
Ring A2 is selected from benzene ring and naphthalene ring;
preferably, Ring A2 is selected from the group consisting of 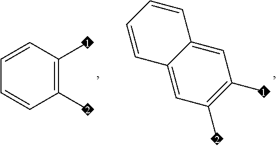
wherein Carbon atom No. 1 is fused with the carbon atom adjacent to Y on the middle 6-membered ring, and Carbon atom No. 2 is fused with the carbon atom far away from Y on the middle 6-membered ring;
more preferably, Ring A2 is 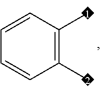
wherein Carbon atom No. 1 is fused with the carbon atom adjacent to Y on the middle 6-membered ring, and Carbon atom No. 2 is fused with the carbon atom far away from Y on the middle 6-membered ring;
Y is selected from —O—, —S—, —CR^{a}R^{b}—;
R^{a} and R^{b} are each independently selected from hydrogen and C₁-C₆ alkyl;
preferably, R^{a} and R^{b} are each independently selected from hydrogen and methyl;
preferably, Y is selected from —O—, —S—, —CH₂—, 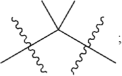 [SMILES: *C(*)(C)C]
more preferably, Y is selected from —O—, —S—;
n1 and n2 are each independently selected from 0 and 1, and n1 and n2 are not 0 at the same time;
preferably, n1 is 0 and n2 is 1;
preferably, 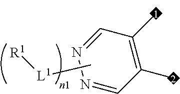
is selected from 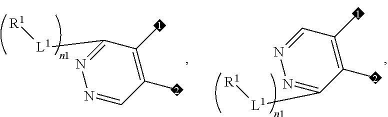
wherein Carbon atom No. 1 is bonded to the central spiro carbon atom, and Carbon atom No. 2 is bonded to a carbon atom of the aromatic ring on the right;
preferably, 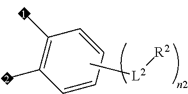
is selected from the group consisting of 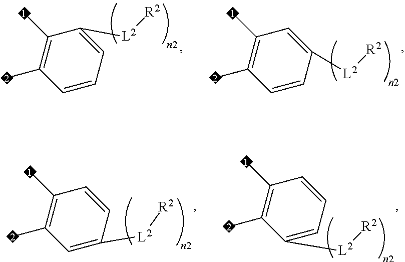
wherein Carbon atom No. 1 is bonded to the central spiro carbon atom, and Carbon atom No. 2 is bonded to a carbon atom of the aromatic ring on the left;
L¹ and L² are each independently selected from the group consisting of a direct bond and phenylene;
preferably, L¹ and L² are direct bonds;
R¹ and R² are each independently selected from the group consisting of diazaphenanthrolinyl, and the diazaphenanthrolinyl is optionally substituted by one or two (preferably two) R^{R}, and R^{R} is phenyl;
preferably, R¹ and R² are each independently selected from diazaphenanthrolinyl;
more preferably, R¹ and R² are each independently selected from the group consisting of: 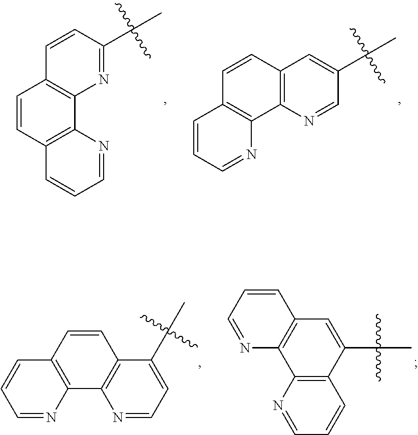 [SMILES: *c1ccc2ccc3cccnc3c2n1 ; *c1cnc2c(ccc3cccnc32)c1 ; *c1ccnc2c1ccc1cccnc12 ; *c1cc2cccnc2c2ncccc12]
most preferably, R¹-L¹-, R²-L²- are each independently selected from the group

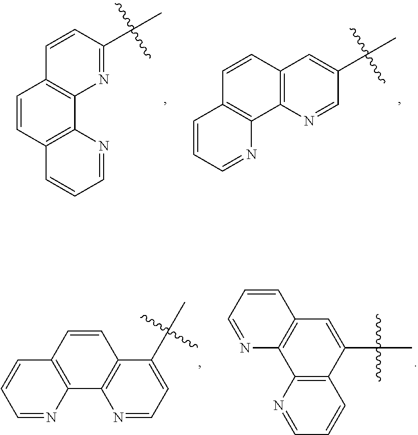 [SMILES: *c1ccc2ccc3cccnc3c2n1 ; *c1cnc2c(ccc3cccnc32)c1 ; *c1ccnc2c1ccc1cccnc12 ; *c1cc2cccnc2c2ncccc12]

12. The compound, or a stereoisomer or tautomer thereof according to claim 1, wherein the compound has a structure represented by Formula II, 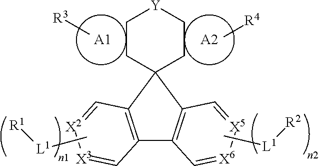
Formula II
X² and X³ are each independently selected from CH and N, and at least one of X² and X³ is N;
X⁵ and X⁶ are each independently selected from CH and N, and at least one of X⁵ and X⁶ is N;
n1 and n2 are each independently selected from 0 and 1, and n1 and n2 are not 0 at the same time;
preferably, 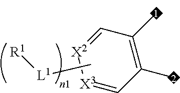
is selected from the group consisting of 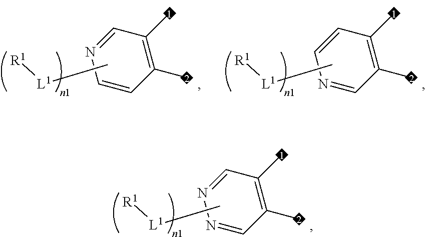
wherein Carbon atom No. 1 is bonded to the central spiro carbon atom, and Carbon atom No. 2 is bonded to a carbon atom of the aromatic ring on the right;
more preferably, 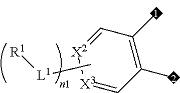
is selected from the group consisting of: 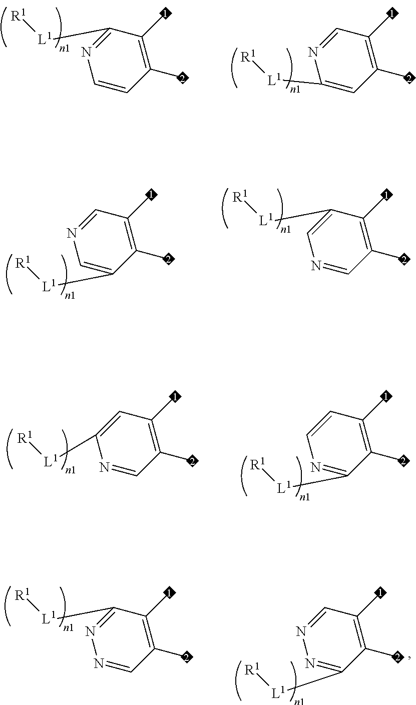
wherein, Carbon atom No. 1 is bonded to the central spiro carbon atom, and Carbon atom No. 2 is bonded to a carbon atom of the aromatic ring on the right;
preferably, 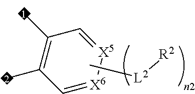
is selected from 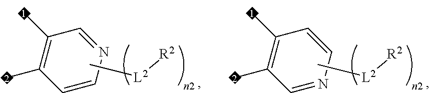
wherein Carbon atom No. 1 is bonded to the central spiro carbon atom, and Carbon atom No. 2 is bonded to a carbon atom of the aromatic ring on the left;
more preferably, 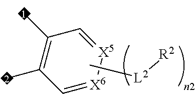
is selected from the group consisting of: 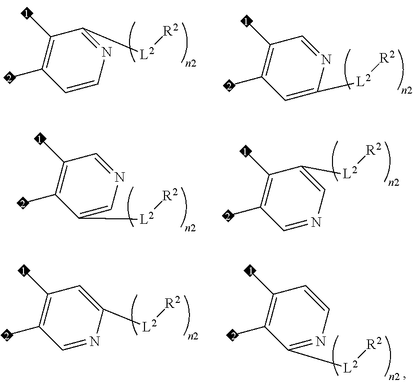
wherein, Carbon atom No. 1 is bonded to the central spiro carbon atom, and Carbon atom No. 2 is bonded to a carbon atom of the aromatic ring on the left;
Ring A1 and Ring A2 are each independently selected from benzene ring and naphthalene ring;
preferably, Ring A1 and Ring A2 are benzene rings;
Y is selected from —O—, —S—, —CR^{a}R^{b}—;
R^{a} and R^{b} are each independently selected from hydrogen and C₁-C₆ alkyl;
preferably, R^{a} and R^{b} are each independently selected from C₁-C₆ alkyl;
more preferably, R^{a} and R^{b} are methyl groups;
preferably, Y is selected from —O—, —S—, 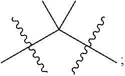 [SMILES: *C(*)(C)C]
more preferably, Y is selected from —O—, —S—;
L¹ and L² are each independently selected from the group consisting of a direct bond, phenylene, and naphthylene, and the phenylene and naphthylene are each independently optionally substituted by one R^{L};
preferably, L¹ and L² are each independently selected from the group consisting of a direct bond and phenylene, and the phenylene is optionally substituted by one R^{L};
more preferably, L¹ and L² are each independently selected from the group consisting of a direct bond, 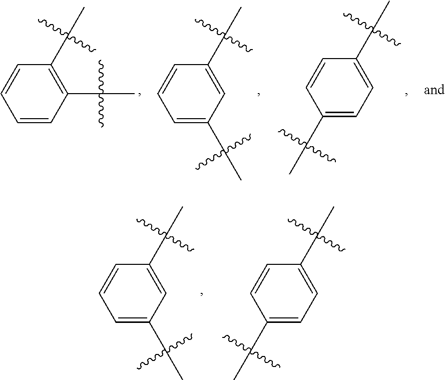 [SMILES: *c1ccccc1* ; *c1cccc(*)c1 ; *c1ccc(*)cc1 ; *c1cccc(*)c1 ; *c1ccc(*)cc1]
are each independently optionally substituted by one R^{L};
R^{L} is selected from benzoxazolyl and benzothiazolyl;
preferably, R^{L} is benzoxazolyl;
more preferably, R^{L} is selected from 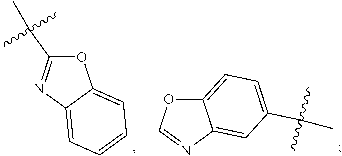
most preferably, L¹ and L² are each independently selected from the group consisting of:
a direct bond, 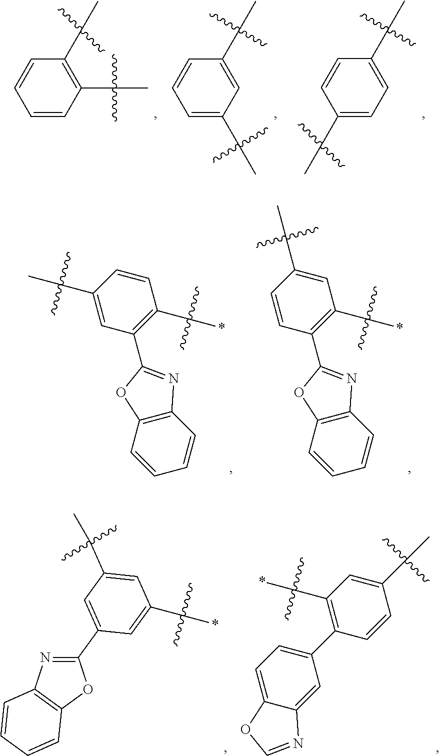
wherein, the bond with marker * is bonded to the parent ring, and the bond without marker * is bonded to R¹ or R²;
R¹ and R² are each independently selected from the group consisting of diazaphenanthrolinyl, pyridinyl, pyrimidinyl, triazinyl, benzoxazolyl, benzothiazolyl, and the diazaphenanthrolinyl, pyridinyl, pyrimidinyl, triazinyl, benzoxazolyl and benzothiazolyl are each independently optionally substituted by one or two (preferably two) R^{R};
preferably, R¹ and R² are each independently selected from the group consisting of diazaphenanthrolinyl, pyridinyl, pyrimidinyl, triazinyl, benzoxazolyl, benzothiazolyl, and the pyridinyl, pyrimidinyl, triazinyl, benzoxazolyl are each independently optionally substituted by one or two (preferably two) R^{R};
more preferably, R¹ and R² are each independently selected from o-diazaphenanthrolinyl, pyridinyl, pyrimidinyl, 1,3,5-triazinyl, benzoxazolyl, and the pyridinyl, pyrimidinyl, 1,3,5-triazinyl, benzoxazolyl are each independently optionally substituted by one or two (preferably two) R^{R};
further preferably, R¹ and R² are each independently selected from the group consisting of 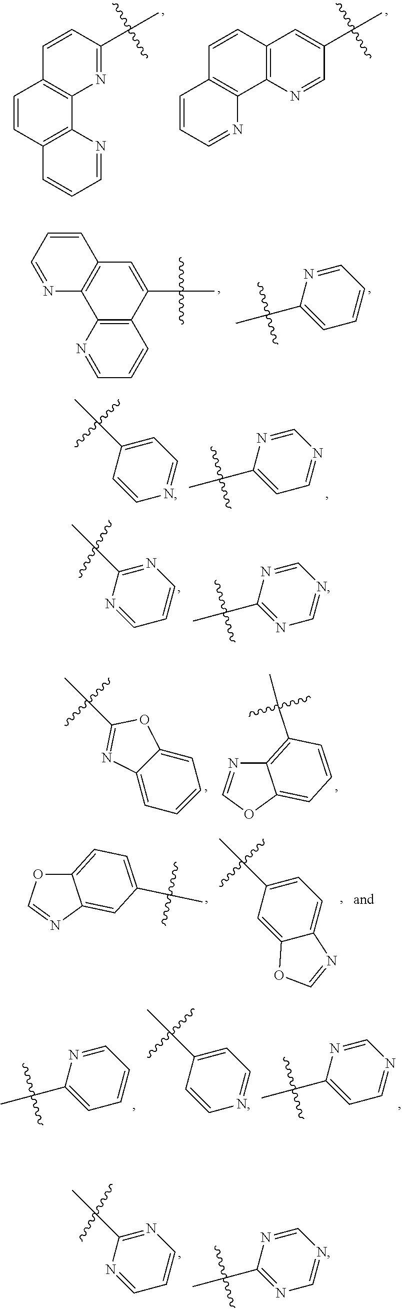 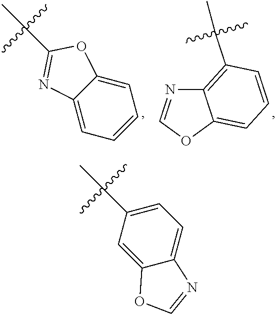
are each independently optionally substituted by one or two (preferably two) R^{R};
R^{R} is selected from the group consisting of phenyl, biphenyl, naphthyl, and fluorenyl, and the phenyl, biphenyl, naphthyl, and fluorenyl are each independently optionally substituted by two C₁-C₆ alkyl groups;
preferably, R^{R} is selected from the group consisting of phenyl, biphenyl, naphthyl, and fluorenyl, and the fluorenyl is optionally substituted by two C₁-C₆ alkyl groups;
more preferably, R^{R} is selected from the group consisting of phenyl, biphenyl, naphthyl, and fluorenyl, and the fluorenyl is optionally substituted by two methyl groups;
most preferably, R^{R} is selected from the group consisting of 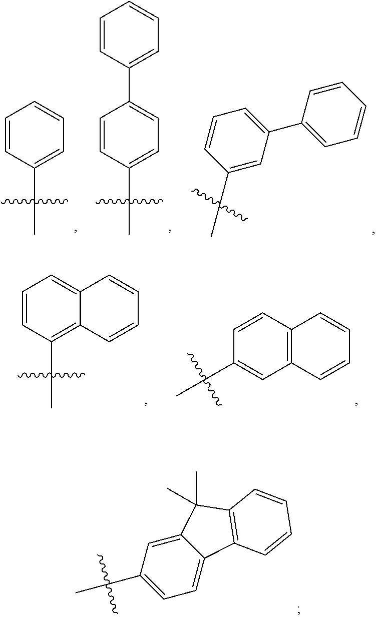
most preferably, R¹ and R² are each independently selected from the group consisting 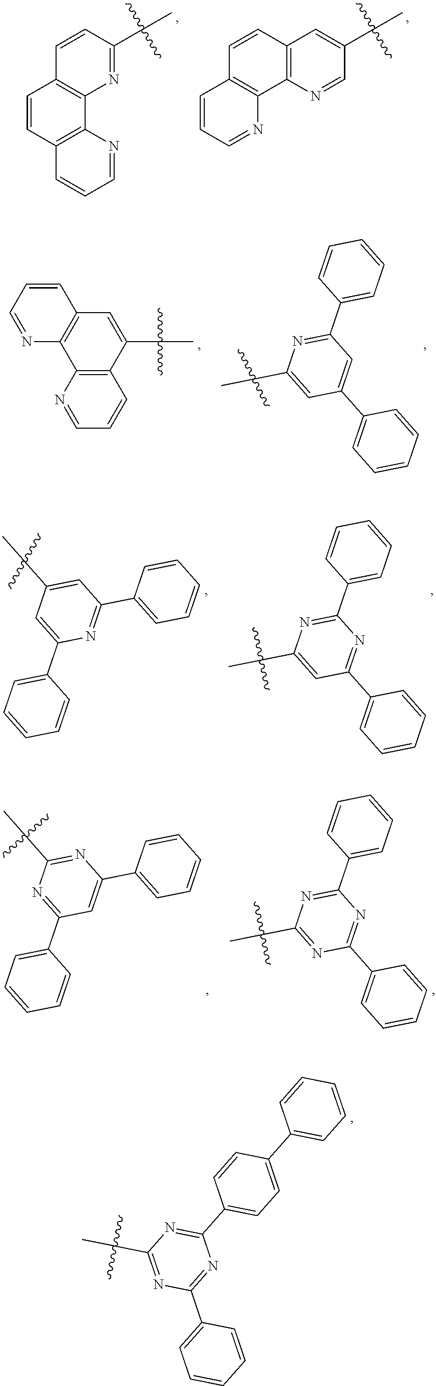 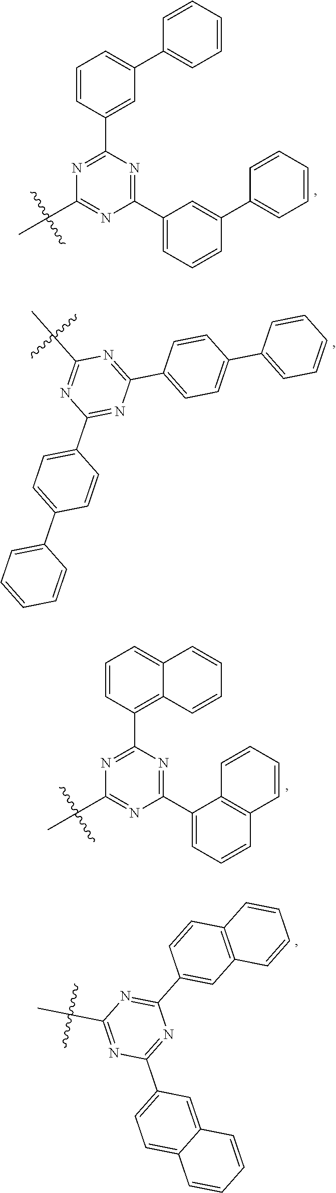 [SMILES: *c1nc(-c2cccc(-c3ccccc3)c2)nc(-c2cccc(-c3ccccc3)c2)n1 ; *c1nc(-c2ccc(-c3ccccc3)cc2)nc(-c2ccc(-c3ccccc3)cc2)n1 ; *c1nc(-c2cccc3ccccc23)nc(-c2cccc3ccccc23)n1 ; *c1nc(-c2ccc3ccccc3c2)nc(-c2ccc3ccccc3c2)n1] 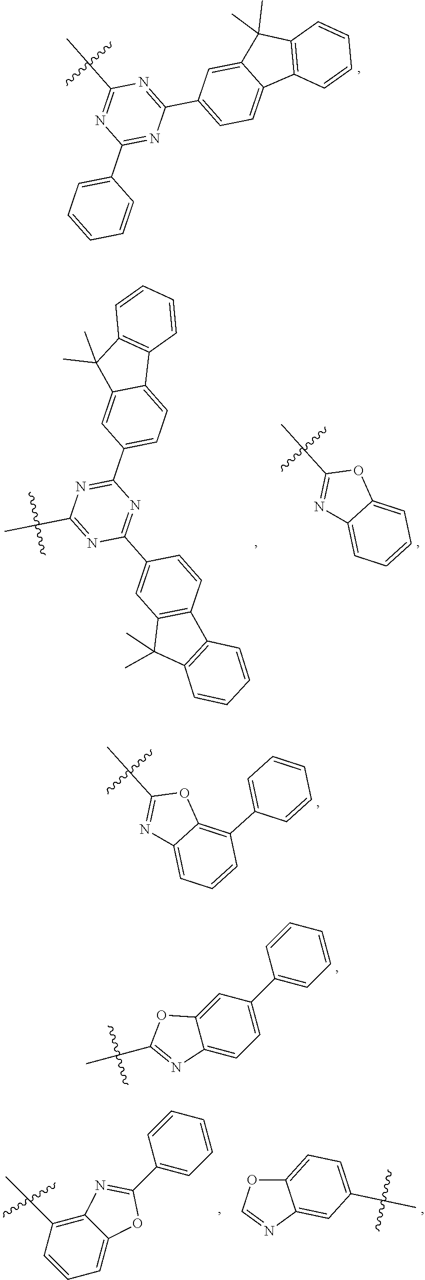 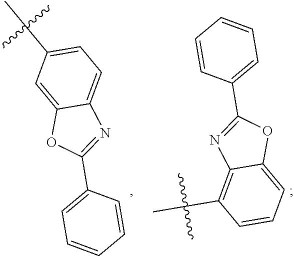 [SMILES: *c1ccc2nc(-c3ccccc3)oc2c1 ; *c1cccc2oc(-c3ccccc3)nc12]
most preferably, R¹-L¹, R²-L²- are each independently selected from the group consisting of: 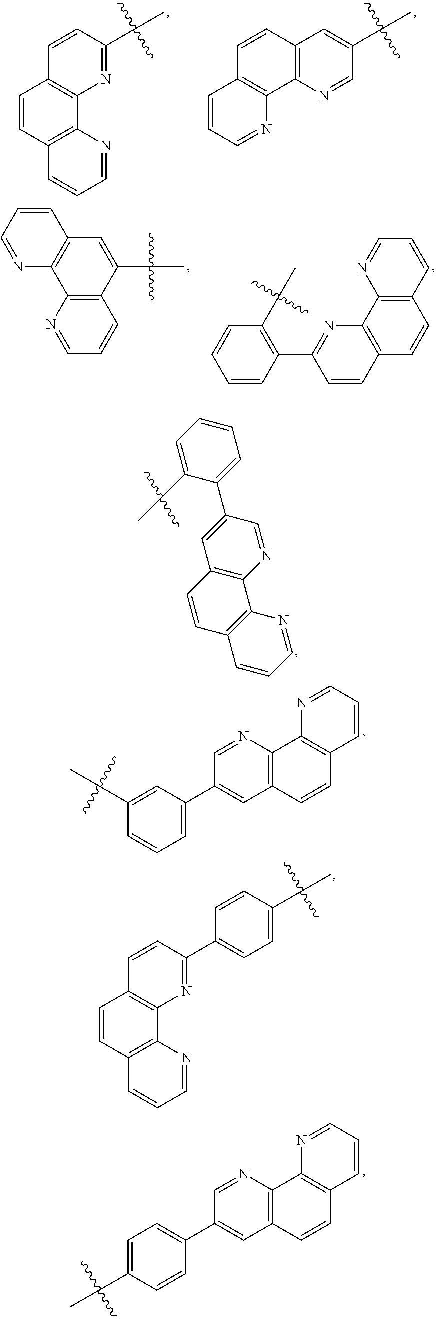 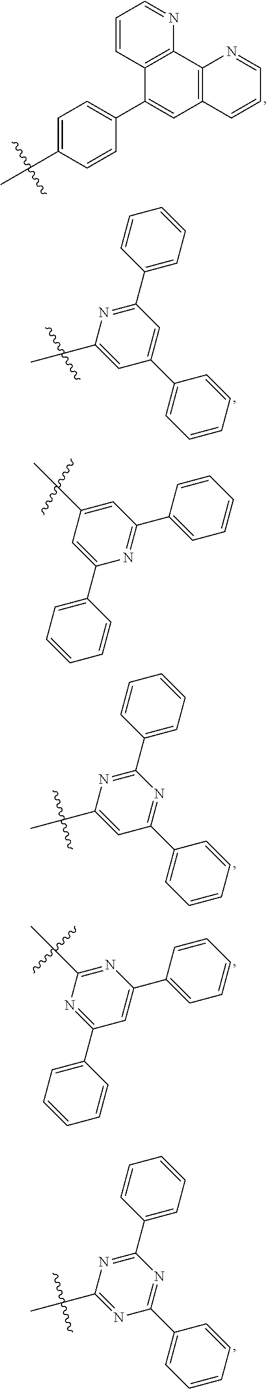 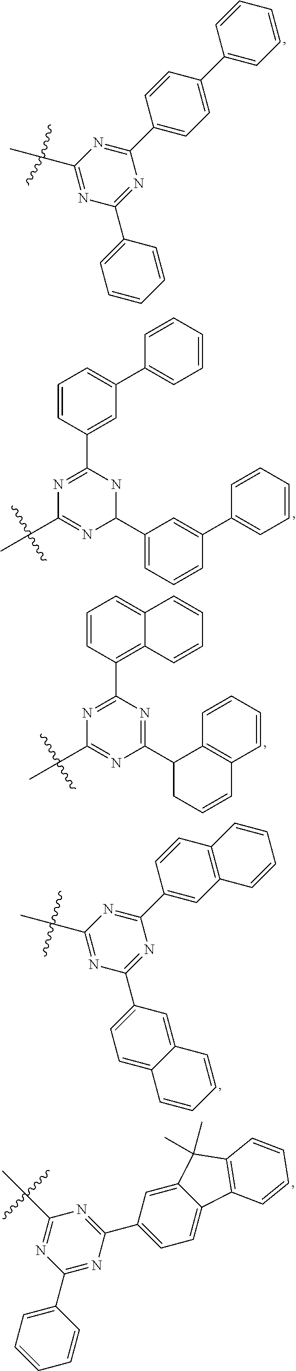 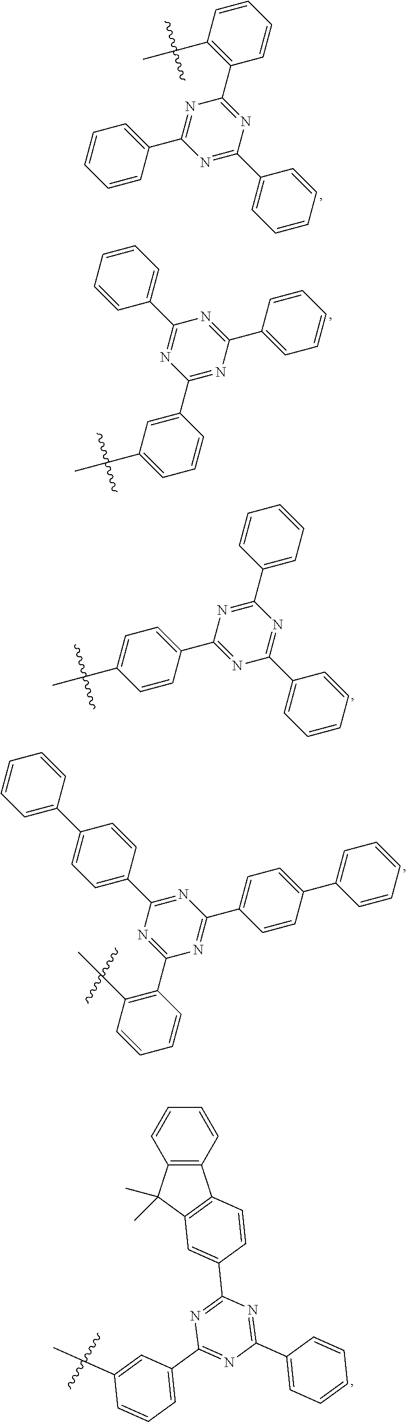 [SMILES: *c1ccccc1-c1nc(-c2ccccc2)nc(-c2ccccc2)n1 ; *c1cccc(-c2nc(-c3ccccc3)nc(-c3ccccc3)n2)c1 ; *c1ccc(-c2nc(-c3ccccc3)nc(-c3ccccc3)n2)cc1 ; *c1ccccc1-c1nc(-c2ccc(-c3ccccc3)cc2)nc(-c2ccc(-c3ccccc3)cc2)n1 ; *c1cccc(-c2nc(-c3ccccc3)nc(-c3ccc4c(c3)C(C)(C)c3ccccc3-4)n2)c1] 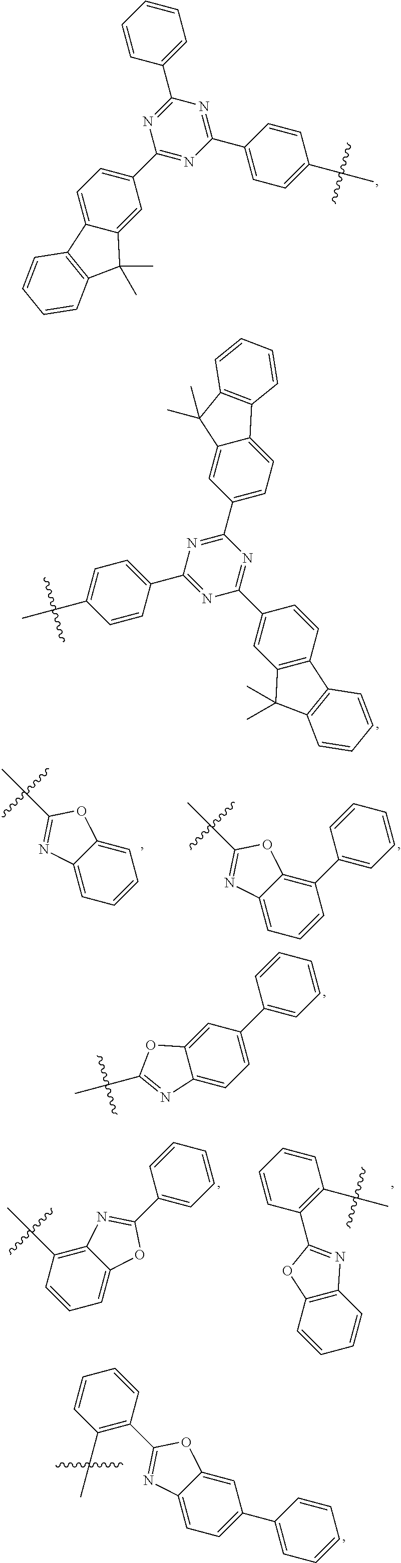 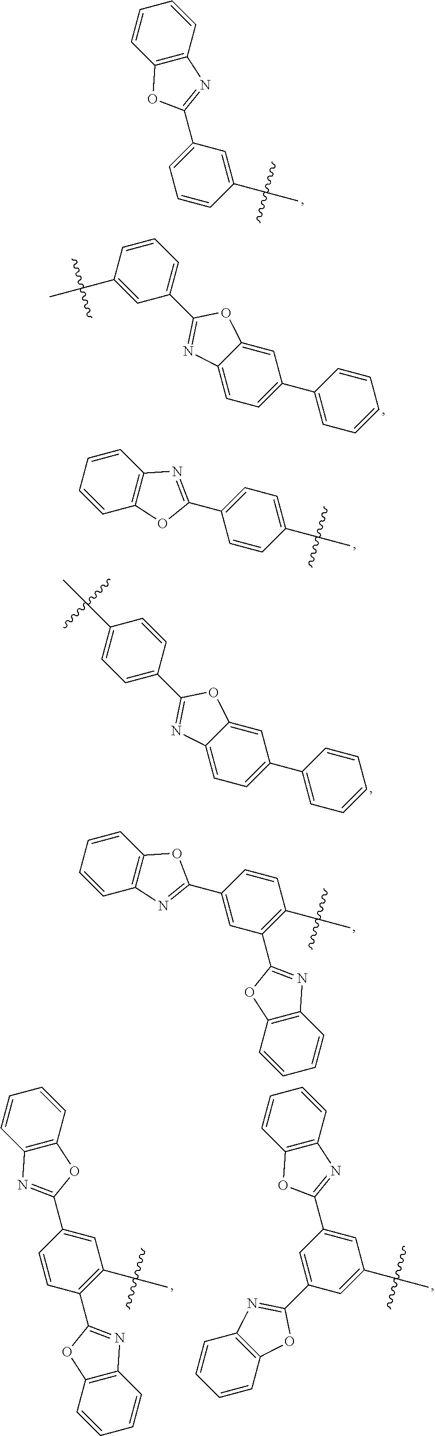 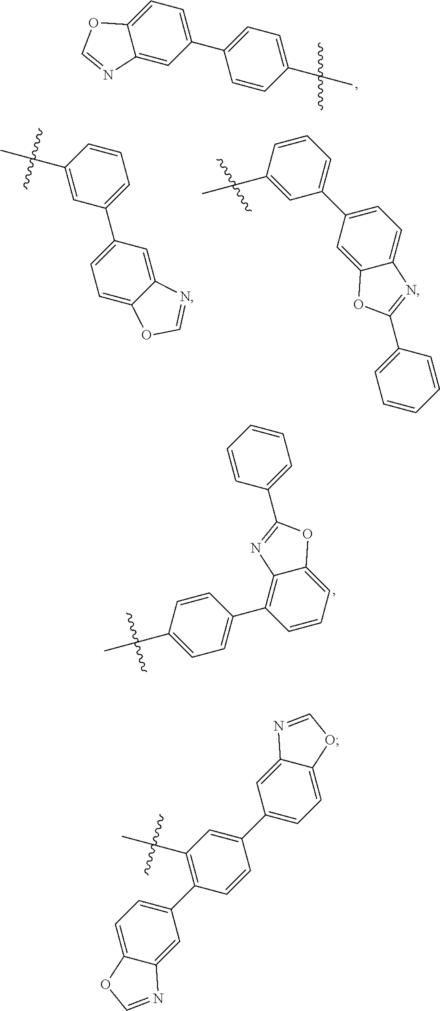 [SMILES: *c1ccc(-c2ccc3ocnc3c2)cc1 ; *c1cccc(-c2ccc3ocnc3c2)c1 ; *c1cccc(-c2ccc3nc(-c4ccccc4)oc3c2)c1 ; *c1ccc(-c2cccc3oc(-c4ccccc4)nc23)cc1 ; *c1cc(-c2ccc3ocnc3c2)ccc1-c1ccc2ocnc2c1]
R³ and R⁴ are each independently selected from hydrogen and C₁-C₆ alkyl;
preferably, R³ and R⁴ are hydrogen.

13. The compound, or a stereoisomer or tautomer thereof according to claim 12, wherein the compound has the structure represented by Formula II-1, 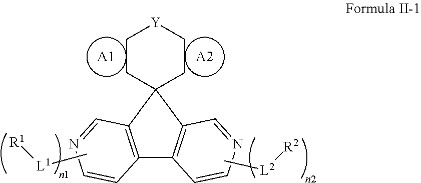
wherein:
Ring A1 and Ring A2 are each independently selected from benzene ring and naphthalene ring;
preferably, Ring A1 and Ring A2 are benzene rings;
Y is selected from —O—, —S—, —CR^{a}R^{b}—;
R^{a} and R^{b} are each independently selected from hydrogen and C₁-C₆ alkyl;
preferably, R^{a} and R^{b} are each independently selected from C₁-C₆ alkyl;
more preferably, R^{a} and R^{b} are methyl groups;
preferably, Y is selected from —O—, —S—, 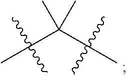 [SMILES: *C(*)(C)C]
more preferably, Y is selected from —O—, —S—;
n1 and n2 are each independently selected from 0 and 1, and n1 and n2 are not 0 at the same time;
preferably, 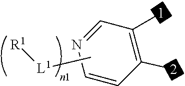
is selected from the group consisting of 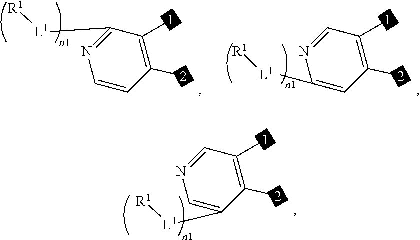
wherein Carbon atom No. 1 is bonded to the central spiro carbon atom, and Carbon atom No. 2 is bonded to a carbon atom of the aromatic ring on the right;
preferably, 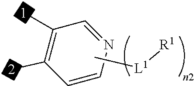
is selected from the group consisting of 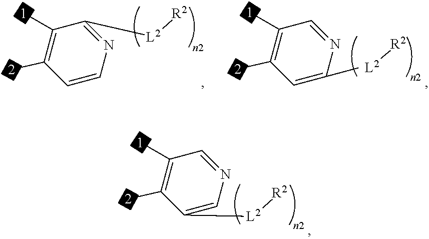
wherein, Carbon atom No. 1 is bonded to the central spiro carbon atom, and Carbon atom No. 2 is bonded to a carbon atom of the aromatic ring on the left;
L¹ and L² are each independently selected from the group consisting of a direct bond and phenylene, and the phenylene is optionally substituted by one R^{L}, and R^{L} is benzoxazolyl;
preferably, L¹ and L² are each independently selected from the group consisting of a direct bond, 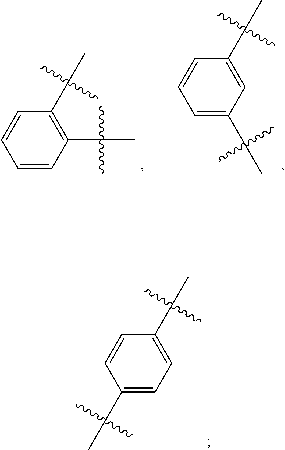 [SMILES: *c1ccccc1* ; *c1cccc(*)c1 ; *c1ccc(*)cc1]
R¹ and R² are each independently selected from the group consisting of diazaphenanthrolinyl, pyridinyl, triazinyl, benzoxazolyl, and benzothiazolyl, and the diazaphenanthrolinyl, pyridinyl, triazinyl, benzoxazolyl and benzothiazolyl are each independently optionally substituted by one or two (preferably two) R^{R};
preferably, R¹ and R² are each independently selected from the group consisting of diazaphenanthrolinyl, pyridinyl, triazinyl, and benzoxazolyl, and the pyridinyl, triazinyl, and benzoxazolyl are each independently optionally substituted by one or two (preferably two) R^{R};
more preferably, R¹ and R² are each independently selected from the group consisting of o-diazaphenanthrolinyl, pyridinyl, 1,3,5-triazinyl, and benzoxazolyl, and the pyridinyl, 1,3,5-triazinyl, and benzoxazolyl are each independently optionally substituted by one or two (preferably two) R^{R};
further preferably, R¹ and R² are each independently selected from the group consisting of 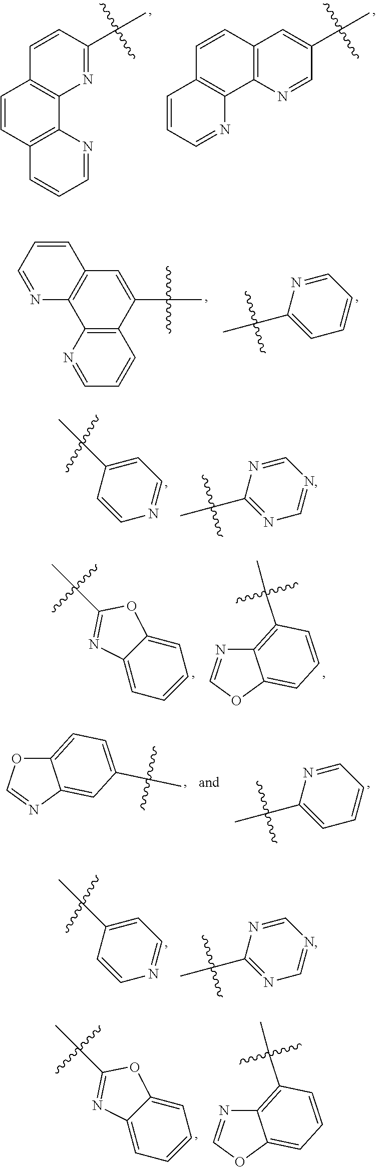
are each independently optionally substituted by one or two (preferably two) R^{R};
R^{R} is selected from the group consisting of phenyl, biphenyl, and fluorenyl, and the phenyl, biphenyl, and fluorenyl are each independently optionally substituted by two C₁-C₆ alkyl groups;
preferably, R^{R} is selected from the group consisting of phenyl, biphenyl, and fluorenyl, and the fluorenyl is optionally substituted by two C₁-C₆ alkyl groups;
more preferably, R^{R} is selected from the group consisting of phenyl, biphenyl, and fluorenyl, and the fluorenyl is optionally substituted by two methyl groups;
most preferably, R^{R} is selected from the group consisting of 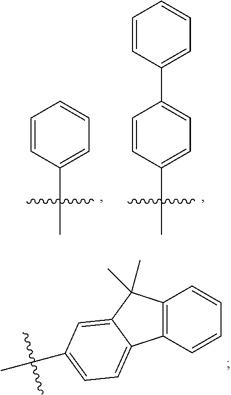
most preferably, R¹ and R² are each independently selected from the group consisting of: 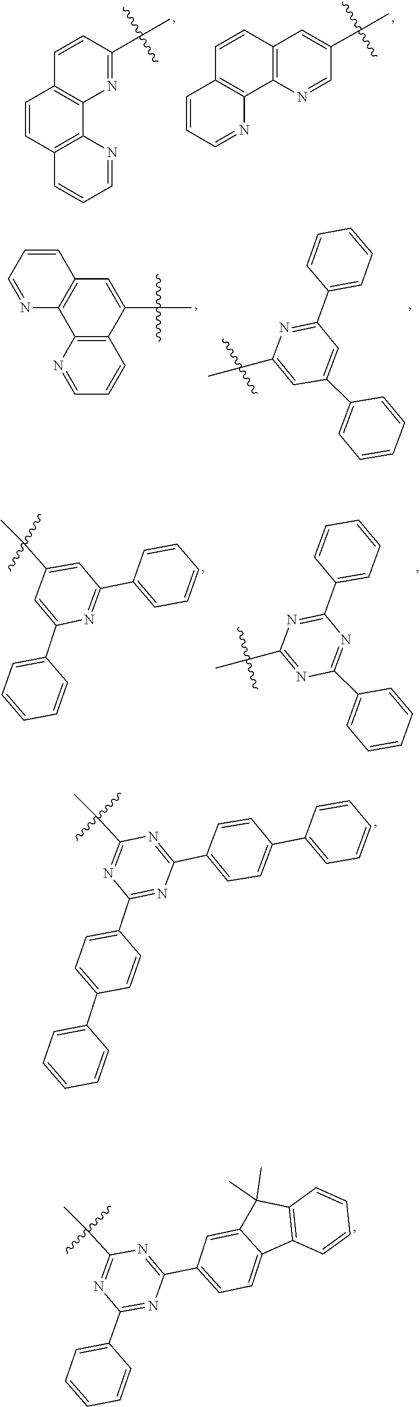 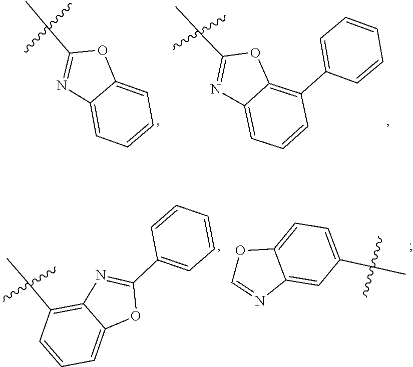
most preferably, R¹-L¹-, R²-L²- are each independently selected from the group

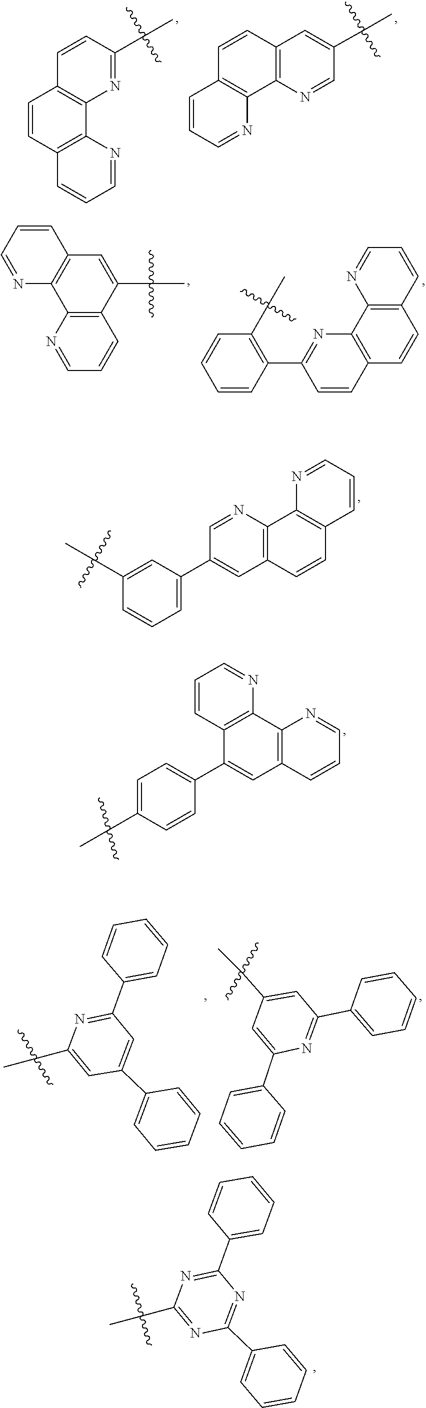

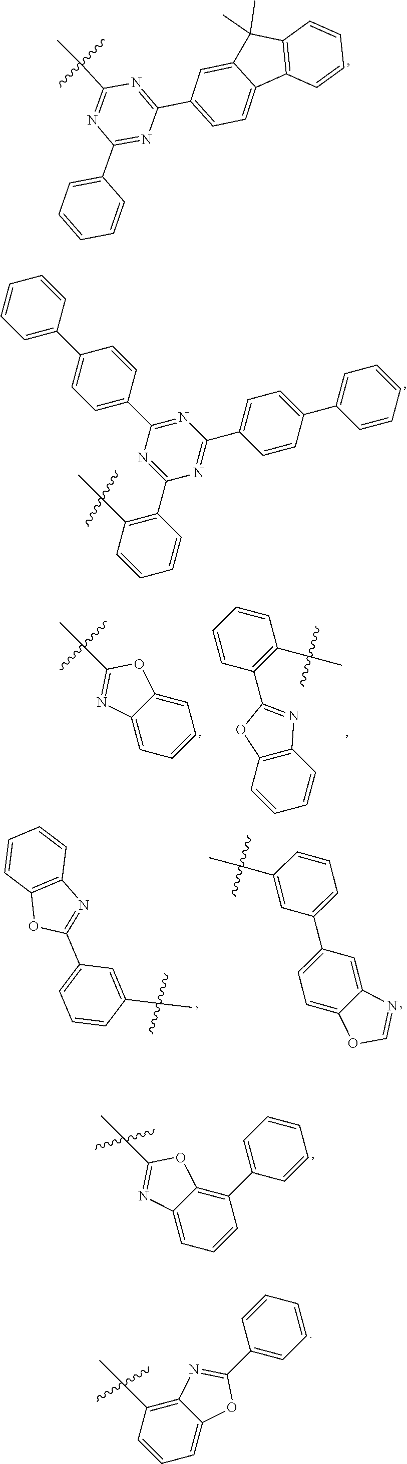

14. The compound, or a stereoisomer or tautomer thereof according to claim 12, wherein the compound has the structure represented by Formula II-2, 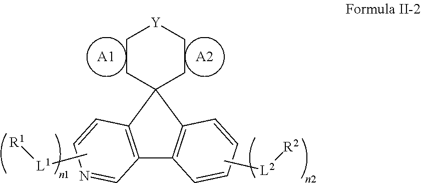
wherein:
Ring A1 and Ring A2 are each independently selected from benzene ring and naphthalene ring;
preferably, Ring A1 and Ring A2 are benzene rings;
Y is selected from —O—, —S—, —CR^{a}R^{b}—;
R^{a} and R^{b} are each independently selected from hydrogen and C₁-C₆ alkyl;
preferably, R^{a} and R^{b} are each independently selected from C₁-C₆ alkyl;
more preferably, R^{a} and R^{b} are methyl groups;
preferably, Y is selected from —O—, —S—, 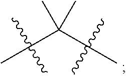 [SMILES: *C(*)(C)C]
more preferably, Y is selected from —O—, —S—;
n1 and n2 are each independently selected from 0 and 1, and n1 and n2 are not 0 at the same time;
preferably, 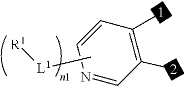
is selected from the group consisting of 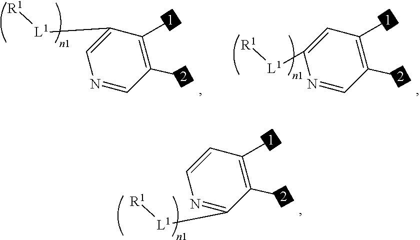
wherein Carbon atom No. 1 is bonded to the central spiro carbon atom, and Carbon atom No. 2 is bonded to a carbon atom of the aromatic ring on the right;
preferably, 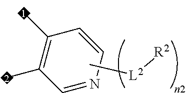
is selected from the group consisting of 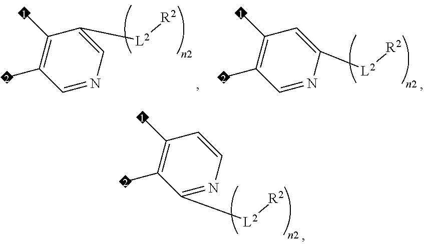
wherein, Carbon atom No. 1 is bonded to the central spiro carbon atom, and Carbon atom No. 2 is bonded to a carbon atom of the aromatic ring on the left;
L¹ and L² are each independently selected from the group consisting of a direct bond and phenylene, and the phenylene is optionally substituted by one R^{L}, and R^{L} is benzoxazolyl;
preferably, L¹ and L² are each independently selected from the group consisting of a direct bond, 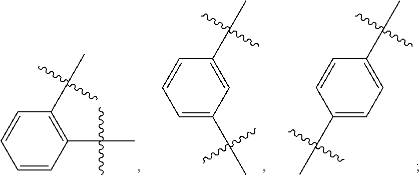 [SMILES: *c1ccccc1* ; *c1cccc(*)c1 ; *c1ccc(*)cc1]
R¹ and R² are each independently selected from the group consisting of diazaphenanthrolinyl, pyridinyl, pyrimidinyl, triazinyl, benzoxazolyl, benzothiazolyl, and the diazaphenanthrolinyl, pyridinyl, pyrimidinyl, triazinyl, benzoxazolyl and benzothiazolyl are each independently optionally substituted by one or two (preferably two) R^{R};
preferably, R¹ and R² are each independently selected from the group consisting of diazaphenanthrolinyl, pyridinyl, pyrimidinyl, triazinyl, and benzoxazolyl, and the pyridinyl, pyrimidinyl, triazinyl, benzoxazolyl are each independently optionally substituted by one or two (preferably two) R^{R};
more preferably, R¹ and R² are each independently selected from o-diazaphenanthrolinyl, pyridinyl, pyrimidinyl, 1,3,5-triazinyl, benzoxazolyl, and the pyridinyl, pyrimidinyl, 1,3,5-triazinyl, benzoxazolyl are each independently optionally substituted by one or two (preferably two) R^{R};
further preferably, R¹ and R² are each independently selected from the group consisting of 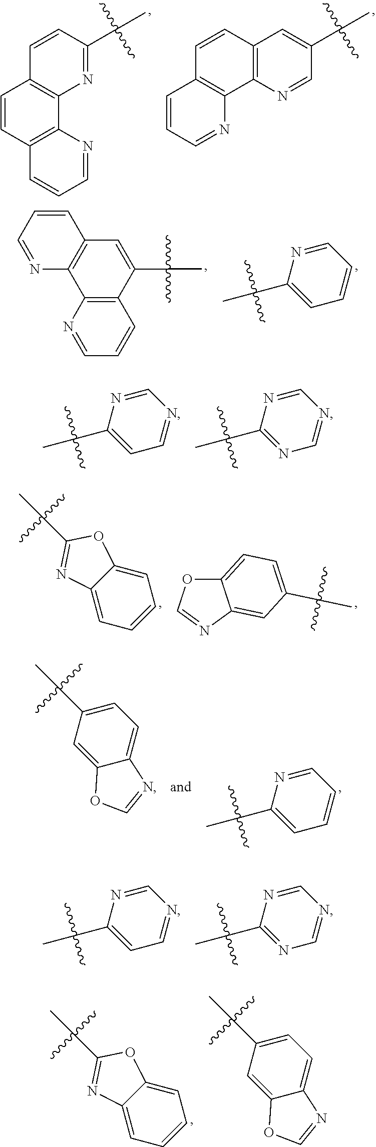
are each independently optionally substituted by one or two R^{R};
R^{R} is selected from the group consisting of phenyl, biphenyl, and fluorenyl, and the phenyl, biphenyl, and fluorenyl are each independently optionally substituted by two C₁-C₆ alkyl groups;
preferably, R^{R} is selected from the group consisting of phenyl, biphenyl, and fluorenyl, and the fluorenyl is optionally substituted by two C₁-C₆ alkyl groups;
more preferably, R^{R} is selected from the group consisting of phenyl, biphenyl, and fluorenyl, and the fluorenyl is optionally substituted by two methyl groups;
most preferably, R^{R} is selected from the group consisting of 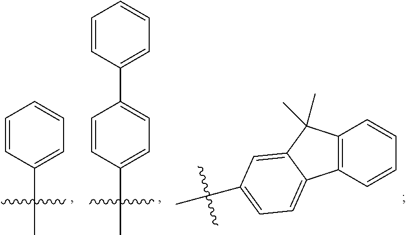
most preferably, R¹ and R² are each independently selected from the group consisting of: 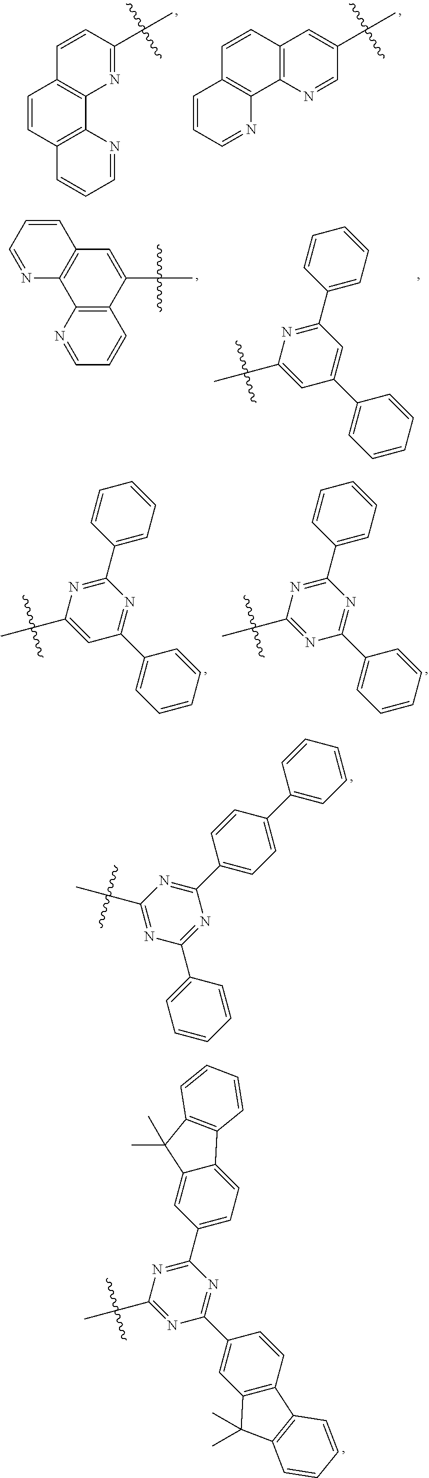 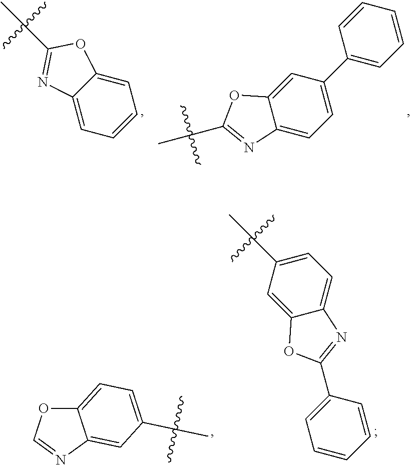 [SMILES: *c1nc2ccccc2o1 ; *c1nc2ccc(-c3ccccc3)cc2o1 ; *c1ccc2ocnc2c1 ; *c1ccc2nc(-c3ccccc3)oc2c1]
most preferably, R¹-L¹-, R²-L²- are each independently selected from the group consisting of:

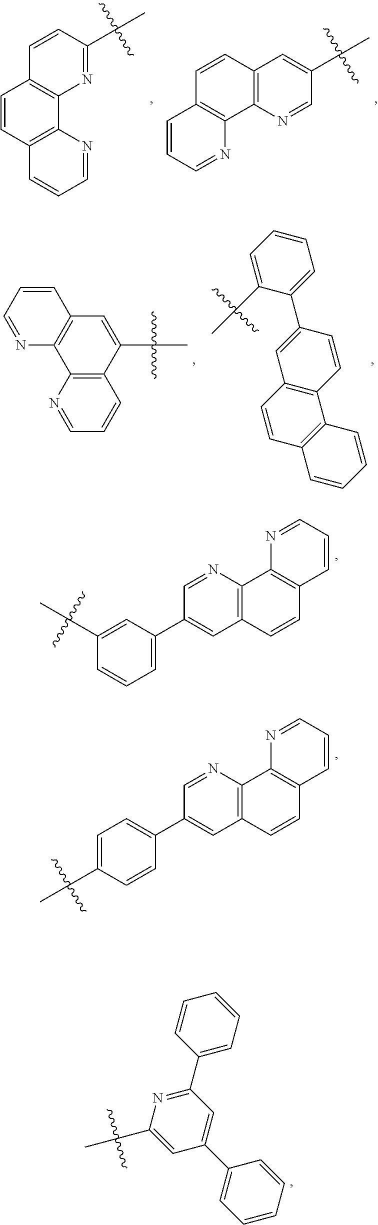

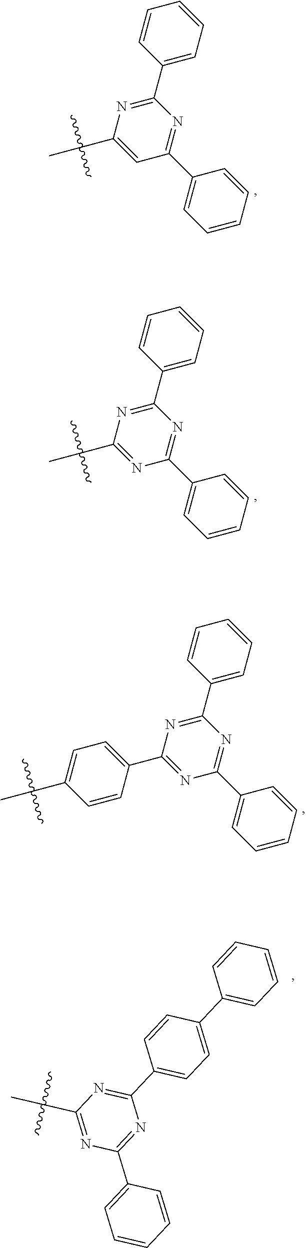 [SMILES: *c1cc(-c2ccccc2)nc(-c2ccccc2)n1 ; *c1nc(-c2ccccc2)nc(-c2ccccc2)n1 ; *c1ccc(-c2nc(-c3ccccc3)nc(-c3ccccc3)n2)cc1 ; *c1nc(-c2ccccc2)nc(-c2ccc(-c3ccccc3)cc2)n1]

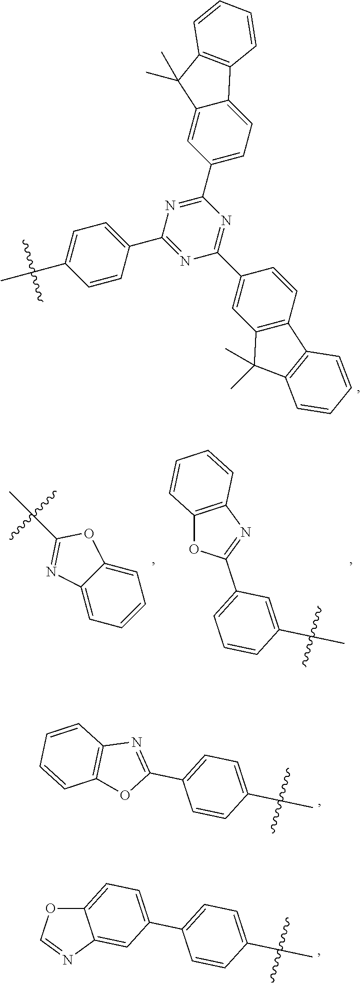 [SMILES: *c1ccc(-c2nc(-c3ccc4c(c3)C(C)(C)c3ccccc3-4)nc(-c3ccc4c(c3)C(C)(C)c3ccccc3-4)n2)cc1 ; *c1nc2ccccc2o1 ; *c1cccc(-c2nc3ccccc3o2)c1 ; *c1ccc(-c2nc3ccccc3o2)cc1 ; *c1ccc(-c2ccc3ocnc3c2)cc1]

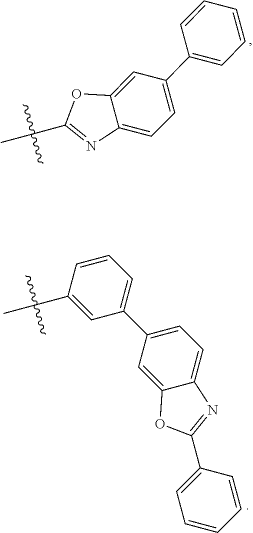 [SMILES: *c1nc2ccc(-c3ccccc3)cc2o1 ; *c1cccc(-c2ccc3nc(-c4ccccc4)oc3c2)c1]

15. The compound, or a stereoisomer or tautomer thereof according to claim 12, wherein the compound has the structure represented by Formula II-3, 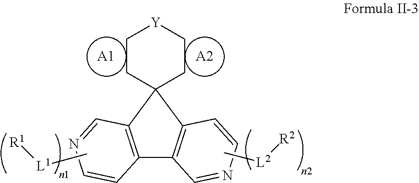
wherein:
Ring A1 and Ring A2 are each independently selected from benzene ring and naphthalene ring;
preferably, Ring A1 and Ring A2 are benzene rings;
Y is selected from —O—, —S—, —CR^{a}R^{b}—;
R^{a} and R^{b} are each independently selected from hydrogen and C₁-C₆ alkyl;
preferably, R^{a} and R^{b} are each independently selected from C₁-C₆ alkyl;
more preferably, R^{a} and R^{b} are methyl groups;
preferably, Y is selected from —O—, —S—, 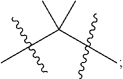 [SMILES: *C(*)(C)C]
more preferably, Y is selected from —O—, —S—;
n1 and n2 are each independently selected from 0 and 1, and n1 and n2 are not 0 at the same time;
preferably, 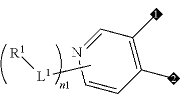
is selected from the group consisting of 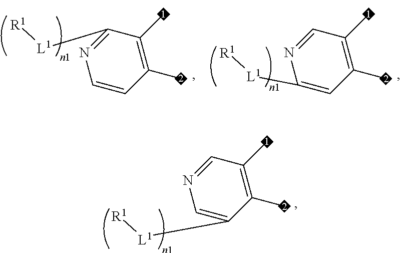
wherein, Carbon atom No. 1 is bonded to the central spiro carbon atom, and Carbon atom No. 2 is bonded to a carbon atom of the aromatic ring on the right;
preferably, 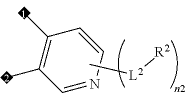
is selected from the group consisting of 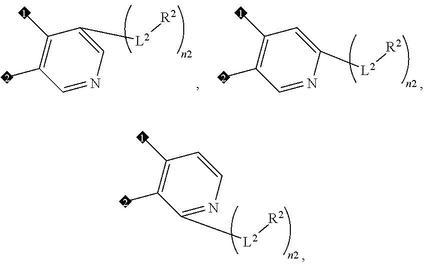
wherein, Carbon atom No. 1 is bonded to the central spiro carbon atom, and Carbon atom No. 2 is bonded to a carbon atom of the aromatic ring on the left;
L¹ and L² are each independently selected from the group consisting of a direct bond and phenylene, and the phenylene is optionally substituted by one R^{L}, and R^{L} is benzoxazolyl;
preferably, L¹ and L² are each independently selected from the group consisting of a direct bond 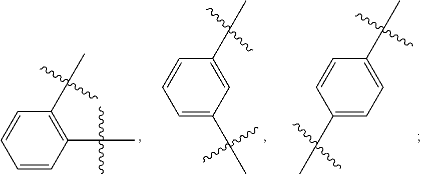 [SMILES: *c1ccccc1* ; *c1cccc(*)c1 ; *c1ccc(*)cc1]
R¹ and R² are each independently selected from the group consisting of diazaphenanthrolinyl, pyrimidinyl, triazinyl, benzoxazolyl, benzothiazolyl, and the diazaphenanthrolinyl, pyrimidinyl, triazinyl, benzoxazolyl and benzothiazolyl are each independently optionally substituted by one or two (preferably two) R^{R};
preferably, R¹ and R² are each independently selected from the group consisting of diazaphenanthrolinyl, pyrimidinyl, triazinyl, and benzoxazolyl, and the pyrimidinyl, triazinyl, and benzoxazolyl are each independently optionally substituted by one or two (preferably two) R^{R};
more preferably, R¹ and R² are each independently selected from o-diazaphenanthrolinyl, pyrimidinyl, 1,3,5-triazinyl, and benzoxazolyl, and the pyrimidinyl, 1,3,5-triazinyl, and benzoxazolyl are each independently optionally substituted by one or two (preferably two) R^{R};
further preferably, R¹ and R² are each independently selected from the group consisting of 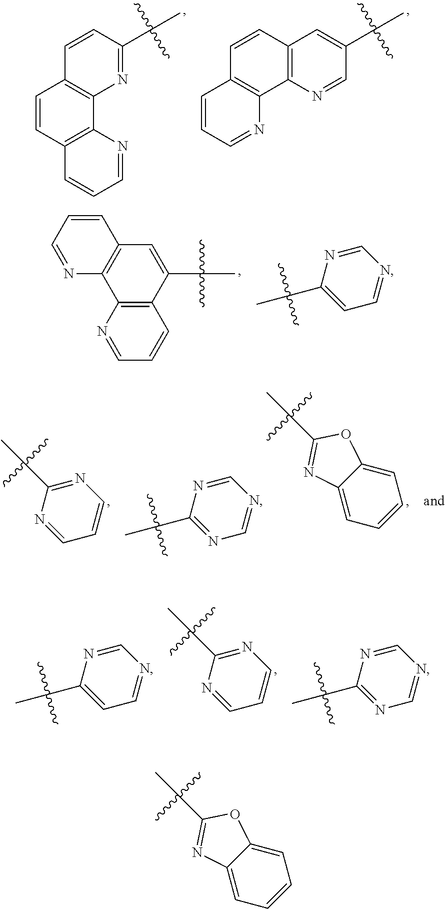
are each independently optionally substituted by one or two R^{R};
R^{R} is selected from phenyl and fluorenyl, and the phenyl and fluorenyl are each independently optionally substituted by two C₁-C₆ alkyl groups;
preferably, R^{R} is selected from phenyl and fluorenyl, and the fluorenyl is optionally substituted by two C₁-C₆ alkyl groups;
more preferably, R^{R} is selected from phenyl and fluorenyl, and the fluorenyl is optionally substituted by two methyl groups;
most preferably, R^{R} is selected from 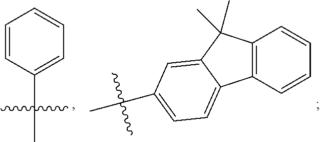 [SMILES: *c1ccccc1 ; *c1ccc2c(c1)C(C)(C)c1ccccc1-2]
most preferably, R¹ and R² are each independently selected from the group consisting 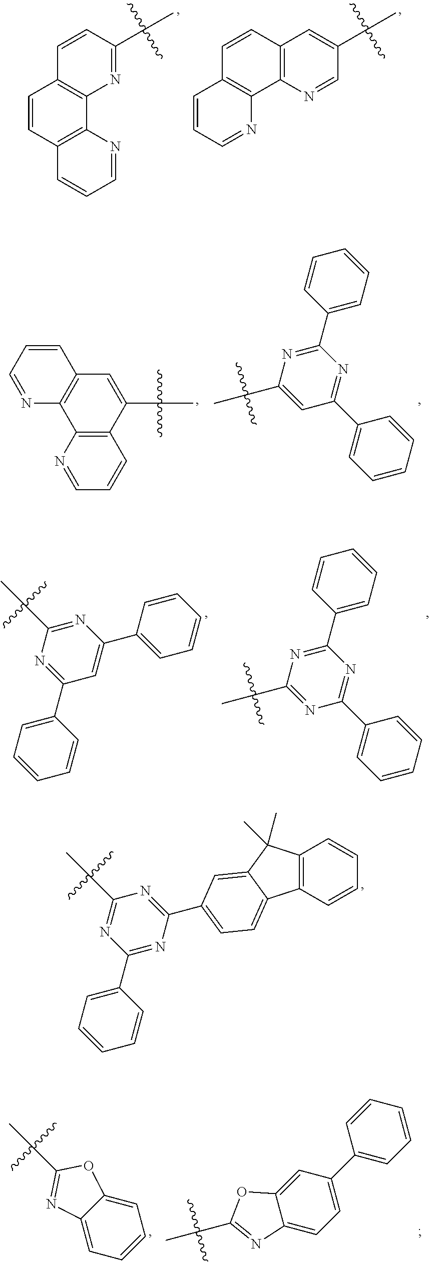
most preferably, R¹-L¹-, R²-L²- are each independently selected from the group consisting of:

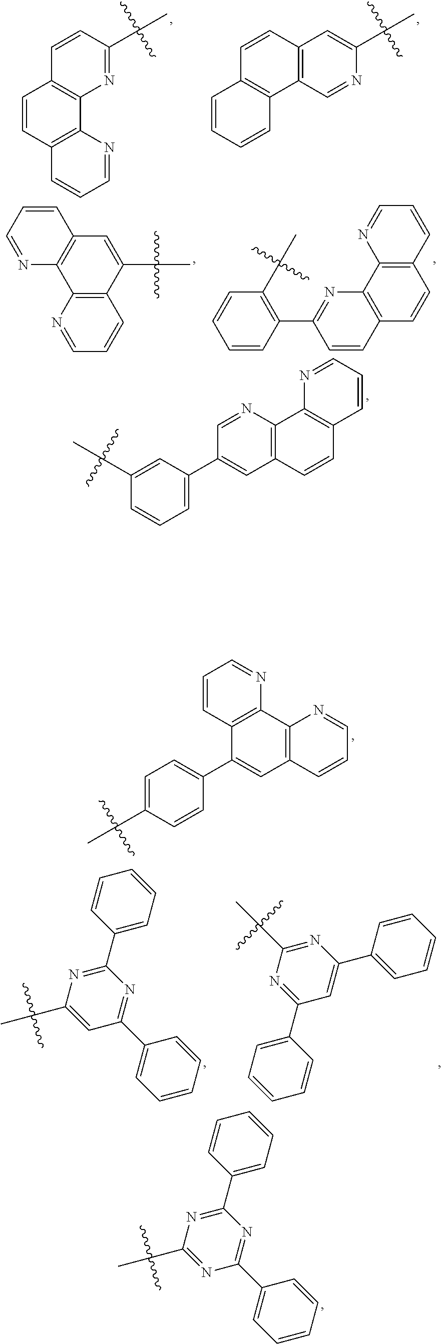

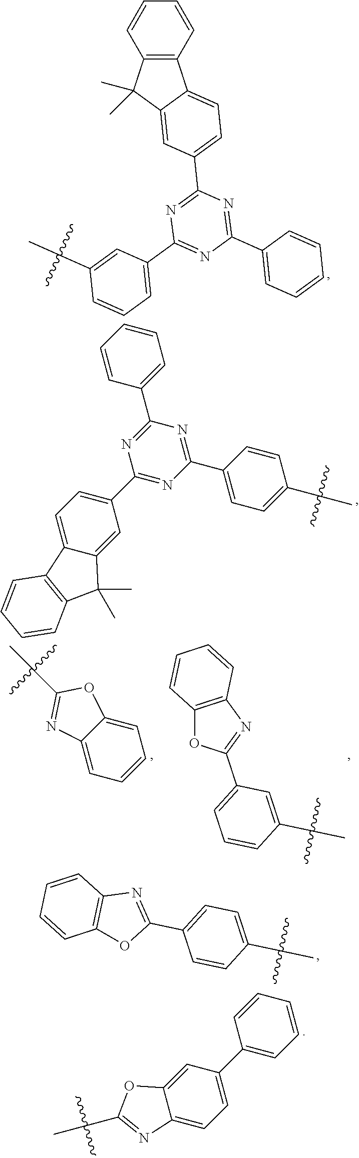

16. The compound, or a stereoisomer or tautomer thereof according to claim 12, wherein the compound has the structure represented by Formula II-4, 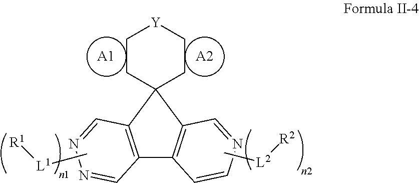
wherein:
Ring A1 and Ring A2 are each independently selected from benzene ring and naphthalene ring;
preferably, Ring A1 and Ring A2 are benzene rings;
Y is selected from —O—, —S—, —CR^{a}R^{b}—;
R^{a} and R^{b} are each independently selected from hydrogen and C₁-C₆ alkyl;
preferably, R^{a} and R^{b} are each independently selected from C₁-C₆ alkyl;
more preferably, R^{a} and R^{b} are methyl groups;
preferably, Y is selected from —O—, —S—, 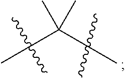 [SMILES: *C(*)(C)C]
more preferably, Y is selected from —O—, —S—;
n1 and n2 are each independently selected from 0 and 1, and n1 and n2 are not 0 at the same time;
preferably, 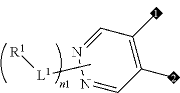
is selected from 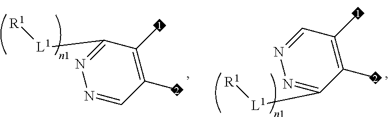
wherein Carbon atom No. 1 is bonded to the central spiro carbon atom, and Carbon atom No. 2 is bonded to a carbon atom of the aromatic ring on the right;
preferably, 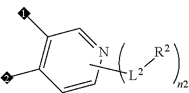
is selected from the group consisting of 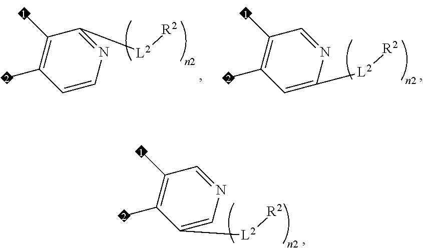
wherein, Carbon atom No. 1 is bonded to the central spiro carbon atom, and Carbon atom No. 2 is bonded to a carbon atom of the aromatic ring on the left;
L¹ and L² are each independently selected from the group consisting of a direct bond, phenylene, and naphthylene, and the phenylene and naphthylene are each independently optionally substituted by one R^{L};
preferably, L¹ and L² are each independently selected from the group consisting of a direct bond and phenylene, and the phenylene is optionally substituted by one R^{L};
more preferably, L¹ and L² are each independently selected from the group consisting of a direct bond, 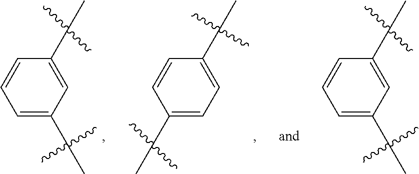 [SMILES: *c1cccc(*)c1 ; *c1ccc(*)cc1 ; *c1cccc(*)c1]
is optionally substituted by one R^{L};
R^{L} is selected from benzoxazolyl and benzothiazolyl;
preferably, R^{L} is benzoxazolyl;
more preferably, R^{L} is 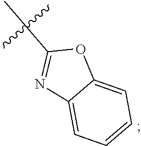 [SMILES: *c1nc2ccccc2o1]
most preferably, L¹ and L² are each independently selected from the group consisting of a direct bond, 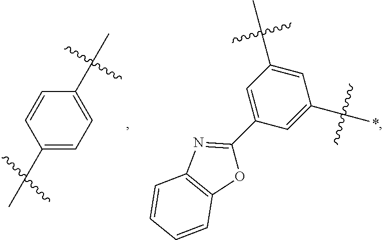
where the bond with marker * is bonded to the parent ring, and the bond without marker * is bonded to R¹ or R²;
R¹ and R² are each independently selected from the group consisting of diazaphenanthrolinyl, pyridinyl, triazinyl, benzoxazolyl, and benzothiazolyl, and the diazaphenanthrolinyl, pyridinyl, triazinyl, benzoxazolyl and benzothiazolyl are each independently optionally substituted by one or two (preferably two) R^{R};
preferably, R¹ and R² are each independently selected from the group consisting of diazaphenanthrolinyl, pyridinyl, triazinyl, and benzoxazolyl, and the pyridinyl, triazinyl, and benzoxazolyl are each independently optionally substituted by one or two (preferably two) R^{R};
more preferably, R¹ and R² are each independently selected from o-diazaphenanthrolinyl, pyridinyl, 1,3,5-triazinyl, and benzoxazolyl, and the pyridinyl, 1,3,5-triazinyl, and benzoxazolyl are each independently optionally substituted by one or two (preferably two) R^{R};
further preferably, R¹ and R² are each independently selected from the group consisting of 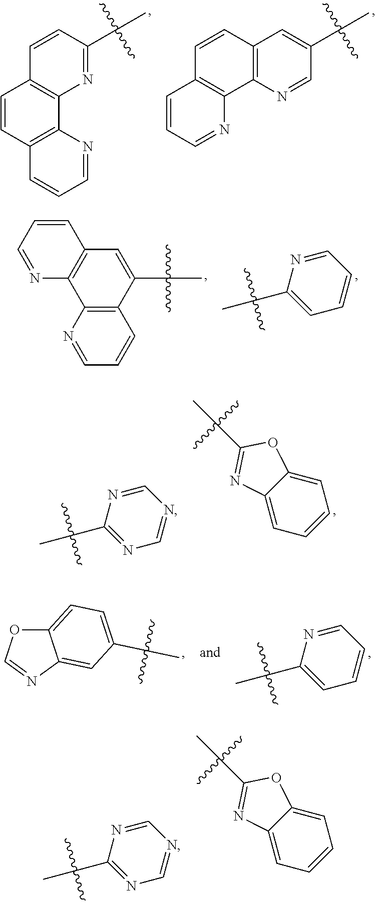
are each independently optionally substituted by one or two R^{R};
R^{R} is selected from phenyl and biphenyl, and the phenyl and biphenyl are independently optionally substituted by two C₁-C₆ alkyl groups;
preferably, R^{R} is selected from phenyl and biphenyl;
more preferably, R^{R} is selected from 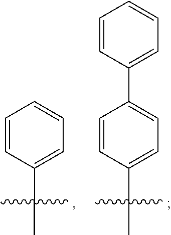
most preferably, R¹ and R² are each independently selected from the group consisting of: 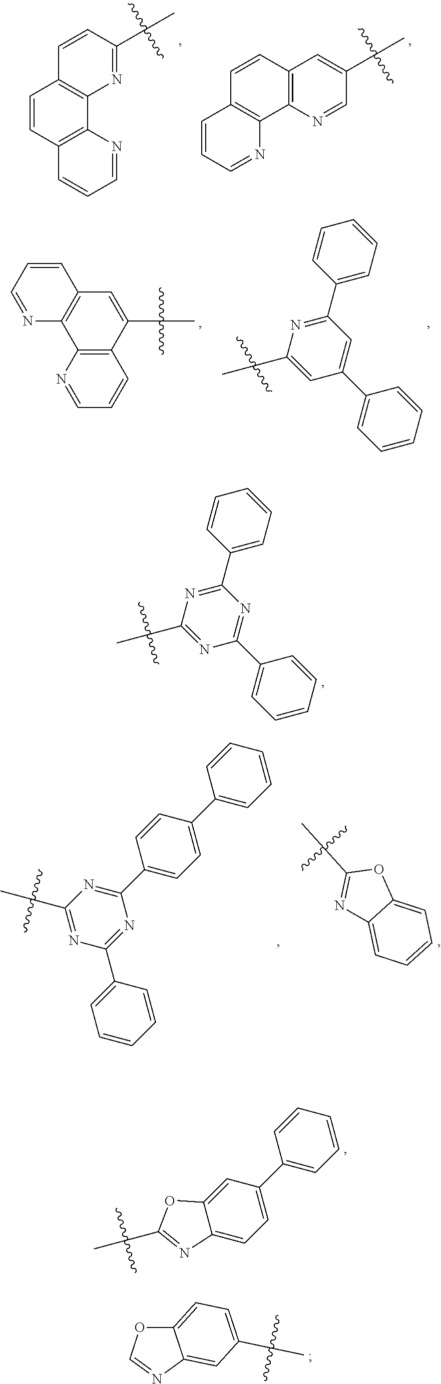
most preferably, R¹-L¹, R²-L²- are each independently selected from the group consisting of:

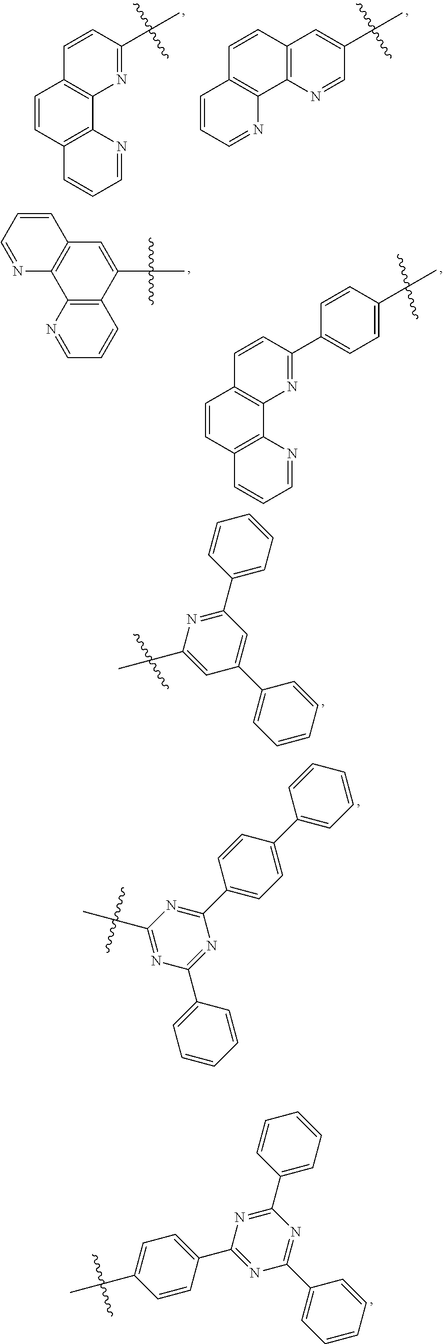

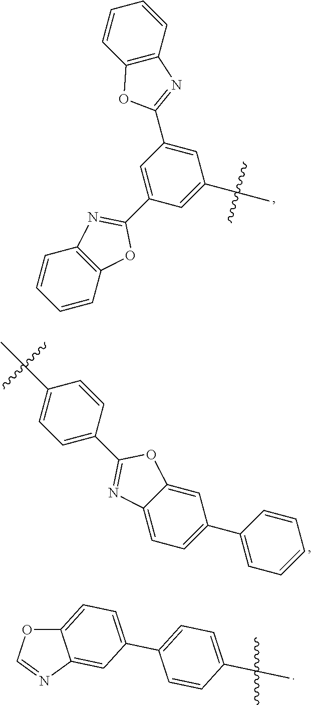 [SMILES: *c1cc(-c2nc3ccccc3o2)cc(-c2nc3ccccc3o2)c1 ; *c1ccc(-c2nc3ccc(-c4ccccc4)cc3o2)cc1 ; *c1ccc(-c2ccc3ocnc3c2)cc1]

17. The compound, or a stereoisomer or tautomer thereof according to claim 12, wherein the compound has the structure represented by Formula II-5, 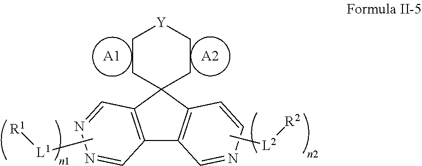
wherein:
Ring A1 and Ring A2 are each independently selected from benzene ring and naphthalene ring;
preferably, Ring A1 and Ring A2 are benzene rings;
Y is selected from —O—, —S—, —CR^{a}R^{b}—;
R^{a} and R^{b} are each independently selected from hydrogen and C₁-C₆ alkyl;
preferably, R^{a} and R^{b} are each independently selected from C₁-C₆ alkyl;
more preferably, R^{a} and R^{b} are methyl groups;
preferably, Y is selected from —O—, —S—, 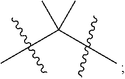 [SMILES: *C(*)(C)C]
more preferably, Y is selected from —O—, —S—;
n1 and n2 are each independently selected from 0 and 1, and n1 and n2 are not 0 at the same time;
preferably, 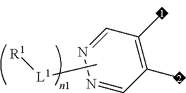
is selected from 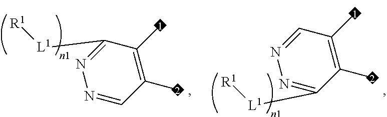
wherein Carbon atom No. 1 is bonded to the central spiro carbon atom, and Carbon atom No. 2 is bonded to a carbon atom of the aromatic ring on the right;
preferably, 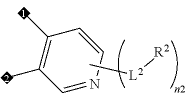
is selected from the group consisting of 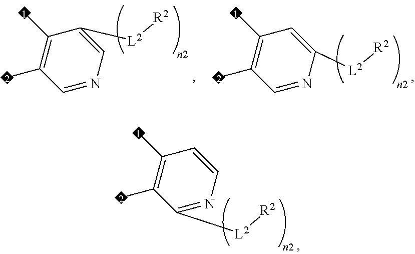
wherein, Carbon atom No. 1 is bonded to the central spiro carbon atom, and Carbon atom No. 2 is bonded to a carbon atom of the aromatic ring on the left;
L¹ and L² are each independently selected from the group consisting of a direct bond, phenylene, and naphthylene, and the phenylene and naphthylene are each independently optionally substituted by one R^{L};
preferably, L¹ and L² are each independently selected from the group consisting of a direct bond and phenylene, and the phenylene is optionally substituted by one R¹;
more preferably, L¹ and L² are each independently selected from the group consisting of a direct bond, 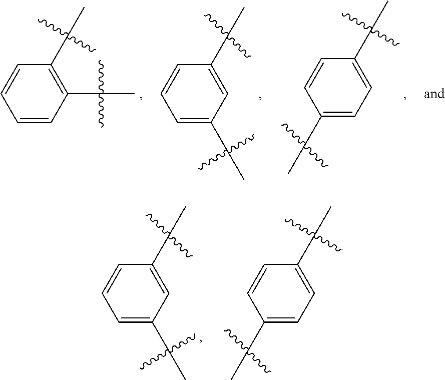 [SMILES: *c1ccccc1* ; *c1cccc(*)c1 ; *c1ccc(*)cc1 ; *c1cccc(*)c1 ; *c1ccc(*)cc1]
are each independently optionally substituted by one R^{L};
R^{L} is selected from benzoxazolyl and benzothiazolyl;
preferably, R^{L} is benzoxazolyl;
more preferably, R^{L} is selected from 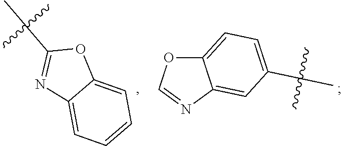
most preferably, L¹ and L² are each independently selected from the group consisting of:
a direct bond, 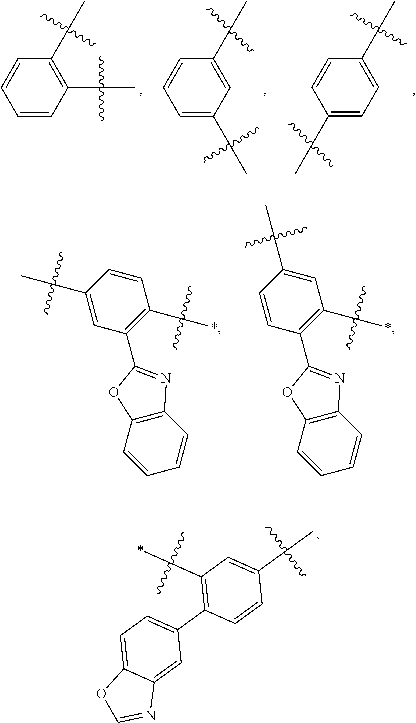
wherein, the bond with marker * is bonded to the parent ring, and the bond without marker * is bonded to R¹ or R²;
R¹ and R² are each independently selected from the group consisting of diazaphenanthrolinyl, pyrimidinyl, triazinyl, benzoxazolyl, benzothiazolyl, and the diazaphenanthrolinyl, pyrimidinyl, triazinyl, benzoxazolyl and benzothiazolyl are each independently optionally substituted by one or two (preferably two) R^{R};
preferably, R¹ and R² are each independently selected from the group consisting of diazaphenanthrolinyl, pyrimidinyl, triazinyl, and benzoxazolyl, and the pyrimidinyl, triazinyl, and benzoxazolyl are each independently optionally substituted by one or two (preferably two) R^{R};
more preferably, R¹ and R² are each independently selected from o-diazaphenanthrolinyl, pyrimidinyl, 1,3,5-triazinyl, and benzoxazolyl, and the pyrimidinyl, 1,3,5-triazinyl, and benzoxazolyl are each independently optionally substituted by one or two (preferably two) R^{R};
further preferably, R¹ and R² are each independently selected from the group consisting of 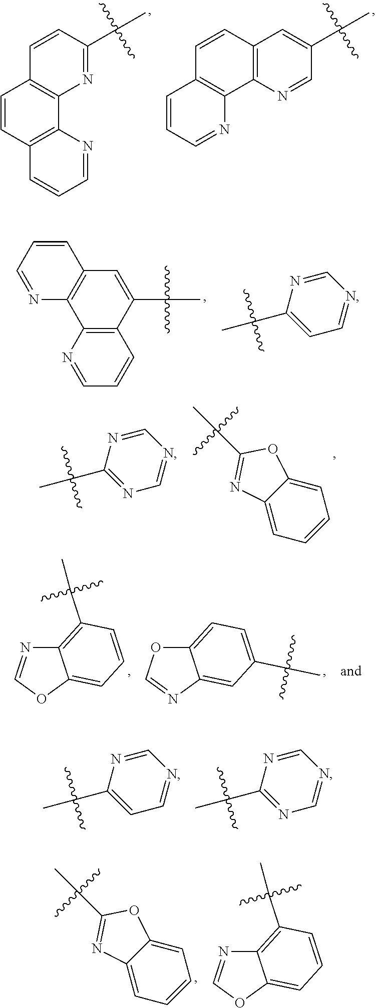
are each independently optionally substituted by one or two R^{R};
R^{R} is selected from the group consisting of phenyl, biphenyl, and naphthyl, and the phenyl, biphenyl, and naphthyl are each independently optionally substituted by two C₁-C₆ alkyl groups;
preferably, R^{R} is selected from the group consisting of phenyl, biphenyl, and naphthyl;
more preferably, R^{R} is selected from the group consisting of 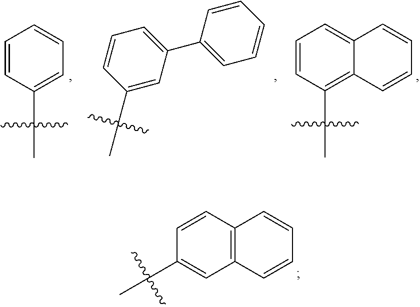
most preferably, R¹ and R² are each independently selected from the group consisting of: 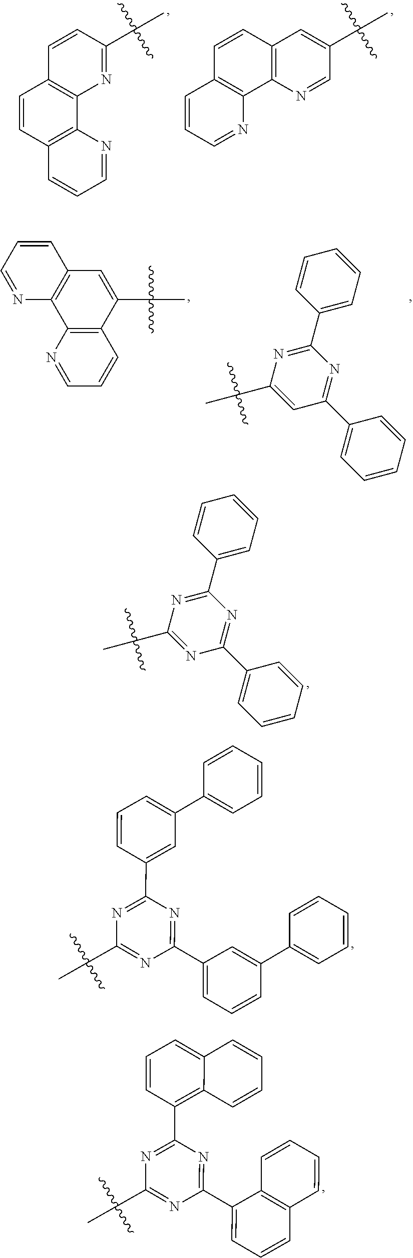 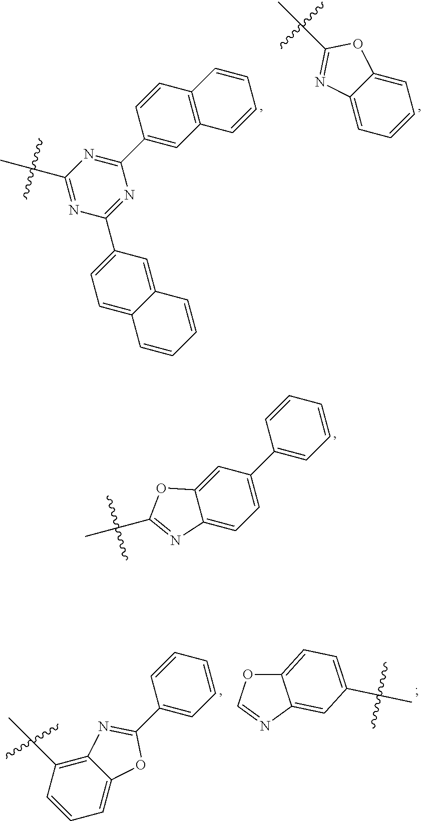
most preferably, R¹-L¹-, R²-L²- are each independently selected from the group consisting of:

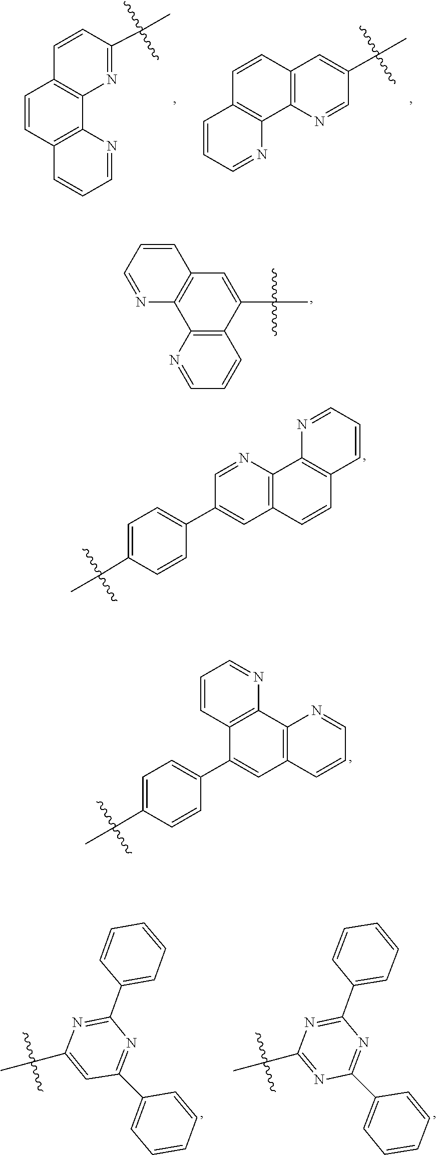

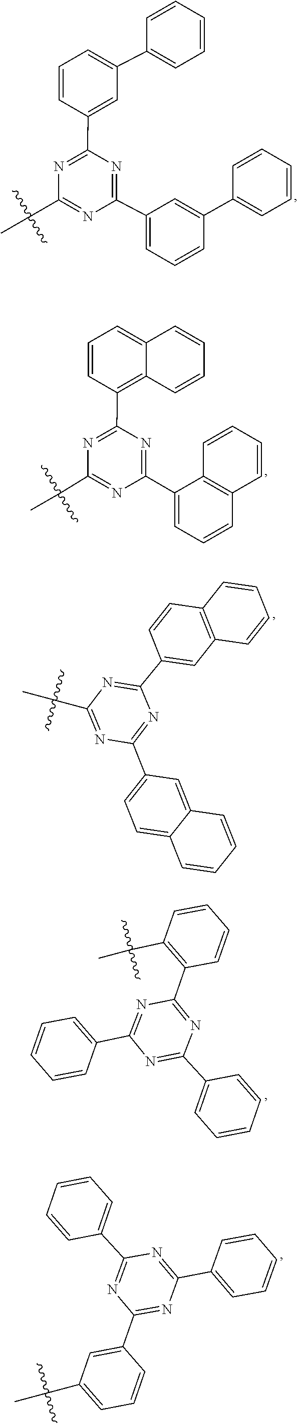

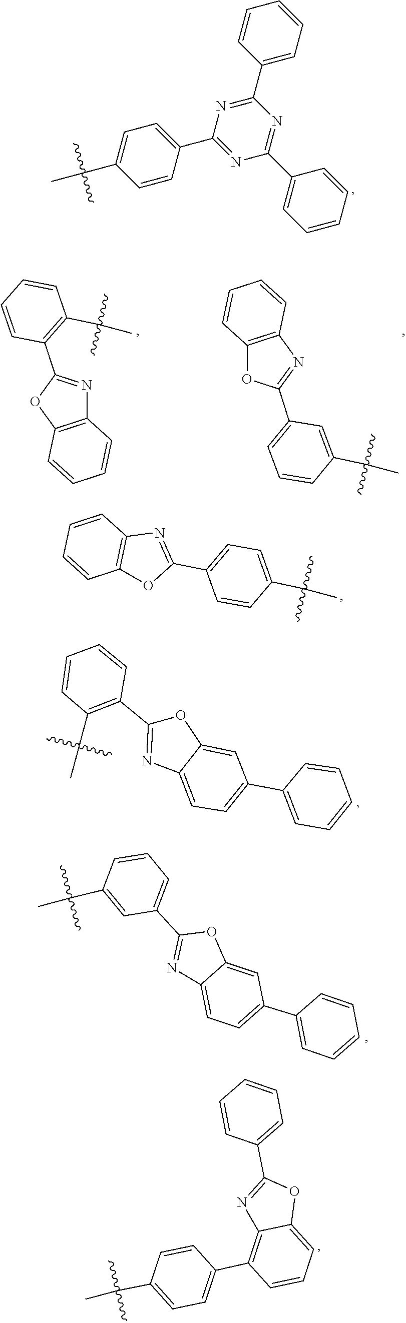

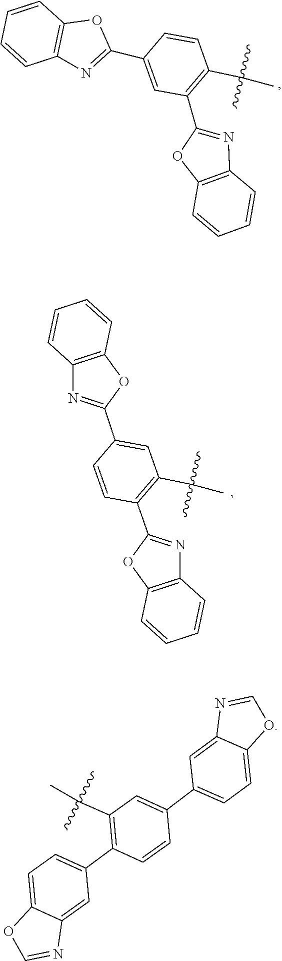 [SMILES: *c1ccc(-c2nc3ccccc3o2)cc1-c1nc2ccccc2o1 ; *c1cc(-c2nc3ccccc3o2)ccc1-c1nc2ccccc2o1 ; *c1cc(-c2ccc3ocnc3c2)ccc1-c1ccc2ocnc2c1]

18. The compound, or a stereoisomer or tautomer thereof according to claim 1, wherein the compound has the structure represented by Formula Oc-1, Formula Oc-2, or Formula Oc-3, 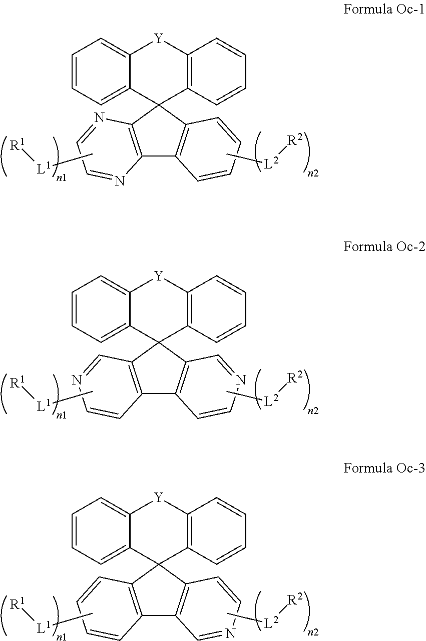
wherein:
Y is selected from O and S;
n1 and n2 are each independently selected from 0 and 1, and n1 and n2 are not 0 at the same time;
L¹ and L² are each independently selected from the group consisting of a direct bond, phenylene, naphthylene, and biphenylene, and the phenylene, naphthylene, and biphenylene are each independently optionally substituted by one R^{L};
preferably, L¹ and L² are each independently selected from the group consisting of a direct bond, phenylene, naphthylene, and biphenylene, and the phenylene is optionally substituted by one R^{L};
more preferably, L¹ and L² are each independently selected from the group consisting of a direct bond, 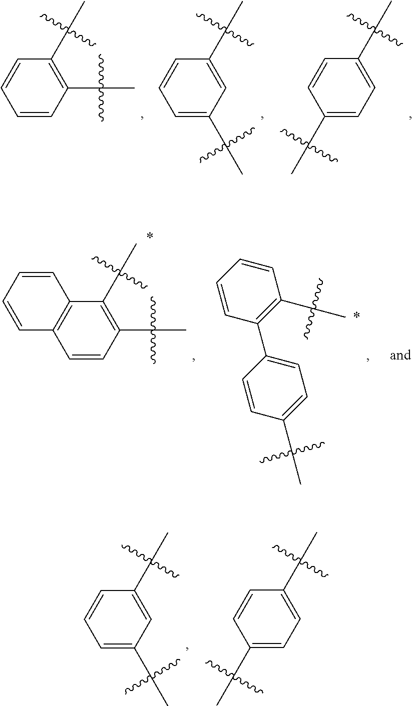
are each independently optionally substituted by one R^{L}, wherein the bond with marker * is bonded to the parent ring, and the bond without marker * is bonded to R¹ or R²;
R^{L} is selected from benzoxazolyl and benzothiazolyl;
preferably, R^{L} is benzoxazolyl;
more preferably, R^{L} is selected from 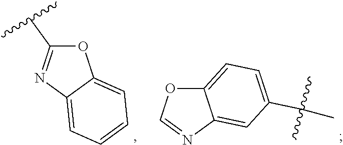
most preferably, L¹ and L² are each independently selected from the group consisting of:
a direct bond, 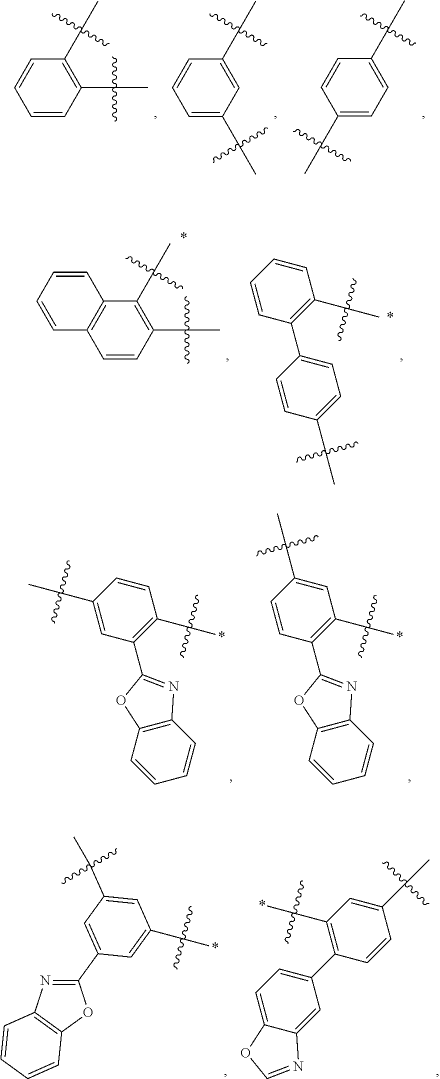
wherein, the bond with marker * is bonded to the parent ring, and the bond without marker * is bonded to R¹ or R²;
R¹ and R² are each independently selected from the group consisting of diazaphenanthrolinyl, pyridinyl, pyrimidinyl, triazinyl, benzoxazolyl, benzothiazolyl, and the diazaphenanthrolinyl, pyridinyl, pyrimidinyl, triazinyl, benzoxazolyl and benzothiazolyl are each independently optionally substituted by one or two R^{R};
preferably, R¹ and R² are each independently selected from the group consisting of diazaphenanthrolinyl, pyridinyl, pyrimidinyl, triazinyl, benzoxazolyl, benzothiazolyl, and the pyridinyl, pyrimidinyl, triazinyl, benzoxazolyl are each independently optionally substituted by one or two R^{R};
more preferably, R¹ and R² are each independently selected from o-diazaphenanthrolinyl, pyridinyl, pyrimidinyl, 1,3,5-triazinyl, benzoxazolyl, and the pyridinyl, pyrimidinyl, 1,3,5-triazinyl, benzoxazolyl are each independently optionally substituted by one or two R^{R};
further preferably, R¹ and R² are each independently selected from the group consisting of 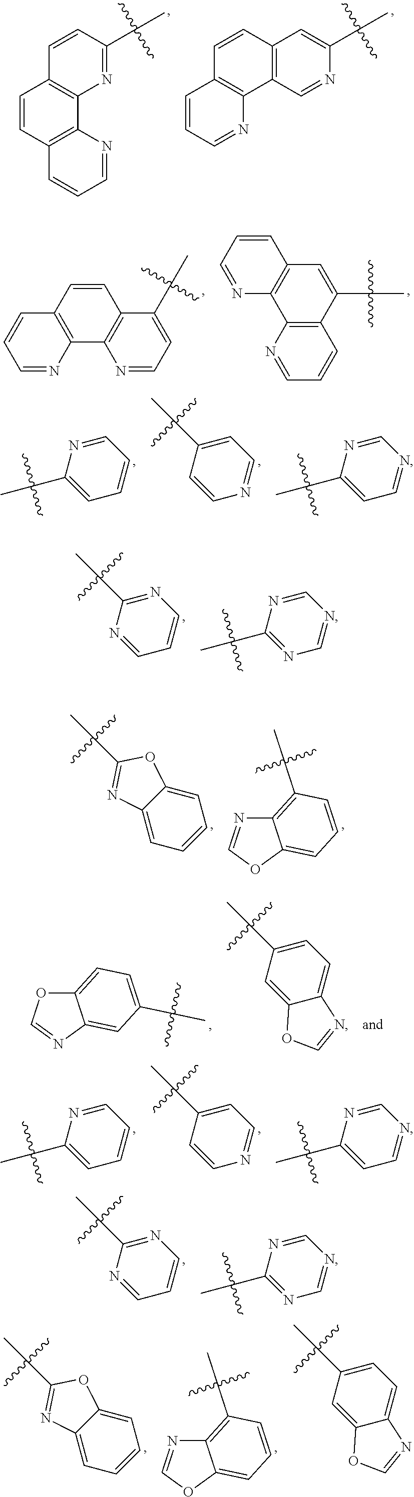
are each independently optionally substituted by one or two R^{R};
R^{R} is selected from the group consisting of phenyl, biphenyl, naphthyl, and fluorenyl, and the phenyl, biphenyl, naphthyl, and fluorenyl are each independently optionally substituted by two C₁-C₆ alkyl groups;
preferably, R^{R} is selected from the group consisting of phenyl, biphenyl, naphthyl, and fluorenyl, and the fluorenyl is optionally substituted by two C₁-C₆ alkyl groups;
more preferably, R^{R} is selected from the group consisting of phenyl, biphenyl, naphthyl, and fluorenyl, and the fluorenyl is optionally substituted by two methyl groups;
most preferably, R^{R} is selected from the group consisting of 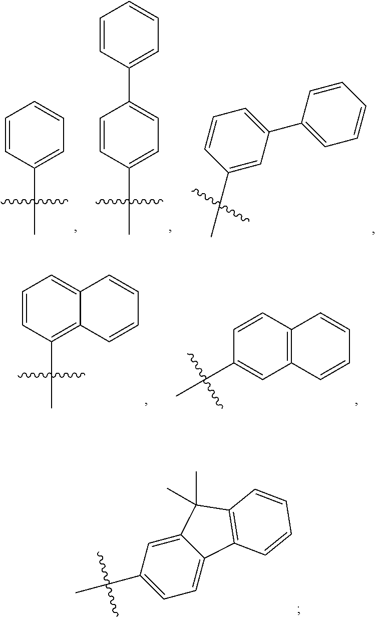
most preferably, R¹ and R² are each independently selected from the group consisting of: 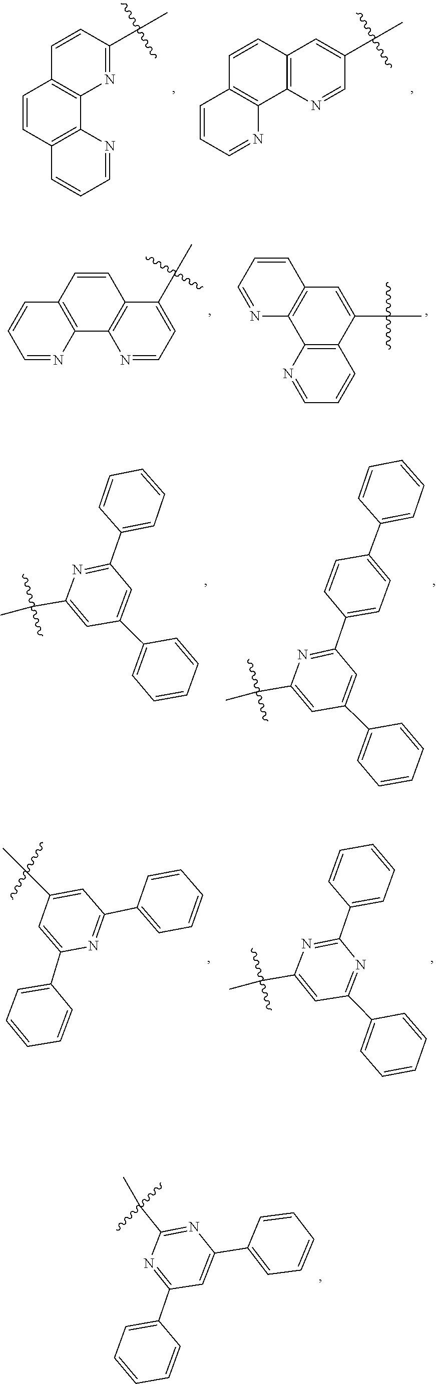 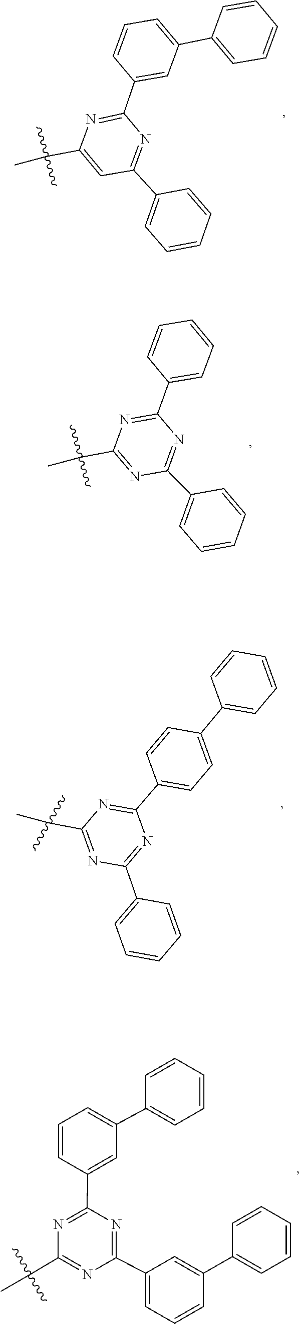 [SMILES: *c1cc(-c2ccccc2)nc(-c2cccc(-c3ccccc3)c2)n1 ; *c1nc(-c2ccccc2)nc(-c2ccccc2)n1 ; *c1nc(-c2ccccc2)nc(-c2ccc(-c3ccccc3)cc2)n1 ; *c1nc(-c2cccc(-c3ccccc3)c2)nc(-c2cccc(-c3ccccc3)c2)n1] 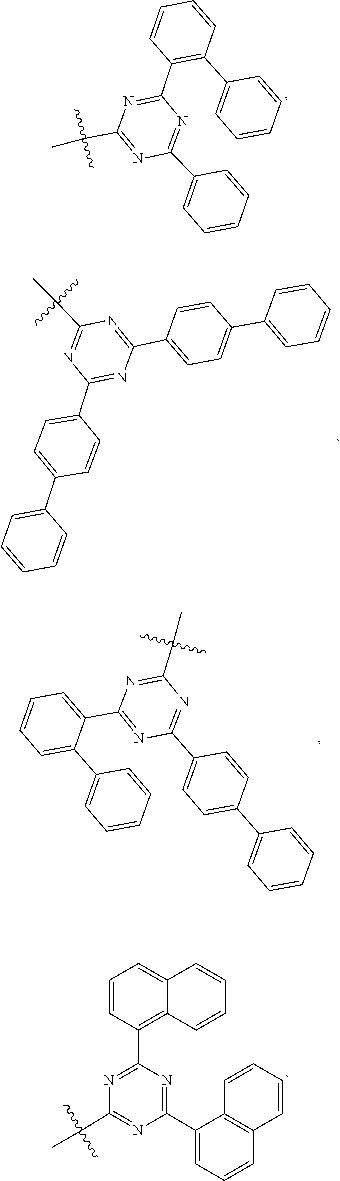 [SMILES: *c1nc(-c2ccccc2)nc(-c2ccccc2-c2ccccc2)n1 ; *c1nc(-c2ccc(-c3ccccc3)cc2)nc(-c2ccc(-c3ccccc3)cc2)n1 ; *c1nc(-c2ccc(-c3ccccc3)cc2)nc(-c2ccccc2-c2ccccc2)n1 ; *c1nc(-c2cccc3ccccc23)nc(-c2cccc3ccccc23)n1] 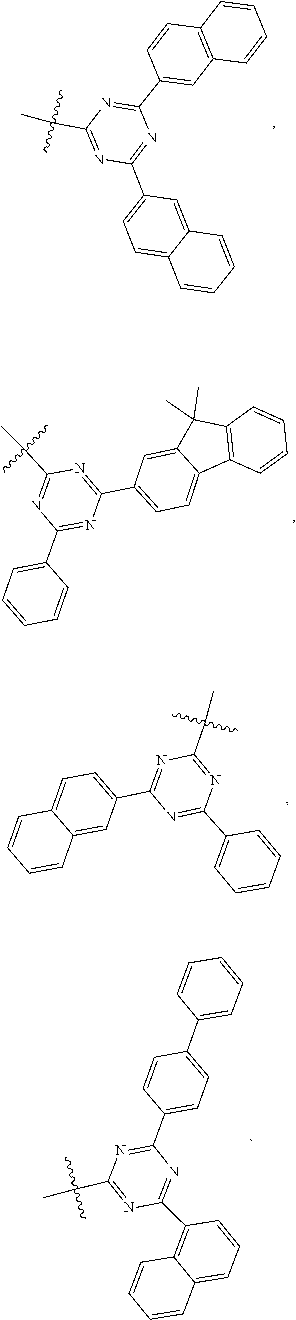 [SMILES: *c1nc(-c2ccc3ccccc3c2)nc(-c2ccc3ccccc3c2)n1 ; *c1nc(-c2ccccc2)nc(-c2ccc3c(c2)C(C)(C)c2ccccc2-3)n1 ; *c1nc(-c2ccccc2)nc(-c2ccc3ccccc3c2)n1 ; *c1nc(-c2ccc(-c3ccccc3)cc2)nc(-c2cccc3ccccc23)n1] 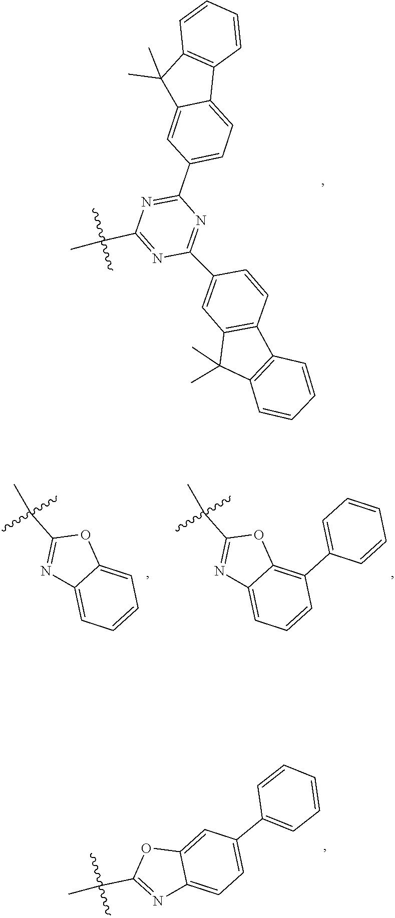 [SMILES: *c1nc(-c2ccc3c(c2)C(C)(C)c2ccccc2-3)nc(-c2ccc3c(c2)C(C)(C)c2ccccc2-3)n1 ; *c1nc2ccccc2o1 ; *c1nc2cccc(-c3ccccc3)c2o1 ; *c1nc2ccc(-c3ccccc3)cc2o1] 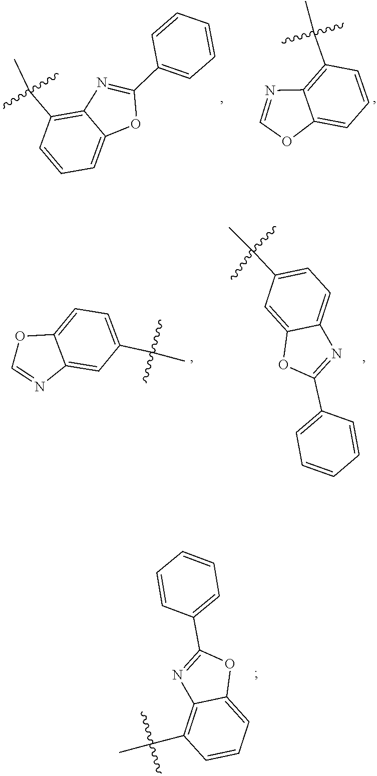 [SMILES: *c1cccc2oc(-c3ccccc3)nc12 ; *c1cccc2ocnc12 ; *c1ccc2ocnc2c1 ; *c1ccc2nc(-c3ccccc3)oc2c1 ; *c1cccc2oc(-c3ccccc3)nc12]
most preferably, R¹-L¹-, R²-L²- are each independently selected from the group consisting of:

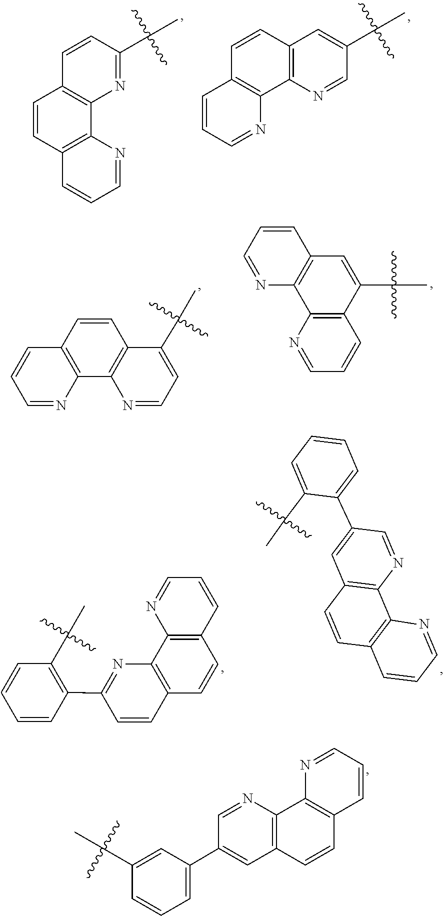

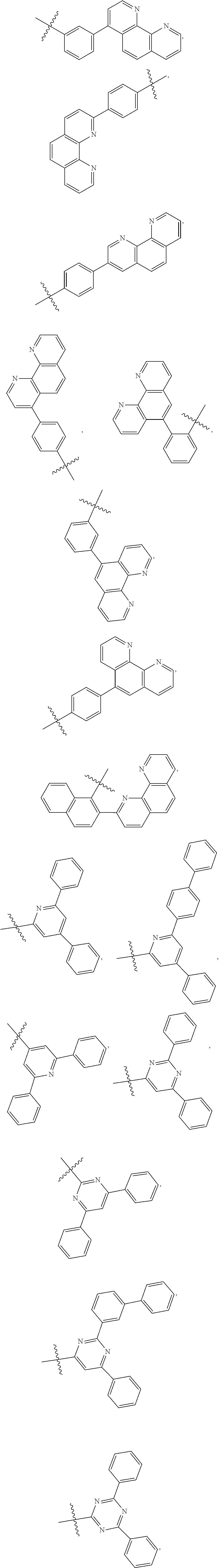

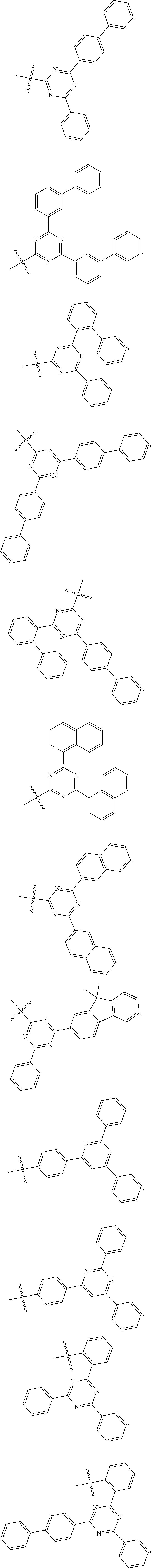

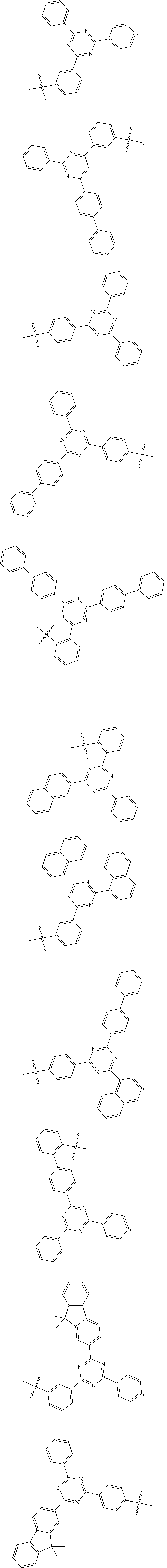

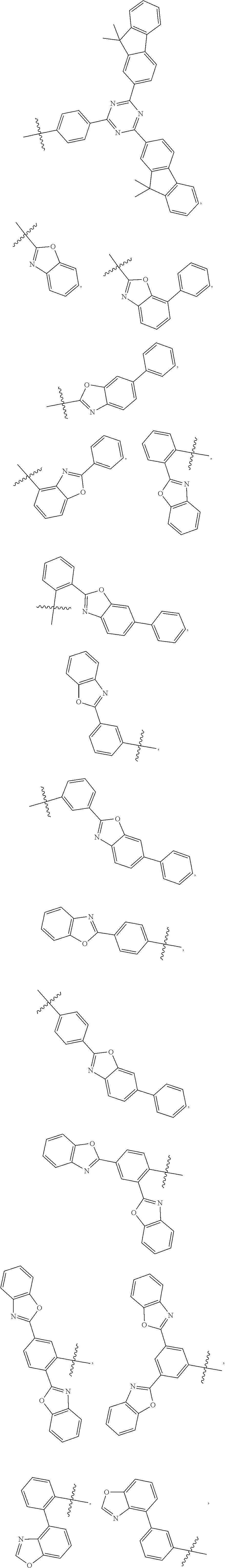

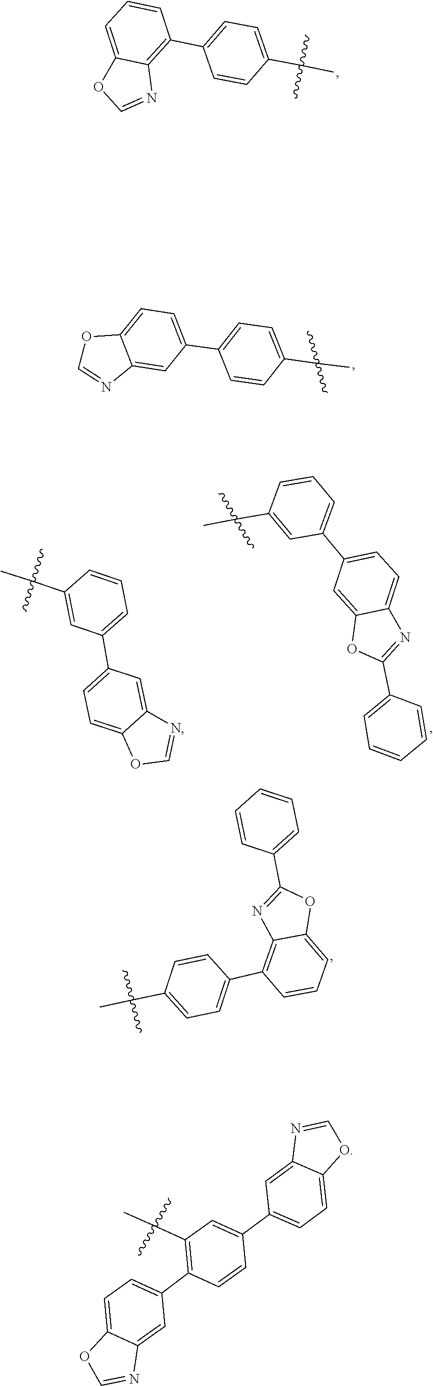 [SMILES: *c1ccc(-c2cccc3ocnc23)cc1 ; *c1ccc(-c2ccc3ocnc3c2)cc1 ; *c1cccc(-c2ccc3ocnc3c2)c1 ; *c1cccc(-c2ccc3nc(-c4ccccc4)oc3c2)c1 ; *c1ccc(-c2cccc3oc(-c4ccccc4)nc23)cc1 ; *c1cc(-c2ccc3ocnc3c2)ccc1-c1ccc2ocnc2c1]

19. The compound, or a stereoisomer or tautomer thereof according to claim 1, wherein the compound is selected from the group consisting of:

| No. | Structural formula |
|---|---|
| 1 | 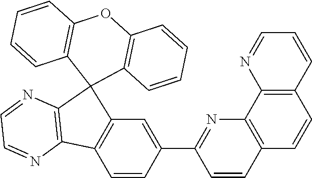 [SMILES: c1ccc2c(c1)Oc1ccccc1C21c2cc(-c3ccc4ccc5cccnc5c4n3)ccc2-c2nccnc21] |
| 2 | 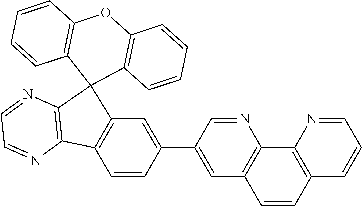 [SMILES: c1ccc2c(c1)Oc1ccccc1C21c2cc(-c3cnc4c(ccc5cccnc54)c3)ccc2-c2nccnc21] |
| 3 | 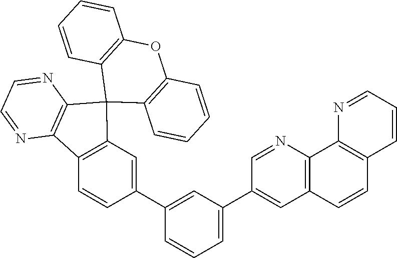 [SMILES: c1cc(-c2ccc3c(c2)C2(c4ccccc4Oc4ccccc42)c2nccnc2-3)cc(-c2cnc3c(ccc4cccnc43)c2)c1] |
| 4 | 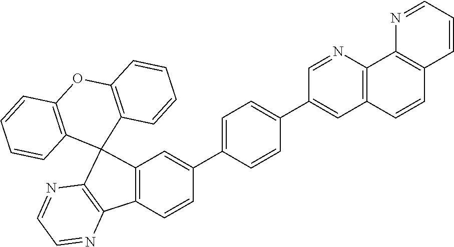 [SMILES: c1ccc2c(c1)Oc1ccccc1C21c2cc(-c3ccc(-c4cnc5c(ccc6cccnc65)c4)cc3)ccc2-c2nccnc21] |
| 5 | 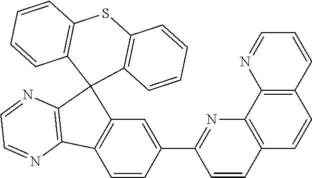 [SMILES: c1ccc2c(c1)Sc1ccccc1C21c2cc(-c3ccc4ccc5cccnc5c4n3)ccc2-c2nccnc21] |
| 6 | 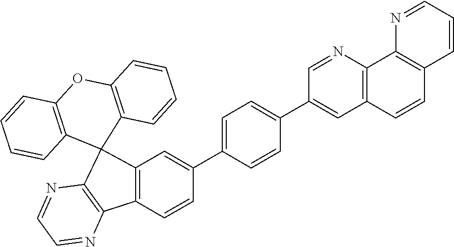 [SMILES: c1ccc2c(c1)Oc1ccccc1C21c2cc(-c3ccc(-c4cnc5c(ccc6cccnc65)c4)cc3)ccc2-c2nccnc21] |
| 7 | 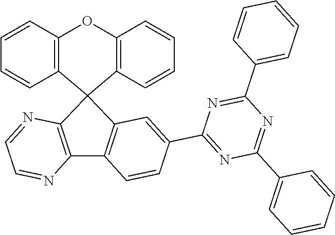 [SMILES: c1ccc(-c2nc(-c3ccccc3)nc(-c3ccc4c(c3)C3(c5ccccc5Oc5ccccc53)c3nccnc3-4)n2)cc1] |
| 8 | 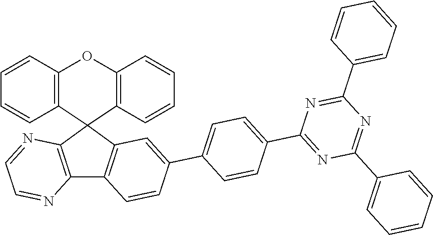 [SMILES: c1ccc(-c2nc(-c3ccccc3)nc(-c3ccc(-c4ccc5c(c4)C4(c6ccccc6Oc6ccccc64)c4nccnc4-5)cc3)n2)cc1] |
| 9 | 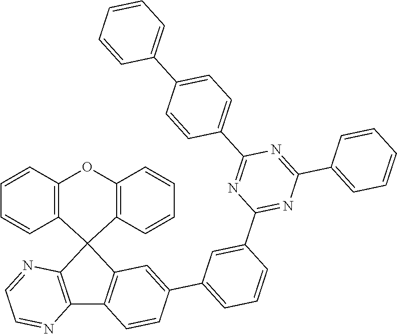 [SMILES: c1ccc(-c2ccc(-c3nc(-c4ccccc4)nc(-c4cccc(-c5ccc6c(c5)C5(c7ccccc7Oc7ccccc75)c5nccnc5-6)c4)n3)cc2)cc1] |
| 10 | 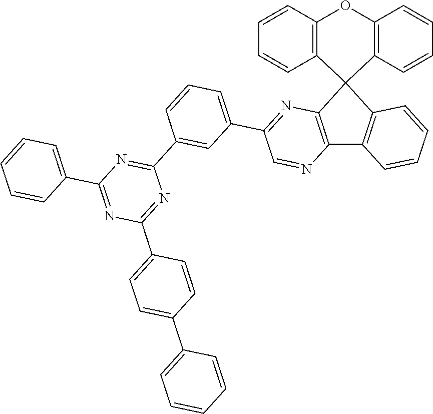 [SMILES: c1ccc(-c2ccc(-c3nc(-c4ccccc4)nc(-c4cccc(-c5cnc6c(n5)C5(c7ccccc7Oc7ccccc75)c5ccccc5-6)c4)n3)cc2)cc1] |
| 11 | 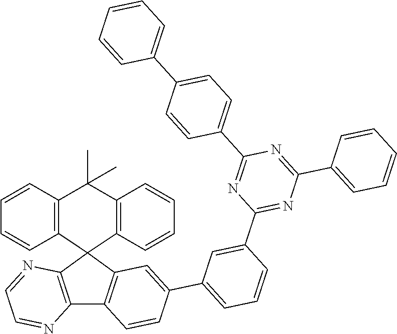 [SMILES: CC1(C)c2ccccc2C2(c3cc(-c4cccc(-c5nc(-c6ccccc6)nc(-c6ccc(-c7ccccc7)cc6)n5)c4)ccc3-c3nccnc32)c2ccccc21] |
| 12 | 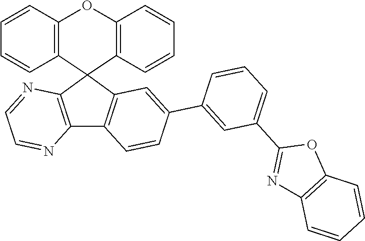 [SMILES: c1cc(-c2ccc3c(c2)C2(c4ccccc4Oc4ccccc42)c2nccnc2-3)cc(-c2nc3ccccc3o2)c1] |
| 13 | 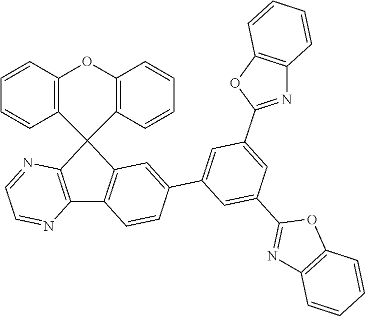 [SMILES: c1ccc2c(c1)Oc1ccccc1C21c2cc(-c3cc(-c4nc5ccccc5o4)cc(-c4nc5ccccc5o4)c3)ccc2-c2nccnc21] |
| 14 | 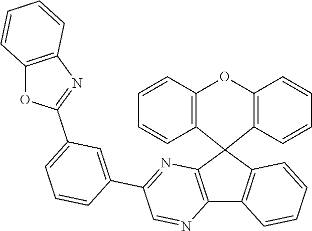 [SMILES: c1cc(-c2cnc3c(n2)C2(c4ccccc4Oc4ccccc42)c2ccccc2-3)cc(-c2nc3ccccc3o2)c1] |
| 15 | 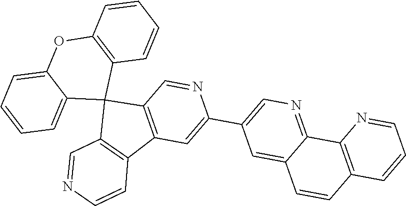 [SMILES: c1ccc2c(c1)Oc1ccccc1C21c2cnccc2-c2cc(-c3cnc4c(ccc5cccnc54)c3)ncc21] |
| 16 | 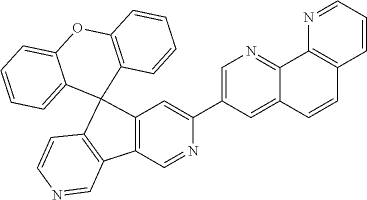 [SMILES: c1ccc2c(c1)Oc1ccccc1C21c2ccncc2-c2cnc(-c3cnc4c(ccc5cccnc54)c3)cc21] |
| 17 | 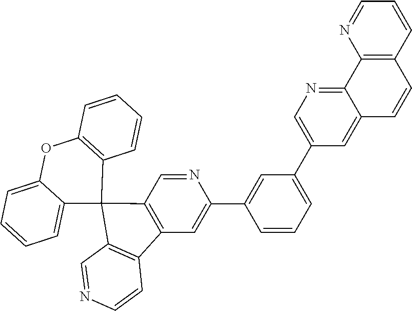 [SMILES: c1cc(-c2cnc3c(ccc4cccnc43)c2)cc(-c2cc3c(cn2)C2(c4ccccc4Oc4ccccc42)c2cnccc2-3)c1] |
| 18 | 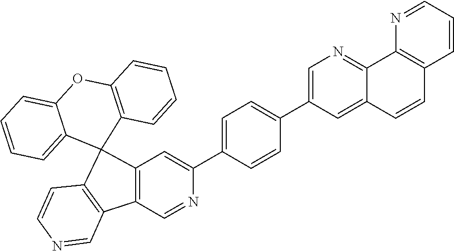 [SMILES: c1ccc2c(c1)Oc1ccccc1C21c2ccncc2-c2cnc(-c3ccc(-c4cnc5c(ccc6cccnc65)c4)cc3)cc21] |
| 19 | 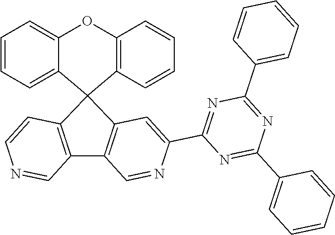 [SMILES: c1ccc(-c2nc(-c3ccccc3)nc(-c3cc4c(cn3)-c3cnccc3C43c4ccccc4Oc4ccccc43)n2)cc1] |
| 20 | 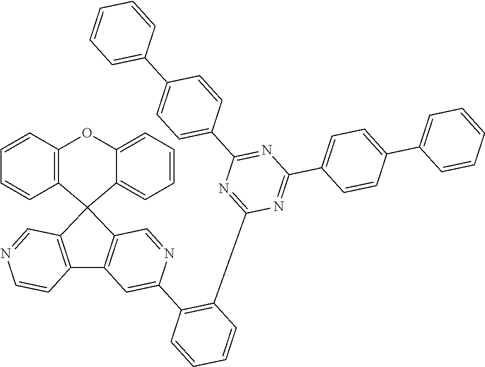 [SMILES: c1ccc(-c2ccc(-c3nc(-c4ccc(-c5ccccc5)cc4)nc(-c4ccccc4-c4cc5c(cn4)C4(c6ccccc6Oc6ccccc64)c4cnccc4-5)n3)cc2)cc1] |
| 21 | 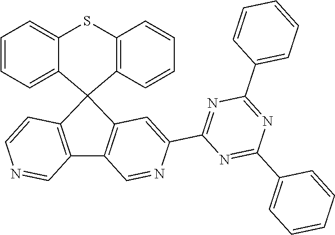 [SMILES: c1ccc(-c2nc(-c3ccccc3)nc(-c3cc4c(cn3)-c3cnccc3C43c4ccccc4Sc4ccccc43)n2)cc1] |
| 22 | 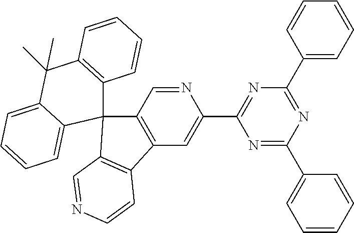 [SMILES: CC1(C)c2ccccc2C2(c3cnccc3-c3cc(-c4nc(-c5ccccc5)nc(-c5ccccc5)n4)ncc32)c2ccccc21] |
| 23 | 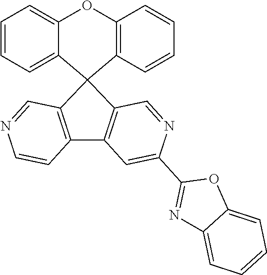 [SMILES: c1ccc2c(c1)Oc1ccccc1C21c2cnccc2-c2cc(-c3nc4ccccc4o3)ncc21] |
| 24 | 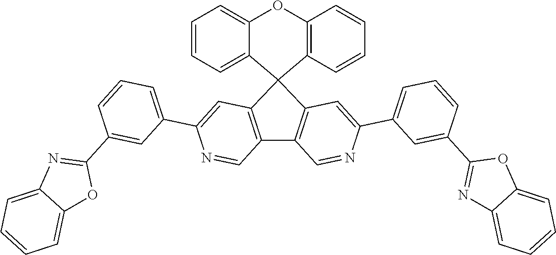 [SMILES: c1cc(-c2cc3c(cn2)-c2cnc(-c4cccc(-c5nc6ccccc6o5)c4)cc2C32c3ccccc3Oc3ccccc32)cc(-c2nc3ccccc3o2)c1] |
| 25 | 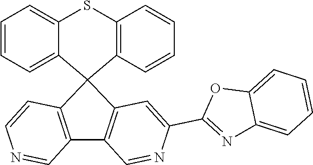 [SMILES: c1ccc2c(c1)Sc1ccccc1C21c2ccncc2-c2cnc(-c3nc4ccccc4o3)cc21] |
| 26 | 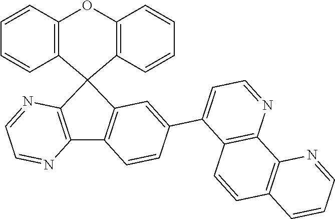 [SMILES: c1ccc2c(c1)Oc1ccccc1C21c2cc(-c3ccnc4c3ccc3cccnc34)ccc2-c2nccnc21] |
| 27 | 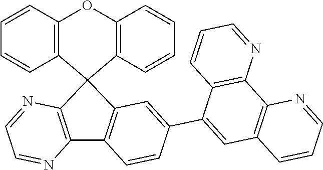 [SMILES: c1ccc2c(c1)Oc1ccccc1C21c2cc(-c3cc4cccnc4c4ncccc34)ccc2-c2nccnc21] |
| 28 | 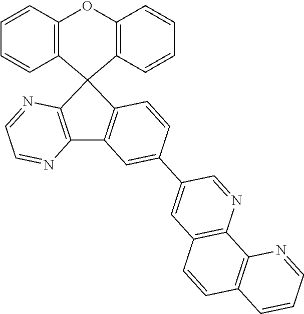 [SMILES: c1ccc2c(c1)Oc1ccccc1C21c2ccc(-c3cnc4c(ccc5cccnc54)c3)cc2-c2nccnc21] |
| 29 | 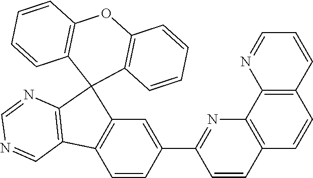 [SMILES: c1ccc2c(c1)Oc1ccccc1C21c2cc(-c3ccc4ccc5cccnc5c4n3)ccc2-c2cncnc21] |
| 30 | 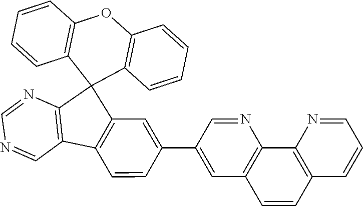 [SMILES: c1ccc2c(c1)Oc1ccccc1C21c2cc(-c3cnc4c(ccc5cccnc54)c3)ccc2-c2cncnc21] |
| 31 | 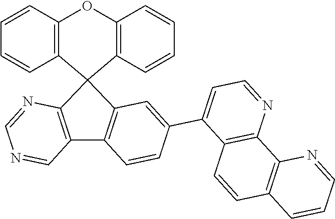 [SMILES: c1ccc2c(c1)Oc1ccccc1C21c2cc(-c3ccnc4c3ccc3cccnc34)ccc2-c2cncnc21] |
| 32 | 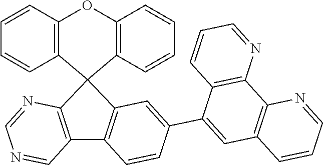 [SMILES: c1ccc2c(c1)Oc1ccccc1C21c2cc(-c3cc4cccnc4c4ncccc34)ccc2-c2cncnc21] |
| 33 | 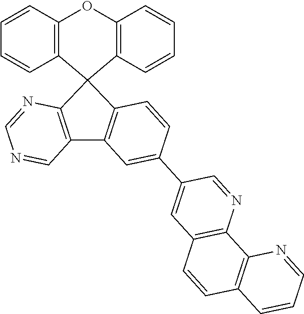 [SMILES: c1ccc2c(c1)Oc1ccccc1C21c2ccc(-c3cnc4c(ccc5cccnc54)c3)cc2-c2cncnc21] |
| 34 | 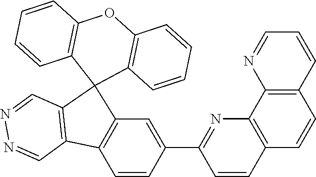 [SMILES: c1ccc2c(c1)Oc1ccccc1C21c2cnncc2-c2ccc(-c3ccc4ccc5cccnc5c4n3)cc21] |
| 35 | 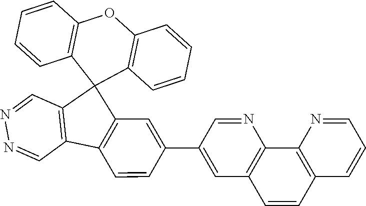 [SMILES: c1ccc2c(c1)Oc1ccccc1C21c2cnncc2-c2ccc(-c3cnc4c(ccc5cccnc54)c3)cc21] |
| 36 | 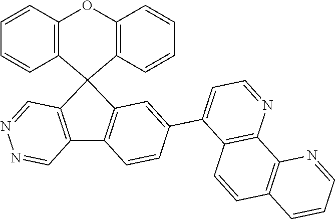 [SMILES: c1ccc2c(c1)Oc1ccccc1C21c2cnncc2-c2ccc(-c3ccnc4c3ccc3cccnc34)cc21] |
| 37 | 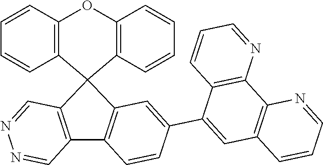 [SMILES: c1ccc2c(c1)Oc1ccccc1C21c2cnncc2-c2ccc(-c3cc4cccnc4c4ncccc34)cc21] |
| 38 | 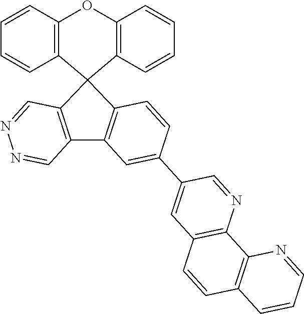 [SMILES: c1ccc2c(c1)Oc1ccccc1C21c2ccc(-c3cnc4c(ccc5cccnc54)c3)cc2-c2cnncc21] |
| 39 | 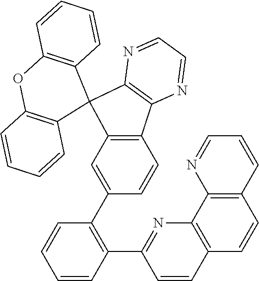 [SMILES: c1ccc2c(c1)Oc1ccccc1C21c2cc(-c3ccccc3-c3ccc4ccc5cccnc5c4n3)ccc2-c2nccnc21] |
| 40 | 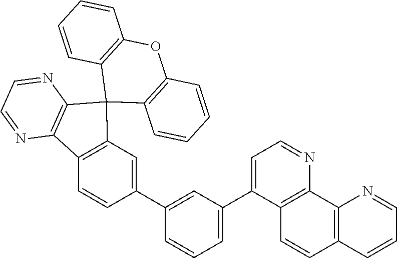 [SMILES: c1cc(-c2ccc3c(c2)C2(c4ccccc4Oc4ccccc42)c2nccnc2-3)cc(-c2ccnc3c2ccc2cccnc23)c1] |
| 41 | 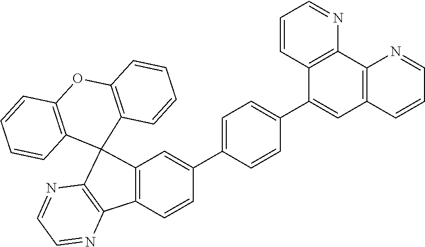 [SMILES: c1ccc2c(c1)Oc1ccccc1C21c2cc(-c3ccc(-c4cc5cccnc5c5ncccc45)cc3)ccc2-c2nccnc21] |
| 42 | 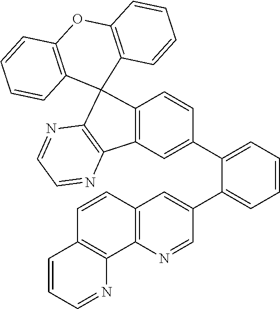 [SMILES: c1ccc2c(c1)Oc1ccccc1C21c2ccc(-c3ccccc3-c3cnc4c(ccc5cccnc54)c3)cc2-c2nccnc21] |
| 43 | 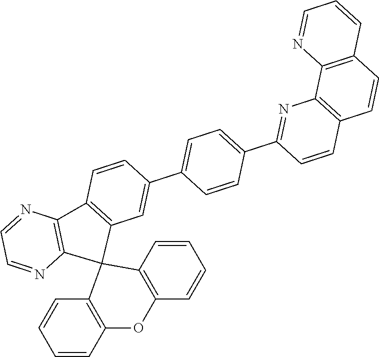 [SMILES: c1ccc2c(c1)Oc1ccccc1C21c2cc(-c3ccc(-c4ccc5ccc6cccnc6c5n4)cc3)ccc2-c2nccnc21] |
| 44 | 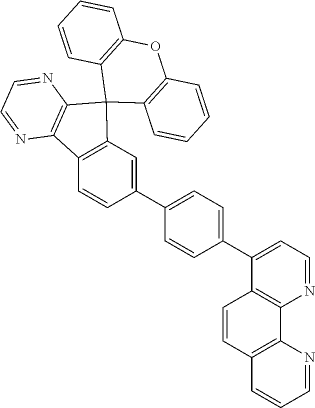 [SMILES: c1ccc2c(c1)Oc1ccccc1C21c2cc(-c3ccc(-c4ccnc5c4ccc4cccnc45)cc3)ccc2-c2nccnc21] |
| 45 | 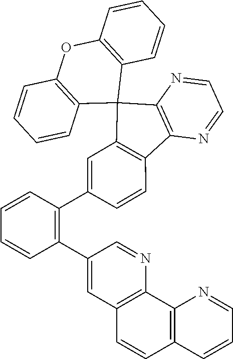 [SMILES: c1ccc2c(c1)Oc1ccccc1C21c2cc(-c3ccccc3-c3cnc4c(ccc5cccnc54)c3)ccc2-c2nccnc21] |
| 46 | 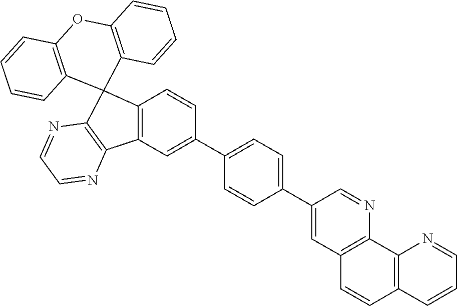 [SMILES: c1ccc2c(c1)Oc1ccccc1C21c2ccc(-c3ccc(-c4cnc5c(ccc6cccnc65)c4)cc3)cc2-c2nccnc21] |
| 47 | 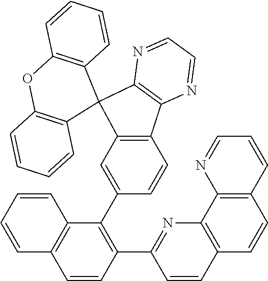 [SMILES: c1ccc2c(c1)Oc1ccccc1C21c2cc(-c3c(-c4ccc5ccc6cccnc6c5n4)ccc4ccccc34)ccc2-c2nccnc21] |
| 48 | 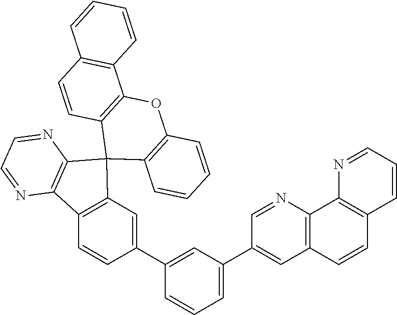 [SMILES: c1cc(-c2ccc3c(c2)C2(c4ccccc4Oc4c2ccc2ccccc42)c2nccnc2-3)cc(-c2cnc3c(ccc4cccnc43)c2)c1] |
| 49 | 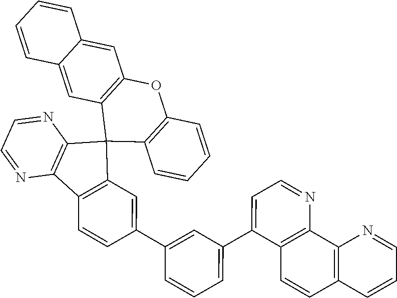 [SMILES: c1cc(-c2ccc3c(c2)C2(c4ccccc4Oc4cc5ccccc5cc42)c2nccnc2-3)cc(-c2ccnc3c2ccc2cccnc23)c1] |
| 50 | 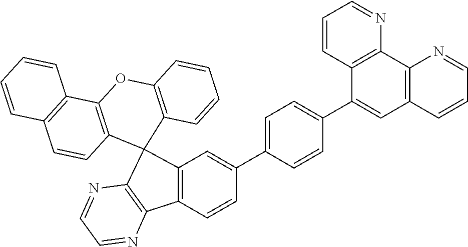 [SMILES: c1ccc2c(c1)Oc1c(ccc3ccccc13)C21c2cc(-c3ccc(-c4cc5cccnc5c5ncccc45)cc3)ccc2-c2nccnc21] |
| 51 | 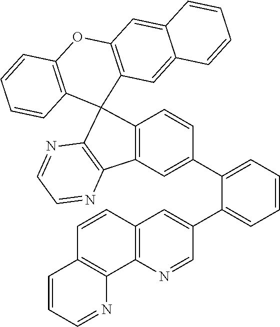 [SMILES: c1ccc2c(c1)Oc1cc3ccccc3cc1C21c2ccc(-c3ccccc3-c3cnc4c(ccc5cccnc54)c3)cc2-c2nccnc21] |
| 52 | 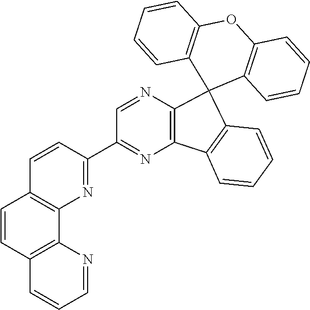 [SMILES: c1ccc2c(c1)Oc1ccccc1C21c2ccccc2-c2nc(-c3ccc4ccc5cccnc5c4n3)cnc21] |
| 53 | 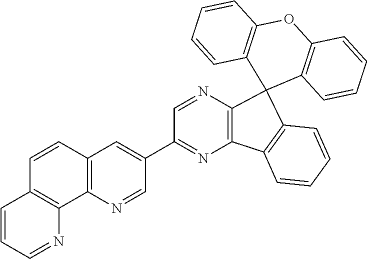 [SMILES: c1ccc2c(c1)Oc1ccccc1C21c2ccccc2-c2nc(-c3cnc4c(ccc5cccnc54)c3)cnc21] |
| 54 | 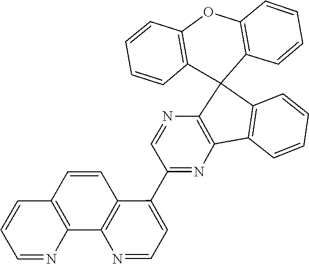 [SMILES: c1ccc2c(c1)Oc1ccccc1C21c2ccccc2-c2nc(-c3ccnc4c3ccc3cccnc34)cnc21] |
| 55 | 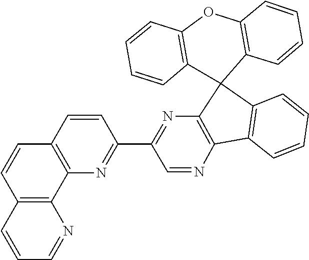 [SMILES: c1ccc2c(c1)Oc1ccccc1C21c2ccccc2-c2ncc(-c3ccc4ccc5cccnc5c4n3)nc21] |
| 56 | 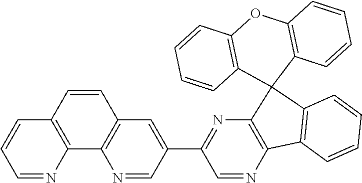 [SMILES: c1ccc2c(c1)Oc1ccccc1C21c2ccccc2-c2ncc(-c3cnc4c(ccc5cccnc54)c3)nc21] |
| 57 | 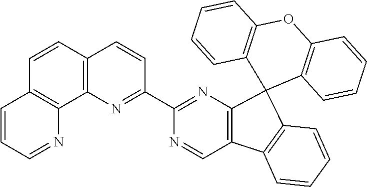 [SMILES: c1ccc2c(c1)Oc1ccccc1C21c2ccccc2-c2cnc(-c3ccc4ccc5cccnc5c4n3)nc21] |
| 58 | 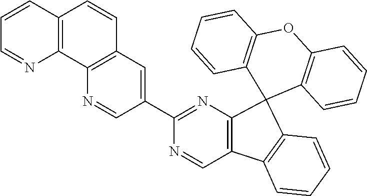 [SMILES: c1ccc2c(c1)Oc1ccccc1C21c2ccccc2-c2cnc(-c3cnc4c(ccc5cccnc54)c3)nc21] |
| 59 | 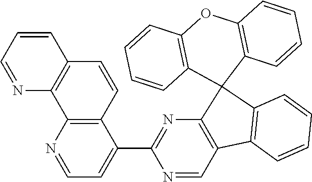 [SMILES: c1ccc2c(c1)Oc1ccccc1C21c2ccccc2-c2cnc(-c3ccnc4c3ccc3cccnc34)nc21] |
| 60 | 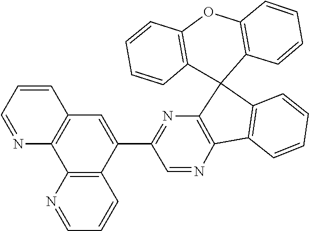 [SMILES: c1ccc2c(c1)Oc1ccccc1C21c2ccccc2-c2ncc(-c3cc4cccnc4c4ncccc34)nc21] |
| 61 | 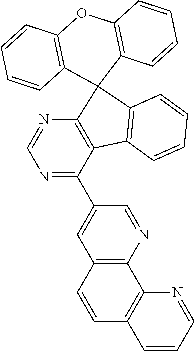 [SMILES: c1ccc2c(c1)Oc1ccccc1C21c2ccccc2-c2c(-c3cnc4c(ccc5cccnc54)c3)ncnc21] |
| 62 | 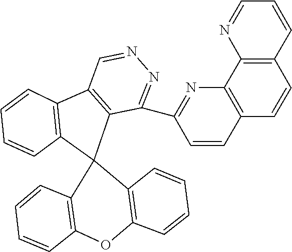 [SMILES: c1ccc2c(c1)Oc1ccccc1C21c2ccccc2-c2cnnc(-c3ccc4ccc5cccnc5c4n3)c21] |
| 63 | 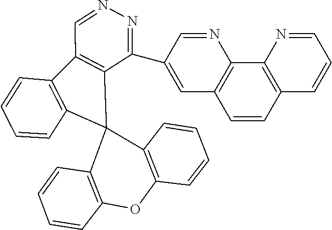 [SMILES: c1ccc2c(c1)Oc1ccccc1C21c2ccccc2-c2cnnc(-c3cnc4c(ccc5cccnc54)c3)c21] |
| 64 | 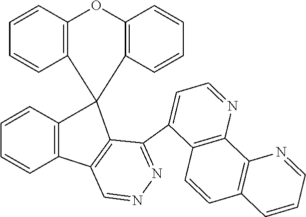 [SMILES: c1ccc2c(c1)Oc1ccccc1C21c2ccccc2-c2cnnc(-c3ccnc4c3ccc3cccnc34)c21] |
| 65 | 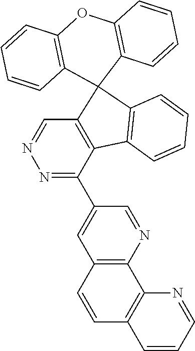 [SMILES: c1ccc2c(c1)Oc1ccccc1C21c2ccccc2-c2c1cnnc2-c1cnc2c(ccc3cccnc32)c1] |
| 66 | 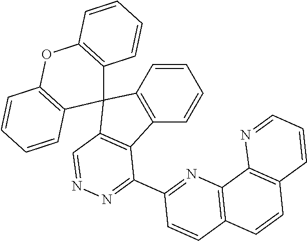 [SMILES: c1ccc2c(c1)Oc1ccccc1C21c2ccccc2-c2c1cnnc2-c1ccc2ccc3cccnc3c2n1] |
| 67 | 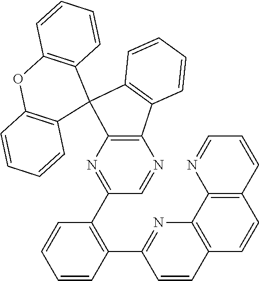 [SMILES: c1ccc2c(c1)Oc1ccccc1C21c2ccccc2-c2ncc(-c3ccccc3-c3ccc4ccc5cccnc5c4n3)nc21] |
| 68 | 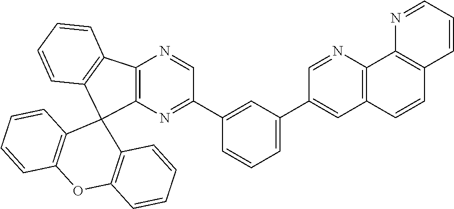 [SMILES: c1cc(-c2cnc3c(ccc4cccnc43)c2)cc(-c2cnc3c(n2)C2(c4ccccc4Oc4ccccc42)c2ccccc2-3)c1] |
| 69 | 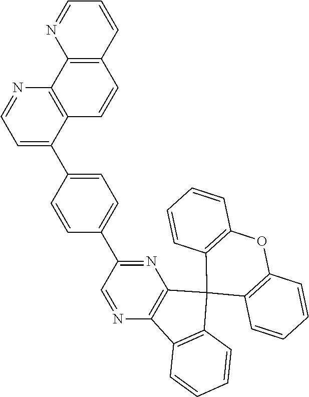 [SMILES: c1ccc2c(c1)Oc1ccccc1C21c2ccccc2-c2ncc(-c3ccc(-c4ccnc5c4ccc4cccnc45)cc3)nc21] |
| 70 | 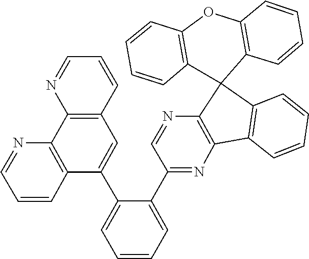 [SMILES: c1ccc2c(c1)Oc1ccccc1C21c2ccccc2-c2nc(-c3ccccc3-c3cc4cccnc4c4ncccc34)cnc21] |
| 71 | 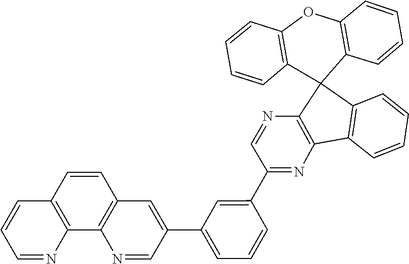 [SMILES: c1cc(-c2cnc3c(ccc4cccnc43)c2)cc(-c2cnc3c(n2)-c2ccccc2C32c3ccccc3Oc3ccccc32)c1] |
| 72 | 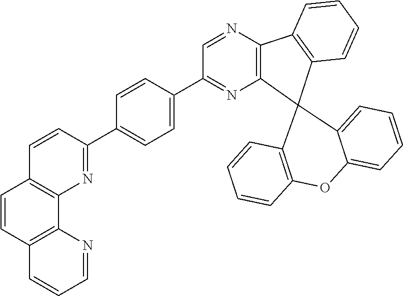 [SMILES: c1ccc2c(c1)Oc1ccccc1C21c2ccccc2-c2ncc(-c3ccc(-c4ccc5ccc6cccnc6c5n4)cc3)nc21] |
| 73 | 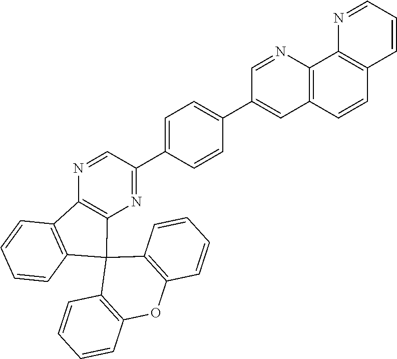 [SMILES: c1ccc2c(c1)Oc1ccccc1C21c2ccccc2-c2ncc(-c3ccc(-c4cnc5c(ccc6cccnc65)c4)cc3)nc21] |
| 74 | 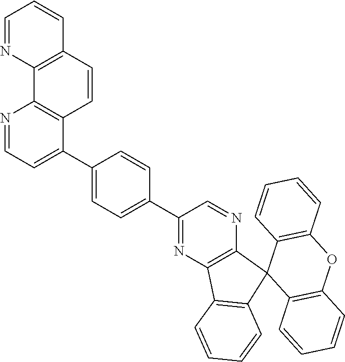 [SMILES: c1ccc2c(c1)Oc1ccccc1C21c2ccccc2-c2nc(-c3ccc(-c4ccnc5c4ccc4cccnc45)cc3)cnc21] |
| 75 | 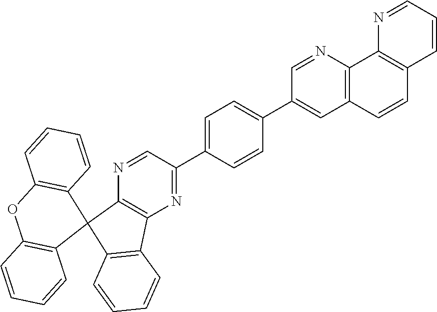 [SMILES: c1ccc2c(c1)Oc1ccccc1C21c2ccccc2-c2nc(-c3ccc(-c4cnc5c(ccc6cccnc65)c4)cc3)cnc21] |
| 76 | 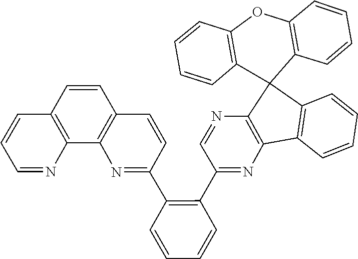 [SMILES: c1ccc2c(c1)Oc1ccccc1C21c2ccccc2-c2nc(-c3ccccc3-c3ccc4ccc5cccnc5c4n3)cnc21] |
| 77 | 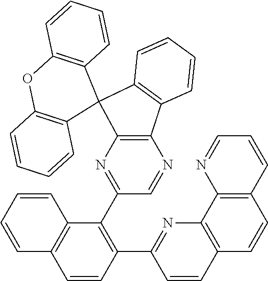 [SMILES: c1ccc2c(c1)Oc1ccccc1C21c2ccccc2-c2ncc(-c3c(-c4ccc5ccc6cccnc6c5n4)ccc4ccccc34)nc21] |
| 78 | 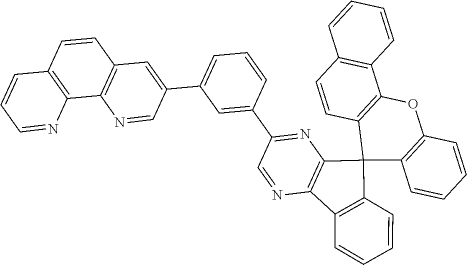 [SMILES: c1cc(-c2cnc3c(ccc4cccnc43)c2)cc(-c2cnc3c(n2)C2(c4ccccc4Oc4c2ccc2ccccc42)c2ccccc2-3)c1] |
| 79 | 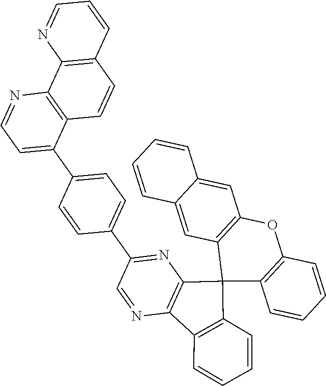 [SMILES: c1ccc2c(c1)Oc1cc3ccccc3cc1C21c2ccccc2-c2ncc(-c3ccc(-c4ccnc5c4ccc4cccnc45)cc3)nc21] |
| 80 | 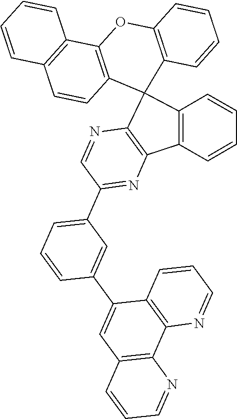 [SMILES: c1cc(-c2cnc3c(n2)-c2ccccc2C32c3ccccc3Oc3c2ccc2ccccc32)cc(-c2cc3cccnc3c3ncccc23)c1] |
| 81 | 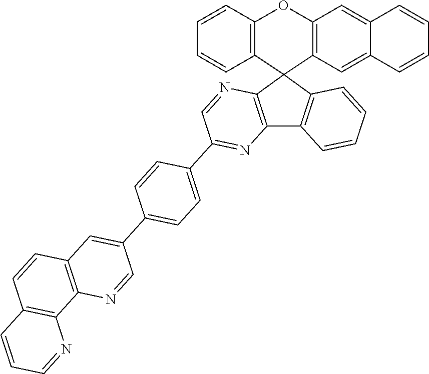 [SMILES: c1ccc2c(c1)Oc1cc3ccccc3cc1C21c2ccccc2-c2nc(-c3ccc(-c4cnc5c(ccc6cccnc65)c4)cc3)cnc21] |
| 82 | 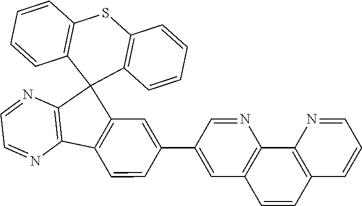 [SMILES: c1ccc2c(c1)Sc1ccccc1C21c2cc(-c3cnc4c(ccc5cccnc54)c3)ccc2-c2nccnc21] |
| 83 | 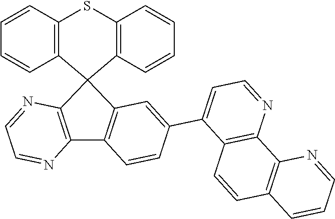 [SMILES: c1ccc2c(c1)Sc1ccccc1C21c2cc(-c3ccnc4c3ccc3cccnc34)ccc2-c2nccnc21] |
| 84 | 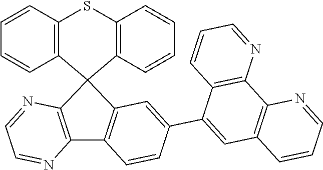 [SMILES: c1ccc2c(c1)Sc1ccccc1C21c2cc(-c3cc4cccnc4c4ncccc34)ccc2-c2nccnc21] |
| 85 | 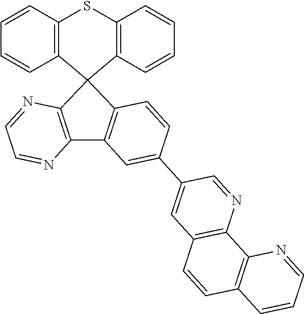 [SMILES: c1ccc2c(c1)Sc1ccccc1C21c2ccc(-c3cnc4c(ccc5cccnc54)c3)cc2-c2nccnc21] |
| 86 | 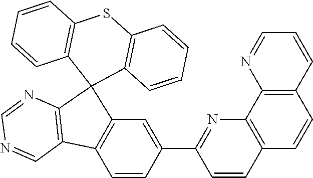 [SMILES: c1ccc2c(c1)Sc1ccccc1C21c2cc(-c3ccc4ccc5cccnc5c4n3)ccc2-c2cncnc21] |
| 87 | 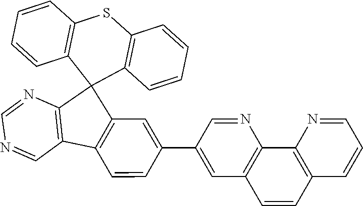 [SMILES: c1ccc2c(c1)Sc1ccccc1C21c2cc(-c3cnc4c(ccc5cccnc54)c3)ccc2-c2cncnc21] |
| 88 | 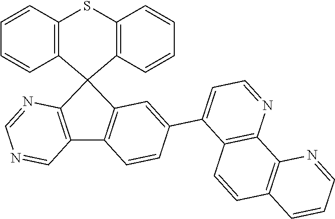 [SMILES: c1ccc2c(c1)Sc1ccccc1C21c2cc(-c3ccnc4c3ccc3cccnc34)ccc2-c2cncnc21] |
| 89 | 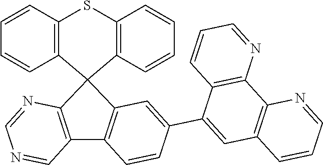 [SMILES: c1ccc2c(c1)Sc1ccccc1C21c2cc(-c3cc4cccnc4c4ncccc34)ccc2-c2cncnc21] |
| 90 | 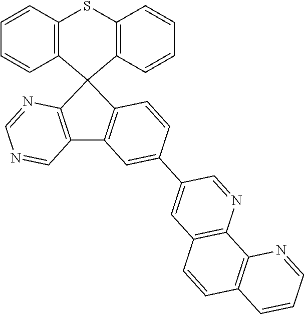 [SMILES: c1ccc2c(c1)Sc1ccccc1C21c2ccc(-c3cnc4c(ccc5cccnc54)c3)cc2-c2cncnc21] |
| 91 | 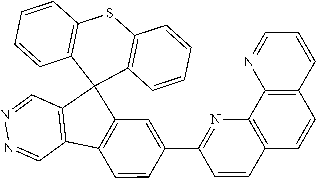 [SMILES: c1ccc2c(c1)Sc1ccccc1C21c2cnncc2-c2ccc(-c3ccc4ccc5cccnc5c4n3)cc21] |
| 92 | 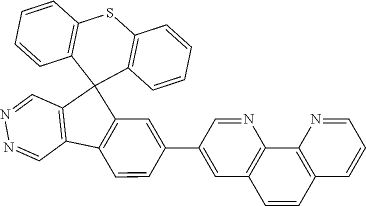 [SMILES: c1ccc2c(c1)Sc1ccccc1C21c2cnncc2-c2ccc(-c3cnc4c(ccc5cccnc54)c3)cc21] |
| 93 | 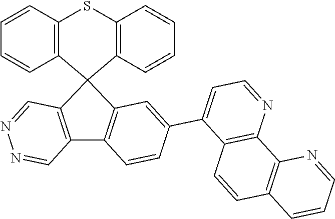 [SMILES: c1ccc2c(c1)Sc1ccccc1C21c2cnncc2-c2ccc(-c3ccnc4c3ccc3cccnc34)cc21] |
| 94 | 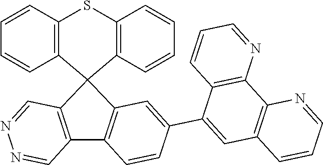 [SMILES: c1ccc2c(c1)Sc1ccccc1C21c2cnncc2-c2ccc(-c3cc4cccnc4c4ncccc34)cc21] |
| 95 | 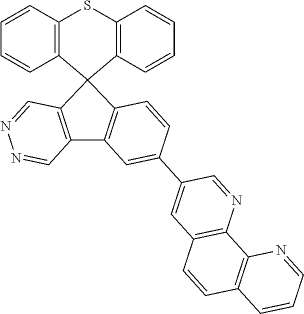 [SMILES: c1ccc2c(c1)Sc1ccccc1C21c2ccc(-c3cnc4c(ccc5cccnc54)c3)cc2-c2cnncc21] |
| 96 | 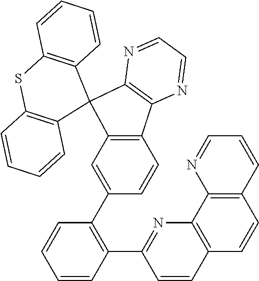 [SMILES: c1ccc2c(c1)Sc1ccccc1C21c2cc(-c3ccccc3-c3ccc4ccc5cccnc5c4n3)ccc2-c2nccnc21] |
| 97 | 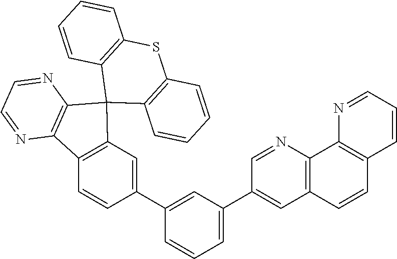 [SMILES: c1cc(-c2ccc3c(c2)C2(c4ccccc4Sc4ccccc42)c2nccnc2-3)cc(-c2cnc3c(ccc4cccnc43)c2)c1] |
| 98 | 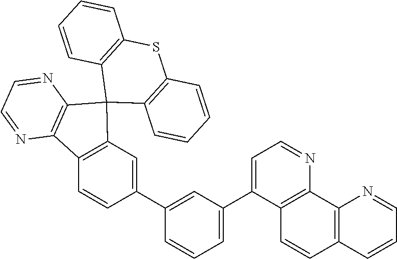 [SMILES: c1cc(-c2ccc3c(c2)C2(c4ccccc4Sc4ccccc42)c2nccnc2-3)cc(-c2ccnc3c2ccc2cccnc23)c1] |
| 99 | 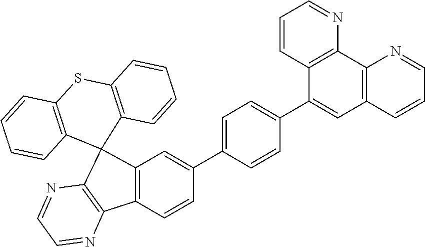 [SMILES: c1ccc2c(c1)Sc1ccccc1C21c2cc(-c3ccc(-c4cc5cccnc5c5ncccc45)cc3)ccc2-c2nccnc21] |
| 100 | 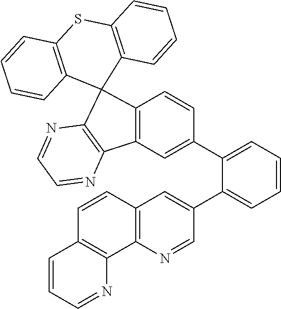 [SMILES: c1ccc2c(c1)Sc1ccccc1C21c2ccc(-c3ccccc3-c3cnc4c(ccc5cccnc54)c3)cc2-c2nccnc21] |
| 101 | 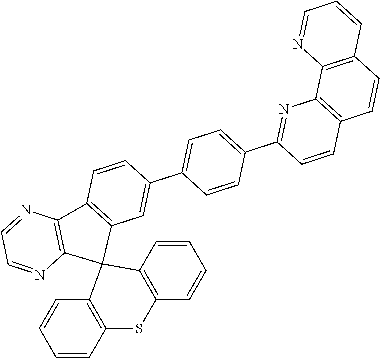 [SMILES: c1ccc2c(c1)Sc1ccccc1C21c2cc(-c3ccc(-c4ccc5ccc6cccnc6c5n4)cc3)ccc2-c2nccnc21] |
| 102 | 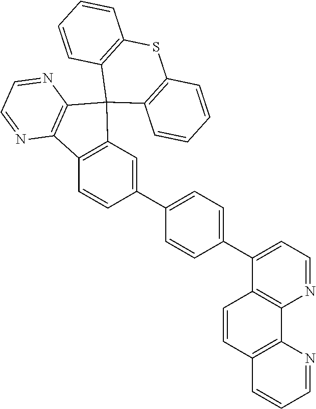 [SMILES: c1ccc2c(c1)Sc1ccccc1C21c2cc(-c3ccc(-c4ccnc5c4ccc4cccnc45)cc3)ccc2-c2nccnc21] |
| 103 | 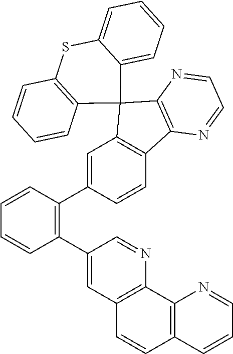 [SMILES: c1ccc2c(c1)Sc1ccccc1C21c2cc(-c3ccccc3-c3cnc4c(ccc5cccnc54)c3)ccc2-c2nccnc21] |
| 104 | 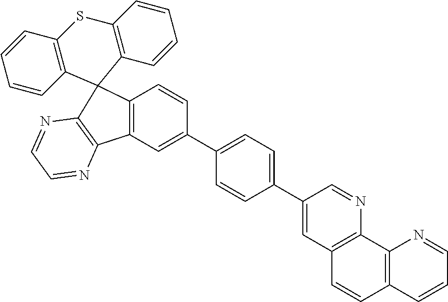 [SMILES: c1ccc2c(c1)Sc1ccccc1C21c2ccc(-c3ccc(-c4cnc5c(ccc6cccnc65)c4)cc3)cc2-c2nccnc21] |
| 105 | 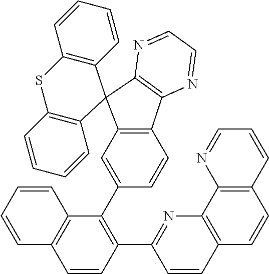 [SMILES: c1ccc2c(c1)Sc1ccccc1C21c2cc(-c3c(-c4ccc5ccc6cccnc6c5n4)ccc4ccccc34)ccc2-c2nccnc21] |
| 106 | 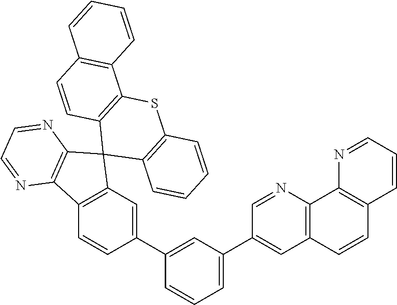 [SMILES: c1cc(-c2ccc3c(c2)C2(c4ccccc4Sc4c2ccc2ccccc42)c2nccnc2-3)cc(-c2cnc3c(ccc4cccnc43)c2)c1] |
| 107 | 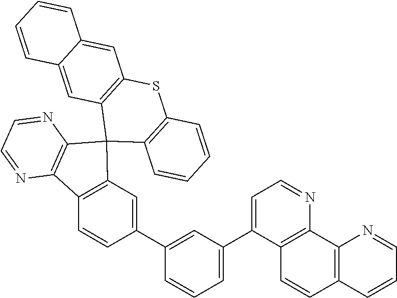 [SMILES: c1cc(-c2ccc3c(c2)C2(c4ccccc4Sc4cc5ccccc5cc42)c2nccnc2-3)cc(-c2ccnc3c2ccc2cccnc23)c1] |
| 108 | 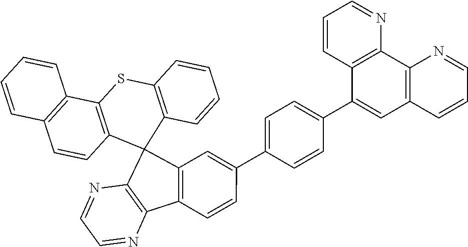 [SMILES: c1ccc2c(c1)Sc1c(ccc3ccccc13)C21c2cc(-c3ccc(-c4cc5cccnc5c5ncccc45)cc3)ccc2-c2nccnc21] |
| 109 | 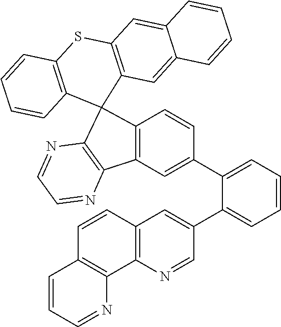 [SMILES: c1ccc2c(c1)Sc1cc3ccccc3cc1C21c2ccc(-c3ccccc3-c3cnc4c(ccc5cccnc54)c3)cc2-c2nccnc21] |
| 110 | 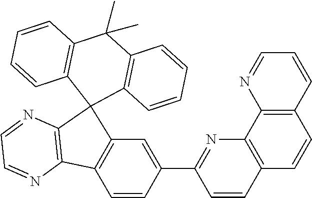 [SMILES: CC1(C)c2ccccc2C2(c3cc(-c4ccc5ccc6cccnc6c5n4)ccc3-c3nccnc32)c2ccccc21] |
| 111 | 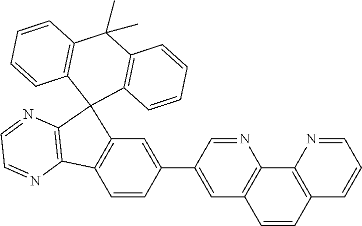 [SMILES: CC1(C)c2ccccc2C2(c3cc(-c4cnc5c(ccc6cccnc65)c4)ccc3-c3nccnc32)c2ccccc21] |
| 112 | 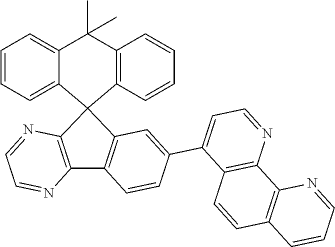 [SMILES: CC1(C)c2ccccc2C2(c3cc(-c4ccnc5c4ccc4cccnc45)ccc3-c3nccnc32)c2ccccc21] |
| 113 | 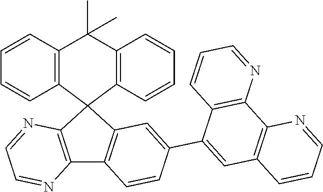 [SMILES: CC1(C)c2ccccc2C2(c3cc(-c4cc5cccnc5c5ncccc45)ccc3-c3nccnc32)c2ccccc21] |
| 114 | 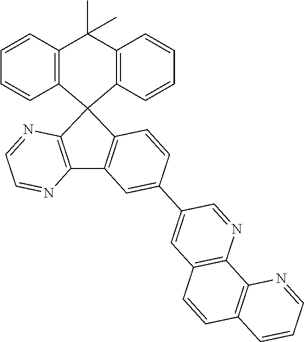 [SMILES: CC1(C)c2ccccc2C2(c3ccc(-c4cnc5c(ccc6cccnc65)c4)cc3-c3nccnc32)c2ccccc21] |
| 115 | 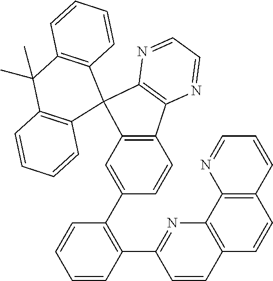 [SMILES: CC1(C)c2ccccc2C2(c3cc(-c4ccccc4-c4ccc5ccc6cccnc6c5n4)ccc3-c3nccnc32)c2ccccc21] |
| 116 | 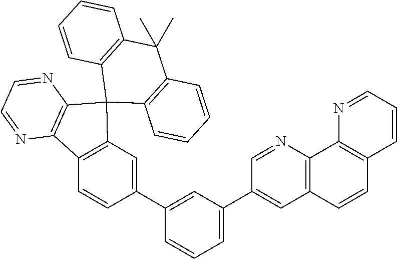 [SMILES: CC1(C)c2ccccc2C2(c3cc(-c4cccc(-c5cnc6c(ccc7cccnc76)c5)c4)ccc3-c3nccnc32)c2ccccc21] |
| 117 | 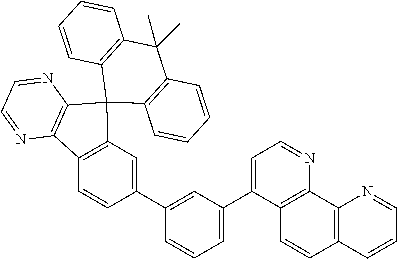 [SMILES: CC1(C)c2ccccc2C2(c3cc(-c4cccc(-c5ccnc6c5ccc5cccnc56)c4)ccc3-c3nccnc32)c2ccccc21] |
| 118 | 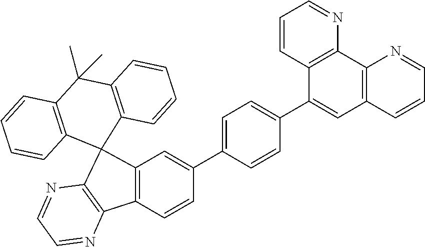 [SMILES: CC1(C)c2ccccc2C2(c3cc(-c4ccc(-c5cc6cccnc6c6ncccc56)cc4)ccc3-c3nccnc32)c2ccccc21] |
| 119 | 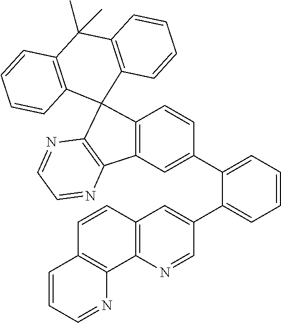 [SMILES: CC1(C)c2ccccc2C2(c3ccc(-c4ccccc4-c4cnc5c(ccc6cccnc65)c4)cc3-c3nccnc32)c2ccccc21] |
| 120 | 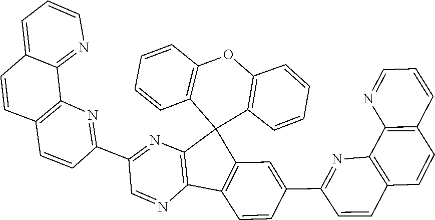 [SMILES: c1ccc2c(c1)Oc1ccccc1C21c2cc(-c3ccc4ccc5cccnc5c4n3)ccc2-c2ncc(-c3ccc4ccc5cccnc5c4n3)nc21] |
| 121 | 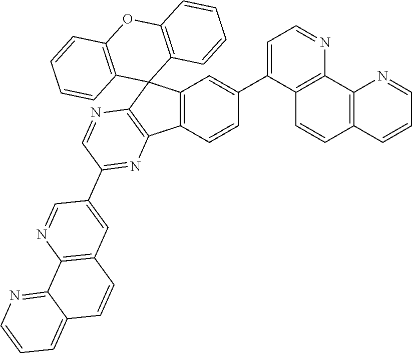 [SMILES: c1ccc2c(c1)Oc1ccccc1C21c2cc(-c3ccnc4c3ccc3cccnc34)ccc2-c2nc(-c3cnc4c(ccc5cccnc54)c3)cnc21] |
| 122 | 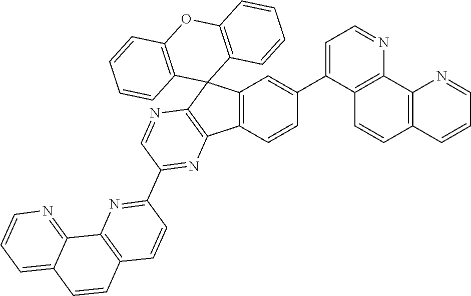 [SMILES: c1ccc2c(c1)Oc1ccccc1C21c2cc(-c3ccnc4c3ccc3cccnc34)ccc2-c2nc(-c3ccc4ccc5cccnc5c4n3)cnc21] |
| 123 | 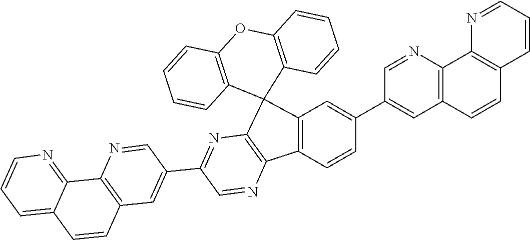 [SMILES: c1ccc2c(c1)Oc1ccccc1C21c2cc(-c3cnc4c(ccc5cccnc54)c3)ccc2-c2ncc(-c3cnc4c(ccc5cccnc54)c3)nc21] |
| 124 | 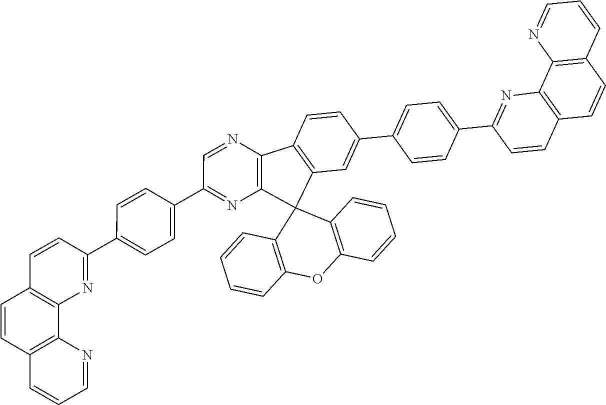 [SMILES: c1ccc2c(c1)Oc1ccccc1C21c2cc(-c3ccc(-c4ccc5ccc6cccnc6c5n4)cc3)ccc2-c2ncc(-c3ccc(-c4ccc5ccc6cccnc6c5n4)cc3)nc21] |
| 125 | 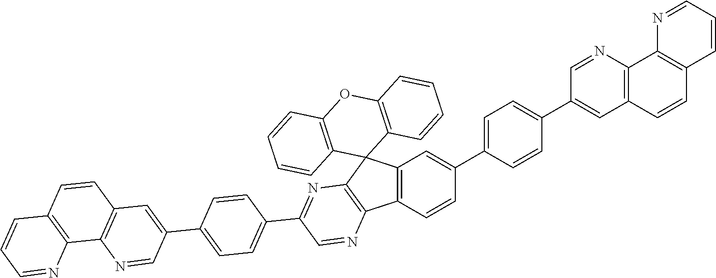 [SMILES: c1ccc2c(c1)Oc1ccccc1C21c2cc(-c3ccc(-c4cnc5c(ccc6cccnc65)c4)cc3)ccc2-c2ncc(-c3ccc(-c4cnc5c(ccc6cccnc65)c4)cc3)nc21] |
| 126 | 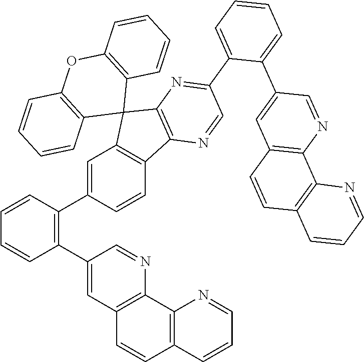 [SMILES: c1ccc2c(c1)Oc1ccccc1C21c2cc(-c3ccccc3-c3cnc4c(ccc5cccnc54)c3)ccc2-c2ncc(-c3ccccc3-c3cnc4c(ccc5cccnc54)c3)nc21] |
| 127 | 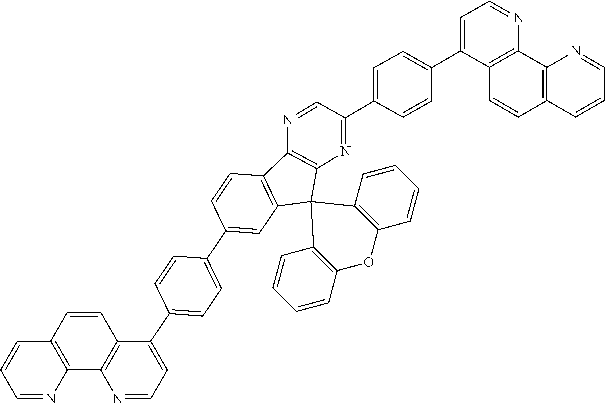 [SMILES: c1ccc2c(c1)Oc1ccccc1C21c2cc(-c3ccc(-c4ccnc5c4ccc4cccnc45)cc3)ccc2-c2ncc(-c3ccc(-c4ccnc5c4ccc4cccnc45)cc3)nc21] |
| 128 | 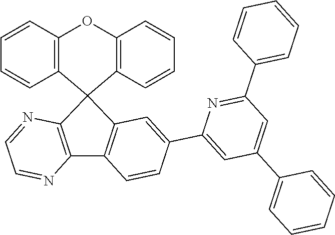 [SMILES: c1ccc(-c2cc(-c3ccccc3)nc(-c3ccc4c(c3)C3(c5ccccc5Oc5ccccc53)c3nccnc3-4)c2)cc1] |
| 129 | 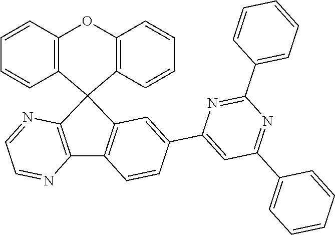 [SMILES: c1ccc(-c2cc(-c3ccc4c(c3)C3(c5ccccc5Oc5ccccc53)c3nccnc3-4)nc(-c3ccccc3)n2)cc1] |
| 130 | 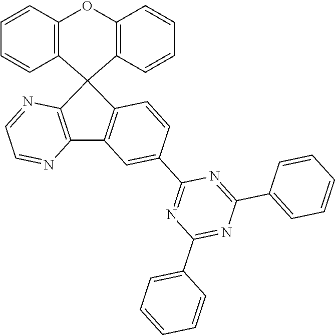 [SMILES: c1ccc(-c2nc(-c3ccccc3)nc(-c3ccc4c(c3)-c3nccnc3C43c4ccccc4Oc4ccccc43)n2)cc1] |
| 131 | 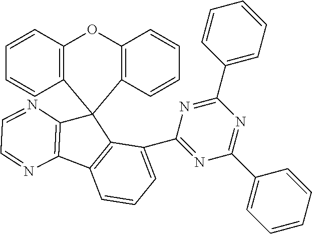 [SMILES: c1ccc(-c2nc(-c3ccccc3)nc(-c3cccc4c3C3(c5ccccc5Oc5ccccc53)c3nccnc3-4)n2)cc1] |
| 132 | 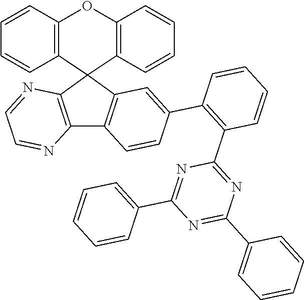 [SMILES: c1ccc(-c2nc(-c3ccccc3)nc(-c3ccccc3-c3ccc4c(c3)C3(c5ccccc5Oc5ccccc53)c3nccnc3-4)n2)cc1] |
| 133 | 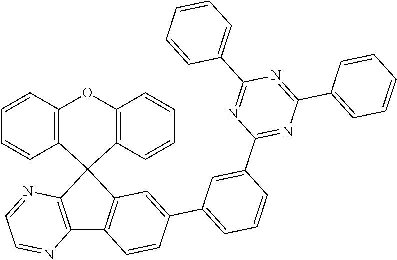 [SMILES: c1ccc(-c2nc(-c3ccccc3)nc(-c3cccc(-c4ccc5c(c4)C4(c6ccccc6Oc6ccccc64)c4nccnc4-5)c3)n2)cc1] |
| 134 | 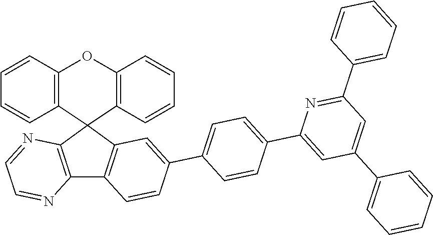 [SMILES: c1ccc(-c2cc(-c3ccccc3)nc(-c3ccc(-c4ccc5c(c4)C4(c6ccccc6Oc6ccccc64)c4nccnc4-5)cc3)c2)cc1] |
| 135 | 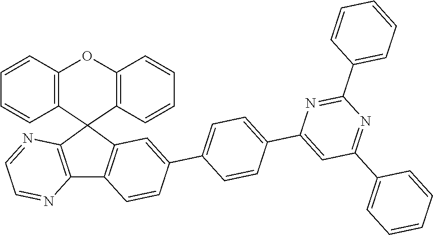 [SMILES: c1ccc(-c2cc(-c3ccc(-c4ccc5c(c4)C4(c6ccccc6Oc6ccccc64)c4nccnc4-5)cc3)nc(-c3ccccc3)n2)cc1] |
| 136 | 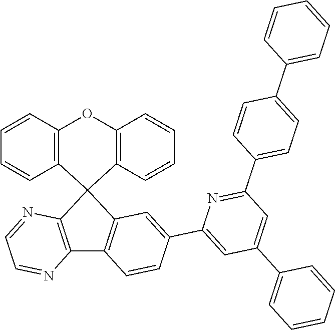 [SMILES: c1ccc(-c2ccc(-c3cc(-c4ccccc4)cc(-c4ccc5c(c4)C4(c6ccccc6Oc6ccccc64)c4nccnc4-5)n3)cc2)cc1] |
| 137 | 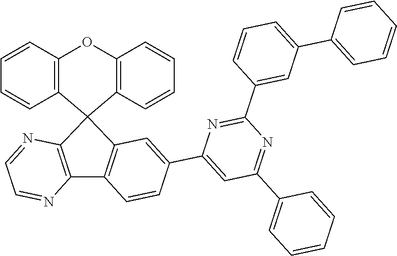 [SMILES: c1ccc(-c2cccc(-c3nc(-c4ccccc4)cc(-c4ccc5c(c4)C4(c6ccccc6Oc6ccccc64)c4nccnc4-5)n3)c2)cc1] |
| 138 | 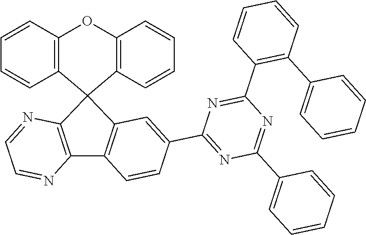 [SMILES: c1ccc(-c2nc(-c3ccc4c(c3)C3(c5ccccc5Oc5ccccc53)c3nccnc3-4)nc(-c3ccccc3-c3ccccc3)n2)cc1] |
| 139 | 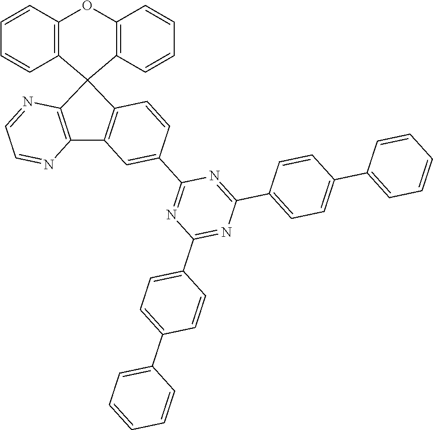 [SMILES: c1ccc(-c2ccc(-c3nc(-c4ccc(-c5ccccc5)cc4)nc(-c4ccc5c(c4)-c4nccnc4C54c5ccccc5Oc5ccccc54)n3)cc2)cc1] |
| 140 | 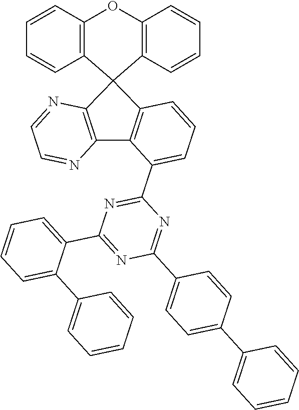 [SMILES: c1ccc(-c2ccc(-c3nc(-c4ccccc4-c4ccccc4)nc(-c4cccc5c4-c4nccnc4C54c5ccccc5Oc5ccccc54)n3)cc2)cc1] |
| 141 | 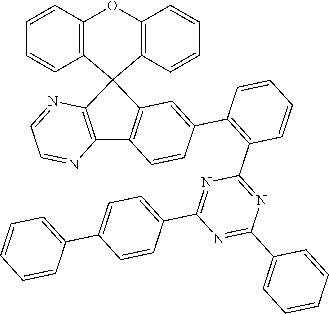 [SMILES: c1ccc(-c2ccc(-c3nc(-c4ccccc4)nc(-c4ccccc4-c4ccc5c(c4)C4(c6ccccc6Oc6ccccc64)c4nccnc4-5)n3)cc2)cc1] |
| 142 | 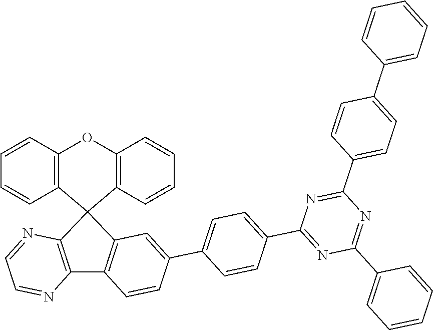 [SMILES: c1ccc(-c2ccc(-c3nc(-c4ccccc4)nc(-c4ccc(-c5ccc6c(c5)C5(c7ccccc7Oc7ccccc75)c5nccnc5-6)cc4)n3)cc2)cc1] |
| 143 | 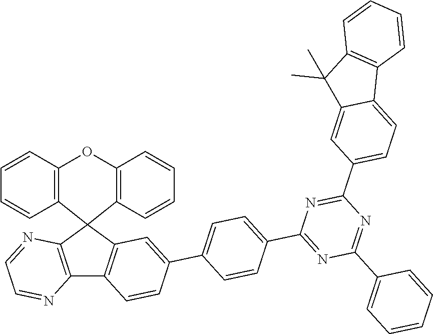 [SMILES: CC1(C)c2ccccc2-c2ccc(-c3nc(-c4ccccc4)nc(-c4ccc(-c5ccc6c(c5)C5(c7ccccc7Oc7ccccc75)c5nccnc5-6)cc4)n3)cc21] |
| 144 | 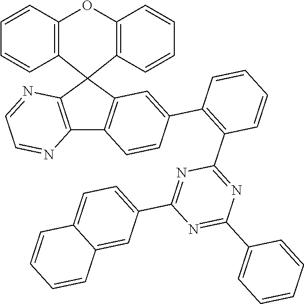 [SMILES: c1ccc(-c2nc(-c3ccc4ccccc4c3)nc(-c3ccccc3-c3ccc4c(c3)C3(c5ccccc5Oc5ccccc53)c3nccnc3-4)n2)cc1] |
| 145 | 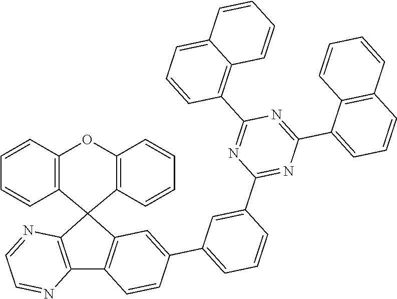 [SMILES: c1cc(-c2ccc3c(c2)C2(c4ccccc4Oc4ccccc42)c2nccnc2-3)cc(-c2nc(-c3cccc4ccccc34)nc(-c3cccc4ccccc34)n2)c1] |
| 146 | 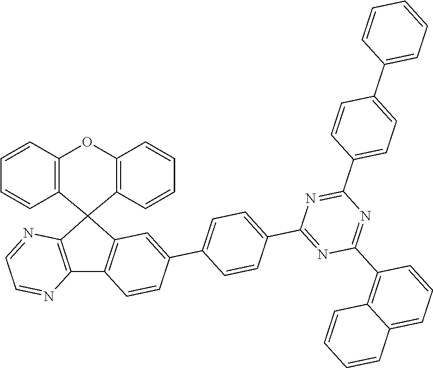 [SMILES: c1ccc(-c2ccc(-c3nc(-c4ccc(-c5ccc6c(c5)C5(c7ccccc7Oc7ccccc75)c5nccnc5-6)cc4)nc(-c4cccc5ccccc45)n3)cc2)cc1] |
| 147 | 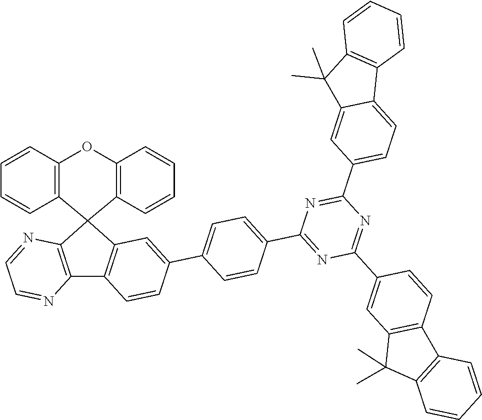 [SMILES: CC1(C)c2ccccc2-c2ccc(-c3nc(-c4ccc(-c5ccc6c(c5)C5(c7ccccc7Oc7ccccc75)c5nccnc5-6)cc4)nc(-c4ccc5c(c4)C(C)(C)c4ccccc4-5)n3)cc21] |
| 148 | 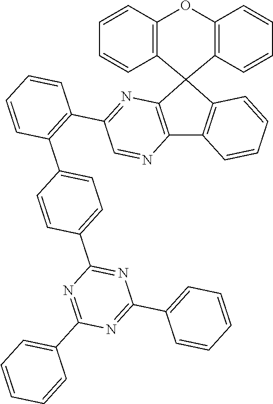 [SMILES: c1ccc(-c2nc(-c3ccccc3)nc(-c3ccc(-c4ccccc4-c4cnc5c(n4)C4(c6ccccc6Oc6ccccc64)c4ccccc4-5)cc3)n2)cc1] |
| 149 | 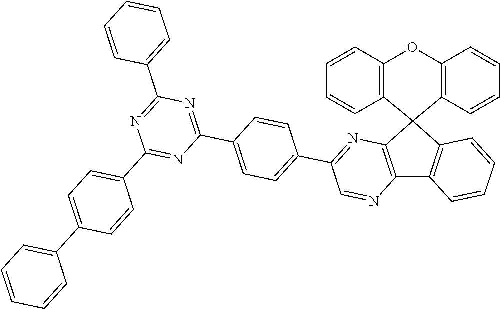 [SMILES: c1ccc(-c2ccc(-c3nc(-c4ccccc4)nc(-c4ccc(-c5cnc6c(n5)C5(c7ccccc7Oc7ccccc75)c5ccccc5-6)cc4)n3)cc2)cc1] |
| 150 | 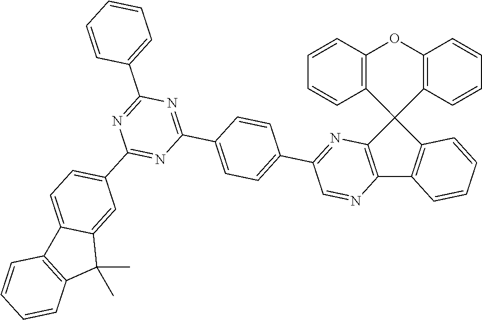 [SMILES: CC1(C)c2ccccc2-c2ccc(-c3nc(-c4ccccc4)nc(-c4ccc(-c5cnc6c(n5)C5(c7ccccc7Oc7ccccc75)c5ccccc5-6)cc4)n3)cc21] |
| 151 | 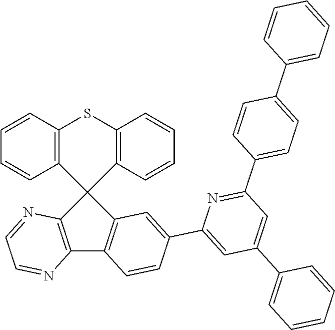 [SMILES: c1ccc(-c2ccc(-c3cc(-c4ccccc4)cc(-c4ccc5c(c4)C4(c6ccccc6Sc6ccccc64)c4nccnc4-5)n3)cc2)cc1] |
| 152 | 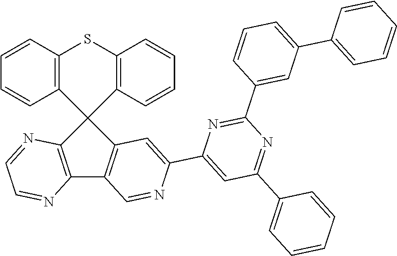 [SMILES: c1ccc(-c2cccc(-c3nc(-c4ccccc4)cc(-c4cc5c(cn4)-c4nccnc4C54c5ccccc5Sc5ccccc54)n3)c2)cc1] |
| 153 | 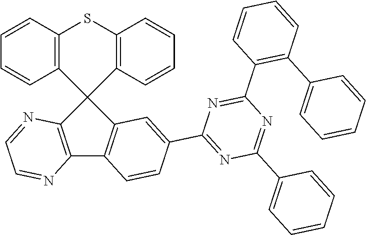 [SMILES: c1ccc(-c2nc(-c3ccc4c(c3)C3(c5ccccc5Sc5ccccc53)c3nccnc3-4)nc(-c3ccccc3-c3ccccc3)n2)cc1] |
| 154 | 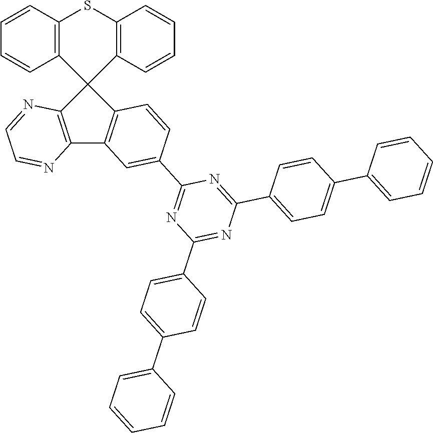 [SMILES: c1ccc(-c2ccc(-c3nc(-c4ccc(-c5ccccc5)cc4)nc(-c4ccc5c(c4)-c4nccnc4C54c5ccccc5Sc5ccccc54)n3)cc2)cc1] |
| 155 | 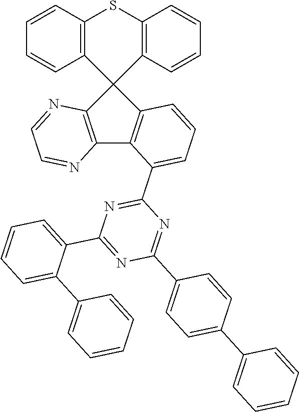 [SMILES: c1ccc(-c2ccc(-c3nc(-c4ccccc4-c4ccccc4)nc(-c4cccc5c4-c4nccnc4C54c5ccccc5Sc5ccccc54)n3)cc2)cc1] |
| 156 | 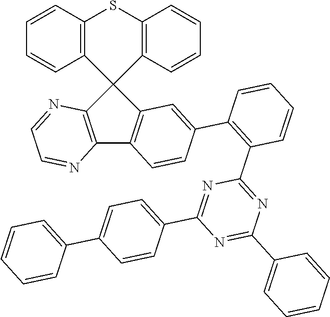 [SMILES: c1ccc(-c2ccc(-c3nc(-c4ccccc4)nc(-c4ccccc4-c4ccc5c(c4)C4(c6ccccc6Sc6ccccc64)c4nccnc4-5)n3)cc2)cc1] |
| 157 | 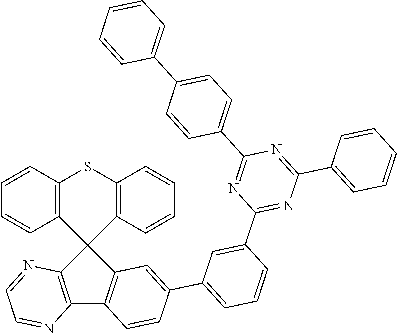 [SMILES: c1ccc(-c2ccc(-c3nc(-c4ccccc4)nc(-c4cccc(-c5ccc6c(c5)C5(c7ccccc7Sc7ccccc75)c5nccnc5-6)c4)n3)cc2)cc1] |
| 158 | 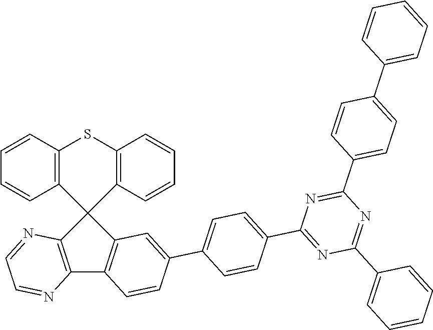 [SMILES: c1ccc(-c2ccc(-c3nc(-c4ccccc4)nc(-c4ccc(-c5ccc6c(c5)C5(c7ccccc7Sc7ccccc75)c5nccnc5-6)cc4)n3)cc2)cc1] |
| 159 | 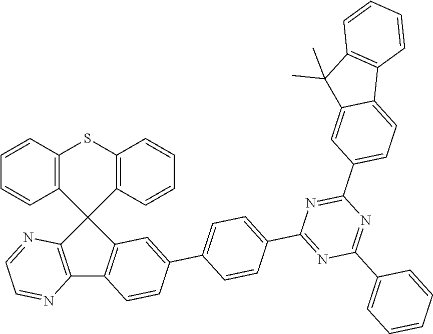 [SMILES: CC1(C)c2ccccc2-c2ccc(-c3nc(-c4ccccc4)nc(-c4ccc(-c5ccc6c(c5)C5(c7ccccc7Sc7ccccc75)c5nccnc5-6)cc4)n3)cc21] |
| 160 | 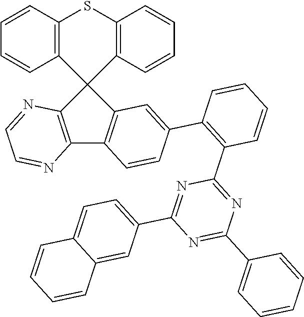 [SMILES: c1ccc(-c2nc(-c3ccc4ccccc4c3)nc(-c3ccccc3-c3ccc4c(c3)C3(c5ccccc5Sc5ccccc53)c3nccnc3-4)n2)cc1] |
| 161 | 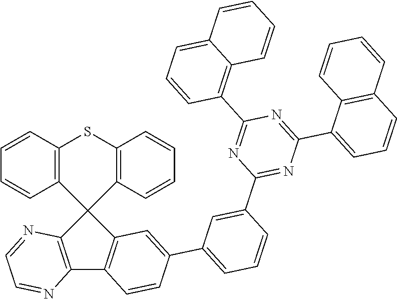 [SMILES: c1cc(-c2ccc3c(c2)C2(c4ccccc4Sc4ccccc42)c2nccnc2-3)cc(-c2nc(-c3cccc4ccccc34)nc(-c3cccc4ccccc34)n2)c1] |
| 162 | 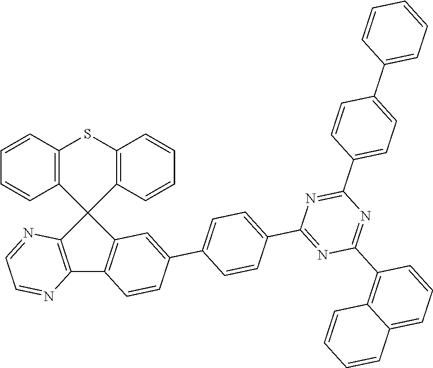 [SMILES: c1ccc(-c2ccc(-c3nc(-c4ccc(-c5ccc6c(c5)C5(c7ccccc7Sc7ccccc75)c5nccnc5-6)cc4)nc(-c4cccc5ccccc45)n3)cc2)cc1] |
| 163 | 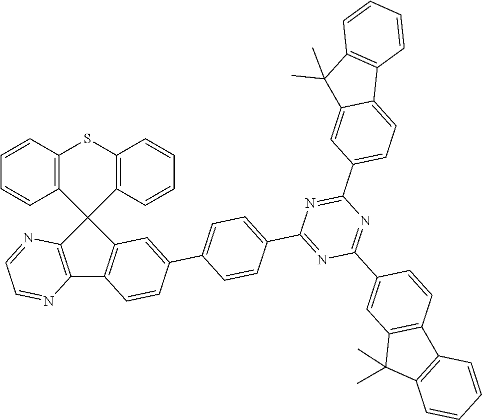 [SMILES: CC1(C)c2ccccc2-c2ccc(-c3nc(-c4ccc(-c5ccc6c(c5)C5(c7ccccc7Sc7ccccc75)c5nccnc5-6)cc4)nc(-c4ccc5c(c4)C(C)(C)c4ccccc4-5)n3)cc21] |
| 164 | 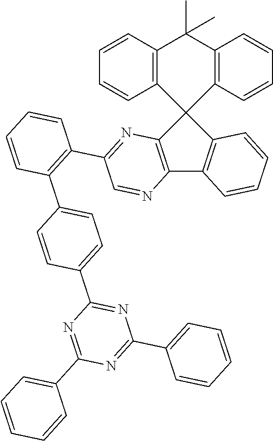 [SMILES: CC1(C)c2ccccc2C2(c3ccccc3-c3ncc(-c4ccccc4-c4ccc(-c5nc(-c6ccccc6)nc(-c6ccccc6)n5)cc4)nc32)c2ccccc21] |
| 165 | 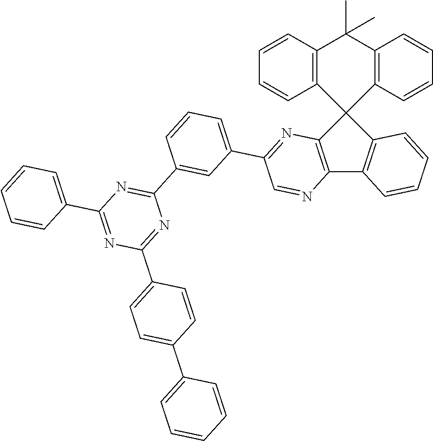 [SMILES: CC1(C)c2ccccc2C2(c3ccccc3-c3ncc(-c4cccc(-c5nc(-c6ccccc6)nc(-c6ccc(-c7ccccc7)cc6)n5)c4)nc32)c2ccccc21] |
| 166 | 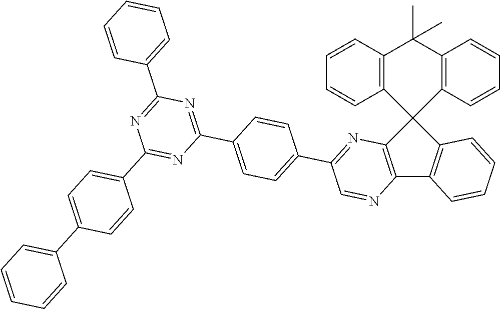 [SMILES: CC1(C)c2ccccc2C2(c3ccccc3-c3ncc(-c4ccc(-c5nc(-c6ccccc6)nc(-c6ccc(-c7ccccc7)cc6)n5)cc4)nc32)c2ccccc21] |
| 167 | 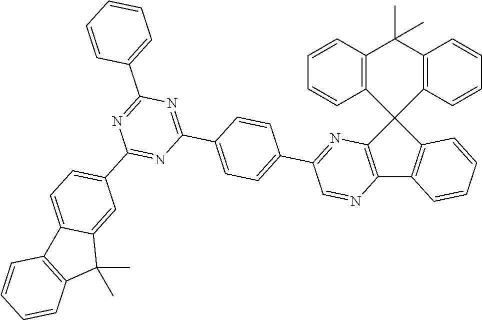 [SMILES: CC1(C)c2ccccc2-c2ccc(-c3nc(-c4ccccc4)nc(-c4ccc(-c5cnc6c(n5)C5(c7ccccc7-6)c6ccccc6C(C)(C)c6ccccc65)cc4)n3)cc21] |
| 168 | 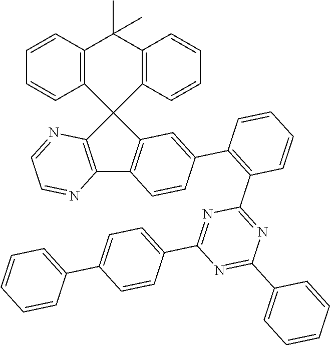 [SMILES: CC1(C)c2ccccc2C2(c3cc(-c4ccccc4-c4nc(-c5ccccc5)nc(-c5ccc(-c6ccccc6)cc5)n4)ccc3-c3nccnc32)c2ccccc21] |
| 169 | 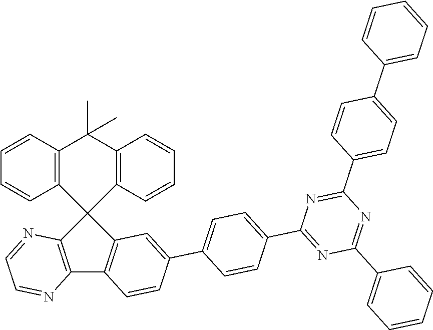 [SMILES: CC1(C)c2ccccc2C2(c3cc(-c4ccc(-c5nc(-c6ccccc6)nc(-c6ccc(-c7ccccc7)cc6)n5)cc4)ccc3-c3nccnc32)c2ccccc21] |
| 170 | 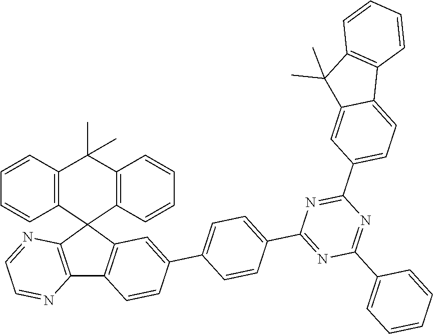 [SMILES: CC1(C)c2ccccc2-c2ccc(-c3nc(-c4ccccc4)nc(-c4ccc(-c5ccc6c(c5)C5(c7ccccc7C(C)(C)c7ccccc75)c5nccnc5-6)cc4)n3)cc21] |
| 171 | 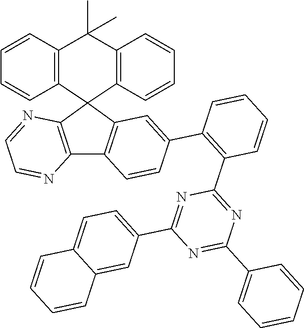 [SMILES: CC1(C)c2ccccc2C2(c3cc(-c4ccccc4-c4nc(-c5ccccc5)nc(-c5ccc6ccccc6c5)n4)ccc3-c3nccnc32)c2ccccc21] |
| 172 | 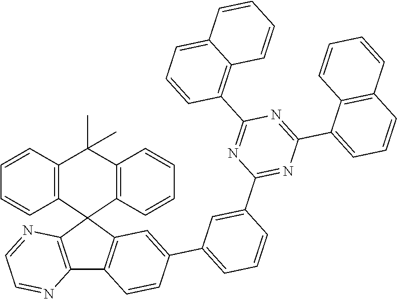 [SMILES: CC1(C)c2ccccc2C2(c3cc(-c4cccc(-c5nc(-c6cccc7ccccc67)nc(-c6cccc7ccccc67)n5)c4)ccc3-c3nccnc32)c2ccccc21] |
| 173 | 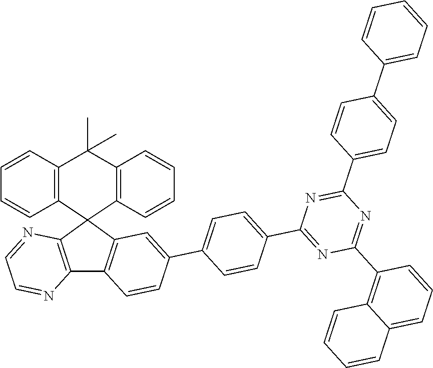 [SMILES: CC1(C)c2ccccc2C2(c3cc(-c4ccc(-c5nc(-c6ccc(-c7ccccc7)cc6)nc(-c6cccc7ccccc67)n5)cc4)ccc3-c3nccnc32)c2ccccc21] |
| 174 | 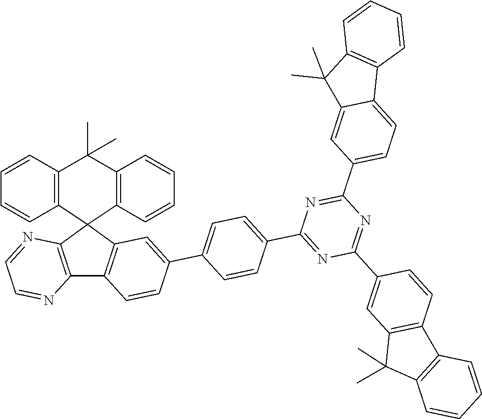 [SMILES: CC1(C)c2ccccc2-c2ccc(-c3nc(-c4ccc(-c5ccc6c(c5)C5(c7ccccc7C(C)(C)c7ccccc75)c5nccnc5-6)cc4)nc(-c4ccc5c(c4)C(C)(C)c4ccccc4-5)n3)cc21] |
| 175 | 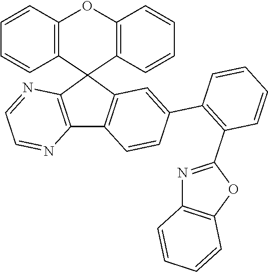 [SMILES: c1ccc2c(c1)Oc1ccccc1C21c2cc(-c3ccccc3-c3nc4ccccc4o3)ccc2-c2nccnc21] |
| 176 | 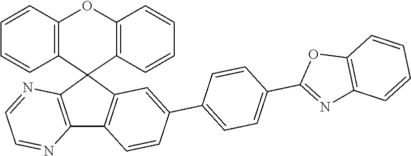 [SMILES: c1ccc2c(c1)Oc1ccccc1C21c2cc(-c3ccc(-c4nc5ccccc5o4)cc3)ccc2-c2nccnc21] |
| 177 | 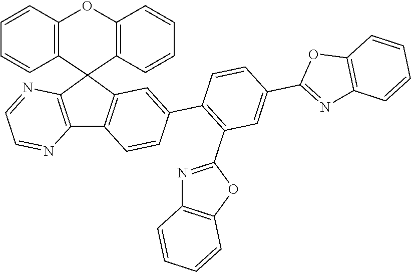 [SMILES: c1ccc2c(c1)Oc1ccccc1C21c2cc(-c3ccc(-c4nc5ccccc5o4)cc3-c3nc4ccccc4o3)ccc2-c2nccnc21] |
| 178 | 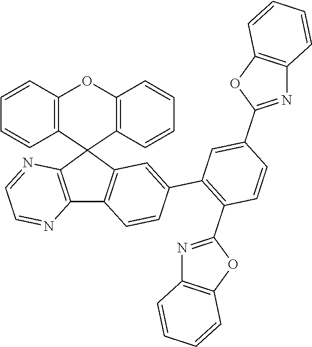 [SMILES: c1ccc2c(c1)Oc1ccccc1C21c2cc(-c3cc(-c4nc5ccccc5o4)ccc3-c3nc4ccccc4o3)ccc2-c2nccnc21] |
| 179 | 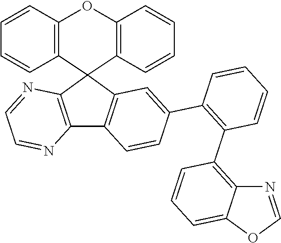 [SMILES: c1ccc2c(c1)Oc1ccccc1C21c2cc(-c3ccccc3-c3cccc4ocnc34)ccc2-c2nccnc21] |
| 180 | 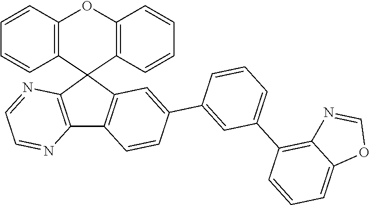 [SMILES: c1cc(-c2ccc3c(c2)C2(c4ccccc4Oc4ccccc42)c2nccnc2-3)cc(-c2cccc3ocnc23)c1] |
| 181 | 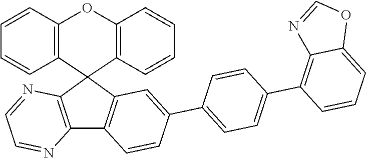 [SMILES: c1ccc2c(c1)Oc1ccccc1C21c2cc(-c3ccc(-c4cccc5ocnc45)cc3)ccc2-c2nccnc21] |
| 182 | 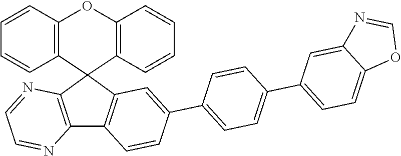 [SMILES: c1ccc2c(c1)Oc1ccccc1C21c2cc(-c3ccc(-c4ccc5ocnc5c4)cc3)ccc2-c2nccnc21] |
| 183 | 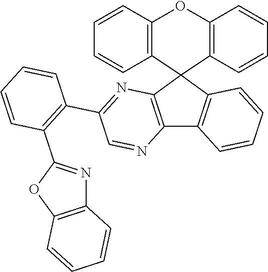 [SMILES: c1ccc2c(c1)Oc1ccccc1C21c2ccccc2-c2ncc(-c3ccccc3-c3nc4ccccc4o3)nc21] |
| 184 | 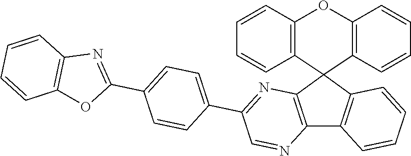 [SMILES: c1ccc2c(c1)Oc1ccccc1C21c2ccccc2-c2ncc(-c3ccc(-c4nc5ccccc5o4)cc3)nc21] |
| 185 | 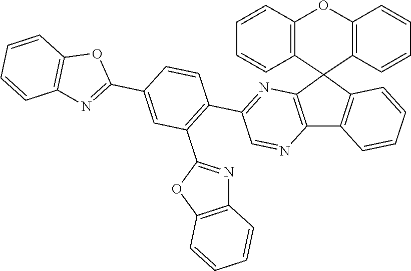 [SMILES: c1ccc2c(c1)Oc1ccccc1C21c2ccccc2-c2ncc(-c3ccc(-c4nc5ccccc5o4)cc3-c3nc4ccccc4o3)nc21] |
| 186 | 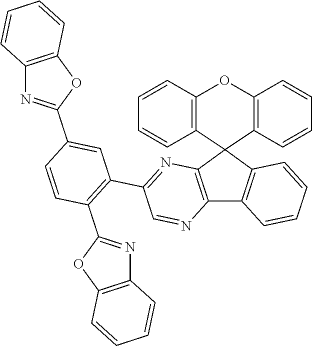 [SMILES: c1ccc2c(c1)Oc1ccccc1C21c2ccccc2-c2ncc(-c3cc(-c4nc5ccccc5o4)ccc3-c3nc4ccccc4o3)nc21] |
| 187 | 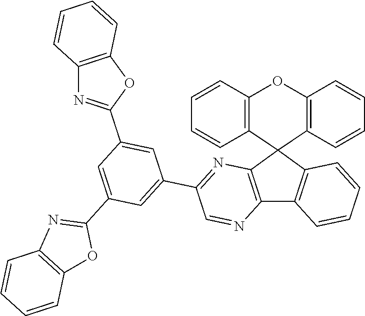 [SMILES: c1ccc2c(c1)Oc1ccccc1C21c2ccccc2-c2ncc(-c3cc(-c4nc5ccccc5o4)cc(-c4nc5ccccc5o4)c3)nc21] |
| 188 | 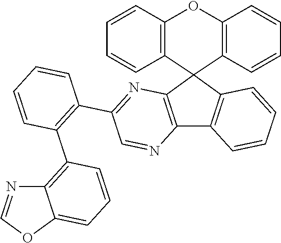 [SMILES: c1ccc2c(c1)Oc1ccccc1C21c2ccccc2-c2ncc(-c3ccccc3-c3cccc4ocnc34)nc21] |
| 189 | 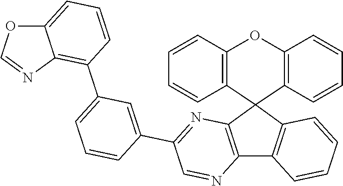 [SMILES: c1cc(-c2cnc3c(n2)C2(c4ccccc4Oc4ccccc42)c2ccccc2-3)cc(-c2cccc3ocnc23)c1] |
| 190 | 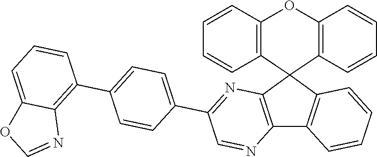 [SMILES: c1ccc2c(c1)Oc1ccccc1C21c2ccccc2-c2ncc(-c3ccc(-c4cccc5ocnc45)cc3)nc21] |
| 191 | 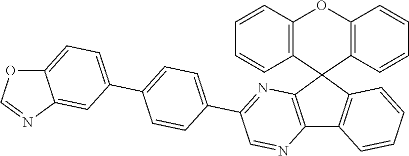 [SMILES: c1ccc2c(c1)Oc1ccccc1C21c2ccccc2-c2ncc(-c3ccc(-c4ccc5ocnc5c4)cc3)nc21] |
| 192 | 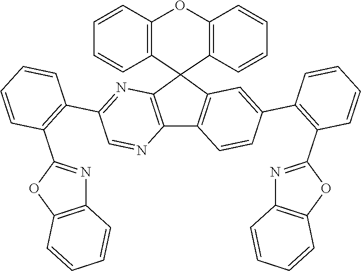 [SMILES: c1ccc2c(c1)Oc1ccccc1C21c2cc(-c3ccccc3-c3nc4ccccc4o3)ccc2-c2ncc(-c3ccccc3-c3nc4ccccc4o3)nc21] |
| 193 | 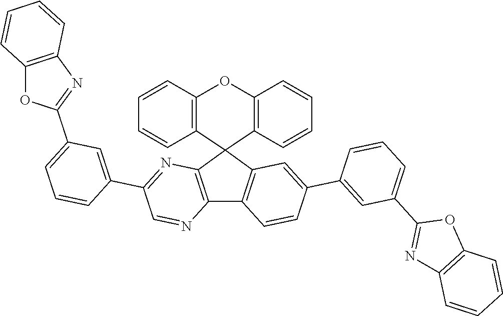 [SMILES: c1cc(-c2ccc3c(c2)C2(c4ccccc4Oc4ccccc42)c2nc(-c4cccc(-c5nc6ccccc6o5)c4)cnc2-3)cc(-c2nc3ccccc3o2)c1] |
| 194 | 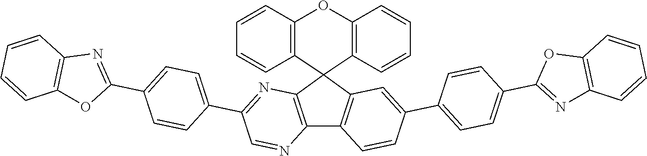 [SMILES: c1ccc2c(c1)Oc1ccccc1C21c2cc(-c3ccc(-c4nc5ccccc5o4)cc3)ccc2-c2ncc(-c3ccc(-c4nc5ccccc5o4)cc3)nc21] |
| 195 | 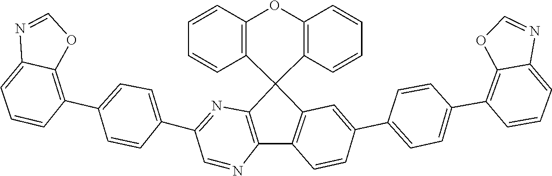 [SMILES: c1ccc2c(c1)Oc1ccccc1C21c2cc(-c3ccc(-c4cccc5ncoc45)cc3)ccc2-c2ncc(-c3ccc(-c4cccc5ncoc45)cc3)nc21] |
| 196 | 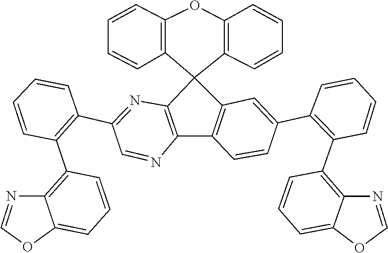 [SMILES: c1ccc2c(c1)Oc1ccccc1C21c2cc(-c3ccccc3-c3cccc4ocnc34)ccc2-c2ncc(-c3ccccc3-c3cccc4ocnc34)nc21] |
| 197 | 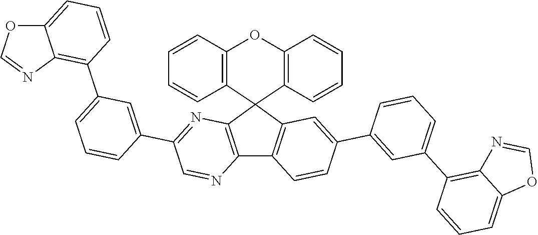 [SMILES: c1cc(-c2ccc3c(c2)C2(c4ccccc4Oc4ccccc42)c2nc(-c4cccc(-c5cccc6ocnc56)c4)cnc2-3)cc(-c2cccc3ocnc23)c1] |
| 198 | 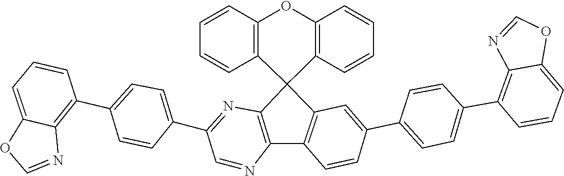 [SMILES: c1ccc2c(c1)Oc1ccccc1C21c2cc(-c3ccc(-c4cccc5ocnc45)cc3)ccc2-c2ncc(-c3ccc(-c4cccc5ocnc45)cc3)nc21] |
| 199 | 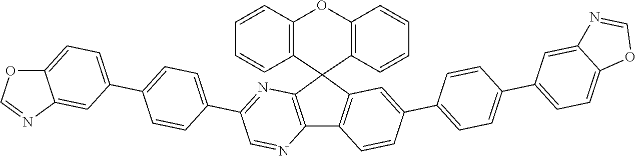 [SMILES: c1ccc2c(c1)Oc1ccccc1C21c2cc(-c3ccc(-c4ccc5ocnc5c4)cc3)ccc2-c2ncc(-c3ccc(-c4ccc5ocnc5c4)cc3)nc21] |
| 200 | 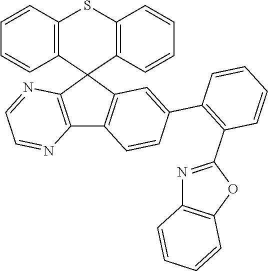 [SMILES: c1ccc2c(c1)Sc1ccccc1C21c2cc(-c3ccccc3-c3nc4ccccc4o3)ccc2-c2nccnc21] |
| 201 | 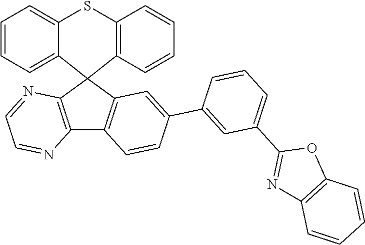 [SMILES: c1cc(-c2ccc3c(c2)C2(c4ccccc4Sc4ccccc42)c2nccnc2-3)cc(-c2nc3ccccc3o2)c1] |
| 202 | 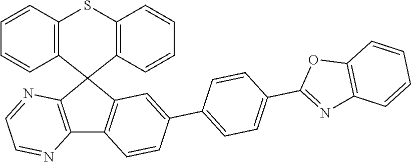 [SMILES: c1ccc2c(c1)Sc1ccccc1C21c2cc(-c3ccc(-c4nc5ccccc5o4)cc3)ccc2-c2nccnc21] |
| 203 | 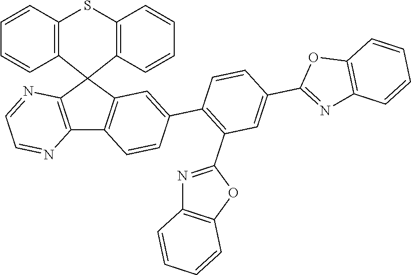 [SMILES: c1ccc2c(c1)Sc1ccccc1C21c2cc(-c3ccc(-c4nc5ccccc5o4)cc3-c3nc4ccccc4o3)ccc2-c2nccnc21] |
| 204 | 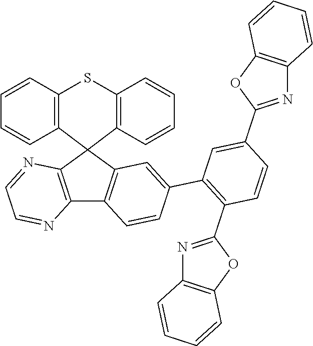 [SMILES: c1ccc2c(c1)Sc1ccccc1C21c2cc(-c3cc(-c4nc5ccccc5o4)ccc3-c3nc4ccccc4o3)ccc2-c2nccnc21] |
| 205 | 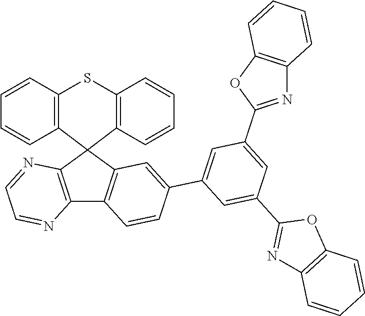 [SMILES: c1ccc2c(c1)Sc1ccccc1C21c2cc(-c3cc(-c4nc5ccccc5o4)cc(-c4nc5ccccc5o4)c3)ccc2-c2nccnc21] |
| 206 | 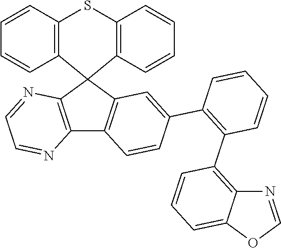 [SMILES: c1ccc2c(c1)Sc1ccccc1C21c2cc(-c3ccccc3-c3cccc4ocnc34)ccc2-c2nccnc21] |
| 207 | 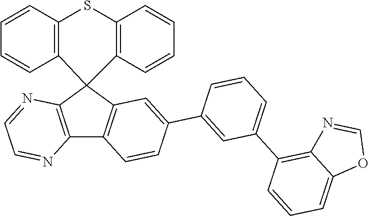 [SMILES: c1cc(-c2ccc3c(c2)C2(c4ccccc4Sc4ccccc42)c2nccnc2-3)cc(-c2cccc3ocnc23)c1] |
| 208 | 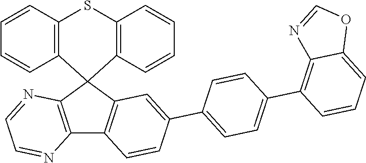 [SMILES: c1ccc2c(c1)Sc1ccccc1C21c2cc(-c3ccc(-c4cccc5ocnc45)cc3)ccc2-c2nccnc21] |
| 209 | 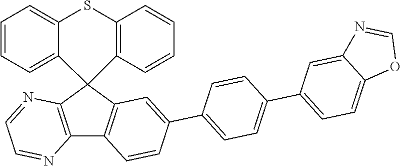 [SMILES: c1ccc2c(c1)Sc1ccccc1C21c2cc(-c3ccc(-c4ccc5ocnc5c4)cc3)ccc2-c2nccnc21] |
| 210 | 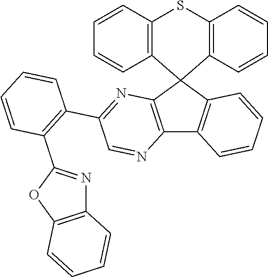 [SMILES: c1ccc2c(c1)Sc1ccccc1C21c2ccccc2-c2ncc(-c3ccccc3-c3nc4ccccc4o3)nc21] |
| 211 | 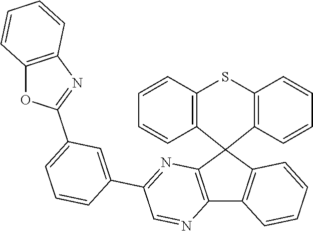 [SMILES: c1cc(-c2cnc3c(n2)C2(c4ccccc4Sc4ccccc42)c2ccccc2-3)cc(-c2nc3ccccc3o2)c1] |
| 212 | 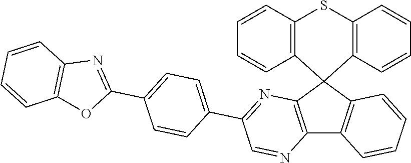 [SMILES: c1ccc2c(c1)Sc1ccccc1C21c2ccccc2-c2ncc(-c3ccc(-c4nc5ccccc5o4)cc3)nc21] |
| 213 | 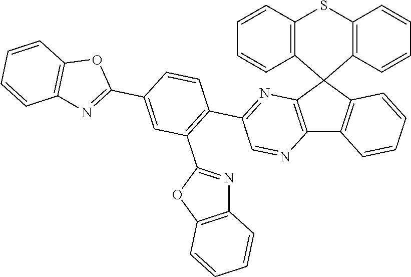 [SMILES: c1ccc2c(c1)Sc1ccccc1C21c2ccccc2-c2ncc(-c3ccc(-c4nc5ccccc5o4)cc3-c3nc4ccccc4o3)nc21] |
| 214 | 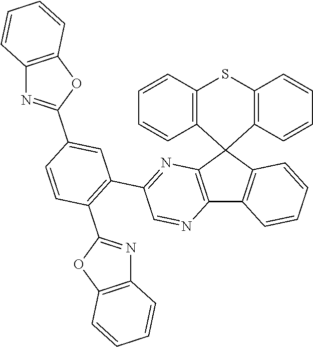 [SMILES: c1ccc2c(c1)Sc1ccccc1C21c2ccccc2-c2ncc(-c3cc(-c4nc5ccccc5o4)ccc3-c3nc4ccccc4o3)nc21] |
| 215 | 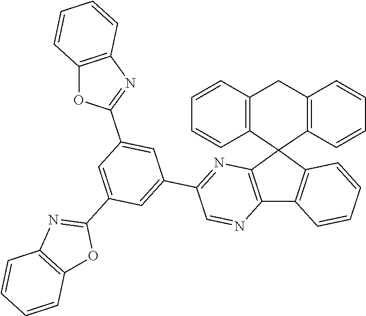 [SMILES: c1ccc2c(c1)Cc1ccccc1C21c2ccccc2-c2ncc(-c3cc(-c4nc5ccccc5o4)cc(-c4nc5ccccc5o4)c3)nc21] |
| 216 | 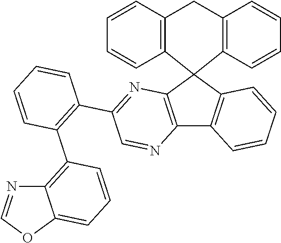 [SMILES: c1ccc2c(c1)Cc1ccccc1C21c2ccccc2-c2ncc(-c3ccccc3-c3cccc4ocnc34)nc21] |
| 217 | 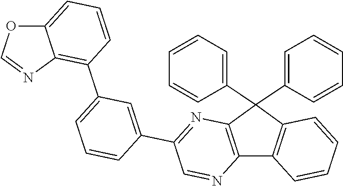 [SMILES: c1ccc(C2(c3ccccc3)c3ccccc3-c3ncc(-c4cccc(-c5cccc6ocnc56)c4)nc32)cc1] |
| 218 | 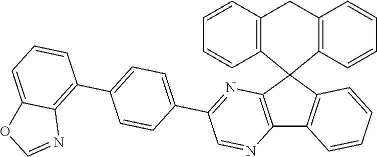 [SMILES: c1ccc2c(c1)Cc1ccccc1C21c2ccccc2-c2ncc(-c3ccc(-c4cccc5ocnc45)cc3)nc21] |
| 219 | 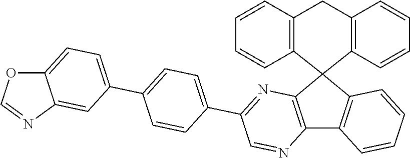 [SMILES: c1ccc2c(c1)Cc1ccccc1C21c2ccccc2-c2ncc(-c3ccc(-c4ccc5ocnc5c4)cc3)nc21] |
| 220 | 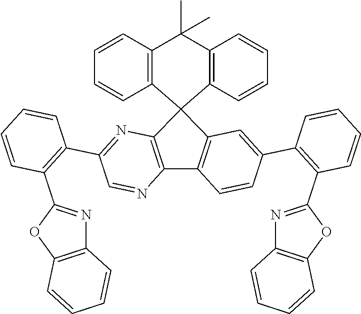 [SMILES: CC1(C)c2ccccc2C2(c3cc(-c4ccccc4-c4nc5ccccc5o4)ccc3-c3ncc(-c4ccccc4-c4nc5ccccc5o4)nc32)c2ccccc21] |
| 221 | 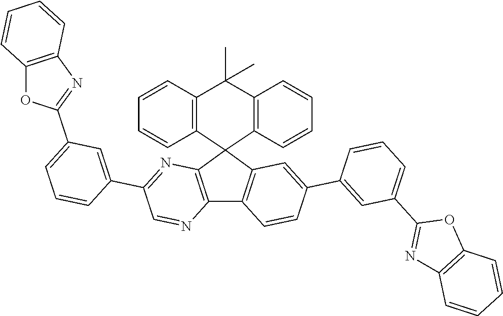 [SMILES: CC1(C)c2ccccc2C2(c3cc(-c4cccc(-c5nc6ccccc6o5)c4)ccc3-c3ncc(-c4cccc(-c5nc6ccccc6o5)c4)nc32)c2ccccc21] |
| 222 | 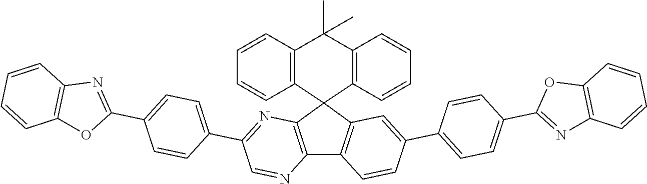 [SMILES: CC1(C)c2ccccc2C2(c3cc(-c4ccc(-c5nc6ccccc6o5)cc4)ccc3-c3ncc(-c4ccc(-c5nc6ccccc6o5)cc4)nc32)c2ccccc21] |
| 223 | 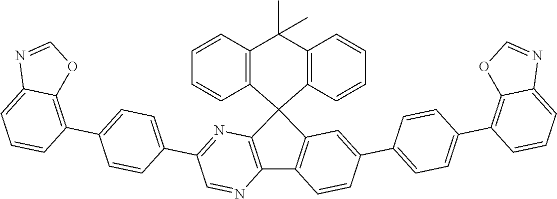 [SMILES: CC1(C)c2ccccc2C2(c3cc(-c4ccc(-c5cccc6ncoc56)cc4)ccc3-c3ncc(-c4ccc(-c5cccc6ncoc56)cc4)nc32)c2ccccc21] |
| 224 | 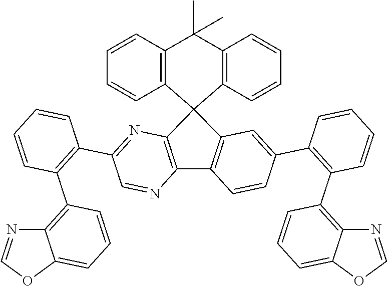 [SMILES: CC1(C)c2ccccc2C2(c3cc(-c4ccccc4-c4cccc5ocnc45)ccc3-c3ncc(-c4ccccc4-c4cccc5ocnc45)nc32)c2ccccc21] |
| 225 | 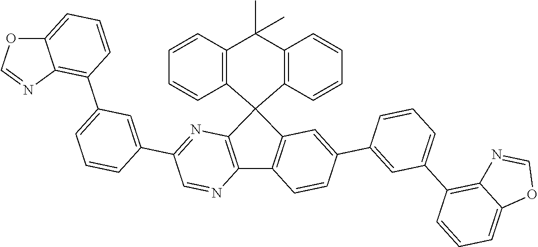 [SMILES: CC1(C)c2ccccc2C2(c3cc(-c4cccc(-c5cccc6ocnc56)c4)ccc3-c3ncc(-c4cccc(-c5cccc6ocnc56)c4)nc32)c2ccccc21] |
| 226 | 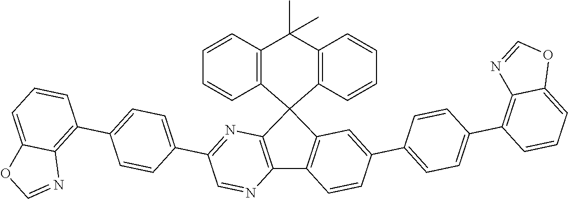 [SMILES: CC1(C)c2ccccc2C2(c3cc(-c4ccc(-c5cccc6ocnc56)cc4)ccc3-c3ncc(-c4ccc(-c5cccc6ocnc56)cc4)nc32)c2ccccc21] |
| 227 | 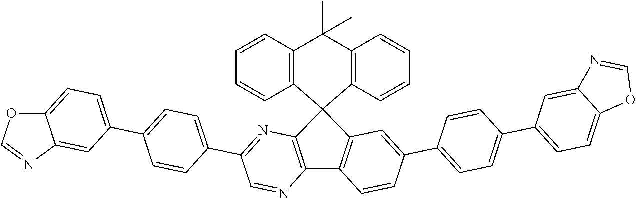 [SMILES: CC1(C)c2ccccc2C2(c3cc(-c4ccc(-c5ccc6ocnc6c5)cc4)ccc3-c3ncc(-c4ccc(-c5ccc6ocnc6c5)cc4)nc32)c2ccccc21] |
| 228 | 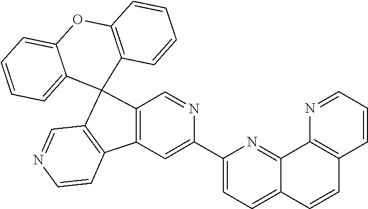 [SMILES: c1ccc2c(c1)Oc1ccccc1C21c2cnccc2-c2cc(-c3ccc4ccc5cccnc5c4n3)ncc21] |
| 229 | 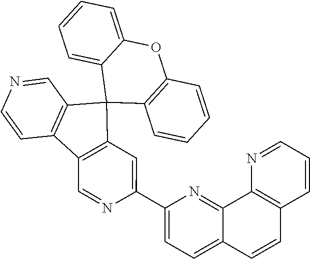 [SMILES: c1ccc2c(c1)Oc1ccccc1C21c2cnccc2-c2cnc(-c3ccc4ccc5cccnc5c4n3)cc21] |
| 230 | 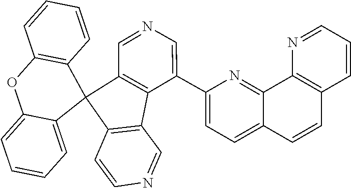 [SMILES: c1ccc2c(c1)Oc1ccccc1C21c2ccncc2-c2c(-c3ccc4ccc5cccnc5c4n3)cncc21] |
| 231 | 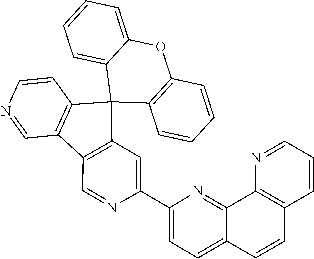 [SMILES: c1ccc2c(c1)Oc1ccccc1C21c2ccncc2-c2cnc(-c3ccc4ccc5cccnc5c4n3)cc21] |
| 232 | 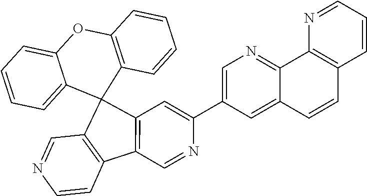 [SMILES: c1ccc2c(c1)Oc1ccccc1C21c2cnccc2-c2cnc(-c3cnc4c(ccc5cccnc54)c3)cc21] |
| 233 | 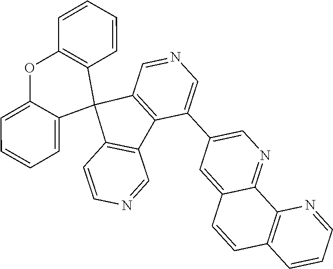 [SMILES: c1ccc2c(c1)Oc1ccccc1C21c2ccncc2-c2c(-c3cnc4c(ccc5cccnc54)c3)cncc21] |
| 234 | 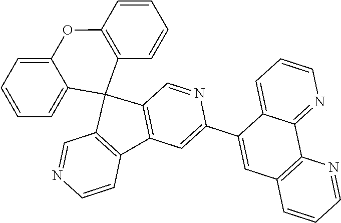 [SMILES: c1ccc2c(c1)Oc1ccccc1C21c2cnccc2-c2cc(-c3cc4cccnc4c4ncccc34)ncc21] |
| 235 | 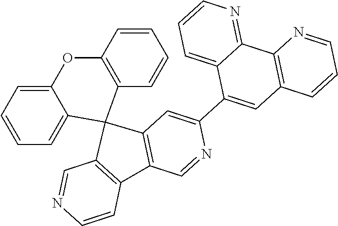 [SMILES: c1ccc2c(c1)Oc1ccccc1C21c2cnccc2-c2cnc(-c3cc4cccnc4c4ncccc34)cc21] |
| 236 | 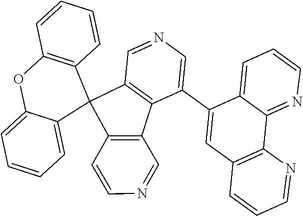 [SMILES: c1ccc2c(c1)Oc1ccccc1C21c2ccncc2-c2c(-c3cc4cccnc4c4ncccc34)cncc21] |
| 237 | 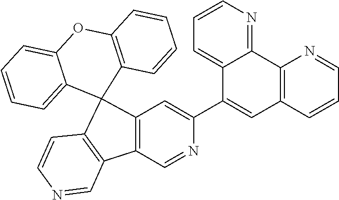 [SMILES: c1ccc2c(c1)Oc1ccccc1C21c2ccncc2-c2cnc(-c3cc4cccnc4c4ncccc34)cc21] |
| 238 | 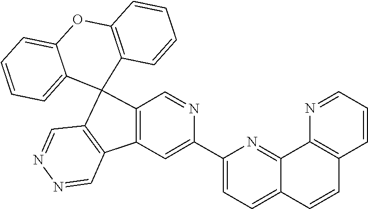 [SMILES: c1ccc2c(c1)Oc1ccccc1C21c2cnncc2-c2cc(-c3ccc4ccc5cccnc5c4n3)ncc21] |
| 239 | 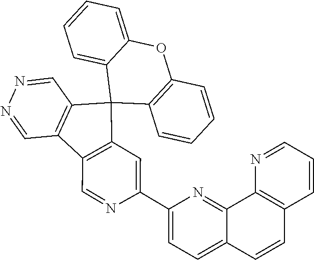 [SMILES: c1ccc2c(c1)Oc1ccccc1C21c2cnncc2-c2cnc(-c3ccc4ccc5cccnc5c4n3)cc21] |
| 240 | 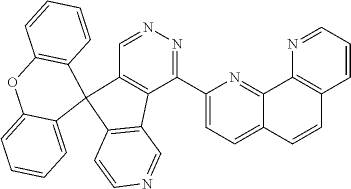 [SMILES: c1ccc2c(c1)Oc1ccccc1C21c2ccncc2-c2c1cnnc2-c1ccc2ccc3cccnc3c2n1] |
| 241 | 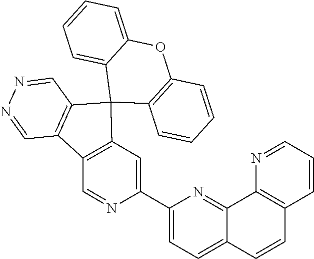 [SMILES: c1ccc2c(c1)Oc1ccccc1C21c2cnncc2-c2cnc(-c3ccc4ccc5cccnc5c4n3)cc21] |
| 242 | 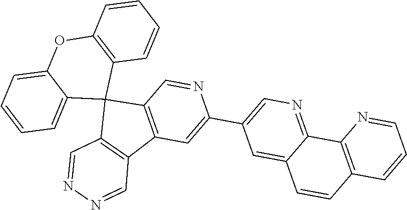 [SMILES: c1ccc2c(c1)Oc1ccccc1C21c2cnncc2-c2cc(-c3cnc4c(ccc5cccnc54)c3)ncc21] |
| 243 | 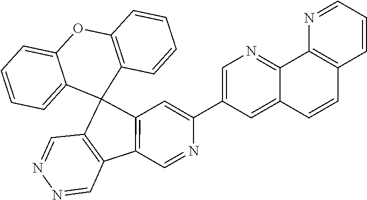 [SMILES: c1ccc2c(c1)Oc1ccccc1C21c2cnncc2-c2cnc(-c3cnc4c(ccc5cccnc54)c3)cc21] |
| 244 | 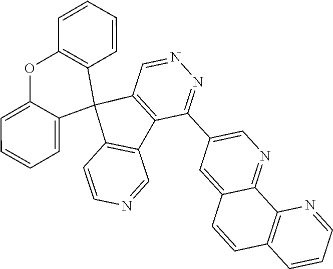 [SMILES: c1ccc2c(c1)Oc1ccccc1C21c2ccncc2-c2c1cnnc2-c1cnc2c(ccc3cccnc32)c1] |
| 245 | 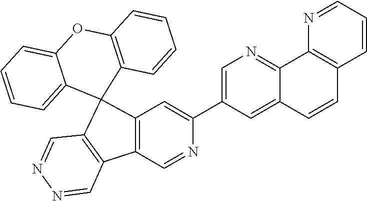 [SMILES: c1ccc2c(c1)Oc1ccccc1C21c2cnncc2-c2cnc(-c3cnc4c(ccc5cccnc54)c3)cc21] |
| 246 | 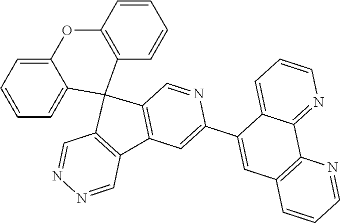 [SMILES: c1ccc2c(c1)Oc1ccccc1C21c2cnncc2-c2cc(-c3cc4cccnc4c4ncccc34)ncc21] |
| 247 | 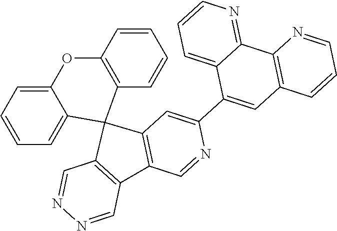 [SMILES: c1ccc2c(c1)Oc1ccccc1C21c2cnncc2-c2cnc(-c3cc4cccnc4c4ncccc34)cc21] |
| 248 | 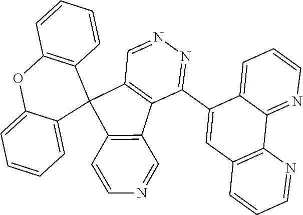 [SMILES: c1ccc2c(c1)Oc1ccccc1C21c2ccncc2-c2c1cnnc2-c1cc2cccnc2c2ncccc12] |
| 249 | 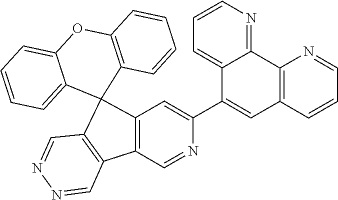 [SMILES: c1ccc2c(c1)Oc1ccccc1C21c2cnncc2-c2cnc(-c3cc4cccnc4c4ncccc34)cc21] |
| 250 | 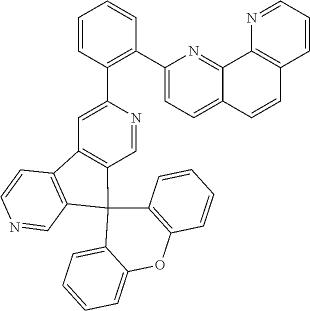 [SMILES: c1ccc2c(c1)Oc1ccccc1C21c2cnccc2-c2cc(-c3ccccc3-c3ccc4ccc5cccnc5c4n3)ncc21] |
| 251 | 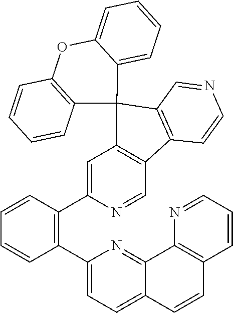 [SMILES: c1ccc2c(c1)Oc1ccccc1C21c2cnccc2-c2cnc(-c3ccccc3-c3ccc4ccc5cccnc5c4n3)cc21] |
| 252 | 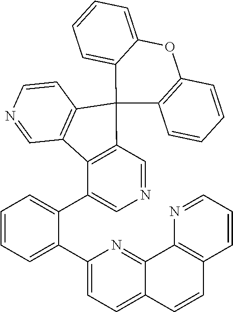 [SMILES: c1ccc2c(c1)Oc1ccccc1C21c2ccncc2-c2c(-c3ccccc3-c3ccc4ccc5cccnc5c4n3)cncc21] |
| 253 | 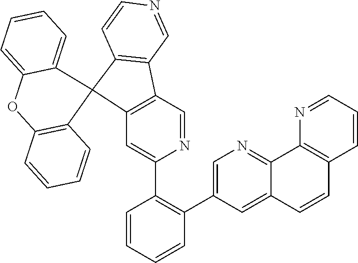 [SMILES: c1ccc2c(c1)Oc1ccccc1C21c2ccncc2-c2cnc(-c3ccccc3-c3cnc4c(ccc5cccnc54)c3)cc21] |
| 254 | 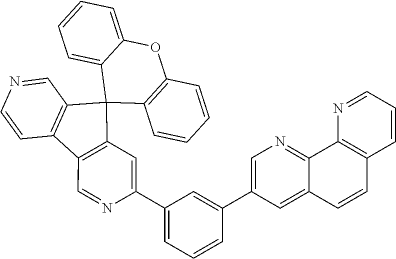 [SMILES: c1cc(-c2cnc3c(ccc4cccnc43)c2)cc(-c2cc3c(cn2)-c2ccncc2C32c3ccccc3Oc3ccccc32)c1] |
| 255 | 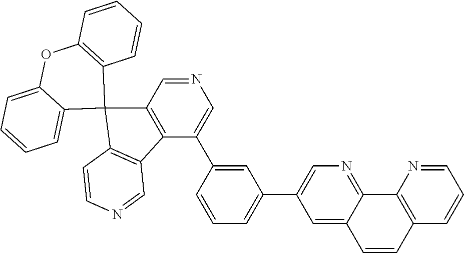 [SMILES: c1cc(-c2cnc3c(ccc4cccnc43)c2)cc(-c2cncc3c2-c2cnccc2C32c3ccccc3Oc3ccccc32)c1] |
| 256 | 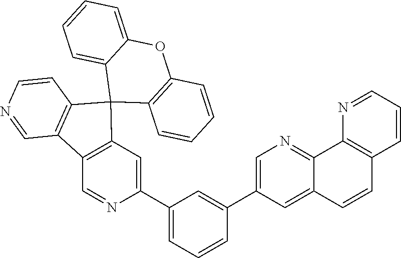 [SMILES: c1cc(-c2cnc3c(ccc4cccnc43)c2)cc(-c2cc3c(cn2)-c2cnccc2C32c3ccccc3Oc3ccccc32)c1] |
| 257 | 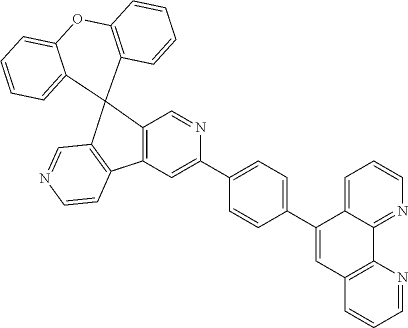 [SMILES: c1ccc2c(c1)Oc1ccccc1C21c2cnccc2-c2cc(-c3ccc(-c4cc5cccnc5c5ncccc45)cc3)ncc21] |
| 258 | 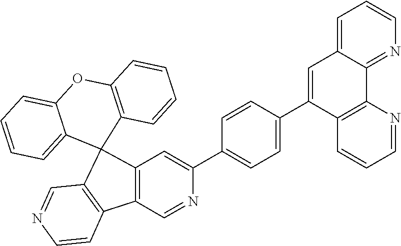 [SMILES: c1ccc2c(c1)Oc1ccccc1C21c2cnccc2-c2cnc(-c3ccc(-c4cc5cccnc5c5ncccc45)cc3)cc21] |
| 259 | 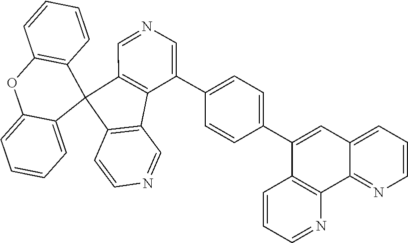 [SMILES: c1ccc2c(c1)Oc1ccccc1C21c2ccncc2-c2c(-c3ccc(-c4cc5cccnc5c5ncccc45)cc3)cncc21] |
| 260 | 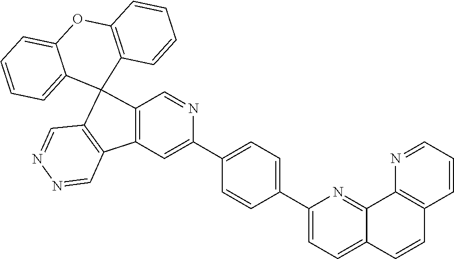 [SMILES: c1ccc2c(c1)Oc1ccccc1C21c2cnncc2-c2cc(-c3ccc(-c4ccc5ccc6cccnc6c5n4)cc3)ncc21] |
| 261 | 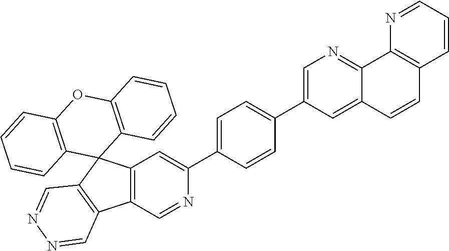 [SMILES: c1ccc2c(c1)Oc1ccccc1C21c2cnncc2-c2cnc(-c3ccc(-c4cnc5c(ccc6cccnc65)c4)cc3)cc21] |
| 262 | 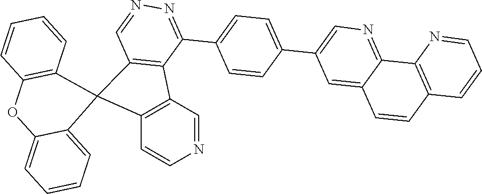 [SMILES: c1ccc2c(c1)Oc1ccccc1C21c2ccncc2-c2c1cnnc2-c1ccc(-c2cnc3c(ccc4cccnc43)c2)cc1] |
| 263 | 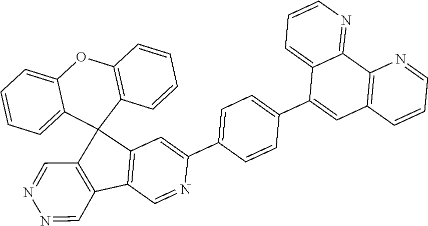 [SMILES: c1ccc2c(c1)Oc1ccccc1C21c2cnncc2-c2cnc(-c3ccc(-c4cc5cccnc5c5ncccc45)cc3)cc21] |
| 264 | 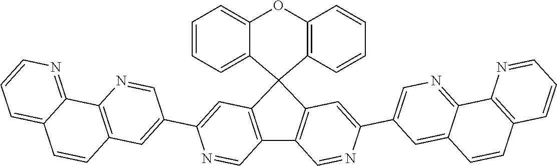 [SMILES: c1ccc2c(c1)Oc1ccccc1C21c2cc(-c3cnc4c(ccc5cccnc54)c3)ncc2-c2cnc(-c3cnc4c(ccc5cccnc54)c3)cc21] |
| 265 | 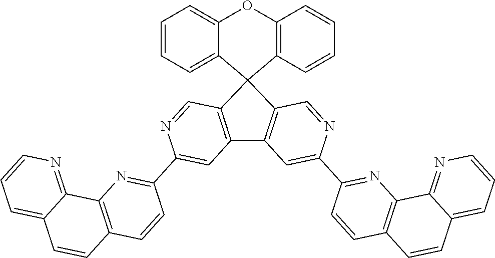 [SMILES: c1ccc2c(c1)Oc1ccccc1C21c2cnc(-c3ccc4ccc5cccnc5c4n3)cc2-c2cc(-c3ccc4ccc5cccnc5c4n3)ncc21] |
| 266 | 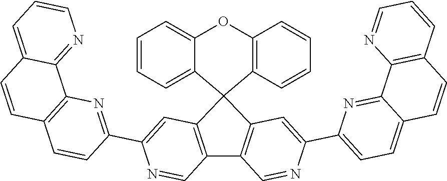 [SMILES: c1ccc2c(c1)Oc1ccccc1C21c2cc(-c3ccc4ccc5cccnc5c4n3)ncc2-c2cnc(-c3ccc4ccc5cccnc5c4n3)cc21] |
| 267 | 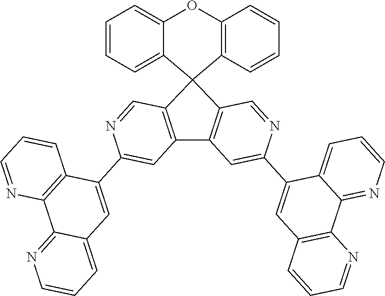 [SMILES: c1ccc2c(c1)Oc1ccccc1C21c2cnc(-c3cc4cccnc4c4ncccc34)cc2-c2cc(-c3cc4cccnc4c4ncccc34)ncc21] |
| 268 | 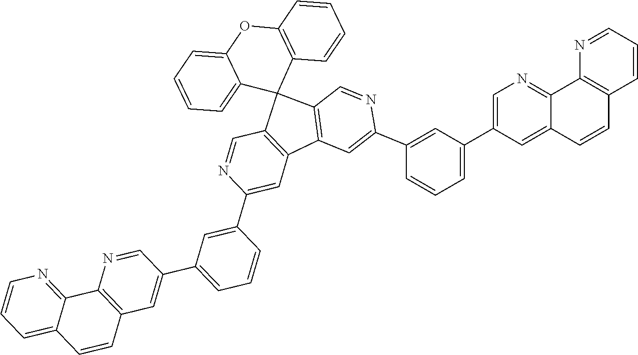 [SMILES: c1cc(-c2cnc3c(ccc4cccnc43)c2)cc(-c2cc3c(cn2)C2(c4ccccc4Oc4ccccc42)c2cnc(-c4cccc(-c5cnc6c(ccc7cccnc76)c5)c4)cc2-3)c1] |
| 269 | 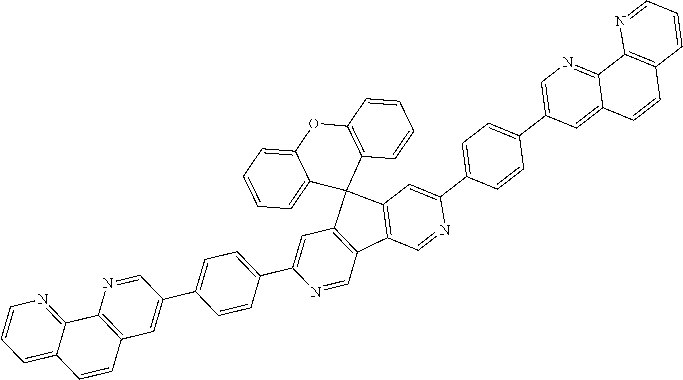 [SMILES: c1ccc2c(c1)Oc1ccccc1C21c2cc(-c3ccc(-c4cnc5c(ccc6cccnc65)c4)cc3)ncc2-c2cnc(-c3ccc(-c4cnc5c(ccc6cccnc65)c4)cc3)cc21] |
| 270 | 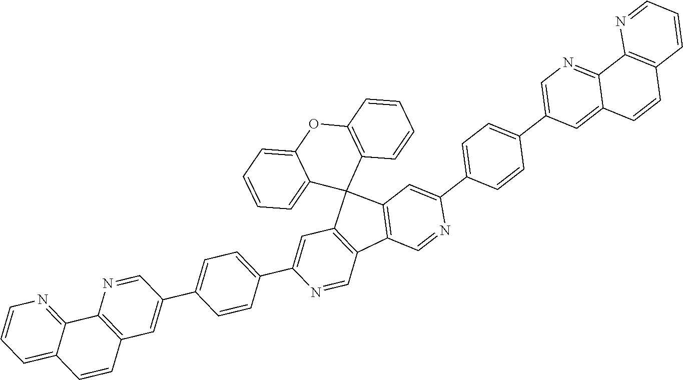 [SMILES: c1ccc2c(c1)Oc1ccccc1C21c2cc(-c3ccc(-c4cnc5c(ccc6cccnc65)c4)cc3)ncc2-c2cnc(-c3ccc(-c4cnc5c(ccc6cccnc65)c4)cc3)cc21] |
| 271 | 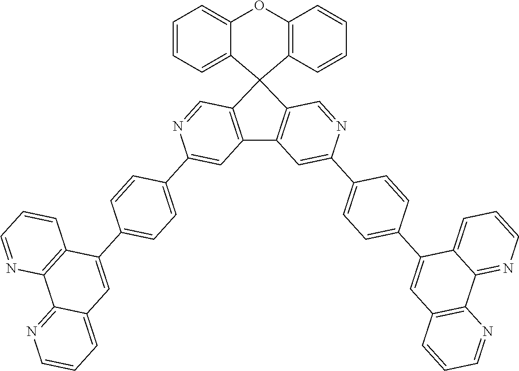 [SMILES: c1ccc2c(c1)Oc1ccccc1C21c2cnc(-c3ccc(-c4cc5cccnc5c5ncccc45)cc3)cc2-c2cc(-c3ccc(-c4cc5cccnc5c5ncccc45)cc3)ncc21] |
| 272 | 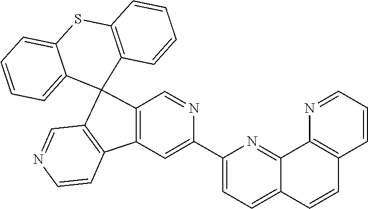 [SMILES: c1ccc2c(c1)Sc1ccccc1C21c2cnccc2-c2cc(-c3ccc4ccc5cccnc5c4n3)ncc21] |
| 273 | 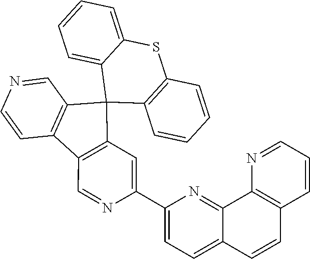 [SMILES: c1ccc2c(c1)Sc1ccccc1C21c2cnccc2-c2cnc(-c3ccc4ccc5cccnc5c4n3)cc21] |
| 274 | 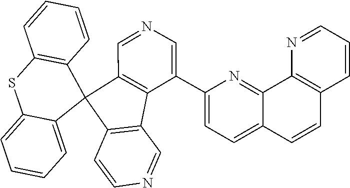 [SMILES: c1ccc2c(c1)Sc1ccccc1C21c2ccncc2-c2c(-c3ccc4ccc5cccnc5c4n3)cncc21] |
| 275 | 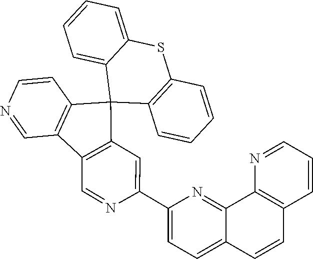 [SMILES: c1ccc2c(c1)Sc1ccccc1C21c2ccncc2-c2cnc(-c3ccc4ccc5cccnc5c4n3)cc21] |
| 276 | 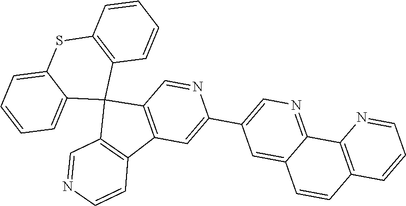 [SMILES: c1ccc2c(c1)Sc1ccccc1C21c2cnccc2-c2cc(-c3cnc4c(ccc5cccnc54)c3)ncc21] |
| 277 | 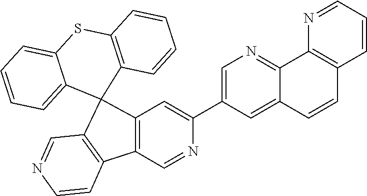 [SMILES: c1ccc2c(c1)Sc1ccccc1C21c2cnccc2-c2cnc(-c3cnc4c(ccc5cccnc54)c3)cc21] |
| 278 | 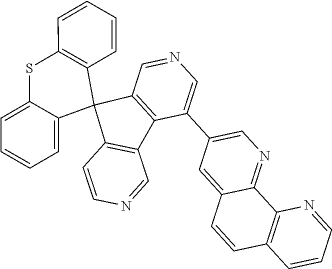 [SMILES: c1ccc2c(c1)Sc1ccccc1C21c2ccncc2-c2c(-c3cnc4c(ccc5cccnc54)c3)cncc21] |
| 279 | 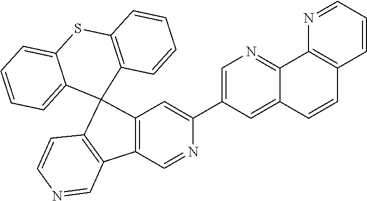 [SMILES: c1ccc2c(c1)Sc1ccccc1C21c2ccncc2-c2cnc(-c3cnc4c(ccc5cccnc54)c3)cc21] |
| 280 | 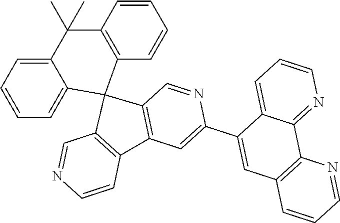 [SMILES: CC1(C)c2ccccc2C2(c3cnccc3-c3cc(-c4cc5cccnc5c5ncccc45)ncc32)c2ccccc21] |
| 281 | 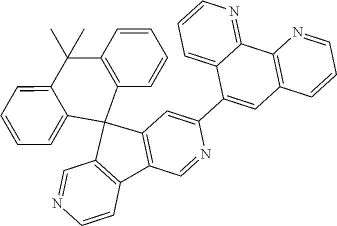 [SMILES: CC1(C)c2ccccc2C2(c3cnccc3-c3cnc(-c4cc5cccnc5c5ncccc45)cc32)c2ccccc21] |
| 282 | 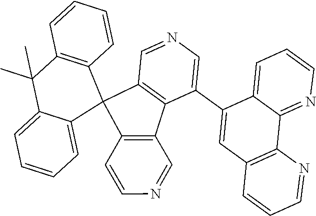 [SMILES: CC1(C)c2ccccc2C2(c3ccncc3-c3c(-c4cc5cccnc5c5ncccc45)cncc32)c2ccccc21] |
| 283 | 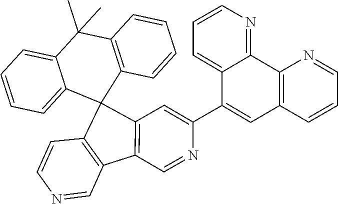 [SMILES: CC1(C)c2ccccc2C2(c3ccncc3-c3cnc(-c4cc5cccnc5c5ncccc45)cc32)c2ccccc21] |
| 284 | 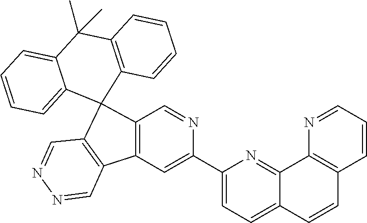 [SMILES: CC1(C)c2ccccc2C2(c3cnncc3-c3cc(-c4ccc5ccc6cccnc6c5n4)ncc32)c2ccccc21] |
| 285 | 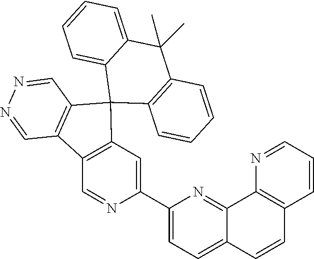 [SMILES: CC1(C)c2ccccc2C2(c3cnncc3-c3cnc(-c4ccc5ccc6cccnc6c5n4)cc32)c2ccccc21] |
| 286 | 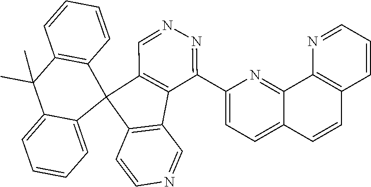 [SMILES: CC1(C)c2ccccc2C2(c3ccncc3-c3c2cnnc3-c2ccc3ccc4cccnc4c3n2)c2ccccc21] |
| 287 | 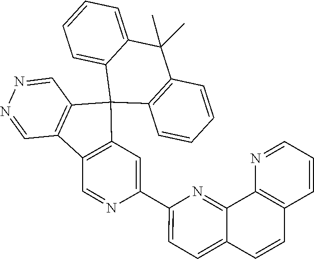 [SMILES: CC1(C)c2ccccc2C2(c3cnncc3-c3cnc(-c4ccc5ccc6cccnc6c5n4)cc32)c2ccccc21] |
| 288 | 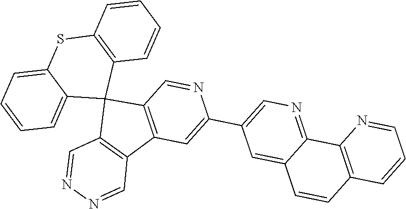 [SMILES: c1ccc2c(c1)Sc1ccccc1C21c2cnncc2-c2cc(-c3cnc4c(ccc5cccnc54)c3)ncc21] |
| 289 | 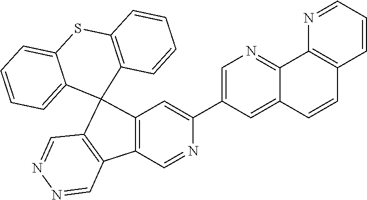 [SMILES: c1ccc2c(c1)Sc1ccccc1C21c2cnncc2-c2cnc(-c3cnc4c(ccc5cccnc54)c3)cc21] |
| 290 | 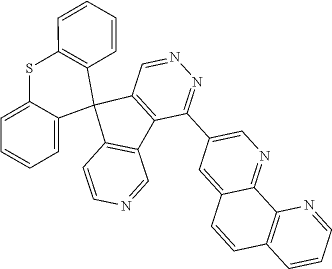 [SMILES: c1ccc2c(c1)Sc1ccccc1C21c2ccncc2-c2c1cnnc2-c1cnc2c(ccc3cccnc32)c1] |
| 291 | 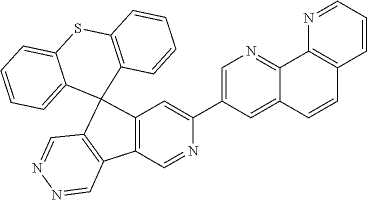 [SMILES: c1ccc2c(c1)Sc1ccccc1C21c2cnncc2-c2cnc(-c3cnc4c(ccc5cccnc54)c3)cc21] |
| 292 | 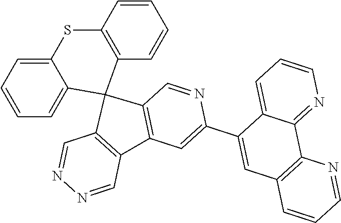 [SMILES: c1ccc2c(c1)Sc1ccccc1C21c2cnncc2-c2cc(-c3cc4cccnc4c4ncccc34)ncc21] |
| 293 | 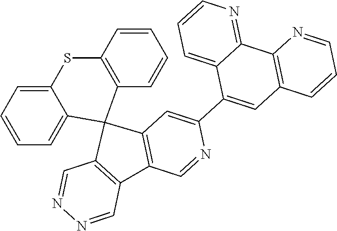 [SMILES: c1ccc2c(c1)Sc1ccccc1C21c2cnncc2-c2cnc(-c3cc4cccnc4c4ncccc34)cc21] |
| 294 | 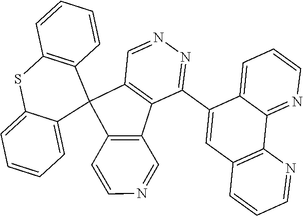 [SMILES: c1ccc2c(c1)Sc1ccccc1C21c2ccncc2-c2c1cnnc2-c1cc2cccnc2c2ncccc12] |
| 295 | 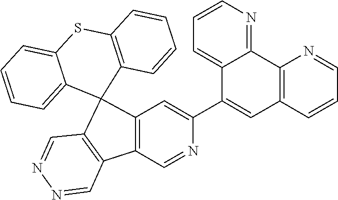 [SMILES: c1ccc2c(c1)Sc1ccccc1C21c2cnncc2-c2cnc(-c3cc4cccnc4c4ncccc34)cc21] |
| 296 | 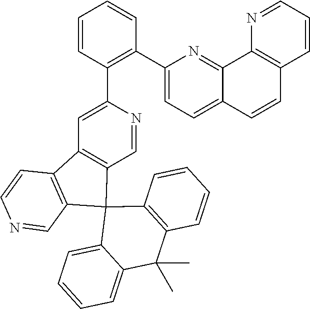 |
| 297 | 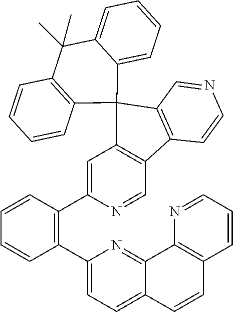 [SMILES: CC1(C)c2ccccc2C2(c3cnccc3-c3cnc(-c4ccccc4-c4ccc5ccc6cccnc6c5n4)cc32)c2ccccc21] |
| 298 | 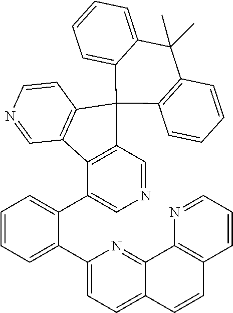 [SMILES: CC1(C)c2ccccc2C2(c3ccncc3-c3c(-c4ccccc4-c4ccc5ccc6cccnc6c5n4)cncc32)c2ccccc21] |
| 299 | 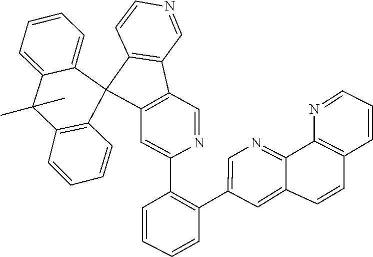 |
| 300 | 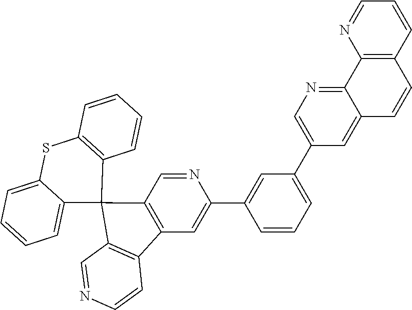 [SMILES: c1cc(-c2cnc3c(ccc4cccnc43)c2)cc(-c2cc3c(cn2)C2(c4ccccc4Sc4ccccc42)c2cnccc2-3)c1] |
| 301 | 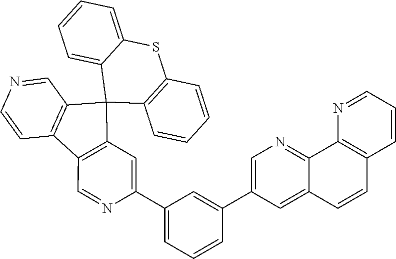 [SMILES: c1cc(-c2cnc3c(ccc4cccnc43)c2)cc(-c2cc3c(cn2)-c2ccncc2C32c3ccccc3Sc3ccccc32)c1] |
| 302 | 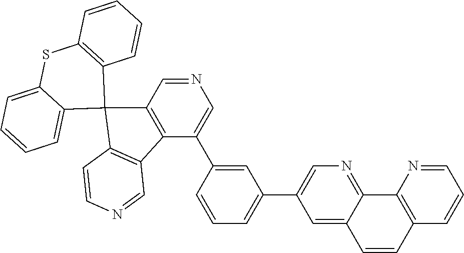 [SMILES: c1cc(-c2cnc3c(ccc4cccnc43)c2)cc(-c2cncc3c2-c2cnccc2C32c3ccccc3Sc3ccccc32)c1] |
| 303 | 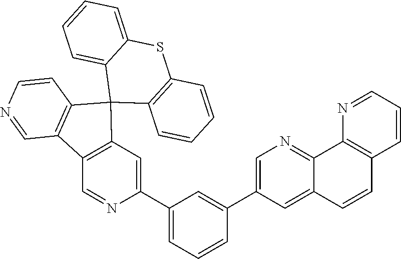 [SMILES: c1cc(-c2cnc3c(ccc4cccnc43)c2)cc(-c2cc3c(cn2)-c2cnccc2C32c3ccccc3Sc3ccccc32)c1] |
| 304 | 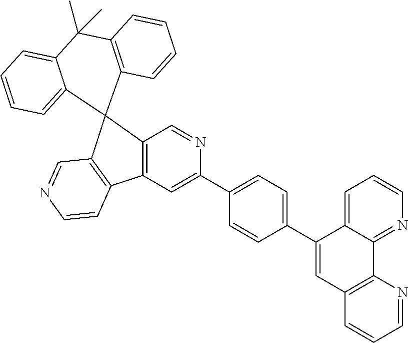 [SMILES: CC1(C)c2ccccc2C2(c3cnccc3-c3cc(-c4ccc(-c5cc6cccnc6c6ncccc56)cc4)ncc32)c2ccccc21] |
| 305 | 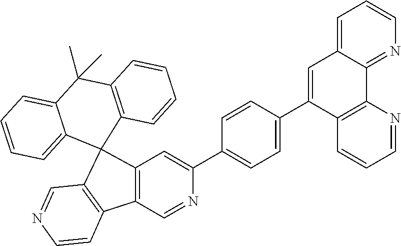 [SMILES: CC1(C)c2ccccc2C2(c3cnccc3-c3cnc(-c4ccc(-c5cc6cccnc6c6ncccc56)cc4)cc32)c2ccccc21] |
| 306 | 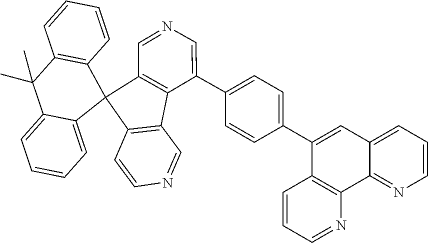 [SMILES: CC1(C)c2ccccc2C2(c3ccncc3-c3c(-c4ccc(-c5cc6cccnc6c6ncccc56)cc4)cncc32)c2ccccc21] |
| 307 | 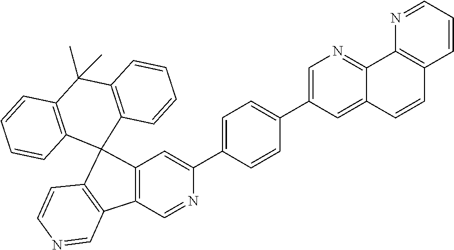 [SMILES: CC1(C)c2ccccc2C2(c3ccncc3-c3cnc(-c4ccc(-c5cnc6c(ccc7cccnc76)c5)cc4)cc32)c2ccccc21] |
| 308 | 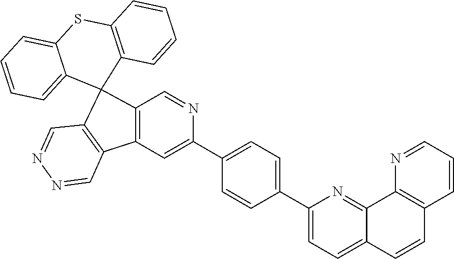 [SMILES: c1ccc2c(c1)Sc1ccccc1C21c2cnncc2-c2cc(-c3ccc(-c4ccc5ccc6cccnc6c5n4)cc3)ncc21] |
| 309 | 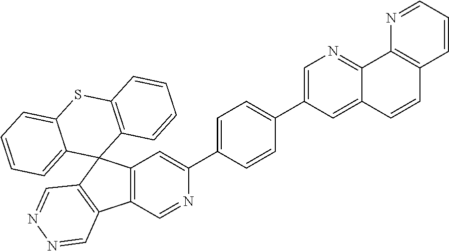 [SMILES: c1ccc2c(c1)Sc1ccccc1C21c2cnncc2-c2cnc(-c3ccc(-c4cnc5c(ccc6cccnc65)c4)cc3)cc21] |
| 310 | 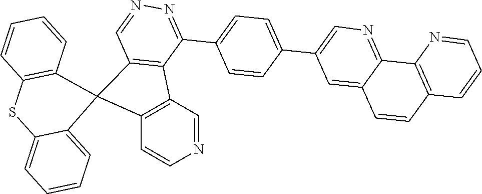 [SMILES: c1ccc2c(c1)Sc1ccccc1C21c2ccncc2-c2c1cnnc2-c1ccc(-c2cnc3c(ccc4cccnc43)c2)cc1] |
| 311 | 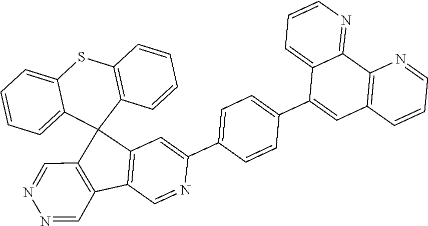 [SMILES: c1ccc2c(c1)Sc1ccccc1C21c2cnncc2-c2cnc(-c3ccc(-c4cc5cccnc5c5ncccc45)cc3)cc21] |
| 312 | 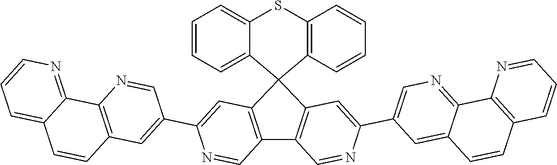 [SMILES: c1ccc2c(c1)Sc1ccccc1C21c2cc(-c3cnc4c(ccc5cccnc54)c3)ncc2-c2cnc(-c3cnc4c(ccc5cccnc54)c3)cc21] |
| 313 | 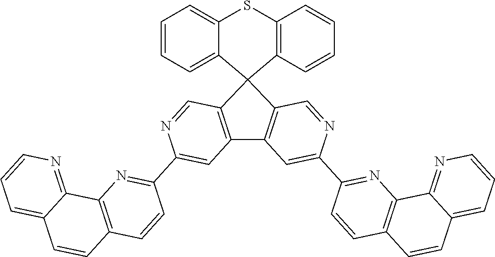 [SMILES: c1ccc2c(c1)Sc1ccccc1C21c2cnc(-c3ccc4ccc5cccnc5c4n3)cc2-c2cc(-c3ccc4ccc5cccnc5c4n3)ncc21] |
| 314 | 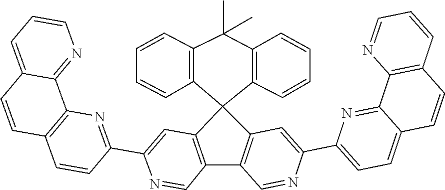 [SMILES: CC1(C)c2ccccc2C2(c3cc(-c4ccc5ccc6cccnc6c5n4)ncc3-c3cnc(-c4ccc5ccc6cccnc6c5n4)cc32)c2ccccc21] |
| 315 | 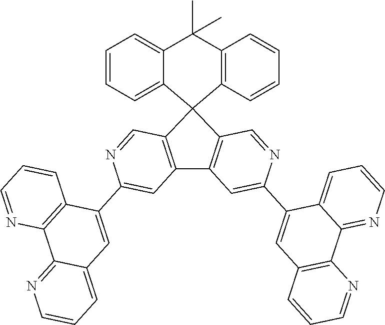 [SMILES: CC1(C)c2ccccc2C2(c3cnc(-c4cc5cccnc5c5ncccc45)cc3-c3cc(-c4cc5cccnc5c5ncccc45)ncc32)c2ccccc21] |
| 316 | 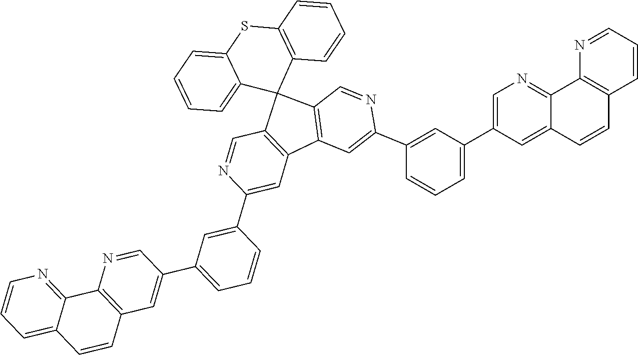 [SMILES: c1cc(-c2cnc3c(ccc4cccnc43)c2)cc(-c2cc3c(cn2)C2(c4ccccc4Sc4ccccc42)c2cnc(-c4cccc(-c5cnc6c(ccc7cccnc76)c5)c4)cc2-3)c1] |
| 317 | 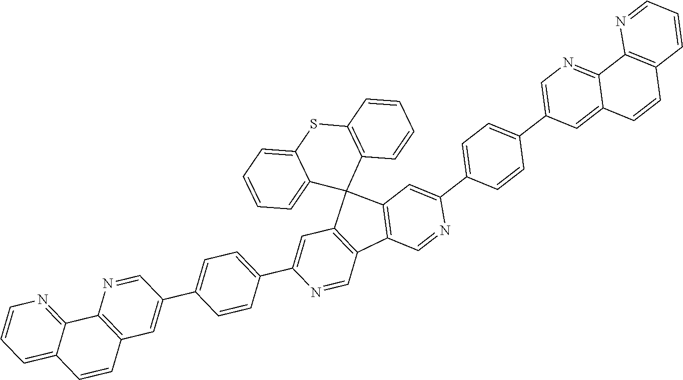 [SMILES: c1ccc2c(c1)Sc1ccccc1C21c2cc(-c3ccc(-c4cnc5c(ccc6cccnc65)c4)cc3)ncc2-c2cnc(-c3ccc(-c4cnc5c(ccc6cccnc65)c4)cc3)cc21] |
| 318 | 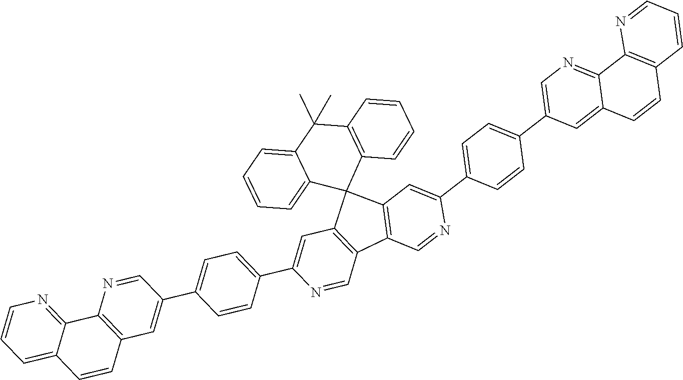 [SMILES: CC1(C)c2ccccc2C2(c3cc(-c4ccc(-c5cnc6c(ccc7cccnc76)c5)cc4)ncc3-c3cnc(-c4ccc(-c5cnc6c(ccc7cccnc76)c5)cc4)cc32)c2ccccc21] |
| 319 | 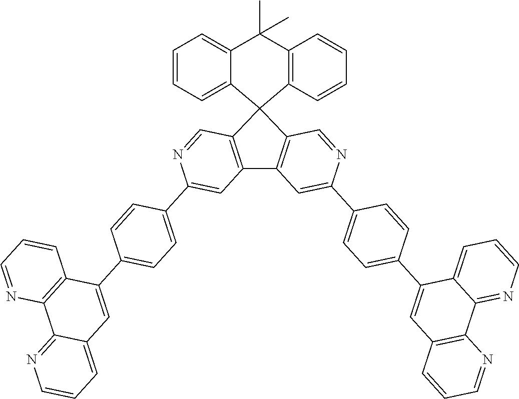 [SMILES: CC1(C)c2ccccc2C2(c3cnc(-c4ccc(-c5cc6cccnc6c6ncccc56)cc4)cc3-c3cc(-c4ccc(-c5cc6cccnc6c6ncccc56)cc4)ncc32)c2ccccc21] |
| 320 | 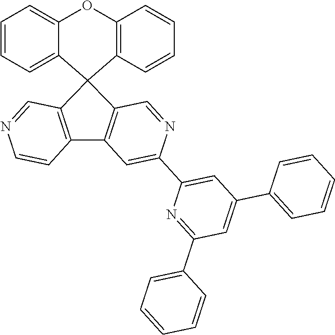 [SMILES: c1ccc(-c2cc(-c3ccccc3)nc(-c3cc4c(cn3)C3(c5ccccc5Oc5ccccc53)c3cnccc3-4)c2)cc1] |
| 321 | 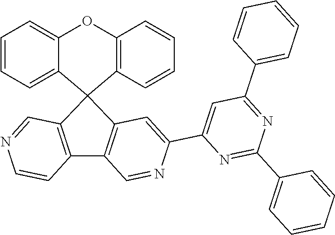 [SMILES: c1ccc(-c2cc(-c3cc4c(cn3)-c3ccncc3C43c4ccccc4Oc4ccccc43)nc(-c3ccccc3)n2)cc1] |
| 322 | 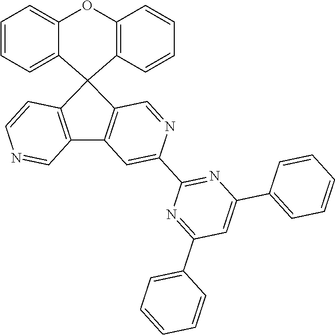 [SMILES: c1ccc(-c2cc(-c3ccccc3)nc(-c3cc4c(cn3)C3(c5ccccc5Oc5ccccc53)c3ccncc3-4)n2)cc1] |
| 323 | 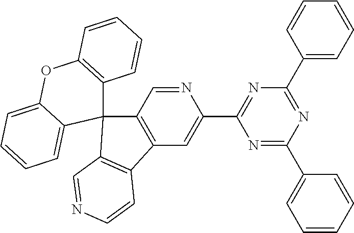 [SMILES: c1ccc(-c2nc(-c3ccccc3)nc(-c3cc4c(cn3)C3(c5ccccc5Oc5ccccc53)c3cnccc3-4)n2)cc1] |
| 324 | 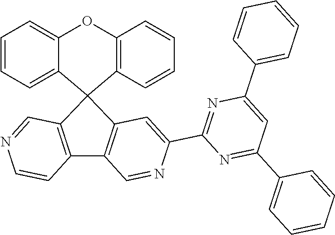 [SMILES: c1ccc(-c2cc(-c3ccccc3)nc(-c3cc4c(cn3)-c3ccncc3C43c4ccccc4Oc4ccccc43)n2)cc1] |
| 325 | 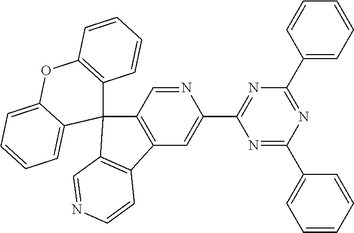 [SMILES: c1ccc(-c2nc(-c3ccccc3)nc(-c3cc4c(cn3)C3(c5ccccc5Oc5ccccc53)c3cnccc3-4)n2)cc1] |
| 326 | 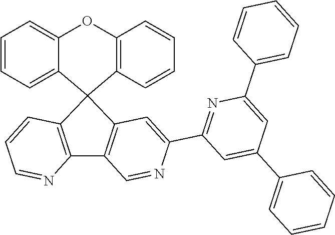 [SMILES: c1ccc(-c2cc(-c3ccccc3)nc(-c3cc4c(cn3)-c3ncccc3C43c4ccccc4Oc4ccccc43)c2)cc1] |
| 327 | 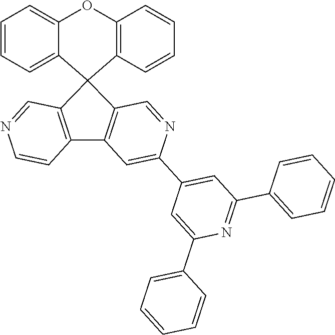 [SMILES: c1ccc(-c2cc(-c3cc4c(cn3)C3(c5ccccc5Oc5ccccc53)c3cnccc3-4)cc(-c3ccccc3)n2)cc1] |
| 328 | 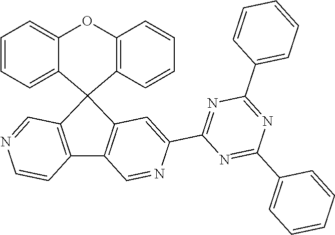 [SMILES: c1ccc(-c2nc(-c3ccccc3)nc(-c3cc4c(cn3)-c3ccncc3C43c4ccccc4Oc4ccccc43)n2)cc1] |
| 329 | 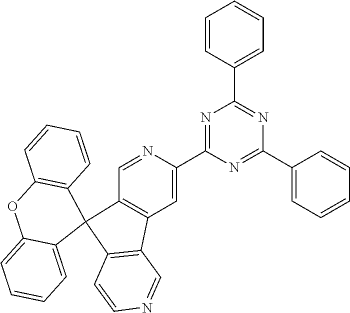 [SMILES: c1ccc(-c2nc(-c3ccccc3)nc(-c3cc4c(cn3)C3(c5ccccc5Oc5ccccc53)c3ccncc3-4)n2)cc1] |
| 330 | 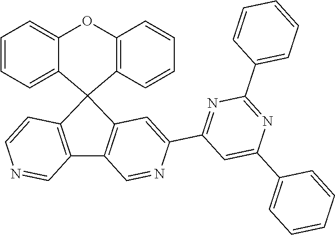 [SMILES: c1ccc(-c2cc(-c3cc4c(cn3)-c3cnccc3C43c4ccccc4Oc4ccccc43)nc(-c3ccccc3)n2)cc1] |
| 331 | 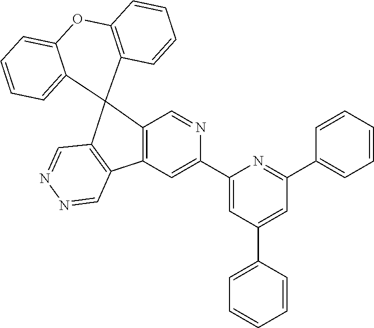 [SMILES: c1ccc(-c2cc(-c3ccccc3)nc(-c3cc4c(cn3)C3(c5ccccc5Oc5ccccc53)c3cnncc3-4)c2)cc1] |
| 332 | 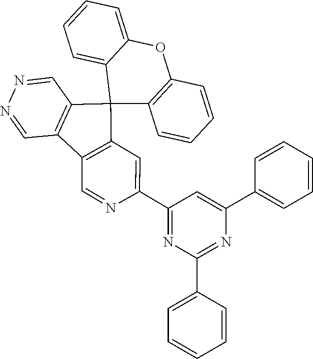 [SMILES: c1ccc(-c2cc(-c3cc4c(cn3)-c3cnncc3C43c4ccccc4Oc4ccccc43)nc(-c3ccccc3)n2)cc1] |
| 333 | 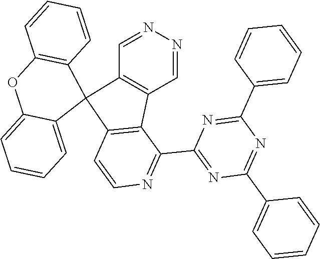 [SMILES: c1ccc(-c2nc(-c3ccccc3)nc(-c3nccc4c3-c3cnncc3C43c4ccccc4Oc4ccccc43)n2)cc1] |
| 334 | 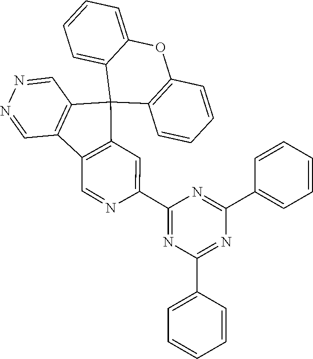 [SMILES: c1ccc(-c2nc(-c3ccccc3)nc(-c3cc4c(cn3)-c3cnncc3C43c4ccccc4Oc4ccccc43)n2)cc1] |
| 335 | 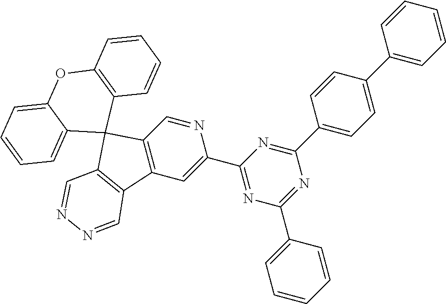 [SMILES: c1ccc(-c2ccc(-c3nc(-c4ccccc4)nc(-c4cc5c(cn4)C4(c6ccccc6Oc6ccccc64)c4cnncc4-5)n3)cc2)cc1] |
| 336 | 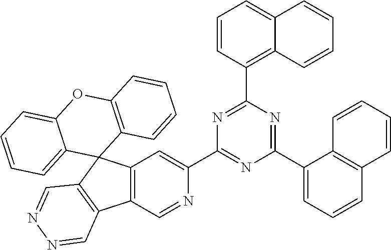 [SMILES: c1ccc2c(c1)Oc1ccccc1C21c2cnncc2-c2cnc(-c3nc(-c4cccc5ccccc45)nc(-c4cccc5ccccc45)n3)cc21] |
| 337 | 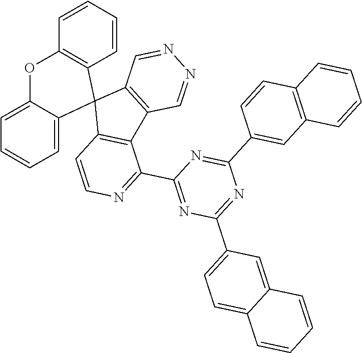 [SMILES: c1ccc2c(c1)Oc1ccccc1C21c2cnncc2-c2c1ccnc2-c1nc(-c2ccc3ccccc3c2)nc(-c2ccc3ccccc3c2)n1] |
| 338 | 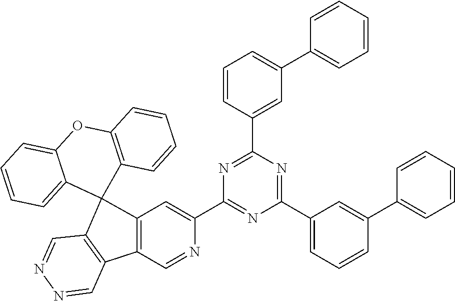 [SMILES: c1ccc(-c2cccc(-c3nc(-c4cccc(-c5ccccc5)c4)nc(-c4cc5c(cn4)-c4cnncc4C54c5ccccc5Oc5ccccc54)n3)c2)cc1] |
| 339 | 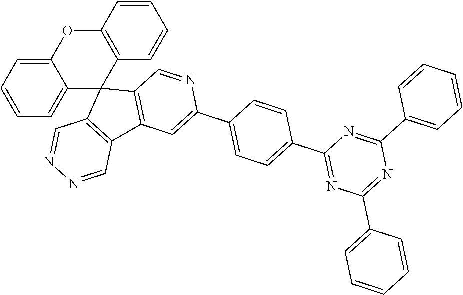 [SMILES: c1ccc(-c2nc(-c3ccccc3)nc(-c3ccc(-c4cc5c(cn4)C4(c6ccccc6Oc6ccccc64)c4cnncc4-5)cc3)n2)cc1] |
| 340 | 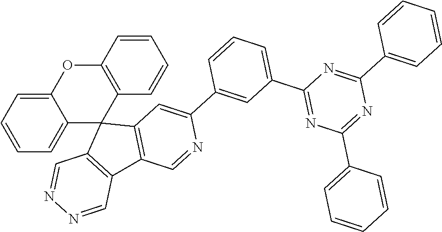 [SMILES: c1ccc(-c2nc(-c3ccccc3)nc(-c3cccc(-c4cc5c(cn4)-c4cnncc4C54c5ccccc5Oc5ccccc54)c3)n2)cc1] |
| 341 | 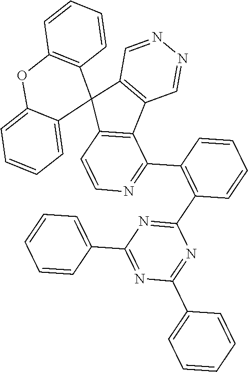 [SMILES: c1ccc(-c2nc(-c3ccccc3)nc(-c3ccccc3-c3nccc4c3-c3cnncc3C43c4ccccc4Oc4ccccc43)n2)cc1] |
| 342 | 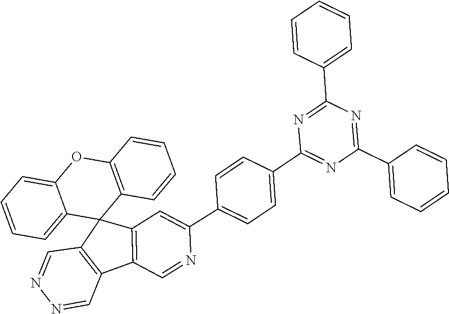 [SMILES: c1ccc(-c2nc(-c3ccccc3)nc(-c3ccc(-c4cc5c(cn4)-c4cnncc4C54c5ccccc5Oc5ccccc54)cc3)n2)cc1] |
| 343 | 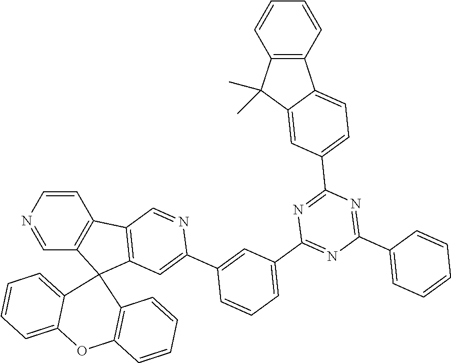 [SMILES: CC1(C)c2ccccc2-c2ccc(-c3nc(-c4ccccc4)nc(-c4cccc(-c5cc6c(cn5)-c5ccncc5C65c6ccccc6Oc6ccccc65)c4)n3)cc21] |
| 344 | 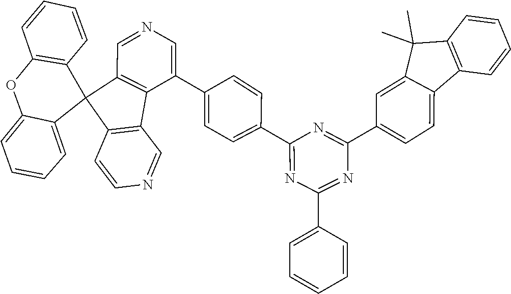 [SMILES: CC1(C)c2ccccc2-c2ccc(-c3nc(-c4ccccc4)nc(-c4ccc(-c5cncc6c5-c5cnccc5C65c6ccccc6Oc6ccccc65)cc4)n3)cc21] |
| 345 | 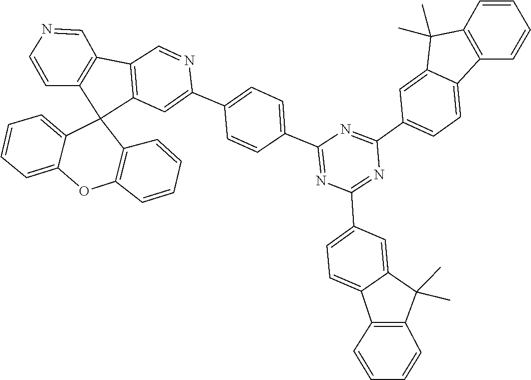 [SMILES: CC1(C)c2ccccc2-c2ccc(-c3nc(-c4ccc(-c5cc6c(cn5)-c5cnccc5C65c6ccccc6Oc6ccccc65)cc4)nc(-c4ccc5c(c4)C(C)(C)c4ccccc4-5)n3)cc21] |
| 346 | 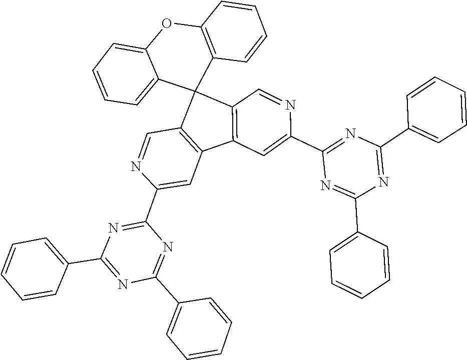 [SMILES: c1ccc(-c2nc(-c3ccccc3)nc(-c3cc4c(cn3)C3(c5ccccc5Oc5ccccc53)c3cnc(-c5nc(-c6ccccc6)nc(-c6ccccc6)n5)cc3-4)n2)cc1] |
| 347 | 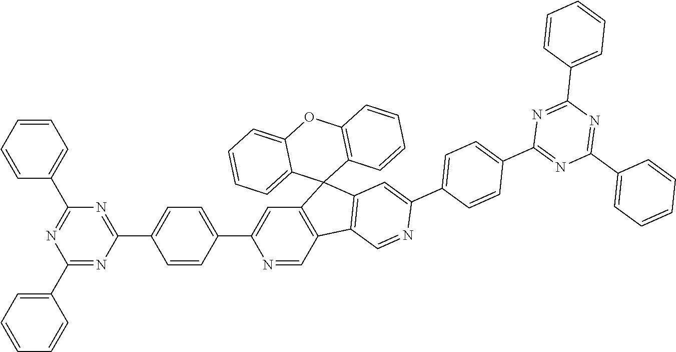 [SMILES: c1ccc(-c2nc(-c3ccccc3)nc(-c3ccc(-c4cc5c(cn4)-c4cnc(-c6ccc(-c7nc(-c8ccccc8)nc(-c8ccccc8)n7)cc6)cc4C54c5ccccc5Oc5ccccc54)cc3)n2)cc1] |
| 348 | 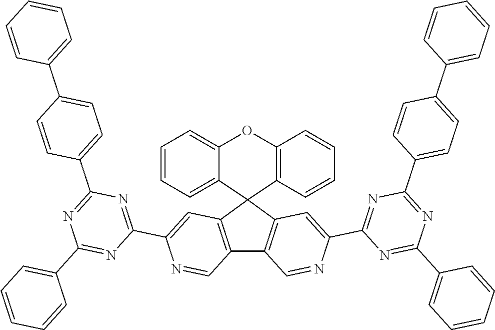 [SMILES: c1ccc(-c2ccc(-c3nc(-c4ccccc4)nc(-c4cc5c(cn4)-c4cnc(-c6nc(-c7ccccc7)nc(-c7ccc(-c8ccccc8)cc7)n6)cc4C54c5ccccc5Oc5ccccc54)n3)cc2)cc1] |
| 349 | 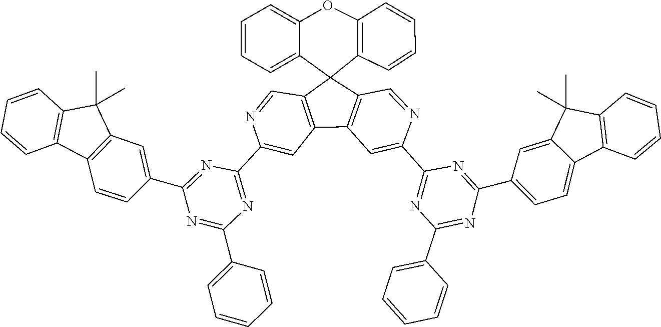 [SMILES: CC1(C)c2ccccc2-c2ccc(-c3nc(-c4ccccc4)nc(-c4cc5c(cn4)C4(c6ccccc6Oc6ccccc64)c4cnc(-c6nc(-c7ccccc7)nc(-c7ccc8c(c7)C(C)(C)c7ccccc7-8)n6)cc4-5)n3)cc21] |
| 350 | 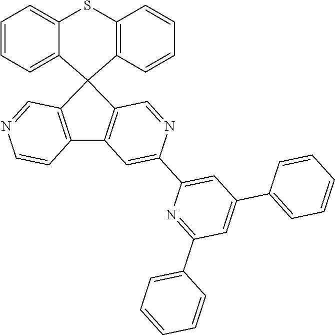 [SMILES: c1ccc(-c2cc(-c3ccccc3)nc(-c3cc4c(cn3)C3(c5ccccc5Sc5ccccc53)c3cnccc3-4)c2)cc1] |
| 351 | 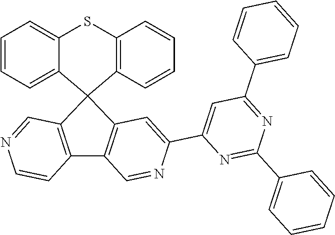 [SMILES: c1ccc(-c2cc(-c3cc4c(cn3)-c3ccncc3C43c4ccccc4Sc4ccccc43)nc(-c3ccccc3)n2)cc1] |
| 352 | 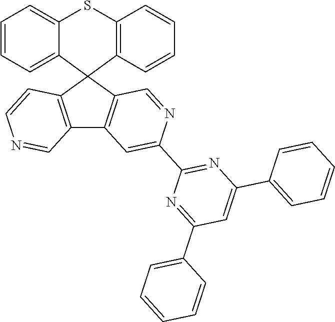 [SMILES: c1ccc(-c2cc(-c3ccccc3)nc(-c3cc4c(cn3)C3(c5ccccc5Sc5ccccc53)c3ccncc3-4)n2)cc1] |
| 353 | 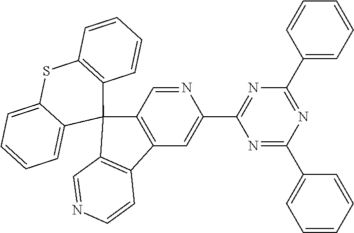 [SMILES: c1ccc(-c2nc(-c3ccccc3)nc(-c3cc4c(cn3)C3(c5ccccc5Sc5ccccc53)c3cnccc3-4)n2)cc1] |
| 354 | 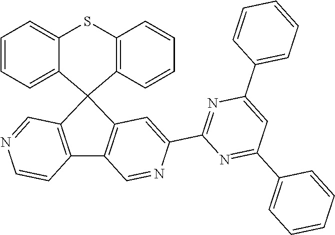 [SMILES: c1ccc(-c2cc(-c3ccccc3)nc(-c3cc4c(cn3)-c3ccncc3C43c4ccccc4Sc4ccccc43)n2)cc1] |
| 355 | 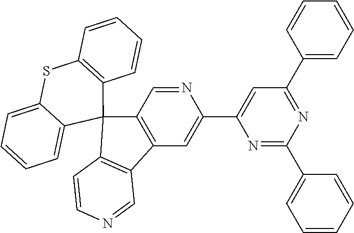 [SMILES: c1ccc(-c2cc(-c3cc4c(cn3)C3(c5ccccc5Sc5ccccc53)c3ccncc3-4)nc(-c3ccccc3)n2)cc1] |
| 356 | 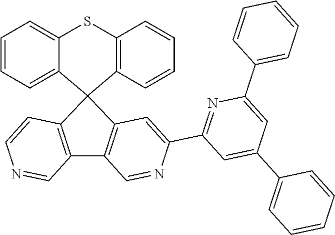 [SMILES: c1ccc(-c2cc(-c3ccccc3)nc(-c3cc4c(cn3)-c3cnccc3C43c4ccccc4Sc4ccccc43)c2)cc1] |
| 357 | 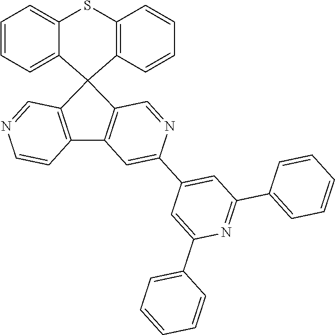 [SMILES: c1ccc(-c2cc(-c3cc4c(cn3)C3(c5ccccc5Sc5ccccc53)c3cnccc3-4)cc(-c3ccccc3)n2)cc1] |
| 358 | 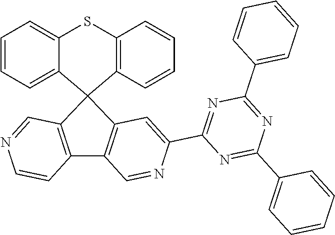 [SMILES: c1ccc(-c2nc(-c3ccccc3)nc(-c3cc4c(cn3)-c3ccncc3C43c4ccccc4Sc4ccccc43)n2)cc1] |
| 359 | 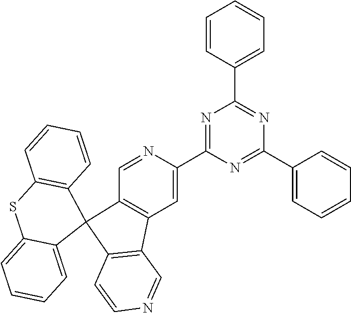 [SMILES: c1ccc(-c2nc(-c3ccccc3)nc(-c3cc4c(cn3)C3(c5ccccc5Sc5ccccc53)c3ccncc3-4)n2)cc1] |
| 360 | 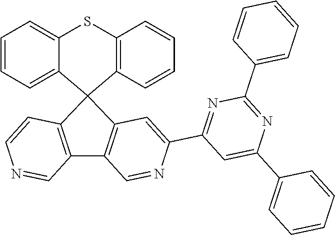 [SMILES: c1ccc(-c2cc(-c3cc4c(cn3)-c3cnccc3C43c4ccccc4Sc4ccccc43)nc(-c3ccccc3)n2)cc1] |
| 361 | 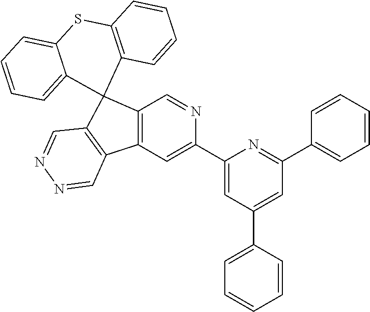 [SMILES: c1ccc(-c2cc(-c3ccccc3)nc(-c3cc4c(cn3)C3(c5ccccc5Sc5ccccc53)c3cnncc3-4)c2)cc1] |
| 362 | 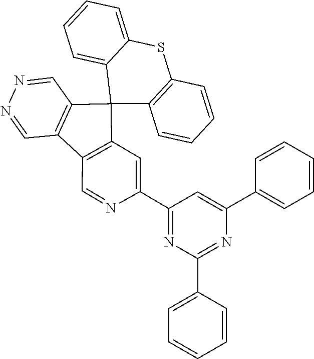 [SMILES: c1ccc(-c2cc(-c3cc4c(cn3)-c3cnncc3C43c4ccccc4Sc4ccccc43)nc(-c3ccccc3)n2)cc1] |
| 363 | 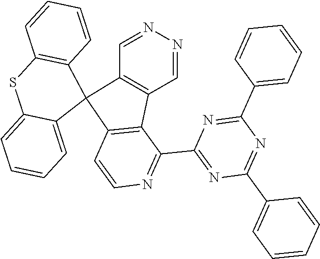 [SMILES: c1ccc(-c2nc(-c3ccccc3)nc(-c3nccc4c3-c3cnncc3C43c4ccccc4Sc4ccccc43)n2)cc1] |
| 364 | 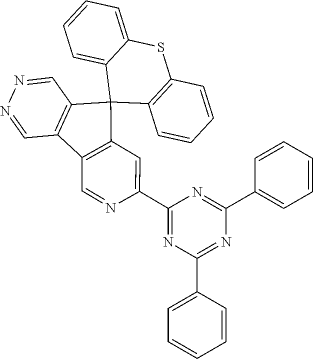 [SMILES: c1ccc(-c2nc(-c3ccccc3)nc(-c3cc4c(cn3)-c3cnncc3C43c4ccccc4Sc4ccccc43)n2)cc1] |
| 365 | 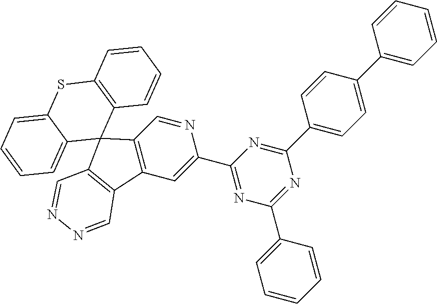 [SMILES: c1ccc(-c2ccc(-c3nc(-c4ccccc4)nc(-c4cc5c(cn4)C4(c6ccccc6Sc6ccccc64)c4cnncc4-5)n3)cc2)cc1] |
| 366 | 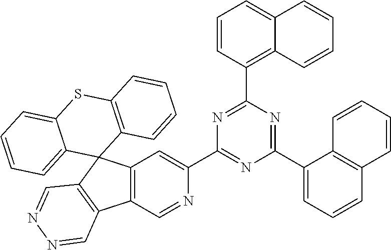 [SMILES: c1ccc2c(c1)Sc1ccccc1C21c2cnncc2-c2cnc(-c3nc(-c4cccc5ccccc45)nc(-c4cccc5ccccc45)n3)cc21] |
| 367 | 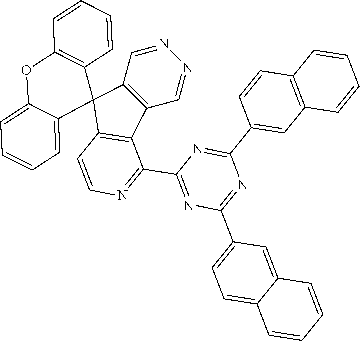 [SMILES: c1ccc2c(c1)Oc1ccccc1C21c2cnncc2-c2c1ccnc2-c1nc(-c2ccc3ccccc3c2)nc(-c2ccc3ccccc3c2)n1] |
| 368 | 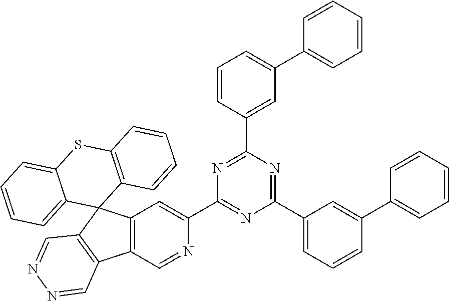 [SMILES: c1ccc(-c2cccc(-c3nc(-c4cccc(-c5ccccc5)c4)nc(-c4cc5c(cn4)-c4cnncc4C54c5ccccc5Sc5ccccc54)n3)c2)cc1] |
| 369 | 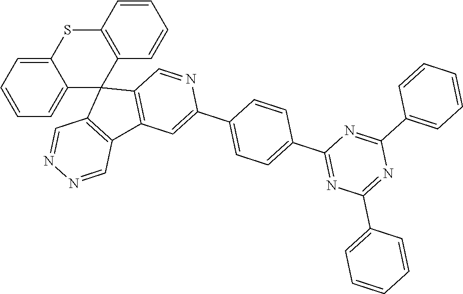 [SMILES: c1ccc(-c2nc(-c3ccccc3)nc(-c3ccc(-c4cc5c(cn4)C4(c6ccccc6Sc6ccccc64)c4cnncc4-5)cc3)n2)cc1] |
| 370 | 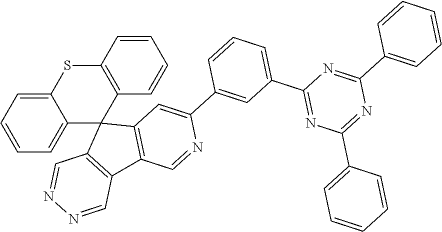 [SMILES: c1ccc(-c2nc(-c3ccccc3)nc(-c3cccc(-c4cc5c(cn4)-c4cnncc4C54c5ccccc5Sc5ccccc54)c3)n2)cc1] |
| 371 | 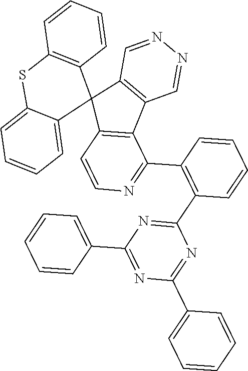 [SMILES: c1ccc(-c2nc(-c3ccccc3)nc(-c3ccccc3-c3nccc4c3-c3cnncc3C43c4ccccc4Sc4ccccc43)n2)cc1] |
| 372 | 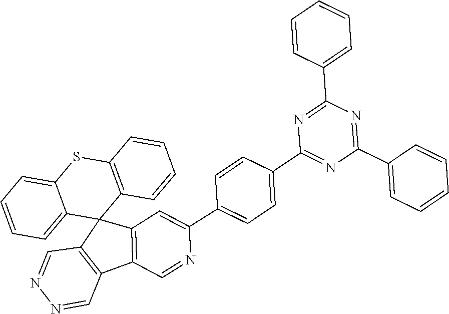 [SMILES: c1ccc(-c2nc(-c3ccccc3)nc(-c3ccc(-c4cc5c(cn4)-c4cnncc4C54c5ccccc5Sc5ccccc54)cc3)n2)cc1] |
| 373 | 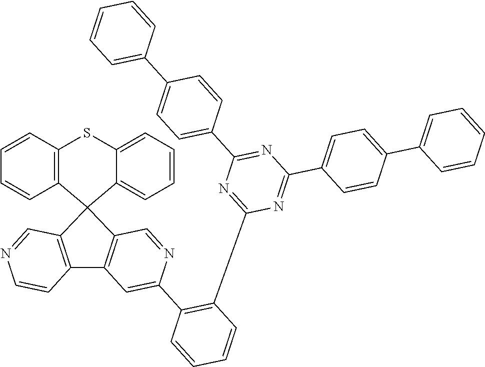 [SMILES: c1ccc(-c2ccc(-c3nc(-c4ccc(-c5ccccc5)cc4)nc(-c4ccccc4-c4cc5c(cn4)C4(c6ccccc6Sc6ccccc64)c4cnccc4-5)n3)cc2)cc1] |
| 374 | 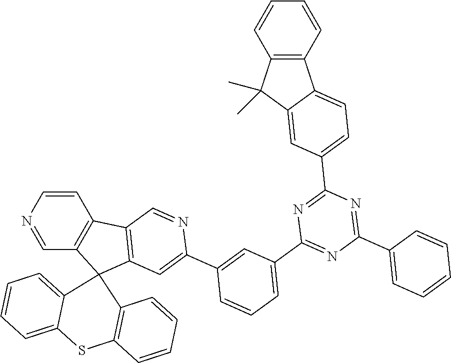 [SMILES: CC1(C)c2ccccc2-c2ccc(-c3nc(-c4ccccc4)nc(-c4cccc(-c5cc6c(cn5)-c5ccncc5C65c6ccccc6Sc6ccccc65)c4)n3)cc21] |
| 375 | 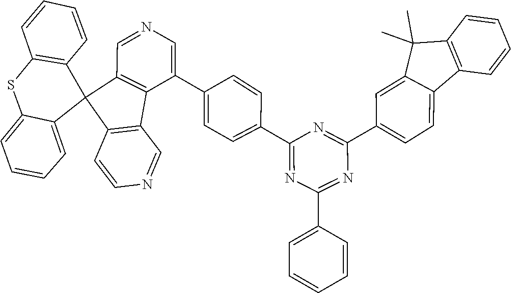 [SMILES: CC1(C)c2ccccc2-c2ccc(-c3nc(-c4ccccc4)nc(-c4ccc(-c5cncc6c5-c5cnccc5C65c6ccccc6Sc6ccccc65)cc4)n3)cc21] |
| 376 | 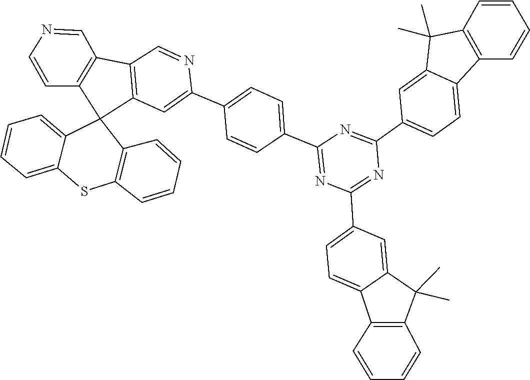 [SMILES: CC1(C)c2ccccc2-c2ccc(-c3nc(-c4ccc(-c5cc6c(cn5)-c5cnccc5C65c6ccccc6Sc6ccccc65)cc4)nc(-c4ccc5c(c4)C(C)(C)c4ccccc4-5)n3)cc21] |
| 377 | 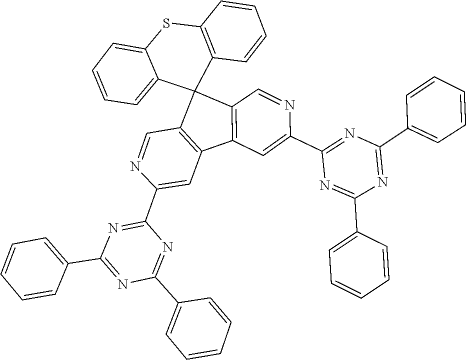 [SMILES: c1ccc(-c2nc(-c3ccccc3)nc(-c3cc4c(cn3)C3(c5ccccc5Sc5ccccc53)c3cnc(-c5nc(-c6ccccc6)nc(-c6ccccc6)n5)cc3-4)n2)cc1] |
| 378 | 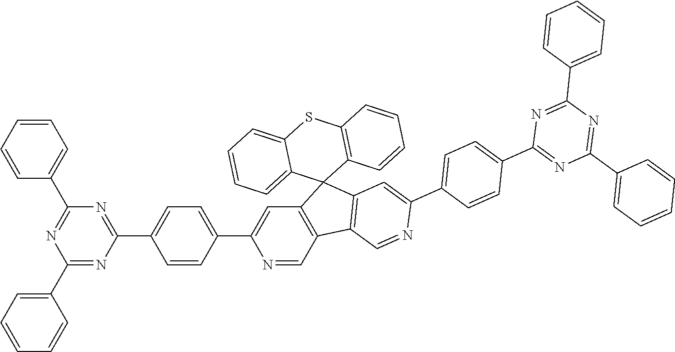 [SMILES: c1ccc(-c2nc(-c3ccccc3)nc(-c3ccc(-c4cc5c(cn4)-c4cnc(-c6ccc(-c7nc(-c8ccccc8)nc(-c8ccccc8)n7)cc6)cc4C54c5ccccc5Sc5ccccc54)cc3)n2)cc1] |
| 379 | 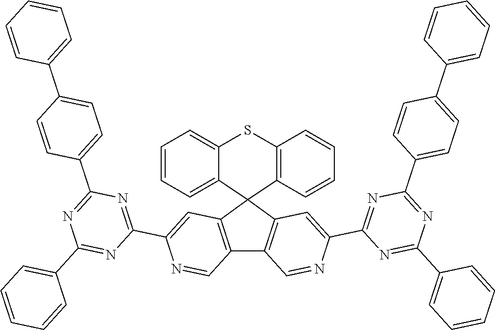 [SMILES: c1ccc(-c2ccc(-c3nc(-c4ccccc4)nc(-c4cc5c(cn4)-c4cnc(-c6nc(-c7ccccc7)nc(-c7ccc(-c8ccccc8)cc7)n6)cc4C54c5ccccc5Sc5ccccc54)n3)cc2)cc1] |
| 380 | 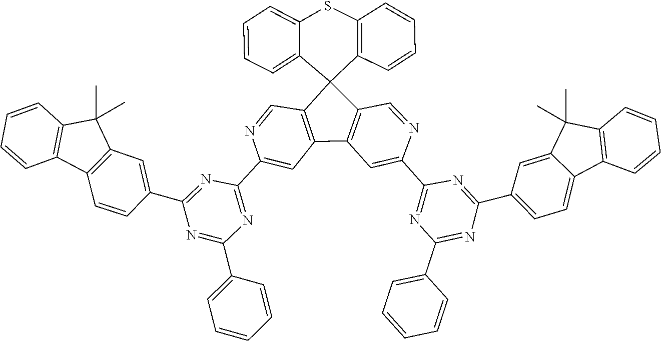 [SMILES: CC1(C)c2ccccc2-c2ccc(-c3nc(-c4ccccc4)nc(-c4cc5c(cn4)C4(c6ccccc6Sc6ccccc64)c4cnc(-c6nc(-c7ccccc7)nc(-c7ccc8c(c7)C(C)(C)c7ccccc7-8)n6)cc4-5)n3)cc21] |
| 381 | 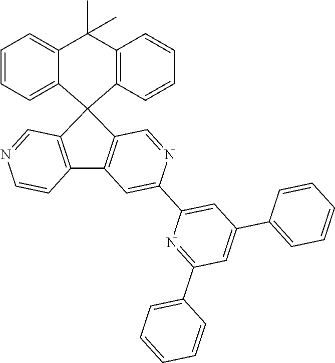 [SMILES: CC1(C)c2ccccc2C2(c3cnccc3-c3cc(-c4cc(-c5ccccc5)cc(-c5ccccc5)n4)ncc32)c2ccccc21] |
| 382 | 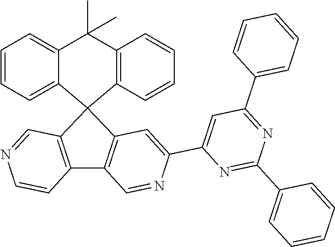 [SMILES: CC1(C)c2ccccc2C2(c3cnccc3-c3cnc(-c4cc(-c5ccccc5)nc(-c5ccccc5)n4)cc32)c2ccccc21] |
| 383 | 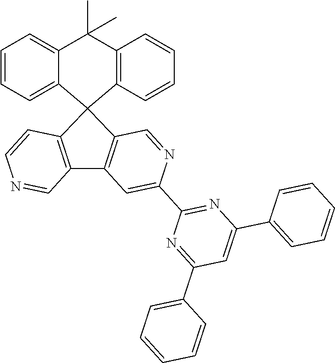 [SMILES: CC1(C)c2ccccc2C2(c3ccncc3-c3cc(-c4nc(-c5ccccc5)cc(-c5ccccc5)n4)ncc32)c2ccccc21] |
| 384 | 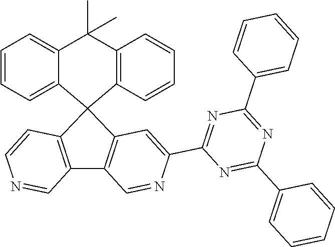 [SMILES: CC1(C)c2ccccc2C2(c3ccncc3-c3cnc(-c4nc(-c5ccccc5)nc(-c5ccccc5)n4)cc32)c2ccccc21] |
| 385 | 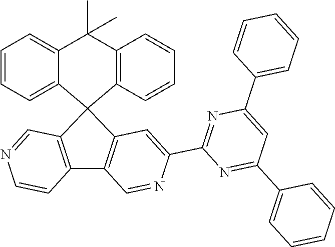 [SMILES: CC1(C)c2ccccc2C2(c3cnccc3-c3cnc(-c4nc(-c5ccccc5)cc(-c5ccccc5)n4)cc32)c2ccccc21] |
| 386 | 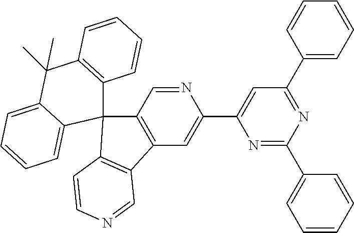 [SMILES: CC1(C)c2ccccc2C2(c3ccncc3-c3cc(-c4cc(-c5ccccc5)nc(-c5ccccc5)n4)ncc32)c2ccccc21] |
| 387 | 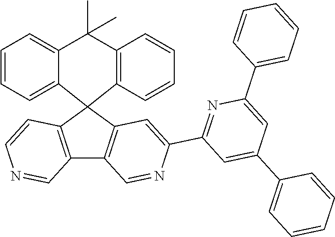 [SMILES: CC1(C)c2ccccc2C2(c3ccncc3-c3cnc(-c4cc(-c5ccccc5)cc(-c5ccccc5)n4)cc32)c2ccccc21] |
| 388 | 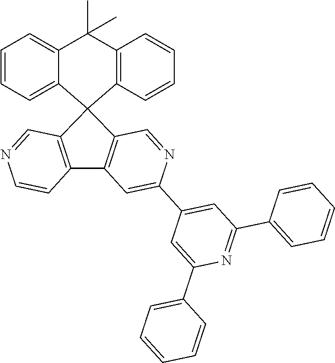 [SMILES: CC1(C)c2ccccc2C2(c3cnccc3-c3cc(-c4cc(-c5ccccc5)nc(-c5ccccc5)c4)ncc32)c2ccccc21] |
| 389 | 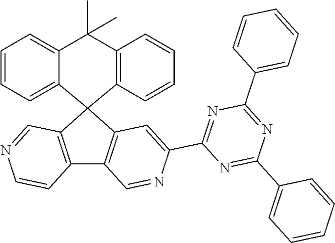 [SMILES: CC1(C)c2ccccc2C2(c3cnccc3-c3cnc(-c4nc(-c5ccccc5)nc(-c5ccccc5)n4)cc32)c2ccccc21] |
| 390 | 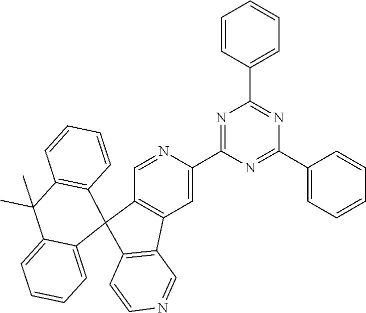 [SMILES: CC1(C)c2ccccc2C2(c3ccncc3-c3cc(-c4nc(-c5ccccc5)nc(-c5ccccc5)n4)ncc32)c2ccccc21] |
| 391 | 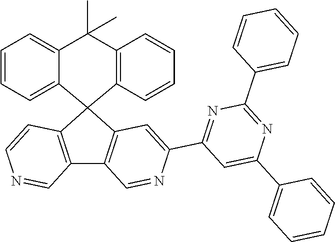 [SMILES: CC1(C)c2ccccc2C2(c3ccncc3-c3cnc(-c4cc(-c5ccccc5)nc(-c5ccccc5)n4)cc32)c2ccccc21] |
| 392 | 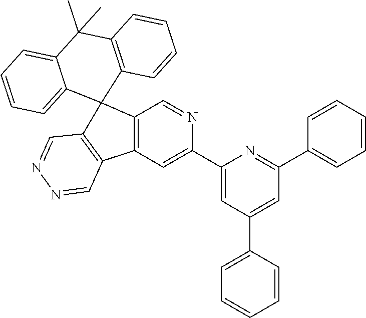 [SMILES: CC1(C)c2ccccc2C2(c3cnncc3-c3cc(-c4cc(-c5ccccc5)cc(-c5ccccc5)n4)ncc32)c2ccccc21] |
| 393 | 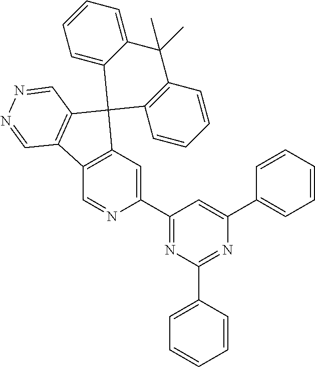 [SMILES: CC1(C)c2ccccc2C2(c3cnncc3-c3cnc(-c4cc(-c5ccccc5)nc(-c5ccccc5)n4)cc32)c2ccccc21] |
| 394 | 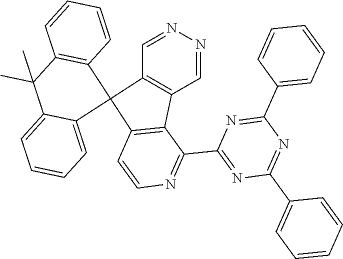 [SMILES: CC1(C)c2ccccc2C2(c3cnncc3-c3c2ccnc3-c2nc(-c3ccccc3)nc(-c3ccccc3)n2)c2ccccc21] |
| 395 | 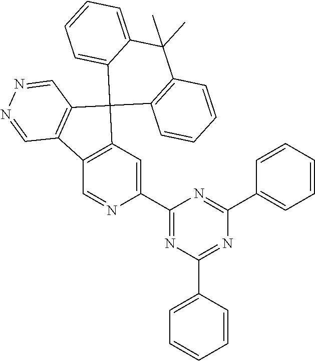 [SMILES: CC1(C)c2ccccc2C2(c3cnncc3-c3cnc(-c4nc(-c5ccccc5)nc(-c5ccccc5)n4)cc32)c2ccccc21] |
| 396 | 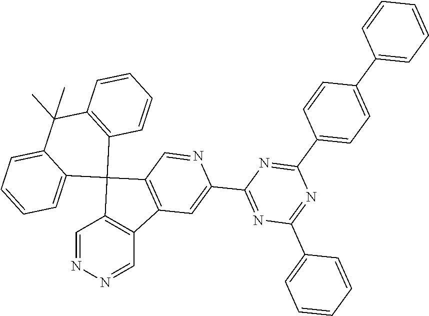 [SMILES: CC1(C)c2ccccc2C2(c3cnncc3-c3cc(-c4nc(-c5ccccc5)nc(-c5ccc(-c6ccccc6)cc5)n4)ncc32)c2ccccc21] |
| 397 | 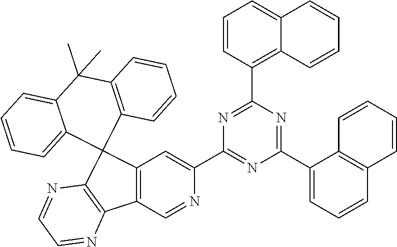 [SMILES: CC1(C)c2ccccc2C2(c3cc(-c4nc(-c5cccc6ccccc56)nc(-c5cccc6ccccc56)n4)ncc3-c3nccnc32)c2ccccc21] |
| 398 | 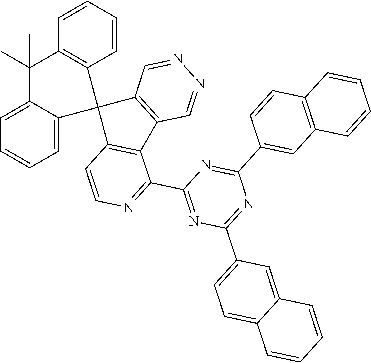 [SMILES: CC1(C)c2ccccc2C2(c3cnncc3-c3c2ccnc3-c2nc(-c3ccc4ccccc4c3)nc(-c3ccc4ccccc4c3)n2)c2ccccc21] |
| 399 | 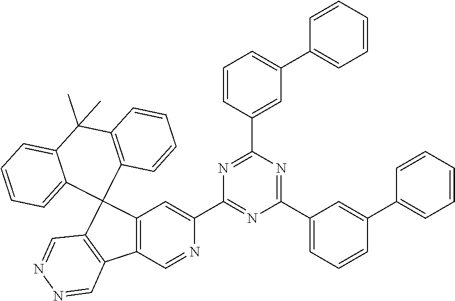 [SMILES: CC1(C)c2ccccc2C2(c3cnncc3-c3cnc(-c4nc(-c5cccc(-c6ccccc6)c5)nc(-c5cccc(-c6ccccc6)c5)n4)cc32)c2ccccc21] |
| 400 | 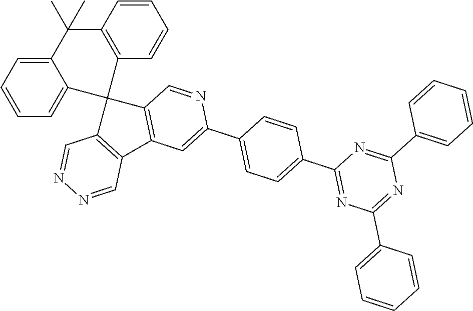 [SMILES: CC1(C)c2ccccc2C2(c3cnncc3-c3cc(-c4ccc(-c5nc(-c6ccccc6)nc(-c6ccccc6)n5)cc4)ncc32)c2ccccc21] |
| 401 | 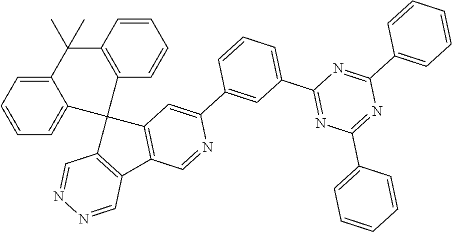 [SMILES: CC1(C)c2ccccc2C2(c3cnncc3-c3cnc(-c4cccc(-c5nc(-c6ccccc6)nc(-c6ccccc6)n5)c4)cc32)c2ccccc21] |
| 402 | 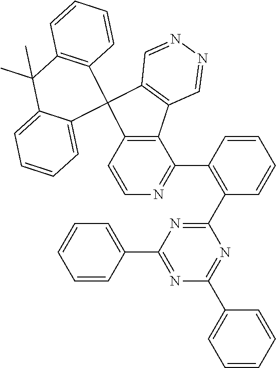 [SMILES: CC1(C)c2ccccc2C2(c3cnncc3-c3c2ccnc3-c2ccccc2-c2nc(-c3ccccc3)nc(-c3ccccc3)n2)c2ccccc21] |
| 403 | 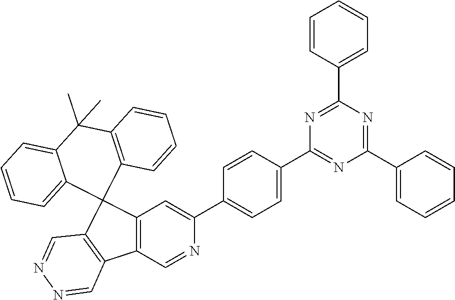 [SMILES: CC1(C)c2ccccc2C2(c3cnncc3-c3cnc(-c4ccc(-c5nc(-c6ccccc6)nc(-c6ccccc6)n5)cc4)cc32)c2ccccc21] |
| 404 | 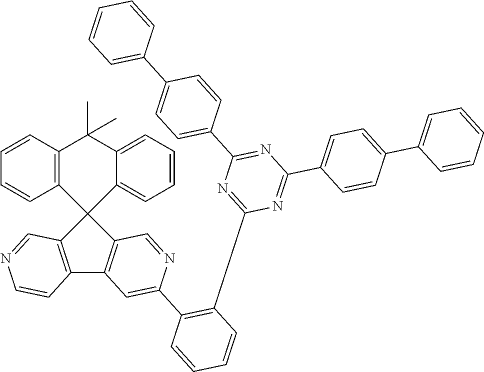 [SMILES: CC1(C)c2ccccc2C2(c3cnccc3-c3cc(-c4ccccc4-c4nc(-c5ccc(-c6ccccc6)cc5)nc(-c5ccc(-c6ccccc6)cc5)n4)ncc32)c2ccccc21] |
| 405 | 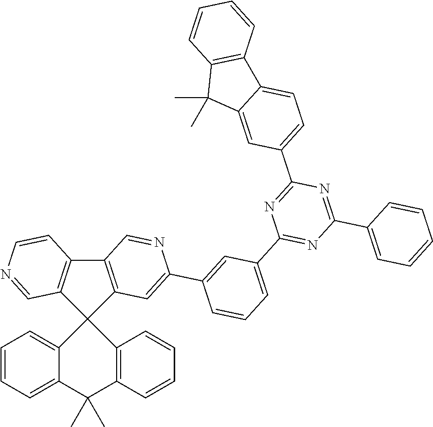 [SMILES: CC1(C)c2ccccc2-c2ccc(-c3nc(-c4ccccc4)nc(-c4cccc(-c5cc6c(cn5)-c5ccncc5C65c6ccccc6C(C)(C)c6ccccc65)c4)n3)cc21] |
| 406 | 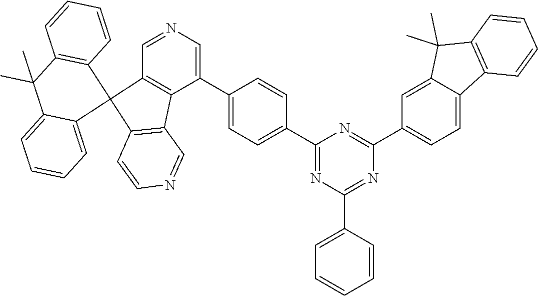 [SMILES: CC1(C)c2ccccc2-c2ccc(-c3nc(-c4ccccc4)nc(-c4ccc(-c5cncc6c5-c5cnccc5C65c6ccccc6C(C)(C)c6ccccc65)cc4)n3)cc21] |
| 407 | 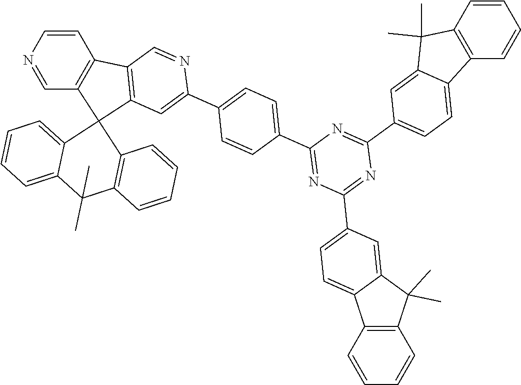 |
| 408 | 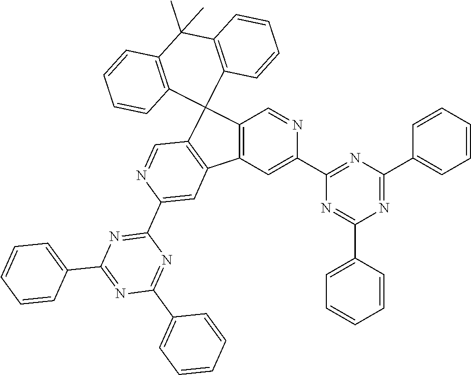 [SMILES: CC1(C)c2ccccc2C2(c3cnc(-c4nc(-c5ccccc5)nc(-c5ccccc5)n4)cc3-c3cc(-c4nc(-c5ccccc5)nc(-c5ccccc5)n4)ncc32)c2ccccc21] |
| 409 | 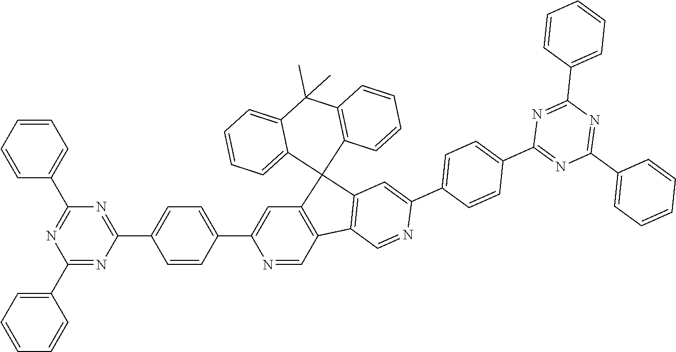 [SMILES: CC1(C)c2ccccc2C2(c3cc(-c4ccc(-c5nc(-c6ccccc6)nc(-c6ccccc6)n5)cc4)ncc3-c3cnc(-c4ccc(-c5nc(-c6ccccc6)nc(-c6ccccc6)n5)cc4)cc32)c2ccccc21] |
| 410 | 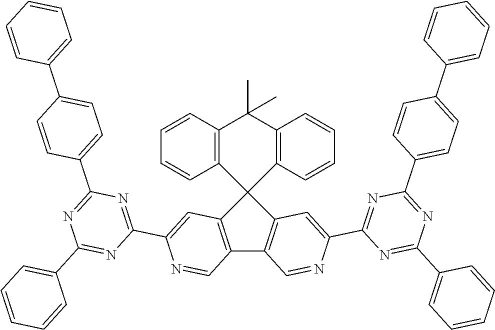 [SMILES: CC1(C)c2ccccc2C2(c3cc(-c4nc(-c5ccccc5)nc(-c5ccc(-c6ccccc6)cc5)n4)ncc3-c3cnc(-c4nc(-c5ccccc5)nc(-c5ccc(-c6ccccc6)cc5)n4)cc32)c2ccccc21] |
| 411 | 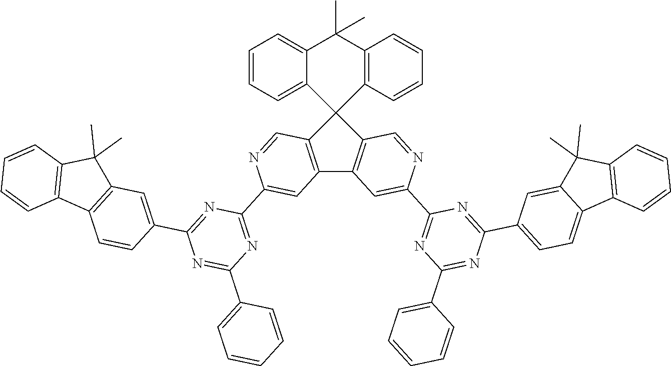 [SMILES: CC1(C)c2ccccc2-c2ccc(-c3nc(-c4ccccc4)nc(-c4cc5c(cn4)C4(c6cnc(-c7nc(-c8ccccc8)nc(-c8ccc9c(c8)C(C)(C)c8ccccc8-9)n7)cc6-5)c5ccccc5C(C)(C)c5ccccc54)n3)cc21] |
| 412 | 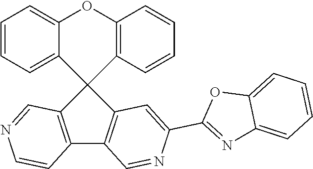 [SMILES: c1ccc2c(c1)Oc1ccccc1C21c2cnccc2-c2cnc(-c3nc4ccccc4o3)cc21] |
| 413 | 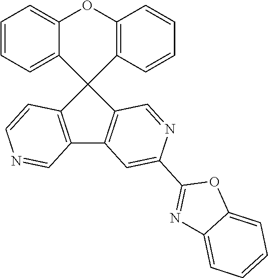 [SMILES: c1ccc2c(c1)Oc1ccccc1C21c2ccncc2-c2cc(-c3nc4ccccc4o3)ncc21] |
| 414 | 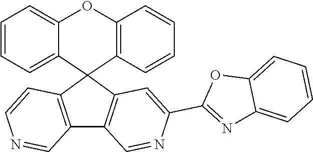 [SMILES: c1ccc2c(c1)Oc1ccccc1C21c2ccncc2-c2cnc(-c3nc4ccccc4o3)cc21] |
| 415 | 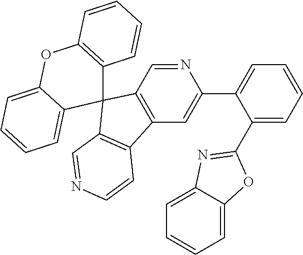 [SMILES: c1ccc2c(c1)Oc1ccccc1C21c2cnccc2-c2cc(-c3ccccc3-c3nc4ccccc4o3)ncc21] |
| 416 | 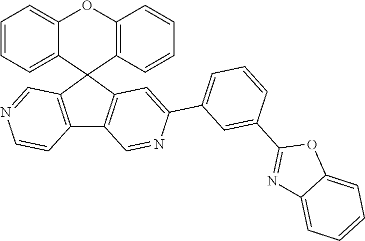 [SMILES: c1cc(-c2cc3c(cn2)-c2ccncc2C32c3ccccc3Oc3ccccc32)cc(-c2nc3ccccc3o2)c1] |
| 417 | 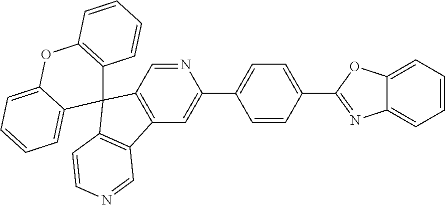 [SMILES: c1ccc2c(c1)Oc1ccccc1C21c2ccncc2-c2cc(-c3ccc(-c4nc5ccccc5o4)cc3)ncc21] |
| 418 | 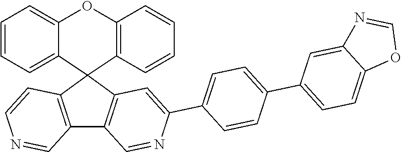 [SMILES: c1ccc2c(c1)Oc1ccccc1C21c2ccncc2-c2cnc(-c3ccc(-c4ccc5ocnc5c4)cc3)cc21] |
| 419 | 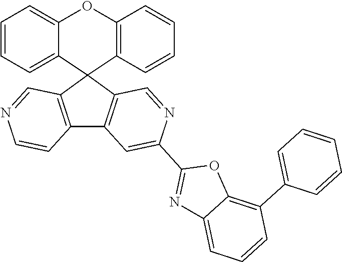 [SMILES: c1ccc(-c2cccc3nc(-c4cc5c(cn4)C4(c6ccccc6Oc6ccccc64)c4cnccc4-5)oc23)cc1] |
| 420 | 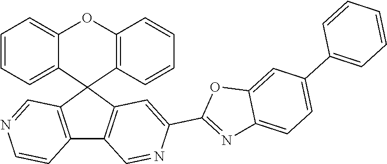 [SMILES: c1ccc(-c2ccc3nc(-c4cc5c(cn4)-c4ccncc4C54c5ccccc5Oc5ccccc54)oc3c2)cc1] |
| 421 | 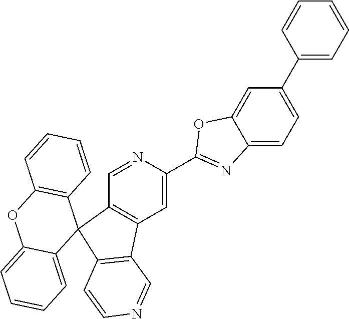 [SMILES: c1ccc(-c2ccc3nc(-c4cc5c(cn4)C4(c6ccccc6Oc6ccccc64)c4ccncc4-5)oc3c2)cc1] |
| 422 | 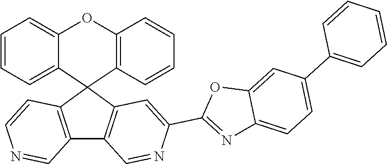 [SMILES: c1ccc(-c2ccc3nc(-c4cc5c(cn4)-c4cnccc4C54c5ccccc5Oc5ccccc54)oc3c2)cc1] |
| 423 | 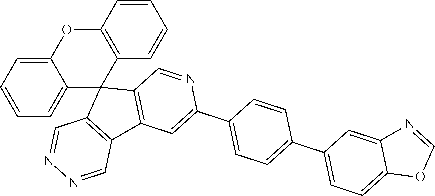 [SMILES: c1ccc2c(c1)Oc1ccccc1C21c2cnncc2-c2cc(-c3ccc(-c4ccc5ocnc5c4)cc3)ncc21] |
| 424 | 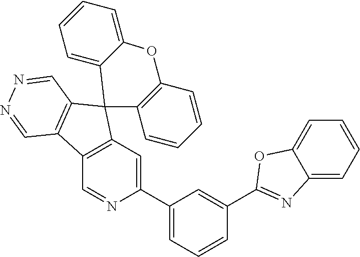 [SMILES: c1cc(-c2cc3c(cn2)-c2cnncc2C32c3ccccc3Oc3ccccc32)cc(-c2nc3ccccc3o2)c1] |
| 425 | 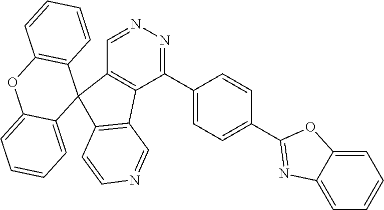 [SMILES: c1ccc2c(c1)Oc1ccccc1C21c2ccncc2-c2c1cnnc2-c1ccc(-c2nc3ccccc3o2)cc1] |
| 426 | 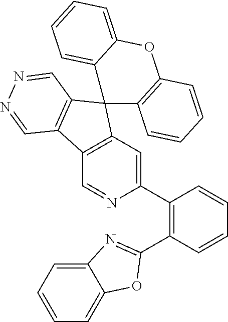 [SMILES: c1ccc2c(c1)Oc1ccccc1C21c2cnncc2-c2cnc(-c3ccccc3-c3nc4ccccc4o3)cc21] |
| 427 | 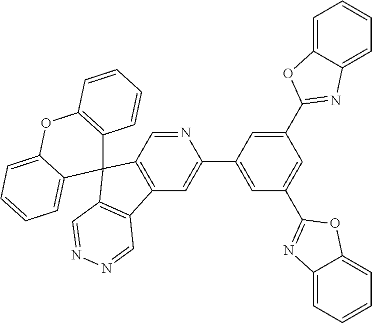 [SMILES: c1ccc2c(c1)Oc1ccccc1C21c2cnncc2-c2cc(-c3cc(-c4nc5ccccc5o4)cc(-c4nc5ccccc5o4)c3)ncc21] |
| 428 | 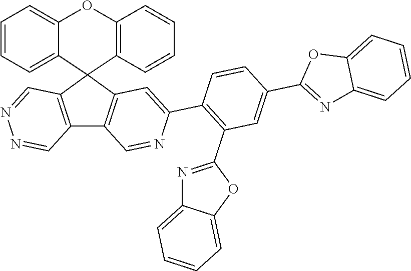 [SMILES: c1ccc2c(c1)Oc1ccccc1C21c2cnncc2-c2cnc(-c3ccc(-c4nc5ccccc5o4)cc3-c3nc4ccccc4o3)cc21] |
| 429 | 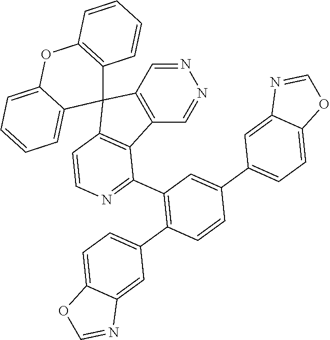 [SMILES: c1ccc2c(c1)Oc1ccccc1C21c2cnncc2-c2c1ccnc2-c1cc(-c2ccc3ocnc3c2)ccc1-c1ccc2ocnc2c1] |
| 430 | 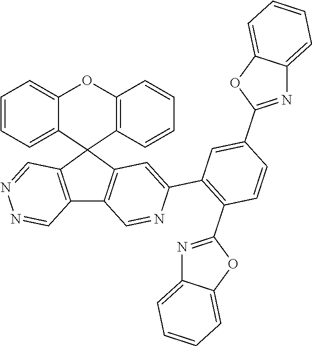 [SMILES: c1ccc2c(c1)Oc1ccccc1C21c2cnncc2-c2cnc(-c3cc(-c4nc5ccccc5o4)ccc3-c3nc4ccccc4o3)cc21] |
| 431 | 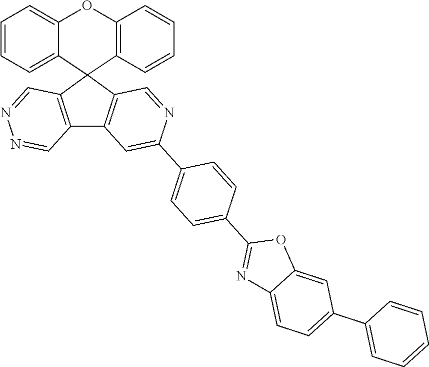 [SMILES: c1ccc(-c2ccc3nc(-c4ccc(-c5cc6c(cn5)C5(c7ccccc7Oc7ccccc75)c5cnncc5-6)cc4)oc3c2)cc1] |
| 432 | 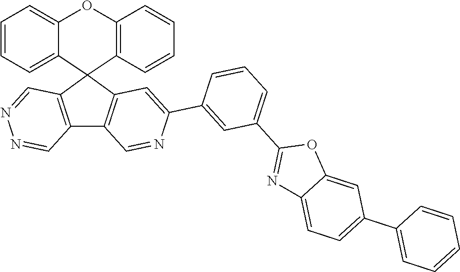 [SMILES: c1ccc(-c2ccc3nc(-c4cccc(-c5cc6c(cn5)-c5cnncc5C65c6ccccc6Oc6ccccc65)c4)oc3c2)cc1] |
| 433 | 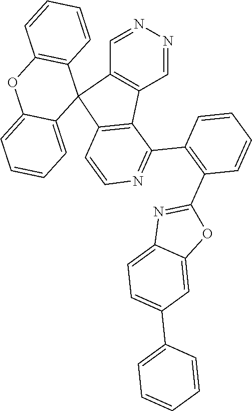 [SMILES: c1ccc(-c2ccc3nc(-c4ccccc4-c4nccc5c4-c4cnncc4C54c5ccccc5Oc5ccccc54)oc3c2)cc1] |
| 434 | 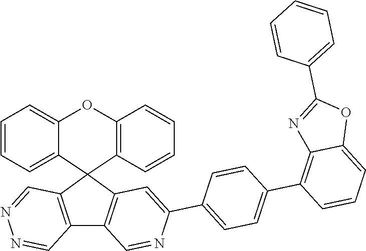 [SMILES: c1ccc(-c2nc3c(-c4ccc(-c5cc6c(cn5)-c5cnncc5C65c6ccccc6Oc6ccccc65)cc4)cccc3o2)cc1] |
| 435 | 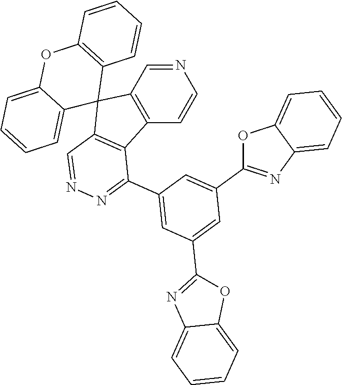 [SMILES: c1ccc2c(c1)Oc1ccccc1C21c2cnccc2-c2c1cnnc2-c1cc(-c2nc3ccccc3o2)cc(-c2nc3ccccc3o2)c1] |
| 436 | 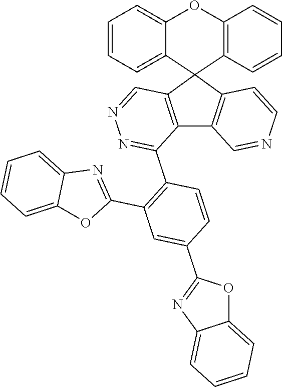 [SMILES: c1ccc2c(c1)Oc1ccccc1C21c2ccncc2-c2c1cnnc2-c1ccc(-c2nc3ccccc3o2)cc1-c1nc2ccccc2o1] |
| 437 | 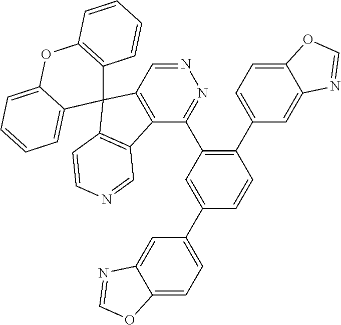 [SMILES: c1ccc2c(c1)Oc1ccccc1C21c2ccncc2-c2c1cnnc2-c1cc(-c2ccc3ocnc3c2)ccc1-c1ccc2ocnc2c1] |
| 438 | 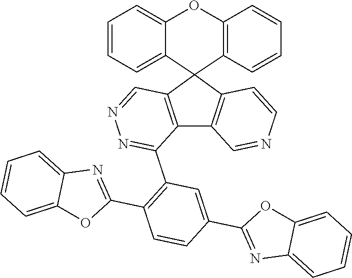 [SMILES: c1ccc2c(c1)Oc1ccccc1C21c2ccncc2-c2c1cnnc2-c1cc(-c2nc3ccccc3o2)ccc1-c1nc2ccccc2o1] |
| 439 | 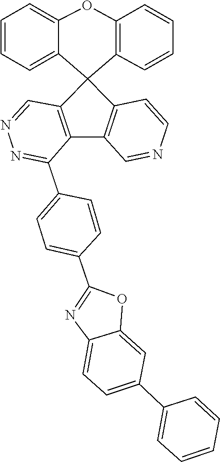 [SMILES: c1ccc(-c2ccc3nc(-c4ccc(-c5nncc6c5-c5cnccc5C65c6ccccc6Oc6ccccc65)cc4)oc3c2)cc1] |
| 440 | 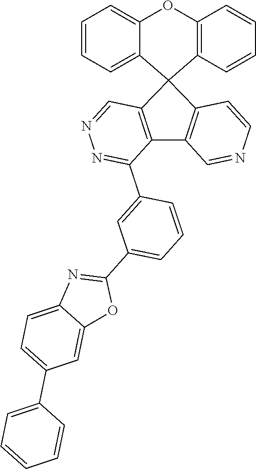 [SMILES: c1ccc(-c2ccc3nc(-c4cccc(-c5nncc6c5-c5cnccc5C65c6ccccc6Oc6ccccc65)c4)oc3c2)cc1] |
| 441 | 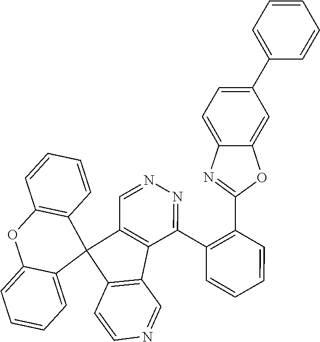 [SMILES: c1ccc(-c2ccc3nc(-c4ccccc4-c4nncc5c4-c4cnccc4C54c5ccccc5Oc5ccccc54)oc3c2)cc1] |
| 442 | 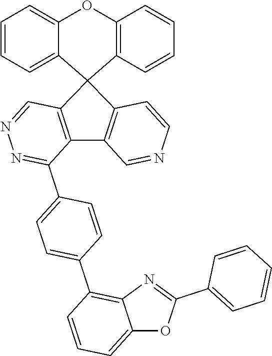 [SMILES: c1ccc(-c2nc3c(-c4ccc(-c5nncc6c5-c5cnccc5C65c6ccccc6Oc6ccccc65)cc4)cccc3o2)cc1] |
| 443 | 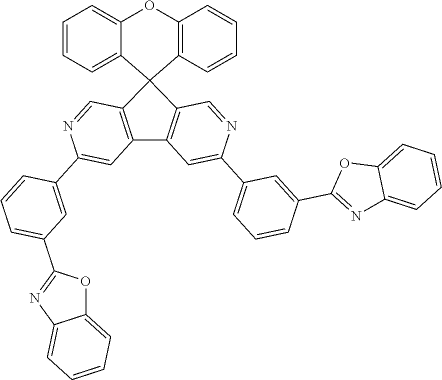 [SMILES: c1cc(-c2cc3c(cn2)C2(c4ccccc4Oc4ccccc42)c2cnc(-c4cccc(-c5nc6ccccc6o5)c4)cc2-3)cc(-c2nc3ccccc3o2)c1] |
| 444 | 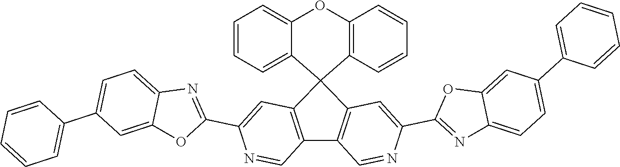 [SMILES: c1ccc(-c2ccc3nc(-c4cc5c(cn4)-c4cnc(-c6nc7ccc(-c8ccccc8)cc7o6)cc4C54c5ccccc5Oc5ccccc54)oc3c2)cc1] |
| 445 | 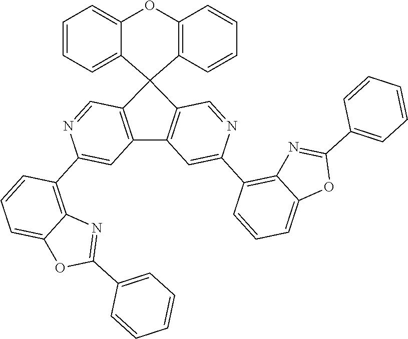 [SMILES: c1ccc(-c2nc3c(-c4cc5c(cn4)C4(c6ccccc6Oc6ccccc64)c4cnc(-c6cccc7oc(-c8ccccc8)nc67)cc4-5)cccc3o2)cc1] |
| 446 | 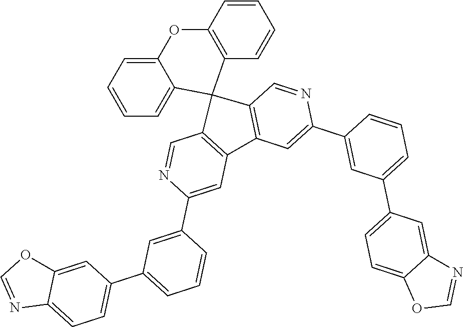 [SMILES: c1cc(-c2ccc3ocnc3c2)cc(-c2cc3c(cn2)C2(c4ccccc4Oc4ccccc42)c2cnc(-c4cccc(-c5ccc6ncoc6c5)c4)cc2-3)c1] |
| 447 | 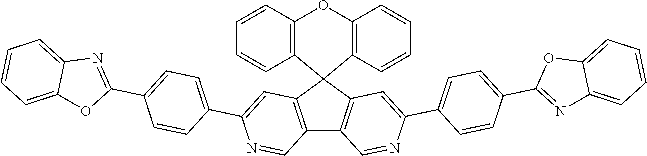 [SMILES: c1ccc2c(c1)Oc1ccccc1C21c2cc(-c3ccc(-c4nc5ccccc5o4)cc3)ncc2-c2cnc(-c3ccc(-c4nc5ccccc5o4)cc3)cc21] |
| 448 | 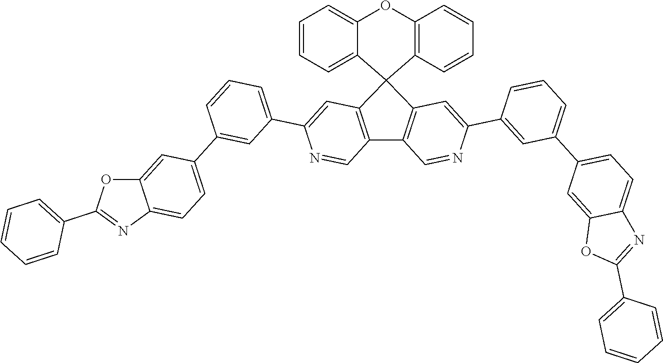 [SMILES: c1ccc(-c2nc3ccc(-c4cccc(-c5cc6c(cn5)-c5cnc(-c7cccc(-c8ccc9nc(-c%10ccccc%10)oc9c8)c7)cc5C65c6ccccc6Oc6ccccc65)c4)cc3o2)cc1] |
| 449 | 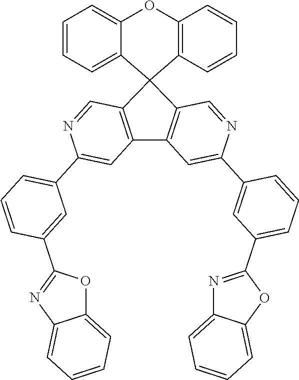 [SMILES: c1cc(-c2cc3c(cn2)C2(c4ccccc4Oc4ccccc42)c2cnc(-c4cccc(-c5nc6ccccc6o5)c4)cc2-3)cc(-c2nc3ccccc3o2)c1] |
| 450 | 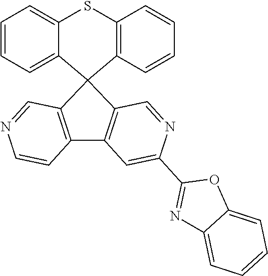 [SMILES: c1ccc2c(c1)Sc1ccccc1C21c2cnccc2-c2cc(-c3nc4ccccc4o3)ncc21] |
| 451 | 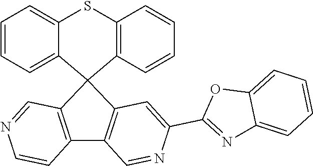 [SMILES: c1ccc2c(c1)Sc1ccccc1C21c2cnccc2-c2cnc(-c3nc4ccccc4o3)cc21] |
| 452 | 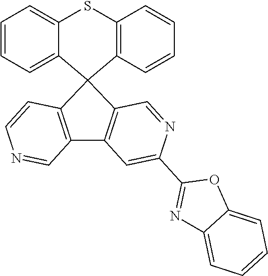 [SMILES: c1ccc2c(c1)Sc1ccccc1C21c2ccncc2-c2cc(-c3nc4ccccc4o3)ncc21] |
| 453 | 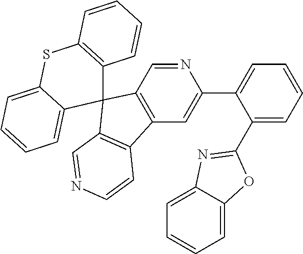 [SMILES: c1ccc2c(c1)Sc1ccccc1C21c2cnccc2-c2cc(-c3ccccc3-c3nc4ccccc4o3)ncc21] |
| 454 | 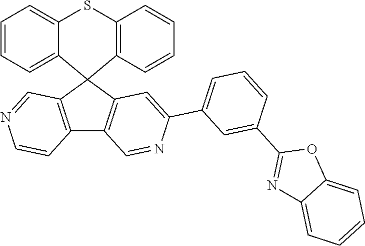 [SMILES: c1cc(-c2cc3c(cn2)-c2ccncc2C32c3ccccc3Sc3ccccc32)cc(-c2nc3ccccc3o2)c1] |
| 455 | 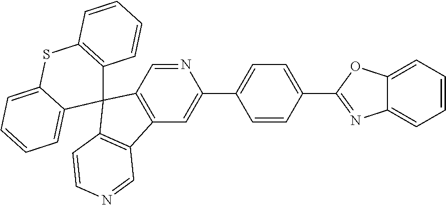 [SMILES: c1ccc2c(c1)Sc1ccccc1C21c2ccncc2-c2cc(-c3ccc(-c4nc5ccccc5o4)cc3)ncc21] |
| 456 | 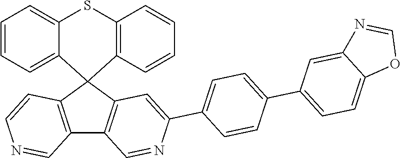 [SMILES: c1ccc2c(c1)Sc1ccccc1C21c2ccncc2-c2cnc(-c3ccc(-c4ccc5ocnc5c4)cc3)cc21] |
| 457 | 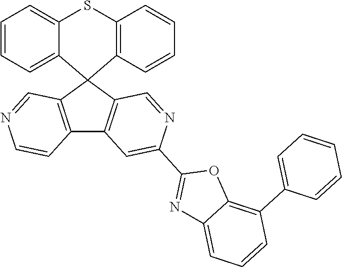 [SMILES: c1ccc(-c2cccc3nc(-c4cc5c(cn4)C4(c6ccccc6Sc6ccccc64)c4cnccc4-5)oc23)cc1] |
| 458 | 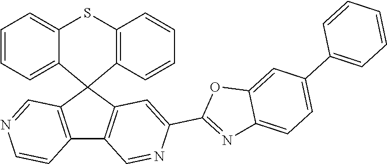 [SMILES: c1ccc(-c2ccc3nc(-c4cc5c(cn4)-c4ccncc4C54c5ccccc5Sc5ccccc54)oc3c2)cc1] |
| 459 | 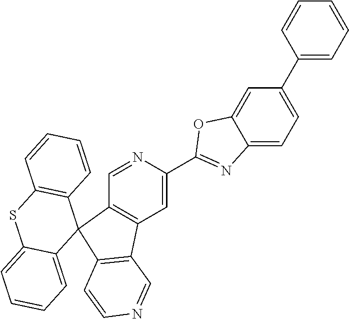 [SMILES: c1ccc(-c2ccc3nc(-c4cc5c(cn4)C4(c6ccccc6Sc6ccccc64)c4ccncc4-5)oc3c2)cc1] |
| 460 | 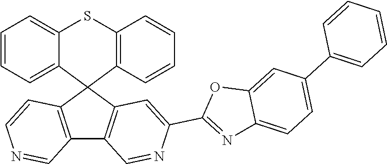 [SMILES: c1ccc(-c2ccc3nc(-c4cc5c(cn4)-c4cnccc4C54c5ccccc5Sc5ccccc54)oc3c2)cc1] |
| 461 | 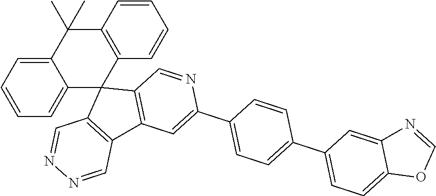 [SMILES: CC1(C)c2ccccc2C2(c3cnncc3-c3cc(-c4ccc(-c5ccc6ocnc6c5)cc4)ncc32)c2ccccc21] |
| 462 | 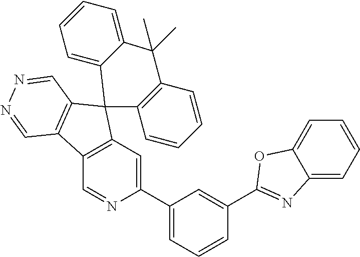 [SMILES: CC1(C)c2ccccc2C2(c3cnncc3-c3cnc(-c4cccc(-c5nc6ccccc6o5)c4)cc32)c2ccccc21] |
| 463 | 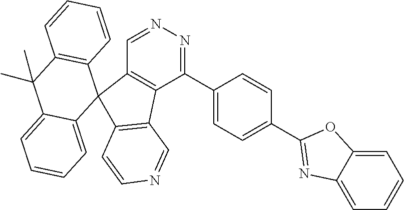 [SMILES: CC1(C)c2ccccc2C2(c3ccncc3-c3c2cnnc3-c2ccc(-c3nc4ccccc4o3)cc2)c2ccccc21] |
| 464 | 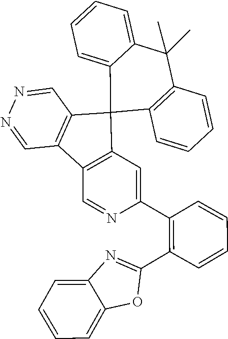 [SMILES: CC1(C)c2ccccc2C2(c3cnncc3-c3cnc(-c4ccccc4-c4nc5ccccc5o4)cc32)c2ccccc21] |
| 465 | 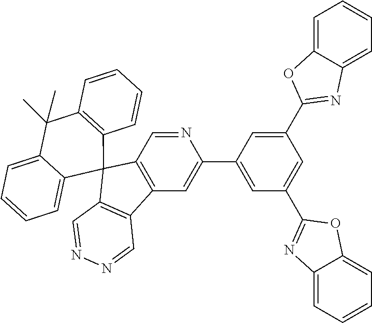 [SMILES: CC1(C)c2ccccc2C2(c3cnncc3-c3cc(-c4cc(-c5nc6ccccc6o5)cc(-c5nc6ccccc6o5)c4)ncc32)c2ccccc21] |
| 466 | 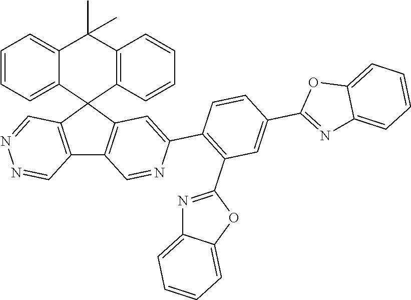 [SMILES: CC1(C)c2ccccc2C2(c3cnncc3-c3cnc(-c4ccc(-c5nc6ccccc6o5)cc4-c4nc5ccccc5o4)cc32)c2ccccc21] |
| 467 | 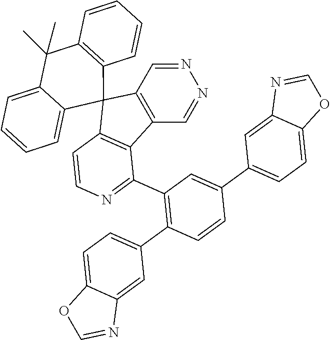 [SMILES: CC1(C)c2ccccc2C2(c3cnncc3-c3c2ccnc3-c2cc(-c3ccc4ocnc4c3)ccc2-c2ccc3ocnc3c2)c2ccccc21] |
| 468 | 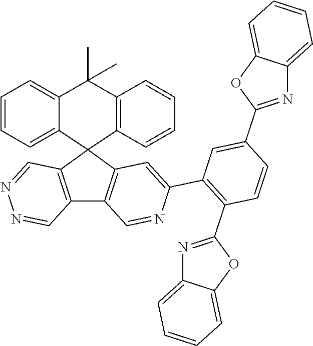 [SMILES: CC1(C)c2ccccc2C2(c3cnncc3-c3cnc(-c4cc(-c5nc6ccccc6o5)ccc4-c4nc5ccccc5o4)cc32)c2ccccc21] |
| 469 | 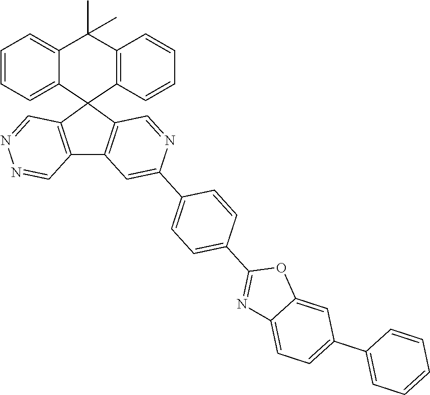 [SMILES: CC1(C)c2ccccc2C2(c3cnncc3-c3cc(-c4ccc(-c5nc6ccc(-c7ccccc7)cc6o5)cc4)ncc32)c2ccccc21] |
| 470 | 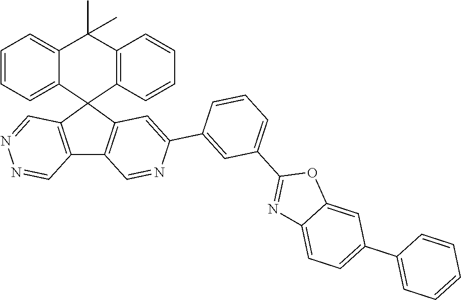 [SMILES: CC1(C)c2ccccc2C2(c3cnncc3-c3cnc(-c4cccc(-c5nc6ccc(-c7ccccc7)cc6o5)c4)cc32)c2ccccc21] |
| 471 | 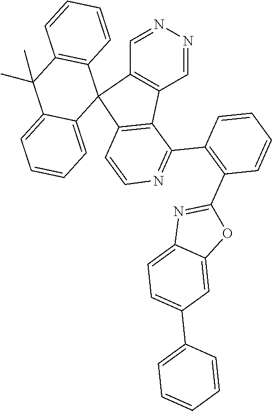 [SMILES: CC1(C)c2ccccc2C2(c3cnncc3-c3c2ccnc3-c2ccccc2-c2nc3ccc(-c4ccccc4)cc3o2)c2ccccc21] |
| 472 | 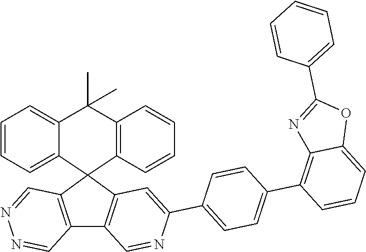 [SMILES: CC1(C)c2ccccc2C2(c3cnncc3-c3cnc(-c4ccc(-c5cccc6oc(-c7ccccc7)nc56)cc4)cc32)c2ccccc21] |
| 473 | 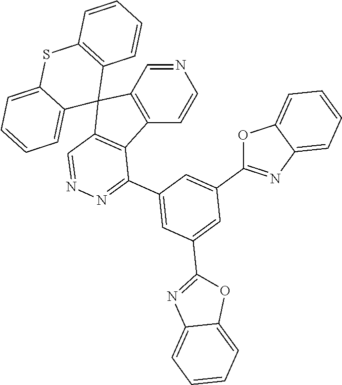 [SMILES: c1ccc2c(c1)Sc1ccccc1C21c2cnccc2-c2c1cnnc2-c1cc(-c2nc3ccccc3o2)cc(-c2nc3ccccc3o2)c1] |
| 474 | 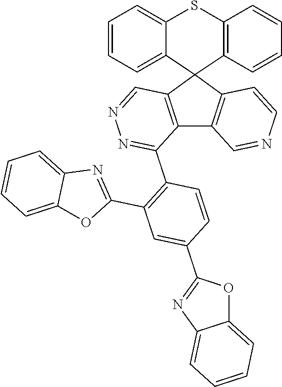 [SMILES: c1ccc2c(c1)Sc1ccccc1C21c2ccncc2-c2c1cnnc2-c1ccc(-c2nc3ccccc3o2)cc1-c1nc2ccccc2o1] |
| 475 | 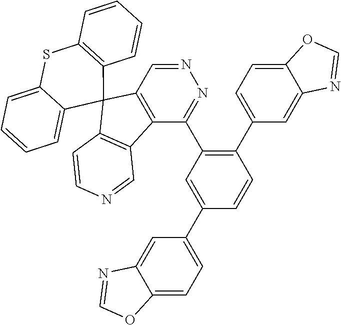 [SMILES: c1ccc2c(c1)Sc1ccccc1C21c2ccncc2-c2c1cnnc2-c1cc(-c2ccc3ocnc3c2)ccc1-c1ccc2ocnc2c1] |
| 476 | 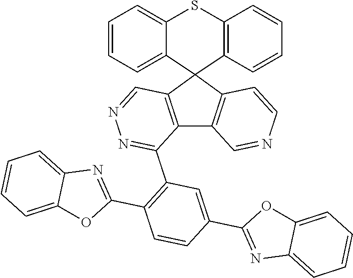 [SMILES: c1ccc2c(c1)Sc1ccccc1C21c2ccncc2-c2c1cnnc2-c1cc(-c2nc3ccccc3o2)ccc1-c1nc2ccccc2o1] |
| 477 | 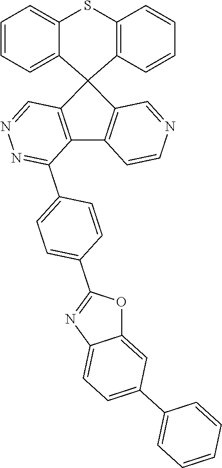 [SMILES: c1ccc(-c2ccc3nc(-c4ccc(-c5nncc6c5-c5ccncc5C65c6ccccc6Sc6ccccc65)cc4)oc3c2)cc1] |
| 478 | 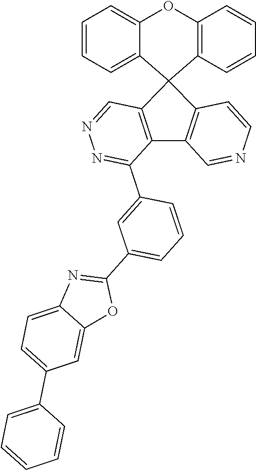 [SMILES: c1ccc(-c2ccc3nc(-c4cccc(-c5nncc6c5-c5cnccc5C65c6ccccc6Oc6ccccc65)c4)oc3c2)cc1] |
| 479 | 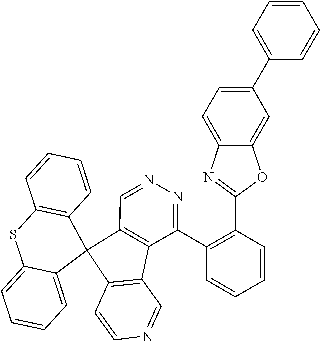 [SMILES: c1ccc(-c2ccc3nc(-c4ccccc4-c4nncc5c4-c4cnccc4C54c5ccccc5Sc5ccccc54)oc3c2)cc1] |
| 480 | 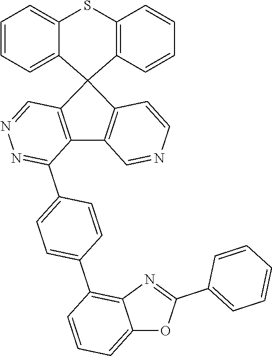 [SMILES: c1ccc(-c2nc3c(-c4ccc(-c5nncc6c5-c5cnccc5C65c6ccccc6Sc6ccccc65)cc4)cccc3o2)cc1] |
| 481 | 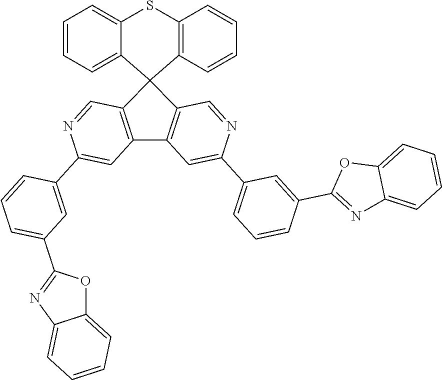 [SMILES: c1cc(-c2cc3c(cn2)C2(c4ccccc4Sc4ccccc42)c2cnc(-c4cccc(-c5nc6ccccc6o5)c4)cc2-3)cc(-c2nc3ccccc3o2)c1] |
| 482 | 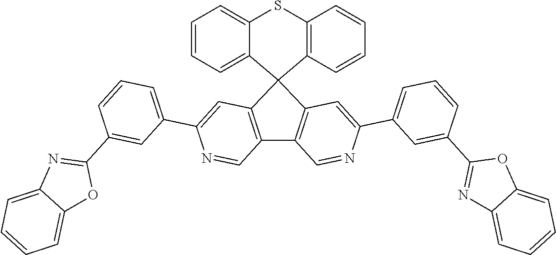 [SMILES: c1cc(-c2cc3c(cn2)-c2cnc(-c4cccc(-c5nc6ccccc6o5)c4)cc2C32c3ccccc3Sc3ccccc32)cc(-c2nc3ccccc3o2)c1] |
| 483 | 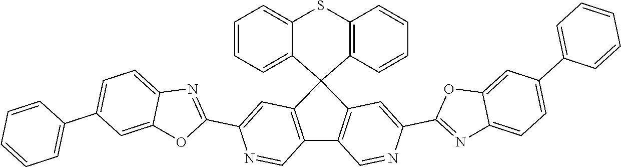 [SMILES: c1ccc(-c2ccc3nc(-c4cc5c(cn4)-c4cnc(-c6nc7ccc(-c8ccccc8)cc7o6)cc4C54c5ccccc5Sc5ccccc54)oc3c2)cc1] |
| 484 | 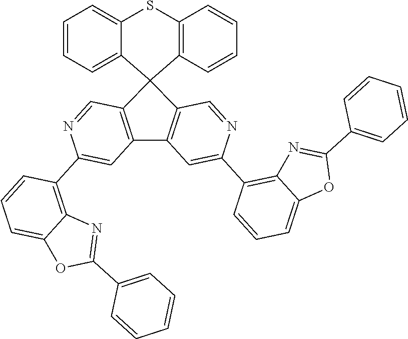 [SMILES: c1ccc(-c2nc3c(-c4cc5c(cn4)C4(c6ccccc6Sc6ccccc64)c4cnc(-c6cccc7oc(-c8ccccc8)nc67)cc4-5)cccc3o2)cc1] |
| 485 | 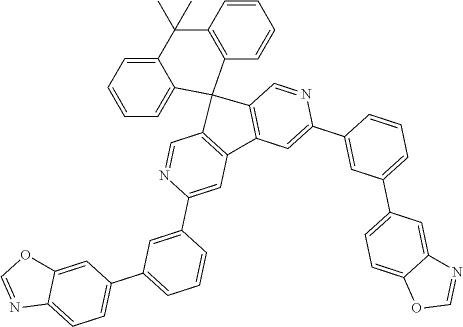 [SMILES: CC1(C)c2ccccc2C2(c3cnc(-c4cccc(-c5ccc6ocnc6c5)c4)cc3-c3cc(-c4cccc(-c5ccc6ncoc6c5)c4)ncc32)c2ccccc21] |
| 486 | 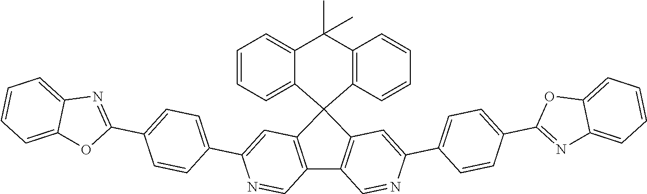 [SMILES: CC1(C)c2ccccc2C2(c3cc(-c4ccc(-c5nc6ccccc6o5)cc4)ncc3-c3cnc(-c4ccc(-c5nc6ccccc6o5)cc4)cc32)c2ccccc21] |
| 487 | 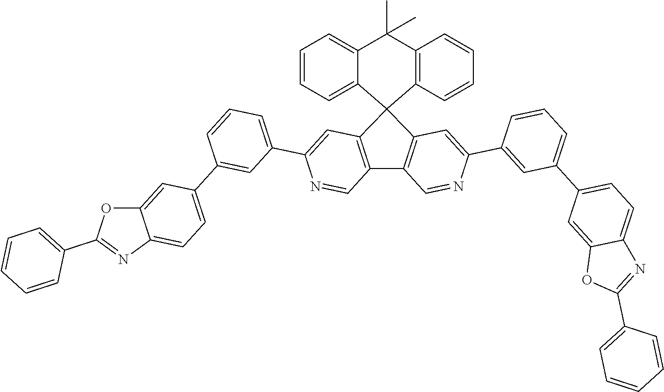 [SMILES: CC1(C)c2ccccc2C2(c3cc(-c4cccc(-c5ccc6nc(-c7ccccc7)oc6c5)c4)ncc3-c3cnc(-c4cccc(-c5ccc6nc(-c7ccccc7)oc6c5)c4)cc32)c2ccccc21] |
| 488 | 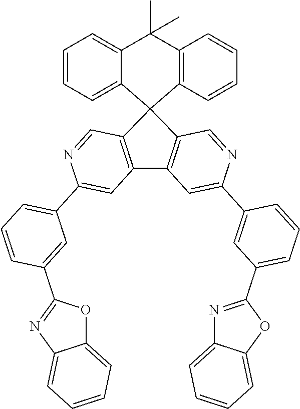 [SMILES: CC1(C)c2ccccc2C2(c3cnc(-c4cccc(-c5nc6ccccc6o5)c4)cc3-c3cc(-c4cccc(-c5nc6ccccc6o5)c4)ncc32)c2ccccc21] |

20. An electron transport material, which comprises the compound, or a stereoisomer or tautomer thereof according to claim 1.

21. An organic electroluminescent device, which comprises an electron transport layer, and the material of the electron transport layer comprises the compound, or a stereoisomer or tautomer thereof according to claim 1;
preferably, the organic electroluminescent device comprises a first electrode, a hole injection layer, a hole transport layer, an electron blocking layer, a light emitting layer, a hole blocking layer, an electron transport layer, an electron injection layer and a second electrode that are stacked in sequence, and the material of the electron transport layer comprises the compound, or a stereoisomer or tautomer thereof according to claim 1.

22. An electronic device, which comprises the organic electroluminescent device according to claim 21.

23. The compound, or a stereoisomer or tautomer thereof according to claim 1, wherein Ring A1 and Ring A2 are each independently selected from the group consisting of benzene ring and naphthalene ring;
preferably, Ring A1 is selected from the group consisting of 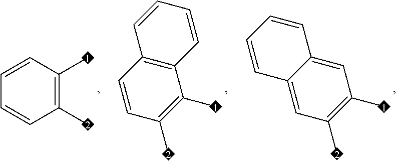
wherein Carbon atom No. 1 is fused with the carbon atom adjacent to Y on the middle 6-membered ring, and Carbon atom No. 2 is fused with the carbon atom far away from Y on the middle 6-membered ring;
more preferably, Ring A1 is 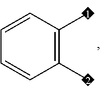
wherein Carbon atom No. 1 is fused with the carbon atom adjacent to Y on the middle 6-membered ring, and Carbon atom No. 2 is fused with the carbon atom far away from Y on the middle 6-membered ring;
preferably, Ring A2 is selected from the group consisting of 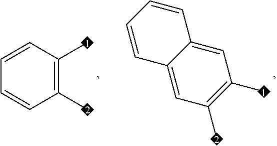
wherein Carbon atom No. 1 is fused with the carbon atom adjacent to Y on the middle 6-membered ring, and Carbon atom No. 2 is fused with the carbon atom far away from Y on the middle 6-membered ring;
more preferably, Ring A2 is 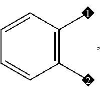
wherein Carbon atom No. 1 is fused with the carbon atom adjacent to Y on the middle 6-membered ring, and Carbon atom No. 2 is fused with the carbon atom far away from Y on the middle 6-membered ring;
or, wherein 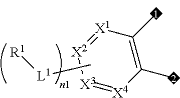
is selected from the group consisting of: 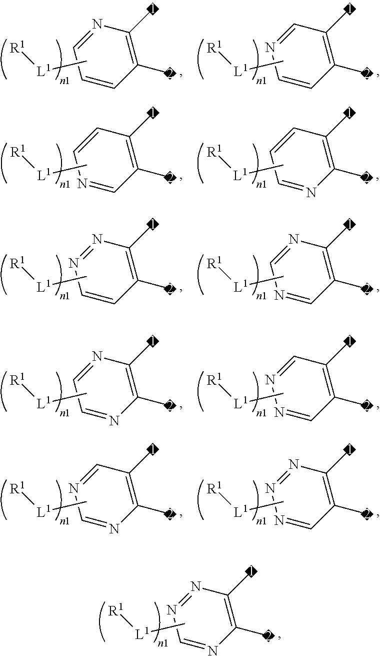
wherein, Carbon atom No. 1 is bonded to the central spiro carbon atom, and Carbon atom No. 2 is bonded to a carbon atom of the aromatic ring on the right;
preferably, 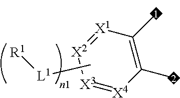
is selected from the group consisting of: 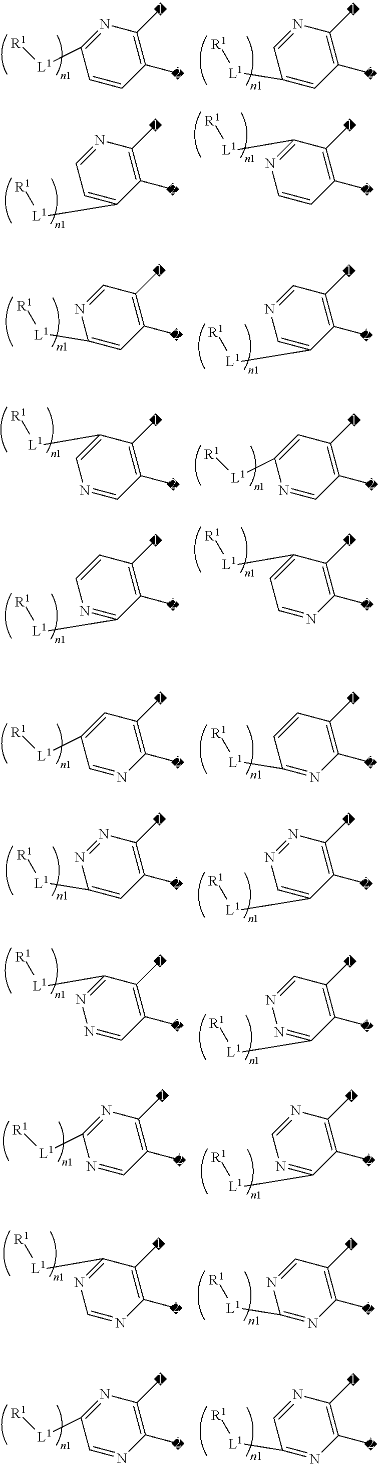 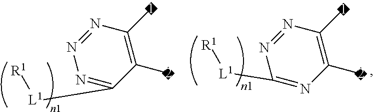
wherein, Carbon atom No. 1 is bonded to the central spiro carbon atom, and Carbon atom No. 2 is bonded to a carbon atom of the aromatic ring on the right;
or, wherein 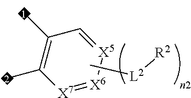
is selected from the group consisting of: 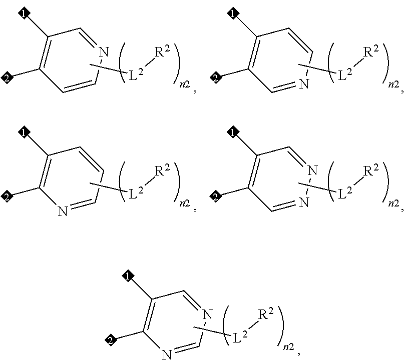
wherein, Carbon atom No. 1 is bonded to the central spiro carbon atom, and Carbon atom No. 2 is bonded to a carbon atom of the aromatic ring on the left;
preferably, 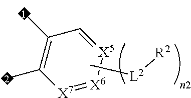
is selected from the group consisting of: 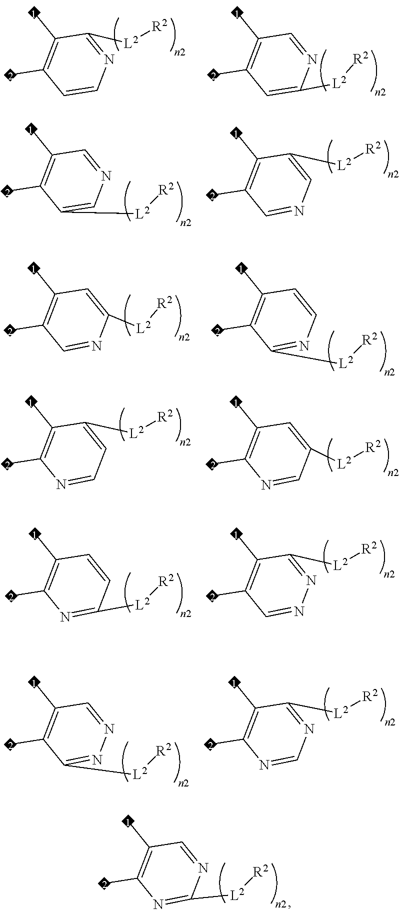
wherein, Carbon atom No. 1 is bonded to the central spiro carbon atom, and Carbon atom No. 2 is bonded to a carbon atom of the aromatic ring on the left.
